(19) United States
(12) Patent Application Publication
(10) Pub. No.: US 2007/0135383 A1
(43) Pub. Date: Jun. 14, 2007

(54) Phospholipase inhibitors, including multi-valent phospholipase inhibitors, and use thereof, including as lumen-localized phospholipase inhibitors

(76) Inventors: Han-Ting Chang, Livermore, CA (US); Dominque Charmot, Campbell, CA (US); Tomasz Glinka, Cupertino, CA (US); Michael James Cope, Berkeley, CA (US); Elizabeth Goka, San Jose, CA (US); Jun Shao, Fremont, CA (US); Damien Cartigny, Rouen (FR); Shiah-yun Chen, Mountain View, CA (US); Jerry M. Buysse, Lost Altos, CA (US)

Correspondence Address: ILYPSA-McANDREWS;C/O MCANDREWS, HELD & MADISON STREET, 500 WEST MADISON STREET, SUITE 3400, CHICAGO, IL, 60661, US

(21) Appl. No.: 11/593,176

(22) Filed: Nov. 3, 2006

Related U.S. Application Data
(60) Provisional application No. 60/734,037, filed on Nov. 3, 2005.

(51) Int. Cl. A61K 31/685; A61K 31/66; A61K 31/503; A61K 31/4745; A61K 31/405; A61K 31/365

(52) U.S. Cl. 514/78; 514/129; 514/472; 514/300; 514/415; 514/248; 514/414; 514/419

(57) ABSTRACT

The present invention provides methods and compositions for the treatment of phospholipase-related conditions. In particular, the invention provides a method of treating insulin-related, weight-related conditions and/or cholesterol-related conditions in an animal subject. The method generally involves the administration of a non-absorbed and/or effluxed phospholipase A2 inhibitor that is localized in a gastrointestinal lumen.

### RELATED APPLICATION

This application is related to co-owned, co-pending U.S. patent application Ser. No. 10/838,879 entitled “Phospholipase Inhibitors Localized in the Gastrointestinal Lumen” filed May 3, 2004 by Hui et al., and also to PCT Patent Application No. US 2005/015418 entitled “Phospholipase Inhibitors Localized in the Gastrointestinal Lumen” filed May 3, 2005 by Ilypsa, Inc., each of which is incorporated by reference herein in its entirety. This application claim benefit of a provisional application of the same title, 60/734,037, filed Nov. 3, 2005.

### BACKGROUND OF THE INVENTION

Phospholipases are a group of enzymes that play important roles in a number of biochemical processes, including regulation of membrane fluidity and stability, digestion and metabolism of phospholipids, and production of intracellular messengers involved in inflammatory pathways, hemodynamic regulation and other cellular processes. Phospholipases are themselves regulated by a number of mechanisms, including selective phosphorylation, pH, and intracellular calcium levels. Phospholipase activities can be modulated to regulate their related biochemical processes, and a number of phospholipase inhibitors have been developed.

Certain phospholipase activities occur in the gastrointestinal lumen, for example, phospholipase A₂ acts in the digestion of dietary phospholipids in the gastrointestinal lumen, and phospholipase B is active in the apical mucosa of the distal intestine. The activities of these enzymes affect a number of phospholipase-related conditions, including diabetes, weight gain and cholesterol-related conditions.

Diabetes affects 18.2 million people in the Unites States, representing over 6% of the population. Diabetes is characterized by the inability to produce or properly use insulin. Diabetes type 2 (also called non-insulin-dependent diabetes or NIDDM) accounts for 80-90% of the diagnosed cases of diabetes and is caused by insulin resistance. Insulin resistance in diabetes type 2 prevents maintenance of blood glucose within desirable ranges, despite normal to elevated plasma levels of insulin.

Obesity is a major contributor to diabetes type 2, as well as other illnesses including coronary heart disease, osteoarthritis, respiratory problems, and certain cancers. Despite attempts to control weight gain, obesity remains a serious health concern in the United States and other industrialized countries. Indeed, over 60% of adults in the United States are considered overweight, with about 22% of these being classified as obese.

Diet also contributes to elevated plasma levels of cholesterol, including non-HDL cholesterol. Non-HDL cholesterol is associated with atherogenesis and its sequalea including arteriosclerosis, myocardial infarction, ischemic stroke, and other forms of heart disease that together rank as the most prevalent type of illness in industrialized countries. Indeed, an estimated 12 million people in the United States suffer with coronary artery disease and about 36 million require treatment for elevated cholesterol levels.

With the high prevalence of diabetes, obesity, and cholesterol-related conditions, there remains a need for approaches that treat one or more of these conditions, including reducing unwanted side effects. The present invention provides methods, compositions, and kits for using phospholipase inhibitors to treat phospholipase-related conditions, such as insulin-related conditions (e.g., diabetes), weight-related conditions (e.g., obesity) and/or cholesterol-related conditions.

Accordingly, there remains a need in the art for more beneficial phospholipase inhibitor compositions, methods of using such compositions, and treatments involving such compositions.

### SUMMARY OF THE INVENTION

One first aspect of the present invention relates to a composition comprising a phospholipase inhibitor. In preferred embodiments of this first aspect of the invention, the phospholipase inhibitor is a multivalent phospholipase inhibitor—having two or more phospholipase inhibiting moieties linked with each other, preferably covalent linked with each other, for example through one or more linking moieties, optionally also through one or more multifunctional bridge moieties. Generally, for example, the multivalent phospholipase inhibitors of this first aspect of the invention can be represented by the formula D-I 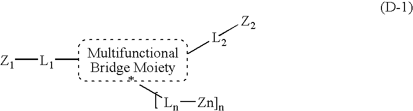
where L is generally a linking moiety, and Z is generally a phospholipase inhibiting moiety. The multifunctional bridge moiety can be a polymer or an oligomer or a non-repeating moiety, in each case having two or more, and preferably at least (n+2), reactive sites to which the two or more phospholipase inhibiting moieties are bonded, preferably covalently bonded. The polymer or oligomer moiety can comprise repeat units consisting of a repeat moiety selected from alkyl (e.g., —CH₂—), substituted alkyl (e.g., —CHR—), alkenyl, substituted alkenyl, alkynyl, substituted alkynyl, phenyl, aryl, heterocyclic, amine, ether, sulfide, disulfide, hydrazine, and any of the foregoing substituted with oxygen, sulfur, sulfonyl, phosphonyl, hydroxyl, alkoxyl, amine, thiol, ether, carbonyl, carboxyl, ester, amide, alkyl, alkenyl, alkynyl, aryl, heterocyclic, as well as moieties comprising combinations thereof. Further and preferred polymer and oligomer moieties are described hereinafter. Generally, a non-repeating multifunctional bridge moiety can be a moiety selected from alkyl, phenyl, aryl, alkenyl, alkynyl, heterocyclic, amine, ether, sulfide, disulfide, hydrazine, and any of the foregoing substituted with oxygen, sulfur, sulfonyl, phosphonyl, hydroxyl, alkoxyl, amine, thiol, ether, carbonyl, carboxyl, ester, amide, alkyl, alkenyl, alkynyl, aryl, heterocyclic, and moieties comprising combinations thereof (in each permutation). A non-repeating moiety can include repeating units (e.g., methylene) within portions or segments thereof without repeating as a whole. In some embodiments, the integer n most preferably ranges from 0 to 10 (such that the number of phospholipase inhibitor moieties ranges from 2 to 12) or from 1 to 10 (such that the number of phospholipase inhibitor moieties ranges from 3 to 12). In embodiments with n ranging from 0 to 10 or from 1 to 10, the multifunctional bridge moiety may be preferred to be an oligomer moiety or a non-repeating moiety. In alternative embodiments, n can more generally range from 0 to about 500, or from 1 to about 500, preferably from 0 to about 100, or from 1 to about 100, and more preferably from 0 to about 50, or from 1 to about 50, and even more preferably from 0 to about 20, or from 1 to about 20. In some particular embodiments, the number of phospholipase inhibiting moieties can be lower, ranging for example from 2 to about 10 (with the integer n correspondingly ranging from 0 to about 8), or from 3 to about 10 (correspondingly with n ranging from 1 to about 8). In some other embodiments, the number of phospholipase inhibiting moieties can range from 2 to about 6 (correspondingly with n ranging from 0 to about 4), or from 3 to about 6 (correspondingly with n ranging from 1 to about 4). In certain embodiments, the number of phospholipase inhibiting moieties can range from 2 to 4 (correspondingly with n ranging from 0 to 2), or from 3 to 4 (correspondingly with n ranging from 1 to 2).

In a first preferred embodiment within the first aspect of the invention, the phospholipase inhibitor is defined by [claim 1].

In a second preferred embodiment within the first aspect of the invention, the phospholipase inhibitor is defined by [claim 2].

In a third preferred embodiment within the first aspect of the invention, the phospholipase inhibitor is defined by [claim 3].

In a fourth preferred embodiment within the first aspect of the invention, the phospholipase inhibitor is defined by [claim 4].

In a second aspect, the invention relates to a composition of matter comprising a substituted organic compound or a salt thereof, the substituted organic compound being represented by a formula selected from one or more of those set forth in [claim 38].

In some embodiments of this second aspect of the invention, the compound (or salt) of the second aspect of the invention can be a phospholipase inhibiting moiety for application in connection with the first aspect of the invention, including preferred embodiments thereof.

In preferred embodiments, including the first, second, third and fourth embodiments of the first aspect of the invention, as well as all embodiments of the second aspect of the invention, the phospholipase inhibitor can be adapted such that (following administration to a subject) the phospholipase inhibitor is localized in a gastrointestinal lumen. In some embodiments included within a first general approach of these embodiments of the invention, the inhibitor is not absorbed through a gastrointestinal mucosa. In embodiments included within a second general approach of these embodiments of the invention, the inhibitor is localized in the gastrointestinal lumen as a result of efflux from a gastrointestinal mucosal cell.

Generally, in all embodiments of the invention (including the first aspect or the second aspect of the invention), including for example for embodiments relating to the aforementioned first general approach or second general approach, the inhibitor can have lumen-localization functionality. For example, the phospholipase inhibitor can have chemical and physical properties, such as low permeability (e.g., across biological membranes) that impart lumen-localization functionality to the inhibitor. Preferably, the inhibitors of these embodiments can additionally or alternatively have other chemical and/or physical properties such that at least about 80% of the phospholipase inhibitor remains in the gastrointestinal lumen, and preferably at least about 90% of the phospholipase inhibitor remains in the gastrointestinal lumen (in each case, following administration of the inhibitor to the subject). Such chemical and/or physical properties can be realized, for example, by an inhibitor comprising at least one moiety selected from an oligomer moiety, a polymer moiety, a hydrophobic moiety, a hydrophilic moiety, a charged moiety and combinations thereof. These embodiments can be used in various and specific combination, and in each permutation, with other aspects and embodiments described above or below herein.

Generally, in embodiments of the first and second aspects of the invention, including for example for embodiments relating to the first general approach or second general approach, the inhibitor can have enzyme-inhibiting functionality. Generally, in embodiments of the invention, including for example for embodiments relating to the first general approach or second general approach thereof, the phospholipase inhibitor can comprise or consist essentially of a small substituted organic molecule, an oligomer, a polymer, moieties of any thereof, and combinations of any of the foregoing. In some embodiments, the phospholipase inhibitor can comprise a phospholipase inhibiting moiety linked (e.g., covalently linked, directly or indirectly using a linking moiety) to a non-absorbed or non-absorbable moiety, preferably to a multivalent moiety such as set forth in connection with the first aspect of the invention or more generally for example, to a non-absorbed or non-absorbable oligomer or polymer moiety. In these embodiments, the phospholipase inhibiting moiety can be, for example, a moiety of a small substituted organic molecule having inhibiting functionality. These embodiments can be used in various and specific combination, and in each permutation, with other aspects and embodiments described above or below herein.

Generally, embodiments of the first aspect of the invention or the second aspect of the invention can further comprise oligomers or polymers (or moieties thereof) bonded, preferably covalently bonded, to the substituted organic compounds or salts thereof, in particular where such compounds or salts thereof are phospholipase inhibiting moieties. The oligomers or polymers can be specifically configured and can be adapted to contribute to lumen-localization functionality and/or to enzyme-inhibiting functionality of the phospholipase inhibitor. The oligomer (or oligomer moiety) or the polymer (or polymer moiety): can generally be soluble or insoluble; can generally be a cross-linked oligomer (or oligomer moiety) or a cross-linked polymer (or polymer moiety); can generally be a homopolymer or a copolymer (including polymers having two monomer-repeat-units, terpolymers and higher-order polymers), including for example random copolymer moieties and block copolymer moieties; can generally include one or more ionic monomer moieties such as one or more anionic monomer moieties; can generally include one or more hydrophobic monomer moieties; can generally include one or more hydrophilic monomer moieties; and can generally include any of the foregoing features in combination. Particularly preferred embodiments of oligomers or polymers (or moieties thereof) are further described hereinafter in the context of independent aspects of the invention, but are equally applicable and are specifically contemplated as being applicable in conjunction with this second aspect of the invention (as well, for example, including both the first and second general approaches for lumen-localization). These embodiments can be used in various and specific combination, and in each permutation, with other aspects and embodiments described above or below herein.

Generally, in embodiments comprising a small substituted organic molecule (or a moiety thereof) as a phospholipase inhibitor (or as a phospholipase inhibiting moiety)—including embodiments with inhibitors comprising a phospholipase inhibiting moiety linked to a non-absorbed or non-absorbable moiety such as an oligomer or polymer moiety, the small molecule inhibitor or inhibiting moiety can be a known or future-discovered small molecule having phospholipase inhibiting activity. In some preferred embodiments, the small molecule phospholipase inhibitor or inhibiting moiety can comprise a moiety of a substituted organic compound having a fused five-member ring and six-member ring, and preferably a fused five-member ring and six-member ring having one or more heteroatoms (e.g., nitrogen, oxygen) substituted within the ring structure of the five-member ring, within the ring structure of the six-member ring, or within the ring structure of each of the five-member and six-member rings, and in each case with substituent groups effective for imparting phospholipase inhibiting functionality to the moiety. Preferably, such substituent groups are also effective for imparting lumen-localizing functionality to the moiety. In preferred embodiments, a small molecule phospholipase inhibitor or inhibiting moiety can comprise an indole-containing moiety (referred to herein interchangeably as an indole-moiety), such as a substituted indole moiety. In some embodiments, the phospholipase inhibitor or inhibiting moiety can be a phospholipid analog or a transition state analog. In some embodiments, the small molecule inhibitor or inhibiting moiety can further comprise at least one substituent having functionality for linking directly or indirectly to a non-absorbed or non-absorbable moiety, such as an oligomer or polymer moiety. For example, a phospholipids analog or transition state analog can be linked directly or indirectly to the non-absorbed moiety, for example, via its hydrophobic group. Particularly preferred embodiments of the phospholipase inhibitor or inhibiting moiety are further described hereinafter in the context of independent aspects of the invention, but are equally applicable and are specifically contemplated as being applicable in conjunction with this first aspect of the invention, including both the first and second general approaches thereof. Also, these embodiments can be used in various and specific combination, and in each permutation, with other aspects and embodiments described above or below herein.

Another third aspect of the invention relates to a composition comprising a phospholipase inhibitor (including phospholipase inhibitors within the first aspect of the invention or that include compounds, salts or moieties of the second aspect of the invention), in which the phospholipase inhibitor comprises an oligomer moiety or polymer moiety or non-repeating moiety covalently linked to a phospholipase inhibiting moiety, and in which the phospholipase inhibitor is further characterized by one or more features selected from the group consisting of: (a) the phospholipase inhibitor being stable while passing through at least the stomach, the duodenum and the small intestine of the gastrointestinal tract; (b) the phospholipase inhibitor inhibiting activity of a secreted, calcium-dependent phospholipase present in the gastrointestinal lumen; (c) the phospholipase inhibitor inhibiting activity of a phosholipase-A₂ IB; (d) the phospholipase inhibitor inhibiting activity of a phosholipase-A₂, but essentially does not inhibit other gastrointestinal mucosal membrane-bound phospholipases; (e) the phospholipase inhibitor being insoluble in the fluid phase of the gastrointestinal tract; (f) the phospholipase inhibitor being adapted to associate with a lipid-water interface; (g) the oligomer or polymer moiety comprising at least one monomer that is anionic and at least one monomer that is hydrophobic; (h) the oligomer or polymer moiety being a copolymer moiety, the copolymer moiety being a random copolymer moiety, a block copolymer moiety; a hydrophobic copolymer moiety; and combinations thereof; and (i) combinations thereof, including each permutation of combinations. These features can also be characterizing features of embodiments within first aspect of the invention as described above. Reciprocally, the polymer moiety and/or the phospholipase inhibiting moiety of this second aspect of the invention can themselves be further characterized by features already described above in connection with the first aspect of the invention. These embodiments can be used in various and specific combination, and in each permutation, with other aspects and embodiments described above or below herein.

In some embodiments (relevant to the first aspect of the invention, and also to the second aspect of the invention), the invention is directed to a composition comprising the phospholipase inhibitor, in which the phospholipase inhibitor comprises a repeat unit, an oligomer or a polymer having the formula (A) 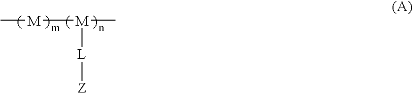
wherein n is an integer, m is an integer (with at least one of which m or n being a non-zero integer), M is a monomer moiety (i.e., a constituent moiety of a polymer) (e.g., each M being independently selected from one or more specific monomer moieties, such as a first monomer moiety, M₁, a second monomer moiety, M₂, a third monomer moiety M₃, a fourth monomer moiety, M₄, etc., where each thereof can be different from each other), L is an optional linking moiety and Z is a phospholipase inhibiting moiety, such as phospholipase inhibitors of the first aspect or the second aspect of the invention. The phospholipase inhibitor preferably comprises an oligomer or a polymer having the formula (A). Embodiments included within this third aspect of the invention can be used in various and specific combination, and in each permutation, with other aspects and embodiments described above or below herein.

In some embodiments (relevant to the first aspect of the invention, and also to the second aspect of the invention), the invention is directed to a composition comprising the phospholipase inhibitor, where the phospholipase inhibitor comprises a compound of the formula (B)

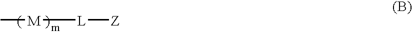

wherein m is a non-zero integer, M is a monomer moiety (e.g., each M being independently selected from one or more specific monomer moieties, such as a first monomer moiety, M₁, a second monomer moiety, M₂, a third monomer moiety M₃, a fourth monomer moiety, M₄, etc., where each thereof can be different from each other), L is an optional linking moiety and Z is a phospholipase inhibiting moiety. The embodiments included within this fourth aspect of the invention can be used in various and specific combination, and in each permutation, with other aspects and embodiments described above or below herein.

In some embodiments (relevant to the first aspect of the invention, and also to the second aspect of the invention), the invention is directed to a composition which can comprise a phospholipase inhibitor, where the phospholipase inhibitor comprises a compound having the formula (C) 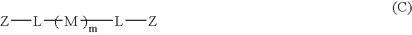
wherein m is a non-zero integer, M is a monomer moiety (e.g., each M being independently selected from one or more specific monomer moieties, such as a first monomer moiety, M₁, a second monomer moiety, M₂, a third monomer moiety M₃, a fourth monomer moiety, M₄, etc., where each thereof can be different from each other), L are each independently selected optional linking moieties and Z are each, independently selected phospholipase inhibiting moieties. Generally, these embodiments included within this fifth aspect of the invention can be used in various and specific combination, and in each permutation, with other aspects and embodiments described above or below herein.

In some embodiments (relevant to the first aspect of the invention, and also to the second aspect of the invention), the invention is directed to a composition comprising a phospholipase inhibitor, which comprises an oligomer or polymer moiety covalently linked to a phospholipase inhibiting moiety, preferably with the phospholipase inhibitor comprising a compound having the formula (C-1) 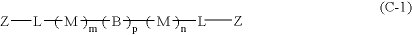
wherein m is a non-zero integer, n is a non-zero integer, p is a non-zero integer, M are each independently selected monomer moieties (e.g., each M being independently selected from one or more specific monomer moieties, such as a first monomer moiety, M₁, a second monomer moiety, M₂, a third monomer moiety M₃, a fourth monomer moiety, M₄, etc., where each thereof can be different from each other), B is a bridging moiety, L are each independently selected optional linking moieties, and Z are each independently selected phospholipase inhibiting moieties. Generally, these embodiments included within this sixth aspect of the invention can be used in various and specific combination, and in each permutation, with other embodiments described above or below herein.

In each of these various embodiments of the invention, the phospholipase inhibitor can be further characterized by one or more features selected from the features described above in connection with the first and/or second aspects of the invention. These embodiments can be used in various and specific combination, and in each permutation, with other aspects and embodiments described above or below herein.

Generally, with respect to any of the aforementioned aspects or following-discussed aspects of the invention, the phospholipase inhibitor can be adapted so that it inhibits activity of a phospholipase, especially and preferably characterized in that the inhibitor: inhibits activity of a secreted, calcium-dependent phospholipase present in the gastrointestinal lumen; inhibits a phospholipase-A₂ present in the gastrointestinal lumen; inhibits activity of secreted, calcium-dependent phospholipase-A₂ present in the gastrointestinal lumen; inhibits activity of phospholipase-A₂ IB present in the gastrointestinal lumen; inhibits a phospholipase A₂, such as phospholipase-A₂ IB, as well as inhibits phospholipase B; and/or combinations thereof. These embodiments can be used in various and specific combination, and in each permutation, with other aspects and embodiments described above or below herein.

Also, with respect to any of the aspects of the invention, the phospholipase inhibitor can be relatively specific or strictly specific, for example, including having activity for inhibiting a phospholipase-A₂, such as a phospholipase-A₂ IB, but where the phospholipase inhibitor essentially does not inhibit one or more other enzymes, as follows: essentially does not inhibit a lipase; essentially does not inhibit phospholipase-B; essentially does not inhibit other gastrointestinal phospholipases having activity for catabolizing a phospholipids; essentially does not inhibit other gastrointestinal phospholipases having activity for catabolizing phosphatidylcholine or phosphatidylethanolamine; and/or essentially does not inhibit other gastrointestinal mucosal membrane-bound phospholipases, and combinations thereof. In some embodiments, the inhibitor does not act on the gastrointestinal mucosa. These embodiments can be used in various and specific combination, and in each permutation, with other aspects and embodiments described above or below herein.

Generally, in the embodiments included within any of the aspects of the invention, the phospholipase inhibitors herein can be characterized in that they produce a therapeutic and/or a prophylactic benefit in treating an insulin-related condition (e.g., diabetes type 2), a weight-related condition (e.g., obesity), a cholesterol-related condition (e.g., hypercholesterolemia), and combinations thereof, in each case in a subject receiving said inhibitor.

Another fourth aspect of the invention provides methods of using a composition comprising a phospholipase inhibitor (including, for example, any of the phospholipase inhibitors included within the first through seventh aspects of the invention). Generally, the method comprises inhibiting a phospholipase by administering an effective amount of the composition to a subject in need thereof. In some embodiments, the method comprises specifically or selectively inhibiting a phospholipase (e.g., with various aspects of specificity being as described above). These method embodiments can be used in various and specific combination, and in each permutation, with other aspects and embodiments described above or below herein.

In another fifth aspect, the invention is directed to method of treating a condition comprising administering an effective amount of a phospholipase inhibitor to a subject, and localizing the inhibitor in a gastrointestinal lumen such that upon administration to the subject, essentially all of the phospholipase inhibitor remains in the gastrointestinal lumen. In preferred embodiments, this aspect of the invention can include, in one preferred approach, a method of treating a condition comprising administering an effective amount of a phospholipase-A₂ inhibitor to a subject, the phospholipase-A₂ inhibitor preferably being a phospholipase-A₂ IB inhibitor, and in any case, the phospholipase-A₂ inhibitor being localized in a gastrointestinal lumen upon administration to the subject. This aspect of the invention can also include, in a second preferred approach, a method for modulating the metabolism of fat, glucose or cholesterol in a subject, the method comprising administering an effective amount of a phospholipase-A₂ inhibitor to the subject, the phospholipase-A₂ inhibitor inhibiting activity of a secreted, calcium-dependent phospholipase-A₂ present in a gastrointestinal lumen, the phospholipase inhibitor being localized in the gastrointestinal lumen upon administration to the subject. Preferably, and generally, the embodiments of this method can include treating a condition by administering an effective amount of a phospholipase inhibitor to a subject in need thereof where the inhibitor is not absorbed through a gastrointestinal mucosa and/or where the inhibitor is localized in a gastrointestinal lumen as a result of efflux from a gastrointestinal mucosal cell. Such phospholipase inhibitors can be used in the treatment of phospholipase-related conditions, preferably phospholipase A₂-related conditions and phospholipase A₂-related conditions induced by diet. Preferably, the condition treated is an insulin-related condition (e.g., diabetes type 2), a weight-related condition (e.g., obesity), a cholesterol-related condition (e.g., hypercholesterolemia), and combinations thereof. These embodiments can be used in various and specific combination, and in each permutation, with other aspects and embodiments described above or below herein.

In a related sixth aspect, the invention is directed to medicament comprising a phospholipase-A₂ inhibitor for use as a pharmaceutical. The phospholipase-A₂ inhibitor of the medicament can preferably be localized in a gastrointestinal lumen upon administration of the medicament to a subject. Preferably, the medicament comprises a phospholipase-A₂ IB inhibitor. These embodiments can be used in various and specific combination, and in each permutation, with other aspects and embodiments described above or below herein.

In another seventh aspect, the invention is directed to a method comprising use of a phospholipase-A₂ inhibitor for manufacture of a medicament for use as a pharmaceutical, where the phospholipase-A₂ inhibitor is localized in a gastrointestinal lumen upon administration of the medicament to a subject. Preferably, the medicament is manufactured using a phospholipase-A₂ IB inhibitor. These embodiments can be used in various and specific combination, and in each permutation, with other aspects and embodiments described above or below herein.

A further eighth aspect of the invention is directed to a food product composition comprising an edible foodstuff and a phospholipase inhibitor (such as a phospholipase-A₂ inhibitor) where the phospholipase inhibitor (or phospholipase-A₂ inhibitor) is localized in a gastrointestinal lumen upon ingestion of the food product composition. Preferably, the foodstuff comprises a phospholipase-A₂ IB inhibitor. In some embodiments, the foodstuff can comprise (or can consist essentially of) a vitamin supplement and a phospholipase inhibitor. These embodiments can be used in various and specific combination, and in each permutation, with other aspects and embodiments described above or below herein.

Generally, and preferably in connection with any of the fourth through eighth aspects of the invention, the phospholipase-A₂ inhibitor does not induce substantial steatorrhea following administration or ingestion thereof. These embodiments can be used in various and specific combination, and in each permutation, with other aspects and embodiments described above or below herein.

Those of skill in the art will recognize that the compounds described herein may exhibit the phenomena of tautomerism, conformational isomerism, geometric isomerism and/or optical isomerism. It should be understood that the invention encompasses any tautomeric, conformational isomeric, optical isomeric and/or geometric isomeric forms of the compounds having one or more of the utilities described herein, as well as mixtures of these various different forms. Prodrugs and active metabolites of the compounds described herein are also within the scope of the present invention.

Although various features are described above to provide a summary of various aspects of the invention, it is contemplated that many of the details thereof as described below can be used with each of the various aspects of the invention, without limitation. Other features, objects and advantages of the present invention will be in part apparent to those skilled in art and in part pointed out hereinafter. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

FIG. 1A through FIG. 1D are schematic representations illustrating: (i) interaction of a phospholipase with a lipid-water interface (FIG. 1A); (ii) interaction of a non-absorbed phospholipase inhibitor with a lipid-water interface (FIG. 1B); (iii) interaction of a non-absorbed phospholipase inhibitor with the phospholipase enzyme (FIG. 1C); and (iv) interaction of a non-absorbed phospholipase inhibitor with both a lipid-water interface and with the phospholipase enzyme (FIG. 1D).

FIG. 2 is a schematic representation illustrating phospholipase inhibitors comprising polymer moieties covalently linked to phospholipase inhibiting moieties (represented schematically by “I*”), where the polymer moieties are shown as being soluble or insoluble, and further illustrating interaction between the phospholipase inhibitors and phospholipase-A₂ in a gastrointestinal fluid in the vicinity of gastrointestinal lipid vesicles.

FIG. 3A through FIG. 3C are schematic representations illustrating phospholipase inhibitors comprising polymer moieties covalently linked to one or more phospholipase inhibiting moiety (represented schematically by “I*”), where (i) the phospholipase inhibitor comprises a hydrophobic polymer moiety, adapted such that the inhibitor associates with a lipid-water interface of a lipid vesicle (shown with the hydrophobic polymer moiety being substantially integral with the lipid bilayer) (FIG. 3A); (ii) the phospholipase inhibitor comprises a polymer moiety having a first hydrophobic block and a second hydrophilic block with the second hydrophilic block being proximal to the phospholipase inhibiting moiety, adapted such that the inhibitor associates with a lipid-water interface of a lipid vesicle (shown with the hydrophobic block being substantially integral with the lipid bilayer and with the hydrophilic block being substantially associated within the aqueous phase surrounding the lipid bilayer) (FIG. 3B); and (iii) the phospholipase inhibitor comprises a hydrophobic polymer moiety covalently linked to two inhibiting moieties, and adapted such that the inhibitor associates with a lipid-water interface of a lipid vesicle (shown with the hydrophobic polymer moiety being substantially integral with and looped through the lipid bilayer (FIG. 3C); and in each case (i), (ii) and (iii) allowing for interaction between the inhibiting moiety and phospholipase-A₂ substantially proximate to the vesicle surface.

FIG. 4 is a schematic representation of a chemical reaction in which phospholipase-A2 enzyme (PLA2) catalyzes hydrolysis of phospholipids to corresponding lysophospholipids.

FIG. 5 is a chemical formula for [2-(3-(2-amino-2-oxoacetyl)-1-(biphenyl-2-ylmethyl)-2-methyl-1H-indol-4-yloxy)acetic acid], also referred to herein as ILY-4001 and as methyl indoxam.

FIGS. 6A through 6D are schematic representations including chemical formulas illustrating indole compounds (FIG. 6A, FIG. 6C and FIG. 6D) and indole-related compounds (FIG. 6B).

FIG. 7 is a schematic illustration, including chemical formulas, which outlines the overall synthesis scheme for ILY-4001 [2-(3-(2-amino-2-oxoacetyl)-1-(biphenyl-2-ylmethyl)-2-methyl-1H-indol-4-yloxy)acetic acid] as described in Example 1A.

FIGS. 8A and 8B are a schematic representation (FIG. 8A) of an in-vitro fluorometric assay for evaluating PLA2 IB enzyme inhibition, and a graph (FIG. 8B) showing the results of Example 6A in which the assay was used to evaluate ILY-4001 [2-(3-(2-amino-2-oxoacetyl)-1-(biphenyl-2-ylmethyl)-2-methyl-1H-indol-4-yloxy)acetic acid].

FIGS. 9A and 9B are graphs showing the results from the in-vitro Caco-2 permeability study of Example 6B for ILY-4001 [2-(3-(2-amino-2-oxoacetyl)-1-(biphenyl-2-ylmethyl)-2-methyl-1H-indol-4-yloxy)acetic acid] (FIG. 9A) and for Lucifer Yellow and Propranolol as paracellular and transcellular transport controls (FIG. 9B).

FIG. 10 is a schematic illustration, including chemical formulas, which outlines the overall synthesis scheme to prepare 3-(3-aminooxalyl-1-biphenyl-2-yl methyl-4-carboxymethoxy-2-methyl-1H-indol-5-yl)-propionic acid as described in Example 1C.

FIG. 11 is a schematic illustration, including chemical formulas, which outlines the overall synthesis scheme for preparing a polymer-linked ILY-4001—namely, a random copolymer of [3-Aminooxalyl-2-methyl-1-(2′-vinyl-biphenyl-2-ylmethyl)-1H-indol-4-yloxy]-acetic acid, styrene, and styrene sulfonic acid sodium salt, as described in Example 1D.

FIG. 12 is a schematic illustration, including chemical formulas, which outlines the overall synthesis scheme by which ILY-4001 can be provided with linking groups to form [3-Aminooxalyl-2-methyl-1-(4-vinyl-benzyl)-1H-indol-4-yloxy]-acetic acid (21); Synthesis of (1-Acryloyl-3-aminooxalyl-2-methyl-1H-indol-4-yloxy)-acetic acid (23); Synthesis of {3-Aminooxalyl-2-methyl-1-[2-(pyrazole-1-carbothioylsulfanyl)propionyl]-1H-indol-4-yloxy}-acetic acid (26), as described in Example 2.

FIGS. 13A through 13D are graphs summarizing the results of an in-vivo study of Example 10, including: a graph illustrating the results of Example 10A, showing body weight gain in groups of mice receiving ILY-4001 at low dose (4001-L) and high dose (4001-H) as compared to wild-type control group (Control) and as compared to genetically deficient PLA2 (−/−) knock-out mice (PLA2 KO) (FIG. 13A); a graph illustrating the results of Example 10B, showing fasting serum glucose levels in groups of mice receiving ILY-4001 at low dose (4001-L) and high dose (4001-H) as compared to wild-type control group (Control) and as compared to genetically deficient PLA2 (−/−) knock-out mice (PLA2 KO) (FIG. 13B); and graphs illustrating the results of Example 10C, showing serum cholesterol levels (FIG. 13C) and serum triglyceride levels (FIG. 13D) in groups of mice receiving ILY-4001 at low dose (4001-L) and high dose (4001-H) as compared to wild-type control group (Control) and as compared to genetically deficient PLA2 (−/−) knock-out mice (PLA2 KO).

FIGS. 14A, 14B, 14C and 14D are graphs depicting results for Test Article ILY4008 (ILY-V-26) in a C57BL/6J mouse model of obesity.

FIGS. 15A, 15B, 15C and 15D are graphs depicting results for Test Article ILY4011 (ILY-V-30) in a C57BL/6J mouse model of obesity.

FIGS. 16A, 16B and 16C are graphs depicting results for Test Article ILY4013 (ILY-V-32) in a C57BL/6J mouse model of obesity.

FIGS. 17A, 17B, and 17C are graphs depicting results for Test Article ILY4016 (ILY-IV-40) in a C57BL/6J mouse model of obesity.

FIGS. 18A, 18B, 18C, 18D, 18E and 18F are graphs depicting results for Test Article ILY4008 (ILY-V-26) in a LDL receptor knockout mouse model.

FIGS. 19A, 19B, 19C, 19D, 19E and 19F are graphs depicting results for Test Article ILY4011 (ILY-V-30) in a LDL receptor knockout mouse model.

FIGS. 20A, 20B, 20C and 20D are graphs depicting results for Test Article ILY4013 (ILY-V-32) in a LDL receptor knockout mouse model.

FIGS. 21A, 21B, 21C and 21D are graphs depicting results for Test Article ILY4016 (ILY-IV-40) in a LDL receptor knockout mouse model.

FIGS. 22A, 22B, 22C, 22D and 22E are graphs depicting results for Test Article ILY4008 (ILY-V-26) in a NONcNZO10/LtJ mouse model of Type II diabetes.

FIGS. 23A, 23B, 23C, 23D and 23E are graphs depicting results for Test Article ILY4011 (ILY-V-30) in a NONcNZO10/LtJ mouse model of Type II diabetes.

FIGS. 24A, 24B, 24C, 24D and 24E are graphs depicting results for Test Article ILY4013 (ILY-V-32) in a NONcNZO10/LtJ mouse model of Type II diabetes.

FIGS. 25A, 25B, 25C, 25D and 25E are graphs depicting results for Test Article ILY4016 (ILY-IV-40) in a NONcNZO10/LtJ mouse model of Type II diabetes.

FIGS. 26A and 26B are graphs depicting results for Test Article ILY4016 (ILY-IV-40), Test Article ILY4008 (ILY-V-26), Test Article ILY4013 (ILY-V-32), Test Article ILY4011 (ILY-V-30), and Test Article ILY4017 (ILY-V-37) in a hamster diet-induced dyslipidemia model.

### DETAILED DESCRIPTION OF THE INVENTION

The present invention provides phospholipase inhibitors, compositions (including pharmaceutical formulations, medicaments and foodstuffs) comprising such phospholipase inhibitors, and methods for identifying, making and using such phospholipase inhibitors and compositions, including use thereof as pharmaceuticals for treatments of various conditions. The phospholipase inhibitors of the present invention can find use in treating a number of phospholipase-related conditions, including insulin-related conditions (e.g., diabetes), weight-related conditions (e.g., obesity), cholesterol-related disorders and any combination thereof, as described in detail below.

Generally, the phospholipase inhibitors of the invention should be adapted for having both lumen-localization functionality as well as enzyme-inhibition functionalization. In some schema, certain aspects of such dual functionality can be achieved synergistically (e.g., by using the same structural features and/or charge features); in other schema, the lumen-localization functionality can be achieved independently (e.g., using different structural and/or charge features) from the enzyme-inhibition functionality.

Overview

The phospholipase inhibitors of the present invention are (in one aspect) multivalent phospholipase inhibitors. Multivalent inhibitors can be advantageous with respect to lumen-localization, because they are generally physically of larger dimension and generally have a larger molecular weight than monovalent (e.g., small molecule) phospholipase inhibitors. Interestingly and unexpectedly, and without being bound by theory or to performance criteria not specifically recited in the claims, the activity (e.g., IC50) of multivalent phospholipase inhibitors can be comparable to or can exceed, on a per weight basis, the activity of monovalent (e.g., small molecule) phospholipase inhibitors. This is particularly surprising in view of the accepted wisdom within the art of phospholipase inhibitors, in which it is generally recognized to be little physical space for altering the dimensions of an inhibitor, since the inhibitor is thought to be active in a position situated between the enzyme and the bilipidic bilayer.

The compounds (or salts thereof) of the second aspect of the invention are useful as phospholipase inhibitors or as phospholipase inhibiting moieties. In one example, for instance, the compounds (or salts thereof) or moieties derived therefrom (having one or more functionalized groups) can be used in connection with the first aspect of the invention to form multivalent phospholipase inhibitors.

The phospholipase inhibitors are, in any aspect or embodiment, preferably localized in the gastrointestinal lumen, such that upon administration to a subject, the phospholipase inhibitors remain substantially in the gastrointestinal lumen. Following administration, the localized phospholipase inhibitors can remain in and pass naturally through the gastrointestinal tract, including the stomach, the duodenum, the small intestine and the large intestine (until passed out of the body via the gastrointestinal tract). The phospholipase inhibitors are preferably substantially stable (e.g., with respect to composition and/or with respect to functionality for inhibiting phospholipase) while passing through at least the stomach and the duodenum, and more preferably, are substantially stable while passing through the stomach, the duodenum and the small intestine of the gastrointestinal tract, and most preferably, are substantially stable while passing through the entire gastrointestinal tract. The phospholipase inhibitors can act in the gastrointestinal lumen, for example to catabolize phospholipase substrates or to modulate the absorption and/or downstream activities of products of phospholipase digestion.

In the present invention, phospholipase inhibitors are localized within the gastrointestinal lumen, in one approach, by being not absorbed through a gastrointestinal mucosa. In some embodiments, the phospholipase inhibitors of the present invention can be localized in a gastrointestinal lumen and can also be cell impermeable, e.g., not internalized into a cell. As another approach, the phospholipase inhibitors can be localized in the gastrointestinal lumen by being absorbed into a mucosal cell and then effluxed back into a gastrointestinal lumen. Hence, in some embodiments, the phospholipase inhibitors are cell permeable, e.g., can be internalized into a cell, and are also localized in a gastrointestinal lumen. In these embodiments, gastrointestinal localization can be facilitated by an efflux mechanism. Each of these general approaches for achieving gastrointestinal localization is further described below.

Generally, without being constrained by categorization into one or more of the aforementioned general approaches by which the phospholipase inhibitor can be lumen-localized, preferred phospholipase inhibitors of the invention (as contemplated in the various aspects of the invention) can be realized by several general embodiment formats—suitable generally with the first or second aspect of the invention. In one general embodiment, for example, the phospholipase inhibitor can consist essentially of an oligomer or a polymer. In another embodiment, the phospholipase inhibitor can comprise an oligomer or polymer moiety covalently linked, directly or indirectly through a linking moiety, to a phospholipase inhibiting moiety, such as a substituted small organic molecule moiety. In a further general embodiment, the phospholipase inhibitor can itself be a substituted small organic molecule. Each of these general embodiments is described below in further detail.

In general for each various embodiments included within the various aspects of the invention, the inhibitor is localized, upon administration to a subject, in the gastrointestinal lumen of the subject, such as an animal, and preferably as a mammal, including for example a human as well as other mammals (e.g., mice, rats, rabbits, guinea pigs, hamsters, cats, dogs, porcine, poultry, bovine and horses). The term “gastrointestinal lumen” is used interchangeably herein with the term “lumen,” to refer to the space or cavity within a gastrointestinal tract, which can also be referred to as the gut of the animal. In some embodiments, the phospholipase inhibitor is not absorbed through a gastrointestinal mucosa. “Gastrointestinal mucosa” refers to the layer(s) of cells separating the gastrointestinal lumen from the rest of the body and includes gastric and intestinal mucosa, such as the mucosa of the small intestine. In some embodiments, lumen localization is achieved by efflux into the gastrointestinal lumen upon uptake of the inhibitor by a gastrointestinal mucosal cell. A “gastrointestinal mucosal cell” as used herein refers to any cell of the gastrointestinal mucosa, including, for example, an epithelial cell of the gut, such as an intestinal enterocyte, a colonic enterocyte, an apical enterocyte, and the like. Such efflux achieves a net effect of non-absorbedness, as the terms, related terms and grammatical variations, are used herein.

Generally, in all embodiments included within the various aspects of the invention, phospholipase inhibitors of the present invention can modulate or inhibit (e.g., blunt or reduce) the catalytic activity of phospholipases, preferably phospholipases secreted or contained in the gastrointestinal tract, including the gastric compartment, and more particularly the duodenum and/or the small intestine. For example, such enzymes include, but are not limited to, secreted Group IB phospholipase A₂ (PL A₂-IB), also referred to as pancreatic phospholipase A₂ (p-PL A₂) and herein referred to as “PL A₂ IB” or “phospholipase-A₂ IB;” secreted Group IIA phospholipase A₂ (PL A₂ IIA); phospholipase A1 (PLA₁); phospholipase B (PLB); phospholipase C (PLC); and phospholipase D (PLD). The inhibitors of the invention preferably inhibit the activity at least the phospholipase-A₂ IB enzyme.

In some embodiments, the inhibitors of the present invention are specific, or substantially specific for inhibiting phospholipase activity, such as phospholipase A₂ activity (including for example phospholipase-A₂ IB). For example, in some preferred embodiments inhibitors of the present invention do not inhibit or do not significantly inhibit or essentially do not inhibit lipases, such as pancreatic triglyceride lipase (PTL) and carboxyl ester lipase (CEL). In some preferred embodiments, inhibitors of the present invention inhibit PL A₂, and preferably phospholipase-A₂ IB, but in each case do not inhibit or do not significantly inhibit or essentially do not inhibit any other phospholipases; in some preferred embodiments, inhibitors of the present invention inhibit PL A₂, and preferably phospholipase-A₂ IB, but in each case do not inhibit or do not significantly inhibit or essentially do not inhibit PLA₁; in some preferred embodiments, inhibitors of the present invention inhibit PL A₂, and preferably phospholipase-A₂ IB, but do not inhibit or do not significantly inhibit or essentially do not inhibit PLB. In some embodiments, the phospholipase inhibitor does not act on the gastrointestinal mucosa, for example, it does not inhibit or does not significantly inhibit or essentially does not inhibit membrane-bound phospholipases.

The different activities of PL A₂, PL A₁, and PLB are generally well-characterized and understood in the art. PL A₂ hydrolyzes phospholipids at the sn-2 position liberating 1-acyl lysophospholipids and fatty acids; PL A₁ acts on phospholipids at the sn-1 position to release 2-acyl lysophospholipids and fatty acids; and phospholipase B cleaves phospholipids at both sn-1 and sn-2 positions to form a glycerol and two fatty acids. See, e.g., Devlin, Editor, Textbook of Biochemistry with Clinical Correlations, 5^{th} ed. Pp 1104-1110 (2002).

Phospholipids substrates acted upon by gastrointestinal PL A₁, PL A₂ (including phospholipase-A₂ IB) and PLB are mostly of the phosphatidylcholine and phosphatidylethanolamine types, and can be of dietary or biliary origin, or may be derived from being sloughed off of cell membranes. For example, in the case of phosphatidylcholine digestion, PL A₁ acts at the sn-1 position to produce 2-acyl lysophosphatidylcholine and free fatty acid; PL A₂ acts at the sn-2 position to produce 1-acyl lysophosphatidylcholine and free fatty acid; while PLB acts at both positions to produce glycerol 3-phosphorylcholine and two free fatty acids (Devlin, 2002).

Pancreatic PL A₂ (and phospholipase-A₂ IB) is secreted by acinar cells of the exocrine pancreas for release in the duodenum via pancreatic juice. PL A₂ (and phospholipase-A₂ IB) is secreted as a proenzyme, carrying a polypeptide chain that is subsequently cleaved by proteases to activate the enzyme's catalytic site. Documented structure-activity-relationships (SAR) for PL A₂ isozymes illustrate a number of common features (see for instance, Gelb M., Chemical Reviews, 2001, 101:2613-2653; Homan, R., Advances in Pharmacology, 1995, 12:31-66; and Jain, M. K., Intestinal Lipid Metabolism, Biology, pathology, and interfacial enzymology of pancreatic phospholipase A₂, 2001, 81-104, each incorporated herein by reference).

The inhibitors of the present invention can take advantage of certain of these common features to inhibit phospholipase activity and especially PL A₂ activity. Common features of PL A₂ enzymes include sizes of about 13 to about 15 kDa; stability to heat; and 6 to 8 disulfides bridges. Common features of PL A₂ enzymes also include conserved active site architecture and calcium-dependent activities, as well as a catalytic mechanism involving concerted binding of His and Asp residues to water molecules and a calcium cation, in a His-calcium-Asp triad. A phospholipid substrate can access the catalytic site by its polar head group through a slot enveloped by hydrophobic and cationic residues (including lysine and arginine residues) described in more detail below. Within the catalytic site, the multi-coordinated calcium ion activates the acyl carbonyl group of the sn-2 position of the phospholipid substrate to bring about hydrolysis (Devlin, 2002). In some preferred embodiments, inhibitors of the present invention inhibit this catalytic activity of PL A₂ by interacting with its catalytic site.

PL A₂ enzymes are active for catabolizing phospholipids substrates primarily at the lipid-water interface of lipid aggregates found in the gastrointestinal lumen, including, for example, fat globules, emulsion droplets, vesicles, mixed micelles, and/or disks, any one of which may contain triglycerides, fatty acids, bile acids, phospholipids, phosphatidylcholine, lysophospholipids, lysophosphatidylcholine, cholesterol, cholesterol esters, other amphiphiles and/or other diet metabolites. Such enzymes can be considered to act while “docked” to a lipid-water interface. In such lipid aggregates, the phospholipid substrates are typically arranged in a mono layer or in a bilayer, together with one or more other components listed above, which form part of the outer surface of the aggregate. The surface of a phospholipase bearing the catalytic site contacts this interface facilitating access to phospholipid substrates. This surface of the phospholipase is known as the i-face, i.e., the interfacial recognition face of the enzyme. The structural features of the i-face of PL A₂ have been well documented. See, e.g., Jain, M. K, et al, Methods in Enzymology, vol. 239, 1995, 568-614, incorporated herein by reference. The inhibitors of the present invention can take advantage of these structural features to inhibit PL A₂ activity. For instance, it is known that the aperture of the slot forming the catalytic site is normal to the i-face plane. The aperture is surrounded by a first crown of hydrophobic residues (mainly leucine and isoleucine residues), which itself is contained in a ring of cationic residues (including lysine and arginine residues). In some preferred embodiments, inhibitors of the present invention hinder access of PL A₂ to its phospholipid substrates by interacting with this i-face and/or with the lipid-water interface.

In view of the action of phospholipases (e.g. PL A₂) in digesting phospholipid substrates in proximity to the surface of such lipid-aggregates, some embodiments of the invention can involve an approach in which the phospholipase inhibitor associates with a water-lipid interface of a lipid aggregate, thereby allowing for interaction between the inhibitor and phospholipase-A₂ substantially proximal thereto.

Multivalent Phospholipase Inhibitors

The multivalent phospholipase inhibitors of the invention can generally comprise a substituted organic compound or a salt thereof. The substituted organic compound can comprises two or more (or three or more) independently selected phospholipase inhibiting moieties, Z₁, Z₂ . . . Zₙ, (generally referred to as Z) linked through independently selected linking moieties, L₁, L₂ . . . Lₙ, (generally referred to as L) to a multifunctional bridge moiety as represented by formula (D-I) 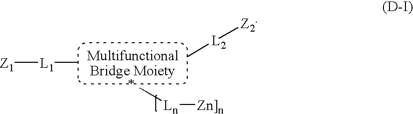
Here, n can be an integer ranging from 0 to 10, or from 1 to 10 in preferred embodiments, such that the number of independently selected phospholipase inhibiting moieties can range from 2 to 12, or from 3 to 12. In alternative embodiments, n can generally range from 0 to about 500, or from 1 to about 500, preferably from 0 to about 100, or from 1 to about 100, and more preferably from 0 to about 50, or from 1 to about 50, and even more preferably from 0 to about 20, or from 1 to about 20. In some embodiments, the number of phospholipase inhibiting moieties can be lower, ranging for example from 2 to about 10 (correspondingly with n ranging from 0 to about 8), or from 3 to about 10 (correspondingly with n ranging from 1 to about 8). In some other embodiments, the number of phospholipase inhibiting moieties can range from 2 to about 6 (correspondingly with n ranging from 0 to about 4), or from 3 to about 6 (correspondingly with n ranging from 1 to about 4). In certain embodiments, the number of phospholipase inhibiting moieties can range from 2 to 4 (correspondingly with n ranging from 0 to 2), or from 3 to 4 (correspondingly with n ranging from 1 to 2).

The two or more phospholipase inhibiting moieties, Z₁, Z₂ . . . Zₙ, can be bonded, preferably covalently bonded, to the multifunctional bridge moiety through the corresponding linking moieties, L₁, L₂ . . . Lₙ, respectively. Preferred phospholipase inhibiting moieties are disclosed hereinafter, and are incorporated in this aspect. Preferred linking moieties are disclosed hereinafter, and are incorporated in this aspect.

In preferred approaches, the multifunctional bridge moiety can have at least (n+2) reactive sites to which the two or more phospholipase inhibiting moieties are bonded. Generally, and preferably, the multifunctional bridge moiety can be selected from the group consisting of alkyl, phenyl, aryl, alkenyl, alkynyl, heterocyclic, amine, ether, sulfide, disulfide, hydrazine, and any of the foregoing substituted with oxygen, sulfur, sulfonyl, phosphonyl, hydroxyl, alkoxyl, amine, thiol, ether, carbonyl, carboxyl, ester, amide, alkyl, alkenyl, alkynyl, aryl, heterocyclic, and moieties comprising combinations thereof. The multifunctional bridge moiety can be an polymer moiety or a oligomer moiety or a non-repeating moiety.

Examples of preferred multifunctional bridge moieties include, for example, sulfide moieties, disulfide moieties, amine moieties, aryl moieties, alkoxyl moieties, etc. Particularly preferred multifunctional bridge unit can be represented by a formula selected from 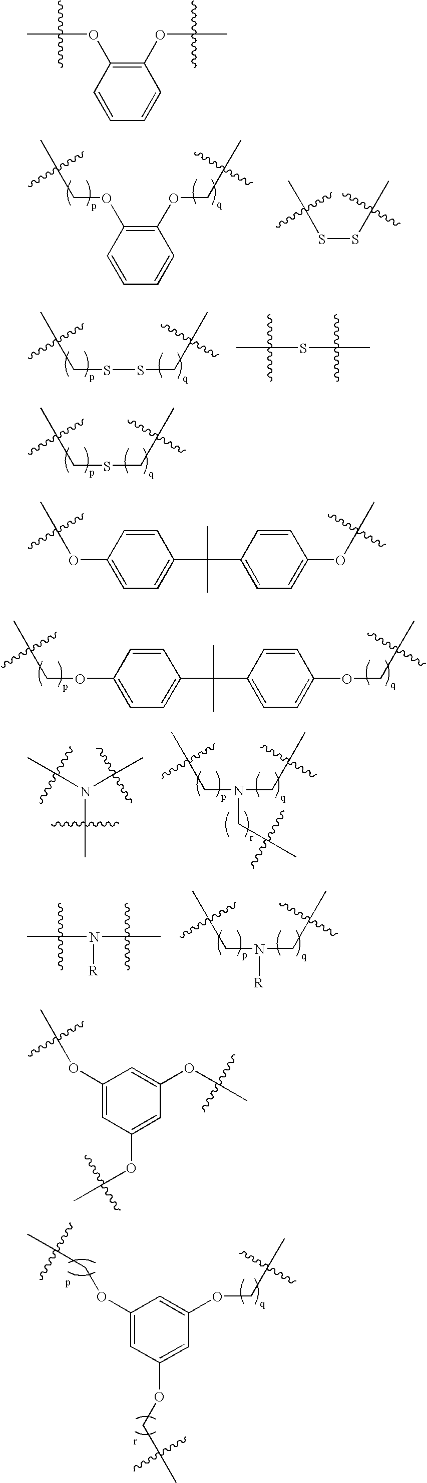
with each p, q and r each being an independently selected integer ranging from 0 to about 48, preferably from 0 to about 36, or from 0 to about 24, or from 0 to about 16. In some embodiments, each p, q and r can be an independently selected integer ranging from 0 to 12. R can be a substituent moiety. The substituent moiety can generally be selected from halide, hydroxyl, amine, thiol, ether, carbonyl, carboxyl, ester, amide, carbocyclic, heterocyclic, and moieties comprising combinations thereof.

In some embodiments, the invention can be a composition comprising a multivalent phospholipase inhibitor compound or salt thereof. The phospholipase inhibitor can comprising a substituted organic compound, or a salt thereof, the substituted organic compound comprising two or more independently selected phospholipase inhibiting moieties, Z₁, Z₂, joined by a linking moiety, L, as represented by the formula (D-I-A) 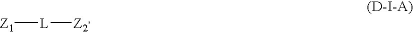
with each of the two or more phospholipase inhibiting moieties being bonded, preferably covalently bonded, to the linking moiety.

In a preferred approach for such embodiments, at least one and preferably each linking moiety, L, has a linker length of at least twenty atoms in the shortest chain through which the two or more phospholipase inhibiting moieties, Z₁, Z₂, are joined. The presence of a carbocyclic ring or heterocyclic ring within the linking moiety, L, counts as a whole number of atoms most closely approximating the calculated diameter of the carbocyclic ring or heterocyclic ring. For example, a benzene ring within the linker sequence can count as two (2) atoms with respect to linker length.

In preferred embodiments within the second general embodiment of the first aspect of the invention, the linking moiety, L, can be a linking moiety represented by the formula selected from (D-II), (D-III) and (D-IV) 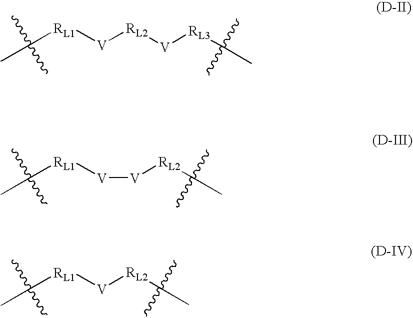
with in each case independently, and as applicable, R_{L1}, R_{L2} and R_{L3} can each be a moiety independently selected from the group consisting of alkyl, substituted alkyl, alkenyl, substituted alkenyl, alkynyl, carbocyclic, heterocyclic, poly(ethylene oxyl), and polyester. In some embodiments, each R_{L1}, R_{L2} and R_{L3} can be an independently selected non-repeating moiety (e.g., a moiety other than an oligomer or polymer) and can be an independently selected from the group consisting of alkyl, substituted alkyl, alkenyl, substituted alkenyl, alkynyl, carbocyclic, heterocyclic. In these embodiments, V can be a multifunctional bridging moiety as generally and specifically described herein. V can be a moiety independently selected from the group consisting of N, O, S, disulfide, carbonyl, ester, amide, urethane, urea, hydrazine, alkene, and alkyne.

For example, in some preferred embodiments, the linking moiety, L, can be a linking moiety represented by the formula selected from (D-II-A), (D-III-A) and (D-IV-A) 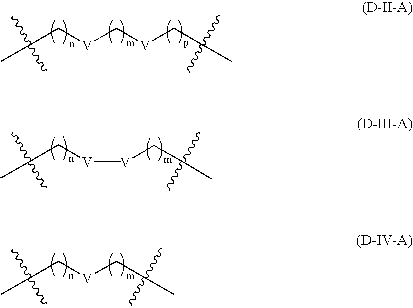
with in each case independently, and as applicable, n, m and p are each independently selected non-zero integers. The integers n, m and p can each be independently selected as ranging from 1 to 50, preferably from 1 to 30, preferably from 1 to 20, or from 1 to 12, or from 1 to 8, or from 1 to 4. Preferably, the sum of n, m and p (as applicable in each case) is at least about 12, preferably at least about 16, more preferably at least about 20 and in some embodiments, at least about 24 or at least about 30. In each of the embodiments, the alkyl moieties (e.g., —(—C—)—) as shown can be substituted or unsubstituted alkyl moieties. In these embodiments, V can be a multifunctional bridging moiety as generally and specifically described herein. V can be a moiety independently selected from the group consisting of N, O, S, disulfide, carbonyl, ester, amide, urethane, urea, hydrazine, alkene, and alkyne.

In another (third) general embodiment, the substituted organic compound can comprise three or more independently selected multi-ring structures, Z₁, Z₂, Z₃ . . . Zₙ each joined by a linking moiety, L. In one embodiment, for example, the multivalent compound of the invention can be a trimer comprising three or more independently selected multi-ring structures, Z₁, Z₂, Z₃, each bonded to a linking moiety, L, the where L can be a linking moiety represented by the formula (D-V) 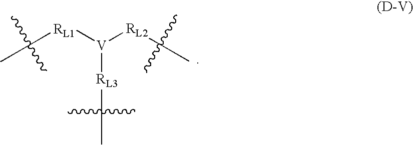
Here, the multi-ring structures Z₁, Z₂, Z₃ can be covalently bonded to the linking moiety. The multi-ring structures, Z₁, Z₂, Z₃ can each be indole or indole-related compounds (e.g., the multivalent phospholipase inhibitor) as described herein above, and as further detailed hereinafter. R_{L1}, R_{L2} and R_{L3} can each be a moiety independently selected from the group consisting of alkyl, substituted alkyl, alkenyl, substituted alkenyl, alkynyl, carbocyclic, heterocyclic, poly(ethylene oxyl), and polyester. In some embodiments, each R_{L1}, R_{L2} and R_{L3} can be an independently selected non-repeating moiety (e.g., a moiety other than an oligomer or polymer) and can be an independently selected from the group consisting of alkyl, substituted alkyl, alkenyl, substituted alkenyl, alkynyl, carbocyclic, heterocyclic. In these embodiments, V can be a multifunctional bridging moiety as generally and specifically described herein. V can be a moiety independently selected from the group consisting of N, O, S, disulfide, carbonyl, ester, amide, urethane, urea, hydrazine, alkene, and alkyne.

For example, in some preferred embodiments, the linking moiety, L, can be a linking moiety represented by the formula selected from (D-V-A) 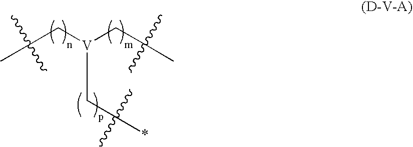
with n, m and p being independently selected non-zero integers. The integers n, m and p can each be independently selected as ranging from 1 to 50, preferably from 1 to 30, preferably from 1 to 20, or from 1 to 12, or from 1 to 8, or from 1 to 4. Preferably, the sum of any two of integers n, m and p (e.g., (n+m) or (n+p) or (m+p)) is at least about 12, preferably at least about 16, more preferably at least about 20 and in some embodiments, at least about 24 or at least about 30. In each of the embodiments, the alkyl moieties (e.g., —(—C—)—) as shown can be substituted or unsubstituted alkyl moieties. In these embodiments, V can be a multifunctional bridging moiety as generally and specifically described herein. V can be a moiety independently selected from the group consisting of N, O, S, disulfide, carbonyl, ester, amide, urethane, urea, hydrazine, alkene, and alkyne.

In general (for all embodiments), the total atomic distance between the multi-ring structures Z (e.g., including the multifunctional bridge moiety and/or any linking moieties, L) can be a length of at least twenty atoms in the shortest chain through which at least two of the two or more multi-ring structures, Z, are joined, and in some embodiments in each case, through which each of the two or more multi-ring structures, Z, are joined. Atomic distances for (e.g., carbocyclic or heterocyclic) ring structures is considered to be based on the nearest approximate number of C—C bond lengths in a straight line path across the (e.g., carbocyclic or heterocyclic) ring structures. In some embodiments, the total atomic distance between the multi-ring structures Z (e.g., including the multifunctional bridge moiety and/or any linking moieties, L) can be a length ranging from about 20 to about 500 atoms, preferably from about 20 to about 400 atoms, or from about 20 to about 300 atoms, or from about 20 to about 200 atoms, or from about 20 to about 100 atoms, or from about 20 to about 50 atoms, or from about 20 to about 40 atoms, or from about 20 to about 30 atoms, in each case, in the shortest chain through which at least two of the two or more multi-ring structures, Z, are joined, and in some embodiments in each case, through which each of the two or more multi-ring structures, Z, are joined.

Preferred compounds of the first aspect of the invention, suitable as multivalent phospholipase inhibitors, can be a compound represented by a formula selected from 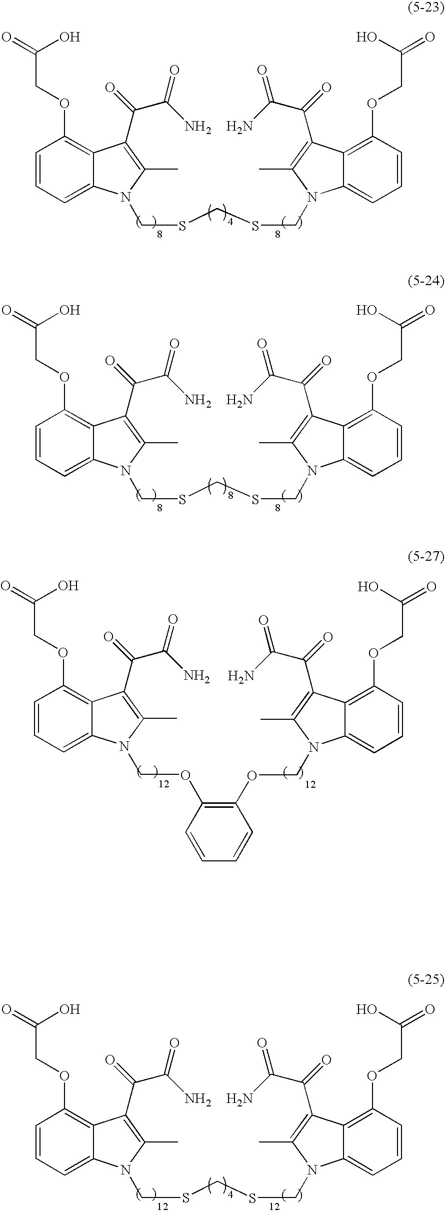 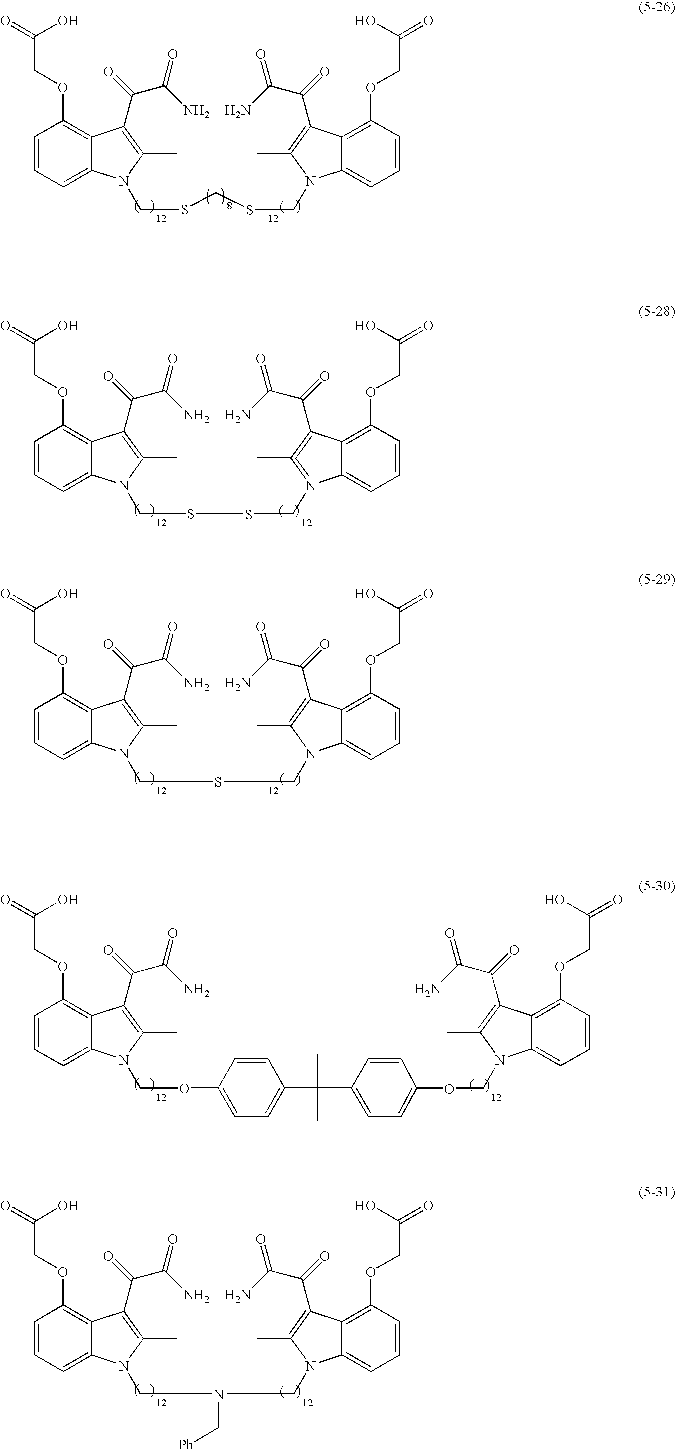 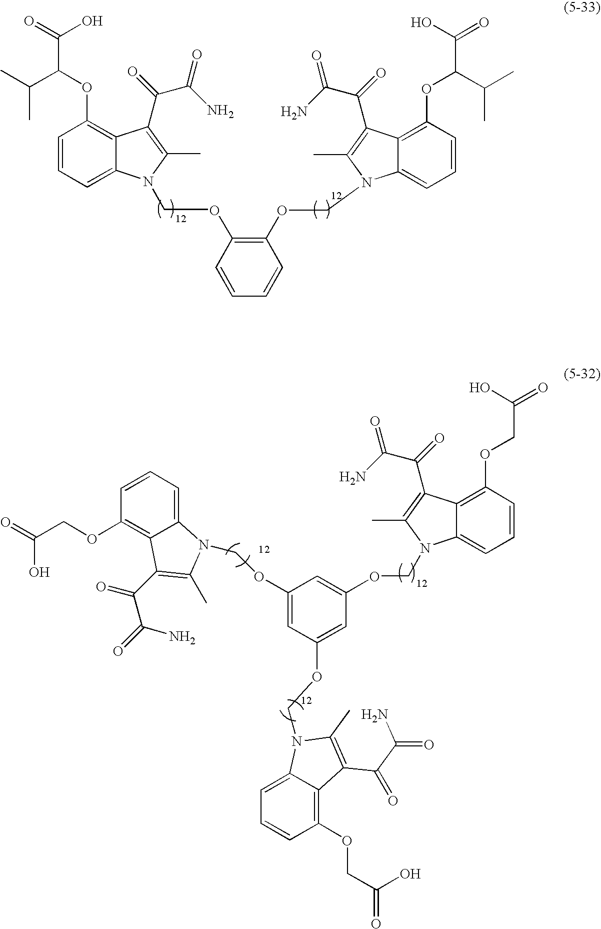
Other Compounds, Suitable as Phospholipase Inhibitors or Moieties

Composition of matter within the second aspect of the invention can comprise a substituted organic compound or a salt thereof, where the substituted organic compound is represented by a formula selected from among the following.

Especially preferred moieties having phospholipase inhibiting activity can be selected, for example, from moieties having C-4 acidic groups, such as

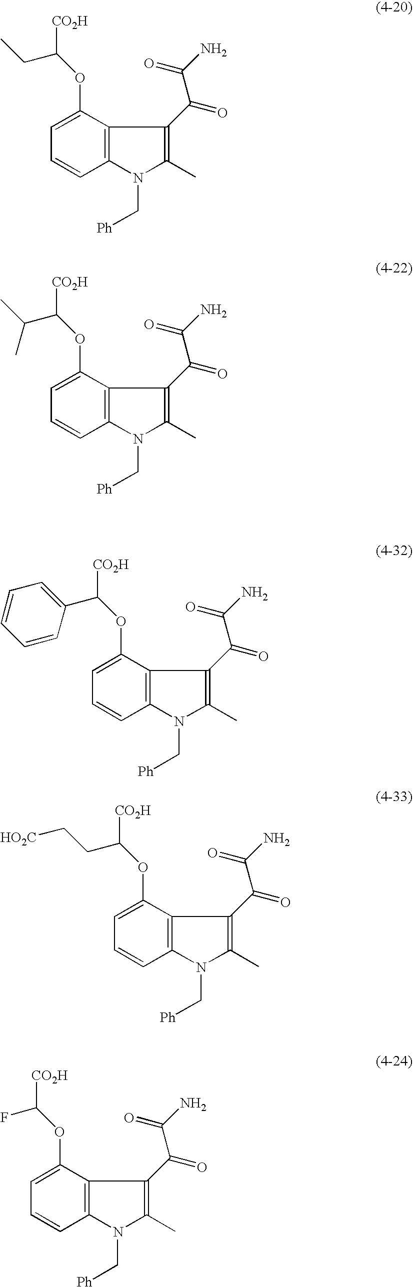

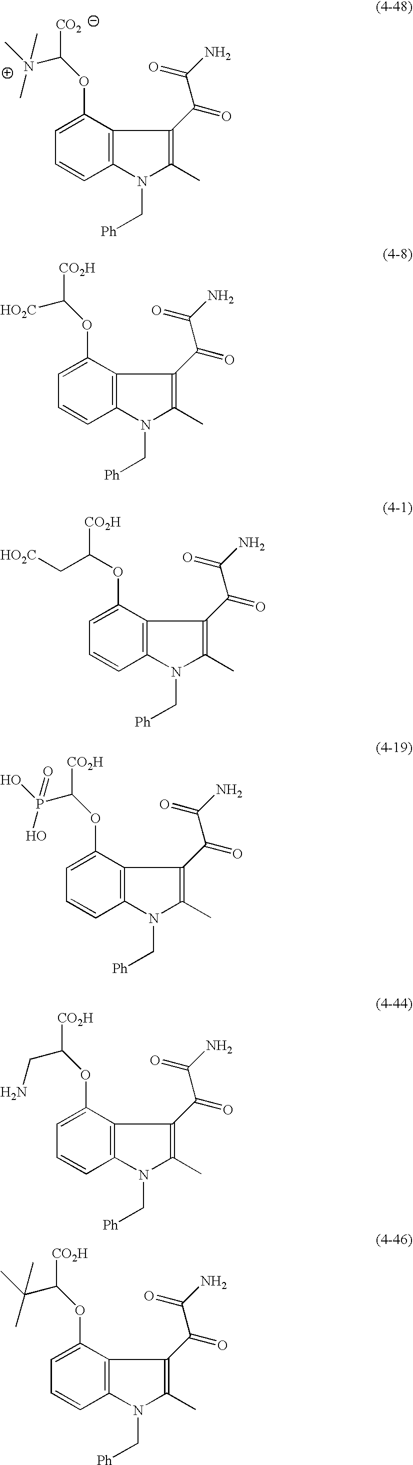

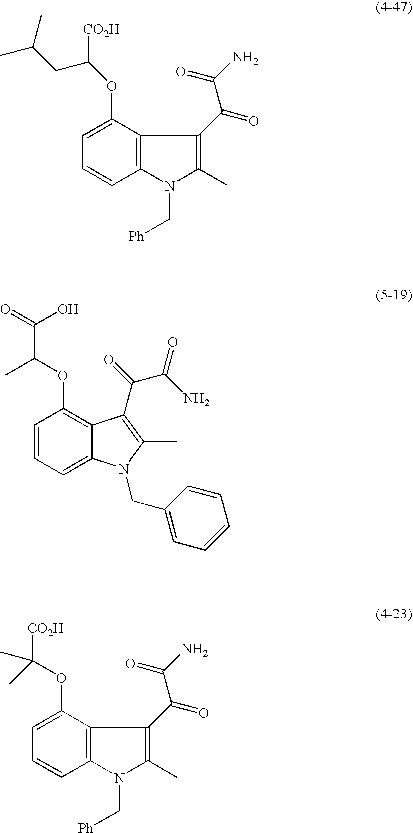

Especially preferred moieties having phospholipase inhibiting activity can also be selected, for example, from moieties having C-4 amide groups, such as

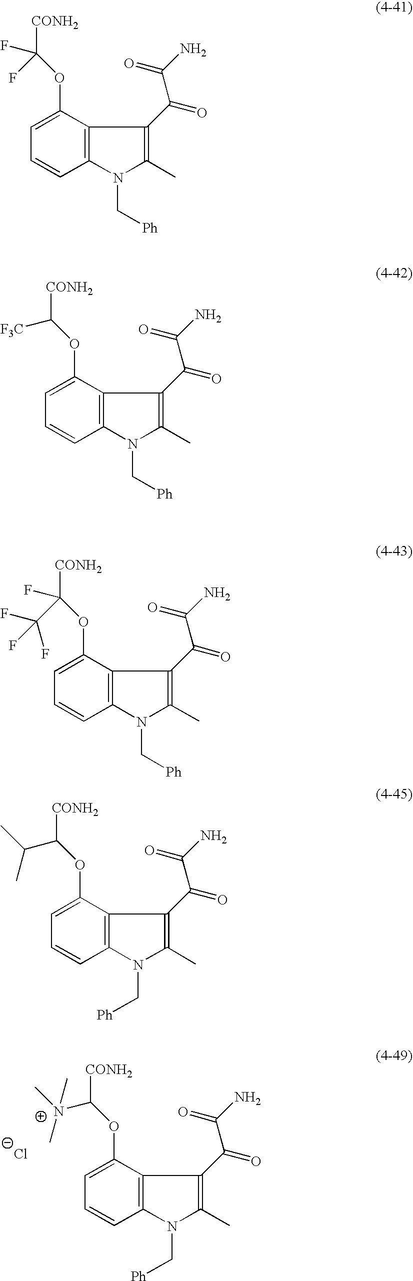

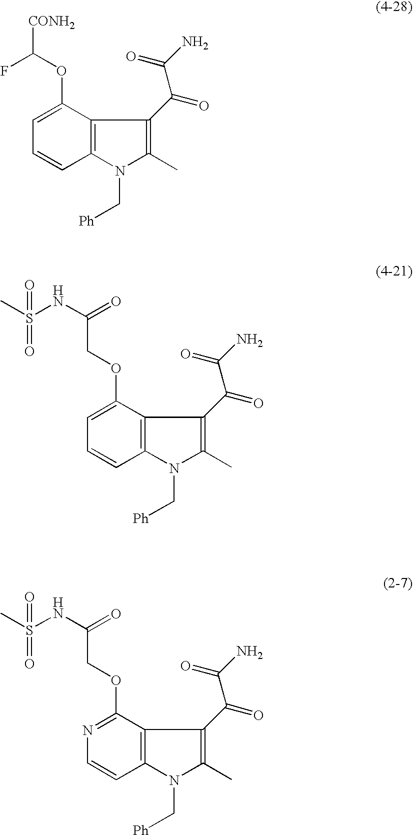

Especially preferred moieties having phospholipase inhibiting activity can also be selected, for example, from moieties having azaindole and azaindole related multi-ring structures, such as

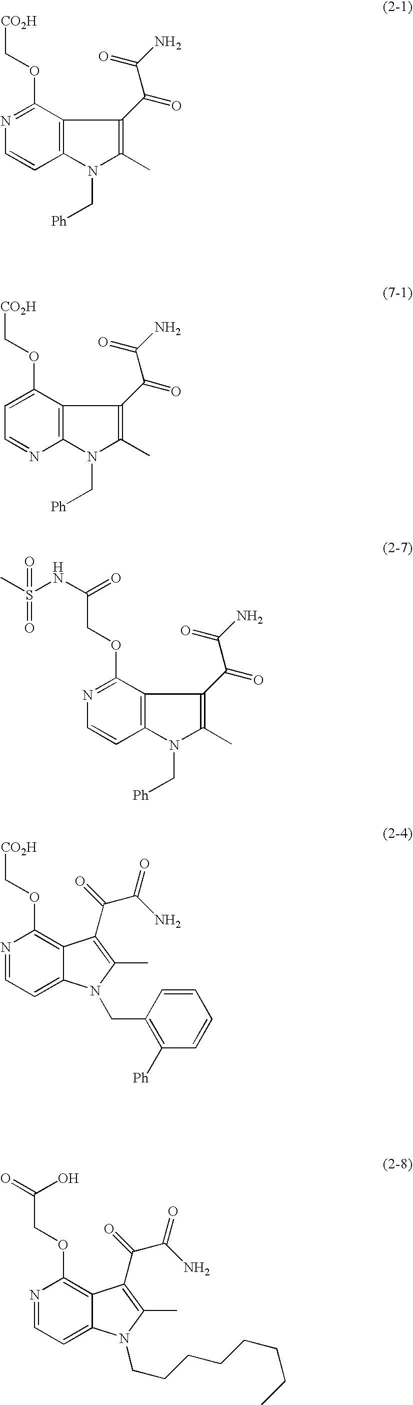

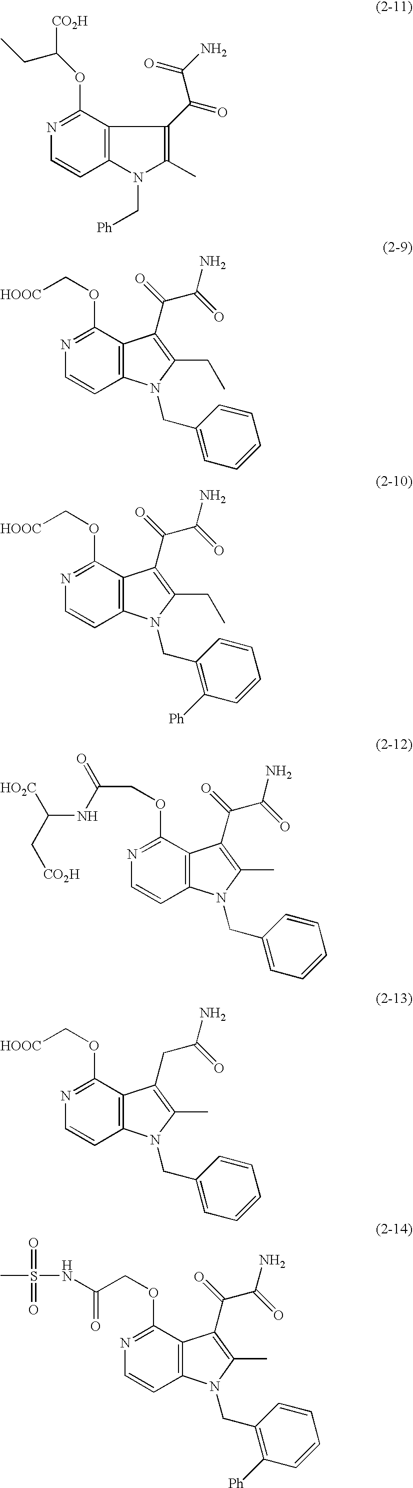

Other moieties having phospholipase inhibiting activity can also be selected, for example, including moieties such as

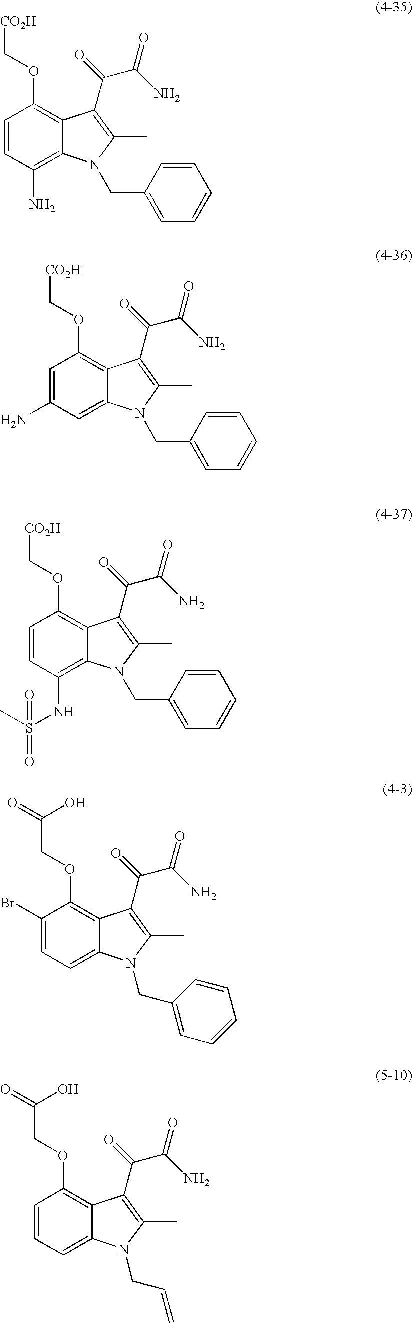

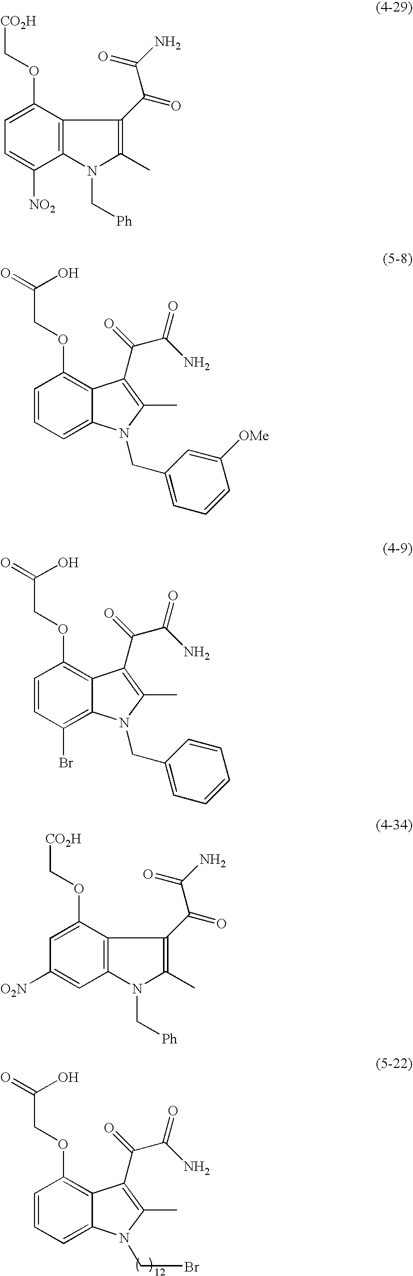

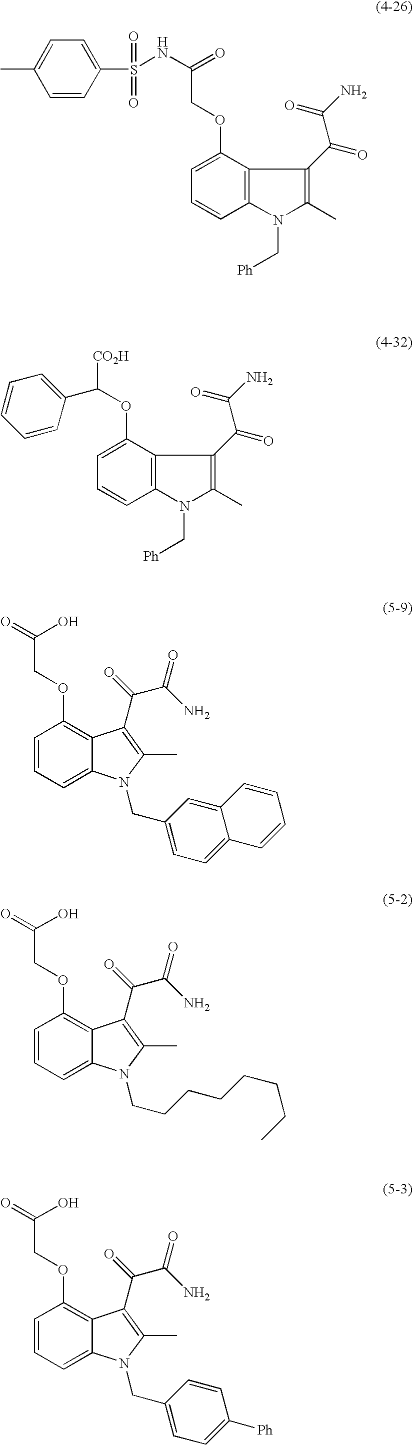

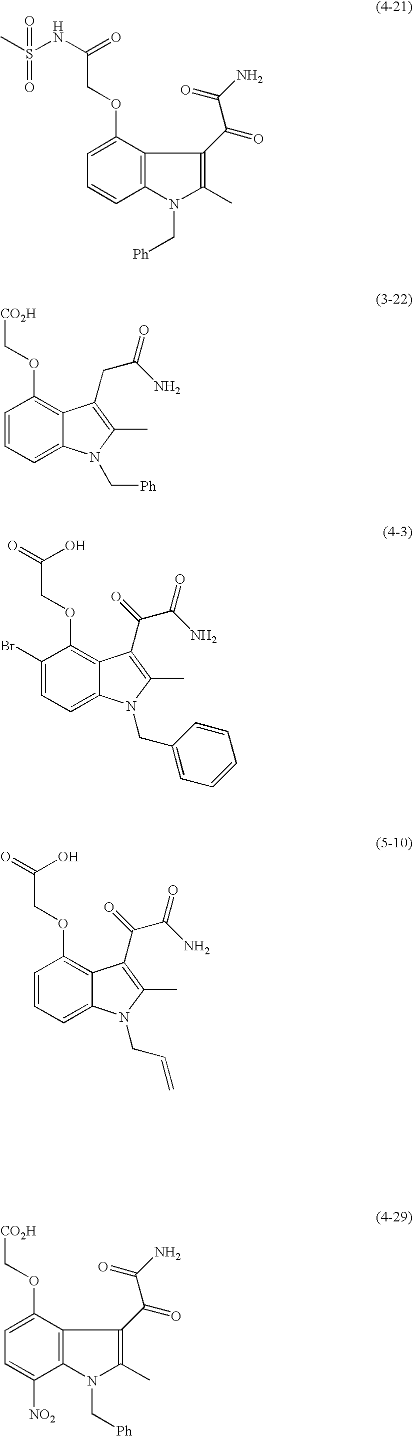

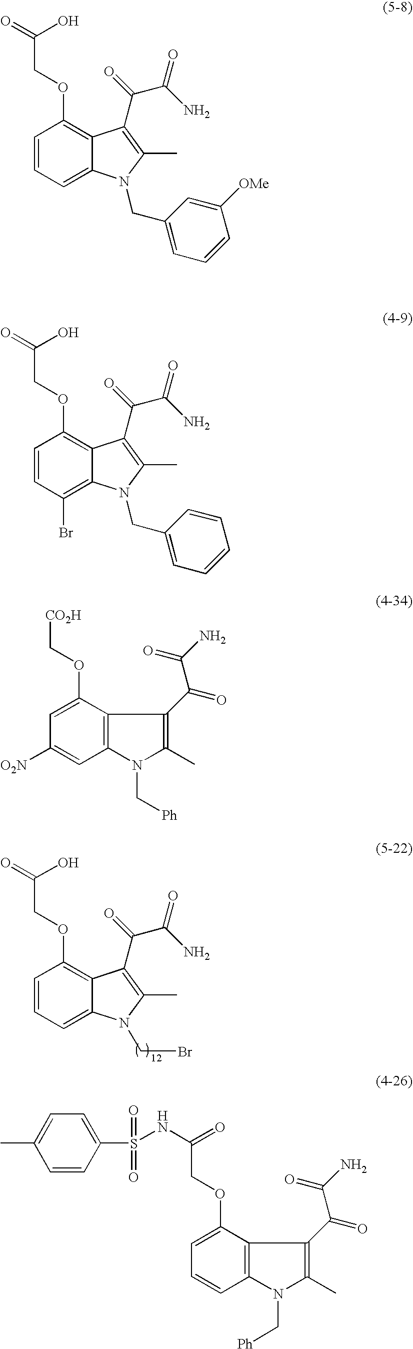

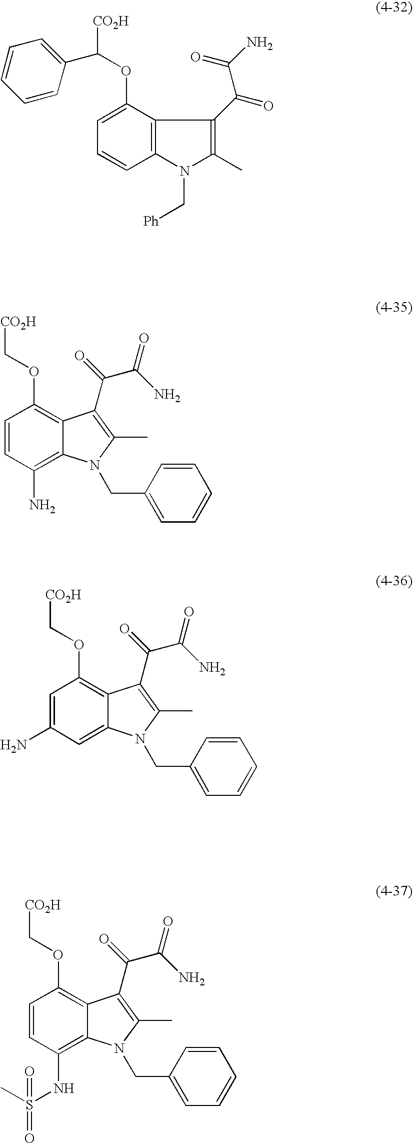

In particular, these compounds as well as other compounds can be suitably employed as phospholipase inhibiting compounds.

Localization within the Gastrointestinal Lumen Via Non-Absorbtion

In preferred approaches, the phosphate inhibitor can be an inhibitor that is substantially not absorbed from the gastrointestinal lumen into gastrointestinal mucosal cells. As such, “not absorbed” as used herein can refer to inhibitors adapted such that a significant amount, preferably a statistically significant amount, more preferably essentially all of the phospholipase inhibitor, remains in the gastrointestinal lumen. For example, at least about 80% of phospholipase inhibitor remains in the gastrointestinal lumen, at least about 85% of phospholipase inhibitor remains in the gastrointestinal lumen, at least about 90% of phospholipase inhibitor remains in the gastrointestinal lumen, at least about 95%, at least about 98%, preferably at least about 99%, and more preferably at least about 99.5% remains in the gastrointestinal lumen (in each case based on a statistically relevant data set). Reciprocally, stated in terms of serum bioavailability, a physiologically insignificant amount of the phospholipase inhibitor is absorbed into the blood serum of the subject following administration to a subject. For example, upon administration of the phospholipase inhibitor to a subject, not more than about 20% of the administered amount of phospholipase inhibitor is in the serum of the subject (e.g., based on detectable serum bioavailability following administration), preferably not more than about 15% of phospholipase inhibitor, and most preferably not more than about 10% of phospholipase inhibitor is in the serum of the subject. In some embodiments, not more than about 5%, not more than about 2%, preferably not more than about 1%, and more preferably not more than about 0.5% is in the serum of the subject (in each case based on a statistically relevant data set). In some cases, localization to the gastrointestinal lumen can refer to reducing net movement across a gastrointestinal mucosa, for example, by way of both transcellular and paracellular transport, as well as by active and/or passive transport. The phospholipase inhibitor in such embodiments is hindered from net permeation of a gastrointestinal mucosal cell in transcellular transport, for example, through an apical cell of the small intestine; the phospholipase inhibitor in these embodiments is also hindered from net permeation through the “tight junctions” in paracellular transport between gastrointestinal mucosal cells lining the lumen. The term “not absorbed” is used interchangeably herein with the terms “non-absorbed,” “non-absorbedness,” “non-absorption” and its other grammatical variations.

In some embodiments, detailed further below, an inhibitor or inhibiting moiety can be adapted to be non-absorbed by modifying the charge and/or size, particularly, as well as additionally other physical or chemical parameters of the phospholipase inhibitor. For example, in some embodiments, the phospholipase inhibitor is constructed to have a molecular structure that minimizes or nullifies absorption through a gastrointestinal mucosa. The absorption character of a drug can be selected by applying principles of pharmacodynamics, for example, by applying Lipinsky's rule, also known as “the rule of five.” As a set of guidelines, Lipinsky shows that small molecule drugs with (i) molecular weight, (ii) number of hydrogen bond donors, (iii) number of hydrogen bond acceptors, and (iv) water/octanol partition coefficient (Moriguchi logP) each greater than a certain threshold value generally do not show significant systemic concentration. See Lipinsky et al, Advanced Drug Delivery Reviews, 46, 2001 3-26, incorporated herein by reference. Accordingly, non-absorbed phospholipase inhibitors can be constructed to have molecule structures exceeding one or more of Lipinsky's threshold values, and preferably two or more, or three or more, or four or more or each of Lipinsky's threshold values. See also Lipinski et al., Experimental and computational approaches to estimate solubility and permeability in drug discovery and development settings, Adv. Drug Delivery Reviews, 46:3-26 (2001); and Lipinski, Drug-like properties and the causes o poor solubility and poor permeability, J. Pharm. & Toxicol. Methods, 44:235-249 (2000), incorporated herein by reference. In some preferred embodiments, for example, a phospholipase inhibitor of the present invention can be constructed to feature one or more of the following characteristics: (i) having a MW greater than about 500 Da; (ii) having a total number of NH and/or OH and/or other potential hydrogen bond donors greater than about 5; (iii) having a total number of O atoms and/or N atoms and/or other potential hydrogen bond acceptors greater than about 10; and/or (iv) having a Moriguchi partition coefficient greater than about 10⁵, i.e., logP greater than about 5. Any art known phospholipase inhibitors and/or any phospholipase inhibiting moieties described below can be used in constructing a non-absorbed molecular structure.

Preferably, the permeability properties of the compounds are screened experimentally: permeability coefficient can be determined by methods known to those of skill in the art, including for example by Caco-2 cell permeability assay. The human colon adenocarcinoma cell line, Caco-2, can be used to model intestinal drug absorption and to rank compounds based on their permeability. It has been shown, for example, that the apparent permeability values measured in Caco-2 monolayers in the range of 1×10⁻⁷ cm/sec or less typically correlate with poor human absorption (Artursson P, K. J. (1991). Permeability can also be determined using an artificial membrane as a model of a gastrointestinal mucosa. For example, a synthetic membrane can be impregnated with e.g. lecithin and/or dodecane to mimic the net permeability characteristics of a gastrointestinal mucosa. The membrane can be used to separate a compartment containing the phospholipase inhibitor from a compartment where the rate of permeation will be monitored. “Correlation between oral drug absorption in humans and apparent drug.” Biochemical and Biophysical Research Communications 175(3): 880-885.) Also, parallel artificial membrane permeability assays (PAMPA) can be performed. Such in vitro measurements can reasonably indicate actual permeability in vivo. See, for example, Wohnsland et al. J. Med. Chem., 2001, 44:923-930; Schmidt et al., Millipore corp. Application note, 2002, no AN1725EN00, and no AN1728EN00, incorporated herein by reference. The permeability coefficient is reported as its decimal logarithm, Log Pe.

In some embodiments, the phospholipase inhibitor permeability coefficient Log Pe is preferably lower than about −4, or lower than about −4.5, or lower than about −5, more preferably lower than about −5.5, and even more preferably lower than about −6 when measured in the permeability experiment described in Wohnsland et al. J. Med. Chem. 2001, 44. 923-930.

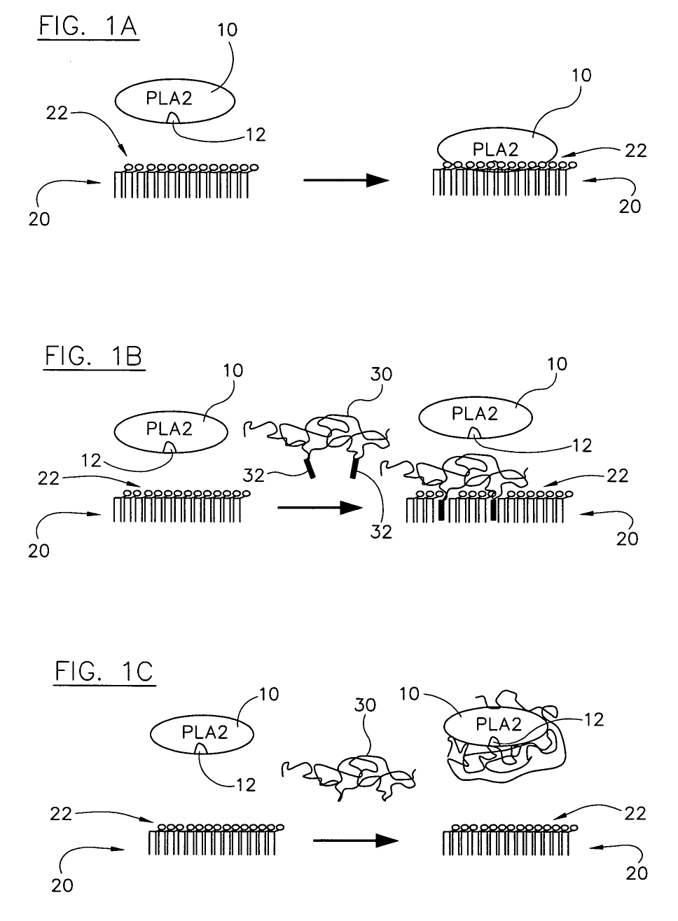

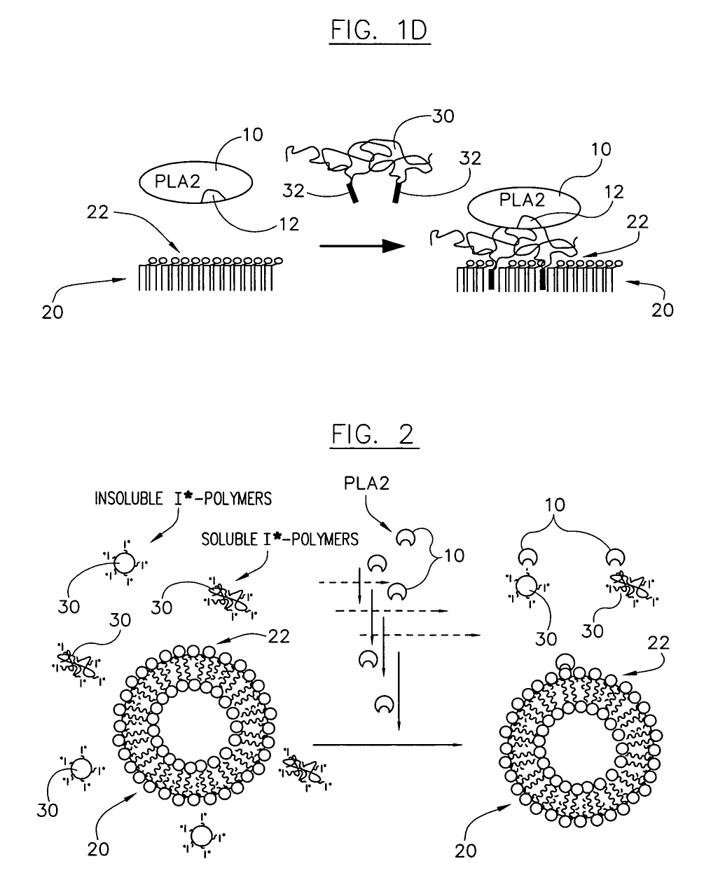

As noted, in one general embodiment, the phospholipase inhibitor can comprise or consist essentially of an oligomer or a polymer. Generally, such polymer inhibitor can be sized to be non-absorbed, and can be adapted to be enzyme-inhibiting, for example based on one or more or a combination of features, such as charge characteristics, relative balance and/or distribution of hydrophilic/hydrophobic character, and molecular structure. The oligomer or polymer in this general embodiment is preferably soluble, and can preferably be a copolymer (including polymers having two monomer-repeat-units, terpolymers and higher-order polymers), including for example random copolymer or block copolymer. The oligomer or polymer can generally include one or more ionic monomer moieties such as one or more anionic monomer moieties. The oligomer or polymer can generally include one or more hydrophobic monomer moieties. The oligomer or polymer inhibitor can interact with the phospholipase, for example with a specific site thereon, preferably with the catalytic site bearing face (e.g., the i-face) of a phospholipase such as phospholipid-A₂. As described below in connection with FIGS. 1A through 1D, the oligomer or polymer can hinder access of a phospholipase to a phospholipids, for example by interacting with the phospholipase, or by interacting with the phospholipid substrate, or by interacting with both the phospholipase and the phospholipid. As described below in connection with FIG. 1C, the inhibitor can be effective for scavenging phospholipase, for example, within a fluid such as an aqueous phase of the gastrointestinal tract.

Specific polymers and specific monomers for such oligomer or polymer inhibitor can be those included in the following discussion, in connection with the general embodiment in which an oligomer or polymer moiety is covalently linked to a phospholipase inhibiting moiety.

In a second general embodiment, a phospholipase inhibitor can comprises a phospholipase inhibiting moiety linked, coupled or otherwise attached to a multifunctional bridge moiety, such as an oligomer moiety or a polymer moiety or a non-repeating multifunctional bridge moiety, where such oligomer moiety or polymer moiety or non-repeating moiety can be a hydrophobic moiety, hydrophilic moiety, and/or charged moiety. In some preferred embodiments (where a larger number of phospholipase inhibiting moieties are to be presented), the phospholipase inhibiting moiety is coupled to a polymer moiety. In some embodiments (where a relatively smaller number of phospholipase inhibiting moieties are to be presented), the phospholipase inhibiting moiety is coupled to an oligomer moiety, or non-repeating multifunctional bridge moiety as described above.

In one more specific approach within this general embodiment, the polymer moiety may be of relatively high molecular weight, for example ranging from about 1000 Da to about 500,000 Da, preferably in the range of about 5000 to about 200,000 Da, and more preferably sufficiently high to hinder or preclude (net) absorption through a gastrointestinal mucosa. Large polymer moieties may be advantageous, for example, in scavenging approaches involving relatively large, soluble or insoluble (e.g., cross-linked) polymers having multiple inhibiting moieties (e.g., as discussed below in connection with FIG. 2).

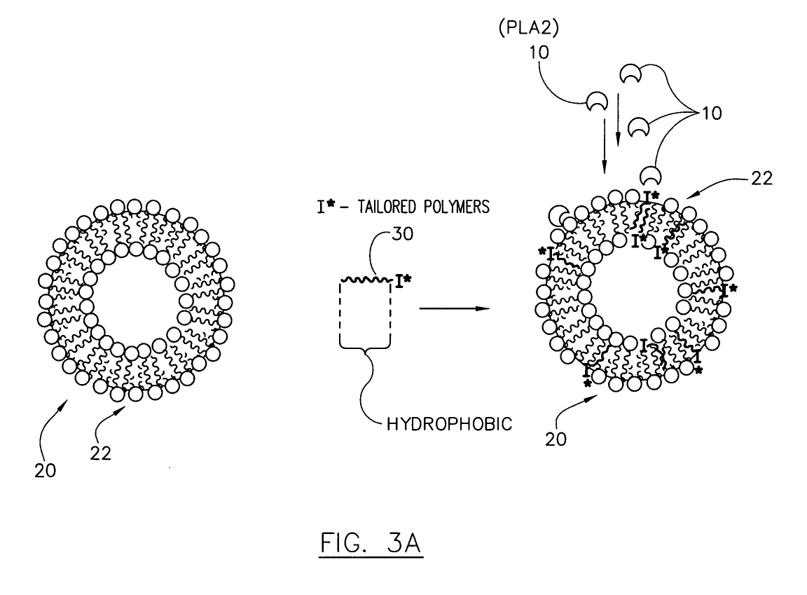

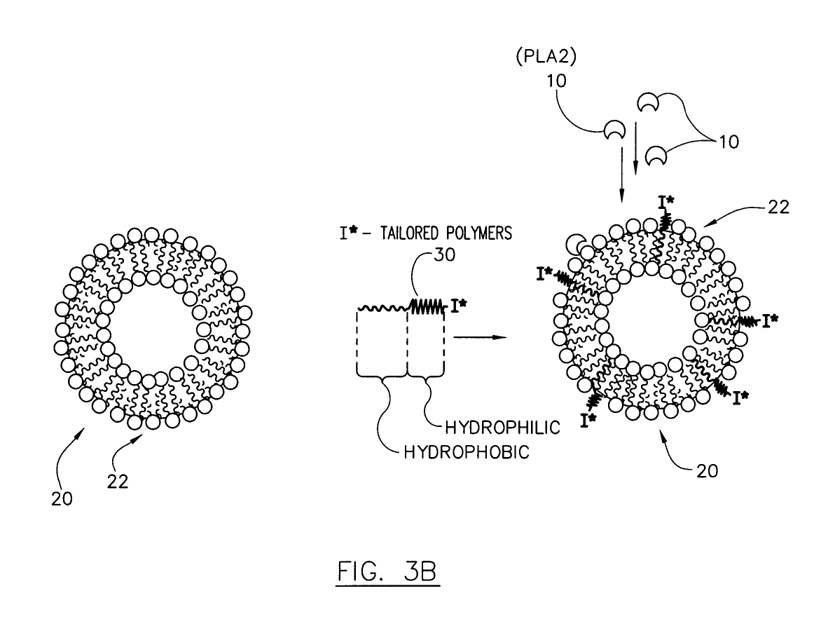

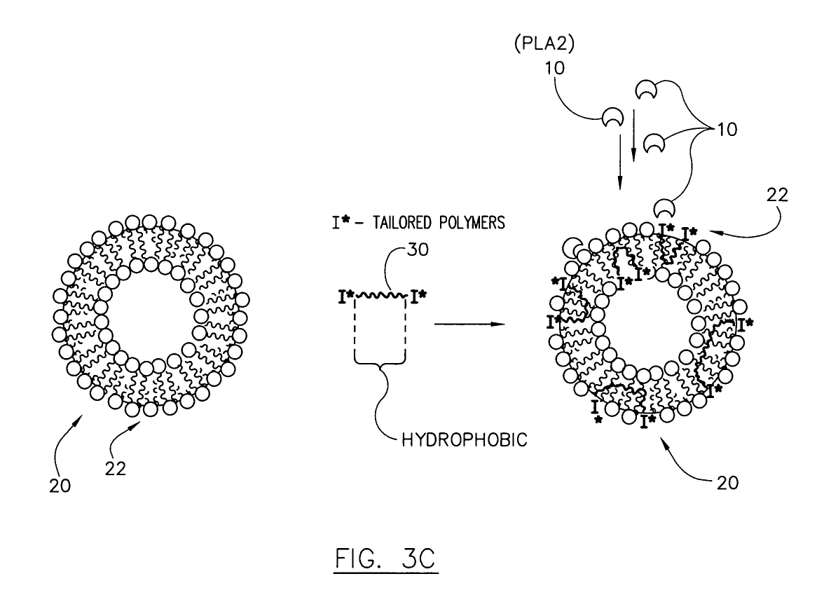

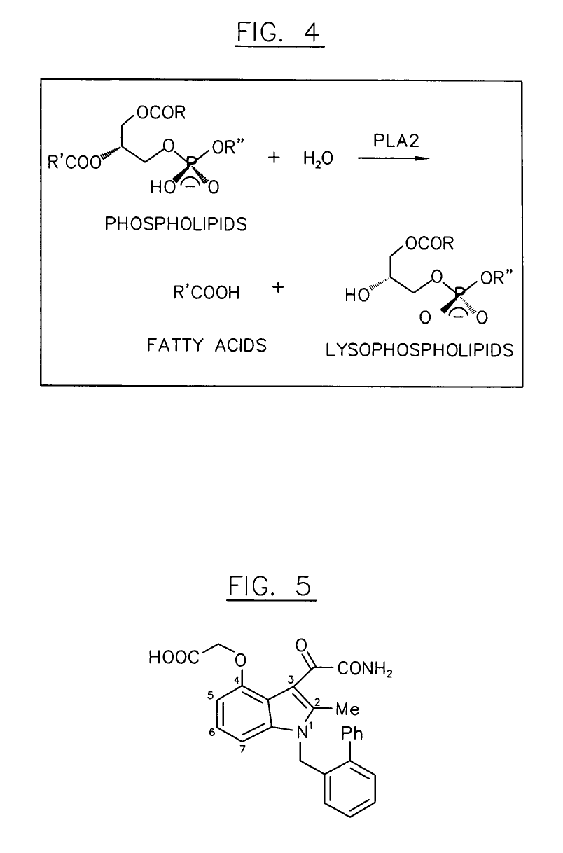

In an alternative more specific approach within this general embodiment, the oligomer or polymer moiety may be of low molecular weight, for example not more than about 5000 Da, and preferably not more than about 3000 Da and in some cases not more than about 1000 Da. Preferably within this approach, the oligomer or polymer moiety can consist essentially of or can comprise a block of hydrophobic polymer, allowing the inhibitor to associate with a water-lipid interface (e.g., of a lipid aggregate as described below in connection with FIGS. 3A through 3C).

In any case, and particularly for each of the immediately aforementioned more specific approaches for this general embodiment, a phospholipase inhibiting moiety may be linked to at least one repeat unit of a polymer moiety. Hence, the phospholipase inhibitor can comprise a repeat unit, an oligomer or a polymer according to the following formula (A): 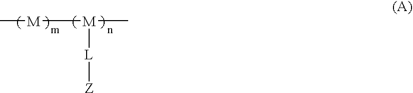
where n and m are each integers (at least one of which is a non-zero integer), M represents a monomer moiety, L is an optional linking moiety, (e.g., a chemical linker), and Z is a phospholipase inhibiting moiety, preferably a PL A₂ inhibiting moiety, and most preferably a PL A₂ 1B inhibiting moiety. In some embodiments, the integer m is zero. Generally, n can be less than 1000; in some embodiments, n can be less than about 500. The integer n can range from 1 to 500, from 1 to 400, from 1 to 300, from 1 to 200, from 1 to 100, from 1 to 50, from 1 to 20 or from 1 to 10. Preferably, n is at least 2 and less than about 500. The integer, n, can range from 2 to about 400, preferably from 2 to about 300, from 2 to about 200, and more preferably from 2 to about 100, from 2 to about 50, or from 2 to about 35, and from 2 to about 20, or from 2 to about 10 or from 3 to about 10. In some particular embodiments, the number of phospholipase inhibiting moieties can be lower, with the integer n ranging from 2 to about 8, or from 3 to about 8. In some other embodiments, the number of phospholipase inhibiting moieties is still lower, with n ranging from 2 to about 6, or from 3 to about 6. In certain embodiments, the integer n can range from 2 to 4, or from 3 to 4.

Generally, M represents one or more monomer moiety. Accordingly, each M can independently include one or more of a first monomer moiety, M₁, a second monomer moiety, M₂, a third monomer moiety, M₃, a fourth monomer moiety, M₄, a fifth monomer moiety, M₅, a sixth monomer moiety, M₆, etc., in each case with M₁ through M₆ being different from each other.

In one approach, each M can be one monomer moiety (the same type repeat unit), such that the phospholipase inhibitor can comprises a repeat unit, an oligomer or a polymer having the formula (A-1) 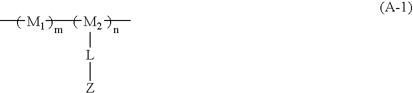
wherein m is a non-zero integer, n is a non-zero integer, M₁ is a first monomer moiety, M₂ is a second monomer moiety, the second monomer moiety being the same as or different than the first monomer moiety, L is an optional linking moiety and Z is a phospholipase inhibiting moiety. In this case, each of M₁ and M₂ can be the same, whereby the phospholipase inhibitor comprises a homopolymer repeat unit, oligomer or polymer moiety. Alternatively, M₁ and M₂ can be different, whereby the phospholipase inhibitor comprises a copolymer repeat unit, oligomer or polymer moiety. The copolymer repeat unit, oligomer or polymer moiety can be a random copolymer or a block copolymer repeat unit, oligomer or polymer moiety. Generally, in some embodiments, n can be less than about 500. Preferably, n is at least 2 and less than about 500. (Preferred n can be as described above in connection with formula A).

In a preferred embodiment, the phospholipase inhibitor can comprises an oligomer or polymer moiety having a first repeat unit and a second repeat unit, the first repeat unit having a formula (A-1), above, wherein n is one and m is one or more, whereby the oligomer or polymer moiety of the phospholipase inhibitor is a random copolymer comprising the first and second repeat units. Preferably, m ranges from four to fifty and n is two. More preferably, m is at least four and n is one. The second repeat unit can be of any suitable monomer type.

In some preferred embodiments, for example, where the oligomer or polymer moiety is of a relatively low molecular weight, the oligomer or polymer moiety can be a tailored oligomer or polymer moiety adapted to associate with a water-lipid interface (e.g., of a lipid aggregate as described below in connection with FIGS. 3A through 3C). In such embodiments, the oligomer or polymer moiety can consist essentially of or can comprise a region or block having a relatively hydrophobic character, allowing for integral association with the lipid aggregate (e.g., micelle or vesicle).

For example, in this regard, the phospholipase inhibitor can comprises a compound of the formula (B) 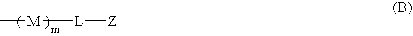
wherein m is a non-zero integer, M is a monomer moiety, L is an optional linking moiety and Z is a phospholipase inhibiting moiety. Such oligomer or polymer moieties having a single covalently-linked inhibiting moiety can be referred to herein as a “singlet” inhibitor (or a monovalent inhibitor) and can be effective, for example, as illustrated and discussed below in connection with FIGS. 3A and 3B.

As another example, the phospholipase inhibitor can comprise an oligomer or polymer moiety covalently linked to a phospholipase inhibiting moiety, the phospholipase inhibitor comprising a compound having the formula (C) 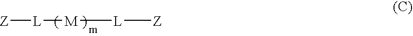
wherein m is a non-zero integer, M is a monomer moiety, L are each independently selected optional linking moieties and Z are each, independently selected phospholipase inhibiting moieties. As a further example, the phospholipase inhibitor can comprise an oligomer or polymer moiety covalently linked to a phospholipase inhibiting moiety, the phospholipase inhibitor comprising a compound having the formula (C-1) 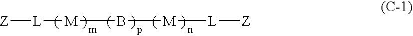
wherein m is a non-zero integer, n is a non-zero integer, p is a non-zero integer, M are each independently selected monomer moieties, B is a bridging moiety, L are each independently selected optional linking moieties, and Z are each independently selected phospholipase inhibiting moieties. In each of these two cases, such oligomer or polymer moieties having two covalently-linked inhibiting moieties can be referred to herein as a “dimer” inhibitor and can be effective, for example, as illustrated and discussed below in connection with Formula C.

In these immediately preceding singlet and dimer embodiments, M represents one or more monomer moiety, and each M can independently include one or more of a first monomer moiety, M₁, a second monomer moiety, M₂, a third monomer moiety, M₃, a fourth monomer moiety, M₄, a fifth monomer moiety, M₅, a sixth monomer moiety, M₆, etc., in each case with M₁ through M₆ being different from each other. In some cases, M can generally comprise at least a first monomer moiety, M₁, and optionally further comprises in combination therewith a second monomer moiety, M₂, different from the first monomer moiety. M can consist essentially of a first monomer, M₁, whereby the phospholipase inhibitor comprises a homopolymer oligomer or polymer moiety or moieties. Alternatively, M can comprise a first monomer, M₁, and a second monomer, M₂ different from the first monomer, whereby the phospholipase inhibitor comprises a copolymer oligomer or polymer moiety or moieties. The copolymer oligomer or polymer moiety can be random copolymer or a block copolymer moiety or moieties. M can generally comprise a hydrophobic monomer moiety, and can also include generally an anionic monomer moiety. In one specific example, M can comprise a first block consisting essentially of a hydrophobic first monomer, M₁, and a second block consisting essentially of a hydrophilic second monomer, M₂, with the second block being proximal to the phospholipase inhibiting moiety or moieties. In these embodiments (e.g., of formulas B, C and C-1), m can range from two to about 200, preferably from four to about fifty. In embodiment C-1, n can likewise range from two to about 200, preferably from four to about fifty, and p can range from 1 to 20, preferably 1 to 10, and in some cases 1 to 4.

Hence, in one embodiment, the phospholipase inhibitor can comprise a compound of the formula (C-2) 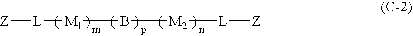
wherein m is a non-zero integer, n is a non-zero integer, p is a non-zero integer, M₁ is a first monomer moiety, M₂ is a second monomer moiety, the second monomer moiety being the same as or different than the first monomer moiety, B is a bridging moiety, L are each independently selected optional linking moieties, and Z are each independently selected phospholipase inhibiting moieties. In this embodiment, m and n can each be independently selected integers ranging from two to about 500, or from four to about 500, preferably ranging from four to about 100, and most preferably ranging from four to fifty.
Linking Moiety

The linking moiety L, in each of the described embodiments (including embodiments in which a phospholipase inhibiting moiety is linked to a multifunctional bridge such as a polymer moiety, an oligomer moiety, or a non-repeating moiety) can be a chemical linker, such as a bond or a other moiety, for example, comprising about 1 to about 10 atoms that can be hydrophilic and/or hydrophobic. In some embodiments, the linker can be longer, including for example where the linking moiety is also the bridge moiety, comprising for example from 1 to about 100 atoms that can be hydrophilic and/or hydrophobic. In some embodiments, the linker moiety can range from 10 to 100 atoms along a shortest path between inhibiting moiety, in some embodiments is at least 20 atoms along such a shortest path, preferably from about 20 to about 100 or from 20 to about 50 atoms. The linking moiety links, couples, or otherwise attaches the phospholipase inhibiting moiety Z to another inhibiting moiety Z, or to a non-repeating bridge moiety, or to an oligomer moiety, or to a polymer moiety (for example to a backbone of the polymer moiety). In one embodiment, the linking moiety can be a polymer moiety grafted onto a polymer backbone, for example, using living free radical polymerization approaches known in the art.

Polymer Moieties

Generally, with respect to embodiments comprising a polymer moiety, a number of polymers can be used including, for example, synthetic and/or naturally occurring aliphatic, alicyclic, and/or aromatic polymers. In preferred embodiments, the polymer moiety is stable under physiological conditions of the gastrointestinal (GI) tract. By “stable” it is meant that the polymer moiety does not degrade or does not degrade significantly or essentially does not degrade under the physiological conditions of the GI tract. For instance, at least about 90%, preferably at least about 95%, and more preferably at least about 98%, and even more preferably at least about 99% of the polymer moiety remains un-degraded or intact after at least about 5 hours, at least about 10 hours, at least about 24 hours, or at least about 48 hours of residence in a gastrointestinal tract (in each case based on a statistically relevant data set). Stability in a gastrointestinal tract can be evaluated using gastrointestinal mimics, e.g., gastric mimics or intestinal mimics of the small intestine, which approximately model the physiological conditions at one or more locations within a GI tract.

The polymer moiety may be soluble or insoluble, existing for example as dispersed micelles or particles, such as colloidal particles or (insoluble) macroscopic beads. In some embodiments, the polymer moiety presents as insoluble porous particles. In preferred embodiments, the polymer moiety is soluble or exists as colloidal dispersions under the physiological conditions of the gastrointestinal tract, for example, at a location within the GI tract where the phospholipase inhibiting moiety acts, e.g., within the gastrointestinal lumen of the small intestine.

Polymer moieties can be hydrophobic, hydrophilic, amphiphilic, uncharged or non-ionic, negatively or positively charged, or a combination thereof, and can be organic or inorganic. Inorganic polymers, also referred to as inorganic carriers in some cases, include silica (e.g., multi-layered silica), diatomaceous earth, zeolite, calcium carbonate, talc, and the like.

The polymer architecture of the polymer moiety can be linear, grafted, comb, block, star and/or dendritic, preferably selected to produce desired solubility and/or stability characteristics as described above. The architecture may involve a macromolecular scaffold, and in some embodiments the scaffold may form particles that may be porous or non-porous. The particles may be of any shape, including spherical, elliptical, globular, or irregularly-shaped particles. Preferably the particles are composed of a crosslinked organic polymer derived from, e.g., styrenic, acrylic, methacrylic, allylic, or vinylic monomers, or produced by polycondensation such as polyester, polyamide, melamin and phenol formol condensates, or derived from semi-synthetic cellulose and cellulose-like materials, such as cross-linked dextran or agarose (e.g., Sepharose (Amersham)).

In preferred particle embodiments comprising a phospholipase inhibiting moiety linked, coupled or otherwise attached to a polymer moiety, the particles provide enough available surface area to allow binding of the phospholipase inhibiting moiety to phospholipase. For example, in order to help reduce the dose required to produce a therapeutic and/or a prophylactic benefit, the particles should exhibit specific surface area in the range of about 2 m²/gr to about 500 m²/gr, preferably about 20 m²/gr to about 200 m²/gr, more preferably about 40 m²/gr to about 100 m²/gr.

Phospholipase inhibiting moieties are preferably linked, coupled or otherwise attached to the polymer moiety on the surface of such particles and preferably at a density of about 0.05 mmol/g to about 4 mmol/g of the polymer moiety, more preferably about 0.1 mmol/g to about 2 mmol/g of the polymer moiety. The density of phospholipase inhibiting moieties can be determined, for example, taking into account the amount of overall PLA2 enzyme typically encountered in the human GI during or shortly after ingestion of a meal. PLA2 enzyme loading is reported to range from about 150-400 mg/L during the digestion phase with a total duodenal/jejunal volume ranging from about 1 to 2 liters. Based on a mole ratio of enzyme: inhibitor ranging from about 1:10 to about 1:100 (in a treatment protocol involving administering of PLA2 inhibitor during or shortly after meals), the mole content of inhibitor relative to moles polymer, expressed as immobilized inhibiting moieties within a polymer particle, can range from about 0.01 to about 100 mEq, and preferably from about 0.1 to about 50 mEq. The overall capacity of inhibiting-moiety-containing particles can be between about 0.05 to about 5 mEq/g, preferably from about 0.1 to about 2.5 mEq/g, and the oral administration of such inhibiting-moiety-containing particles can be between about 0.1 g and 10 g, and preferably between about 0.5 g to 5 g.

In the case where the polymer moiety forms porous particles, beads, or matrices, the pore dimension can be large enough to accommodate phospholipase, e.g., PL A₂, within the pores. In some embodiments, for example, porosity may be selected such that the minimum pore size is at least about 2 nm, preferably at least about 5 nm, and more preferably at least about 20 nm. Such materials can be produced by direct or inverse suspension polymerization using process additives such as diluent, porogen, and/or suspension aids, which can control size and porosity.

Polymer moieties useful in constructing non-absorbed inhibitors of the present invention can also be produced by free radical polymerization, condensation, addition polymerization, ring-opening polymerization, and/or can be derived from naturally occurring polymers, such as saccharide polymers. Further, in some embodiments, any of these polymer moieties may be functionalized.

Examples of polysaccharides useful in the present invention include materials from vegetal or animal origin, including cellulose materials, hemicellulose, alkyl cellulose, hydroxyalkyl cellulose, carboxymethylcellulose, sulfoethylcellulose, starch, xylan, amylopectine, chondroitin, hyarulonate, heparin, guar, xanthan, mannan, galactomannan, chitin, and/or chitosan. As noted above, more preferred are polymer moieties that do not degrade or that do not degrade significantly or essentially do not degrade under the physiological conditions of the GI tract, such as carboxymethylcellulose, chitosan, and sulfoethylcellulose.

When free radical polymerization is used, the polymer moiety can be prepared from various classes of monomers including, for example, acrylic, methacrylic, styrenic, vinylique dienic, whose typical examples are given thereafter: styrene, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, and combinations thereof. Functionalized versions of these monomers may also be used and any of these monomers may be used with other monomers as comonomers. For example, specific monomers or comonomers that may be used in this invention include methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, α-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, 4-acryloylmorpholine, vinyl benzoic acid (all isomers), diethylaminostyrene (all isomers), a-methylvinyl benzoic acid (all isomers), diethylamino α-methylstyrene (all isomers), p-vinylbenzene sulfonic acid, p-vinylbenzene sulfonic sodium salt, alkoxy and alkyl silane functional monomers, maleic anhydride, N-phenylmaleimide, N-butylmaleimide, butadiene, isoprene, chloroprene, ethylene, vinyl acetate, vinylformamide, allylamine, vinylpyridines (all isomers), fluorinated acrylate, methacrylates, and combinations thereof. Main chain heteroatom polymer moieties can also be used, including polyethyleneimine and polyethers such as polyethylene oxide and polypropylene oxide, as well as copolymers thereof.

Generally, the number of phospholipase inhibiting moieties Z appended to the polymer moiety can vary from about 1 to about 2000, most preferably from about 1 to about 500. These phospholipase inhibiting moieties can be arranged regularly or randomly along a backbone of the polymer moiety or can be localized in one particular region of the polymer moiety. For instance, (M) and (M-L-Z) repeat units can be arranged regularly, e.g., in sequences, or randomly along a backbone of the polymer moiety. If block copolymers are used, the phospholipase inhibiting moieties can be present on one block while not on another block.

Phospholipase Inhibiting Moieties. Generally, the phospholipase inhibiting moiety Z may be any art-known phospholipase inhibitor, and/or any phospholipase inhibiting moiety described herein. Preferably, the phospholipase inhibitor comprises a phospholipase inhibiting moiety that is active under the physiological conditions of the GI tract, e.g. within the pH range prevailing within the gastrointestinal lumen, i.e., from about 5 to about 8, and preferably under physiological conditions prevailing at a location within the GI tract where the phospholipase inhibiting moiety acts, e.g., within the gastrointestinal lumen of the small intestine.

In some embodiments, non-absorbed PL A₂ inhibitors of the invention comprise an art-known PL A₂ inhibiting moiety. Art-know PL A₂ inhibiting moieties include, for example, small molecule inhibitors of phospholipase A2, such as FPL 67047XX and/or MJ99. Other phospholipase inhibitors useful in the practice of the methods of this invention include arachidonic acid analogues (e.g., arachidonyl trifluoromethyl ketone, methylarachidonyl fluorophosphonate, and palmitoyl trifluoromethyl ketone), benzensulfonamide derivatives, bromoenol lactone, p-bromophenyl bromide, bromophenacyl bromide, trifluoromethylketone, sialoglycolipids, proteoglycans, and the like, as well as phospholipase A2 inhibitors disclosed in WO 03/101487, incorporated herein by reference.

Art-know PL A₂ inhibiting moieties useful in this invention also include, for example, phospholipid analogs and structures developed to target secreted PL A₂, for example, for indications such as obstructive respiratory disease (including asthma), colitis, Crohn's disease, central nervous system insult, ischemic stroke, multiple sclerosis, contact dermatitis, psoriasis, cardiovascular disease (including arteriosclerosis), autoimmune disease, and other inflammatory states.

Phospholipid analogs useful as phospholipase inhibiting moieties of some phospholipase inhibitors of this invention include structural analogs of a phospholipid substrate and/or its transition state, which can comprise one or more classes of compounds known in the art to resemble phospholipid substrates and/or their transition states, preferably resembling their polar head groups rather than their long chain hydrophobic groups. Such analog inhibitors can include, for example, compounds disclosed in Gelb M., Jain M., Berg O., Progress in Surgery, Principles of inhibition of phospholipase A2 and other interfacial enzymes, 1997, 24:123-129, for example, see Table 1 therein, incorporated herein by reference. Examples of PL A₂ inhibiting moieties in some preferred embodiments are provided below:

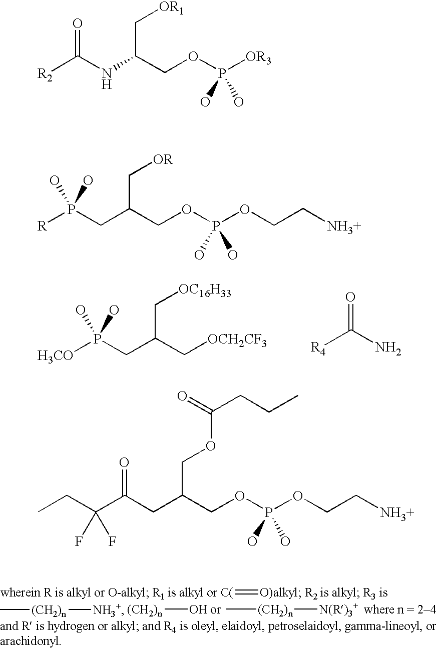

Phospholipid analogs useful as phospholipase inhibiting moieties of some phospholipase inhibitors of this invention also include phosphonate-containing compounds, such as those disclosed in Lin et al, J. Am. Chem. Soc., 115 (10) 1993, preferably the compounds represented by the structures provided below:

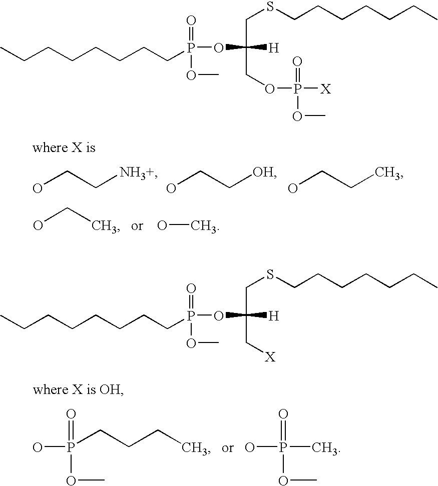

Transition state analogs useful as phospholipase inhibiting moieties of some phospholipid inhibitors of the present invention include one or more compounds taught in Jain, M et al., Biochemistry, 1991, 30:10256-10268, for example, see Tables IV, V and VI therein, incorporated herein by reference. In some preferred embodiments, inhibitors of the present invention comprise a moiety derived from modified glycerol backbone (see, for example, table VI of Jain, 1991), which have proven to be potent inhibitors of pancreatic PL A₂, including, for example, the structures illustrated below:

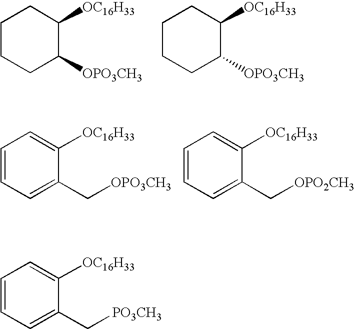

In some preferred embodiments, described below, the phospholipase-A2 inhibitor (or inhibiting moiety) can comprise indole compounds or indole-related compounds.

In general, therefore, preferred embodiments of the various aspects of the invention, the phospholipase inhibitor (or inhibiting moiety) can comprise a substituted organic compound (or moiety derived from a substituted organic compound) having a fused five-member ring and six-member ring (or as a pharmaceutically-acceptable salt thereof). Preferably, the inhibitor (or inhibiting moiety) also comprises substituent groups effective for imparting phospholipase-A2 inhibiting functionality to the inhibitor (or inhibiting moiety), and preferably phospholipase-A2 IB inhibiting functionality. Preferably the phospholipase inhibitor (inhibiting moiety) is a fused five-member ring and six-member ring having one or more heteroatoms (e.g., nitrogen, oxygen, sulfur) substituted within the ring structure of the five-member ring, within the ring structure of the six-member ring, or within the ring structure of each of the five-member and six-member rings (or as a pharmaceutically-acceptable salt thereof). Again preferably, the inhibitor (or inhibiting moiety) can comprise substituent groups effective for imparting phospholipase inhibiting functionality to the moiety.

As demonstrated in Example 10 (including related Examples 10A through 10C), substituted organic compounds (or moieties derived therefrom) having such fused five-member ring and six-member ring are effective phospholipase-2A I inhibitors, with phenotypic effects approaching and/or comparable to the effect of genetically deficient PLA2 (−/−) mice. Moreover, such compound (or moieties derived therefrom) are effective in treating conditions such as weight-related conditions, insulin-related conditions, and cholesterol-related conditions, including in particular conditions such as obesity, diabetes mellitus, insulin resistance, glucose intolerance, hypercholesterolemia and hypertriglyceridemia.

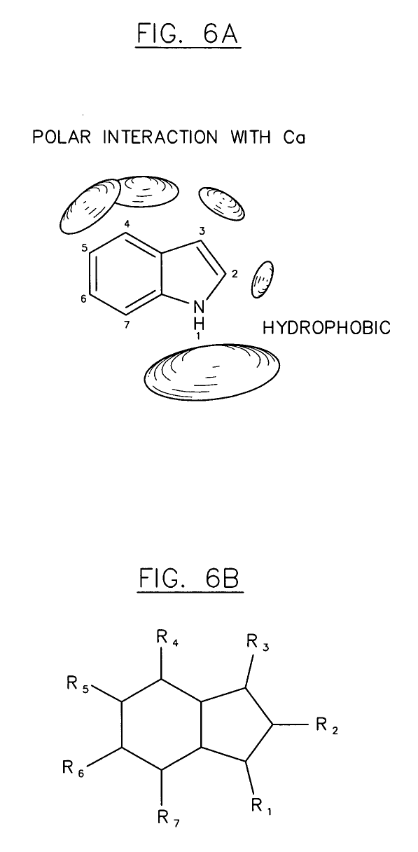

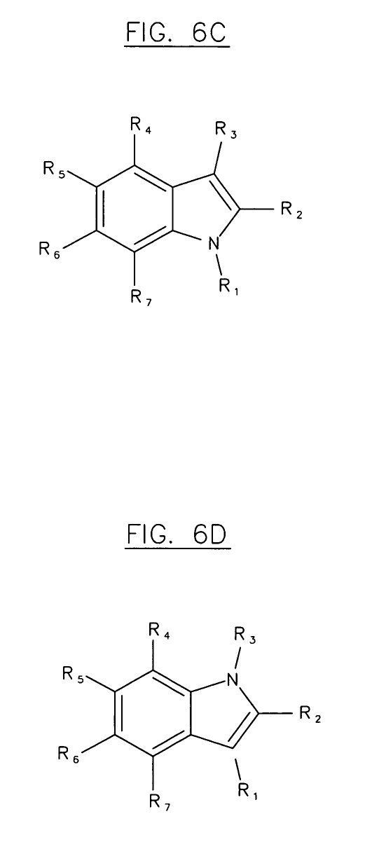

Although a particular compound was evaluated in-vivo in the study described in Example 10, namely the compound 2-(3-(2-amino-2-oxoacetyl)-1-(biphenyl-2-ylmethyl)-2-methyl-1H-indol-4-yloxy)acetic acid, shown in FIG. 5, the results of this study support a more broadly-defined invention, because the inhibitive effect can be realized and understood through structure-activity-relationships as described in detail hereinafter. Briefly, without being bound by theory not specifically recited in the claims, compounds comprising the fused five-membered and six-membered rings have a structure that advantageously provides an appropriate bond-length and bond-angles for positioning substituent groups—for example at positions 3 and 4 of an indole-compound as represented in FIG. 6A, and at the —R₃ and —R₄ positions of the indole-related compounds comprising fused five-membered and six-membered rings as represented in FIG. 6B. Mirror-image analogues of such indole compounds and of such indole-related compounds also can be used in connection with this invention, as described below.

In particularly preferred embodiments, the phospholipase-A2 inhibiting moiety can comprise a fused five-membered ring and six-membered ring as a compound (or as a pharmaceutically-acceptable salt thereof), represented by the following formula (I): 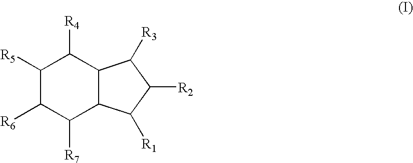
wherein the core structure can be saturated (as shown above) or unsaturated (not shown), and wherein R₁ through R₇ are independently selected from the group consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof; and additionally or alternatively, wherein R₁ through R₇ can optionally comprise, independently selected additional rings between two adjacent substitutents, with such additional rings being independently selected 5-, 6-, and/or 7-member rings which are carbocyclic rings, heterocyclic rings, and combinations thereof.

As used generally herein, including as used in connection with R₁ through R₇ in the indole-related compound shown above:

an amine group can include primary, secondary and tertiary amines;

a halide group can include fluoro, chloro, bromo, or iodo;

a carbonyl group can be a carbonyl moiety having a further substitution (defined below) as represented by the formula

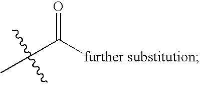

an acidic group can be an organic group as a proton donor and capable of hydrogen bonding, non-limiting examples of which include carboxylic acid, sulfate, sulfonate, phosphonates, substituted phosphonates, phosphates, substituted phosphates, 5-tetrazolyl,

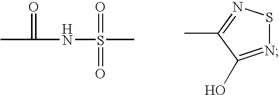

an alkyl group by itself or as part of another substituent can be a substituted or unsubstituted straight or branched chain hydrocarbon such as methyl, ethyl, n-propyl, isopropyl, n-butyl, tertiary butyl, sec-butyl, n-pentyl, n-hexyl, decyl, dodecyl, or octadecyl;

an alkenyl group by itself or in combination with other group can be a substituted or unsubstituted straight chain or branched hydrocarbon containing unsaturated bonds such as vinyl, propenyl, crotonyl, isopentenyl, and various butenyl isomers;

a carbocyclic group can be a substituted or unsubstituted, saturated or unsaturated, 5- to 14-membered organic nucleus whose ring forming atoms are solely carbon atoms, including cycloalkyl, cycloalkenyl, phenyl, spiro[5.5]undecanyl, naphthyl, norbornanyl, bicycloheptadienyl, tolulyl, xylenyl, indenyl, stilbenyl, terphenylyl, diphenylethylenyl, phenyl-cyclohexenyl, acenaphthylenyl, and anthracenyl, biphenyl, and bibenzylyl;

a heterocyclic group can be monocyclic or polycyclic, saturated or unsaturated, substituted or unsubstituted heterocyclic nuclei having 5 to 14 ring atoms and containing from 1 to 3 hetero atoms selected from the group consisting of nitrogen, oxygen or sulfur, including pyrrolyl, pyrrolodinyl, piperidinyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, dibenzothiophenyl, indazolyl, imidazo pyridinyl, benzotriazolyl, anthranilyl, 1,2-benzisoxazolyl, benzoxazolyl, benzothiazolyl, purinyl, pyridinyl, dipyridylyl. phenylpyridinyl, benzylpyridinyl, pyrimidinyl, phenylpyrimidinyl, pyrazinyl, 1,3,5-triazinyl, quinolinyl, phthalazinyl, quinazolinyl, morpholino, thiomorpholino, homopiperazinyl, tetrahydrofuranyl, tetrahydropyranyl, oxacanyl, 1,3-dioxolanyl, 1,3-dioxanyl, 1,4-dioxanyl, tetrahydrothiophenyl, pentamethylenesulfadyl, 1,3-dithianyl, 1,4-dithianyl, 1,4-thioxanyl, azetidinyl, hexamethyleneiminium, heptamethyleneiminium, piperazinyl and quinoxalinyl;

an acylamino group can be an acylamino moiety having two further substitutions (defined below) as represented by the formula: 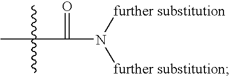
an oximyl group can be an oximyl moiety having two further substitutions (defined below) as represented by the formula:

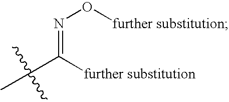

a hydrazyl group can be a hydrazyl moiety having three further substitutions (defined below) as represented by the formula:

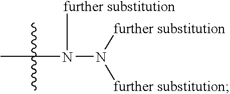

a substituted substitution group combines one or more of the listed substituent groups, preferably through moieties that include for example

an -oxygene-alkyl-acidic moiety such as

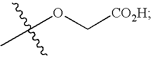

a -carbonyl-acyl amino-hydrogen moiety such as

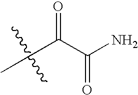

an -alkyl-carbocyclic-alkenyl moiety such as

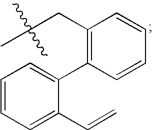

a -carbonyl-alkyl-thiol moiety such as

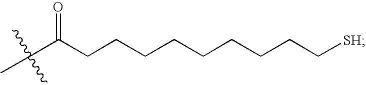

an -amine-carbonyl-amine moiety such as

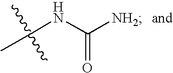

a further substitution group can mean a group selected from hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof.

Particularly preferred substituent groups R₁ through R₇ for such indole-related compounds are described below in connection with preferred indole-compounds.

In preferred embodiments, the phospholipase-A2 inhibiting moiety can comprise an indole compound (e.g., an indole-containing compound or compound containing an indole moiety), such as a substituted indole moiety. For example, in such embodiment, the indole-containing compound can be a compound represented by the formulas II, III (considered left to right as shown): 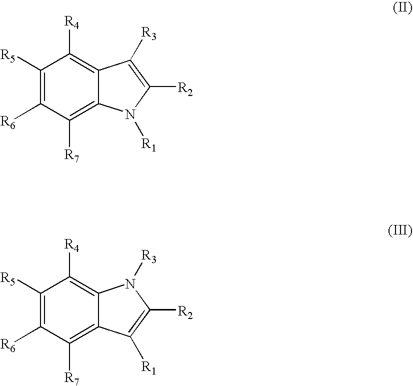
wherein R₁ through R₇ are independently selected from the groups consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof; and additionally or alternatively, wherein R₁ through R₇ can optionally, and independently form additional rings between two adjacent substitutents with such additional rings being 5-, 6-, and 7-member ring selected from the group consisting of carbocyclic rings, heterocyclic rings and combinations thereof.

In some embodiments, the phospholipase-A2 inhibiting moiety can comprise an azaindole compound (e.g., an azaindole-containing compound or compound containing an azaindole moiety), such as a substituted azaindole moiety. For example, in such embodiment, the azaindole-containing compound can be a compound represented by a formula selected from 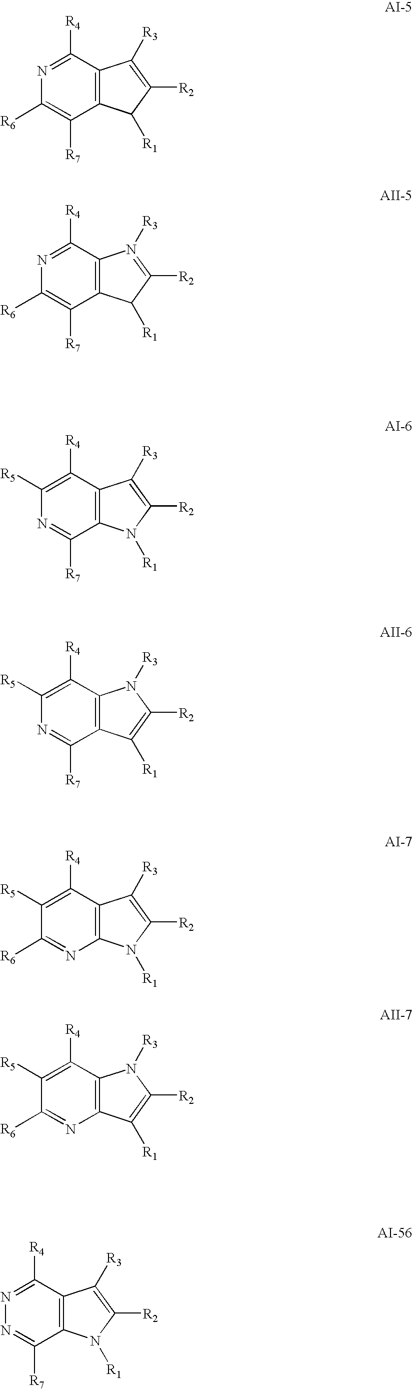 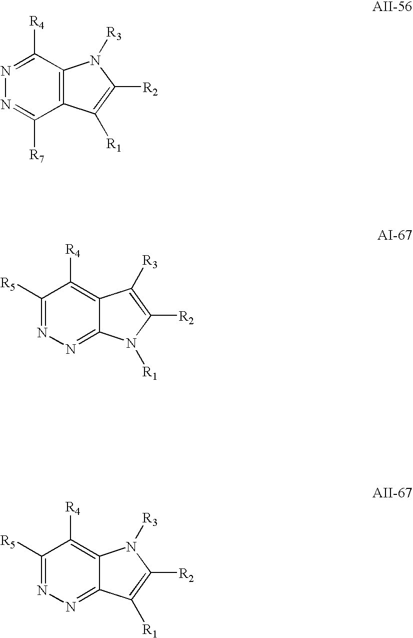
wherein with respect to each of the formulas, R₁ through R₇ each being independently selected from the group consisting of hydrogen, halide, oxygen, sulfur, phosphorus, hydroxyl, amine, thiol, alkyl, substituted alkyl, alkenyl, substituted alkenyl, alkynyl, substituted alkynyl, ether, carbonyl, acidic, carboxyl, ester, amide, carbocyclic, heterocyclic, acylamino, oximyl, hydrazyl and moieties comprising combinations thereof, optionally and preferably with respect to each of the formulas, R₁ through R₇ are independently selected from the groups consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof.

Some indole compounds (or azaindole compounds) can comprise additional rings, as noted. For example, some indole compounds having additional rings include, for example, those compounds represented as formulas IVa through IVf (considered left to right in top row as IVa, IVb, IVc, and considered left to right bottom row as IVd, IVe and IVf, as shown):

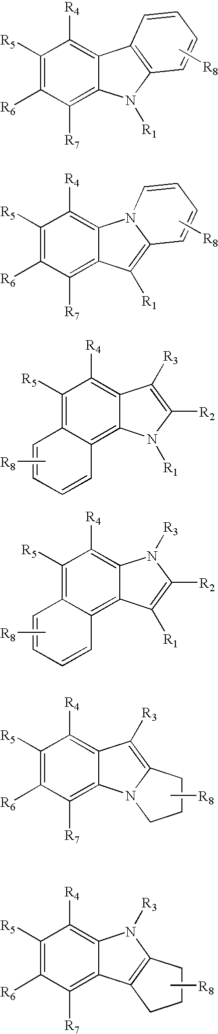

Generally, the various types of substituent groups, including carbonyl, acidic, alkyl, alkenyl, carbocyclic, heterocyclic, acylamino, oximyl, hydrazyl, substituted substitution group, can be as defined above in connection with the indole-related compounds (including indole and azaindole compounds) having fused five-membered and six-membered rings.

In each of the embodiments of the invention, including for those compounds that are indole-related compounds having fused five-membered and six-membered rings, and for the indole compounds, preferred substitutent groups can be as described in the following paragraphs.

Preferred R₁ is selected from the following groups: hydrogen, oxygen, sulfur, amine, halide, hydroxyl (—OH), thiol (—SH), carbonyl, acidic, alkyl, alkenyl, carbocyclic, heterocyclic, substituted substitution group and combinations thereof. Particularly preferred R₁ is selected from the following groups: hydrogen, halide, thiol (—SH), carbonyl, acidic, alkyl, alkenyl, carbocyclic, substituted substitution group and combinations thereof. R₁ is especially preferably selected from the group consisting of alkyl, carbocyclic and substituted substitution group. The substituted substitution group for R₁ are especially preferred compounds or moieties such as:

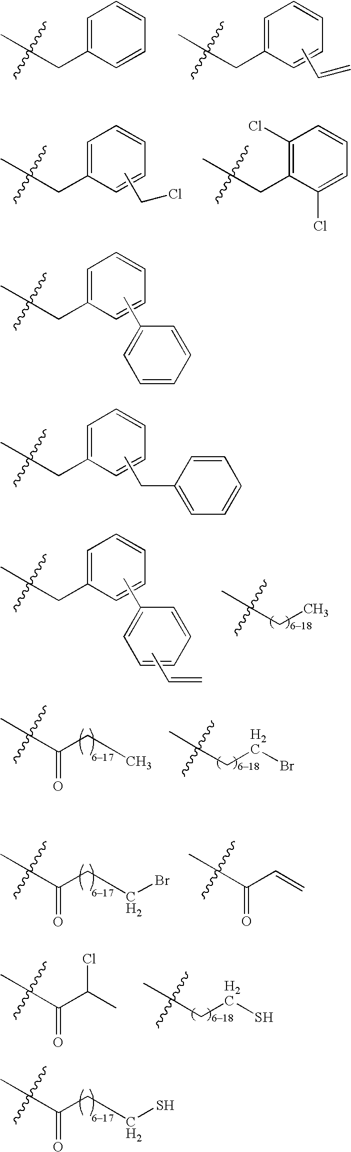

Preferred R₂ is selected from the following groups: hydrogen, oxygen, halide, carbonyl, alkyl, alkenyl, carbocyclic, substituted substitution group, and combinations thereof. Particularly preferred R₂ is selected from the following groups: hydrogen, halide, alkyl, alkenyl, carbocyclic, substituted substitution group, and combinations thereof. R₂ is preferably selected from the group consisting of halide, alkyl and substituted substitution group. The substituted substitution group for R₂ are especially preferred compounds or moieties such as:

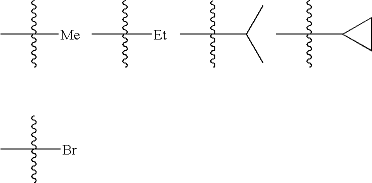

Preferred R₃ is selected from the following groups: hydrogen, oxygen, sulfur, amine, hydroxyl (—OH), thiol (—SH), carbonyl, acidic, alkyl, heterocyclic, acylamino, oximyl, hydrazyl, substituted substitution group and combinations thereof. Particularly preferred R₃ is selected from the following groups: hydrogen, oxygen, amine, hydroxyl (—OH), carbonyl, alkyl, acylamino, oximyl, hydrazyl, substituted substitution group and combinations thereof. R₃ is preferably selected from the group consisting of carbonyl, acylamino, oximyl, hydrazyl, and substituted substitution group. The substituted substitution group for R₃ are especially preferred compounds or moieties such as:

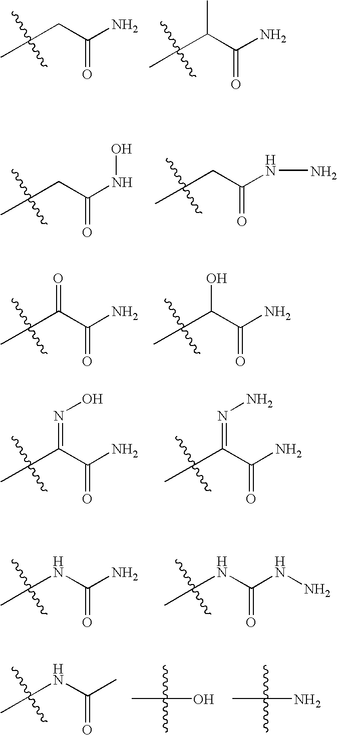

Preferred R₄ and R₅ are independently selected from the following groups: hydrogen, oxygen, sulfur, phosphorus, amine, hydroxyl (—OH), thiol (—SH), carbonyl, acidic, alkyl, alkenyl, heterocyclic, acylamino, oximyl, hydrazyl, substituted substitution group and combinations thereof. Particularly preferred R₄ and R₅ are independently selected from the following groups: hydrogen, oxygen, sulfur, amine, acidic, alkyl, substituted substitution group and combinations thereof. R₄ and R₅ are each preferably independently selected from the group consisting of oxygen, hydroxyl (—OH), acidic, alkyl, and substituted substitution group. The substituted substitution group for R₄ and for R₅ are especially preferred compounds or moieties such as:

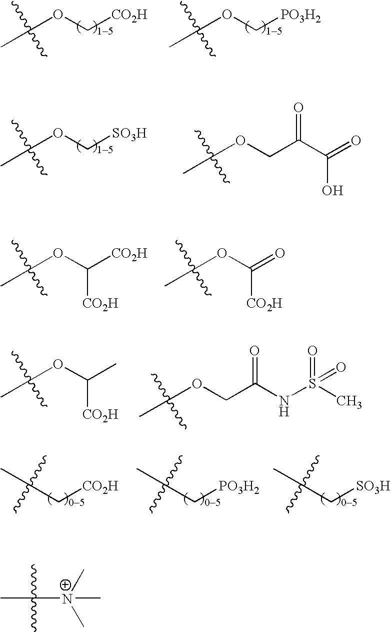

Preferred R₆ is selected from the following groups hydrogen, oxygen, amine, halide, hydroxyl (—OH), acidic, alkyl, carbocyclic, acylamino, substituted substitution group and combinations thereof. Particularly preferred R₆ is selected from the following groups: hydrogen, oxygen, amine, halide, hydroxyl (—OH), acidic, alkyl, acylamino, substituted substitution group and combinations thereof. R₆ is preferably selected from the group consisting of amine, acidic, alkyl, and substituted substitution group. The substituted substitution group for R₆ are especially preferred compounds or moieties such as:

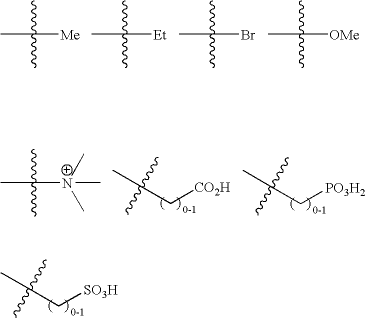

Preferred R₇ is selected from the following groups: hydrogen, oxygen, sulfur, amine, halide, hydroxyl (—OH), thiol (—SH), carbonyl, acidic, alkyl, alkenyl, carbocyclic, heterocyclic, substituted substitution group and combinations thereof. Particularly preferred R₇ is selected from the following groups: hydrogen, halide, thiol (—SH), carbonyl, acidic, alkyl, alkenyl, carbocyclic, substituted substitution group and combinations thereof. R₇ is preferably selected from the groups consisting of carbocyclic and substituted substitution group. The substituted substitution group for R₇ are especially preferred compounds or moieties such as:

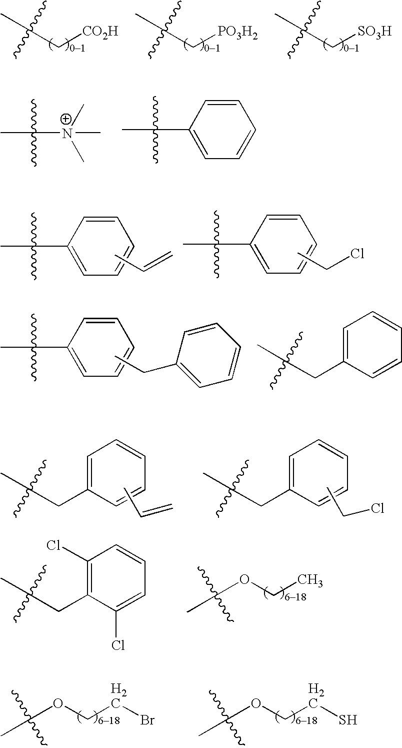

The aforementioned preferred selections for each substituent group R₁ through R₇ can be combined in each variation and permutation. In certain, preferred embodiments, for example, the inhibitor of the invention can comprise substituent groups wherein R₁ through R₇ are as follows: R₁ is preferably selected from the group consisting of alkyl, carbocyclic and substituted substitution group; R₂ is preferably selected from the group consisting of halide, alkyl and substituted substitution group; R₃ is preferably selected from the group consisting of carbonyl, acylamino, oximyl, hydrazyl, and substituted substitution group; R₄ and R₅ are each preferably independently selected from the group consisting of oxygen, hydroxyl (—OH), acidic, alkyl, and substituted substitution group; R₆ is preferably selected from the group consisting of amine, acidic, alkyl, and substituted substitution group; and R₇ is preferably selected from the groups consisting of carbocyclic and substituted substitution group.

In especially preferred embodiments, R₃ is a moiety represented by formula (C3-I or C3-II) 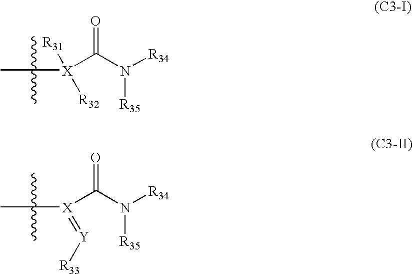
with: X being selected from the group consisting of O, C and N; R₃₁ being optional, and if present being selected from the group consisting of hydrogen, halide, hydroxyl and cyano; R₃₂ being optional, and if present being selected from the group consisting of hydrogen, halide, hydroxyl, and cyano; Y being selected from the group consisting of O, S, and N; R₃₃ being optional, and if present being selected from the group consisting of hydrogen, hydroxyl, C₁-C₆ alkyl, substituted C₁-C₆ alkyl, C₁-C₆ alkoxyl and substituted C₁-C₆ alkoxyl; and R₃₄ and R₃₅ each being independently selected from the group consisting of hydrogen, hydroxyl, alkoxyl, alkyl, substituted alkyl, amine, and alkylsulfonyl.

In some preferred embodiments, R₃ can preferably be a moiety represented by formula (C3-I-A or C3-II-A) 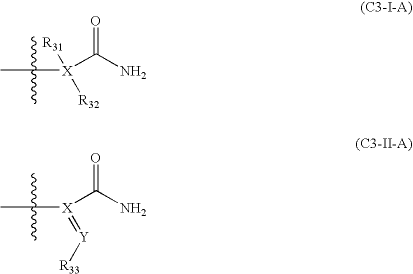
with: X being selected from the group consisting of O, C and N; R₃₁ being optional, and if present being selected from the group consisting of hydrogen, halide, hydroxyl and cyano; R₃₂ being optional, and if present being selected from the group consisting of hydrogen, halide, hydroxyl, and cyano; Y being selected from the group consisting of O, S, and N; R₃₃ being optional, and if present being selected from the group consisting of hydrogen, hydroxyl, C₁-C₆ alkyl, substituted C₁-C₆ alkyl, C₁-C₆ alkoxyl and substituted C₁-C₆ alkoxy.

R₃ can most preferably be a moiety represented by a formula selected from the group consisting of

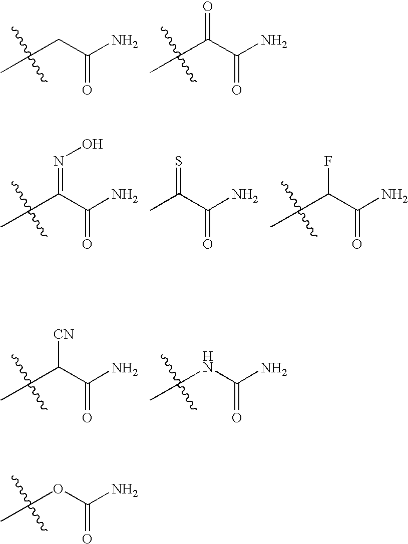

In especially preferred embodiments (including in embodiments with especially preferred R₃ as described in the immediately preceding paragraphs), R₄ can be a moiety selected from

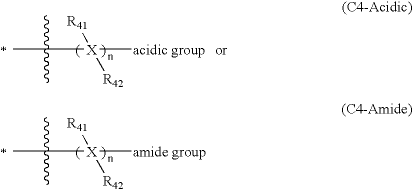

with as applicable and independently selected for each formula: n being an integer ranging from 1 to 5; and for each n: X being independently selected from the group consisting of C, O, S, and N; and R₄₁ and R₄₂ each being optional, but if present being independently selected from the group consisting of hydrogen, halide, alkyl, substituted alkyl, phenyl, aryl, amine, alkoxyl, alkylysulfonyl, alkylphosphonyl, alkylcarbonyl, carboxyl, phosphonic, sulfonic, carboxamide, and cyano.

In particular, R₄ can be an acidic substituent, and can preferably be a moiety represented by formula selected from (C4-I-A), (C4-I-B) and (C4-I-C) 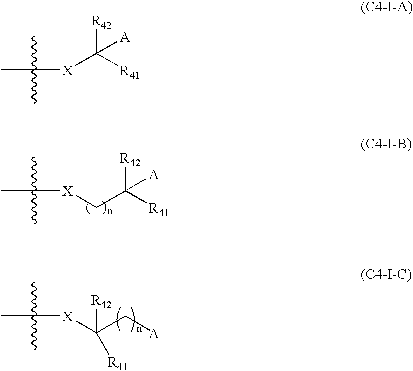
in each case, independently selected for each of C4-1A, C4-I-B and C4-I-C above with: n being an integer ranging from 0 to 5, and preferably ranging from 0 to 3; X being selected from the group consisting of O, C and N; A being an acidic group; R₄₁ being selected from the group consisting of hydrogen, halide, hydroxyl and cyano; and R₄₂ being selected from the group consisting of (i) C₂-C₆ alkyl, (ii) C₂-C₆ alkyl substituted with one or more substituents selected from halide, hydroxyl and amine, (iii) halide, and (iv) carboxyl. Preferred R₄₂ is a moiety selected from C₂-C₄ alkyl and substituted C₂-C₄ alkyl. R₄₂ can be a moiety selected from C₂-C₄ alkyl and C₂-C₄ alkyl substituted with one or more substituents selected from halide, hydroxyl and amine. Especially preferred R₄₂ can be ethyl, propyl, isopropyl, isobutyl and tertbutyl.

Especially preferred R₄ can be a moiety represented by formula selected from the group consisting of

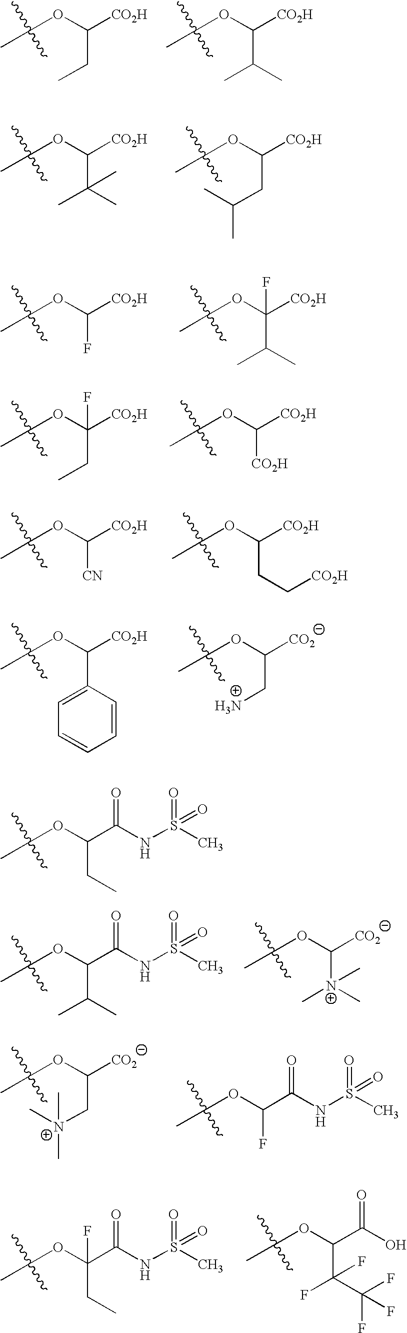

R₄ can additionally or alternatively be an amide substituent, and can be a moiety represented by formula selected from (C4-II-A), (C4-II-B), (C4-II-C) and (C4-II-D) 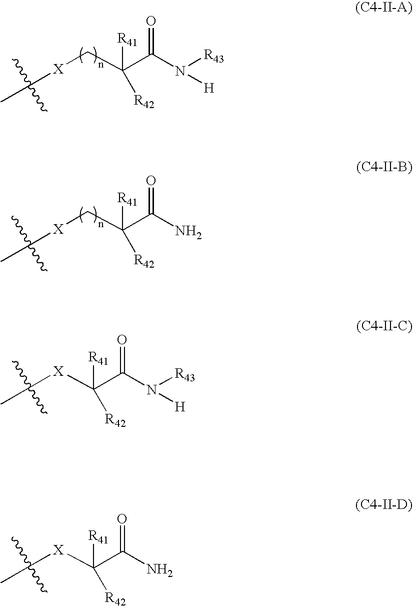
with as applicable and independently selected for each formula: n being an integer ranging from 0 to 5, preferably 0 to 3; X being selected from the group consisting of O, C, S and N; R₄₁ being selected from the group consisting of hydrogen, halide, hydroxyl, alkoxyl, alkyl, substituted alkyl, carboxyl, carboxamide, alkylcarbonyl, amine, alkylphosphonyl, alkylsulfonyl, sulfonic, phosphonic, and cyano; R₄₂ being selected from the group consisting of, halide, hydroxyl, alkoxyl, alkyl, substituted alkyl, carboxyl, carboxamide, alkylcarbonyl, amine, alkylphosphonyl, alkylsulfonyl, sulfonic, phosphonic, and cyano, and R₄₃ being selected from the group consisting of hydrogen, phenyl, aryl, C₁-C₆ alkyl, and C₁-C₆ alkyl substituted with a moiety selected from the group consisting of hydrogen, halide, hydroxyl, amine, sulfonic, phosphonic, and cyano.

R₄ can also (additionally or alternatively) be an amide substituent moiety represented by formula (C4-III-A), (C4-III-B), (C4-III-F) or (C4-III-G) 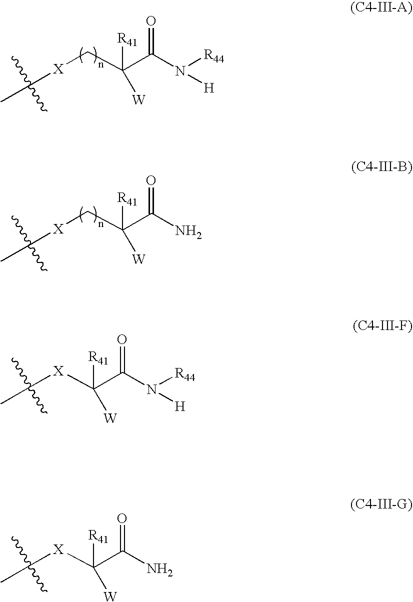
with independently selected for each formula, as applicable: n being an integer ranging from 0 to 5, preferably 0 to 3; X being independently selected from the group consisting of O, C, S and N; W being an electron withdrawing group; R₄₁ being selected from the group consisting of hydrogen, halide, hydroxyl, alkoxyl, alkyl, substituted alkyl, carboxyl, carboxamide, alkylcarbonyl, amine, alkylphosphonyl, alkylsulfonyl, sulfonic, phosphonic, and cyano; and (for formulas C4-III-A and C4-III-F) R₄₄ being selected from the group consisting of hydrogen, phenyl, aryl, hydroxyl, alkoxyl, alkylsulfonyl, alkylphosphonyl, amine, C₁-C₆ alkyl, and C₁-C₆ alkyl substituted with a moiety selected from the group consisting of hydrogen, halide, hydroxyl, amine, carboxyl, sulfonic, phosphonic, and cyano.

In some embodiments, R₄ can be a moiety represented by formula (C4-III-C) or (C4-III-H) 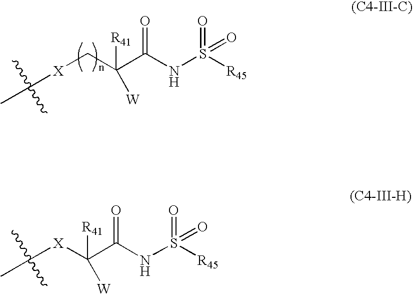
with as applicable, and independently selected for each formula: n being an integer ranging from 0 to 5, preferably 0 to 3; X being independently selected from the group consisting of O, C, S and N; W being an electron withdrawing group; R₄₁ being selected from the group consisting of hydrogen, halide, hydroxyl, alkoxyl, alkyl, substituted alkyl, carboxyl, carboxamide, alkylcarbonyl, amine, alkylphosphonyl, alkylsulfonyl, sulfonic, phosphonic, and cyano; and R₄₅ being selected from the group consisting of hydrogen, phenyl, aryl, hydroxyl, alkoxyl, alkylsulfonyl, alkylphosphonyl, amine, C₁-C₆ alkyl, and C₁-C₆ alkyl substituted with a moiety selected from the group consisting of hydrogen, halide, hydroxyl, amine, carboxyl, sulfonic, phosphonic, and cyano.

In some embodiments, R₄ can be a moiety represented by formula (C4-III-D) or (C4-III-J) 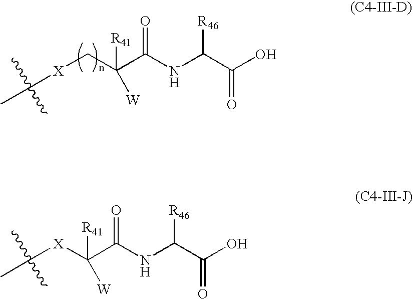
with as applicable, and independently selected for each formula: n being an integer ranging from 0 to 5, preferably 0 to 3; X being independently selected from the group consisting of O, C, S and N; W being an electron withdrawing group; R₄₁ being selected from the group consisting of hydrogen, halide, hydroxyl, alkoxyl, alkyl, substituted alkyl, carboxyl, carboxamide, alkylcarbonyl, amine, alkylphosphonyl, alkylsulfonyl, sulfonic, phosphonic, and cyano; and R₄₆ being selected from the group consisting of hydrogen, phenyl, aryl, alkylsulfonyl, alkylphosphonyl, C₁-C₆ alkyl, and C₁-C₆ alkyl substituted with a moiety selected from the group consisting of hydrogen, halide, hydroxyl, amine, carboxyl, sulfonic, phosphonic, and cyano.

In some embodiments, R₄ can be a moiety represented by formula (C4-III-E) or (C4-III-K) 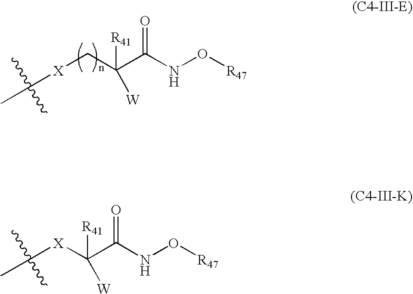
with as applicable, and independently for each formula: n being an integer ranging from 0 to 5, preferably 0 to 3; X being independently selected from the group consisting of O, C, S and N; W being an electron withdrawing group; R₄₁ being selected from the group consisting of hydrogen, halide, hydroxyl, alkoxyl, alkyl, substituted alkyl, carboxyl, carboxamide, alkylcarbonyl, amine, alkylphosphonyl, alkylsulfonyl, sulfonic, phosphonic, and cyano; and R₄₇ being selected from the group consisting of hydrogen, phenyl, aryl, C₁-C₆ alkyl, and C₁-C₆ alkyl substituted with a moiety selected from the group consisting of hydrogen, halide, hydroxyl, amine, carboxyl, sulfonic, phosphonic, and cyano.

In any of the aforementioned embodiments of formulas C4-III-A, -B, -C, -D, -E, -F, -G, -H, -J, -K, as applicable and in each case independently: R₄₁ is preferably selected from the group consisting of hydrogen, halide, haloalkyl, carboxyl, carboxamide, alkylcarbonyl, amine, alkyl alkylphosphonyl, alkylsulfonyl, sulfonic, phosphonic, and cyano; R₄₂ is preferably selected from the group consisting of halide, haloalkyl, carboxyl, carboxamide, alkylcarbonyl, amine, alkyl alkylphosphonyl, alkylsulfonyl, sulfonic, phosphonic, and cyano; R₄₃ is preferably selected from the group consisting of hydrogen, C₁-C₆ alkyl, and C₁-C₆ alkyl substituted with a moiety selected from the group consisting of hydrogen, hydroxyl, amine, sulfonic, and phosphonic; W is preferably selected from the group consisting of halide, hydroxyl, alkoxyl, haloalkyl, carboxyl, carboxamide, alkylcarbonyl, amine, alkylphosphonyl, alkylsulfonyl, sulfonic, phosphonic, and cyano; R₄₄ is preferably selected from the group consisting of hydrogen, hydroxyl, alkoxyl, alkylsulfonyl, C₁-C₆ alkyl, and C₁-C₆ alkyl substituted with a moiety selected from the group consisting of hydrogen, amine, carboxyl, sulfonic, and phosphonic; R₄₅ is preferably selected from the group consisting of C₁-C₆ alkyl substituted with a moiety selected from the group consisting of hydrogen, halide, hydroxyl, amine, carboxyl, sulfonic, phosphonic, and cyano; R₄₅ can be more preferably selected from the group consisting of C₁-C₃ alkyl substituted with a moiety selected from the group consisting of hydrogen, halide, hydroxyl, amine, carboxyl, sulfonic, phosphonic, and cyano; R₄₆ is preferably selected from the group consisting of C₁-C₆ alkyl substituted with a moiety selected from the group consisting of hydrogen, halide, hydroxyl, amine, carboxyl, sulfonic, phosphonic, and cyano. R₄₆ can be more preferably selected from the group consisting of C₁-C₃ alkyl substituted with a moiety selected from the group consisting of hydrogen, halide, hydroxyl, amine, carboxyl, sulfonic, phosphonic, and cyano; R₄₇ is preferably selected from the group consisting of C₁-C₆ alkyl substituted with a moiety selected from the group consisting of hydrogen, halide, hydroxyl, amine, carboxyl, sulfonic, phosphonic, and cyano; R₄₇ can be more preferably selected from the group consisting of C₁-C₃ alkyl substituted with a moiety selected from the group consisting of hydrogen, halide, hydroxyl, amine, carboxyl, sulfonic, phosphonic, and cyano.

In some embodiments, R₄ can be a moiety represented by a formula selected from the group consisting of 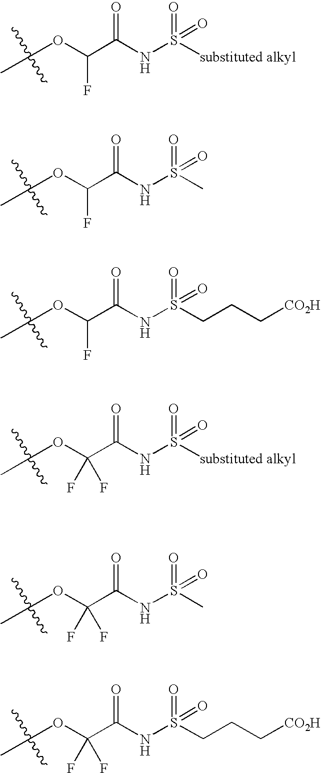
with: substituted alkyl being a C₁-C₆ alkyl substituted with a moiety selected from the group consisting of hydrogen, halide, hydroxyl, amine, carboxyl, sulfonic, phosphonic, and cyano.

In some embodiments, R₄ can be a moiety represented by a formula selected from the group consisting of 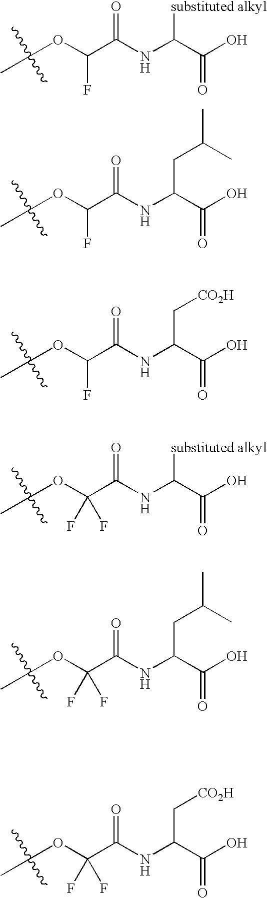
with: substituted alkyl being a C₁-C₆ alkyl substituted with a moiety selected from the group consisting of hydrogen, halide, hydroxyl, amine, carboxyl, sulfonic, phosphonic, and cyano.

In some embodiments, R₄ is a moiety represented by a formula selected from the group consisting of 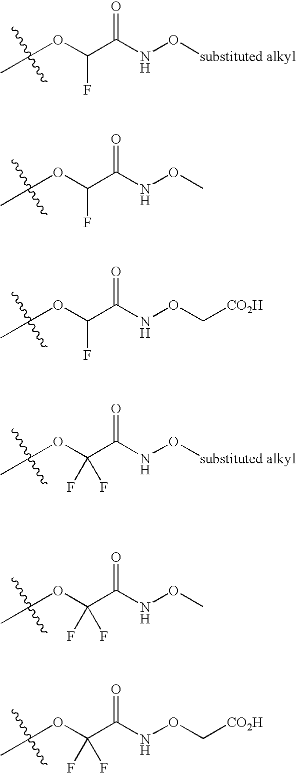
with: substituted alkyl being a C₁-C₆ alkyl substituted with a moiety selected from the group consisting of hydrogen, halide, hydroxyl, amine, carboxyl, sulfonic, phosphonic, and cyano.

In especially preferred embodiments, R₄ can be a moiety represented by a formula selected from the group consisting of

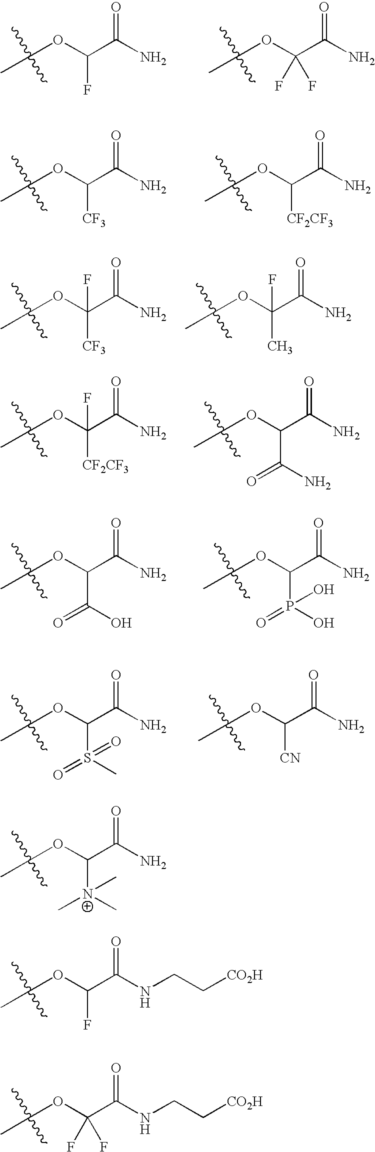

In some especially preferred embodiments (including in embodiments with preferred R₃ and R₄ as described in the immediately preceding paragraphs), R₂ can be selected from the group consisting of hydrogen, halide, hydroxyl, C₁-C₃ alkyl, substituted C₁-C₃ alkyl, and cyano. R₂ can preferably be selected from the group consisting of hydrogen, halide, and C₁-C₃ alkyl. R₂ can be a moiety represented by a formula selected from the group consisting of

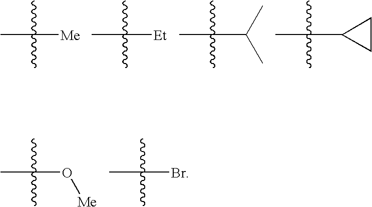

In some especially preferred embodiments, (including in embodiments with preferred R₂, R₃ and R₄ as described in the immediately preceding paragraphs), R₅ can be selected from the group consisting of hydrogen, halide, hydroxyl, C₁-C₃ alkyl and cyano. R₅ can preferably be selected from the group consisting of hydrogen, chloride, fluoride, hydroxyl, methyl and cyano.

In some especially preferred embodiments (including in embodiments with preferred R₂, R₃, R₄ and R₅ as described in the immediately preceding paragraphs), R₁, R₆ and R₇ can each being independently selected from the group consisting of hydrogen, halide, hydroxyl, amine, carboxyl, phosphonic, sulfonic, alkyl, substituted alkyl, alkoxyl, substituted alkoxyl, alkyl carbonyl, substituted alkyl carbonyl, carbocyclic, heterocyclic, and moieties comprising combinations thereof.

R₁ can preferably be selected from the group consisting of C₄-C₃₆ alkyl, substituted C₄-C₃₆ alkyl, carbocyclic, heterocyclic, alkyl carbonyl, substituted alkyl carbonyl, and moieties comprising combinations thereof. R₁ can be selected from the group consisting of C₄-C₃₆ alkyl, substituted C₄-C₃₆ alkyl, carbocyclic, and moieties comprising combinations thereof.

R₁ can be a moiety represented by a formula selected from the group consisting of

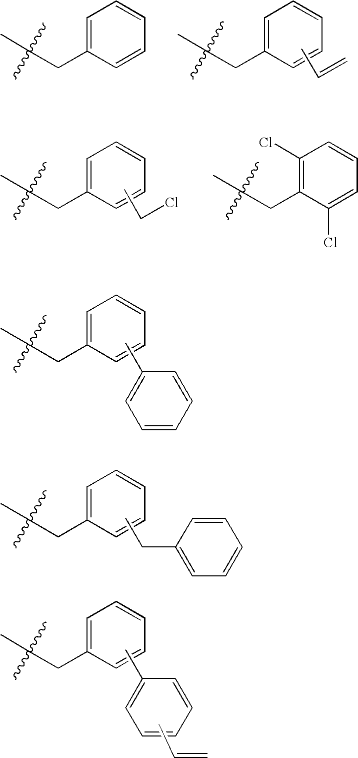

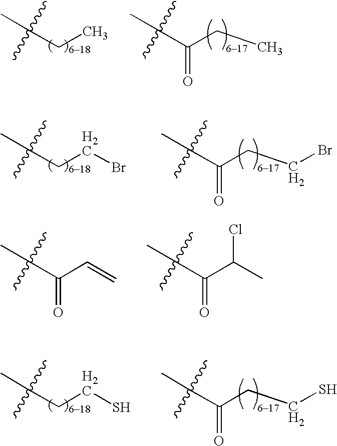

R₁ can be a moiety comprising a multifunctional bridge moiety or linked to a multifunctional bridge moiety.

R₆ can be selected from the group consisting of hydrogen, halide, amine, C₁-C₃ alkyl, substituted C₁-C₃ alkyl, acidic, and moieties comprising combinations thereof. R₆ can be a moiety represented by a formula selected from the group consisting of

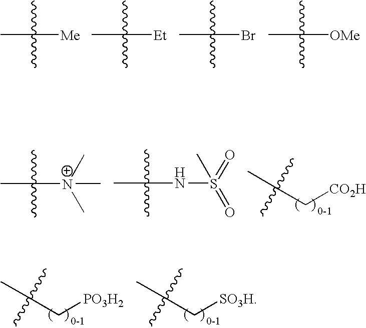

R₆ can be a moiety comprising a multifunctional bridge moiety.

R₇ can be selected from the group consisting of C₄-C₃₆ alkyl, substituted C₄-C₃₆ alkyl, carbocyclic, heterocyclic, alkyl carbonyl, substituted alkyl carbonyl, and moieties comprising combinations thereof. R₇ can be selected from the group consisting of C₄-C₃₆ alkyl, substituted C₄-C₃₆ alkyl, carbocyclic, and moieties comprising combinations thereof. R₇ can be a carbocyclic moiety.

R₇ can be a moiety represented by a formula selected from the group consisting of

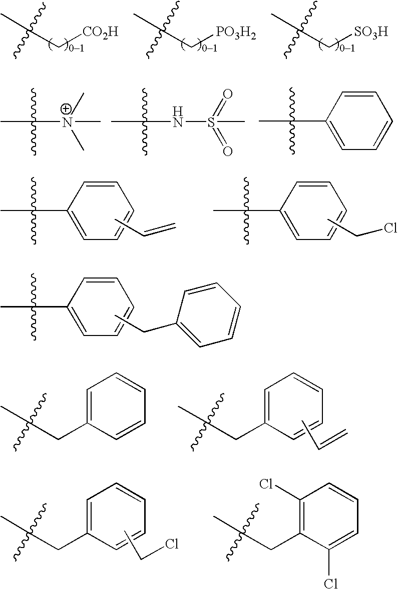

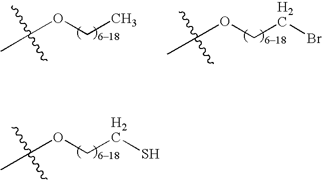

R₇ can be a moiety comprising a multifunctional bridge moiety.

Certain indole glyoxamides are particularly useful as PL A₂ inhibiting moieties in some embodiments. Specifically [2-(3-(2-amino-2-oxoacetyl)-1-(biphenyl-2-ylmethyl)-2-methyl-1H-indol-4-yloxy)acetic acid], shown in FIG. 2, alternatively referred to herein as ILY-4001 and/or as methyl indoxam has been found to be an effective phospholipase inhibitor or inhibiting moiety. This indole compound is represented by the structure below, as formula (V):

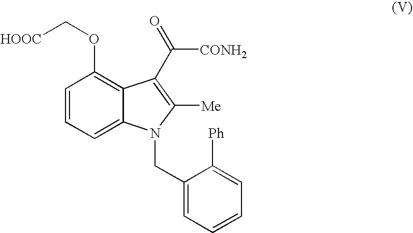

This compound has been shown, based on in-vitro assays, to have phospholipase activity for a number of PLA2 classes, and is a strong inhibitor of mouse and human PLA2IB enzymes in vitro (Singer, Ghomashchi et al. 2002; Smart, Pan et al. 2004). This indole compound was synthesized (See, Example 1A) and was evaluated in-vivo for phospholipase-A2 inhibition in a mice model. (See, Example 10, including Examples 10A through 10C). This indole compound was characterized with respect to inhibition activity, absorption and bioavailability. (See, Example 1B, including Examples 1B-1, 1B-2 and 1B-3).

Bioavailability of this compound can be reduced, and reciprocally, lumen-localization can be improved, according to this second general embodiment of the invention, for example, by covalently linking this indole moiety to a polymer. (See, for example, Example 1D).

Other compounds are also particularly preferred as phospholipase inhibiting moieties for use in connection with any embodiment of the invention. In particular, for example, the phospholipase inhibiting moiety can be a moiety represented by a formula selected from

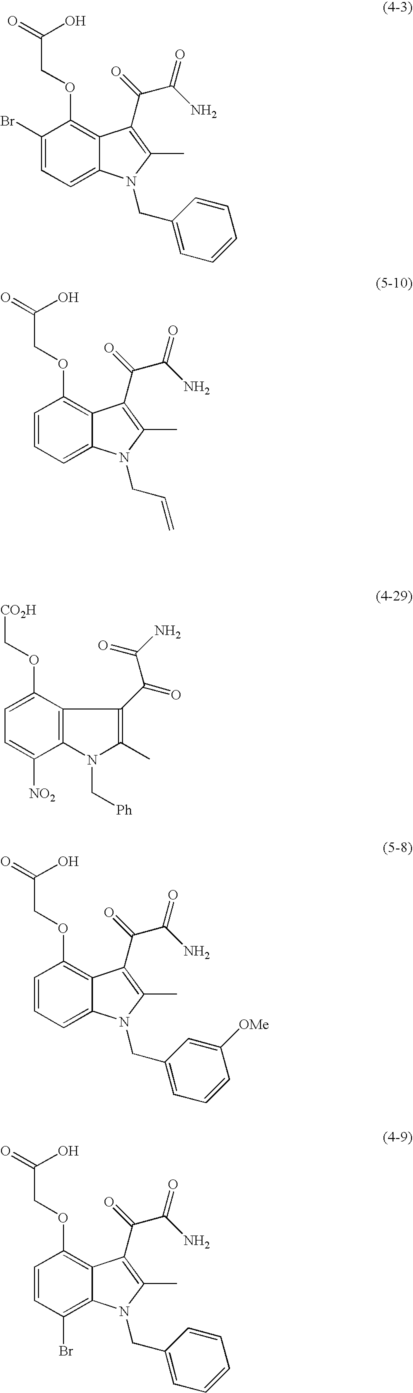

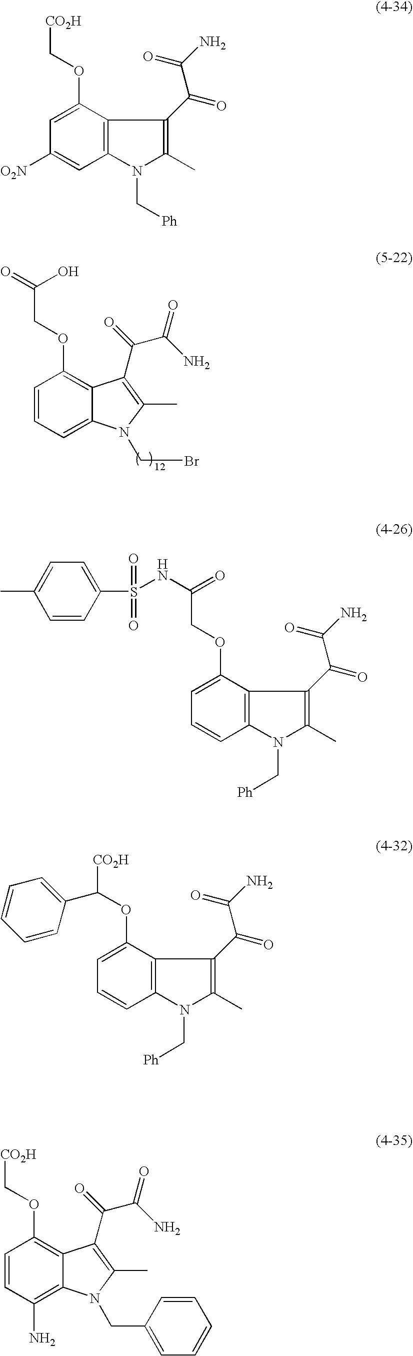

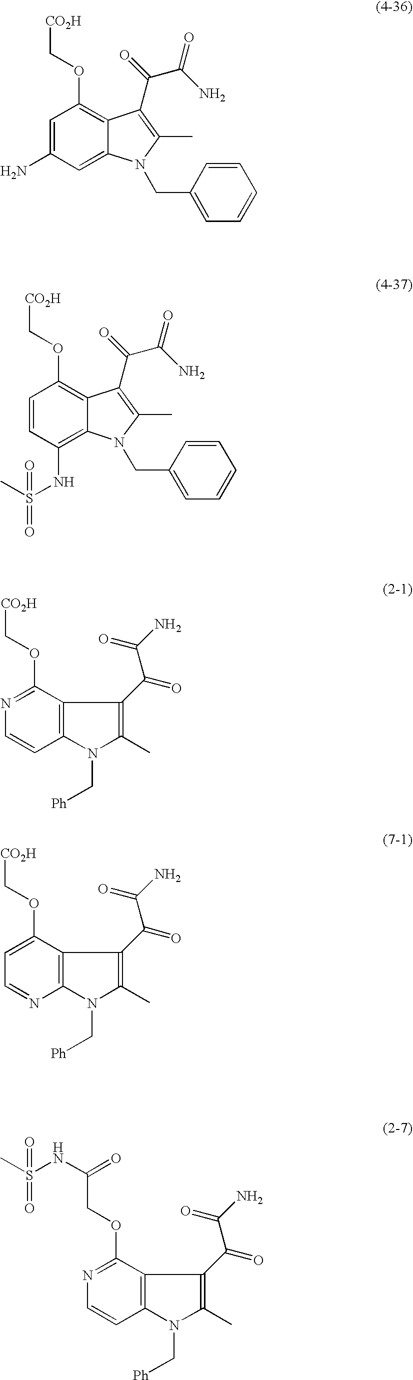

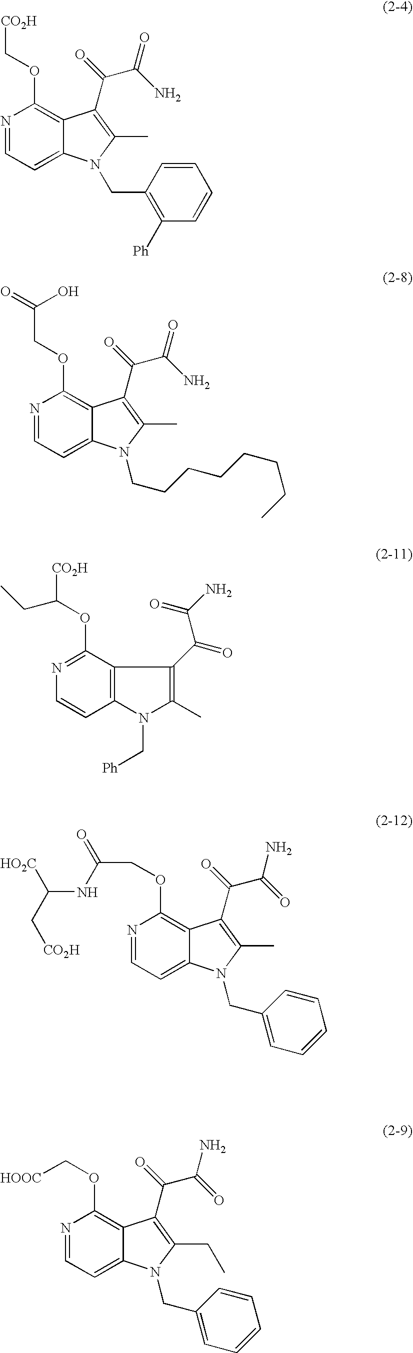

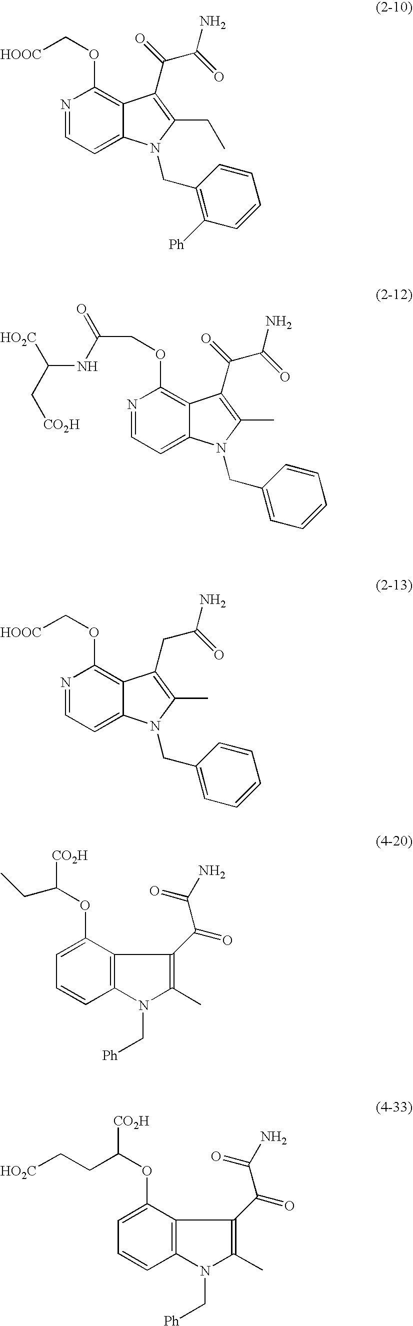

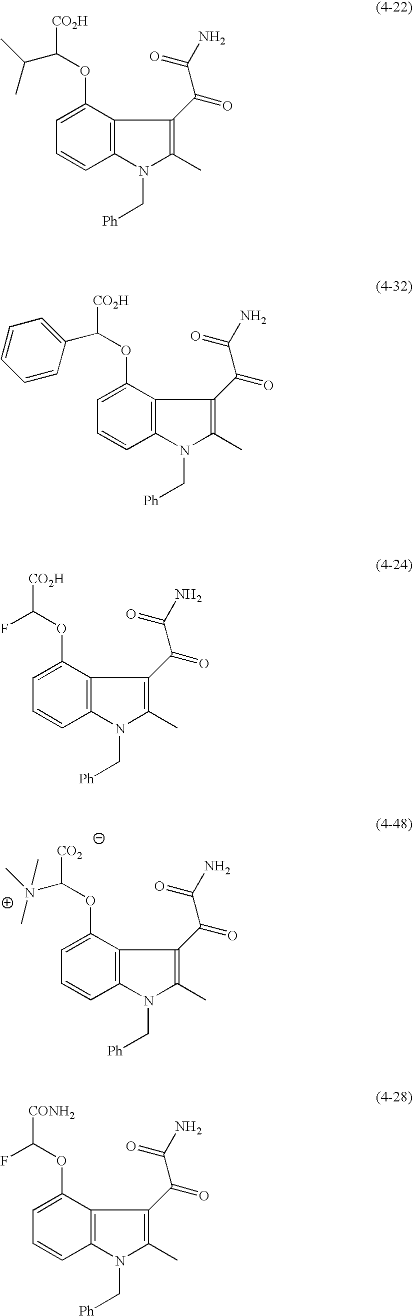

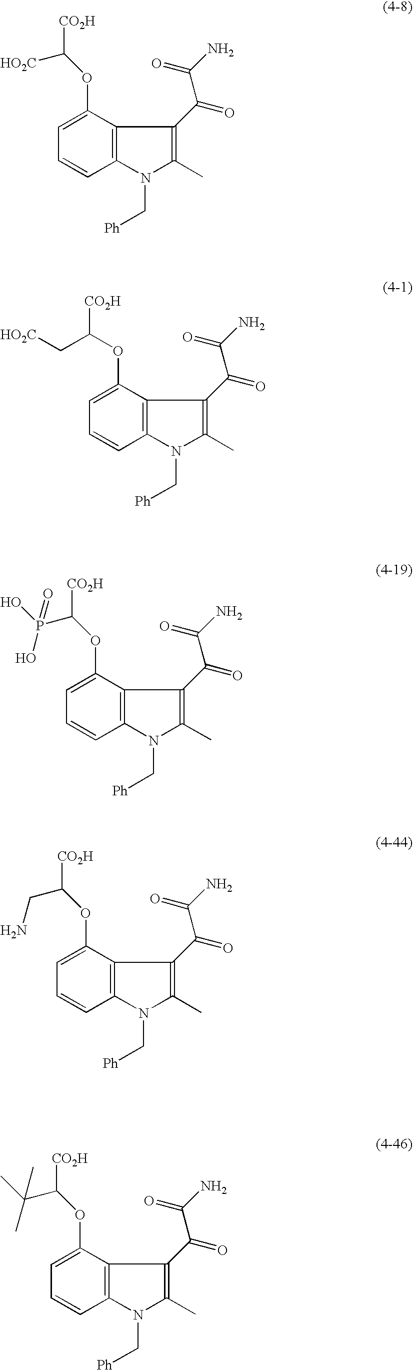

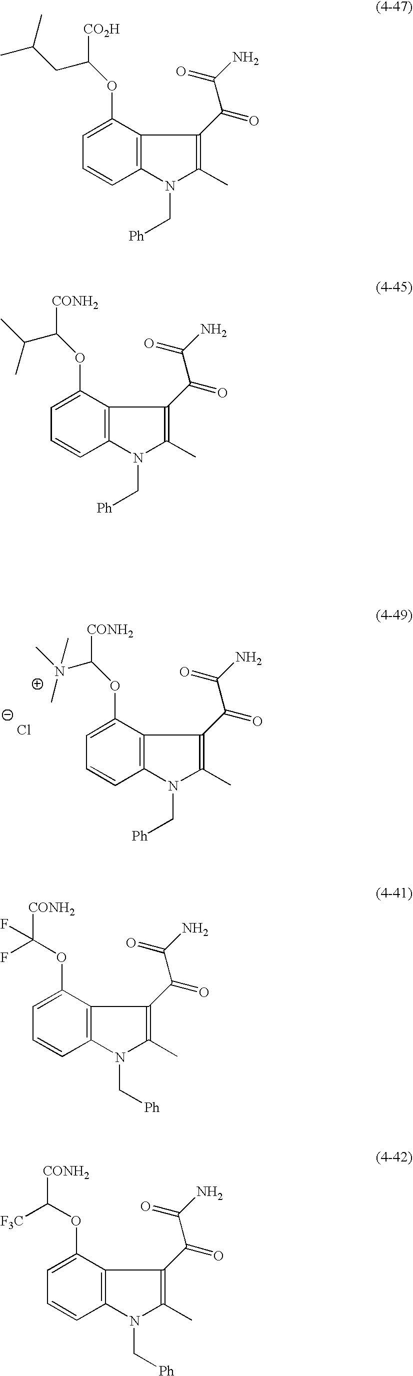

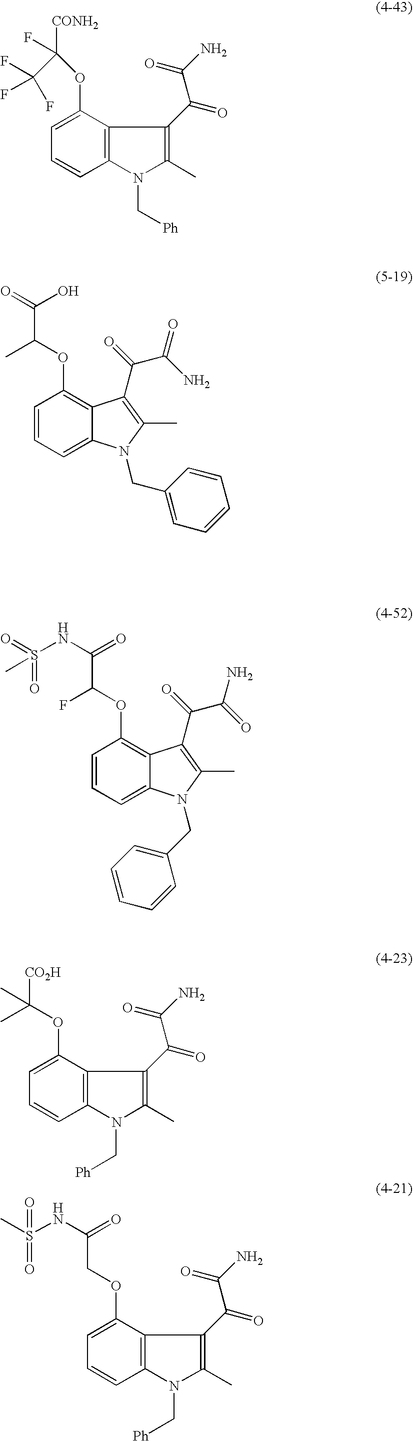

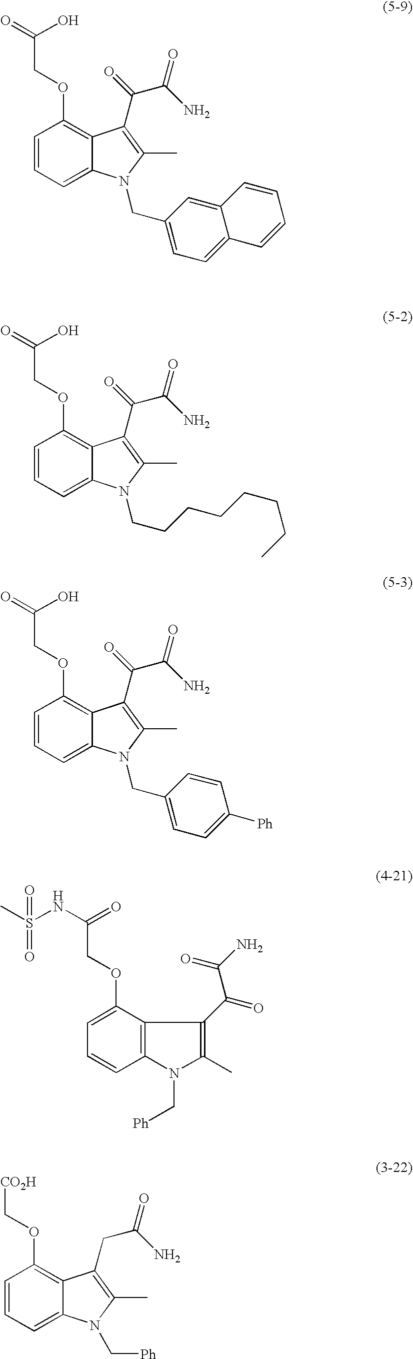

Several schemes are described hereinafter to more fully describe the lumen-localization approach for the above compound based on linking the ILY-4001 indole compound as an inhibition moiety to a polymer moiety. Such schemes are included herein to amplify the discussion of the invention; these schemes are not limiting on the invention, and in particular, similar schemes can be employed for other inhibitor moieties.

In one approach, a functionalized inhibitor moiety can be coupled to a multifunctional bridging moiety through a linking moiety, as described in connection with the first aspect of the invention.

In another polymer-based approach, a functionalized inhibitor moiety can be coupled to a preformed functionalized polymer such as a commercial polymer beads or soluble polymers. For example the linker possesses a halide or an amine to react with amine functionalized or an activated carboxylic acid bead.

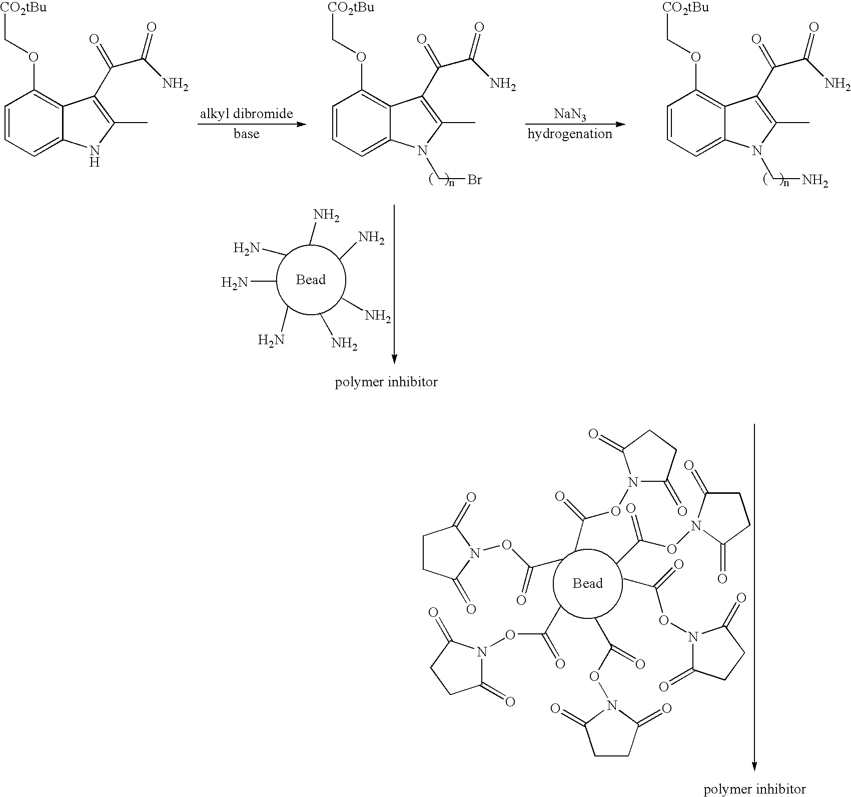

In an additional polymer approach, common monomers are copolymerized with an inhibitor bearing a polymerizable linker. This approach provides random copolymer, or it can provide a block copolymer when living polymerization technique is applied, and alternative, it can provide a crosslinked copolymer when crosslinker is used. With selection of common monomers the material could be hydrophobic, hydrophilic, or their combinations. The inhibitor can be synthesized thru alkylation of indole N1 position as shown in the following scheme:

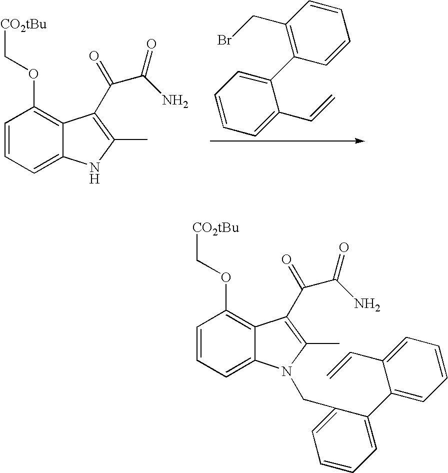

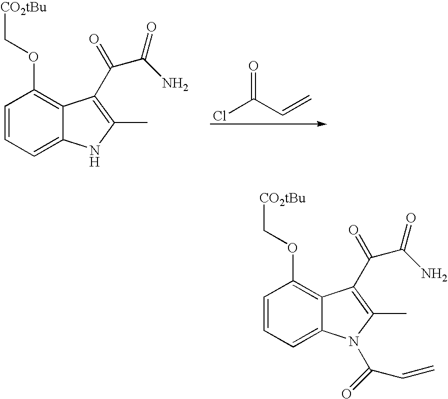

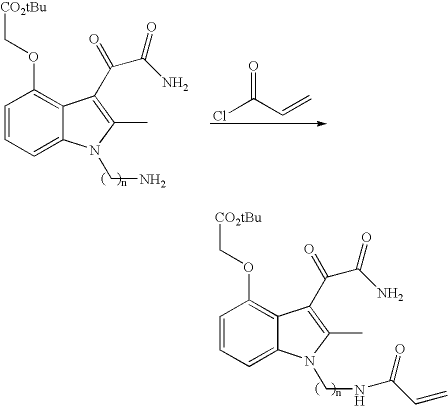

In a further polymer approach, control free radical polymerization can be used to achieve a variety of polymer architectures.

In a first scheme within this third approach, polymer-tailored inhibitors can be prepared. The phospholipase inhibiting moiety bearing a free radical control agent can be synthesized by N1 alkylation with eg. 2-chloro-propionyl chloride or further derivatized to thiourathane. Atom transfer radical polymerization (ATRP) or Reversible addition-fragmentation chain transfer polymerization (RAFT) can be employed to control the chain length of polymer by the ratio of monomers and control agent. The chain end group can be removed by reduction or reserved for dimerization.

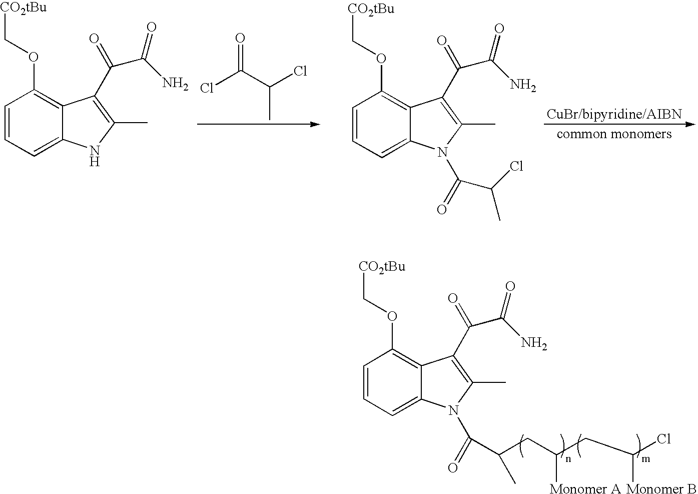

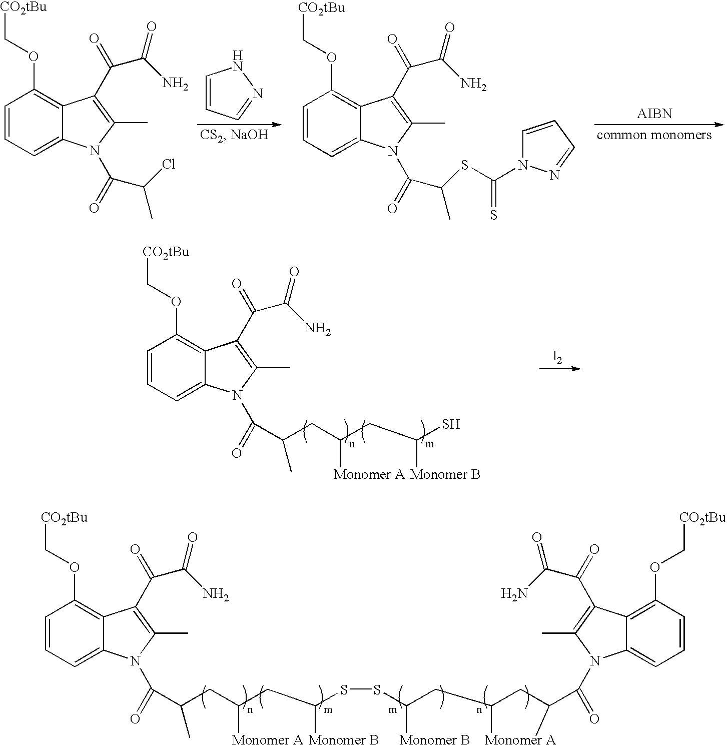

In a second scheme within this third approach, an alternative approach to a short chain inhibitor dimer can be achieved by the route outlined below. Commercial available alkyl dibromide is used as the linker with bromide or thiol end functional group. Then two inhibitor can be jointed by a amine, sulfide, or a disulfide bond. Other jointing functional group also can be applied after derivatization of bromide linker.

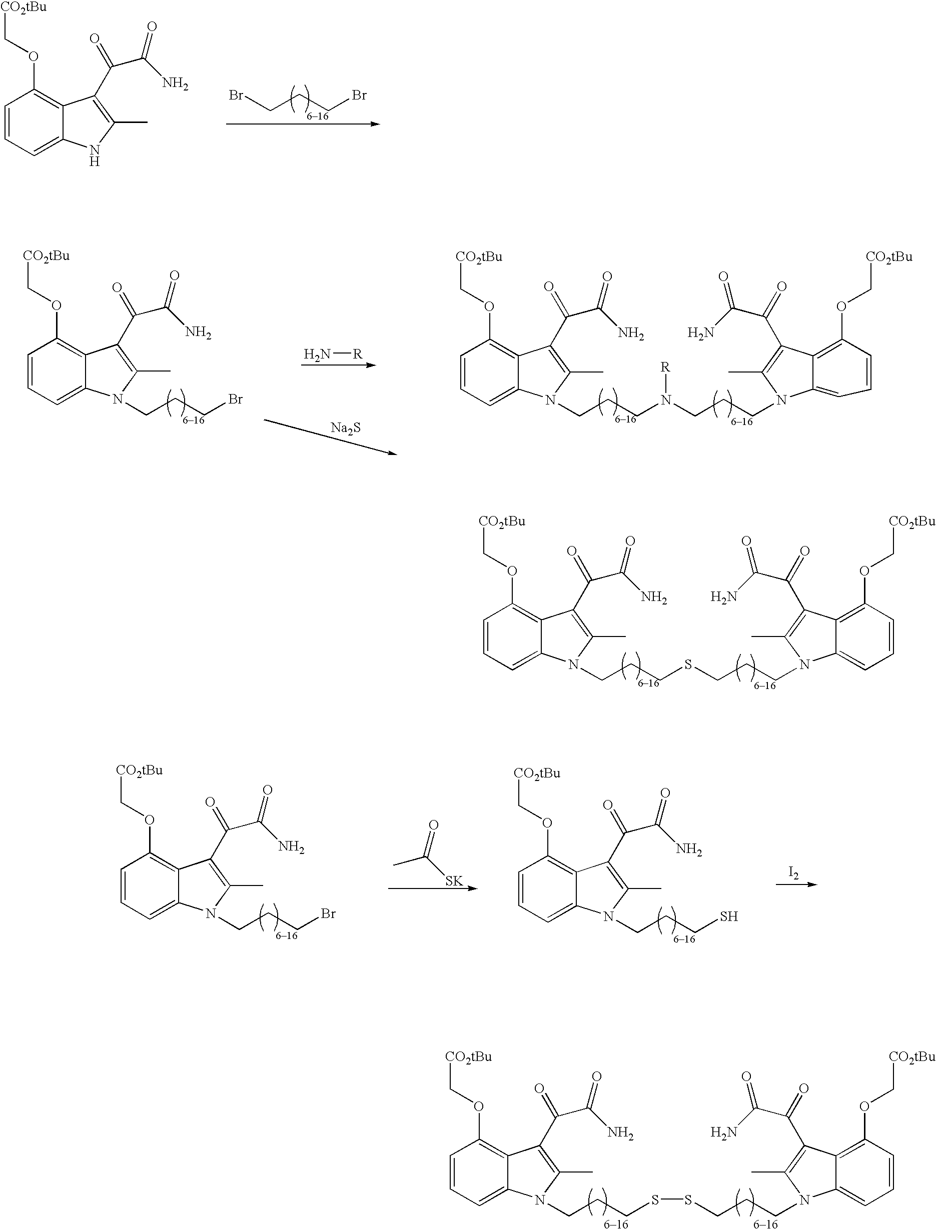

In a third scheme within this third approach involving free-radical polymerization a phospholipase inhibitor-tailored star copolymer can be prepared as follows. The polymer-tailored inhibitor from the first or second schemes within this third approach can be further polymerized with monomers and crosslinker to achieved star copolymer architecture with inhibitor at the chain ends, as shown below:

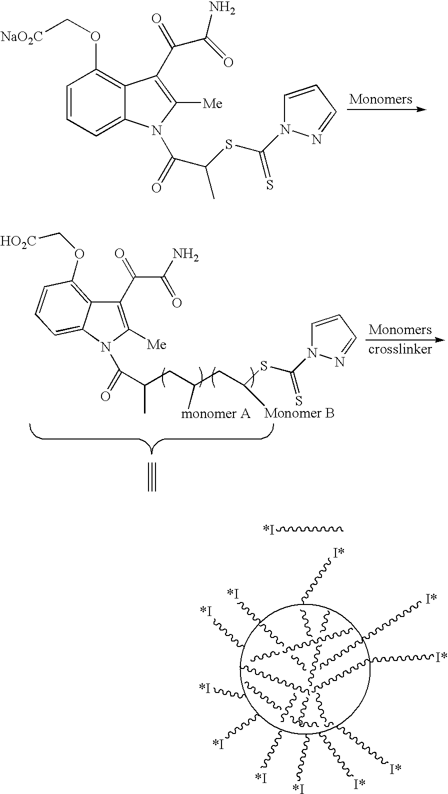

In a fourth scheme within this third approach involving free-radical polymerization, a hyperbranched copolymer can be formed as follows. Copolymerization of control-agent-linked phospholipase inhibitor, AB₂ type monomer, and common monomers provides a hyperbranched copolymer with inhibitor at the chain end as shown below.

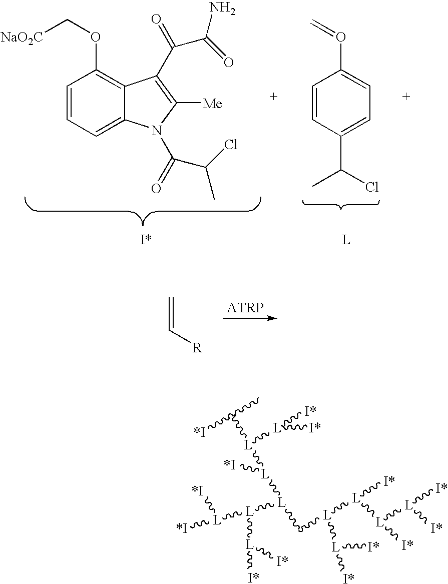

Other art-known phospholipase A2 inhibitors are based on indole compounds or indole-related compounds. (See, for example a summary as shown in co-owned PCT Application No. US/2005/015416 entitled “Treatment of Diet-Related Conditions Using Phospholipase-A2 Inhibitors Comprising Indoles and Related Compounds” filed on May 3, 2005 by Buysse et al.), incorporated herein by reference.

Other art-know phospholipase A2 inhibitors (in addition to the indole and indole-related compounds) are also useful as phospholipase inhibiting moieties of the present invention, and can include the following classes: Alkynoylbenzoic, -Thiophenecarboxylic, -Furancarboxylic, and -Pyridinecarboxylic acids (e.g. see U.S. Pat. No. 5,086,067); Amide carboxylate derivatives (e.g. see WO9108737); Aminoacid esters and amide derivatives (e.g. see WO2002008189); Aminotetrazoles (e.g. see U.S. Pat. No. 5,968,963); Aryoxyacle thiazoles (e.g. see WO00034254); Azetidinones (e.g. see WO9702242); Benzenesulfonic acid derivatives (e.g. see U.S. Pat. No. 5,470,882); Benzoic acid derivatives (e.g. see JP08325154); Benzothiaphenes (e.g. see WO02000641); Benzyl alcohols (e.g. see U.S. Pat. No. 5,124,334); Benzyl phenyl pyrimidines (e.g. see WO00027824); Benzylamines (e.g. see U.S. Pat. No. 5,039,706); Cinammic acid compounds (e.g. see JP07252187); Cinnamic acid derivatives (e.g. see U.S. Pat. No. 5,578,639); Cyclohepta-indoles (e.g. see WO03016277); Ethaneamine-benzenes; Imidazolidinones, Thiazoldinones and Pyrrolidinones (e.g. see WO03031414); Indole glyoxamides (e.g. see U.S. Pat. No. 5,654,326); Indole glyoxamides (e.g. see WO9956752); Indoles (e.g. see U.S. Pat. No. 6,630,496 and WO9943672; Indoly (e.g. see WO003048122); Indoly containing sulfonamides; N-cyl-N-cinnamoylethylenediamine derivatives (e.g. see WO9603371); Naphyl acateamides (e.g. see EP77927); N-substituted glycines (e.g. see U.S. Pat. No. 5,298,652); Phosopholipid analogs (e.g. see U.S. Pat. No. 5,144,045 and U.S. Pat. No. 6,495,596); piperazines (e.g. see WO03048139); Pyridones and Pyrimidones (e.g. see WO03086400); 6-carbamoylpicolinic acid derivatives (e.g. see JP07224038); Steroids and their cyclic hydrocarbon analogs with amino-containing sidechains (e.g. see WO8702367); Trifluorobutanones (e.g. see U.S. Pat. No. 6,350,892 and US2002068722); Abietic derivatives (e.g. see U.S. Pat. No. 4,948,813); Benzyl phosphinate esters (e.g. see U.S. Pat. No. 5,504,073); each of which is incorporated herein by reference.

Specific examples of phospholipase inhibiting moieties of some of these PL A₂ inhibitor classes are provided in Table 1 below, along with IC50 values corresponding thereto:

TABLE 1
Examples of phospholipase inhibiting moiety from a PL A2 inhibitor
class

| Example of phospholipase inhibiting moiety from a PL A₂ inhibitor class | IC50 |
|---|---|
| Alkynoylbenzoic, -Thiophenecarboxylic, -Furancarboxylic, and | μM range |
| Pyridinecarboxylic acids | |
| 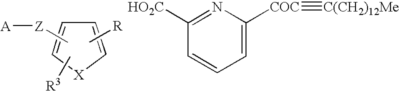 | |
| Amide carboxylate derivatives | sub μM range |
| 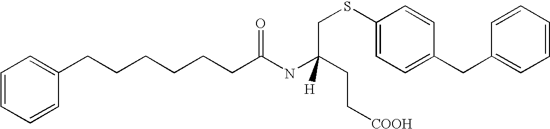 | |
| Aminoacid esters and amide derivatives | about 2.5 μM |
| Aminotetrazoles | μM range |
| 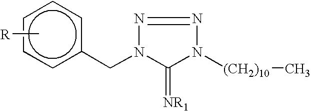 | |
| Aryoxyacyl thiazoles | |
| 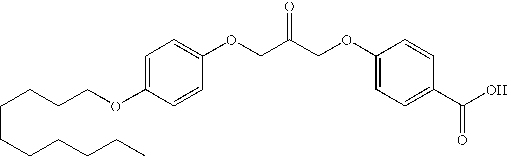 | |
| Azetidinone | |
| 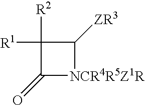 | |
| 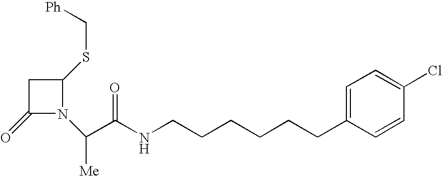 | |
| Benzenesulfonic acid derivatives | |
| 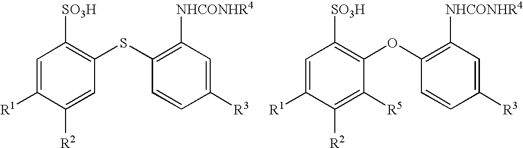 | |
| Benzoic acid derivatives | μM range |
| Benzothiaphenes | about 1.4 μM |
| Benzyl alcohols | about 10 μM |
| 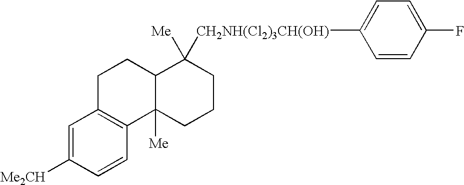 | |
| Benzyl phenyl pyrimidines | |
| Benzylamines | μM range |
| 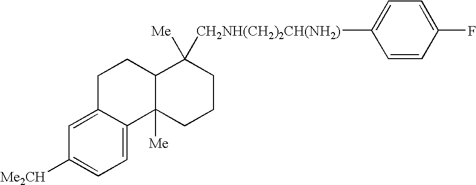 | |
| Cinammic acid compounds | about 70 nM |
| Cinnamic acid derivatives | μM range |
| 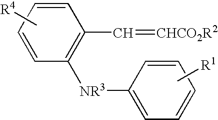 | |
| Cyclohepta-indoles, e.g., preclinical candidate LY-311727 | sub μM range |
| 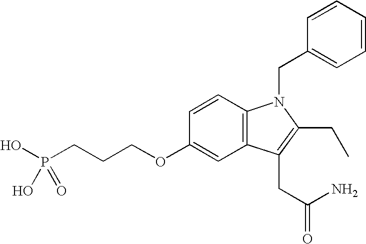 | |
| Ethaneamine-benzenes | μM range |
| 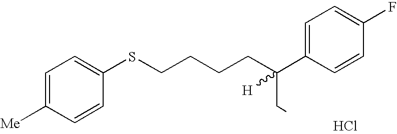 | |
| Imidazolidinones, thiazolidinones and pyrrolidinones | |
| Indole glyoxamides | |
| 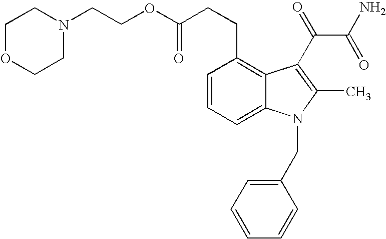 | |
| Indoles | about 0.08 μM to |
| | about 50 μM |
| Indoly containing sulfonamides, e.g., preclinical candidate: PLA-725/PLA - | |
| 902 | |
| 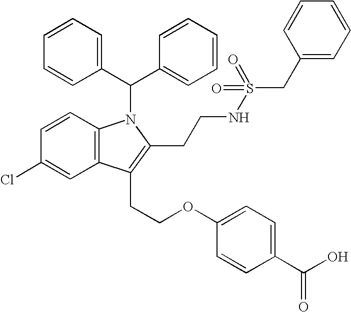 | |
| N-acyl-N-cinnamoylethylenediamine derivatives | about 7 μg/mL |
| 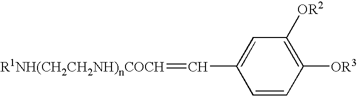 | |
| Naphyl acetamides | about 0.87 nμM |
| 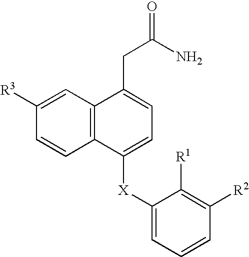 | |
| N-substituted glycines | μM range |
| 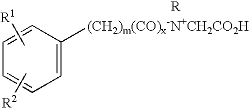 | |
| Phosopholipid analogs | μM range |
| 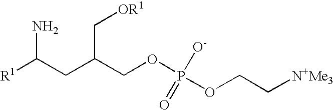 | |
| Piperazines | μM range |
| Pyridones and Pyrimidones, e.g., compound GB-480848 GSK/HGS | nM or subnM range |
| 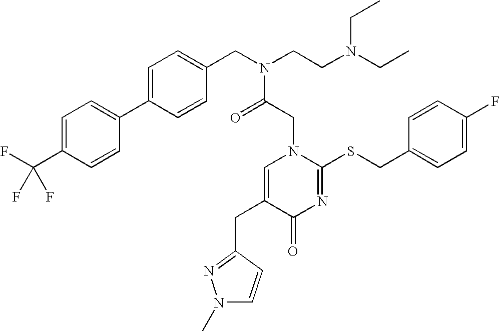 | |
| 6-carbamoy | μM range |
| 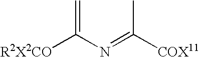 | |
| Steroids and their cyclic hydrocarbon analogs with amino-containing | sub μM range |
| sidechains | |
| 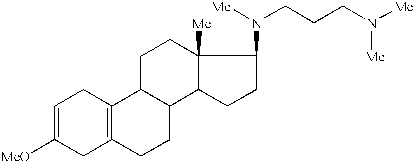 | |
| Trifluorobutanones | about 1 μM to about |
| 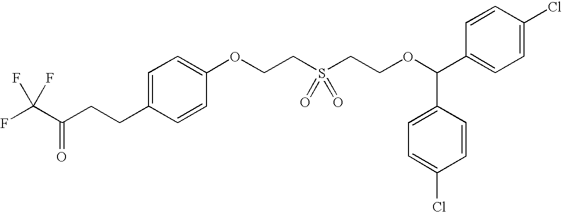 | 50 μM |
| Abietic derivatives | μM range |
| 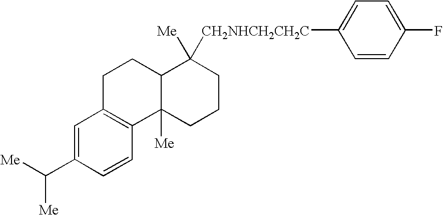 | |
| Benzyl phosphinate esters | μM range |
| 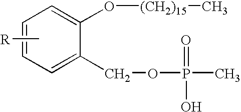 | |
| 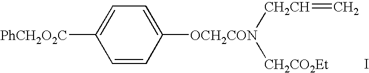 | μM range |

Phospholipase inhibiting moieties useful in some phospholipase inhibitors of the present invention also include natural products, such as Manoalide, a marine product extracted from the sponge Luffariella variabilis, as well as compounds related thereto, illustrated along with the structure of Manoalide below:

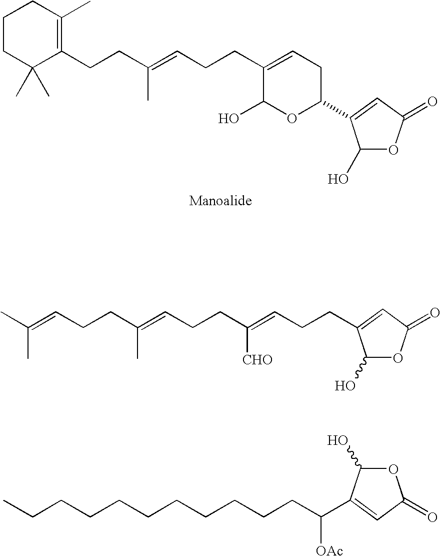

Any of these compounds can be used as a phospholipase inhibiting moiety of the non-absorbed inhibitors in some embodiments of the present invention. As described in more derail above, such moieties may have particular mass, charge and/or other physical parameters to hinder (net) absorption through a gastrointestinal tract, and/or can be linked to a non-absorbed moiety, e.g., a polymer moiety. Furthermore, the invention is not limited to the compositions disclosed herein. Other compositions useful in the present invention would be apparent to one of skill in the art, based on the teachings presented herein, and are also contemplated as within the scope of the invention.

The point of attachment of a phospholipase inhibiting moiety to a non-absorbed moiety, e.g., a polymer moiety, can be selected so as not to interfere with the inhibitory action of the phospholipase inhibiting moiety, e.g., its ability to blunt or reduce the catalytic activity of PL A₂. For instance when a phospholipid analog is used as Z, minimal loss of activity can be achieved by attaching the linking moiety to the hydrophobic group of the phospholipid analog (e.g., its long chain alkyl group) rather than, for example, to its polar head group. Without being limited to a particular hypothesis, phospholipid analogs can inhibit PL A₂ by competing with phospholipid substrates for the catalytic site, which recognizes the polar head group rather than the hydrophobic group of the phospholipid substrate or phospholipid analog. Thus, attachment to the weakly-recognized hydrophobic group can minimize interference with enzyme inhibitory activity of the phospholipid analog. Those of skill in the art will recognize other suitable attachment points for other art-known phospholipase inhibiting moieties.

For example, suitable points of attachment can be identified by available structural information. A co-crystal structure of a phospholipase inhibiting moiety bound to a phospholipase allows one to select one or more sites where attachment of a linking moiety would not preclude the interaction between the phospholipase inhibiting moiety and its target. For instance, preferred points of attachment of phospholipase inhibiting moieties selected from various classes of art-known phospholipase inhibitors are indicated with arrows below:

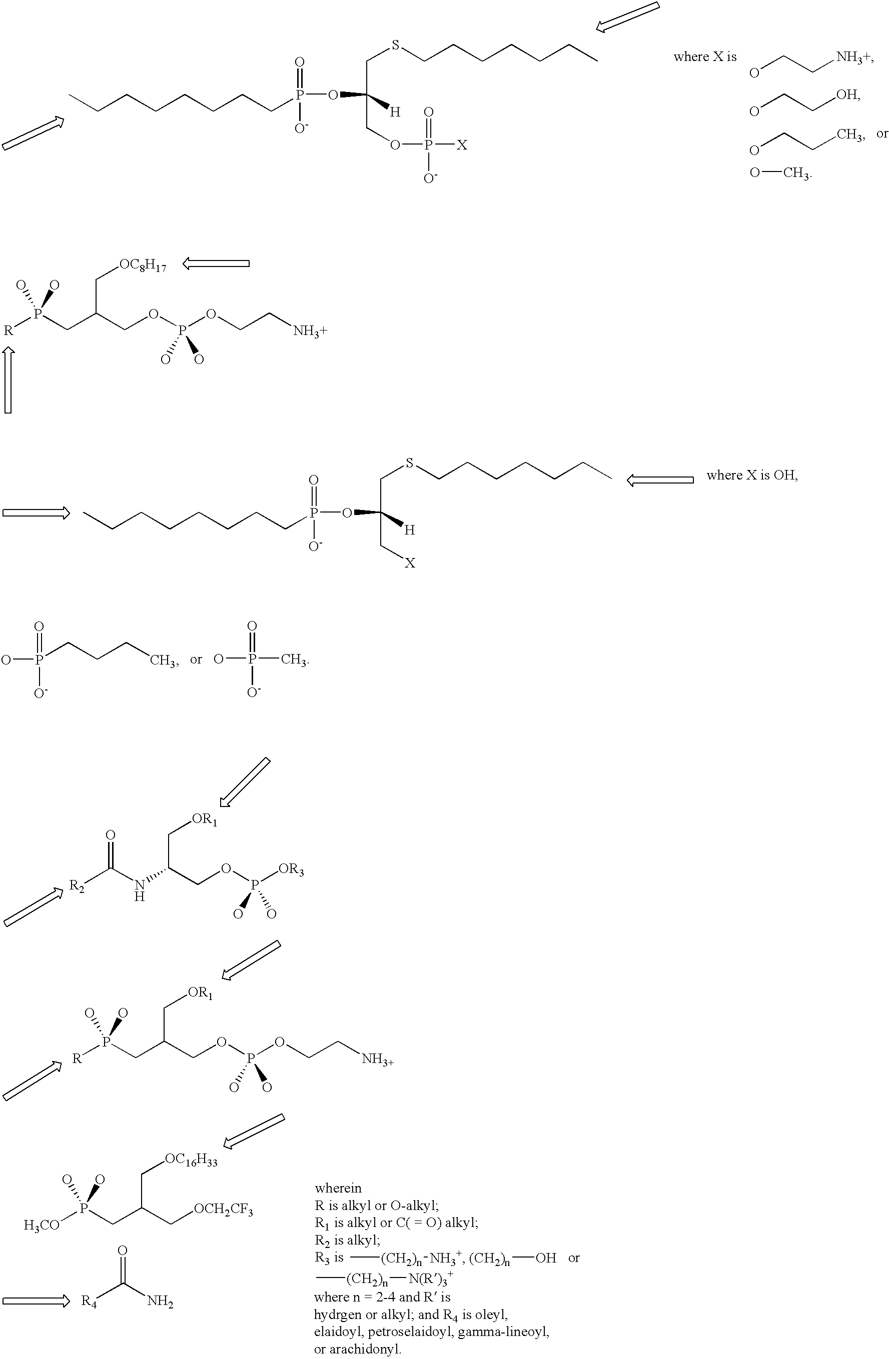

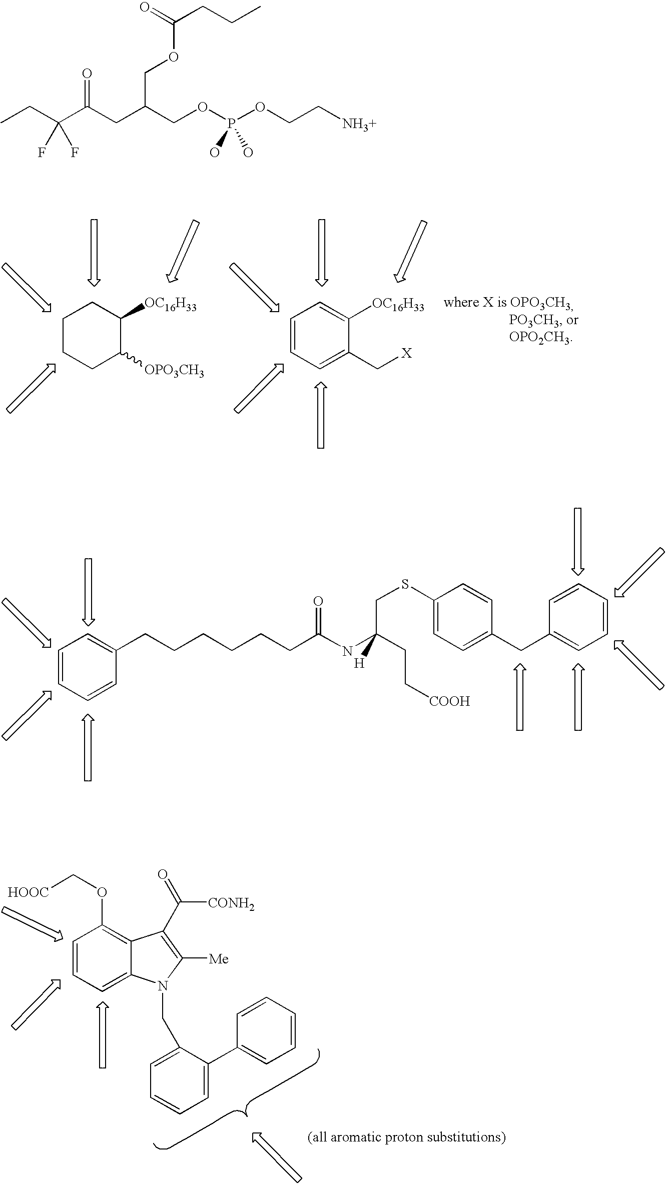

Further, evaluation of binding of a phospholipase inhibitor to a phospholipase by nuclear magnetic resonance permits identification of sites non-essential for such binding interaction. Additionally, one of skill in the art can use available structure-activity relationship (SAR) for phospholipase inhibitors that suggest positions where structural variations are allowed. A library of candidate phospholipase inhibitors can be designed to feature different points of attachment of the phospholipase inhibiting moiety, e.g., chosen based on information described above as well as randomly, so as to present the phospholipase inhibiting moiety in multiple distinct orientations. Candidates can be evaluated for phospholipase inhibiting activity, as discussed in more detail below, to obtain phospholipase inhibitors with suitable attachment points of the phospholipase inhibiting moiety to the polymer moiety or other non-absorbed moiety.

In a third general embodiment, a phospholipase inhibitor or moiety can comprises a small organic molecule. As noted above in connection with the inhibitor moiety of the second general embodiment, a small molecule inhibiting moiety that is lumen-localized can comprise a moiety derived from a substituted organic compound having a fused five-member ring and six-member ring, and preferably a fused five-member ring and six-member ring having one or more heteroatoms (e.g., nitrogen, oxygen) substituted within the ring structure of the five-member ring, within the ring structure of the six-member ring, or within the ring structure of each of the five-member and six-member rings. In each case the inhibiting moiety can comprise substituent groups effective for imparting phospholipase inhibiting functionality to the moiety. Reference is made to the previous discussion above with respect to preferred compounds having fused five-member and six-member rings.

In preferred embodiments, a small molecule phospholipase inhibitor can comprise an indole, such as a substituted indole. Reference is made to the previous discussion above with respect to preferred indole-based compounds.

One small molecule organic compound, ILY-4001, which is represented by the structure: 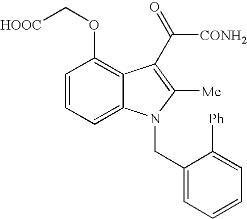
was synthesized (See for example, Example 1A) and evaluated for bioavailability (See, for example, Example 1B). Bioavailability can be reduced (reciprocally, lumen-localization can be improved) according to this third general embodiment of the invention, for example, by charge-modifying strategies applied to this indole moiety to a polymer. (See, for example, Example 1C).

With respect to chemistry for charge modification, general chemistry to indole derivatives is known in literature for example: J. Med. Chem. 1996, 39, 5119-5136; J. Med. Chem. 1996, 39, 5137-5159; J. Med. Chem. 1996, 39, 5159-5175. Chemistry approaches to increase charge moiety on indole derivatives for non-absorbability includes modification of indole C4′, C5, C6, C7, and N1 positions (FIG. 5) with polar groups such as carboxylic, sulfonate, sulfate, phosphonate, phosphate, amine, etc. as an example indole C5 modification uses the commercial available 4-hydroxy indole as a starting material. After selective mild base alkylation on 4-hydroxy position with allyl bromide the 2-phenyl benzyl group is installed at N position using sodium hydride as a base. The standard glyoxamidation is then followed. The subsequent Claisen rearrangement and alkylation of tert-butyl protected acetate give the intermediate with C5 allyl substitution for further polar group installation. 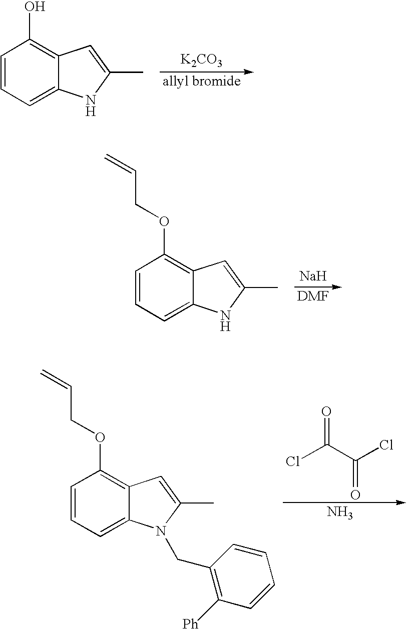 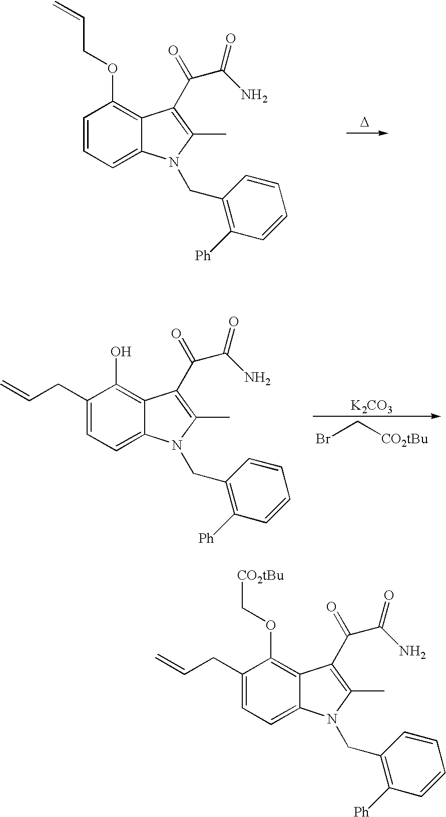
The C5 allyl intermediate is versatile in the sense that not only provides an access to a variety of polar groups but also can modulate length of the group for the SAR study. For example in Pathway A, the target molecule can be obtained via olefin isomerization, ozonolysis, and followed by oxidation to give C5 formic acid derivative. In Pathway B, the allyl intermediate is converted to the corresponding diol by dihydroxylation, then followed by periodate cleavage to afford the aldehyde. Further oxidation of the aldehyde to give acetic acid derivative, or reduction of aldehyde to the corresponding hydroxyl intermediate for further transformation to amine, sulfonate, and phosphonate. In pathway C, the propionic acid derivative can be obtained via hydroboration of olefin and following by oxidation of the corresponding alcohol. In pathway D, the allyl intermediate could simply undergo aminohydroxylation to afford the target. 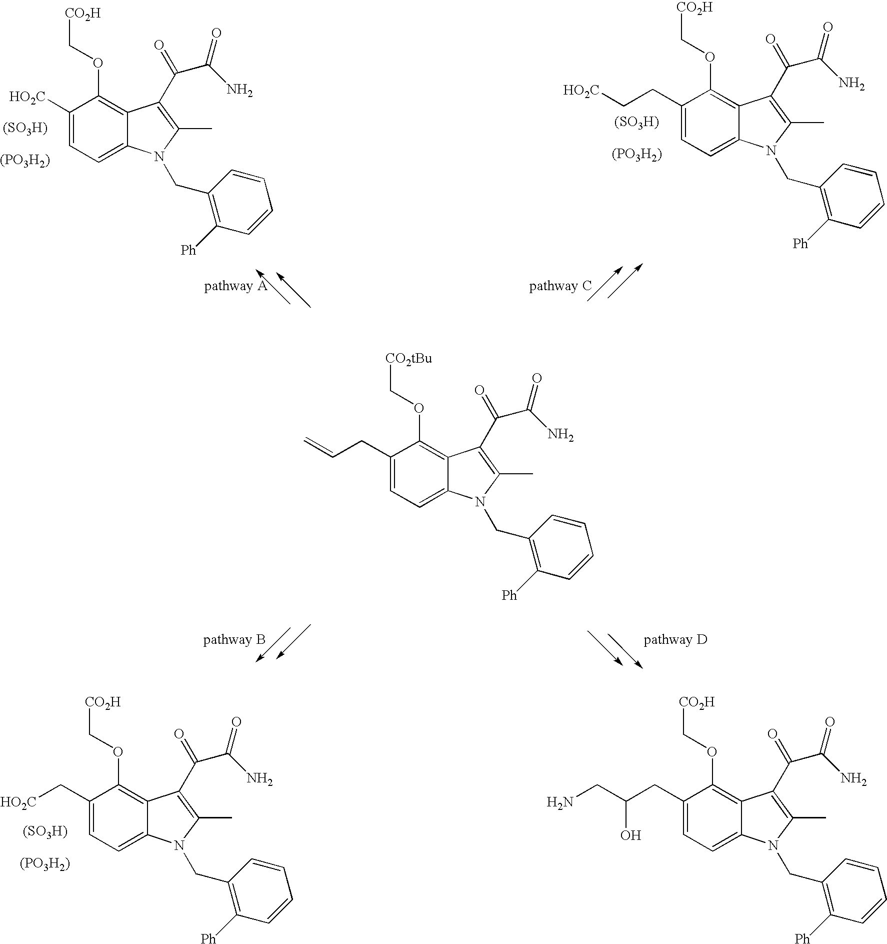
Localization in the Gastrointestinal Lumen Via Efflux

In some embodiments a phospholipase inhibitor is constructed to hinder its (net) absorption through a gastrointestinal mucosa and/or comprises a phospholipase inhibiting moiety linked, coupled or otherwise attached to a non-absorbed moiety as described above. In some embodiments, the phospholipase inhibitor is localized in a gastrointestinal lumen due to efflux. In some embodiments, the inhibitor is effluxed from a gastrointestinal mucosal cell, for example, an intestinal and/or a colonic enterocyte, upon entry into the cell, creating the net effect of non-absorption. Any art-known phospholipase inhibitor and/or any phospholipase inhibiting moiety described and/or contemplated herein can be used in these embodiments. For example, any art known PL A₂ inhibitors provided in Table 1 can be used. These and other art-known phospholipase inhibitors and/or any phospholipase inhibiting moiety disclosed and/or contemplated herein can be constructed to be effluxed back into a gastrointestinal lumen upon movement therefrom.

In some efflux embodiments, the phospholipase inhibitor remains localized in the gastrointestinal lumen even though it may be absorbed by a gastrointestinal mucosal cell by active and/or passive transport, or otherwise permeate through the gastrointestinal wall by active and/or passive transport. The phospholipase inhibitor in some embodiments may have one or more hydrophobic and/or lipophilic moieties, tending to allow diffusion across the plasma membrane of a gastrointestinal mucosal cell. However, subsequent passage across the basolateral membrane and into the portal blood circulation can be regulated by a number of physical and molecular considerations, discussed in detail below. For example, a phospholipase inhibitor that enters an intestinal and/or a colonic enterocyte, e.g., an apical enterocyte, can be subsequently effluxed back into the gastrointestinal lumen.

In some embodiments, efflux is achieved by protein and/or glycoprotein transporters located in a gastrointestinal mucosal cell, for example, in an apical enterocyte of the gastrointestinal tract. Protein and/or glycoprotein transporters include, but are not limited to, for example, ATP-binding cassette transport proteins, such as P-glycoproteins including MDR1 (product of ABCB1 locus) and MRP2, located in the epithelial cells of the gut, for example, in the apical enterocytes of the gastrointestinal tract. Such transports may also be referred to pumps.

In some embodiments, for example, a phospholipase inhibitor can be constructed so as to be recognized by a protein and/or glycoprotein transporter that effluxes the inhibitor from the cytoplasm of an enterocyte back into the gastrointestinal lumen. In some embodiments, the phospholipase inhibitor is constructed so as to allow intracellular modification, e.g., via metabolic processes, within the enterocyte to facilitate recognition by a protein and/or glycoprotein transporter, such that the modified inhibitor serves as a target for transport. Motifs that are recognized by protein and/or glycoprotein transporters of the gut epithelium can be determined by one of ordinary skill in the art. For example, recognition motifs for ATP-binding cassette transport proteins, such as P-glycoproteins including MDR1 (product of ABCB1 locus) and MRP2 can be determined. A phospholipase inhibitor of the present invention may comprise a phospholipase inhibiting moiety linked, coupled, or otherwise attached to a recognition motif moiety. “Recognition motif moiety” as used herein refers to a moiety comprising a motif that is recognized by a transporter, or than can be modified to become recognized by a transporter, where the transporter can effect efflux of a composition comprising the recognition motif moiety into the gastrointestinal lumen, including, for example motifs recognized by protein and/or glycoprotein transporters of the gut epithelium such as ATP-binding cassette transport proteins, P-glycoproteins, MDR1, MRP2, and the like. In some embodiments, the recognition motif moiety serves as a target for a transporter of a gut epithelial cell, causing the transporter to drive the phospholipase inhibitor from the inside of the cell back into the gastrointestinal lumen. Lumen localization achieved by efflux can thus hinder or prevent absorption of the phospholipase inhibitor into the blood circulation.

In preferred embodiments, efflux achieves lumen localization of a significant amount, preferably a statistically significant amount, and more preferably essentially all, of the phospholipase inhibitor introduced into the gastrointestinal lumen. That is, essentially all of the phospholipase inhibitor remains in the gastrointestinal lumen by efflux of some, most, and/or essentially all of any inhibitor that moves out of the gastrointestinal lumen. For example, the effect can be such that at least about 90% of phospholipase inhibitor remains in the gastrointestinal lumen, at least about 95%, at least about 98%, preferably at least about 99%, and more preferably at least about 99.5% remains in the gastrointestinal lumen.

In some embodiments, the phospholipase inhibitor comprises one or more additional efflux enhancing moieties. “Efflux enhancing moiety” as used herein refers to a moiety comprising an efflux enhancer that acts to enhance, aid, increase, activate, promote, or otherwise facilitate efflux of the moiety into the gastrointestinal lumen. For example, the phospholipase inhibitor in some embodiments may comprise a moiety that activates expression of a transporter, for example, a transcription factor and/or an enhancer of a gene encoding a transporter. For example, the nuclear receptor, pregnane X, also referred to as the pregnane X receptor (PXR), induces high levels of MDR1 and/or related transporters. (CITE). In some preferred embodiments, the phospholipase inhibitor is coupled, linked and/or otherwise attached to an efflux enhancing moiety that activates PXR, e.g., by contacting and binding to the nuclear receptor. The higher levels of MDR1 and/or related transporters produced can enhance efflux of phospholipase inhibitor that also comprises, for example, a recognition motif for MDR1. Based on the teachings herein, those of ordinary skill in the art will recognize other efflux enhancing moieties that may be used in these aspects of the invention, and which are also contemplated within its scope.

Some embodiments of the present invention involve a combination of non-absorbed and effluxed inhibitors. In such embodiments, lumen localization is achieved by a combination of non-absorption of the phospholipase inhibitor and efflux of some, most, and/or essentially all of any phospholipase inhibitor that moves out of the gastrointestinal lumen.

Lumen-localization can improve the potency of the phospholipase inhibitor, so that the amount of inhibitor administered can be less than the amount administered in the absence of non-absorption and/or efflux. In some embodiments, non-absorption and/or efflux improves the efficacy of the phospholipase inhibitor. In particular, the inhibitor reduces the activity of phospholipase to a greater extent when localized in the lumen by non-absorption and/or efflux. In such embodiments, the amount of phospholipase inhibitor used can be the same as the recommended dosage levels or higher than this dose or lower than the recommended dose. In some embodiments, non-absorption and/or efflux decreases the dose of phospholipase inhibitor used and thus can increase patient compliance and decrease side-effects.

Phospholipase Inhibition by Lumen-Localized Phospholipase Inhibitors

In addition to lumen-localization functionality, the phospholipase inhibitors of the invention should also have an enzyme-inhibiting functionality.

Generally, the term “inhibits” and its grammatical variations are not intended to require a complete inhibition of enzymatic activity. For example, it can refer to a reduction in enzymatic activity by at least about 50%, at least about 75%, preferably by at least about 90%, more preferably at least about 98%, and even more preferably at least about 99% of the activity of the enzyme in the absence of the inhibitor. Most preferably, it refers to a reduction in enzyme activity by an effective amount that is by an amount sufficient to produce a therapeutic and/or a prophylactic benefit in at least one condition being treated. in a subject receiving phospholipase inhibiting treatment, e.g., as disclosed herein. Conversely, the phrase “does not inhibit” and its grammatical variations does not require a complete lack of effect on the enzymatic activity. For example, it refers to situations where there is less than about 20%, less than about 10%, less than about 5%, preferably less than about 2%, and more preferably less than about 1% of reduction in enzyme activity in the presence of the inhibitor. Most preferably, it refers to a minimal reduction in enzyme activity such that a noticeable effect is not observed. Further, the phrase “does not significantly inhibit” and its grammatical variations refers to situations where there is less than about 40%, less than about 30%, less than about 25%, preferably less than about 20%, and more preferably less than about 15% of reduction in enzyme activity in the presence of the inhibitor. Further, the phrase “essentially does not inhibit” and its grammatical variations refers to situations where there is less than about 30%, less than about 25%, less than about 20%, preferably less than about 15%, and more preferably less than about 10% of reduction in enzyme activity in the presence of the inhibitor.

In some embodiments, a phospholipase inhibitor of the present invention acts to inhibit phospholipase such as phospholipase A₂ by hindering access of the enzyme to its phospholipid substrate; in some embodiments it acts by reducing the enzyme's catalytic activity with respect to its substrate; in some embodiments the phospholipase inhibitor acts by a combination of these two approaches.

As discussed above, some gastrointestinal phospholipases, e.g., most PL A₂ enzymes, act on their substrates while physically proximate to (e.g., “docked”) to a lipid-water interface of a lipid aggregate. As such, catalytic activity can depend at least in part on the enzyme having physical access to the outer surface of lipid aggregates in the gastrointestinal lumen. With reference to the schematic, non-limiting representation illustrated in FIG. 1A, for example, a PL A₂ enzyme 10 can interact with a lipid-water interface 22 of a lipid aggregate 20. The catalytic site 12 of the i-face of the enzyme is depicted by a “notch” on the face that interacts with the lipid aggregate 20.

In some embodiments of the present invention, PL A₂ inhibition is achieved by keeping the enzyme off the outer surface of lipid aggregates, thereby hindering access to phospholipid substrates. FIGS. 1B and 1C illustrate two embodiments of non-absorbed polymeric phospholipase inhibitors that can inhibit enzyme activity by hindering access of the enzyme to a phospholipid substrate at a lipid-water interface. Specifically, referring to FIG. 1B, a non-absorbed phospholipase inhibitor 30 consisting essentially of a polymer moiety having hydrophobic end-regions 32 associates with a lipid-water interface 22, and hinders accessibility of the enzyme 10 to the lipid-water interface 22. FIG. 1C illustrates a non-absorbed phospholipase inhibitor 30 consisting essentially of a polymer interacting with the phospholipase enzyme 10, and hindering accessibility of the enzyme 10 to the lipid-water interface 22. The non-absorbed phospholipase inhibitor 30, consisting essentially of polymer having hydrophobic end-regions 32, can associate with both the phospholipase enzyme 10 and a lipid-water interface 22, as illustrated in FIG. 1D.

A non-absorbed inhibitor that acts by hindering access need not directly interfere with the catalytic site of the enzyme, for example, it need not recognize and/or bind to the enzyme's catalytic site or to any other specific site on the enzyme, such as an allosteric site. Rather, in some embodiments, a non-absorbed phospholipase inhibitor of the present invention may prevent or hinder physical adsorption of the enzyme at a lipid-water interface of one or more types of lipid aggregates found in the gastrointestinal lumen. Examples of a “lipid-water interface” include the outer surface of a lipid aggregate found in the gastrointestinal lumen, including, for example, a fat globule, an emulsion droplet, a vesicle, a mixed micelle, and/or a disk, any one of which may contain triglycerides, fatty acids, bile acids, phospholipids, phosphatidylcholine, lysophospholipids, lysophosphatidylcholine, cholesterol, cholesterol esters, other amphiphiles and/or other diet metabolites.

In preferred embodiments, the inhibitor comprises a polymer moiety capable of interacting with either a phospholipase and/or the lipid-water interface of a lipid aggregate. FIG. 1B illustrates an example where the inhibitor 30 interacts with a lipid-water interface 22 such that it becomes physically complexed, coupled, bound, attached, or otherwise adsorbed to the lipid-water interface 22. The inhibitor 30 can interact with the interface 22 through any bonding interaction, including, for example, covalent, ionic, metallic, hydrogen, hydrophobic, and/or van der Waals bonds, preferably hydrophobic an/or ionic bonds. In the example of FIG. 1B inhibitor interaction with a lipid-water interface 22 is facilitated by hydrophobic bonds. In this depicted embodiment, the inhibitor has two end-regions 32 each of which bears a hydrophopic moiety (depicted by solid rectangles), e.g., phospholipid analogs, that become embedded in the lipid layer via hydrophobic interactions between the moieties of the inhibitor 30 and the hydrophobic chains of the bilayer.

FIG. 1C illustrates an example where the inhibitor 30 interacts with a phospholipase enzyme 10, e.g. PL A₂. In some embodiments, the phospholipase inhibitor 30 comprises a moiety that becomes physically complexed, coupled, bound, attached, or otherwise adsorbed to the enzyme 10 so as to hinder its interaction with a lipid aggregate 20. The inhibitor 30 can be described as scavenging the enzyme in solution to create a complex with it. In some embodiments, the enzyme 10 interacting with the inhibitor 30 is sterically hindered from access to its phospholipid substrate at a lipid-water interface 22, for example, because its approach to the interface 22 is physically hindered.

In some embodiments, the inhibitor comprises a polymer moiety that can be soluble or insoluble under the physiological conditions of the gastrointestinal lumen, and may exist, for example, as dispersed micelles or particles, such as colloidal particles or (insoluble) macroscopic beads, as described in detail above. With reference to FIG. 2, for example, phospholipase inhibitors 30, including both soluble and insoluble inhibitors 30, can comprising polymer moieties covalently linked to phospholipase inhibiting moieties (represented schematically by “I*”). The phospholipase inhibitors 30 can interact with the phospholipase-A₂ 10 in a gastrointestinal fluid, for example, in the vicinity of gastrointestinal lipid vesicles.

Referring now to FIGS. 3A through 3B, for example, the inhibitor 30 comprises a polymer moiety covalently linked to a single inhibiting moiety (represented schematically by I*) as a singlet embodiment or to two inhibiting moieties as a dimer embodiment (in each case as described above). In FIG. 3A, the phospholipase inhibitor 30 comprises a hydrophobic polymer moiety, adapted such that the inhibitor 30 associates with a lipid-water interface 22 of a lipid vesicle 20 (shown with the hydrophobic polymer moiety being substantially integral with the lipid bilayer). In FIG. 3B, the phospholipase inhibitor 30 comprises a polymer moiety having a first hydrophobic block and a second hydrophilic block with the second hydrophilic block being proximal to the phospholipase inhibiting moiety, and adapted such that the inhibitor 30 associates with a lipid-water interface 22 of a lipid vesicle 20 (shown with the hydrophobic block being substantially integral with the lipid bilayer and with the hydrophilic block being substantially associated within the aqueous phase surrounding the lipid bilayer). Referring to FIG. 3C, the phospholipase inhibitor 30 comprises a hydrophobic polymer moiety covalently linked to two inhibiting moieties, and adapted such that the inhibitor 30 associates with a lipid-water interface of a lipid vesicle 20 (shown with the hydrophobic polymer moiety being substantially integral with and looped through the lipid bilayer. These embodiments allow for interaction between the inhibiting moiety and phospholipase-A₂ substantially proximate to the vesicle surface.

Generally, in any aspect or embodiment of the invention requiring a polymer moiety, the polymer moiety of the inhibitor can be shaped in various formats, preferably designed to favor the formation of a complex with a phospholipase, e.g., a complex with PL A₂. For instance, the polymer moiety may comprise a macromolecular scaffold designed to interact with the i-face of PL A₂. As discussed above, the structural features of the i-face are such that the aperture of the slot forming the catalytic site is normal to the i-face plane. The aperture is surrounded by a first crown of hydrophobic residues (mainly leucine and isoleucine residues), which itself is contained in a ring of cationic residues, (including lysine and arginine residues). The polymer moiety may be designed as a macromolecular scaffold comprising a plurality of anionic moieties (e.g., arranged so as to bind to the cationic ring) and/or a plurality of hydrophobic residues (e.g., arranged so as to bind to the hydrophobic crown). In such embodiments, the inhibitor becomes positioned over the catalytic site bearing face of a phospholipase and hinders access to the catalytic site as a “lid” or “cap” blocks access to an aperture.

As described above, the inhibitor can comprises a non-absorbed oligomer or polymer moiety and a phospholipase inhibiting moiety. The phospholipase inhibiting moiety may be coupled, linked or otherwise attached to the non-absorbed moiety. In one embodiment, the inhibiting moiety may be linked, for example, to a polymer moiety that interacts with a lipid-water interface and/or a polymer moiety that interacts with phospholipase. In the latter case, the phospholipase inhibiting moiety may further aid the interaction of the polymer moiety with the phospholipase, e.g., with the i-face of PL A₂.

In some embodiments, for example, a PL A₂ inhibiting moiety is linked, coupled or otherwise attached is coupled to a macromolecular scaffold of a polymer moiety where the PL A₂ inhibiting moiety interacts with the catalytic site of PL A₂ while the macromolecular scaffold interacts with the i-face surrounding the catalytic site. Where the phospholipase inhibiting moiety comprises a phospholipid analog or a transition state analog, the phospholipase inhibiting moiety is preferably coupled via its hydrophobic group, leaving the polar head group of the inhibiting moiety available for binding to the catalytic site, e.g., through the His-calcium-Asp triad discussed above.

Some embodiments comprising a phospholipase inhibiting moiety coupled to a polymer moiety that interacts with a phospholipase comprise a plurality of anionic moieties (e.g., arranged so as to bind to a cationic ring) linked to a spacer moiety (e.g., arranged so as to overlay a hydrophobic crown), which converge on a central or focal point bearing the phospholipase inhibiting moiety. Some such embodiments can be represented by the formula (D) 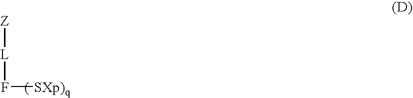
where Z is a phospholipase inhibiting moiety, preferably a PL A₂ inhibiting moiety; L is a linking moiety, e.g., a chemical linker; F is focal point where covalent linkages from a plurality of segments SXp converge; S is a spacer moiety; X is an anionic moiety, preferably an acidic group, for example, but not limited to, a carboxylate group, a sulfonate group, a sulfate group, a sulfamate group, a phosphoramidate group, a phosphate group, a phosphonate group, a phosphinate group, a gluconate group, and the like; and p and q are each integers, preferably where p equals 1, 2, 3, or 4, and preferably where q equals 2, 3, 4, 5, 6, 7, or 8.

The F—(SXp)q segment can adopt various configurations, preferably configurations that facilitate interaction with the catalytic site bearing face of a phospholipase. In some embodiments, for example, a plurality of spacer moieties radiate from the focal point F, which lies at a center of a macromolecular scaffold of the polymer moiety;

In some preferred embodiments, the spacer moiety S provides a plurality of hydrophobic residues, e.g., arranged so as to bind to the hydrophobic crown of the i-face of PL A₂; in some preferred embodiments, the anionic moieties X are arranged so as to bind to the cationic ring of the i-face of PL A₂. Some embodiments comprise a dendritic macromolecular scaffold with spacer moieties branching and diverging from the focal point F. Examples of some embodiments can be represented by the structures provided below:

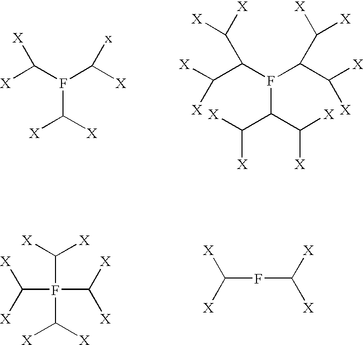

Other examples of dendritic structures useful in the practice of the present invention are known in the art, e.g., see Grayson S. M. et al. Chemical Reviews, 2001, 101: 3819-3867; and Bosman A. W. et al, Chemical Reviews, 1999, 99; 1665-1688, incorporated herein by reference. Additionally, other examples suitable for use in the present invention will be appreciated by those of ordinary skill in the art in light of the disclosures provided herein, and are contemplated as within the scope of this invention.

In some embodiments, the macromolecular scaffold of the polymer moiety can form particles. In such embodiments, a phospholipase inhibiting moiety is preferably coupled to the outer surfaces of such particles. Where the phospholipase inhibiting moiety is a phospholipid analog or transition state analog, the phospholipase inhibiting moiety is preferably linked through its hydrophobic group, as discussed above. The particles so formed may be porous or non-porous, and may be of any shape, such as spherical, elliptical, globular, or irregularly-shaped particles, as discussed in more detail above. The particles can be composed of one or more organic or inorganic polymers moieties, including any of the polymers disclosed herein. In preferred particle embodiments, the particle surface is hydrophobic in nature, carrying acidic groups, X as defined above.

In other embodiments where non-absorbed phospholipase inhibitors comprise a moiety interacting with a specific site on a phospholipase, e.g., the catalytic site of PL A₂, the inhibitor need not prevent access of the enzyme to its substrate, but may act by reducing the enzyme's ability to act on its substrate even if the enzyme approaches and/or becomes “docked” to a lipid-water interface containing the substrate. Such inhibitor embodiments preferably comprise a polymer moiety and one or more phospholipase inhibiting moieties, e.g., an art-known phospholipase inhibitor and/or any phospholipase inhibitor described and/or contemplated herein. Without being bound to a particular hypothesis, for example, such inhibitors can act to reduce phospholipase activity by reversible and/or irreversible inhibition.

Reversible inhibition by a phospholipase inhibitor of the present invention may be competitive (e.g. where the inhibitor binds to the catalytic site of a phospholipase), noncompetitive (e.g., where the inhibitor binds to an allosteric site of a phospholipase to effect an allosteric change), and/or uncompetitive (where the inhibitor binds to a complex between a phospholipase and its substrate). Inhibition may also be irreversible, where the phospholipase inhibitor remains bound, or significantly remains bound, or essentially remains bound to a site on a phospholipase without dissociating, without significantly dissociating, or essentially without dissociating from the enzyme.

As discussed above, PL A₂ enzymes share a conserved active site architecture and a catalytic mechanism involving concerted binding of His and Asp residues to water molecules and a calcium cation. Phospholipid substrate can access the catalytic site by its polar head group through a slot enveloped by hydrophobic and cationic residues. Within the catalytic site, the multi-coordinated calcium ion activates the acyl carbonyl group of the sn-2 position of the phospholipid substrate to bring about hydrolysis. In certain embodiments, PL A₂ inhibiting moieties comprise structures that resemble a phospholipid substrate and/or its transition state.

Without being limited to a particular hypothesis, such moieties can inhibit PL A₂ by competing reversibly with phospholipid substrates for the catalytic site. That is, a structural analog of a phospholipid substrate, preferably, a structural analog of its polar head group and/or a structural analog of a phospholipid substrate transition state can reversibly bind the catalytic site, inhibiting access of the phospholipid substrate. Further, as described in detail above, analog phospholipase inhibiting moieties can be attached to a non-absorbed moiety, e.g., a polymer moiety, at an attachment point that does not interfere with the ability of the analog to bind to the catalytic site, minimizing the inhibitory activity of the analog.

In view of the substantial structure-activity-relationship studies for phospholipase-A2 enzymes, considered together with the significant experimental data demonstrated in Example 5 (including Examples 5A through 5C), a skilled person can appreciate that the observed inhibitive effect of ILY-4001 can be realized in other indole compounds of the invention (having the identical core structure) as well as in indole-related compounds comprising a fused five-membered ring and six-membered ring. In particular, without being bound by theory not expressly recited in the claims, a skilled person can appreciate, with reference to FIG. 6A, for example, that substituents at positions 3 and 4 and 5 of the indole structure can be selected and evaluated to be effective for polar interaction with the enzyme and with calcium ion (associated with the calcium-dependent phospholipase activity). Similarly, a person of skill in the art can appreciate that the substituents at positions 1 and 2 of the indole structure can be selected and evaluated to be relatively hydrophobic. Considered in combination, the polar groups at positions 3, 4 and 5 and the relatively hydrophobic groups at positions 1 and 2 can effectively associate the inhibitor (or inhibiting moiety) with a hydrophilic lipid-water interface (via the hydrophobic regions), and also orient the inhibitor (or inhibiting moiety) such that its polar region can be effectively positioned into the enzyme pocket—with its polar head group directed through a slot enveloped by hydrophobic and cationic residues. Similarly, with reference to FIG. 6B, for example, one can appreciate that corresponding groups on the indole-related compound shown therein can have the same functionality. Specifically, a person of skill in the art can appreciate that substituents at positions R₃, R₄ and R₅ of the indole-related structure can be selected and evaluated to be effective for polar interaction with the enzyme and with calcium ion, and that the substituents at positions R₁ and R₂ of the indole-related structure can be selected and evaluated to be relatively hydrophobic.

Similarly, with reference to FIGS. 6C and 6D, the above-described inverse indole compounds that are mirror-image analogues of the core structure of the corresponding indole of interest, and the above-described reciprocal indole compounds and reciprocal indole-related compounds that are alternative mirror-image analogues of the core structure of the corresponding indole or related compound can be similarly configured with polar substituents and hydrophobic substituents to provide alternative indole structures and alternative indole-related structures within the scope of the invention.

Moreover, a person skilled in the art can evaluate particular inhibitors within the scope of this invention using known assaying and evaluation approaches. For example, the extent of inhibition of the inhibitors of the invention can be evaluated using in-vitro assays (See, for example, Example 1B-1) and/or in-vivo studies (See, for example, Example 10).

Further, in some of these embodiments, the phospholipase inhibitor reduces re-absorption of secreted phospholipase A2 through the gastrointestinal mucosa.

Screening Assays for Identifying Phospholipase Inhibitors

The differential activities of gastrointestinal phospholipases, in particular phospholipase A2, enables the screening for inhibitory compounds that inhibit a particular phospholipase and that can be used with the practice of this invention to selectively treat insulin-related conditions (e.g., diabetes), weight-related conditions (e.g., obesity), cholesterol-related conditions, or a combination thereof.

Certain approaches of the present invention provide a method of making or identifying a phospholipase inhibitor that is localized in a gastrointestinal lumen involving selecting a moiety that inhibits PL A₂ by contacting a candidate moiety with a PL A₂ enzyme or fragment thereof, preferably a fragment containing the catalytic and/or allosteric site of the enzyme, more preferably including the His and Asp residues of the catalytic site; determining whether the candidate moiety interacts with the PL A₂ or fragment thereof; and using the selected candidate moiety as a phospholipase A2 inhibiting moiety of a phospholipase inhibitor that is localized in a gastrointestinal lumen.

Certain other approaches of the present invention provide a method of making or identifying a phospholipase inhibitor that is localized in a gastrointestinal lumen involving selecting a moiety that inhibits PL A₂ by contacting a candidate moiety with a lipid-water interface of a lipid aggregate or fragment thereof; determining whether the candidate moiety interacts with the interface; and using the selected candidate moiety as a phospholipase A2 inhibiting moiety of a phospholipase inhibitor that is localized in a gastrointestinal lumen.

Certain approaches of the present invention provide a method of making or identifying a phospholipase inhibitor that is localized in a gastrointestinal lumen involving selecting a moiety that inhibits PLB by contacting a candidate moiety with a PLB enzyme or fragment thereof; determining whether the candidate moiety interacts with the PLB or fragment thereof; and using the selected candidate moiety as a phospholipase B inhibiting moiety of a phospholipase inhibitor that is localized in a gastrointestinal lumen.

Certain approaches of the present invention provide a method of making or identifying a phospholipase inhibitor that is localized in a gastrointestinal lumen involving selecting a moiety that preferentially inhibits PL A₂ by contacting a candidate moiety with a PL A₂ enzyme or fragment thereof, preferably a fragment containing the catalytic and/or allosteric site of the enzyme, more preferably including the His and Asp residues of the catalytic site and determining whether the candidate moiety interacts with the PL A₂ or fragment thereof; contacting the candidate with a PLB enzyme or fragment thereof and determining whether the candidate interacts with the PLB or fragment thereof; selecting any candidate that interacts with PL A₂ but does not interact with PLB, does not significantly interact with PLB, or essentially does not interact with PLB; and using the selected candidate moiety as a phospholipase A2 inhibiting moiety of a phospholipase inhibitor that is localized in a gastrointestinal lumen.

Certain other approaches of the present invention provide a method of making or identifying a phospholipase inhibitor that is localized in a gastrointestinal lumen involving selecting a moiety that preferentially inhibits PL A₂ by contacting a candidate with a lipid-water interface of a lipid aggregate or fragment thereof and determining whether the candidate moiety interacts with the interface; contacting the candidate moiety with a PLB enzyme or fragment thereof and determining whether the candidate moiety interacts with the PLB or fragment thereof; selecting any candidate moiety that interacts with the lipid-water interface does not interact with PLB, but does not significantly interact with PLB, or essentially does not interact with PLB, and using the selected candidate moiety as a phospholipase A2 inhibiting moiety of a phospholipase inhibitor that is localized in a gastrointestinal lumen.

A lumen-localized phospholipase inhibitor, for example, comprising a phospholipase inhibiting moiety disclosed herein and/or identified by the procedures taught herein, can be used in animal models to demonstrate, for example, suppression of insulin-related conditions (e.g. diabetes) and/or hypercholesterolemia and/or weight-related conditions. A lumen-localized phospholipase inhibitor showing inhibitory activity in a PL A₂ inhibition assay, in about the sub μM range is preferred. More preferably, such inhibitors show non-absorbedness, for example low permeability, in any assays disclosed herein or known in the art. Examples of suitable animal models are described in more detail below.

Non-absorbed and/or effluxed phospholipase inhibitors of the present invention can form the basis of pharmaceutical compositions and kits that find use in methods of treating a subject by administering the composition. Preferably, such compositions modulate the activity of a gastrointestinal phospholipase, for example, reducing the activity of phospholipase A₂ and/or one or more other phospholipases. In some embodiments, the phospholipase inhibitor inhibits phospholipase A₂. In some embodiments, the phospholipase inhibitor inhibits phospholipase A₂ and phospholipase B. In some embodiments, the phospholipase inhibitor inhibits phospholipase A₂ but does not inhibit or does not significantly inhibit or essentially does not inhibit phospholipase B. In some embodiments, the phospholipase inhibitor inhibits phospholipase A₂ but does not inhibit or does not significantly inhibit or essentially does not inhibit other gastrointestinal phospholipases.

Methods of Treating Phospholipase-Related Conditions

The present invention provides methods of treating phospholipase-related conditions where the inhibitor is localized in a gastrointestinal lumen. Preferably, such inhibitors are administered orally, and preferably in a treatment protocol involving administering of PLA2 inhibitor during or shortly after meals.

The term “phospholipase-related condition” as used herein refers to a condition in which modulating the activity and/or re-absorption of a phospholipase, and/or modulating the production and/or effects of one or more products of the phospholipase, is desirable. In preferred embodiments, an inhibitor of the present invention reduces the activity and/or re-absorption of a phospholipase, and/or reduces the production and/or effects of one or more products of the phospholipase. The term “phospholipase A2-related condition” as used herein refers to a condition in which modulating the activity and/or re-absorption of phospholipase A2 is desirable and/or modulating the production and/or effects of one or more products of phospholipase A2 activity is desirable. In preferred embodiments, an inhibitor of the present invention reduces the activity and/or re-absorption of phospholipase A2, and/or reduces the production and/or effects of one or more products of the phospholipase A2. Examples of phospholipase A2-related conditions include, but are not limited to, insulin-related conditions (e.g., diabetes), weight-related conditions (e.g., obesity) and/or cholesterol-related conditions, and any combination thereof.

The present invention provides methods, pharmaceutical compositions, and kits for the treatment of animal subjects. The term “animal subject” as used herein includes humans as well as other mammals. For example, the mammals can be selected from mice, rats, rabbits, guinea pigs, hamsters, cats, dogs, porcine, poultry, bovine and horses, as well as combinations thereof.

The term “treating” as used herein includes achieving a therapeutic benefit and/or a prophylactic benefit. By therapeutic benefit is meant eradication or amelioration of the underlying disorder being treated. For example, in a diabetic patient, therapeutic benefit includes eradication or amelioration of the underlying diabetes. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For example, with respect to diabetes reducing PL A₂ activity can provide therapeutic benefit not only when insulin resistance is corrected, but also when an improvement is observed in the patient with respect to other disorders that accompany diabetes like fatigue, blurred vision, or tingling sensations in the hands or feet. For prophylactic benefit, a phospholipase inhibitor of the present invention may be administered to a patient at risk of developing a phospholipase-related condition, e.g., diabetes, obesity, or hypercholesterolemia, or to a patient reporting one or more of the physiological symptoms of such conditions, even though a diagnosis may not have been made.

The present invention provides compositions comprising a phospholipase inhibitor that is not absorbed through a gastrointestinal mucosa and/or that is localized in a gastrointestinal lumen as a result of efflux from a gastrointestinal mucosal cell. In preferred embodiments, the phospholipase inhibitors of the present invention produce a benefit, including either a prophylactic benefit, a therapeutic benefit, or both, in treating one or more conditions by inhibiting phospholipase activity.

The methods for effectively inhibiting phospholipase described herein can apply to any phospholipase-related condition, that is, to any condition in which modulating the activity and/or re-absorption of a phospholipase, and/or modulating the production and/or effects of one or more products of the phospholipase, is desirable. Preferably, such conditions include phospholipase-A₂-related conditions and/or phospholipase A2-related conditions induced by diet, that is, conditions which are brought on, accelerated, exacerbated, or otherwise influenced by diet. Phospholipase-A₂-related conditions include, but are not limited to, diabetes, weight gain, and cholesterol-related conditions, as well as hyperlipidemia, hypercholesterolemia, cardiovascular disease (such as heart disease and stroke), hypertension, cancer, sleep apnea, osteoarthritis, gallbladder disease, fatty liver disease, diabetes type 2 and other insulin-related conditions. In some embodiments, one or more of these conditions may be produced as a result of consumption of a high fat or Western diet; in some embodiments, one or more of these conditions may be produced as a result of genetic causes, metabolic disorders, environmental factors, behavioral factors, or any combination of these.

Western Diets and Western-Related Diets

Generally, some embodiments of the invention relate to one or more of a high-carbohydrate diet, a high-saccharide diet, a high-fat diet and/or a high-cholesterol diet, in various combinations. Such diets are generally referred to herein as a “high-risk diets” (and can include, for example, Western diets). Such diets can heighten the risk profile of a subject patient for one or more conditions, including an obesity-related condition, an insulin-related condition and/or a cholesterol-related condition. In particular, such high-risk diets can, in some embodiments, include at least a high-carbohydrate diet together with one or more of a high-saccharide diet, a high-fat diet and/or a high-cholesterol diet. A high-risk diet can also include a high-saccharide diet in combination with one or both of a high-fat diet and/or a high-cholesterol diet. A high-risk diet can also comprise a high-fat diet in combination with a high-cholesterol diet. In some embodiments, a high-risk diet can include the combination of a high-carbohydrate diet, a high-saccharide diet and a high-fat diet. In other embodiments, a high-risk diet can include a high-carbohydrate diet, a high-saccharide diet, and a high-cholesterol diet. In other embodiments, a high-risk diet can include a high-carbohydrate diet, a high-fat diet and a high-cholesterol diet. In yet further embodiments, a high-risk diet can include a high-saccharide diet, a high-fat diet and a high-cholesterol diet. In some embodiments, a high-risk diet can include a high-carbohydrate diet, a high-saccharide diet, a high-fat diet and a high-cholesterol diet.

Generally, the diet of a subject can comprise a total caloric content, for example, a total daily caloric content. In some embodiments, the subject diet can be a high-fat diet. In such embodiments, at least about 50% of the total caloric content can come from fat. In other such embodiments, at least about 40%, or at least about 30% or at least about 25%, or at least about 20% of the total caloric content can come from fat. In some embodiments, in which a high-fat diet is combined with one or more of a high-carbohydrate diet, a high-saccharide diet or a high-cholesterol diet, at least about 15% or at least about 10% of the total caloric content can come from fat.

Similarly, in some embodiments, the diet can be a high-carbohydrate diet. In such embodiments, at least about 50% of the total caloric content can come from carbohydrates. In other such embodiments, at least about 40%, or at least about 30% or at least about 25%, or at least about 20% of the total caloric content can come from carbohydrates. In some embodiments, in which a high-carbohydrate diet is combined with one or more of a high-fat diet, a high-saccharide diet or a high-cholesterol diet, at least about 15% or at least about 10% of the total caloric content can come from carbohydrate.

Further, in some embodiments, the diet can be a high-saccharide diet. In embodiments, at least about 50% of the total caloric content can come from saccharides. In other such embodiments, at least about 40%, or at least about 30% or at least about 25%, or at least about 20% of the total caloric content can come from saccharides. In some embodiments, in which a high-saccharide diet is combined with one or more of a high-fat diet, a high-carbohydrate diet or a high-cholesterol diet, at least about 15% or at least about 10% of the total caloric content can come from saccharides.

Similarly, in some embodiments, the diet can be a high-cholesterol diet. In such embodiments, the diet can comprise at least about 1% cholesterol (wt/wt, relative to fat). In other such embodiments, the diet can comprise at least about 0.5% or at least about 0.3% or at least about 0.1%, or at least about 0.07% cholesterol (wt/wt relative to fat). In some embodiments, in which a high-cholesterol diet is combined with one or more of a high-fat diet, a high-carbohydrate diet or a high-saccharide diet, the diet can comprise at least about 0.05% or at least about 0.03% cholesterol (wt/wt, relative to fat).

As an example, a high fat diet can include, for example, diets high in meat, dairy products, and alcohol, as well as possibly including processed food stuffs, red meats, soda, sweets, refined grains, deserts, and high-fat dairy products, for example, where at least about 25% of calories come from fat and at least about 8% come from saturated fat; or at least about 30% of calories come from fat and at least about 10% come from saturated fat; or where at least about 34% of calories came from fat and at least about 12% come from saturated fat; or where at least about 42% of calories come from fat and at least about 15% come from saturated fat; or where at least about 50% of calories come from fat and at least about 20% come from saturated fat. One such high fat diet is a “Western diet” which refers to the diet of industrialized countries, including, for example, a typical American diet, Western European diet, Australian diet, and/or Japanese diet. One particular example of a Western diet comprises at least about 17% fat and at least about 0.1% cholesterol (wt/wt); at least about 21% fat and at least about 0.15% cholesterol (wt/wt); or at least about 25% and at least about 0.2% cholesterol (wt/wt).

Such high-risk diets may include one or more high-risk foodstuffs.

Considered in the context of a foodstuff, generally, some embodiments of the invention relate to one or more of a high-carbohydrate foodstuff, a high-saccharide foodstuff, a high-fat foodstuff and/or a high-cholesterol foodstuff, in various combinations. Such foodstuffs are generally referred to herein as a “high-risk foodstuffs” (including for example Western foodstuffs). Such foodstuffs can heighten the risk profile of a subject patient for one or more conditions, including an obesity-related condition, an insulin-related condition and/or a cholesterol-related condition. In particular, such high-risk foodstuffs can, in some embodiments, include at least a high-carbohydrate foodstuff together with one or more of a high-saccharide foodstuff, a high-fat foodstuff and/or a high-cholesterol foodstuff. A high-risk foodstuff can also include a high-saccharide foodstuff in combination with one or both of a high-fat foodstuff and/or a high-cholesterol foodstuff. A high-risk foodstuff can also comprise a high-fat foodstuff in combination with a high-cholesterol foodstuff. In some embodiments, a high-risk foodstuff can include the combination of a high-carbohydrate foodstuff, a high-saccharide foodstuff and a high-fat foodstuff. In other embodiments, a high-risk foodstuff can include a high-carbohydrate foodstuff, a high-saccharide foodstuff, and a high-cholesterol foodstuff. In other embodiments, a high-risk foodstuff can include a high-carbohydrate foodstuff, a high-fat foodstuff and a high-cholesterol foodstuff. In yet further embodiments, a high-risk foodstuff can include a high-saccharide foodstuff, a high-fat foodstuff and a high-cholesterol foodstuff. In some embodiments, a high-risk foodstuff can include a high-carbohydrate foodstuff, a high-saccharide foodstuff, a high-fat foodstuff and a high-cholesterol foodstuff.

Hence, the food product composition can comprise a foodstuff having a total caloric content. In some embodiments, the food-stuff can be a high-fat foodstuff. In such embodiments, at least about 50% of the total caloric content can come from fat. In other such embodiments, at least about 40%, or at least about 30% or at least about 25%, or at least about 20% of the total caloric content can come from fat. In some embodiments, in which a high-fat foodstuff is combined with one or more of a high-carbohydrate foodstuff, a high-saccharide foodstuff or a high-cholesterol foodstuff, at least about 15% or at least about 10% of the total caloric content can come from fat.

Similarly, in some embodiments, the food-stuff can be a high-carbohydrate foodstuff. In such embodiments, at least about 50% of the total caloric content can come from carbohydrates. In other such embodiments, at least about 40%, or at least about 30% or at least about 25%, or at least about 20% of the total caloric content can come from carbohydrates. In some embodiments, in which a high-carbohydrate foodstuff is combined with one or more of a high-fat foodstuff, a high-saccharide foodstuff or a high-cholesterol foodstuff, at least about 15% or at least about 10% of the total caloric content can come from carbohydrate.

Further, in some embodiments, the food-stuff can be a high-saccharide foodstuff. In such embodiments, at least about 50% of the total caloric content can come from saccharides. In other such embodiments, at least about 40%, or at least about 30% or at least about 25%, or at least about 20% of the total caloric content can come from saccharides. In some embodiments, in which a high-saccharide foodstuff is combined with one or more of a high-fat foodstuff, a high-carbohydrate foodstuff or a high-cholesterol foodstuff, at least about 15% or at least about 10% of the total caloric content can come from saccharides.

Similarly, in some embodiments, the food-stuff can be a high-cholesterol foodstuff. In such embodiments, the food-stuff can comprise at least about 1% cholesterol (wt/wt, relative to fat). In other such embodiments, the foodstuff can comprise at least about 0.5%, or at least about 0.3% or at least about 0.1%, or at least about 0.07% cholesterol (wt/wt relative to fat). In some embodiments, in which a high-cholesterol foodstuff is combined with one or more of a high-fat foodstuff, a high-carbohydrate foodstuff or a high-saccharide foodstuff, the foodstuff can comprise at least about 0.05% or at least about 0.03% cholesterol (wt/wt, relative to fat).

As noted above, the methods of the invention can be used advantageously together with other methods, including for example methods broadly directed to treating insulin-related conditions, weight-related conditions and/or cholesterol-related conditions (including dislipidemia generally) and any combination thereof. Aspects of such conditions are described below.

Treatment of Insulin-Related Conditions

The term “insulin-related disorders” as used herein refers to a condition such as diabetes where the body does not produce and/or does not properly use insulin. Typically, a patient is diagnosed with pre-diabetes or diabetes by using a Fasting Plasma Glucose Test (FPG) and/or an Oral Glucose Tolerance Test (OGTT). In the case of the FPG test, a fasting blood glucose level between about 100 and about 125 mg/dl can indicate pre-diabetes; while a person with a fasting blood glucose level of about 126 mg/dl or higher can indicate diabetes. In the case of the OGTT test, a patient's blood glucose level can be measured after a fast and two hours after drinking a glucose-rich beverage. A two-hour blood glucose level between about 140 and about 199 mg/dl can indicate pre-diabetes; while a two-hour blood glucose level at about 200 mg/dl or higher can indicate diabetes.

In certain embodiments, a lumen localized phospholipase inhibitor of the present invention produces a benefit in treating an insulin-related condition, for example, diabetes, preferably diabetes type 2. For example, such benefits may include, but are not limited to, increasing insulin sensitivity and improving glucose tolerance. Other benefits may include decreasing fasting blood insulin levels, increasing tissue glucose levels and/or increasing insulin-stimulated glucose metabolism.

Without being limited to any particular hypothesis, these benefits may result from a number of effects brought about by reduced PL A₂ activity, including, for example, reduced membrane transport of phospholipids across the gastrointestinal mucosa and/or reduced production of 1-acyl lysophospholipids, such as 1-acyl lysophosphatydylcholine and/or reduced transport of lysophospholipids, 1-acyl lysophosphatydylcholine, that may act as a signaling molecule in subsequent pathways involved in diabetes or other insulin-related conditions.

In some embodiments, a lumen-localized phospholipase inhibitor is used that inhibits phospholipase A2 but does not inhibit or does not significantly inhibit or essentially does not inhibit phospholipase B. In some embodiments, the phospholipase inhibitor inhibits phospholipase A2 but no other gastrointestinal phospholipase, including not inhibiting or not significantly inhibiting or essentially not inhibiting phospholipase A1, and not inhibiting or not significantly inhibiting or essentially not inhibiting phospholipase.

Treatment of Weight-Related Conditions

The term “weight-related conditions” as used herein refers to unwanted weight gain, including overweight, obese and/or hyperlipidemic conditions, and in particular weight gain caused by a high fat or Western diet. Typically, body mass index (BMI) is used as the criteria in determining whether an individual is overweight and/or obese. An adult is considered overweight if, for example, he or she has a body mass index of at least about 25, and is considered obese with a BMI of at least about 30. For children, charts of Body-Mass-Index for Age are used, where a BMI greater than about the 85th percentile is considered “at risk of overweight” and a BMI greater than about the 95th percentile is considered “obese.”

In certain embodiments, a lumen localized phospholipase A2 inhibitor of the present invention can be used to treat weight-related conditions, including unwanted weight gain and/or obesity. In certain embodiments, a lumen localized phospholipase A2 inhibitor decreases fat absorption after a meal typical of a Western diet. In certain embodiments, a lumen localized phospholipase A2 inhibitor increases lipid excretion from a subject on a Western diet. In certain preferred embodiments, the phospholipase inhibitor reduces weight gain in a subject on a (typical) Western diet. In certain embodiments, practice of the present invention can preferentially reduce weight gain in certain tissues and organs, e.g., in some embodiments, a phospholipase A2 inhibitor can decrease weight gain in white fat of a subject on a Western diet.

Without being limited to any particular hypothesis, these benefits may result from a number of effects brought about by reduced PL A₂ activity. For example, inhibition of PL A₂ activity may reduce transport of phospholipids through the gastrointestinal lumen, for example, through the small intestine apical membrane, causing a depletion of the pool of phospholipids (e.g. phosphatidylcholine) in enterocytes, particularly in mammals fed with a high fat diet. In such cases, the de novo synthesis of phospholipids may not be sufficient to sustain the high turnover of phospholipids, e.g. phosphatidylcholine, needed to carry triglycerides, for example by transport in chylomicrons (See Tso, in Fat Absorption, 1986, chapt. 6 177-195, Kuksis A., Ed.), incorporated herein by reference.

PL A₂ inhibition can also reduce production of 1-acyl lysophospholipids, such as 1-acyl lysophosphatydylcholine, that may act as a signaling molecule in subsequent up-regulation pathways of fat absorption, including, for example the release of additional digestive enzymes or hormones, e.g., secretin. See, Huggins, Protection against diet-induced obesity and obesity-related insulin resistance in Group 1B-PL A₂-deficient mice, Am. J. Physiol. Endocrinol. Metab. 283:E994-E1001 (2002), incorporated herein by reference.

Another aspect of the present invention provides composition, kits and methods for reducing or delaying the onset of diet-induced diabetes through weight gain. An unchecked high fat diet can produce not only weight gain, but also can contribute to diabetic insulin resistance. This resistance may be recognized by decreased insulin and leptin levels in a subject. The phospholipase inhibitors, compositions, kits and methods disclosed herein can be used in the prophylactic treatment of diet-induced diabetes, or other insulin-related conditions, e.g. in decreasing insulin and/or leptin levels in a subject on a Western diet.

In some embodiments, a lumen-localized phospholipase inhibitor is used that inhibits phospholipase A2 but does not inhibitor or does not significantly inhibit or essentially does not inhibit phospholipase B. In some embodiments, the phospholipase inhibitor inhibits phospholipase A2 but no other gastrointestinal phospholipase, including not inhibiting or not significantly inhibiting or essentially not inhibiting phospholipase A1, and not inhibiting or not significantly inhibiting or essentially not inhibiting phospholipase B.

Treatment of Cholesterol-Related Conditions

The term “cholesterol-related conditions” as used herein refers to a condition in which modulating the activity of HMG-CoA reductase is desirable and/or modulating the production and/or effects of one or more products of HMG-CoA reductase is desirable. In preferred embodiments, a phospholipase inhibitor of the present invention reduces the activity of HMG-CoA reductase and/or reduces the production and/or effects of one or more products of HMG-CoA reductase. For example, a cholesterol-related condition may involve elevated levels of cholesterol, in particular, non-HDL cholesterol in plasma (e.g., elevated levels of LDL cholesterol and/or VLDL/LDL levels). Typically, a patient is considered to have high or elevated cholesterol levels based on a number of criteria, for example, see Pearlman B L, The New Cholesterol Guidelines, Postgrad Med, 2002; 112(2):13-26, incorporated herein by reference. Guidelines include serum lipid profiles, such as LDL compared with HDL levels.

Examples of cholesterol-related conditions include hypercholesterolemia, lipid disorders such as hyperlipidemia, and atherogenesis and its sequelae of cardiovascular diseases, including atherosclerosis, other vascular inflammatory conditions, myocardial infarction, ischemic stroke, occlusive stroke, and peripheral vascular diseases, as well as other conditions in which decreasing cholesterol can produce a benefit. Other cholesterol-related conditions treatable with compositions, kits, and methods of the present invention include those currently treated with statins, as well as other conditions in which decreasing cholesterol absorption can produce a benefit.

In certain embodiments, a lumen-localized phospholipase inhibitor of the present invention can be used to reduce cholesterol levels, in particular non-HDL plasma cholesterol levels, e.g. by reducing cholesterol absorption. In some preferred embodiments, the composition inhibits phospholipase A2 and at least one other gastrointestinal phospholipase in addition to phospholipase A2, such as preferably phospholipase B, and also such as phospholipase A1, phospholipase C, and/or phospholipase D.

In other embodiments of the invention, the differential activities of phospholipases can be used to treat certain phospholipase-related conditions without undesired side effects resulting from inhibiting other phospholipases. For example, in certain embodiments, a phospholipase inhibitor that inhibits PL A₂, but not inhibiting or not significantly inhibiting or essentially not inhibiting, for example, PLA1, PLB, PLC, or PLD can be used to treat an insulin-related condition (e.g. diabetes) and/or a weight-related condition (e.g. obesity) without affecting, or without significantly affecting, or without essentially effecting, cholesterol absorption of a subject receiving phospholipase inhibiting treatment, e.g., when the subject is on a high fat diet.

Other cholesterol-related conditions of particular interest include dislipidemia conditions, such as hypertriglyceridemia. Hepatic triglyceride synthesis is regulated by available fatty acids, glycogen stores, and the insulin versus glucagon ratio. Patients with a high glucose diet (including, for example, patients on a high-carbohydrate or a high-saccharide diet, and/or patients in a population known to typically consume such diets) are likely to have a balance of hormones that maintains an excess of insulin and also build up glycogen stores, both of which enhance hepatic triglyceride synthesis. In addition, diabetic patients are particularly susceptible, since they are often overweight and are in a state of caloric excess. Hence, the present invention is particularly of interest, in each embodiment herein described, with respect to treatments directed to hypertriglyceridemia.

Without being bound by theory not specifically recited in the claims, the phospholipase A2 inhibitors of the present invention can modulate triglycerides and cholesterol through more than one mechanistic path. For example, the phospholipase A2 inhibitors of the invention can modulate cholesterol absorption and triglyceride absorption from the gastrointestinal tract, and can also modulate the metabolism of fat and glucose, for example, via signaling molecules such as lysophosphatidylcholine (the reaction product of PLA2 catalyzed hydrolysis of phosphatidylcholine), operating directly and/or in conjunction with other hormones such as insulin. Such metabolic modulation can directly impact serum cholesterol and triglyceride levels in patients on a high fat/high disaccharide diet or on a high fat/high carbohydrate diet. VLDL is a lipoprotein packaged by the liver for endogenous circulation from the liver to the peripheral tissues. VLDL contains triglycerides, cholesterol, and phospholipase at its core along with apolipoproteins B100, C1, CII, CIII, and E at its perimeter. Triglycerides make up more than half of VLDL by weight and the size of VLDL is determined by the amount of triglyceride. Very large VLDL is secreted by the liver in states of caloric excess, in diabetes mellitus, and after alcohol consumption, because excess triglycerides are present. As such, inhibition of phospholipase A2 activity can impact metabolism, including for example hepatic triglyceride synthesis. Modulated (e.g., reduced or at least relatively reduced increase) in triglyceride synthesis can provide a basis for modulating serum triglyceride levels and/or serum cholesterol levels, and further can provide a basis for treating hypertriglyceridemia and/or hypercholesterolemia. Such treatments would be beneficial to both diabetic patients (who typically replace their carbohydrate restrictions with higher fat meals), and to hypertriglyceridemic patients (who typically substitute fat with high carbohydrate meals). In this regard, increased protein meals alone are usually not sustainable in the long term for most diabetic and/or hypertriglyceridemic patients.

Moreover, the modulation of serum triglyceride levels can have a beneficial effect on cardiovascular diseases such as atherosclerosis. Triglycerides included in VLDL packaged and released from the liver into circulation are in turn, hydrolyzed by lipoprotein lipase, such that VLDL are converted to VLDL remnants (=IDL). VLDL remnants can either enter the liver (the large ones preferentially do this) or can give rise to LDL. Hence, elevated VLDL in the circulation lowers HDL, which is responsible for reverse cholesterol transport. Since hypertriglyceridemia contributes to elevated LDL levels and also contributes to lowered HDL levels, hypertriglyceridemia is a risk factor for cardiovascular diseases such as atherosclerosis and coronary artery disease (among others, as noted above). Accordingly, modulating hypertriglyceridemia using the phospholipase-A2 inhibitors of the present invention also provide a basis for treating such cardiovascular diseases.

The phospholipase inhibitors, methods, and kits disclosed herein can be used in the treatment of phospholipase-related conditions. In some preferred embodiments, these effects can be realized without a change in diet and/or activity on the part of the subject. For example, the activity of PL A₂ in the gastrointestinal lumen may be inhibited to result in a decrease in fat absorption and/or a reduction in weight gain in a subject on a Western diet compared to if the subject was not receiving PL A₂ inhibiting treatment. More preferably, this decrease and/or reduction occurs without a change, without a significant change, or essentially without a change, in energy expenditure and/or food intake on the part of the subject, and without a change, or without a significant change, or essentially without a change in the body temperature of the subject. Further, in preferred embodiments, a phospholipase inhibitor of the present invention can be used to offset certain negative consequences of high fat diets without affecting normal aspects of metabolism on non-high fat diets.

The present invention also includes kits that can be used to treat phospholipase-related conditions, preferably phospholipase A2-related conditions or phospholipase-related conditions induced by diet, including, but not limited to, insulin-related conditions (e.g., diabetes, particularly diabetes type 2), weight-related conditions (e.g., obesity) and/or cholesterol-related conditions. These kits comprise at least one composition of the present invention and instructions teaching the use of the kit according to the various methods described herein.

Treatments Using Inhibitors Comprising Fused Five-and-Six-Membered Rings

In some preferred embodiments, phospholipase-related conditions can be treated (especially diet-related conditions prevalent in populations consuming high-fat diets, and therefore being at risk of diet-induced conditions such as obesity, diabetes, insulin resistance, and glucose intolerance) using lumen-localized inhibitors comprising a small organic molecule phospholipase inhibitor or inhibiting moiety that comprises or is derived from a substituted organic compound having a fused five-member ring and six-member ring, and preferably a fused five-member ring and six-member ring having one or more heteroatoms (e.g., nitrogen, oxygen) substituted within the ring structure of the five-member ring, within the ring structure of the six-member ring, or within the ring structure of each of the five-member and six-member rings. In each case the inhibiting moiety can comprise substituent groups effective for imparting phospholipase inhibiting functionality to the moiety. The inhibiting moiety can also include a substituent having functionality for linking directly or indirectly to the polymer moiety. In especially preferred embodiments, phospholipase-related conditions can be treated using a phospholipase inhibitor or inhibiting moiety that comprises an indole moiety, such as a substituted indole moiety. Such small molecule inhibitors or inhibiting moieties have been found to be especially effective in treating phospholipase-related conditions. (See, for example, PCT Appl. No. US/2005/015416 entitled “Treatment of Diet-Related Conditions Using Phospholipase-A2 Inhibitors Comprising Indoles and Related Compounds” filed on May 3, 2005 by Buysse et al.; See also PCT Appl. No. US/2005/015281 entitled “Treatment Hypercholesterolemia, Hypertriglyceridemia and Cardovascular-Related Conditions Using Phospholipase-A2 Inhibitors” filed on May 3, 2005 by Charmot et al., each of which is incorporated herein by reference).

Inhibitor Formulations, Routes of Administration, and Effective Doses

The phospholipase inhibitors useful in the present invention, or pharmaceutically acceptable salts thereof, can be delivered to a patient using a number of routes or modes of administration. The term “pharmaceutically acceptable salt” means those salts which retain the biological effectiveness and properties of the compounds used in the present invention, and which are not biologically or otherwise undesirable. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compounds used in the present invention contain a carboxyl group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

If necessary or desirable, the phospholipase inhibitor may be administered in combination with one or more other therapeutic agents. The choice of therapeutic agent that can be co-administered with a composition of the invention will depend, in part, on the condition being treated. For example, for treating obesity, or other weight-related conditions, a phospholipase inhibitor of some embodiments of the present invention can be used in combination with a statin, a fibrate, a bile acid binder, an ezitimibe (e.g., Zetia, etc), a saponin, a lipase inhibitor (e.g. Orlistat, etc), and/or an appetite suppressant, and the like. With respect to treating insulin-related conditions, e.g., diabetes, a phospholipase inhibitor of some embodiments the present invention can be used in combination with a biguanide (e.g., Metformin), thiazolidinedione, and/or α-glucosidase inhibitor, and the like.

The phospholipase inhibitors (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions for use in accordance with the present invention may be formulated in conventional manner using one or more physiologically acceptable carriers compromising excipients and auxiliaries which facilitate processing of the active compounds into preparations which can be used pharmaceutically. Proper formulation is dependent upon the route of administration chosen.

The phospholipase inhibitors can be administered by direct placement, orally, and/or rectally. Preferably, the phospholipase inhibitor or the pharmaceutical composition comprising the phospholipase inhibitor is administered orally. The oral form in which the phospholipase inhibitor is administered can include a powder, tablet, capsule, solution, or emulsion. The effective amount can be administered in a single dose or in a series of doses separated by appropriate time intervals, such as hours.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions, wafers, and the like, for oral ingestion by a patient to be treated. In some embodiments, the inhibitor may be formulated as a sustained release preparation. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. In some embodiments, the oral formulation does not have an enteric coating.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

Suitable carriers used in formulating liquid dosage forms for oral as well as parenteral administration include non-aqueous, pharmaceutically-acceptable polar solvents such as hydrocarbons, alcohols, amides, oils, esters, ethers, ketones, and/or mixtures thereof, as well as water, saline solutions, electrolyte solutions, dextrose solutions (e.g., DW5), and/or any other aqueous, pharmaceutically acceptable liquid.

Suitable nonaqueous, pharmaceutically-acceptable polar solvents include, but are not limited to, alcohols (e.g., aliphatic or aromatic alcohols having 2-30 carbon atoms such as methanol, ethanol, propanol, isopropanol, butanol, t-butanol, hexanol, octanol, benzyl alcohol, amylene hydrate, glycerin (glycerol), glycol, hexylene glycol, lauryl alcohol, cetyl alcohol, stearyl alcohol, tetrahydrofurfuryl alcohol, fatty acid esters of fatty alcohols such as polyalkylene glycols (e.g., polyethylene glycol and/or polypropylene glycol), sorbitan, cholesterol, sucrose and the like); amides (e.g., dimethylacetamide (DMA), benzyl benzoate DMA, N,N-dimethylacetamide amides, 2-pyrrolidinone, polyvinylpyrrolidone, 1-methyl-2-pyrrolidinone, and the like); esters (e.g., 2-pyrrolidinone, 1-methyl-2-pyrrolidinone, acetate esters (such as monoacetin, diacetin, and triacetin and the like), and the like, aliphatic or aromatic esters (such as dimethylsulfoxide (DMSO), alkyl oleate, ethyl caprylate, ethyl benzoate, ethyl acetate, octanoate, benzyl benzoate, benzyl acetate, esters of glycerin such as mono, di, or tri-glyceryl citrates or tartrates, ethyl carbonate, ethyl oleate, ethyl lactate, N-methylpyrrolidinone, fatty acid esters such as isopropyl myristrate, fatty acid esters of sorbitan, glyceryl monostearate, glyceride esters such as mono, di, or tri-glycerides, fatty acid derived PEG esters such as PEG-hydroxystearate, PEG-hydroxyoleate, and the like, pluronic 60, polyoxyethylene sorbitol oleic polyesters, polyoxyethylene sorbitan esters such as polyoxyethylene-sorbitan monooleate, polyoxyethylene-sorbitan monostearate, polyoxyethylene-sorbitan monolaurate, polyoxyethylene-sorbitan monopalmitate, alkyleneoxy modified fatty acid esters such as polyoxyl 40 hydrogenated castor oil and polyoxyethylated castor oils, saccharide fatty acid esters (i.e., the condensation product of a monosaccharide, disaccharide, or oligosaccharide or mixture thereof with a fatty acid(s) (e.g., saturated fatty acids such as caprylic acid, myristic acid, palmitic acid, capric acid, lauric acid, and stearic acid, and unsaturated fatty acids such as palmitoleic acid, oleic acid, elaidic acid, erucic acid and linoleic acid)), or steroidal esters and the like); alkyl, aryl, or cyclic ethers (e.g., diethyl ether, tetrahydrofuran, diethylene glycol monoethyl ether, dimethyl isosorbide and the like); glycofurol (tetrahydrofurfuryl alcohol polyethylene glycol ether); ketones (e.g., acetone, methyl isobutyl ketone, methyl ethyl ketone and the like); aliphatic, cycloaliphatic or aromatic hydrocarbons (e.g., benzene, cyclohexane, dichloromethane, dioxolanes, hexane, n-hexane, n-decane, n-dodecane, sulfolane, tetramethylenesulfoxide, tetramethylenesulfon, toluene, tetramethylenesulfoxide dimethylsulfoxide (DMSO) and the like); oils of mineral, animal, vegetable, essential or synthetic origin (e.g., mineral oils such as refined paraffin oil, aliphatic or wax-based hydrocarbons, aromatic hydrocarbons, mixed aliphatic and aromatic based hydrocarbons, and the like, vegetable oils such as linseed, soybean, castor, rapeseed, coconut, tung, safflower, cottonseed, groundnut, palm, olive, corn, corn germ, sesame, persic, peanut oil, and the like, as well as glycerides such as mono-, di- or triglycerides, animal oils such as cod-liver, haliver, fish, marine, sperm, squalene, squalane, polyoxyethylated castor oil, shark liver oil, oleic oils, and the like); alkyl or aryl halides e.g., methylene chloride; monoethanolamine; trolamine; petroleum benzin; omega-3 polyunsaturated fatty acids (e.g., α-linolenic acid, docosapentaenoic acid, docosahexaenoic acid, eicosapentaenoic acid, and the like); polyglycol ester of 12-hydroxystearic acid; polyethylene glycol; polyoxyethylene glycerol, and the like.

Other pharmaceutically acceptable solvents that can be used in formulating pharmaceutical compositions of a phospholipase inhibitor of the present invention including, for example, for direct placement, are well known to those of ordinary skill in the art, e.g. see Modern Pharmaceutics, (G. Banker et al., eds., 3d ed.) (Marcel Dekker, Inc., New York, N.Y., 1995), The Handbook of Pharmaceutical Excipients, (American Pharmaceutical Association, Washington, D.C.; The Pharmacological Basis of Therapeutics, (Goodman & Gilman, McGraw Hill Publishing), Remington's Pharmaceutical Sciences (A. Gennaro, ed., 19th ed.) (Mack Publishing, Easton, Pa., 1995), Pharmaceutical Dosage Forms, (H. Lieberman et al., eds.,) (Marcel Dekker, Inc., New York, N.Y., 1980); and The United States Pharmacopeia 24, The National Formulary 19, (National Publishing, Philadelphia, Pa., 2000).

Formulations for rectal administration may be prepared in the form of a suppository, an ointment, an enema, a tablet, or a cream for release of the phospholipase inhibitor in the gastrointestinal tract, e.g., the small intestine. Rectal suppositories can be made by mixing one or more phospholipase inhibitors of the present invention, or pharmaceutically acceptable salts thereof, with acceptable vehicles, for example, cocoa butter, with or without the addition of waxes to alter melting point. Acceptable vehicles can also include glycerin, salicylate and/or polyethylene glycol, which is solid at normal storage temperature, and a liquid at those temperatures suitable to release the phospholipase inhibitor inside the body, such as in the rectum. Oils may also be used in rectal formulations of the soft gelatin type and in suppositories. Water soluble suppository bases, such as polyethylene glycols of various molecular weights, may also be used. Suspension formulations may be prepared that use water, saline, aqueous dextrose and related sugar solutions, and glycerols, as well as suspending agents such as pectins, carbomers, methyl cellulose, hydroxypropyl cellulose or carboxymethyl cellulose, as well as buffers and preservatives.

Pharmaceutical compositions suitable for use in the present invention include compositions wherein the active ingredients are present in an effective amount, i.e., in an amount sufficient to produce a therapeutic and/or a prophylactic benefit in at least one condition being treated. The actual amount effective for a particular application will depend on the condition being treated and the route of administration. Determination of an effective amount is well within the capabilities of those skilled in the art, especially in light of the disclosure herein. For example, the IC50 values and ranges provided in Table 1 above provide guidance to enable one of ordinary skill in the art to select effective dosages of the corresponding phospholipase inhibiting moieties.

The effective amount when referring to a phospholipase inhibitor will generally mean the dose ranges, modes of administration, formulations, etc., that have been recommended or approved by any of the various regulatory or advisory organizations in the medical or pharmaceutical arts (eg, FDA, AMA) or by the manufacturer or supplier. Effective amounts of phospholipase inhibitors can be found, for example, in the Physicians Desk Reference. The effective amount when referring to producing a benefit in treating a phospholipase-related condition, such as insulin-related conditions (e.g., diabetes), weight-related conditions (e.g., obesity), and/or cholesterol related-conditions will generally mean the levels that achieve clinical results recommended or approved by any of the various regulatory or advisory organizations in the medical or pharmaceutical arts (eg, FDA, AMA) or by the manufacturer or supplier.

A person of ordinary skill using techniques known in the art can determine the effective amount of the phospholipase inhibitor. In the present invention, the effective amount of a phospholipase inhibitor localized in the gastrointestinal lumen can be less than the amount administered in the absence of such localization. Even a small decrease in the amount of phospholipase inhibitor administered is considered useful for the present invention. A significant decrease or a statistically significant decrease in the effective amount of the phospholipase inhibitor is particularly preferred. In some embodiments of the invention, the phospholipase inhibitor reduces activity of phospholipase to a greater extent compared to non-lumen localized inhibitors. Lumen-localization of the phospholipase inhibitor can decrease the effective amount necessary for the treatment of phospholipase-related conditions, such as insulin-related conditions (e.g., diabetes), weight-related conditions (e.g., obesity) and/or cholesterol-related conditions by about 5% to about 95%. The amount of phospholipase inhibitor used could be the same as the recommended dosage or higher than this dose or lower than the recommended dose.

In some embodiments, the recommended dosage of a phospholipase inhibitor is between about 0.1 mg/kg/day and about 1,000 mg/kg/day. The effective amount for use in humans can be determined from animal models. For example, a dose for humans can be formulated to achieve circulating and/or gastrointestinal concentrations that have been found to be effective in animals, e.g. a mouse model as the ones described in the samples below.

A person of ordinary skill in the art can determine phospholipase inhibition by measuring the amount of a product of a phospholipase, e.g., lysophosphatidylcholine (LPC), a product of PL A₂. The amount of LPC can be determined, for example, by measuring small intestine, lymphatic, and/or serum levels post-prandially. Another technique for determining amount of phospholipase inhibition involves taking direct fluid samples from the gastrointestinal tract. A person of ordinary skill in the art would also be able to monitor in a patient the effect of a phospholipase inhibitor of the present invention, e.g., by monitoring cholesterol and/or triglyceride serum levels. Other techniques would be apparent to one of ordinary skill in the art. Other approaches for measuring phospholipase inhibition and/or for demonstrating the effects of phospholipase inhibitors of some embodiments are further illustrated in the examples below.

### EXAMPLES

### Example 1A

### SYNTHESIS OF ILY-4001 [2-(3-(2-AMINO-2-OXOACETYL)-1-(BIPHENYL-2-YLMETHYL)-2-METHYL-1H-INDOL-4-YLOXY)ACETIC ACID]

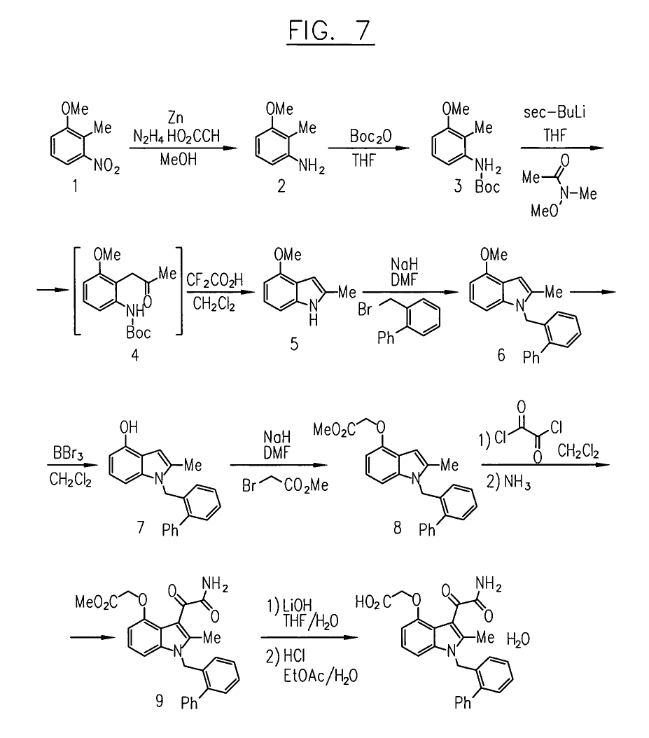

This example synthesized a compound for use as a phospholipase inhibitor or inhibiting moiety. Specifically, the compound 2-(3-(2-amino-2-oxoacetyl)-1-(biphenyl-2-ylmethyl)-2-methyl-1H-indol-4-yloxy)acetic acid, shown in FIG. 7 was synthesized. This compound is designated in these examples as ILY-4001, and is alternatively referred to herein as methyl indoxam.

Reference is made to FIG. 7, which outlines the overall synthesis scheme for ILY-4001. The numbers under each compound shown in FIG. 7 correspond to the numbers in parenthesis associated with the chemical name for each compound in the following experimental description.

2-Methyl-3-methoxyaniline (2) [04-035-11]. To a stirred cooled (ca. 5° C.) hydrazine hydrate (159.7 g, 3.19 mol), 85% formic acid (172.8 g, 3.19 mol) was added drop wise at 10-20° C. The resultant mixture was added drop wise to a stirred suspension of zinc dust (104.3 g, 1.595 mol) in a solution of 2-methyl-3-nitroanisole (1) (53.34 g, 0.319 mol) in methanol (1000 mL). An exothermic reaction occurred. After the addition was complete, the reaction mixture was stirred for additional 2 h (until temperature dropped from 61° C. to RT) and the precipitate was filtered off and washed with methanol (3×150 mL). The filtrate was concentrated under reduced pressure to a volume of ca. 250 mL. The residue was treated with EtOAc (500 ml) and saturated aqueous NaHCO₃ (500 mL). The aqueous phase was separated off and discarded. The organic phase was washed with water (300 mL) and extracted with 1N HCl (800 mL). The acidic extract was washed with EtOAc (300 mL) and was basisified with K₂CO₃ (90 g). The free base 2 was extracted with EtOAc (3×200 mL) and the combined extracts were dried over MgSO₄. After filtration and removal of the solvent from the filtrate, product 2 was obtained as a red oil, which was used in the next step without further purification. Yield: 42.0 g (96%).

N-tert-Butyloxycarbonyl-2-methyl-3-methoxyaniline (3) [04-035-12]. A stirred solution of amine 2 (42.58 g, 0.31 mol) and di-tert-butyl dicarbonate (65.48 g, 0.30 mol) in THF (300 mL) was heated to maintain reflux for 4 h. After cooling to RT, the reaction mixture was concentrated under reduced pressure and the residue was dissolved in EtOAc (500 mL). The resultant solution was washed with 0.5 M citric acid (2×100 mL), water (100 mL), saturated aqueous NaHCO₃ (200 mL), brine (200 mL) and dried over MgSO₄. After filtration and removal of the solvent from the filtrate, the residue (red oil, 73.6 g) was dissolved in hexanes (500 mL) and filtered through a pad of Silica Gel (for TLC). The filtrate was evaporated under reduced pressure to provide N-Boc aniline 3 as a yellow solid. Yield: 68.1 g (96%).

4-Methoxy-2-methyl-1H-indole (5) [04-035-13]. To a stirred cooled (−50° C.) solution of N-Boc aniline 3 (58.14 g, 0.245 mol) in anhydrous THF (400 mL), a 1.4 M solution of sec-BuLi in cyclohexane (0.491 mol, 350.7 mL) was added drop wise at −48-−50° C. and the reaction mixture was allowed to warm up to −20° C. After cooling to −60° C., a solution of N-methoxy-N-methylacetamide (25.30 g, 0.245 mol) in THF (25 mL) was added drop wise at −57-−60° C. The reaction mixture was stirred for 1 h at −60° C. and was allowed to warm up to 15° C. during 1 h. After cooling to −15° C., the reaction was quenched with 2N HCl (245 mL) and the resultant mixture was adjusted to pH of ca. 7 with 2N HCl. The organic phase was separated off and saved. The aqueous phase was extracted with EtOAc (3×100 mL). The organic solution was concentrated under reduced pressure and the residual pale oil was dissolved in EtOAc (300 mL) and combined with the EtOAc extracts. The resultant solution was washed with water (2×200 mL), 0.5 M citric acid, (100 mL), saturated aqueous NaHCO₃ (100 mL), brine (200 mL) and dried over MgSO₄. After filtration and removal of the solvent from the filtrate, a mixture of starting N-Boc aniline 3 and intermediate ketone 4 (ca. 1:1 mol/mol) was obtained as a pale oil (67.05 g).

The obtained oil was dissolved in anhydrous CH₂Cl₂ (150 mL) and the solution was cooled to 0-−5° C. Trifluoroacetic acid (65 mL) was added drop wise and the reaction mixture was allowed to warm up to RT. After 16 h of stirring, an additional portion of trifluoroacetic acid (35 mL) was added and stirring was continued for 16 h. The reaction mixture was concentrated under reduced pressure and the red oily residue was dissolved in CH₂Cl₂ (500 mL). The resultant solution was washed with water (3×200 mL) and dried over MgSO₄. Filtration through a pad of Silica Gel 60 and evaporation of the filtrate under reduced pressure provided crude product 5 as a yellow solid (27.2 g). Purification by dry chromatography (Silica Gel for TLC, 20% EtOAc in hexanes) afforded indole 5 as a white solid. Yield: 21.1 g (53%)

1-[(1,1′-Biphenyl)-2-ylmethyl]-4-methoxy-2-methyl-1H-indole (6) [04-035-14]. A solution of indole 5 (16.12 g, 0.10 mol) in anhydrous DMF (100 mL) was added drop wise to a stirred cooled (ca. 15° C.) suspension of sodium hydride (0.15 mol, 6.0 g, 60% in mineral oil, washed with 100 mL of hexanes before the reaction) in DMF (50 mL) and the reaction mixture was stirred for 0.5 h at RT. After cooling the reaction mixture to ca. 5° C., 2-phenylbenzyl bromide (25.0 g, 0.101 mol) was added drop wise and the reaction mixture was stirred for 18 h at RT. The reaction was quenched with water (10 mL) and EtOAc (500 mL) was added. The resultant mixture was washed with water (2×200 mL+3×100 mL), brine (200 mL) and dried over MgSO₄. After filtration and removal of the solvent from the filtrate under reduced pressure, the residue (35.5 g, thick red oil) was purified by dry chromatography (Silica Gel for TLC, 5%→25% CH₂Cl₂ in hexanes) to afford product 6 as a pale oil. Yield: 23.71 g (72%).

1-[(1,1′-Biphenyl)-2-ylmethyl]-4-hydroxy-2-methyl-1H-indole (7) [04-035-15]. To a stirred cooled (ca. 10° C.) solution of the methoxy derivative 6 (23.61 g, 72.1 mmol) in anhydrous CH₂Cl₂ (250 mL), a 1M solution of BBr₃ in CH₂Cl₂ (300 mmol, 300 mL) was added drop wise at 15-20° C. and the dark reaction mixture was stirred for 5 h at RT. After concentrating of the reaction mixture under reduced pressure, the dark oily residue was cooled to ca. 5° C. and was dissolved in precooled (15° C.) EtOAc (450 mL). The resultant cool solution was washed with water (3×200 mL), brine (200 mL) and dried over MgSO₄. After filtration and removal of the solvent from the filtrate under reduced pressure, the residue (26.1 g, dark semi-solid) was purified by dry chromatography (Silica Gel for TLC, 5%→25% EtOAc in hexanes) to afford product 7 as a brown solid. Yield: 4.30 g (19%)

2-{1-[(1,1′-Biphenyl)-2-ylmethyl)-2-methyl-1H-indol-4-yl]oxy}-acetic acid methyl ester (8) [04-035-16]. To a stirred suspension of sodium hydride (0.549 g, 13.7 mmol, 60% in mineral oil) in anhydrous DMF (15 mL), a solution of compound 7 (4.30 g, 13.7 mmol) in DMF (30 mL) was added drop wise and the resultant mixture was stirred for 40 min at RT. Methyl bromoacetate (2.10 g, 13.7 mmol) was added drop wise and stirring was continued for 21 h at RT. The reaction mixture was diluted with EtOAc (200 mL) and washed with water (4×200 mL), brine (200 mL) and dried over MgSO₄. After filtration and removal of the solvent from the filtrate under reduced pressure, the residue (5.37 g, dark semi-solid) was purified by dry chromatography (Silica Gel for TLC, 5%→30% EtOAc in hexanes) to afford product 8 as a yellow solid. Yield: 4.71 g (89%).

2-{[3-(2-Amino-1,2-dioxoethyl)-1-[(1,1′-biphenyl)-2-ylmethyl)-2-methyl-1H-indol-4-yl]oxy}-acetic acid methyl ester (9) [04-035-17]. To a stirred solution of oxalyl chloride (1.55 g, 12.2 mmol) in anhydrous CH₂Cl₂ (20 mL), a solution of compound 8 in CH₂Cl₂ (40 mL) was added drop wise and the reaction mixture was stirred for 80 min at RT. After cooling the reaction mixture to −10° C., a saturated solution of NH₃ in CH₂Cl₂ (10 mL) was added drop wise and then the reaction mixture was saturated with NH₃ (gas) at ca. 0° C. Formation of a precipitate was observed. The reaction mixture was allowed to warm up to RT and was concentrated under reduced pressure to dryness. The dark solid residue (6.50 g) was subjected to dry chromatography (Silica Gel for TLC, 30% EtOAc in hexanes→100% EtOAc) to afford product 9 as a yellow solid. Yield: 4.64 g (83%).

2-{[3-(2-Amino-1,2-dioxoethyl)-1-[(1,1′-biphenyl)-2-ylmethyl)-2-methyl-1H-indol-4-yl]oxy}-acetic acid (ILY-4001) [04-035-18]. To a stirred solution of compound 9 (4.61 g, 10.1 mmol) in a mixture of THF (50 mL) and water (10 mL), a solution of lithium hydroxide monohydrate (0.848 g, 20.2 mmol) in water (20 mL) was added portion wise and the reaction mixture was stirred for 2 h at RT. After addition of water (70 mL), the reaction mixture was concentrated under reduced pressure to a volume of ca. 100 mL. Formation of a yellow precipitate was observed. To the residual yellow slurry, 2N HCl (20 mL) and EtOAc (200 mL) were added and the resultant mixture was stirred for 16 h at RT. The yellowish-greenish precipitate was filtered off and washed with EtOAc (3×20 mL), Et₂O (20 mL) and hexanes (20 mL). After drying in vacuum, the product (2.75 g) was obtained as a pale solid. MS: 443.27 (M⁺+1). Elemental Analysis: Calcd for C₂₆H₂₂N₂O₅+H₂O: C, 67.82; H, 5.25; N, 6.08. Found: C, 68.50; H, 4.96; N, 6.01. HPLC: 96.5% purity. ¹H NMR (DMSO-d₆) 7.80 (br s, 1H), 7.72-7.25 (m, 9H), 7.07 (t, 1H), 6.93 (d, 1H), 6.57 (d, 1H), 6.43 (d, 1H), 5.39 (s, 2H), 4.68 (s, 2H), 2.38 (s, 3H).

The aqueous phase of the filtrate was separated off and the organic one was washed with brine (100 mL) and dried over MgSO₄. After filtration and removal of the solvent from the filtrate under reduced pressure, the greenish solid residue was washed with EtOAc (3×10 mL), Et₂O (10 mL) and hexanes (10 mL). After drying in vacuum, an additional portion (1.13 g) of product was obtained as a greenish solid.

Total yield: 2.75 g+1.13 g=3.88 g (87%).

### Example 1B

### CHARACTERIZATION STUDIES—ILY-4001 [2-(3-(2-AMINO-2-OXOACETYL)-1-(BIPHENYL-2-YLMETHYL)-2-METHYL-1H-INDOL-4-YLOXY)ACETIC ACID]

This example characterized ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam, with respect to activity, as determined by IC50 assay (Example 1B-1), with respect to cell absorbtion, as determined by in-vitro Caco-2 assay (Example 1B-2) and with respect to bioavailability, as determined using in-vivo mice studies (Example 1B-3).

### Example 1B-1

### IC-50 STUDY—ILY-4001 [2-(3-(2-AMINO-2-OXOACETYL)-1-(BIPHENYL-2-YLMETHYL)-2-METHYL-1H-INDOL-4-YLOXY)ACETIC ACID]

This example evaluated the IC50 activity value of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam.

A continuous fluorimetric assay for PLA2 activity described in the literature was used to determine IC (Leslie, C C and Gelb, M H (2004) Methods in Molecular Biology “Assaying phospholipase A2 activity”, 284: 229-242, Singer, A G, et al. (2002) Journal of Biological Chemistry “Interfacial kinetic and binding properties of the complete set of human and mouse groups I, II, V, X, and XII secreted phospholipases A2”, 277: 48535-48549, Bezzine, S, et al. (2000) Journal of Biological Chemistry “Exogenously added human group X secreted phospholipase A(2) but not the group IB, IIA, and V enzymes efficiently release arachidonic acid from adherent mammalian cells”, 275: 3179-3191) and references therein.

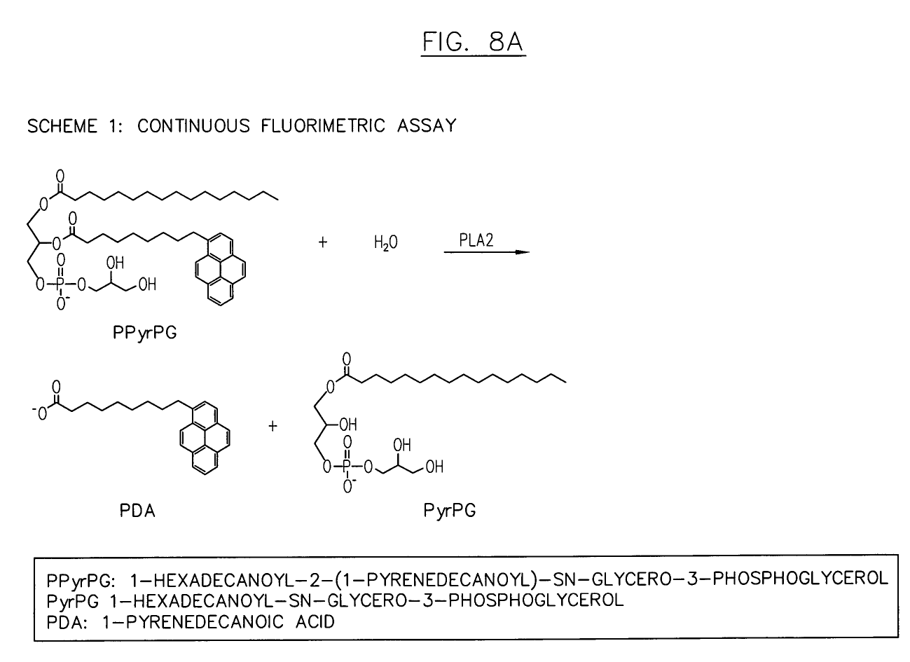

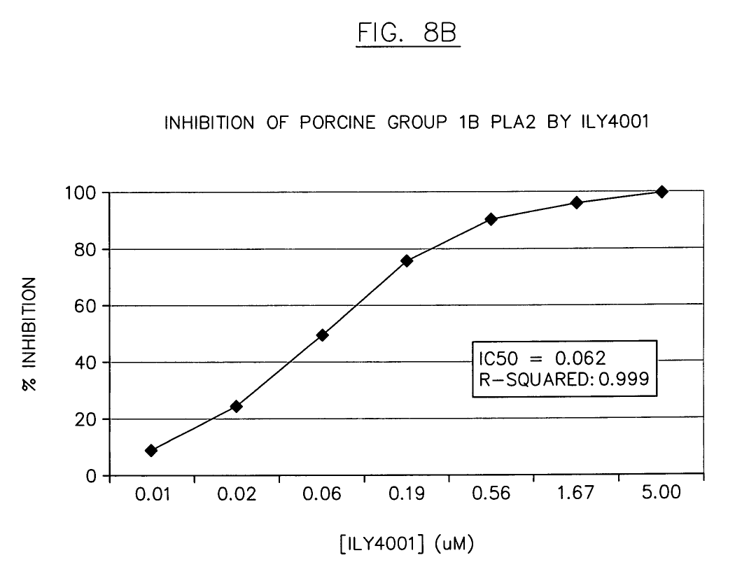

Generally, this assay used a phosphatidylglycerol (or phosphatidylmethanol) substrate with a pyrene fluorophore on the terminal end of the sn-2 fatty acyl chain. Without being bound by theory, close proximity of the pyrenes from neighboring phospholipids in a phospholipid vesicle caused the spectral properties to change relative to that of monomeric pyrene. Bovine serum albumin was present in the aqueous phase and captured the pyrene fatty acid when it is liberated from the glycerol backbone owing to the PLA2-catalyzed reaction. In this assay, however, a potent inhibitor can inhibit the liberation of pyrene fatty acid from the glycerol backbone. Hence, such features allow for a sensitive PLA2 inhibition assay by monitoring the fluorescence of albumin-bound pyrene fatty acid, as represented in Scheme 1 shown in FIG. 8A. The effect of a given inhibitor and inhibitor concentration on any given phospholipase can be determined.

In this example, the following reagents and equipment were obtained from commercial vendors:
1. Porcine PLA2 IB
2. 1-hexadecanoyl-2-(1-pyrenedecanoyl)-sn-glycero-3-phosphoglycerol (PPyrPG)
3. 1-hexadecanoyl-2-(1-pyrenedecanoyl)-sn-glycero-3-phosphomethanol (PPyrPM)
4. Bovine serum albumin (BSA, fatty acid free)
5. 2-Amino-2-(hydroxymethyl)-1,3-propanediol, hydrochloride (Tris-HCl)
6. Calcium chloride
7. Potassium chloride
8. Solvents: DMSO, toluene, isopropanol, ethanol
9. Molecular Devices SPECTRAmax microplate spectrofluorometer
10. Costar 96 well black wall/clear bottom plate

In this example, the following reagents were prepared:
1. PPyrPG (or PPyrPM) stock solution (1 mg/ml) in toluene:isopropanol (1:1)
2. Inhibitor stock solution (10 mM) in DMSO
3. 3% (w/v) bovine serum albumin (BSA)
4. Stock buffer: 50 mM Tris-HCl, pH 8.0, 50 mM KCl and 1 mM CaCl₂

In this example, the procedure was performed as follows:
1. An assay buffer was prepared by adding 3 ml 3% BSA to 47 ml stock buffer.
2. Solution A was prepared by adding serially diluted inhibitors to the assay buffer. Inhibitor were three-fold diluted in a series of 8 from 15 μM.
3. Solution B was prepared by adding PLA2 to the assay buffer. This solution was prepared immediately before use to minimize enzyme activity loss.
4. Solution C was prepared by adding 30 ul PPyrPG stock solution to 90 ul ethanol, and then all 120 ul of PPyrPG solution was transferred drop-wise over approximately 1 min to the continuously stirring 8.82 ml assay buffer to form a final concentration of 4.2 uM PPyrPG vesicle solution.
5. The SPECTRAmax microplate spectrofluorometer was set at 37° C.
6. 100 ul of solution A was added to each inhibition assay well of a costar 96 well black wall/clear bottom plate
7. 100 ul of solution B was added to each inhibition assay well of a costar 96 well black wall/clear bottom plate.
8. 100 ul of solution C was added to each inhibition assay well of a costar 96 well black wall/clear bottom plate.
9. The plate was incubated inside the spectrofluorometer chamber for 3 min.
10. The fluorescence was read using an excitation of 342 nm and an emission of 395 nm.

In this example, the IC50 was calculated using the BioDataFit 1.02 (Four Parameter Model) software package. The equation used to generate the curve fit is:

$${y}_{j} = β + \frac{α - β}{1 + exp ( - κ ( log ( {x}_{j} ) - γ ) )}$$

wherein: α is the value of the upper asymptote; β is the value of the lower asymptote; κ is a scaling factor; γ is a factor that locates the x-ordinate of the point of inflection at

$$exp [ \frac{κγ - log ( \frac{1 + κ}{κ - 1} )}{κ} ]$$

with constraints α, β, κ, γ>0, β<α, and β<γ<α.

The results, shown in FIG. 8B, indicate that the concentration of ILY4001 resulting in 50% maximal PLA2 activity was calculated to be 0.062 uM.

### Example 1B-2

### CACO-2 ABSORBTION STUDY—ILY-4001 [2-(3-(2-AMINO-2-OXOACETYL)-1-(BIPHENYL-2-YLMETHYL)-2-METHYL-1H-INDOL-4-YLOXY)ACETIC ACID]

This example evaluated the intestinal absorption of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam using in-vitro assays with Caco-2 cells.

Briefly, the human colon adenocarcinoma cell line, Caco-2, was used to model intestinal drug absorption. It has been shown that the apparent permeability values measured in Caco-2 monolayers in the range of 1×10⁻⁷ cm/sec or less typically correlate with relatively poor human absorption. (Artursson, P., K. Palm, et al. (2001). “Caco-2 monolayers in experimental and theoretical predictions of drug transport.” Adv Drug Deliv Rev 46(1-3): 27-43.).

In order to determine the compound permeability, Caco-2 cells (ATCC) were seeded into 24-well transwells (Costar) at a density of 6×10⁴ cells/cm². Monolayers were grown and differentiated in MEM (Mediatech) supplemented with 20% FBS, 100 U/ml penicillin, and 100 ug/ml streptomycin at 37° C., 95% humidity, 95% air, and 5% CO₂. The culture medium was refreshed every 48 hours. After 21 days, the cells were washed in transport buffer made up of HBSS with HEPES and the monolayer integrity was evaluated by measuring the trans-epithelial electrical resistance (TEER) of each well. Wells with TEER values of 350 ohm-cm² or better were assayed.

ILY-4001 and Propranolol (a transcellular transport control) were diluted to 50 ug/ml in transport buffer and added to the apical wells separately. 150 ul samples were collected for LC/MS analysis from the basolateral well at 15 min, 30 min, 45 min, 1 hr, 3 hr, and 6 hr time points; replacing the volume with pre-warmed transport buffer after each sampling. The apparent permeabilities in cm/s were calculated based on the equation:
Pₐₚₚ=(dQ/dt)×(1/C₀)×(1/A)
Where dQ/dt is the permeability rate corrected for the sampling volumes over time, C₀ is the initial concentration, and A is the surface area of the monolayer (0.32 cm²). At the end of the experiment, TEER measurements were retaken and wells with readings below 350 ohm-cm² indicated diminished monolayer integrity such that the data from these wells were not valid for analysis. Finally, wells were washed with transport buffer and 100 uM of Lucifer Yellow was added to the apical wells. 15 min, 30 min, and 45 min time points were sampled and analyzed by LC/MS to determine paracellular transport.

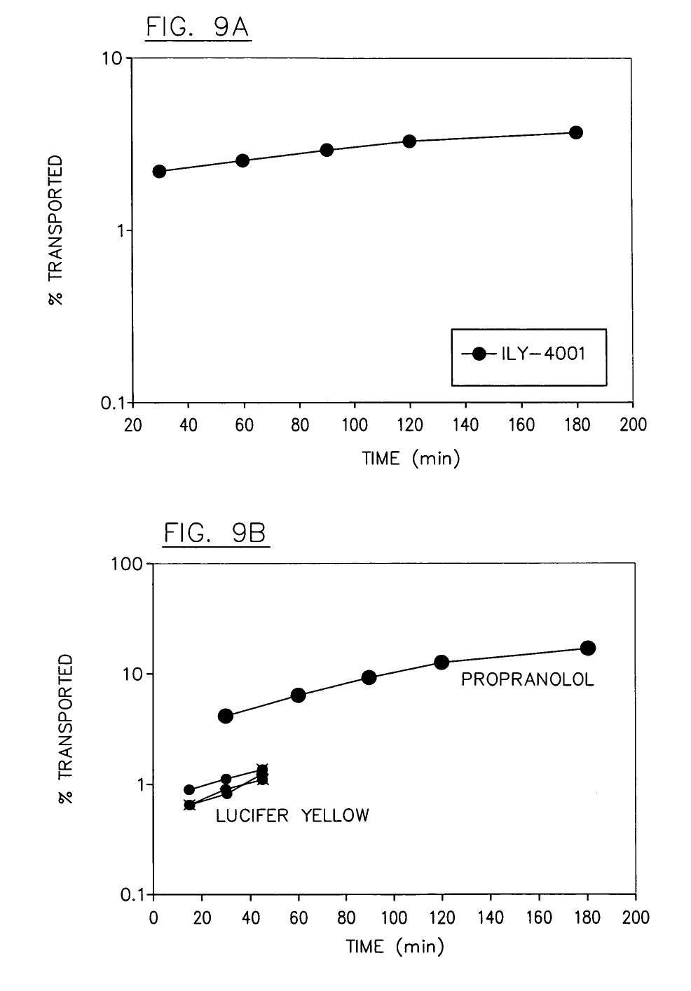

Results from the Caco-2 permeability study for ILY-4001 are shown in FIG. 9A, in which the apparent permeability (cm/s) for ILY-4001 was determined to be around 1.66×10⁻⁷. The results for Lucifer Yellow and Propranolol permeability as paracellular and transcellular transport controls were also determined, and are shown in FIG. 9B, with determined apparent permeability (cm/s) of around 1.32×10⁻⁵ for Propranolol and around 2.82×10⁻⁷+/−0.37×10⁻⁷ for Lucifer Yellow.

### Example 1B-3

### PHARMOKINETIC STUDY—ILY-4001 [2-(3-(2-AMINO-2-OXOACETYL)-1-(BIPHENYL-2-YLMETHYL)-2-METHYL-1H-INDOL-4-YLOXY)ACETIC ACID]

This example evaluated the bioavailability of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam. Specifically, a pharmokinetic study was conducted to determine the fraction of unchanged ILY-4001 in systemic circulation following administration.

Bioavailability was calculated as a ratio of AUC-oral/AUC-intravenous (IV). To determine this ratio, a first set of subject animals were given a measured intravenous (IV) dose of ILY-4001, followed by a determination of ILY-4001 levels in the blood at various time points after administration (e.g., 5 minutes through 24 hours). Another second set of animals was similarly dosed using oral administration, with blood levels of ILY-4001 determined at various time points after administration (e.g., 30 minutes through 24 hours). The level of ILY-4001 in systemic circulation were determined by generally accepted methods (for example as described in Evans, G., A Handbook of Bioanalysis and Drug Metabolism. Boca Raton, CRC Press (2004)). Specifically, liquid scintillation/mass spectrometry/mass spectrometry (LC/MS/MS) analytical methods were used to quantitate plasma concentrations of ILY-4001 after oral and intravenous administration. Pharmacokinetic parameters that were measured include Cₘₐₓ, AUC, tₘₐₓ, t_{1/2}, and F (bioavailability).

In this procedure, ILY-4001 was dosed at 3 mg/kg IV and 30 mg/kg oral. The results of this study, summarized in Table 2, showed a measured bioavailability of 28% of the original oral dose. This indicated about a 72% level of non-absorption of ILY-4001 from the GI tract into systemic circulation.

TABLE 2
Results of Pharmokinetic Study for ILY-4001

| | IV | ORAL |
|---|---|---|
| t½ (h) | 1.03 | 1.25 |
| Cmax (ng/mL) | 3168 | 2287 |
| Tmax (h) | 0.083 | 1 |
| AUC 0-24) (h*ng/mL) | 2793 | 5947 |
| AUC(0-inf) (h*ng/mL) | 2757 | 5726 |
| % F | | 28.0 |

### Example 1C

### CHARGE MODIFICATION OF ILY-4001 TO IMPROVE LUMEN-LOCALIZATION: SYNTHESIS OF 3-(3-AMINOOXALYL-1-BIPHENYL-2-YL METHYL-4-CARBOXYMETHOXY-2-METHYL-1H-INDOL-5-YL)-PROPIONIC ACID

This example describes an approach for charge modification of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam, to improve lumen-localization thereof. Specifically, ILY-4001 can be modified at certain substituent groups, including for example to change the ionic charge, and to impart improved lumen-localization. In this example, a scheme is presented by which ILY-4001 can be modified to add a propanoic acid moiety at position 5 (as shown in FIG. 5) to form 3-(3-aminooxalyl-1-biphenyl-2-yl methyl-4-carboxymethoxy-2-methyl-1H-indol-5-yl)-propionic acid.

Reference is made to FIG. 10, which outlines the overall synthesis scheme to prepare 3-(3-aminooxalyl-1-biphenyl-2-yl methyl-4-carboxymethoxy-2-methyl-1H-indol-5-yl)-propionic acid. The numbers under each compound shown in FIG. 10 correspond to the numbers in parenthesis associated with the chemical name for each compound in the following experimental description. The starting compound as shown in FIG. 10 (indicated with parenthetical (7)) can be prepared as shown in FIG. 7 and described in connection with Example 1A.

A solution of 1.0 g (4 mmol) of 7 in 10 mL of THF and 75 mL of DMF is stirred with 200 mg of NaH (60% in mineral oil; 5 mmol) for 10 min, and then with 0.4 mL (4.6 mmol) of allyl bromide for 2 h. The solution is diluted with water and extracted with EtOAc. The organic phase is washed with brine, dried over Na₂SO₄, evaporated at reduced pressure, and purified by column chromatography to obtain compound 10. This material is heated at reflux in 20 mL of N,N-dimethylaniline for 19 h, cooled, diluted with EtOAc, washed with 1 N HCl, H₂O, and brine, dried (Na₂SO₄), concentrated, and purified by column chromatography to obtain compound 11. This material (3.4 mmol) is dissolved in 60 mL of DMF and 10 mL of THF, 150 mg of NaH (60% in mineral oil; 3.7 mmol) is added, the mixture is stirred for 15 min, 0.4 mL (3.6 mmol) of ethyl bromoacetate is added, and stirring is continued for an additional 2.5 h. The solution is diluted with water and extracted with EtOAc. The organic phase is washed with brine, dried (Na₂SO₄), evaporated at reduced pressure, and purified by column chromatography to obtain compound 12. To a solution of 12 (0.022 mmol) in anhyd THF (1 mL) at r.t. is added BH3.THF (0.44 mL) complex (2.0 equiv, 1 M solution in THF, 0.044 mmol). The reaction mixture is stirred for 2 h at r.t., and is quenched carefully with drop wise addition of excess of 30% aq hydrogen peroxide and 15% aq NaOH. The mixture is then stirred vigorously for 30 min at r.t. The resultant mixture is was extracted, evaporated, and purified by column chromatography. The obtained alcohol in THF is added dropwisely to PCC solution and stirred for 3 hours. The reaction mixture is then purified to obtain compound 13. To a stirred solution of oxalyl chloride (1.2 mmol) in anhydrous CH₂Cl₂ (4 mL), a solution of compound 13 in CH₂Cl₂ (4 mL) is added drop wise and the reaction mixture is stirred for 80 min at RT. After cooling the reaction mixture to −10° C., a saturated solution of NH₃ in CH₂Cl₂ (10 mL) is added drop wise and then the reaction mixture is saturated with NH₃ (gas) at ca. 0° C. The reaction mixture is allowed to warm up to RT and is concentrated under reduced pressure to dryness and purified by column chromatography to obtain compound 14. To a stirred solution of compound 14 (1 mmol) in a mixture of THF (5 mL) and water (1 mL), a solution of lithium hydroxide monohydrate (2 mmol) in water (2 mL) is added portion wise and the reaction mixture is stirred for 2 h at RT. After addition of water (7 mL), the reaction mixture is concentrated under reduced pressure to a volume of ca. 100 mL. Then, to the residual yellow slurry, 2N HCl (2 mL) and EtOAc (20 mL) is added, the resultant mixture is stirred for 24 h at RT, and followed by column chromatography to obtain compound 15.

### Example 1D

### SYNTHESIS OF POLYMER-LINKED ILY-4001 TO IMPROVE LUMEN-LOCALIZATION: SYNTHESIS OF RANDOM COPOLYMER OF [3-AMINOOXALYL-2-METHYL-1-(2′-VINYL-BIPHENYL-2-YLMETHYL)-1H-INDOL-4-YLOXY]-ACETIC ACID, STYRENE, AND STYRENE SULFONIC ACID SODIUM SALT

This example describes approaches for synthesizing a phospholipase inhibitor comprising an oligomer or polymer moiety covalently linked to ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam, to improve lumen-localization thereof. Specifically, ILY-4001 was polymer linked to impart improved lumen-localization. In this example, a scheme is presented by which ILY-4001 can be linked to a random co-polymer to form to form a random copolymer of [3-Aminooxalyl-2-methyl-1-(2′-vinyl-biphenyl-2-ylmethyl)-1H-indol-4-yloxy]-acetic acid, styrene, and styrene sulfonic acid sodium salt.

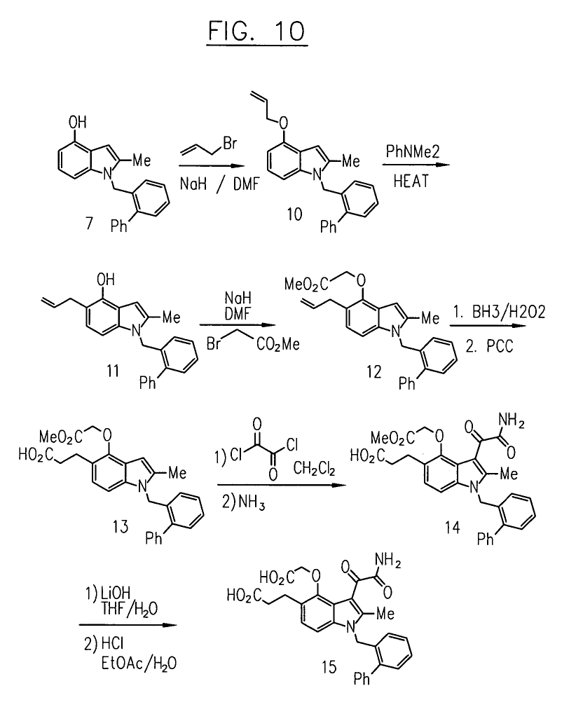

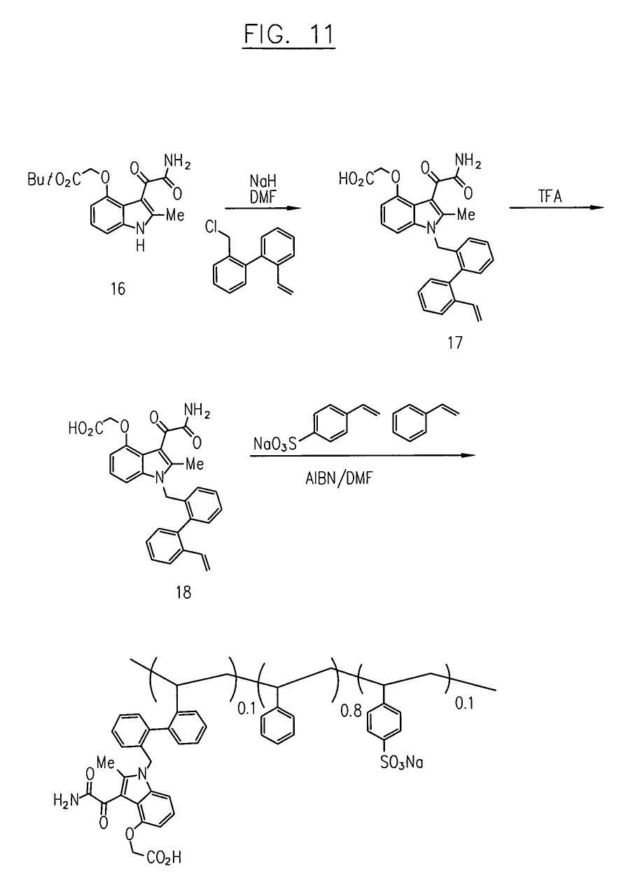

Referring to FIG. 11, the overall synthesis scheme for is outlined for polymer-linked ILY-4001. The numbers under each compound shown in FIG. 11 correspond to the numbers in parenthesis associated with the chemical name for each compound in the following experimental description. The starting compound as shown in FIG. 11 (indicated with parenthetical (16)) can be obtained from literature.

Compound 16 obtained from literature procedure (Bioorg. Med. Chem., 2004, 12, 1737-1749.) (0.10 mol) in anhydrous DMF (100 mL) is added drop wise to a stirred cooled (ca. 15° C.) suspension of sodium hydride (0.15 mol, 6.0 g, 60% in mineral oil, washed with 100 mL of hexanes before the reaction) in DMF (50 mL) and the reaction mixture is stirred for 0.5 h at RT. After cooling the reaction mixture to ca. 5° C., 2-(2-vinyl phenyl)benzyl chloride (0.101 mol) is added drop wise and the reaction mixture is stirred for 18 h at RT. The reaction is quenched with water (10 mL) and EtOAc (500 mL) is added. The resulted mixture is washed with water, brine, and dried over MgSO₄. After filtration and removal of the solvent from the filtrate under reduced pressure, the residue is purified by dry chromatography to afford product 17. To the solution of (1 mmol) of 17 in 15 mL of CH₂Cl₂ is added 2 mL of trifluoroacetic acid. This mixture is stirred for 1.5 hour, the solvent is evaporated at reduced pressure, and the residue is diluted with EtOAc and water. The organic phase is washed with brine, dried over MgSO₄, evaporated at reduced pressure, and purified by column chromatography to obtain compound 18. A mixture of 18, styrene sulfonic acid sodium salt, and styrene in mole ratio of 1:1:8 (in total one mmol) is dissolved in 2 mL of a mixed solvent (water/DMF=2/8 v/v). To the mixture AIBN (2,2′-azobisisobutyronitrile, 0.01 mmol) is added. The resulted solution is heated to 75° C. for 16 hours. After the reaction is cooled to rt, it is precipitated into iso-propyl alcohol twice, and dried under reduced pressure to obtain the co-polymer.

### Example 2

### LINKING TO INHIBITOR MOIETIES: SYNTHESIS OF [3-AMINOOXALYL-2-METHYL-1-(4-VINYL-BENZYL)-1H-INDOL-4-YLOXY]-ACETIC ACID (21); SYNTHESIS OF (1-ACRYLOYL-3-AMINOOXALYL-2-METHYL-1H-INDOL-4-YLOXY)-ACETIC ACID (23); SYNTHESIS OF {3-AMINOOXALYL-2-METHYL-1-[2-(PYRAZOLE-1-CARBOTHIOYLSULFANYL) PROPIONYL]-1H-INDOL-4-YLOXY}-ACETIC ACID (26)

This example describes approaches for covalently linking a phospholipase inhibiting moiety to linking moieties.

ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam, can be linked to various linking moieties (as a first step in a process to form compounds having improved lumen-localization thereof). In this example, a scheme is presented by which ILY-4001 can be provided with linking groups to form [3-Aminooxalyl-2-methyl-1-(4-vinyl-benzyl)-1H-indol-4-yloxy]-acetic acid (21); Synthesis of (1-Acryloyl-3-aminooxalyl-2-methyl-1H-indol-4-yloxy)-acetic acid (23); Synthesis of {3-Aminooxalyl-2-methyl-1-[2-(pyrazole-1-carbothioylsulfanyl)propionyl]-1H-indol-4-yloxy}-acetic acid (26).

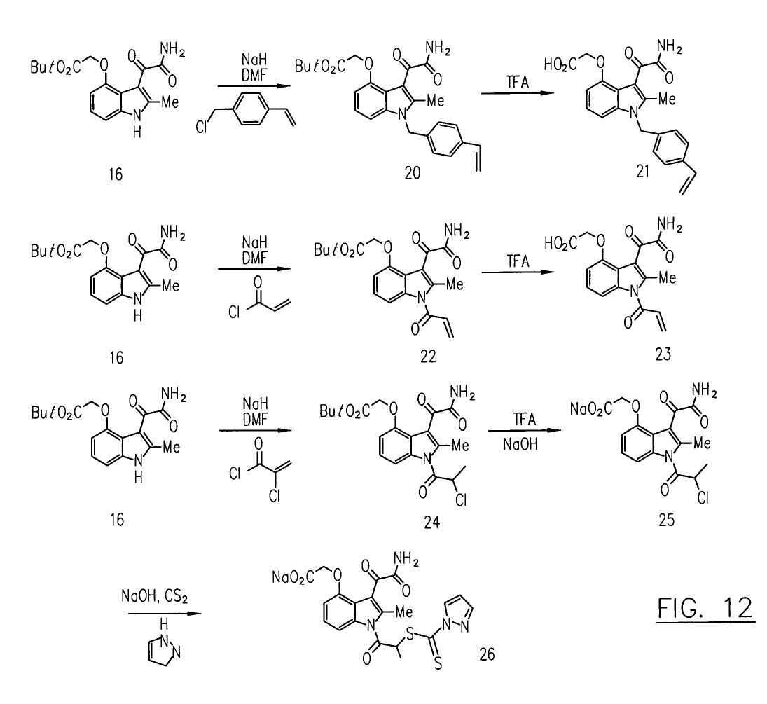

Referring to FIG. 12, the overall synthesis scheme for is outlined for preparing ILY-4001 with various linking groups. The numbers under each compound shown in FIG. 12 correspond to the numbers in parenthesis associated with the chemical name for each compound in the following experimental description. The starting compound as shown in FIG. 12 (indicated with parenthetical (16)) can be obtained from literature.

Compound 16 (0.10 mol) in anhydrous DMF (100 mL) is added drop wise to a stirred cooled (ca. 15° C.) suspension of sodium hydride (0.15 mol, 6.0 g, 60% in mineral oil, washed with 100 mL of hexanes before the reaction) in DMF (50 mL) and the reaction mixture is stirred for 0.5 h at RT. After cooling the reaction mixture to ca. 5° C., 4-vinyl benzyl chloride (0.101 mol) is added drop wise and the reaction mixture is stirred for 18 h at RT. The reaction is quenched with water (10 mL) and EtOAc (500 mL) is added. The resulted mixture is washed with water, brine, and dried over MgSO₄. After filtration and removal of the solvent from the filtrate under reduced pressure, the residue is purified by dry chromatography to afford product 20. To the solution of (1 mmol) of 20 in 15 mL of CH₂Cl₂ is added 2 mL of trifluoroacetic acid. This mixture is stirred for 1.5 hour, the solvent is evaporated at reduced pressure, and the residue is diluted with EtOAc and water. The organic phase is washed with brine, dried over MgSO₄, evaporated at reduced pressure, and purified by column chromatography to obtain compound 21.

A similar procedure is used to prepare compound 23.

A 100 mL round-bottomed flask equipped with a magnetic stirring bar and a PE stopper is charged with pyrazole (3 mmol), sodium hydroxide (0.12 g) and DMSO (5 mL) at ambient temperature (25° C.). Carbon disulfide (0.180 mL) is added to the flask dropwise. The mixture is further stirred for one hour. Compound 25 in DMSO obtained from the similar preceding procedure after treated with NaOH solution is then added to the reaction mixture slowly. The reaction is stirred for 2 hours. The solution is poured into 100 mL water is extracted with ethyl acetate. The organic layer is further washed with water (2×100 mL) and dried over MgSO₄. The solvent is removed under reduced pressure and the product is further purified by flash column chromatography.

### Example 3

### SYNTHESIS OF POLYMER-LINKED INHIBITORS

This example describes approaches for preparing polymer-linked inhibitors comprising an oligomer or polymer moiety covalently linked to an inhibiting moiety, where the polymer moiety is a soluble random co-polymer (Example 3A), or an insoluble cross-linked random copolymer (Example 3B).

### Example 3A

### SYNTHESIS OF POLYMER-LINKED INHIBITORS WITH SOLUBLE RANDOM COPOLYMER: SYNTHESIS OF COPOLYMER OF (1-ACRYLOYL-3-AMINOOXALYL-2-METHYL-1H-INDOL-4-YLOXY)-ACETIC ACID (23) AND DIMETHYL ACRYLAMIDE

In this example, approaches are outlined for synthesizing a phospholipase inhibitor comprising an oligomer or polymer moiety covalently linked to an inhibiting moiety, where the polymer moiety is a soluble random co-polymer. Specifically, a scheme is provided for synthesizing a copolymer of (1-Acryloyl-3-aminooxalyl-2-methyl-1H-indol-4-yloxy)-acetic acid (23) and dimethyl acrylamide.

A starting compound for this example can be from compound 23 having a linking group prepared as described in connection with Example 2. The polymer formed can be represented by the schematic chemical formula: 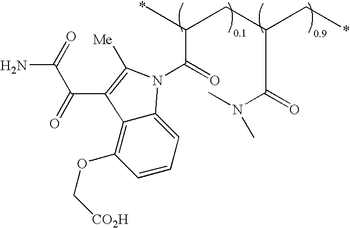
Briefly, a mixture of 23 and dimethyl acrylamide in mole ratio of 1:9 (in total one mmol) is dissolved in 2 mL of isopropanol. To the mixture AIBN (2,2′-azobisisobutyronitrile 0.01 mmol) is added. The resulted solution is heated to 75° C. for 8 hours. After the reaction is cooled to rt, it is diluted with 100 mL of water and dialyzed against water for 48 hours. The solution then is freeze-dried to obtain the co-polymer.

### Example 3B

### SYNTHESIS OF POLYMER-LINKED INHIBITORS WITH INSOLUBLE (CROSS-LINKED) RANDOM COPOLYMER: SYNTHESIS OF RANDOM COPOLYMER OF [3-AMINOOXALYL-2-METHYL-1-(4-VINYL-BENZYL)-1H-INDOL-4-YLOXY]-ACETIC ACID (21), STYRENE, AND STYRENE SULFONIC ACID SODIUM SALT, CROSSLINKED WITH DIVINYL BENZENE

This example describes approaches for synthesizing a phospholipase inhibitor comprising an oligomer or polymer moiety covalently linked to an inhibiting moiety, where the polymer moiety is an insoluble, cross-linked random co-polymer. Specifically, a scheme is provided for synthesizing a copolymer of [3-Aminooxalyl-2-methyl-1-(4-vinyl-benzyl)-1H-indol-4-yloxy]-acetic acid (21), styrene, and styrene sulfonic acid sodium salt, crosslinked with divinyl benzene.

A starting compound for this example can be from compound 21 having a linking group prepared as described in connection with Example 2. The polymer formed can be represented by the schematic chemical formula:

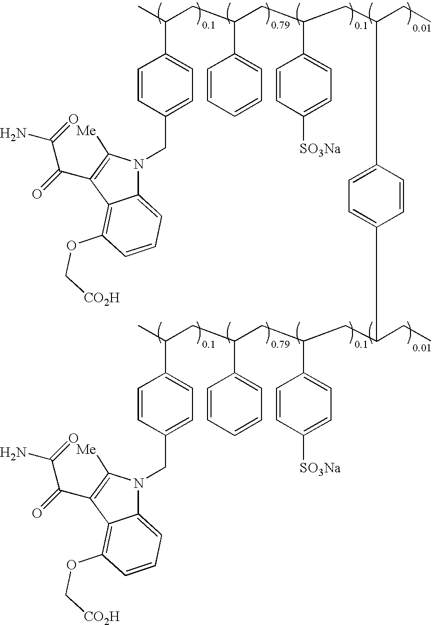

A mixture of 21, styrene sulfonic acid sodium salt, styrene, divinyl benzene in mole ratio of 1:1:7.9:0.1 (in total 10 mmol) is dissolved in 20 mL of a mixed solvent (water/DMF=2/8 v/v). To the mixture AIBN (2,2′-azobisisobutyronitrile 0.1 mmol) is added. The resulted solution is heated to 75° C. for 24 hours. After the reaction is cooled to rt, the resulted crosslinked solid material is mechanically milled into find gel, washed with excess amount of water, dried under reduced pressure to obtain the co-polymer.

### Example 4

### SYNTHESIS OF POLYMER-LINKED INHIBITORS BY POLYMER-PARTICLE MODIFICATION: SYNTHESIS OF (3-AMINOOXALYL-1-DODECYL-2-METHYL-1H-INDOL-4-YLOXY)-ACETIC ACID MODIFIED CAVILINK™ BEAD

This example describes approaches for synthesizing a phospholipase inhibitor comprising an oligomer or polymer moiety covalently linked to an inhibiting moiety, where the polymer moiety is an insoluble particle, and the inhibiting moiety is linked to the particle. Specifically, a scheme is provided for synthesis of (3-Aminooxalyl-1-dodecyl-2-methyl-1H-indol-4-yloxy)-acetic acid modified Cavilink™ bead.

The polymer formed can be represented by the schematic representation: 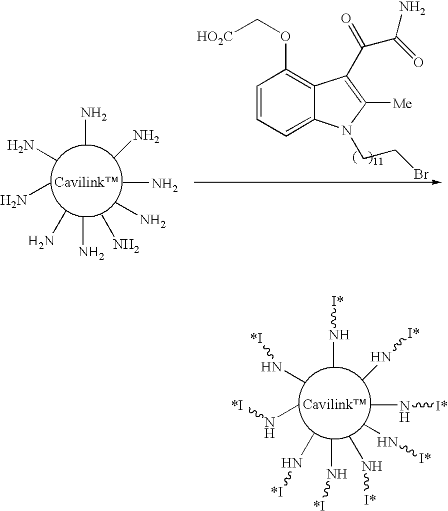
Commercial available polystyrene Cavilink™ Bead (1 g) is suspended in ethanol at rt. To the solution, the inhibitor compound (100 mg) (shown above the arrow as a reactant; represented as “I” in the product compound) is added and stirred for 24 hours. The bead is filtered and washed with excess of ethanol until no detection of inhibitor by UV. The bead then is dried under reduced pressure.

### Example 5

### SYNTHESIS OF POLYMER-LINKED INHIBITORS WITH GRAFT COPOLYMERS: SYNTHESIS OF STAR COPOLYMER OF (1-ACRYLOYL-3-AMINOOXALYL-2-METHYL-1H-INDOL-4-YLOXY)-ACETIC ACID, N-BUTYL ACRYLATE, DIMETHYL ACRYLAMIDE, AND N-(2-ACRYLOYLAMINO-ETHYL)-ACRYLAMIDE

This example describes approaches for synthesizing a phospholipase inhibitor comprising an oligomer or polymer moiety covalently linked to an inhibiting moiety, where the polymer moiety is linked using graft copolymers. In particular, a scheme is provided for synthesis of a star copolymer of (1-Acryloyl-3-aminooxalyl-2-methyl-1H-indol-4-yloxy)-acetic acid, n-butyl acrylate, dimethyl acrylamide, and N-(2-Acryloylamino-ethyl)-acrylamide.

The synthesis scheme and the polymer formed thereby can be represented by the schematic representation: 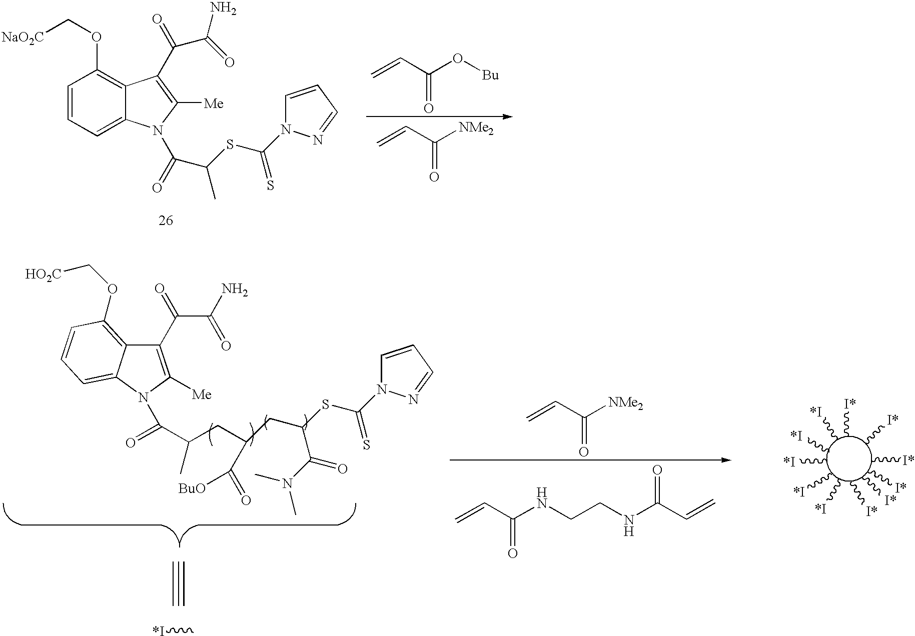
A mixture of 26, dimethyl acrylamide, and n-butyl acrylate in a mole ratio of 0.04:0.48:0.48 (in total 10 mmol) is dissolved in 20 mL of DMF. To the mixture AIBN (2,2′-azobisisobutyronitrile, 10 mmol % to compound 26) is added and is heated to 75° C. for 8 hours. To the resulted yellow solution 1 mmol of dimethyl acrylamide and ethylene bis-diacrylamide (1:1) is added and stirred for an additional 8 hours. After the reaction is cooled to rt, the reaction mixture is precipitated twice, dried under reduced pressure to obtain the co-polymer.

### Example 6A

### SYNTHESIS OF TAILORED-POLYMER-SINGLET: SYNTHESIS OF POLY-N-BUTYL ACRYLATE TAILORED (1-ACRYLOYL-3-AMINOOXALYL-2-METHYL-1H-INDOL-4-YLOXY)-ACETIC ACID

This example describes approaches for synthesizing a phospholipase inhibitor comprising an oligomer or polymer moiety covalently linked to a single inhibiting moiety to form a phospholipase inhibitor “singlet”. Specifically, a scheme is provided for synthesis of poly-n-butyl acrylate tailored (1-Acryloyl-3-aminooxalyl-2-methyl-1H-indol-4-yloxy)-acetic acid.

The synthesis scheme and the polymer formed thereby can be represented by the schematic representation: 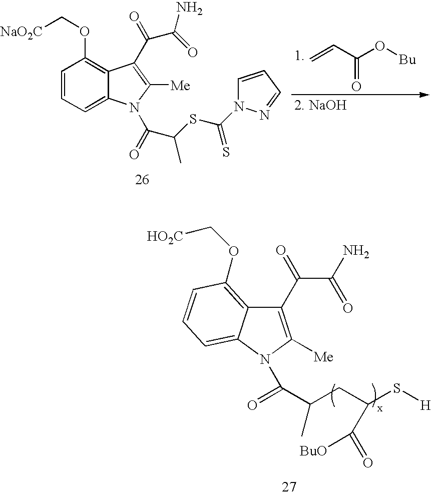
A mixture of 26 and n-butyl acrylate in a mole ratio of 0.04:0.96 (in total 10 mmol) is dissolved in 20 mL of DMF. To the mixture AIBN (2,2′-azobisisobutyronitrile, 10 mmol % to compound 26) is added and is heated to 75 for 16 hours. After the reaction is cooled to 45° C., to the resulted yellow solution 2 mL of 10% NaOH solution is added and stirred for an additional 8 hours. After the reaction is cooled to rt, the reaction mixture is precipitated twice, dried under reduced pressure to obtain the co-polymer.

### Example 6B

### SYNTHESIS OF TAILORED-POLYMER-DIMERS

This example describes various approaches for synthesizing a phospholipase inhibitor comprising an oligomer or polymer moiety covalently linked to two inhibiting moieties to form a phospholipase inhibitor “dimer”. Specifically, in a first approach, a scheme for the synthesis of disulfide dimer of poly-n-butyl acrylate tailored (1-Acryloyl-3-aminooxalyl-2-methyl-1H-indol-4-yloxy)-acetic acid is disclosed (Example 6B-1). In a second approach, a scheme for the synthesis of (3-Aminooxalyl-1-{12-[12-(3-aminooxalyl-4-carboxymethoxy-2-methyl-indol-1-yl)-dodecyldisulfanyl]-dodecyl}-2-methyl-1H-indol-4-yloxy)-acetic acid (31).

### Example 6B-1

### SYNTHESIS OF TAILORED-POLYMER-DIMER: SYNTHESIS OF DISULFIDE DIMER OF POLY-N-BUTYL ACRYLATE TAILORED (1-ACRYLOYL-3-AMINOOXALYL-2-METHYL-1H-INDOL-4-YLOXY)-ACETIC ACID

The synthesis scheme and the polymer formed thereby can be represented by the schematic representation: 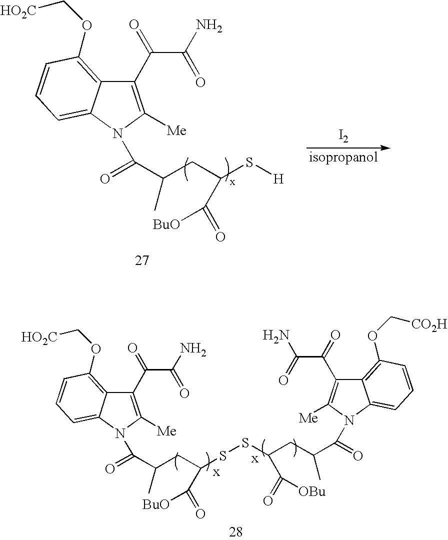
To a solution of 27 (1 mmol) in isopropanol (10 mL) is added iodine (127 mg, 0.5 mmol). After 2 hours, the reaction mixture is concentrated and redissolved in EtOAc (25 mL). the solution is washed with Na₂S₂O₄ (2×10 mL) and brine (10 mL), dried over sodium sulfate, filtered, and concentrated in vacuo. The product was purified by precipitation to provide disulfide 28

### Example 6B-2

### SYNTHESIS OF TAILORED-POLYMER-DIMER: SYNTHESIS OF (3-AMINOOXALYL-1-{12-[12-(3-AMINOOXALYL-4-CARBOXYMETHOXY-2-METHYL-INDOL-1-YL)-DODECYLDISULFANYL]-DODECYL}-2-METHYL-1H-INDOL-4-YLOXY)-ACETIC ACID (31)

The synthesis scheme and the polymer formed thereby can be represented by the schematic representation:

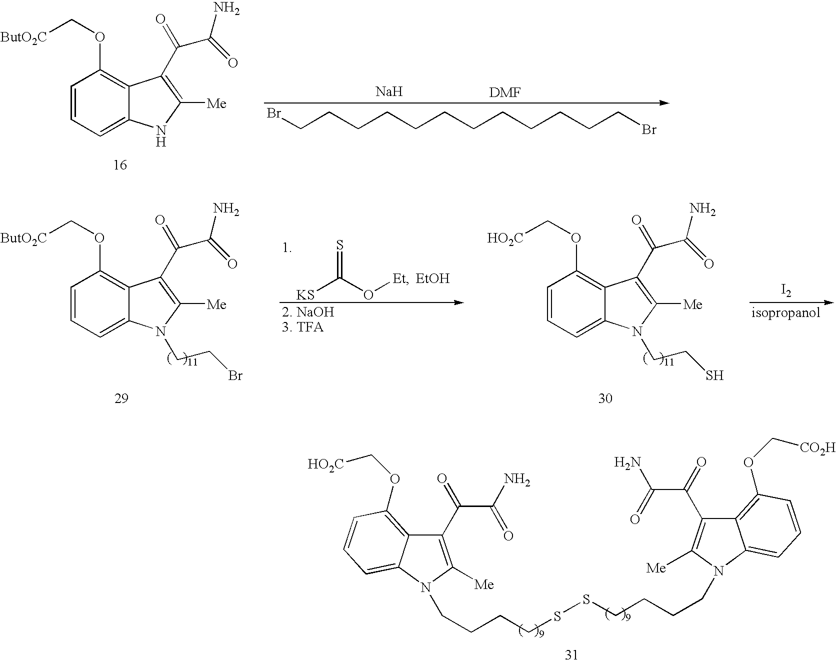

Compound 16 (10 mol) in anhydrous DMF (100 mL) is added drop wise to a stirred cooled (ca. 15° C.) suspension of sodium hydride (0.015 mol, 600 mg, 60% in mineral oil, washed with 10 mL of hexanes before the reaction) in DMF (50 mL) and the reaction mixture is stirred for 0.5 h at RT. After cooling the reaction mixture to ca. 5° C., 1,12-dibromododecane (10.1 mmol) is added at once and the reaction mixture is stirred for 18 h at RT. The reaction is quenched with water (10 mL) and EtOAc (500 mL) is added. The resulted mixture is washed with water, brine, and dried over MgSO₄. After filtration and removal of the solvent from the filtrate under reduced pressure, the residue is purified by dry chromatography to afford product 29. To the solution of (1 mmol) of 29 in 30 mL of EtOH is added 1.1 mmol of dithiocarbonic acid ethyl ester potassium salt. This mixture is stirred for 12 hour and then the reaction is heated to 45° C. To the resulted yellow solution 2 mL of 10% NaOH solution is added and stirred for an additional 8 hours. After the reaction is cooled to rt, solvent is removed and extracted with EtOAc. The resulted mixture is washed with water, brine, and dried over MgSO₄ to obtain a crude product. To the solution of (1 mmol) of the crude product in 15 mL of CH₂Cl₂ is added 2 mL of trifluoroacetic acid. This mixture is stirred for 1.5 hour, the solvent is evaporated at reduced pressure, and the residue is diluted with EtOAc and water. The organic phase is washed with brine, dried over MgSO₄, evaporated at reduced pressure, and purified by column chromatography to obtain compound 30. To a solution of 30 (1 mmol) in isopropanol (10 mL) is added iodine (127 mg, 0.5 mmol). After 2 hours, the reaction mixture is concentrated and redissolved in EtOAc (25 mL). the solution is washed with Na₂S₂O₄ (2×10 mL) and brine (10 mL), dried over sodium sulfate, filtered, and concentrated in vacuo. The product was purified by column chromatography to provide disulfide 31.

### Example 7

### REDUCTION IN INSULIN RESISTANCE IN A MOUSE MODEL

A phospholipase inhibitor, for example a composition comprising a phospholipase inhibiting moiety disclosed herein, can be used in a mouse model to demonstrate, for example, suppression of diet-induced insulin resistance, relating to, for example, diet-induced onset of diabetes. The phospholipase inhibitor can be administered to subject animals either as a chow supplement and/or by oral gavage BID in a certain dosage (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to about 90 mg/kg body weight.

The mouse chow used may have a composition representative of a Western (high fat and/or high cholesterol) diet. For example, the chow may contain about 21% milk fat and about 0.15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

The duration of the study is typically about 6 to about 8 weeks, with the subject animals being dosed every day during this period. Typical dosing groups, containing about 6 to about 8 animals per group, can be composed of an untreated control group, a vehicle control group, and dose-treated groups ranging from about 10 mg/kg body weight to about 90 mg/kg body weight.

At the end of the about 6 to about 8 week study period, an oral glucose tolerance test and/or an insulin sensitivity test can be conducted as follows:

Oral glucose tolerance test—after an overnight fast, mice from each dosing group can be fed a glucose bolus (e.g., by stomach gavage using about 2 g/kg body weight) in about 50 μl of saline. Blood samples can be obtained from the tail vein before, and about 15, about 30, about 60, and about 120 minutes after glucose administration; blood glucose levels at the various time points can then be determined.

Insulin sensitivity test—after about a 6 hour morning fast, mice in each of the dosing groups can be administered bovine insulin (e.g., about 1 U/kg body weight, using, e.g., intraperitoneal administration. Blood samples can be obtained from the tail vein before, and about 15, about 30, about 60, and about 120 minutes after insulin administration; plasma insulin levels at the various time points can then be determined, e.g., by radioimmunoassay.

The effect of the non-absorbed phospholipase inhibitor, e.g., a phospholipase A2 inhibitor, is a decrease in insulin resistance, i.e., better tolerance to glucose challenge by, for example, increasing the efficiency of glucose metabolism in cells, and in the animals of the dose-treated groups fed a Western (high fat/high cholesterol) diet relative to the animals of the control groups. Effective dosages can also be determined.

### Example 8

### REDUCTION IN FAT ABSORPTION IN A MOUSE MODEL

A phospholipase inhibitor, for example a composition comprising a phospholipase inhibiting moiety disclosed herein, can be used in a mouse model to demonstrate, for example, reduced lipid absorption in subjects on a Western diet. The phospholipase inhibitor can be administered to subject animals either as a chow supplement and/or by oral gavage BID in a certain dosage (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to 90 mg/kg body weight.

The mouse chow used may have a composition representative of a Western-type (high fat and/or high cholesterol) diet. For example, the chow may contain about 21% milk fat and about 0.15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

Triglyceride measurements can be taken for a duration of about 6 to about 8 weeks, with the subject animals being dosed every day during this period. Typical dosing groups, containing about 6 to about 8 animals per group, can be composed of an untreated control group, a vehicle control group, and dose-treated groups ranging from about 10 mg/kg body weight to about 90 mg/kg body weight. On a weekly basis, plasma samples can be obtained from the subject animals and analyzed for total triglycerides, for example, to determine the amount of lipids absorbed into the blood circulation.

The effect of the non-absorbed phospholipase inhibitor, e.g., a phospholipase A2 inhibitor, is a net decrease in lipid plasma levels, which indicates reduced fat absorption, in the animals of the dose-treated groups fed a Western (high fat/high cholesterol) diet relative to the animals of the control groups. Effective dosages can also be determined.

### Example 9

### REDUCTION IN DIET-INDUCED HYPERCHOLESTEROLEMIA IN A MOUSE MODEL

A phospholipase inhibitor, for example a composition comprising a phospholipase inhibiting moiety disclosed herein, can be used in a mouse model to demonstrate, for example, suppression of diet-induced hypercholesterolemia. The phospholipase inhibitor can be administered to subject animals either as a chow supplement and/or by oral gavage BID (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to about 90 mg/kg body weight.

The mouse chow used may have a composition representative of a Western-type (high fat and/or high cholesterol) diet. For example, the chow may contain about 21% milk fat and about 0.15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

Cholesterol and/or triglyceride measurements can be taken for a duration of about 6 to about 8 weeks, with the subject animals being dosed every day during this period. Typical dosing groups, containing about 6 to about 8 animals per group, can be composed of a untreated control group, a vehicle control group, and dose-treated groups ranging from about 10 mg/kg body weight to about 90 mg/kg body weight. On a weekly basis, plasma samples can be obtained from the subject animals and analyzed for total cholesterol and/or triglycerides, for example, to determine the amount of cholesterol and/or lipids absorbed into the blood circulation. Since most plasma cholesterol in a mouse is associated with HDL (in contrast to the LDL association of most cholesterol in humans), HDL and non-HDL fractions can be separated to aid determination of the effectiveness of the non-absorbed phospholipase inhibitor in lowering plasma non-HDL levels, for example VLDL/LDL.

The effect of the non-absorbed phospholipase inhibitor, e.g., a phospholipase A2 inhibitor, is a net decrease in hypercholesterolemia in the animals of the dose-treated groups fed a Western (high fat/high cholesterol) diet relative to the animals of the control groups. Effective dosages can also be determined.

### Example 10

### IN-VIVO EVALUATION OF ILY-4001 [2-(3-(2-AMINO-2-OXOACETYL)-1-(BIPHENYL-2-YLMETHYL)-2-METHYL-1H-INDOL-4-YLOXY)ACETIC ACID] AS PLA2-IB INHIBITOR AND FOR TREATMENT OF DIET-RELATED CONDITIONS

This example demonstrated that the compound 2-(3-(2-amino-2-oxoacetyl)-1-(biphenyl-2-ylmethyl)-2-methyl-1H-indol-4-yloxy)acetic acid, shown in FIG. 7, was an effective phospholipase-2A I inhibitor, with phenotypic effects approaching and/or comparable to the effect of genetically deficient PLA2 (−/−) mice. This example also demonstrated that this compound is effective in treating conditions such as weight-related conditions, insulin-related conditions, and cholesterol-related conditions, including in particular conditions such as obesity, diabetes mellitus, insulin resistance, glucose intolerance, hypercholesterolemia and hypertriglyceridemia. In this example, the compound 2-(3-(2-amino-2-oxoacetyl)-1-(biphenyl-2-ylmethyl)-2-methyl-1H-indol-4-yloxy)acetic acid is designated as ILY-4001 (and is alternatively referred to herein as methyl indoxam).

ILY-4001 (FIG. 7) was evaluated as a PLA2 IB inhibitor in a set of experiments using wild-type mice and genetically deficient PLA2 (−/−) mice (also referred to herein as PLA2 knock-out (KO) mice). In these experiments, wild-type and PLA2 (−/−) mice were maintained on a high fat/high sucrose diet, details of which are described below.

ILY-4001 has a measured IC50 value of around 0.2 uM versus the human PLA2 IB enzyme and 0.15 uM versus the mouse PLA2 IB enzyme, in the context of the 1-palmitoyl-2-(10-pyrenedecanoyl)-sn-glycero-3-phosphoglycerol assay, which measures pyrene substrate release from vesicles treated with PLA2 IB enzyme (Singer, Ghomashchi et al. 2002). An IC-50 value of around 0.062 was determined in experimental studies. (See Example 1B-1). In addition to its activity against mouse and human pancreatic PLA2, methyl indoxam is stable at low pH, and as such, would be predicted to survive passage through the stomach. ILY-4001 has relatively low absorbtion from the GI lumen, based on Caco-2 assays (See Example 1B-2), and based on pharmokinetic studies (See Example 1B-3).

In the study of this Example 10, twenty-four mice were studied using treatment groups as shown in Table 3, below. Briefly, four groups were set up, each having six mice. Three of the groups included six wild-type PLA2 (+/+) mice in each group (eighteen mice total), and one of the groups included six genetically deficient PLA2 (−/−) mice. One of the wild-type groups was used as a wild-type control group, and was not treated with ILY-4001. The other two wild-type groups were treated with ILY-4001—one group at a lower dose (indicated as “L” in Table 1) of 25 mg/kg/day, and the other at a higher dose (indicated as “H” in Table 1) of 90 mg/kg/day. The group comprising the PLA2 (−/−) mice was used as a positive control group.

TABLE 3
Treatment Groups for ILY-4001 Study

| | | Number | ILY-4001 Dose | |
|---|---|---|---|---|
| Group | | of | Levels | Duration |
| Number | Treatment Groups | Animals | (mg/kg/day) | (weeks) |
| 1 | C57BL/6(wt) | 6 | 0 | 10 |
| 2 | C57BL/6(wt) | 6 | 25 (L) | 10 |
| 3 | C57BL/6(wt) | 6 | 90 (H) | 10 |
| 4 | C57BL/6(PLA₂-KO) | 6 | 0 | 10 |

The experimental protocol used in this study was as follows. The four groups of mice, including wild type and isogenic PLA2 (−/−) C57BL/J mice were acclimated for three days on a low fat/low carbohydrate diet. After the three day acclimation phase, the animals were fasted overnight and serum samples taken to establish baseline plasma cholesterol, triglyceride, and glucose levels, along with baseline body weight. The mice in each of the treatment groups were then fed a high fat/high sucrose diabetogenic diet (Research Diets D12331). 1000 g of the high fat/high sucrose D12331 diet was composed of casein (228 g), DL-methionine (2 g), maltodextrin 10 (170 g), sucrose (175 g), soybean oil (25 g), hydrogenated coconut oil (333.5 g), mineral mix S10001 (40 g), sodium bicarbonate (10.5 g), potassium citrate (4 g), vitamin mix V10001 (10 g), and choline bitartrate (2 g). This diet was supplemented with ILY-4001 treatments such that the average daily dose of the compound ingested by a 25 g mouse was: 0 mg/kg/day (wild-type control group and PLA2 (−/−) control group); 25 mg/kg/day (low-dose wild-type treatment group), or 90 mg/kg/day (high-dose wild-type treatment group). The animals were maintained on the high fat/high sucrose diet, with the designated ILY-4001 supplementation, for a period of ten weeks.

Body weight measurements were taken for all animals in all treatment and control groups at the beginning of the treatment period and at 4 weeks and 10 weeks after initiation of the study. (See Example 10A). Blood draws were also taken at the beginning of the treatment period (baseline) and at 4 weeks and 10 weeks after initiation of the study, in order to determine fasting glucose (See Example 10B). Cholesterol and triglyceride levels were determined from blood draws taken at the beginning of the treatment (baseline) and at ten weeks. (See Example 1° C.).

### Example 10A

### BODY-WEIGHT GAIN IN IN-VIVO EVALUATION OF ILY-4001 [2-(3-(2-AMINO-2-OXOACETYL)-1-(BIPHENYL-2-YLMETHYL)-2-METHYL-1H-INDOL-4-YLOXY)ACETIC ACID] AS PLA2-IB INHIBITOR

In the study generally described above in Example 10, body weight measurements were taken for all animals in all treatment and control groups at the beginning of the treatment period and at 4 weeks and 10 weeks after initiation of the study. Using the treatment protocol described above with ILY-4001 supplemented into a high fat/high sucrose diabetogenic diet, notable decreases were seen in body weight gain.

With reference to FIG. 13A, body weight gain in the wild-type mice receiving no ILY-4001 (group 1, wild-type control) followed the anticipated pattern of a substantial weight gain from the beginning of the study to week 4, and a further doubling of weight gain by week 10. In contrast, body weight gain for the PLA2 (−/−) mice (PLA2 KO mice) also receiving no ILY-4001 and placed on the same diet (group 4, PLA2 (−/−) control) did not show statistically significant changes from week 4 to week 10, and only a marginal increase in body weight over the extent of the study (<5 g). The two treatment groups (25 mg/kg/d and 90 mg/kg/d) showed significantly reduced body weight gains at week 4 and week 10 of the study compared to the wild-type control group. Both treatment groups showed body weight gain at four weeks modulated to an extent approaching that achieved in the PLA2 (−/−) mice. The low-dose treatment group showed body weight gain at ten weeks modulated to an extent comparable to that achieved in the PLA2 (−/−) mice.

### Example 10B

### FASTING SERUM GLUCOSE IN IN-VIVO EVALUATION OF ILY-4001 [2-(3-(2-AMINO-2-OXOACETYL)-1-(BIPHENYL-2-YLMETHYL)-2-METHYL-1H-INDOL-4-YLOXY)ACETIC ACID] AS PLA2-IB INHIBITOR

In the study generally described above in Example 10, blood draws were taken at the beginning of the treatment period (baseline) and at 4 weeks and 10 weeks after initiation of the study, in order to determine fasting glucose. Using the treatment protocol described above with ILY-4001 supplemented into a high fat/high sucrose diabetogenic diet, notable decreases were seen in fasting serum glucose levels.

Referring to FIG. 13B, the wild-type control mice (group 1) showed a sustained elevated plasma glucose level, consistent with and indicative of the high fat/high sucrose diabetogenic diet at both four weeks and ten weeks. In contrast, the PLA2 (−/−) KO mice (group 4) showed a statistically significant decrease in fasting glucose levels at both week 4 and week 10, reflecting an increased sensitivity to insulin not normally seen in mice placed on this diabetogenic diet. The high dose ILY-4001 treatment group (group 3) showed a similar reduction in fasting glucose levels at both four weeks and ten weeks, indicating an improvement in insulin sensitivity for this group as compared to wild-type mice on the high fat/high sucrose diet, and approaching the phenotype seen in the PLA2 (−/−) KO mice. In the low dose ILY-4001 treatment group (group 2), a moderately beneficial effect was seen at week four; however, a beneficial effect was not observed at week ten.

### Example 10C

### SERUM CHOLESTEROL AND TRIGLYCERIDES IN IN-VIVO EVALUATION OF ILY-4001 [2-(3-(2-AMINO-2-OXOACETYL)-1-(BIPHENYL-2-YLMETHYL)-2-METHYL-1H-INDOL-4-YLOXY)ACETIC ACID] AS PLA2-IB INHIBITOR

In the study generally described above in Example 10, blood draws were taken at the beginning of the treatment period (baseline) and at 10 weeks after initiation of the study, in order to determine cholesterol and triglyceride levels. Using the treatment protocol described above with ILY-4001 supplemented into a high fat/high sucrose diabetogenic diet, notable decreases were seen in both serum cholesterol levels and serum triglyceride levels.

With reference to FIGS. 13C and 13D, after 10 weeks on the high fat/high sucrose diet, the wild-type control animals (group 1) had notable and substantial increases in both circulating cholesterol levels (FIG. 13C) and triglyceride levels (FIG. 13D), relative to the baseline measure taken at the beginning of the study. The PLA2 (−/−) KO animals (group 4), in contrast, did not show the same increase in these lipids, with cholesterol and triglyceride values each 2 to 3 times lower than those found in the wild-type control group. Significantly, treatment with ILY-4001 at both the low and high doses (groups 2 and 3, respectively) substantially reduced the plasma levels of cholesterol and triglycerides, mimicking the beneficial effects at levels comparable to the PLA2 (−/−) KO mice.

### Example 11

### SYNTHESIS OF MULTIVALENT INDOLE AND INDOLE RELATED COMPOUNDS

This example shows the preparation of multivalent indole or indole-related compounds comprising two or more indole or indole-related moieties (e.g., phospholipase inhibiting moieties) each covalently linked to a multifunctional bridge moiety.

### Example 11.1

### (INTERMEDIATE) TERT-BUTYL 2-(3-(2-AMINO-2-OXOACETYL)-1-(8-BROMOOCTYL)-2-METHYL-1H-INDOL-4-YLOXY)ACETATE

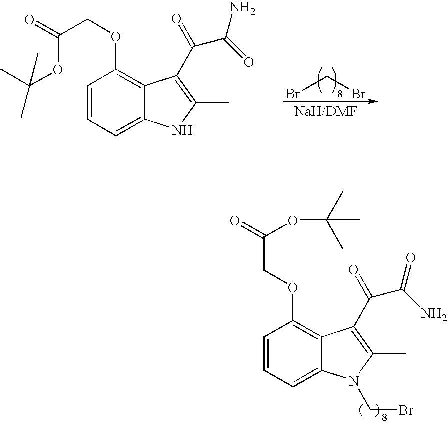

tert-Butyl 2-(3-(2-amino-2-oxoacetyl)-1-(8-bromooctyl)-2-methyl-1H-indol-4-yloxy)acetate was prepared as follows, as a starting material for later examples:

A solution of the starting indole (3.3 g, 10 mmol) in 10 mL of anhydrous DMF was cooled in an ice bath and dry sodium hydride (290 mg, 12 mmol, 1.2 equiv) was added. After stirring under nitrogen for 30 min at 0° C., the mixture was transferred dropwise into a solution of 1,8-dibromooctane (2.2 mL, 3.3 g, 12 mmol, 1.2 equiv) in 5 mL of anhydrous DMF also cooled in an ice bath. The resulting orange mixture was stirred under nitrogen for 4 h at 0° C., and it was then allowed to warm to RT. After an overnight stirring at RT, the reaction mixture was quenched with 15 mL of NH₄Cl and concentrated under reduced pressure. It was then diluted with 100 mL of DCM, washed with NH₄Cl (40 mL) and twice with brine (2×40 mL), dried over MgSO₄ and concentrated in vacuo to afford the crude product as an orange oil. Purification by flash-chromatography (H/EA: 3/2, 1/1 then 2/3) yielded pure bromoalkyl (2.6 g, 50%) as a yellow solid.

¹H NMR (CD₃OD, 300 MHz): δ 7.10 (dd, 1H, J=9.0, 8.1 Hz, H-6), 7.08 (dd, 1H, J=8.1, 1.5 Hz, H-5), 6.44 (dd, 1H, J=9.0, 1.5 Hz, H-7), 4.63 (s, 2H, H-10), 4.17 (t, 2H, J=7.5 Hz, H-14), 3.41 (t, 2H, J=6.9 Hz, H-15), 2.60 (s, 3H, H-9), 1.80-1.75 (m, 4H, H-16+H-17), 1.44 (s, 9H, C(CH₃)₃), 1.41-1.33 (m, 8H, CH₂).

¹³C NMR (CD₃OD, 75.5 MHz): δ 188.8 (12), 170.2 (11), 169.2 (13), 152.0 (4), 145.2 (1), 138.0 (8), 123.1 (3), 116.7 (6), 110.1 (5), 104.1 (7+2), 82.1 (C(CH₃)₃), 65.6 (10), 43.3 (14), 33.2 (15), 32.7 (17), 29.4 (16), 29.0 (CH₂), 28.5 (CH₂), 27.8 (CH₂), 27.1 (C(CH₃)₃), 26.6 (CH₂), 10.7 (9).

MS (ESI, MeOH): m/z 545.2 [M+Na]⁺ (100%, ⁷⁹Br isotope), 547.2 [M+Na]⁺ (97%, ⁸¹Br isotope).

### Example 11.2

### (INTERMEDIATE) SYNTHESIS OF TERT-BUTYL 2-(3-(2-AMINO-2-OXOACETYL)-1-(12-BROMODODECYL)-2-METHYL-1H-INDOL-4-YLOXY)ACETATE

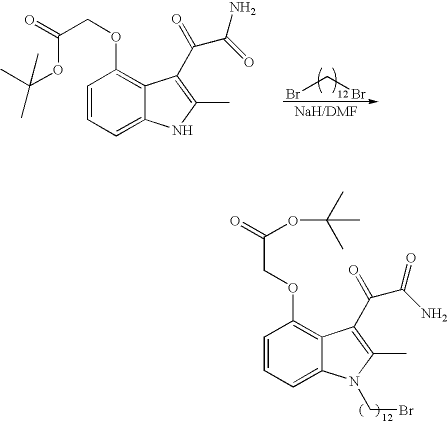

tert-Butyl 2-(3-(2-amino-2-oxoacetyl)-1-(12-bromododecyl)-2-methyl-1H-indol-4-yloxy)acetate was prepared as follows as a starting material for use in other examples.

The starting indole intermediate (2.54 g, 7.65 mmole) in dry DMF (10 mL), at 0° C. under nitrogen, had 95% sodium hydride (0.233 g, 9.22 mmole) added. The dark mixture was stirred at 0° C. for 0.5 h and then added dropwise over 10 minutes to a solution of 1,12-dibromododecane (4.5 g, 13.71 mmole) in dry DMF (20 mL) at 0° C. The mixture was stirred at 0° C. for 5 h and at room temperature for 19 h. The reaction was cooled to 0° C., quenched with ammonium chloride solution (10 mL), and diluted with dichloromethane (100 mL). The mixture was washed with ammonium chloride solution (50 mL) and the aqueous phase extracted with dichloromethane (4×25 mL). The combined organic phase was washed with brine (100 mL), dried (Na₂SO₄), filtered and evaporated to a red/brown liquid which was further evaporated under high vacuum. The residue was a thick red/brown semi-solid, which was purified by chromatography over silica gel, using chloroform/hexanes (8:1) as the eluant, gave the product as an orange/brown semi-solid (2.00 g, 45%).

### Example 11.3

### COMPOUND (5-27)

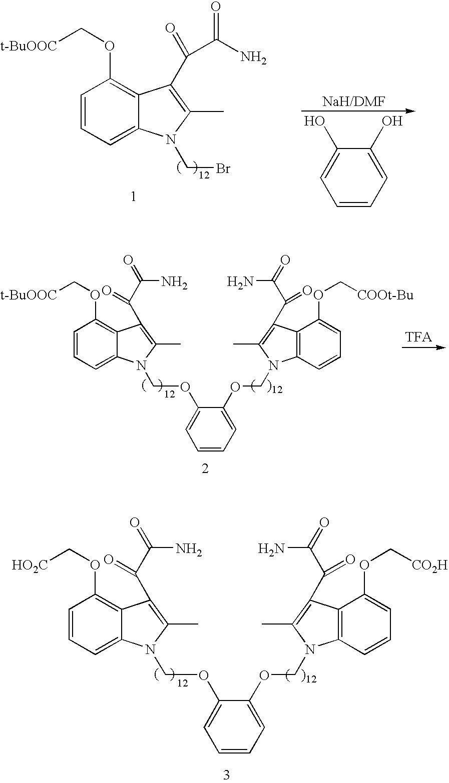

First, the t-Bu protected compound, [3-Aminooxalyl-1-(12-{2-[12-(3-aminooxalyl 4-tert-butoxycarbonylmethoxy-2-methyl-indol-1-yl)-dodecyloxy]-phenoxy}-dodecyl)-2-methyl-1H-indol-4-yloxy]-acetic acid tert-butyl ester, 2 was prepared as follows.

Catechol (0.054 g, 0.49 mmole) in dry DMF (6 mL), at 0° C. under nitrogen, had 95% sodium hydride (0.027 g, 1.08 mmole) added. The mixture was stirred at 0° C. for 0.5 h and then the bromide 1 (0.600 g, 1.03 mmole) (prepared as in Example 11B) in dry DMF (7 mL) was added over 3 minutes. The mixture was stirred at 0° C. for 8 h and slowly warmed to room temperature overnight. The mixture was cooled to 0° C., quenched with ammonium chloride solution (5 mL), diluted with dichloromethane (100 mL) and ammonium chloride (45 mL). The organic phase was separated and the aqueous phase extracted with dichloromethane (6×50 mL). The combined organic phase was evaporated to near dryness, dissolved in dichloromethane (100 mL) and washed with water (50 mL). The aqueous phase was extracted with dichloromethane (2×50 mL). The combined organic phase was dried (Na₂SO₄), filtered and evaporated to a red/brown semi-solid. Purification by chromatography over silica gel, using chloroform/hexanes (7:1 to 4:1) as the eluant, gave the product as an orange/brown semi-solid (0.029 g, 5%).

The diester 2 in above scheme was deprotected to form [3-Aminooxalyl-1-(12-{2-[12-(3-aminooxalyl-4-carboxymethoxy-2-methyl-indol-1-yl)-dodecyloxy]-phenoxy}-dodecyl)-2-methyl-1H-indol-4-yloxy]-acetic acid, 3 (Compound 5-27) as follows.

The diester 2 (0.029 g, 0.026 mmole) and 1,3-dimethoxybenzene (0.02 ml, 0.152 mmole) in dry dichloromethane (3 mL), at room temperature under nitrogen, had trifluoroacetic acid (3 mL, 38.9 mmole) added. The solution was stirred for 1 h and the solvents evaporated below 25° C. The residue was triturated with ether (10 mL) and the solid removed by filtration. The solid was washed with ether (20 mL) and dried in vacuo to give the desired compound as a beige solid (0.012 g, 46%).

¹H nmr (400 MHz, DMSO-d₆) δ 7.71 (brs, 2H), 7.38 (brs, 2H), 7.11 (dd, 2H), 7.06 (dd, 2H), 6.91 (m, 2H), 6.83 (m, 2H), 6.51 (d, 2H), 4.62 (s, 4H), 4.14 (m, 4H), 3.90 (m, 4H), 2.54 (s, 6H), 1.66 (m, 8H), 1.40 (m, 4H), 1.28, 1.23 (2m, 28H).

MS (ES+) 1017.58 (M+Na), 996.51 (M+1), 995.54 (M).

### Example 11.4

### COMPOUND (5-25)

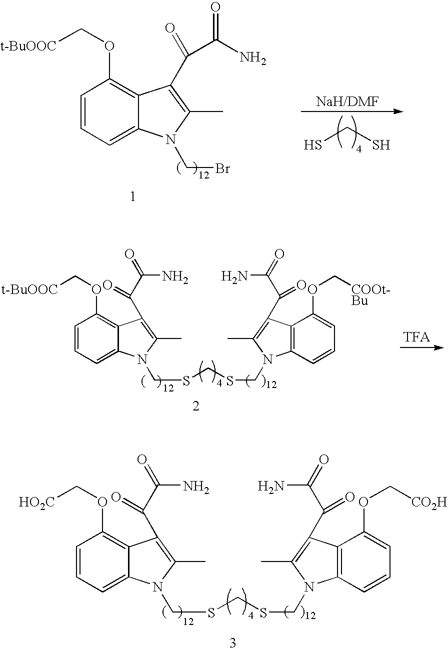

The t-Bu protected compound, tert-Butyl 2,2′-(1,1′-(12,12′-(butane-1,4-diylbis(sulfanediyl))bis(dodecane-12,1-diyl))bis(3-(2-amino-2-oxoacetyl)-2-methyl-1H-indole-4,1-diyl))bis(oxy)diacetate, 2 was first prepared as follows.

1,4-Butanedithiol (0.06 mL, 0.51 mmole) was added to 95% sodium hydride (0.028 g, 1.10 mmole) in dry DMF (4 mL), at 0° C. under nitrogen. After 0.5 h this mixture was added to the bromide 1 (0.602 g, 1.03 mmole) (prepared as in Example 11B) in dry DMF (6 mL), at 0° C. under nitrogen. The reaction was maintained at 0° C. for 9 h and slowly warmed to room temperature overnight. The mixture was cooled to 0° C., quenched with ammonium chloride solution (5 mL), diluted with dichloromethane (50 mL) and ammonium chloride solution (40 mL). The organic phase was separated and the aqueous phase extracted with dichloromethane (5×40 mL). The combined organic phase was washed with brine (50 mL), dried (Na₂SO₄), filtered and evaporated to a red/brown syrup. Purification by chromatography over silica gel, using chloroform/ethyl acetate (2:1 to 1:1) as the eluant, gave the product as an orange/brown semi-solid (0.224 g, 39%).

The resulting diester 2 in above scheme was then deprotected to form 2,2′-(1,1′-(12,12′-(Butane-1,4-diylbis(sulfanediyl))bis(dodecane-12,1-diyl))bis(3-(2-amino-2-oxoacetyl)-2-methyl-1H-indole-4,1-diyl))bis(oxy)diacetic acid, 3 (Compound 5-25)

The diester 2 (0.051 g, 0.045 mmole) and 1,3-dimethoxybenzene (0.02 ml, 0.152 mmole) in dry dichloromethane (2 mL), at room temperature under nitrogen, had trifluoroacetic acid (2 mL, 25.9 mmole) added. The solution was stirred for 1 h and the solvents evaporated below 25° C. The residue was triturated with ether (20 mL) and the solid removed by filtration. The solid was washed with ether (20 mL) and stirred with ether (7 mL) for 1 h. The product was removed by filtration and dried in vacuo to give the desired compound as a beige solid (0.029 g, 64%).

¹H nmr (400 MHz, DMSO-d₆) δ 7.71 (brs, 2H), 7.38 (brs, 2H), 7.12 (dd, 2H), 7.07 (dd, 2H), 6.52 (d, 2H), 4.62 (s, 4H), 4.15 (m, 4H), 2.54 (s, 6H), 2.45 (m, 8H), 1.66 (m, 4H), 1.57 (m, 4H), 1.48 (m, 4H), 1.29, 1.23 (2m, 32H).

MS (ES+) 1030.35 (M+Na), 1008.35 (M+1), 1007.39 (M).

### Example 11.5

### COMPOUND (5-26)

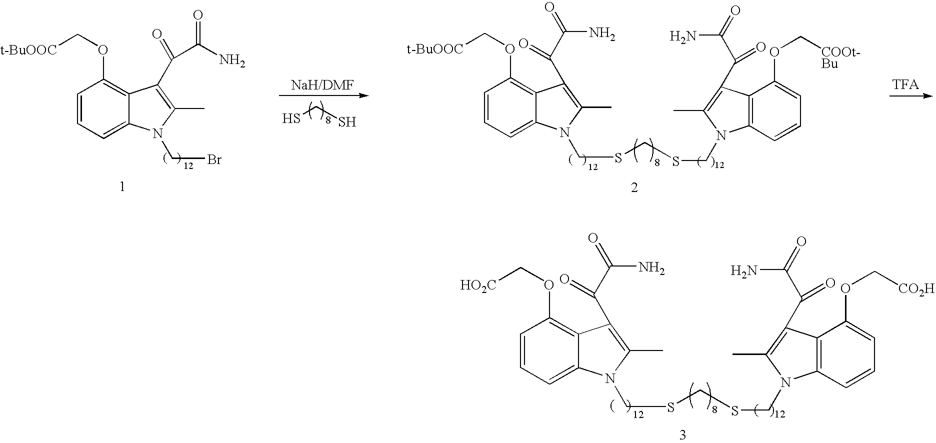

The t-Bu protected compound tert-Butyl 2,2′-(1,1′-(12,12′-(octane-1,8-diylbis(sulfanediyl))bis(dodecane-12,1-diyl))bis(3-(2-amino-2-oxoacetyl)-2-methyl-1H-indole-4,1-diyl))bis(oxy)diacetate, 2 was prepared as follows.

1,8-Octanedithiol (0.115 mL, 0.62 mmole) was added to 95% sodium hydride (0.035 g, 1.38 mmole) in dry DMF (3 mL), at 0° C. under nitrogen. After 0.5 h this mixture was added to the bromide 1 (0.760 g, 1.31 mmole) (prepared as in Example 11B) in dry DMF (9 mL), at 0° C. under nitrogen. The reaction was maintained at 0° C. for 9 h and slowly warmed to room temperature overnight. The mixture was cooled to 0° C., quenched with ammonium chloride solution (10 mL), diluted with dichloromethane (100 mL) and washed with ammonium chloride solution (2×50 mL). The organic phase was separated and the aqueous phase extracted with dichloromethane (3×30 mL). The combined organic phase was dried (Na₂SO₄), filtered and evaporated to a brown syrup. Purification by chromatography over silica gel, using chloroform/ethyl acetate (2:1 to 1:1) as the eluant, gave the product as yellow solid (0.422 g, 58%).

The resulting diester 2 in the above schema was deprotected to form 2,2′-(1,1′-(12,12′-(Octane-1,8-diylbis(sulfanediyl))bis(dodecane-12,1-diyl))bis(3-(2-amino-2-oxoacetyl)-2-methyl-1H-indole-4,1-diyl))bis(oxy)diacetic acid, 3 (Compound 5-26) as follows.

The diester 2 (0.092 g, 0.078 mmole) and 1,3-dimethoxybenzene (0.04 ml, 0.312 mmole) in dry dichloromethane (3 mL), at room temperature under nitrogen, had trifluoroacetic acid (3 mL, 38.9 mmole) added. The solution was stirred for 2 h and the solvents evaporated below 25° C. The residue was triturated with ether (30 mL) and the solid removed by filtration. The solid was washed with ether (20 mL) and stirred with ether (6 mL) for 1 h. The product was removed by filtration washed with ether (20 mL) and dried in vacuo to give the desired compound as a beige solid (0.060 g, 72%).

¹H nmr (400 MHz, DMSO-d₆) δ 7.71 (brs, 2H), 7.38 (brs, 2H), 7.12 (dd, 2H), 7.08 (dd, 2H), 6.52 (d, 2H), 4.63 (s, 4H), 4.16 (m, 4H), 2.55 (s, 6H), 2.45 (m, 8H), 1.66 (m, 4H), 1.49 (m, 8H), 130, 1.23 (2m, 40H).

MS (ES+) 1064.42 (M+1), 1063.45 (M).

### Example 11.6

### COMPOUND (5-24)

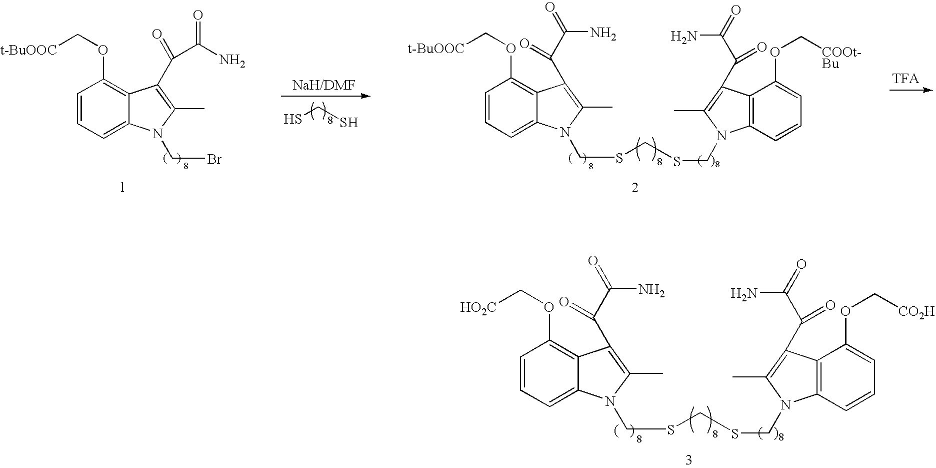

The t-Bu protected compound tert-Butyl 2,2′-(1,1′-(8,8′-(octane-1,8-diylbis(sulfanediyl))bis(octane-8,1-diyl))bis(3-(2-amino-2-oxoacetyl)-2-methyl-1H-indole-4,1-diyl))bis(oxy)diacetate, 2 was prepared as follows.

1,8-Octanedithiol (0.73 mL, 3.94 mmole) was added to sodium hydride (0.21 g, 8.75 mmole) in dry DMF (12 mL), at 0° C. under nitrogen. After 0.5 h this mixture was added to the bromide 1 (4.3 g, 8.21 mmole) (prepared as in Example 11A) in dry DMF (20 mL), at 0° C. under nitrogen. The reaction was maintained at 0° C. for 8 h and stored in the freezer overnight. The mixture was cooled to 0° C., quenched with ammonium chloride solution (15 mL), diluted with dichloromethane (100 mL) and washed with ammonium chloride solution (50 mL). The organic phase was separated and the aqueous phase extracted with dichloromethane (2×25 mL). The combined organic phase was washed with brine (75 mL) dried (Na₂SO₄), filtered and evaporated to a yellow/orange syrup. Purification by chromatography over silica gel, using chloroform/ethyl acetate (2:1 to 3:2) as the eluant, gave the product as yellow solid (2.79 g, 32%).

The resulting diester 2 in the above schema was deprotected to form 2,2′-(1,1′-(8,8′-(Octane-1,8-diylbis(sulfanediyl))bis(octane-8,1-diyl))bis(3-(2-amino-2-oxoacetyl)-2-methyl-1H-indole-4,1-diyl))bis(oxy)diacetic acid, 3 (Compound 5-24) as follows.

The diester 2 (1.97 g, 1.85 mmole) and 1,3-dimethoxybenzene (0.74 mL, 5.65 mmole) in dry dichloromethane (20 mL), at room temperature under nitrogen, had trifluoroacetic acid (20 mL, 38.9 mmole) added. The solution was stirred for 1 h and the solvents evaporated below 25° C. The residue was triturated with ether (50 mL) and the solid removed by filtration and washed with ether (100 mL). The solid was triturated with ether (50 mL), filtered and washed with ether (50 mL). The product was dried in vacuo to give the desired compound as a beige solid (1.57 g, 89%).

¹H nmr (400 MHz, DMSO-d₆) δ 7.70 (brs, 2H), 7.38 (brs, 2H), 7.13 (dd, 2H), 7.08 (dd, 2H), 6.52 (d, 2H), 4.63(s, 4H), 4.15 (m, 4H), 2.54 (s, 6H), 2.44 (m, 8H), 1.66 (m, 4H), 1.48 (m, 8H), 1.29, 1.26 (2m, 24H).

MS (ES+) 952.26 (M+1), 951.26 (M).

### Example 11.7a

### COMPOUND (5-28)

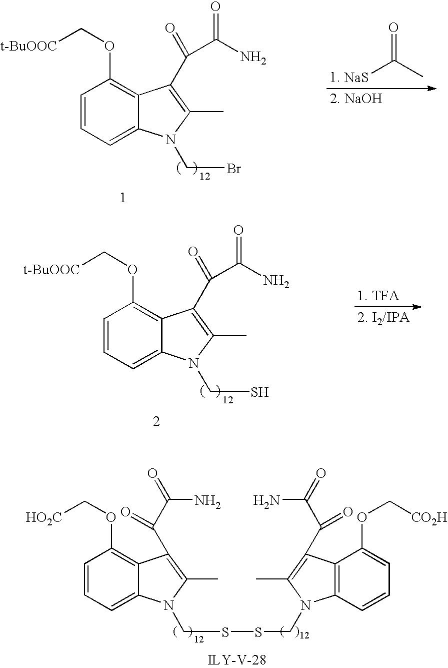

2,2′-(1,1′-(12,12′-disulfanediylbis(dodecane-12,1-diyl))bis(3-(2-amino-2-oxoacetyl)-2-methyl-1H-indole-4,1-diyl))bis(oxy)diacetic acid (ILY-V-28) To the solution of (1 mmol) of 1 in 30 mL of EtOH is added 1.1 mmol of thioacetate sodium salt. This mixture is stirred for 12 hour and then the reaction is heated to 45° C. To the resulted yellow solution 2 mL of 10% NaOH solution is added and stirred for an additional 8 hours. After the reaction is cooled to rt, solvent is removed and extracted with EtOAc. The resulted mixture is washed with water, brine, and dried over MgSO₄ to obtain a crude product. To the solution of (1 mmol) of the crude product in 15 mL of CH₂Cl₂ is added 2 mL of trifluoroacetic acid. This mixture is stirred for 1.5 hour, the solvent is evaporated at reduced pressure, and the residue is diluted with EtOAc and water. The organic phase is washed with brine, dried over MgSO₄, evaporated at reduced pressure, and purified by column chromatography to obtain the deprotected compound. To a solution of the deprotected compound (1 mmol) in isopropanol (10 mL) is added iodine (127 mg, 0.5 mmol). After 2 hours, the reaction mixture is concentrated and redissolved in EtOAc (25 mL). The solution is washed with Na₂S₂O₄ (2×10 mL) and brine (10 mL), is dried over sodium sulfate, filtered, and is concentrated in vacuo. The product is to be purified by column chromatography to provide disulfide ILY-V-28.

### Example 11.7b

### COMPOUND (5-28)

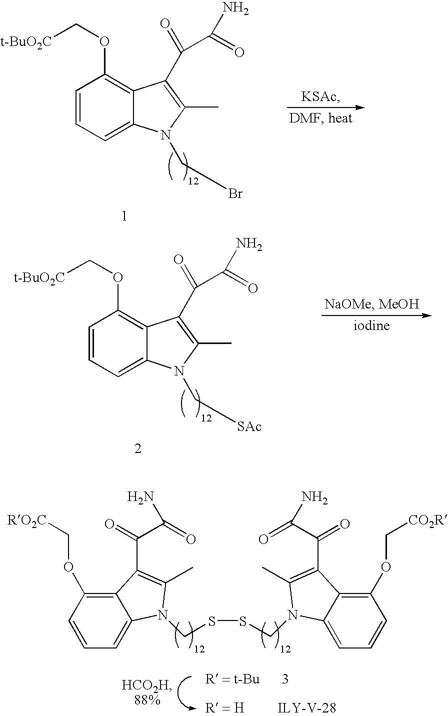

[3-Aminooxalyl-1-(12-methoxycarbonylsulfanyl-dodecyl)-2-methyl-1H-indol-4-yloxy]-acetic acid tert-butyl ester (2): A mixture of [3-aminooxalyl-1-(12-bromo-dodecyl)-2-methyl-1H-indol-4-yloxy]-acetic acid tert-butyl ester (1) (0.18 g, 0.32 mmol) and potassium acetate (0.036 g, 0.32 mmol) were heated in dry DMF (5 mL) at 70° C. for 5 h under N₂. The mixture was cooled and concentrated to dryness under high vacuum. The resulted syrup was suspended in saturated aqueous NH₄Cl solution and then extracted with EtOAc (10×3 mL). The combined organic layers were washed with water (10×2 mL) and dried (Na₂SO₄). The solvent was removed under reduced pressure, and the residue was chromatographed on a silica gel column eluting with 70% ethyl acetate in hexane to afford intermediate 2 as colorless syrup. Yield: 0.18 g, 98%.

(3-Aminooxalyl-1-{12-[1-(3-aminooxalyl-4-tert-butoxycarbonylmethoxy-2-methyl-indol-1-yl)-undecyldisulfanyl]-dodecyl}-2-methyl-1H-indol-4-yloxy)-acetic acid tert-butyl ester (3): A mixture of intermediate (2) (0.10 g, 0.17 mmol) in dry MeOH (5 mL) and a catalytic amount of iodine (0.001 g) was treated with 1N NaOMe methanol solution. The mixture was stirred at room temperature for 18 h. The solvent was removed and the residue was chromatographed on a silica gel column eluting with 70% ethyl acetate in hexane to afford intermediate 3 as an off-white solid. Yield: 0.07 g, 38%.

(3-Aminooxalyl-1-{12-[11-(3-aminooxalyl-4-carboxymethoxy-2-methyl-indol-1-yl)-undecyldisulfanyl]-dodecyl}-2-methyl-1H-indol-4-yloxy)-acetic acid (IIy-V-28): A mixture of intermediate (3) (0.06 g, 0.056 mmol) in aqueous HCO₂H (88%, 2 mL) was stirred at room temperature for 6 h. The mixture was concentrated to dryness under high vacuum and co-evaporated with water (2×2 mL). The flask containing the gummy material was then transferred to freeze dryer and was kept under high vacuum overnight to get the title compound IIy-V-28 as a pale green solid. Yield: 0.05 g, 92%. ¹H NMR: (DMSO-d₆), δ, ppm: (5-37-159) δ 7.72 (bs, 1H), 7.41 (bs, 1H), 7.17 (t, 1H), 7.10 (t, 1H), 6.46 (d, 1H), 4.62 (s, 2H), 4.08 (t, 3H), 2.62 (t, 2H), 2.45 (s, 3H), 1.70-1.60 (m, 2H), 1.48-1.41 (m, 2H), 1.38-1.15 (m, 40H). ES-MS: m/z=951.3 (M+1)

### Example 11.8a

### COMPOUND (5-29)

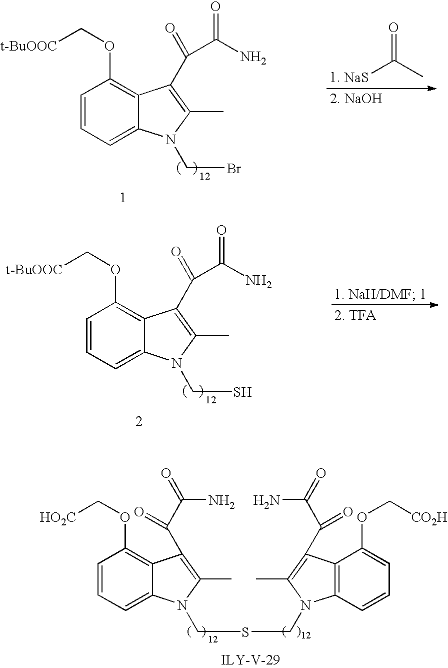

2,2′-(1,1′-(12,12′-thiobis(dodecane-12,1-diyl))bis(3-(2-amino-2-oxoacetyl)-2-methyl-1H-indole-4,1-diyl))bis(oxy)diacetic acid (ILY-V-29) To the solution of (1 mmol) of 1 in 30 mL of EtOH is added 1.1 mmol of thioacetate sodium salt. This mixture is stirred for 12 hour and then the reaction is heated to 45° C. To the resulted yellow solution 2 mL of 10% NaOH solution is added and stirred for an additional 8 hours. After the reaction is cooled to rt, solvent is removed and extracted with EtOAc. The resulting mixture is to be washed with water, brine, and dried over MgSO₄ to obtain a crude product of. The material then is purified by column chromatography to give 2.

Compound 2 (0.9 mmole) is added to sodium hydride (1.2 mmole) in dry DMF (12 mL), at 0° C. under nitrogen. After 0.5 h this mixture is added to the bromide 1 (0.95 mmole) in dry DMF (20 mL), at 0° C. under nitrogen. The reaction is maintained at 0° C. for 8 h and quenched with ammonium chloride solution (15 mL), diluted with dichloromethane (100 mL) and washed with ammonium chloride solution (50 mL). The organic phase is separated and the aqueous phase extracted with dichloromethane (2×25 mL). The combined organic phase is washed with brine (75 mL) dried (Na₂SO₄), filtered and evaporated to a yellow/orange syrup. Purification by chromatography over silica gel, using chloroform/ethyl acetate as the eluant, can give the protected dimer product.

The dimer product (0.9 mmole) and 1,3-dimethoxybenzene (3 mmole) in dry dichloromethane (20 mL), at room temperature under nitrogen, is added with trifluoroacetic acid (10 mL). The solution is stirred for 1 h and the solvents evaporated below 25° C. The residue is triturated with ether (50 mL) and the solid is removed by filtration and is washed with ether (100 mL). The solid is triturated with ether (50 mL), filtered and washed with ether (50 mL). The product is dried in vacuo to give ILY-V-29.

### Example 11.8b

### Compound (5-29)

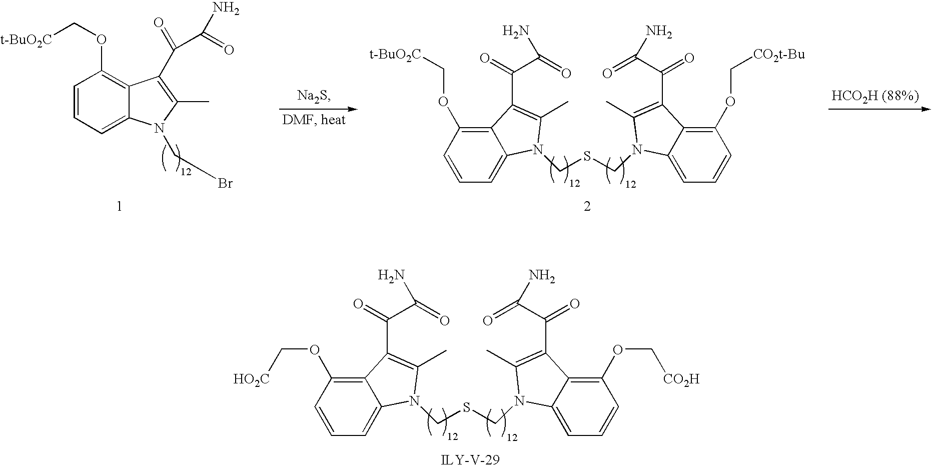

(3-Aminooxalyl-1-{12-[12-(3-aminooxalyl-4-tert-butoxycarbonylmethoxy-2-methyl-indoyl-1-yl)-dodecylsulfanyl]-dodecyl}-2-methyl-1H-4-yloxy-acetic acid tert-butyl ester (2): A mixture of [3-aminooxalyl-1-(12-bromo-dodecyl)-2-methyl-1H-indol-4-yloxy]-acetic acid tert-butyl ester (1) (0.285 g, 0.38 mmol) and sodium sulfide (0.01 g, 0.12 mmol) were heated in dry DMF (5 mL) at 70° C. for 5 h under N₂. The reaction mixture was cooled and concentrated. The resulted syrup was suspended in saturated aqueous NH₄Cl solution, extracted with CH₂Cl₂ (10×3 mL) and the combined organic layers were washed with water (5×2 mL) and dried over Na₂SO₄. The solvent was removed under reduced pressure, and the residue was chromatographed on a silica gel column, eluting with 70% ethyl acetate in hexanes to afford intermediate 5 as an off-white solid. Yield: 0.13 g, 96%.

(3-Aminooxalyl-1{-12-[12-(3-aminooxalyl-4-carboxymethoxy-2-methyl-indol-1-yl)-dodecylsulfanyl]-dodecyl}-2-methyl-1H-indol-4-yloxy)-acetic acid (IIy-V-29): A solution of intermediate (2) (0.04 g, 0.038 mmol) in aqueous HCO₂H (88%, 2 mL) was stirred at room temperature for 6 h. The mixture was concentrated to dryness under high vacuum and co-evaporated with water (2×2 mL). The flask containing the gummy material was then transferred to freeze dryer and was kept under high vacuum overnight to get the title compound IIy-V-29 as a pale yellow powder. Yield: 0.03 g, 90% ¹H NMR: (DMSO-d₆), 6, ppm: (5-37-145) δ 7.70 (bs, 1H), 7.40 (bs, 1H), 7.15 (t, 1H), 7.10 (t, 1H), 6.46 (d, 1H), 4.62 (s, 2H), 4.18 (t, 3H), 2.45 (s, 3H), 2.20 (t, 2H), 1.70-1.60 (m, 2H), 1.48-1.41 (m, 2H), 1.39-1.15 (m, 40H). ES-MS: m/z=920.3 (M+1)

### Example 11.9a

### COMPOUND 5-30

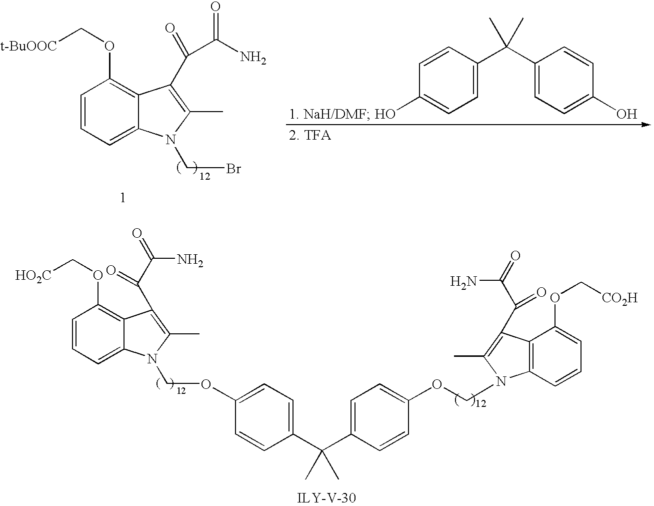

2,2′-(1,1′-(12,12′-(4,4′-(propane-2,2-diyl)bis(4,1-phenylene))bis(oxy)bis(dodecane-12,1-diyl))bis(3-(2-amino-2-oxoacetyl)-2-methyl-1H-indole-4,1-diyl))bis(oxy)diacetic acid (ILY-V-30) Bisphenol A (1 mmole) is added to sodium hydride (2.2 mmole) in dry DMF (12 mL), at 0° C. under nitrogen. After 0.5 h this mixture is added to the bromide 1 (2.05 mmole) in dry DMF (20 mL), at 0° C. under nitrogen. The reaction is maintained at 0° C. for 8 h and quenched with ammonium chloride solution (15 mL), diluted with dichloromethane (100 mL) and is washed with ammonium chloride solution (50 mL). The organic phase is separated and the aqueous phase extracted with dichloromethane (2×25 mL). The combined organic phase is washed with brine (75 mL) dried (Na₂SO₄), filtered and evaporated to a yellow/orange syrup. Purification by chromatography over silica gel, using chloroform/ethyl acetate as the eluant, give the protected dimer product.

The dimer product (0.9 mmole) and 1,3-dimethoxybenzene (3 mmole) in dry dichloromethane (20 mL), at room temperature under nitrogen, is added with trifluoroacetic acid (10 mL). The solution is stirred for 1 h and the solvents evaporated below 25° C. The residue is triturated with ether (50 mL) and the solid is removed by filtration and is washed with ether (100 mL). The solid is triturated with ether (50 mL), filtered and washed with ether (50 mL). The product is to be dried in vacuo to give ILY-V-30.

### Example 11.9b

### Compound 5-30

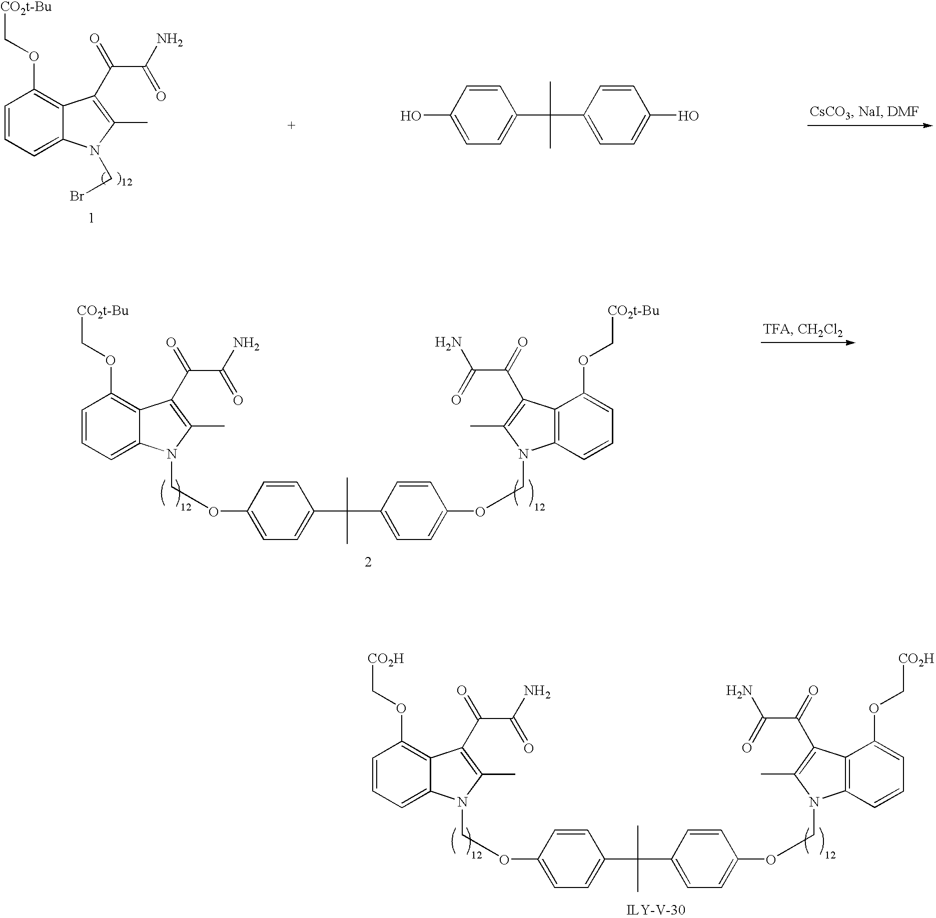

(3-Aminooxalyl-1-{12-[4-(1-{4-[12-(3-aminooxalyl-4-tert-butoxycarbonylmethoxy-2-methyl-indol-1-yl)-dodecyloxy]-phenyl}-1-methyl-ethyl)-phenoxy]-dodecyl}-2-methyl-1H-indol-4-yloxy)-acetic acid tert-butyl ester (2): To a solution of bisphenol (10.27 g, 0.045 mole) in anhydrous DMF (700 mL), cesium carbonate (147 g, 0.45 mole) was added. The mixture was stirred at room temperature for 30 minutes. To the mixture [3-aminooxalyl-1-(12-bromo-dodecyl)-2-methyl-1H-indol-4-yloxy]-acetic acid tert-butyl ester (1) (57.8 g, 0.10 mole) and sodium iodide (33.5 g, 0.225 mole) were added. The reaction mixture was stirred at room temperature for 18 h. The mixture was diluted with ethyl acetate (3.5 L) and washed with water (4×700 mL) and brine (1×700 mL). The organic layer was separated and dried with sodium sulphate, then concentrated. The residue was purified by column chromatography (2:1 EtOAc:CHCl₃) to afford intermediate (2) as a white solid. Yield: 48 g, 87%

(3-Aminooxalyl-1-{12-[4-(1-{4-[12-(3-aminooxalyl-4-carboxymethoxy-2-methyl-indol-1-yl)-dodecyloxy]-phenyl}-1-methyl-ethyl)-phenoxy]-dodecyl}-2-methyl-1H-indol-4-yloxy)-acetic acid (IIy-V-30): To a solution of intermediate (2) (23 g, 0.0187 mole) in dichloromethane (1 L), trifluoroacetic acid (230 mL, 1.131 mole) was added dropwise. The reaction mixture was stirred at room temperature for 3 h. The reaction solvent was evaporated and the brown sticky residue was stirred in diethyl ether (700 mL) for 2 h. The resulting solid was collected by filtration and dried under high vacuum for 18 h to afford IIy-V-30 as a pink solid. Yield: 22.1 g>100% (contains some inorganic salts). ¹H NMR (400 MHz, DMSO-d₆) δ, ppm: 12.86 (brs, 2H), 7.72 (s, 2H), 7.40 (s, 2H), 7.18-7.04 (m, 8H), 6.78 (d, 4H), 6.50 (d, 2H), 4.42 (s, 4H), 4.17 (brt, 4H), 3.87 (t, 4H), 2.50 (s, 6H), 1.78-1.20 (m, 22H). ES-MS: m/z=1113.28 (M+1)

### Example 11.10.1a

### COMPOUND (5-31)

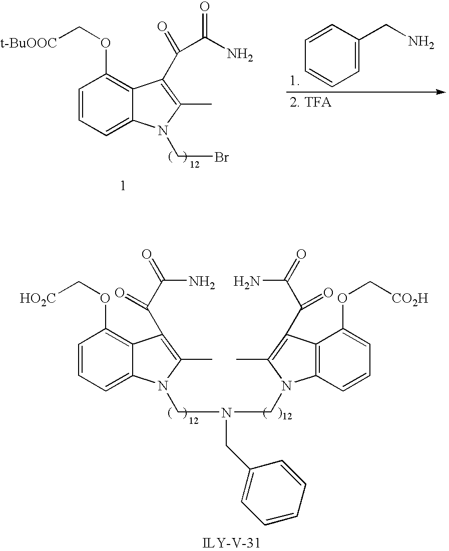

2,2′-(1,1′-(12,12′-(benzylazanediyl)bis(dodecane-12,1-diyl))bis(3-(2-amino-2-oxoacetyl)-2-methyl-1H-indole-4,1-diyl))bis(oxy)diacetic acid (ILY-V-31) Benzyl amine (1 mmole) is added to the bromide 1 (2.05 mmole) in dry DMF (12 mL) at rt under nitrogen. The reaction is maintained at 50° C. for 8 h and is quenched with ammonium chloride solution (15 mL), is diluted with dichloromethane (100 mL) and is washed with ammonium chloride solution (50 mL). The organic phase is separated and the aqueous phase is extracted with dichloromethane (2×25 mL). The combined organic phase is washed with brine (75 mL) dried (Na₂SO₄), is filtered and is evaporated to a yellow/orange syrup. Purification by chromatography over silica gel, using chloroform/ethyl acetate as the eluant, can give the protected dimer product.

The protected dimer product (0.9 mmole) and 1,3-dimethoxybenzene (3 mmole) in dry dichloromethane (20 mL), at room temperature under nitrogen, is added with trifluoroacetic acid (10 mL). The solution is stirred for 1 h and the solvents evaporated below 25° C. The residue is triturated with ether (50 mL) and the solid removed by filtration and washed with ether (100 mL). The solid is triturated with ether (50 mL), filtered and washed with ether (50 mL). The product is dried in vacuo to give ILY-V-31.

### Example 11.10.1b and 11.10.2

### COMPOUND (5-31) AND (5-45)

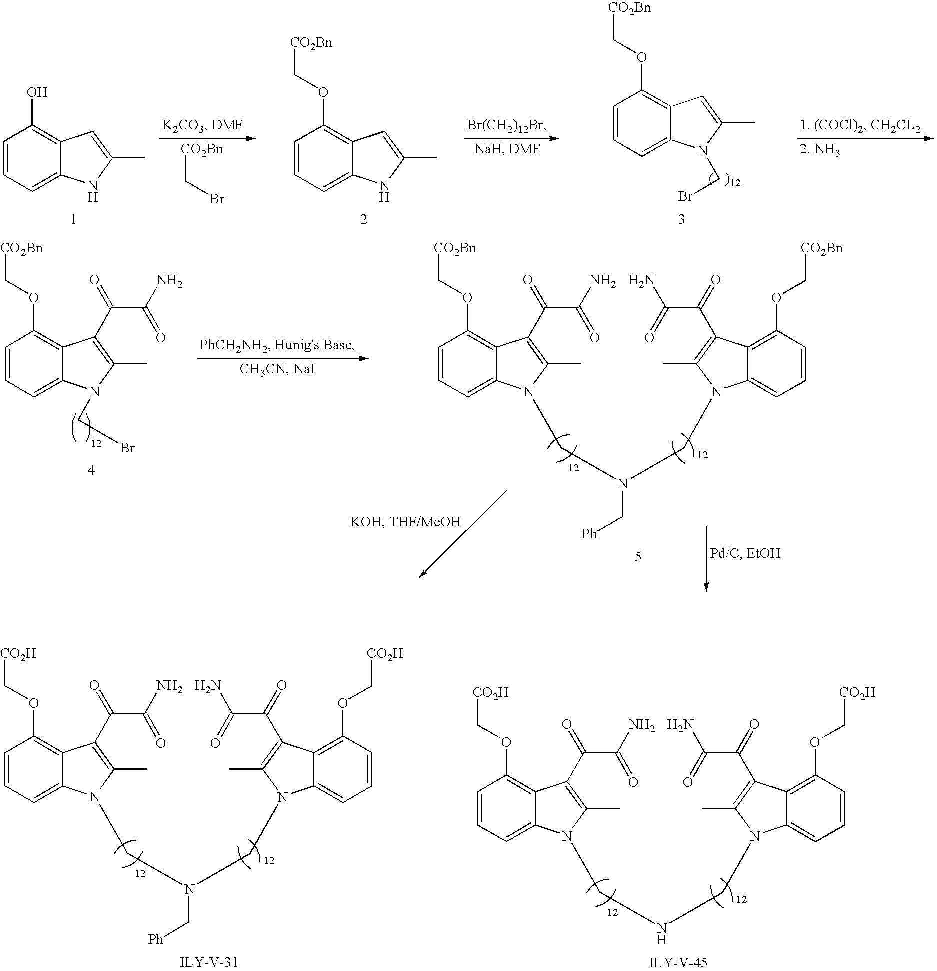

(2-Methyl-1H-indol-4-yloxy)-acetic acid benzyl ester (2): A mixture of 4-hydroxy-2-methylindole (3.0 g, 0.02 mole), bromo-acetic acid benzyl ester (4.6 g, 0.02 mole), potassium carbonate (2.8 g, 0.02 mole) in acetone was refluxed for 48 h. The reaction mixture was filtered and the filtrate was concentrated. The residue was purified by column chromatography (10:1 Hex:EtOAc) to afford intermediate (2). Yield: 3.5 g, 58%.

[1-(12-Bromododecyl)-2-methyl-1H-indol-4-yloxy]-acetic acid benzyl ester (3): To a suspension of sodium hydride (60% in mineral oil, 0.093 g, 6.45 mmole) in DMF (10 mL), (2-methyl-1H-indol-4-yloxy)-acetic acid benzyl ester (2) (0.845 g, 2.3 mmole) was added. The mixture was stirred at room temperature for 1 h. To the mixture, dibromododecane (0.765 g, 2.3 mmole) was added and the reaction mixture was stirred at room temperature for 18 h. The reaction was diluted with ethyl acetate and washed with water. The organic layer was separated, dried with sodium sulphate and concentrated. The residue was purified by column chromatography (10:1, hexane:EtOAc) to afford intermediate 3. Yield: 0.708 g, 45%.

[3-Aminooxalyl-1-(12-bromododecyl)-2-methyl-1H-indol-4-yloxy]-acetic acid benzyl ester (4): To a solution of intermediate 3 (0.708 g, 1.31 mmole) in anhydrous dichloromethane (20 mL), oxalyl chloride (0.166 g, 1.31 mmole) was added dropwise. The mixture was stirred for 2 h, and then ammonia was bubbled through the mixture for 15 min. The reaction mixture was evaporated to afford intermediate 4 (1.0 g) as a crude mixture which was used in the subsequent reaction without further purification.

[3-Aminooxalyl-1-(12-{[12-(3-aminooxalyl-4-benzyloxycarbonylmethoxy-2-methyl-indol-1-yl)-dodecyl]-benzylamino}-dodecyl)-2-methyl-1H-indol-4-yloxy]-acetic acid benzyl ester (5): A mixture of intermediate 4 (1.0 g, crude martial from the previous step), benzylamine (0.08 g, 0.74 mmole), sodium iodide (0.005 g) and Hunig's base (0.084 g, 0.65 mmole) in acetonitrile (10 mL) was refluxed for 12 h. The mixture was concentrated and the residue was purified by column chromatography (100% CH₃CN) to afford intermediate 5 as a solid. Yield 0.41 g, 26% for 2 steps.

(3-Aminooxalyl-1-{12-[12-(3-aminooxalyl-4-carboxymethoxy-2-methyl-indol-1-yl)-dodecylamino]-dodecyl}-2-methyl-1H-indol-4-yloxy)-acetic acid (IIy-V-45): To a solution of intermediate 5 (0.098 g, 0.084 mmole) in ethanol (10 mL), Pd/C (10%, 50 mg) was added. The mixture was stirred under hydrogen atmosphere using a balloon for 30 minutes. The mixture was filtered through Celite and the filtrate was evaporated. The resulting solid was washed with chloroform and hexane to afford IIy-V-45. Yield: 0.022 g, 27%. ¹H NMR (400 MHz, DMSO-d₆) δ, ppm: 8.40 (brs, 1H), 7.78 (brs, 2H), 7.41 (brs, 2H), 7.01-7.12 (m, 4H), 6.45 (d, 2H), 4.61 (s, 4H), 4.18(t, 4H), 2.80(t, 4H), 2.54(s, 6H), 1.62(m, 4H), 1.45(m, 4H), 1.11-1.18(m, 32H) ppm. ES-MS: m/z=902.15 (M+1).

[3-Aminooxalyl-1-(12-{[12-(3-aminooxalyl-4-carboxymethoxy-2-methyl-indol-1-yl)-dodecyl]-benzylamino}-dodecyl)-2-methyl-1H-indol-4-yloxy]-acetic acid (IIy-V-31): To a solution of intermediate 5 (0.204 g, 0.204 mmole) in THF/MeOH (5 mL/5 mL), a solution of potassium hydroxide (0.20 g, 3.57 mmole) in water (1 mL) was added. The reaction mixture was stirred at room temperature for 18 h. The reaction was acidified to pH 5 with 2 M HCl. The solvent was evaporated and the residue was washed with diethyl ether (2×10 mL). The solid was collected by filtration and dried to afford IIy-V-31 as a yellow solid. Yield: 0.190 g, 93%. ¹H NMR (400 MHz, DMSO-d₆) δ, ppm: 7.78 (brs, 2H), 7.40-7.00 (m, 11H), 6.50 (d, 2H), 4.60 (s, 4H), 4.18 (brs, 4H), 3.63 (brs, 2H), 3.42-3.20 (m, 4H), 2.55 (s, 6H), 1.65 (brs, 4H), 1.42 (brs, 4H), 1.38-1.08 (m, 14H). ES-MS: m/z=993.38 (M+1).

### Example 11.11a

### COMPOUND (5-32)

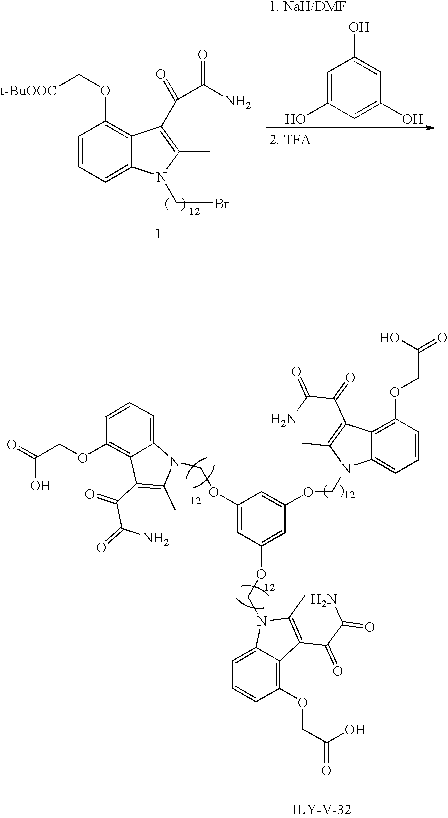

2,2,′,2″-(1,1′,1″-(12,12′,12″-(benzene-1,3,5-triyltris(oxy))tris(dodecane-12,1-diyl))tris(3-(2-amino-2-oxoacetyl)-2-methyl-1H-indole-4,1-diyl))tris(oxy)triacetic acid (ILY-V-32) Dehydrated phloroglucinol (1 mmole) is added to sodium hydride (3.3 mmole) in dry DMF (12 mL), at 0° C. under nitrogen. After 0.5 h this mixture is added to the bromide 1 (3.1 mmole) in dry DMF (20 mL), at 0° C. under nitrogen. The reaction is maintained at 0° C. for 8 h and is quenched with ammonium chloride solution (15 mL), is diluted with dichloromethane (100 mL) and is washed with ammonium chloride solution (50 mL). The organic phase is separated and the aqueous phase extracted with dichloromethane (2×25 mL). The combined organic phase is washed with brine (75 mL) dried (Na₂SO₄), filtered and evaporated to a yellow/orange syrup. Purification by chromatography over silica gel, using chloroform/ethyl acetate as the eluant, can give the protected dimer product.

The protected dimer product (0.9 mmole) and 1,3-dimethoxybenzene (3 mmole) in dry dichloromethane (20 mL), at room temperature under nitrogen, is added with trifluoroacetic acid (10 mL). The solution is stirred for 1 h and the solvents evaporated below 25° C. The residue is triturated with ether (50 mL) and the solid removed by filtration and washed with ether (100 mL). The solid is triturated with ether (50 mL), is filtered and is washed with ether (50 mL). The product is dried in vacuo to give ILY-V-32.

### Example 11.11b

### COMPOUND (5-32)

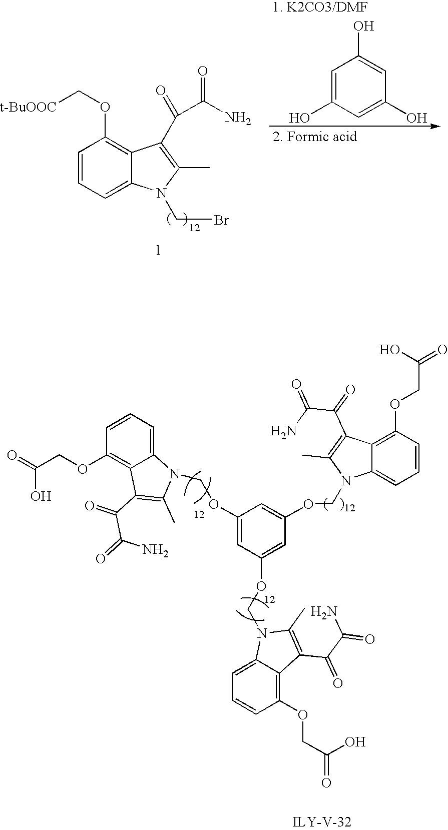

[3-Aminooxalyl-1-(12-{3,5-bis-[12-(3-aminooxalyl-4-tert-butoxycarbonylmethoxy-2-methyl-indol-1-yl)-dodecyloxy]-phenoxy}-dodecyl)-2-methyl-1H-indol-4-yloxy]-acetic acid tert-butyl ester: A mixture of [3-aminooxalyl-1-(12-bromododecyl)-2-methyl-1H-indol-4-yloxy]-acetic acid tert-butyl ester (1) (0.70 g, 1.11 mmol), K₂CO₃ (1.0 g, excess) and phloroglucinol (0.03 g, 0.18 mmol) were heated in dry DMF (8 mL) at 55° C. for 12 h under N₂. The mixture was cooled and concentrated to dryness. The syrup was suspended in CH₂Cl₂ (50 mL) and filtered through Celite. The filtrate was washed with water (10×2 mL) and dried (Na₂SO₄). The solvent was removed under reduced pressure, and the residue was chromatographed on a silica gel column eluting with 70% ethyl acetate in hexane to afford the intermediate as an off-white solid. Yield: 0.09 g, 30%.

[3-Aminooxalyl-1-(12-{3,5-bis-[12-(3-aminooxalyl-4-carboxymethoxy-2-methyl-indol-1-yl)-dodecyloxy]-phenoxy}-dodecyl)-2-methyl-1H-indol-4-yloxy]-acetic acid (IIy-V-32): The intermediate (0.08 g, 0.049 mmol) was dissolved in aqueous HCO₂H (88%, 2 mL) and the mixture was stirred at room temperature for 6 h. The mixture was concentrated to dryness under high vacuum and co-evaporated with water (2×2 mL). The flask containing the gummy material was then transferred to freeze dryer and was under high vacuum overnight to get the title compound IIy-V-32 as a pale green gum. Yield: 0.03 g, 40%. ¹H NMR: (DMSO-d₆), δ, ppm: (5-37-147) δ 7.71 (bs, 3H), 7.40 (bs, 3H), 7.20-7.05 (m, 6H), 6.48 (d, 3H), 6.01 (s, 3H), 4.62 (s, 6H), 4.18 (t, 6H), 3.83 (t, 6H), 2.47 (s, 9H), 1.70-1.60 (m, 6H), 1.38-1.05 (m, 60H). ES-MS: m/z=1452.8 (M+1).

### Example 11.12a

### COMPOUND (5-33)

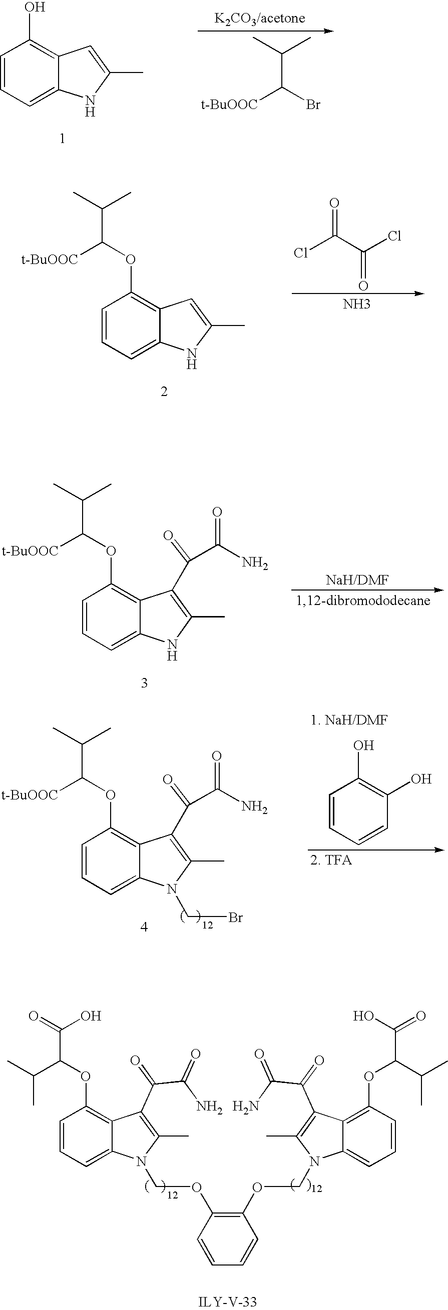

2,2′-(1,1′-(12,12′-(1,2-phenylenebis(oxy))bis(dodecane-12,1-diyl))bis(3-(2-amino-2-oxoacetyl)-2-methyl-1H-indole-4,1-diyl))bis(oxy)bis(3-methylbutanoic acid) (ILY-V-33) Hydroxy indole 1 (1 mmol) and tert-butyl 2-bromo-3-methylbutanoate (1 mmol) is dissolved in 10 mL acetone. To this solution at room temperature is added anhydrous potassium carbonate (2 mmol) and the stirred mixture is refluxed for 12 hours. The solid is removed by filtration and followed by column chromatography to give 2.

Compound 2 (1 mmole) is dissolved in anhydrous dichloromethane (50 mL). To the solution oxalyl chloride (1.1 mmole) is added. The mixture is left to stir at room temperature for 2 h. NH₃ gas is then bubbled through the solution for 30 minutes. The mixture is left to stir at room temperature for 1 h. The dichloromethane is evaporated and the residue is dissolved in ethyl acetate (200 mL) and washed with H₂O (3×200 mL) and brine (1×300 mL). The organic layer is separated, dried with magnesium sulfate and concentrated to afford 3.

The indole intermediate 3 (1 mmole) in dry DMF (10 mL), at 0° C. under nitrogen, is added with 95% sodium hydride (1.2 mmole). The mixture is stirred at 0° C. for 0.5 h and then added dropwise over 10 minutes to a solution of 1,12-dibromododecane (1.5 mmole) in dry DMF (20 mL) at 0° C. The mixture is stirred at 0° C. for 5 h and at room temperature for 19 h. The reaction 1s cooled to 0° C., quenched with ammonium chloride solution (10 mL), and diluted with dichloromethane (100 mL). The mixture is washed with ammonium chloride solution (50 mL) and the aqueous phase extracted with dichloromethane (4×25 mL). The combined organic phase is washed with brine (100 mL), dried (Na₂SO₄), filtered and evaporated to a red/brown liquid which is further evaporated under high vacuum. The residue is purified by chromatography over silica gel to give 4.

Catechol (1 mmole) is added to sodium hydride (2.2 mmole) in dry DMF (12 mL), at 0° C. under nitrogen. After 0.5 h this mixture is added to the bromide 4 (2.05 mmole) in dry DMF (20 mL), at 0° C. under nitrogen. The reaction is maintained at 0° C. for 8 h and quenched with ammonium chloride solution (15 mL), diluted with dichloromethane (100 mL) and washed with ammonium chloride solution (50 mL). The organic phase is separated and the aqueous phase extracted with dichloromethane (2×25 mL). The combined organic phase is washed with brine (75 mL) dried (Na₂SO₄), filtered and evaporated to a yellow/orange syrup. Purification by chromatography over silica gel, using chloroform/ethyl acetate as the eluant, give the protected dimer product.

The protected dimer product (0.9 mmole) and 1,3-dimethoxybenzene (3 mmole) in dry dichloromethane (20 mL), at room temperature under nitrogen, is added with trifluoroacetic acid (10 mL). The solution is stirred for 1 h and the solvents evaporated below 25° C. The residue is triturated with ether (50 mL) and the solid removed by filtration and washed with ether (100 mL). The solid is triturated with ether (50 mL), filtered and washed with ether (50 mL). The product is dried in vacuo to give ILY-V-33.

### Example 11.12b

### Compound (5-33)

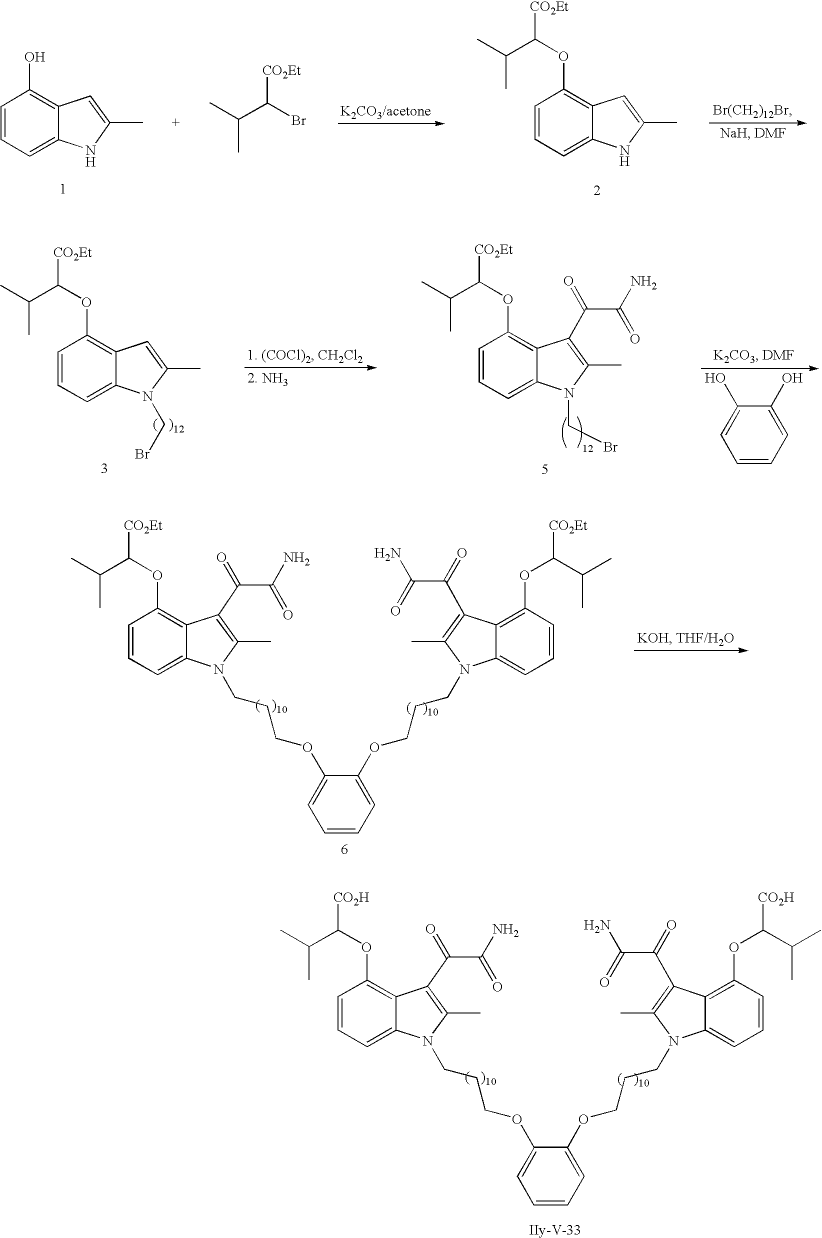

3-Methyl-2-(2-methyl-1H-indol-4-yloxy)-butyric acid ethyl ester (2): A mixture of 4-hydroxy-2-methylindole (1) (1.5 g, 0.010 mole), 2-bromo-3-methyl-butyric acid ethyl ester (2.2 g, 0.010 mole) and potassium carbonate (excess) in acetone (50 mL) was refluxed for 3 days. The reaction mixture was filtered, and the filtrate was concentrated. The residue was purified by column chromatography (20:1 Hex:EtOAc) to afford intermediate 2. Yield: 1.88 g, 71%

2-[1-(12-Bromo-dodecyl)-2-methyl-1H-indol-4-yloxy]-3-methylbutyric acid ethyl ester (3): To a mixture of NaH (60% in mineral oil, 0.42 g, 10 mmole) in anhydrous DMF (20 mL), 3-methyl-2-(2-methyl-1H-indol-4-yloxy)-butyric acid ethyl ester (2) (1.88 g, 7.0 mmole) and dibromododecane (2.30 g, 7.0 mmole) were added. The mixture was stirred at room temperature for 18 h. The reaction was diluted with ethyl acetate (50 mL) and washed with water (3×30 mL). The organic layer was separated, dried over sodium sulphate and concentrated. The residue was purified by column chromatography (10:1 Hex:EtOAc) to afford intermediate (3) Yield: intermediate (3) 1.32 g, 35%, by-product (4) 1.56 g, 31%.

2-[3-Aminooxalyl-1-(12-bromo-dodecyl)-2-methyl-1H-indol-4-yloxy]-3-methyl-butyric acid ethyl ester (5): To a solution of intermediate 3 (0.50 g, 0.959 mmole) in anhydrous dichloromethane (200 mL), oxalyl chloride (0.12 g, 0.95 mmole) was added at 0° C. The mixture was stirred for 1 h. Ammonia gas was bubbled through the reaction mixture for 20 minutes. The mixture was stirred for an addition hour and then concentrated. The residue was diluted with ethyl acetate (30 mL) and washed with water (3×30 mL). The organic layer was separated, dried over sodium sulphate and concentrated to afford intermediate (5) as a yellow solid. Yield: 0.44 g, 77%

2-{3-Aminooxalyl-1-[12-(2-{12-[3-aminooxalyl-4-(1-ethoxycarbonyl-2-methyl-propoxy)-2-methyl-indol-1-yl]-dodecyloxy}-phenoxy)-dodecyl]-2-methyl-1H-indol-4-yloxy}-3-methyl-butyric acid ethyl ester (6): A mixture of intermediate 5 (474 mg, 0.8 mmol), catechol (40 mg, 0.36 mmol) and potassium carbonate (excess) in DMF (5 mL) was stirred at room temperature for 72 h. The reaction was filtered and the filtrate was poured onto crushed ice (20 mL). The mixture was extracted with dichloromethane (3×30 mL). The organic layer was separated, dried over sodium sulphate and concentrated. The residue was purified by column chromatography (1% MeOH in CHCl₃) to afford intermediate (6) and recovered intermediate (5) (205 mg). Yield: 0.060 g, 7%.

2-(3-Aminooxalyl-1-[12-(2-{12-[3-aminooxalyl-4-(1-carboxy-2-methyl-propoxy)-2-methyl-indol-1-yl]-dodecyloxy)-phenoxy)-dodecyl]-2-methyl-1H-indol-4-yloxy}-3-methyl-butyric acid (IIy-V-33): To a solution of intermediate 6 (55 mg, 0.05 mmol) in THF/CH₃OH/H₂O (1:1:1, 2 mL:2 mL:2 mL), potassium hydroxide (0.06 g, 0.11 mmole) was added. The mixture was stirred at room temperature for 4 h. The solution was evaporated and the residue was neutralized with 1M HCl at 0° C. The solid was collected by filtration and washed with water and then hexane to afford IIy-V-33 as a yellow solid. Yield: 0.035 g, 67%. ¹H NMR (400 MHz, DMSO-d₆), δ, ppm: δ 12.51(brs, 2H), 8.10(brs, 2H), 7.62 (brs, 2H), 7.11-7.14(m, 4H), 7.92-7.96 (m, 2H), 7.81-7.84 (m, 2H), 6.42(d, 2H), 4.68(d, 2H), 4.15 (t, 4H), 3.92 (t, 4H), 2.44 (s, 6H), 2.23(m, 2H), 1.62(m, 4H), 1.20-1.43(m, 36H), 1.08(d, 6H), 0.98(d, 6H) ppm. ES-MS: m/z=1079.44(M+1).

### Example 11.13

### COMPOUND (5-23)

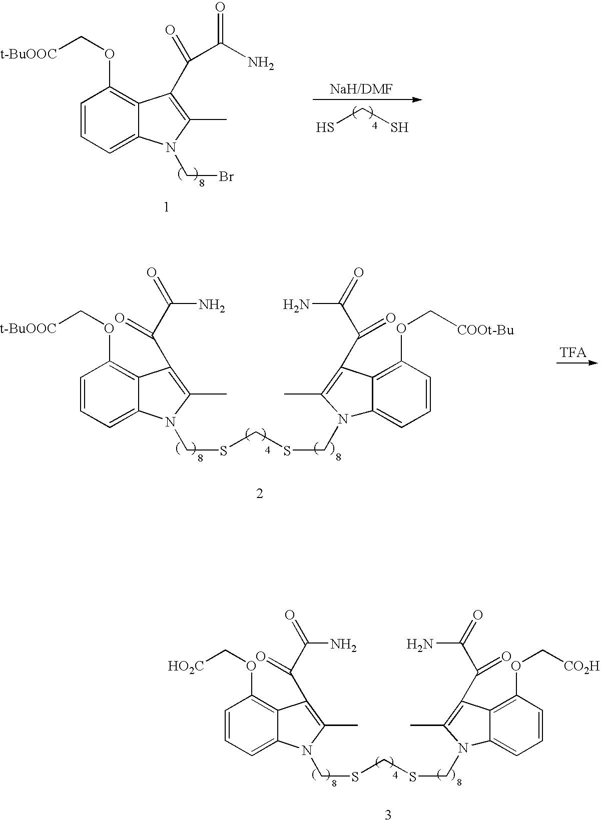

2,2′-(1,1′-(8,8′-(butane-1,4-diylbis(sulfanediyl))bis(octane-8,1-diyl))bis(3-(2-amino-2-oxoacetyl)-2-methyl-1H-indole-4,1-diyl))bis(oxy)diacetic acid (ILY-V-23) A solution of 1,4-butanedithiol (280 μL, 2.4 mmol, 290 mg) in 5 mL of anhydrous DMF was cooled in an ice bath and dry sodium hydride (125 mg, 5.23 mmol, 2.2 equiv) was added. After stirring under nitrogen for 15 min at 0° C., the mixture was transferred drop wise into a solution of N1-bromoalkyl indole 1 (2.6 g, 5.0 mmol, 2.1 equiv) in 10 mL of anhydrous DMF also cooled in an ice bath. The resulting orange mixture was stirred under nitrogen for 8 h at 0° C. After an overnight refrigerating at −20° C., the reaction mixture was quenched with 10 mL of NH₄Cl, and it was then allowed to warm to RT. It was diluted with 50 mL of DCM, washed with NH₄Cl (25 mL) and twice with brine (2×30 mL), dried over MgSO₄ and concentrated in vacuo to afford the crude product as an orange oil. Purification by flash-chromatography (H/EA: 2/3, 3/7 and 1/4) yielded the pure dimer (1.2 g, 51%) as a yellow solid.

¹H NMR (CD₂Cl₂, 300 MHz): δ 7.14 (dd, 2H, J=8.1, 8.1 Hz, H-6), 7.08 (d, 2H, J=8.1, H-5), 6.6 (br s, 2H, NH₂), 6.51 (d, 2H, J=8.1 Hz, H-7), 6.0 (br s, 2H, NH₂), 4.59 (s, 4H, H-10), 4.09 (t, 4H, J=7.8 Hz, H-14), 2.59 (s, 6H, H-9), 2.50 (m, 8H, S—CH₂), 1.78 (m, 4H, CH₂), 1.66 (m, 4H, CH₂), 1.57 (m, 4H, CH₂), 1.47 (s, 18H, C(CH₃)₃), 1.36 (m, 16H, CH₂).

¹³C NMR (CD₂Cl₂, 75.5 MHz): δ 188.5 (12), 168.3 (11), 167.5 (13), 152.0 (4), 144.2 (1), 137.8 (8), 123.2 (3), 117.0 (6), 110.3 (5), 104.1 (7+2), 82.1 (C(CH₃)₃), 66.3 (10), 44.0 (14), 36.4 (CH₂), 32.1 (CH₂), 31.7 (CH₂), 31.2 (CH₂), 29.8 (CH₂), 29.4 (CH₂), 29.3 (CH₂), 29.0 (CH₂), 28.0 (C(CH₃)₃), 27.1 (CH₂), 11.6 (9).

MS (ESI, MeOH): m/z 1029.5 [M+Na]⁺.

The protected dimer 2 (1.0 g, 1 mmol) was stirred under nitrogen with TFA (7.5 mL, 11 g, 100 mmol, 100 equiv) for 45 nm at RT. TFA in excess was then evaporated under reduced pressure to afford the crude product as a brown-yellow oil. Purification by reversed-phase chromatography (Water/Acetonitrile: continuous gradient from 75/25 to 55/45 over the course of 120 nm; product was eluted at 65/35) yielded pure 2,2′-(1,1′-(8,8′-(butane-1,4-diylbis(sulfanediyl))bis(octane-8,1-diyl))bis(3-(2-amino-2-oxoacetyl)-2-methyl-1H-indole-4,1-diyl))bis(oxy)diacetic acid (ILY-V-23), (70 mg, 8%).

¹H NMR ((CD₃)₂CS, 300 MHz): δ 7.70 (br s, 2H, NH₂), 7.35 (br s, 2H, NH₂), 7.08 (m, 4H, H-6+H-5), 6.49 (d, 2H, J=7.51 Hz, H-7), 4.57 (s, 4H, H-10), 4.12 (t, 4H, J=7.2 Hz, H-14), 2.51 (s, 6H, H-9), 2.44 (m, 8H, S—CH₂), 1.65 (m, 4H, CH₂), 1.54 (m, 4H, CH₂), 1.46 (m, 4H, CH₂), 1.26 (m, 16H, CH₂).

¹³C NMR ((CD₃)₂CS, 75.5 MHz): δ 189.9 (12), 171.4 (11), 169.2 (13), 152.5 (4), 144.2 (1), 137.8 (8), 123.4 (3), 116.7 (6), 110.7 (5), 104.5 (7+2), 67.8 (10), 43.6 (14), 31.7 (CH₂), 31.3 (CH₂), 29.9 (CH₂), 29.7 (CH₂), 29.3 (CH₂), 29.2 (CH₂), 28.8 (CH₂), 26.9 (CH₂), 25.8 (CH₂), 11.9 (9).

MS (ESI, MeOH): m/z 917.4 [M+Na]⁺.

### Example 11.14

### COMPOUND 5-44

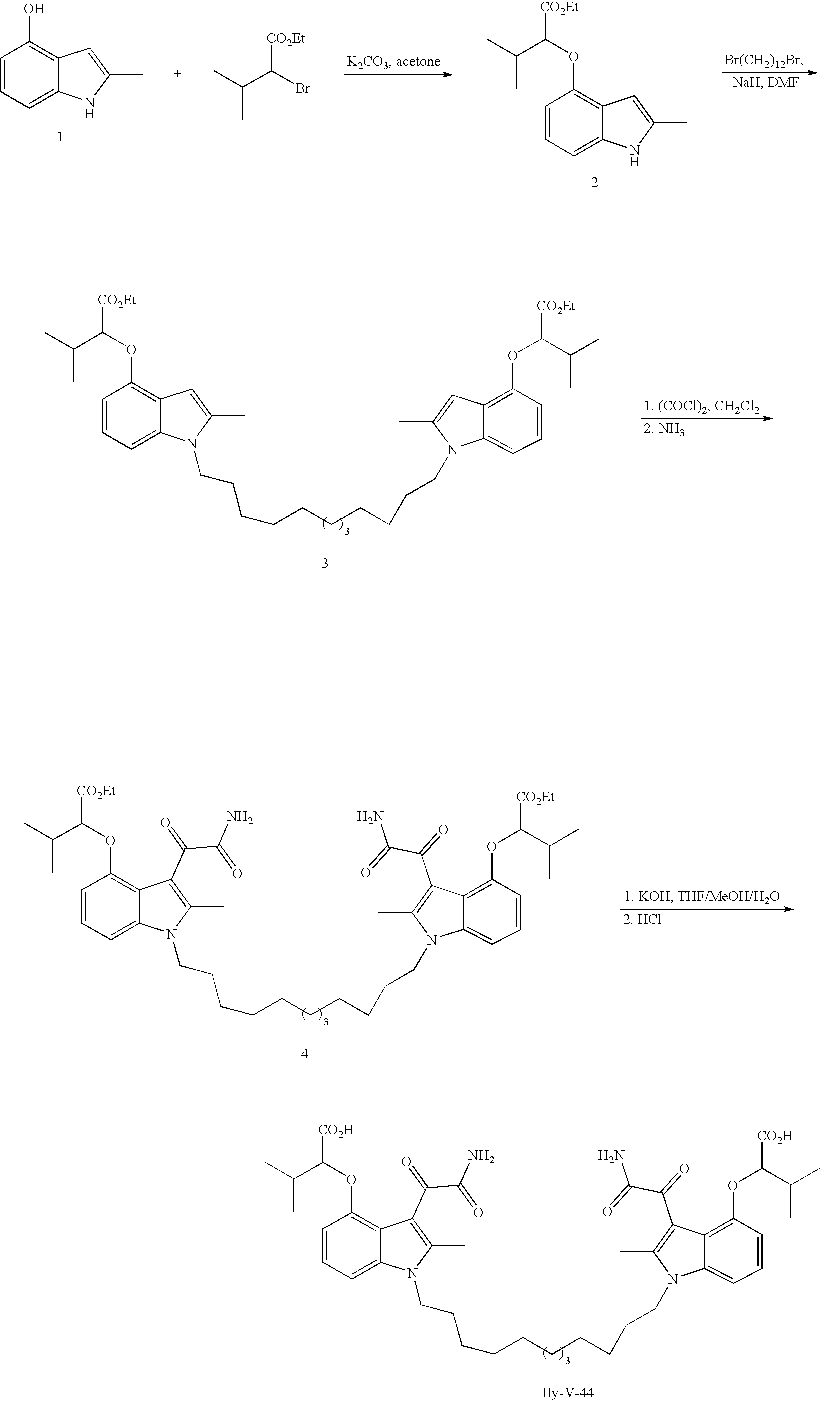

2-(3-Aminooxalyl-1-{12-[3-aminooxalyl-4-(1-ethoxycarbonyl-2-methyl-propoxy)-2-methyl-indol-1-yl]-dodecyl}-2-methyl-1H-indol-4-yloxy)-3-methyl-butyric acid ethyl ester (4): To a solution of intermediate 3 (0.20 g, 0.278 mmole) in anhydrous dichloromethane (20 mL) oxalyl chloride (0.035 g, 0.278 mmole) in anhydrous dichloromethane (20 mL) was added dropwise at 0° C. The mixture was stirred for 1 h. Ammonia was bubbled through the mixture for 20 minutes and stirred for 1 h. The reaction mixture was evaporated. The residue was purified by column chromatography (10:1 CHCl₃:MeOH) to afford intermediate (4) as a yellow solid. Yield: 0.212 g, 91%

2-(3-Aminooxalyl-1-{12-[3-aminooxalyl-4-(1-carboxy-2-methyl-propoxy)-2-methyl-indol-1-yl]-dodecyl}-2-methyl-1H-indol-4-yloxy)-3-methyl-butyric acid (IIy-V-44): A solution of intermediate 4 (100 mg, 0.12 mmol) in THF/CH₃OH/H₂O (1:1:1, 3 mL:3 mL:3 mL) was stirred with 2.2 equivalent of KOH for 4 hr at room temperature. The solution was evaporated and resulting residue was neutralized with 5% HCl at 0° C. The resulting solid was collected by filtration and washed with water and then hexane to afford IIy-V44 as a yellow solid. Yield: 0.067 g, 72%. ¹H NMR (400 MHz, DMSO-d₆) δ, ppm: 12.51(brs, 2H), 8.02 (brs, 2H), 7.61 (brs, 2H), 7.11-7.14(m, 4H), 6.42(d, 2H), 4.42 (d, 2H), 4.16(t, 4H), 2.41 (s,6H), 2.23(m, 2H), 1.62(m, 4H), 1.20-1.32 (m, 16H), 1.07(d, 6H), 0.96(d, 6H) ppm. ES-MS: m/z=803.12(M+1).

### Example 11.15

### COMPOUND (5-41)

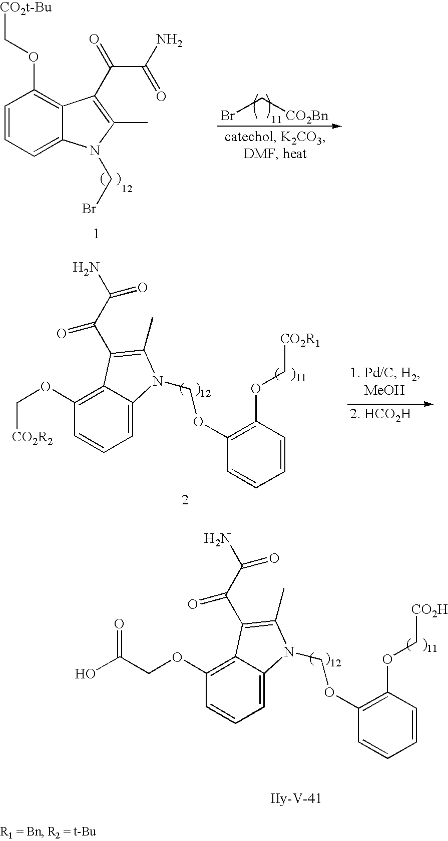

12-{2-[12-(3-Aminooxalyl-4-tert-butoxycarbonylmethoxy-2-methyl-indol-1-yl)-dodecyloxy]-phenoxy}-dodecanoic acid benzyl ester (2): A mixture of [3-aminooxalyl-1-(12-bromo-dodecyl)-2-methyl-1H-indol-4-yloxy]-acetic acid tert-butyl ester (1) (0.654 g, 1.12 mmol), 12-bromobenzyldodecetate (0.416 g, 1.12 mmol), catechol (0.098 g, 0.89 mmol) and K₂CO₃ (2.0 g, excess) were heated in dry DMF (10 mL) at 55° C. for 12 h under N₂. The mixture was cooled and concentrated to dryness. The syrup was suspended in CH₂Cl₂ (15 mL) and filtered through celite. The filtrate was washed with water (10×2 mL) and dried (Na₂SO₄). The solvent was removed under reduced pressure, and the residue was chromatographed on a silica gel column eluting with 80% ethyl acetate in hexane to afford intermediate 2 as an off-white solid. Yield: 0.18 g, 18%.

12-{2-[12-(3-Aminooxalyl-4-carboxymethoxy-2-methyl-indol-1-yl)-dodecyloxy]-phenoxy}-dodecanoic acid (IIy-V-41): Compound 2 (0.12 g, 0.133 mmol) was hydrogenated in presence of Pd—C (10%, 0.01 g) in MeOH (10 mL) for 1 h, then filtered through celite. The filtrate was concentrated to provide colorless syrup. It was then dissolved in aqueous HCO₂H (88%, 2 mL) and the mixture was stirred at room temperature for 6 h. The mixture was concentrated to dryness under high vacuum and co-evaporated with water (2×2 mL). The flask containing the gummy material was then transferred to freeze dryer and was under high vacuum overnight to afford the title compound IIy-V-41 as a white powder. Yield: 0.8 g, 79%. ¹H NMR: (DMSO-d₆), δ, ppm: (5-37-191) δ 7.70 (bs, 3H), 7.40 (bs, 3H), 7.20-7.05 (m, 2H), 6.95-6.80 (m, 4H), 4.62 (s, 2H), 4.18 (t, 2H), 3.83 (t, 4H), 2.47 (s, 3H), 2.09 (t, 2H), 1.70-1.05 (m, 54H). ES-MS: m/z=751.2 (M+1)

### Example 11.16

### COMPOUND (5-36)

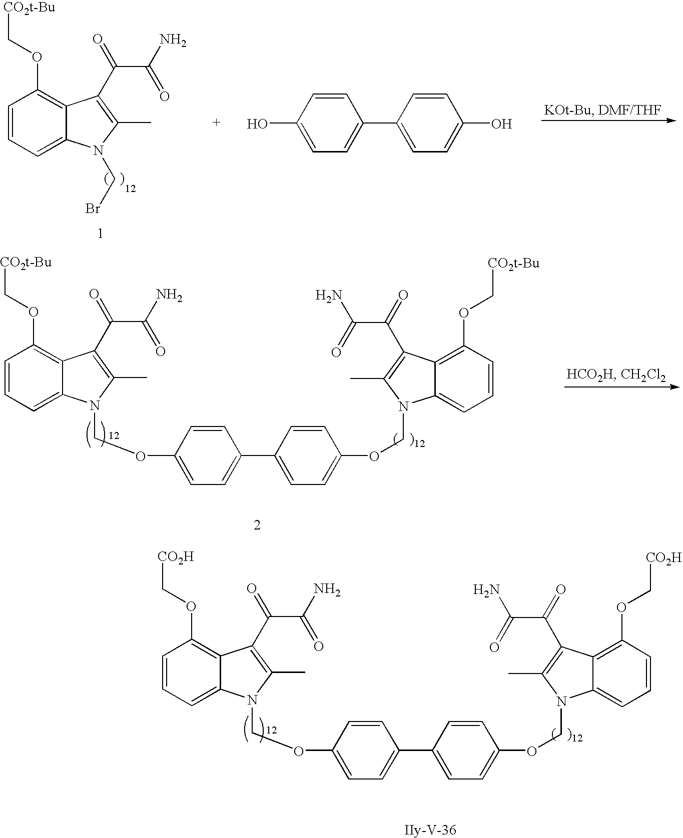

[3-Aminooxalyl-1-(12-{4′-[12-(3-aminooxalyl-4-tert-butoxycarbonylmethoxy-2-methyl-indol-1-yl)-dodecyloxy]-biphenyl-4-yloxy}-dodecyl)-2-methyl-1H-indol-4-yloxy]-acetic acid tert-butyl ester (2): To a solution of 4,4′-dihydroxybiphenyl (0.18 g, 0.966 mmole) in DMF (4 mL) potassium tert-butoxide (1M in THF, 2.12 mL, 2.12 mmole) was added dropwise. The mixture was stirred at 0° C. for 20 minutes. A solution of [3-aminooxalyl-1-(12-bromo-dodecyl)-2-methyl-1H-indol-4-yloxy]-acetic acid tert-butyl ester (1) (1.1 g, 1.89 mmole) in DMF/THF (10 mL/5 mL) was added rapidly to the mixture. The mixture was stirred at 0° C. for 10 h. The reaction was quenched at 0° C. with ammonium chloride solution (20 mL), diluted with water (10 mL) and extracted with ethyl acetate (3×20 mL). The organic layer was separated, washed with brine, dried over sodium sulphate and concentrated. The residue was purified by column chromatography (2:1 CHCl₃:EtOAc) to afford intermediate (2) as a golden brown semi solid. Yield: 0.157 g, 14%.

[3-Aminooxalyl-1-(12-{4′-[12-(3-aminooxalyl-4-carboxymethoxy-2-methyl-indol-1-yl)-dodecyloxy]-biphenyl-4-yloxy}-dodecyl)-2-methyl-1H-indol-4-yloxy]-acetic acid (IIy-V-36): To a solution of intermediate (2) (0.125 g, 0.105 mmole) in dichloromethane, 90% formic acid (35 mL) was added. The mixture was stirred at room temperature for 8 h. The reaction mixture was evaporated and the residue was stirred in diethyl ether (30 mL). The formed solid was collected by filtration and dried under high vacuum to afford IIy-V-36 as a solid. Yield: 0.076 g, 68%. ¹H NMR (400 MHz, DMSO-d₆) 6, ppm: 7.72 (s, 2H), 7.50 (s, 4H), 7.40 (s, 2H), 7.15-7.05 (m, 4H), 6.95 (d, 4H), 6.50 (d, 2H), 4.62 (s, 4H), 4.17 (m, 4H), 3.97 (m, 4H), 2.55 (s, 6H), 1.75-1.60 (m, 8H), 1.45-1.20 (m, 32H). ES-MS: m/z=1070.33 (M−1).

### Example 11.17

### COMPOUND (5-37)

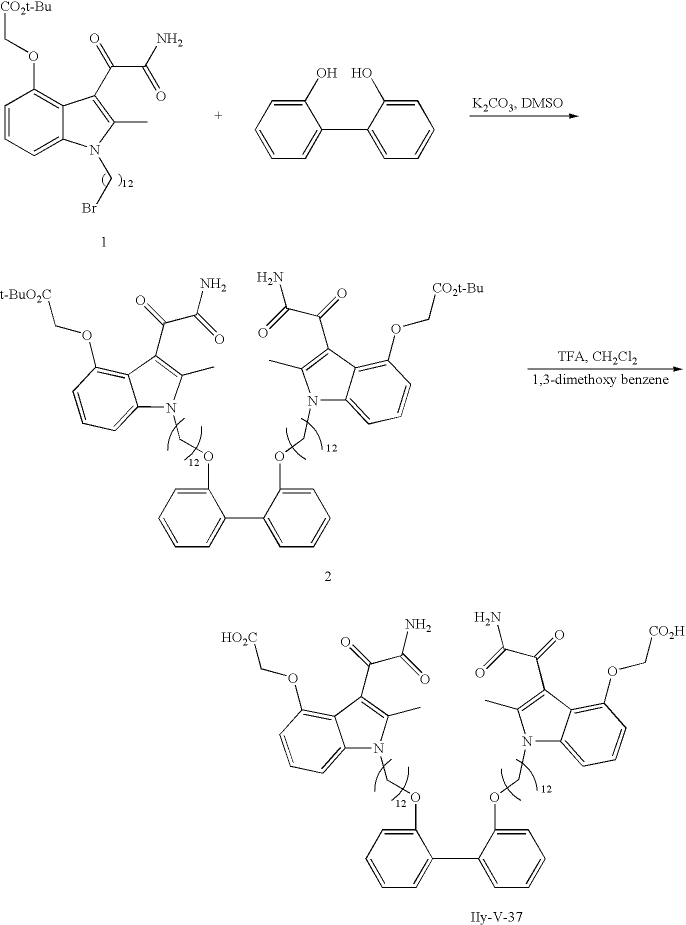

[3-Aminooxalyl-1-(12-{2′-[12-(3-aminooxalyl-4-tert-butoxycarbonylmethoxy-2-methyl-indol-1-yl)-dodecyloxy]-biphenyl-2-yloxy}-dodecyl)-2-methyl-1H-indol-4-yloxy]-acetic acid tert-butyl ester (2): A solution of intermediate 1 (32.62 g, 56.28 mmol) in dimethyl sulfoxide (300 mL) was prepared. To the mixture 2,2′-hydroxybiphenyl (3.52 g, 18.76 mmol) and potassium carbonate (26.45 g, 0.187 mole) was added. The mixture was stirred at 55° C. for 18 h. The reaction mixture was diluted with ethyl acetate (1 L) and washed with ammonium chloride solution (3×1 L). The organic layer was separated, dried with magnesium sulphate and concentrated. The residue was purified by column chromatography (1:1 CHCl₃:EtOAc) affording intermediate 2 as a yellow solid. Yield: 17.63 g, 80%.

[3-Aminooxalyl-1-(12-{2′-[12-(3-aminooxalyl-4-carboxymethoxy-2-methyl-indol-1-yl)-dodecyloxy]-biphenyl-2-yloxy}-dodecyl)-2-methyl-1H-indol-4-yloxy]-acetic acid (IIy-V-37): A solution of intermediate 2 (1.0 g, 0.846 mmol) in anhydrous dichloromethane (40 mL) was prepared. To the mixture, 1,3-dimethoxybenzene (0.25 mL, 1.71 mmol) and trifluoroacetic acid (3 mL) were added. The mixture was stirred at room temperature for 3 h. The solvent was evaporated and the residue was stirred in diethyl ether (50 mL) for 30 minutes. The solid was collected by filtration, washed with diethyl ether and dried to afford IIy-V-37 as a green solid. Yield: 0.8 g, 88%. ¹H NMR (400 MHz, DMSO-d₆) δ, ppm: 12.85 (br, 2H), 7.70 (s, 2H), 7.50 (d, 4H), 7.38 (s, 2H), 7.18-7.05 (m, 4H), 6.95 (d, 4H), 6.55 (d, 2H), 4.62 (s, 4H), 4.20-4.05 (m, 4H), 4.00-3.90 (m, 4H), 2.55 (s, 6H), 1.78-1.60 (m, 8H), 1.40-1.15 (m, 32H). ES-MS: m/z=1071.26 (M+1).

### Example 12

### IN-VITRO ASSAY FOR THE INHIBITION OF HUMAN, MOUSE AND PORCINE PHOSPHOLIPASE A₂

In this example, a fluorimetric assay procedure was used to evaluate the indole and indole-related compounds of the invention as inhibitors of group 1B phospholipase A₂ (PLA₂) from human, mouse and porcine. A description of this assay is found in articles: Leslie, C C and Gelb, M H (2004) Methods in Molecular Biology “Assaying phospholipase A2 activity”, 284:229-242; Singer, A G, et al. (2002) Journal of Biological Chemistry “Interfacial kinetic and binding properties of the complete set of human and mouse groups I, II, V, X, and XII secreted phospholipases A2”, 277:48535-48549, which are incorporated herein as references.

In general, this assay used a phosphatidylmethanol substrate with a pyrene fluorophore on the terminal end of the sn-2 fatty acyl chain. Without being bound by theory, close proximity of the pyrenes from neighboring phospholipids in a phospholipid vesicle caused the spectral properties to change relative to that of monomeric pyrene. Bovine serum albumin was present in the aqueous phase and captured the pyrene fatty acid when it is liberated from the glycerol backbone owing to the PLA2-catalyzed reaction. However, a potent inhibitor can inhibit the liberation of pyrene fatty acid from the glycerol backbone. Hence, such features allow for a sensitive PLA2 inhibition assay by monitoring the fluorescence of albumin-bound pyrene fatty acid. The effect of a given inhibitor and inhibitor concentration on human, mouse and porcine phospholipase was determined.

Recombinant human and mouse group 1B PLA2 were cloned and expressed in E. coli as insoluble inclusion bodies. The inclusion bodies were isolated and purified by sonicating cell pellet in lysis buffer (50 mM Tris-HCl pH 7.0, 250 mM NaCl, 0.5% Triton 100), centrifugation at 12,000×g, and washing three times in washing buffer (20 mM Tris-HCl pH 7.0, 250 mM NaCl, 0.5% Triton 100). Then the inclusion bodies were dissolved in dissolving buffer (50 mM Tris-HCl pH 7.0, 250 mM NaCl, 6 M Guanidine-HCl, 1 mM DTT) and dialyzed four times against 10 volumes of refolding buffer (20 mM Tris-HCl pH 7.0, 250 mM NaCl, 0.5M Guanidine-HCl, 5% (w/w) Glycerol, 2 mM reduced glutathione and 0.4 mM oxidized glutathione) at 4° C. The correctly refolded proteins were concentrated using Amicon Stirred cell under nitrogen pressure (<70 psi) and dialyzed against 10 volumes of 50 mM Tris-HCl pH 7.0, 250 mM NaCl and 5% (w/w) glycerol. Human and mouse group 1B PLA2 were further purified by High S ion exchange and gel filtration columns.

The following reagents and equipments were obtained from commercial vendors:
1. Porcine group 1B phospholipase A₂
2. 1-hexadecanoyl-2-(1-pyrenedecanoyl)-sn-glycero-3-phosphomethanol (PPyrPM)
3. Bovine serum albumin (BSA, fatty acid free)
4. 2-Amino-2-(hydroxymethyl)-1,3-propanediol, hydrochloride (Tris-HCl)
5. Calcium chloride
6. Potassium chloride
7. Solvents: DMSO, toluene, isopropanol, ethanol
8. Molecular Devices SPECTRAmax microplate spectrofluorometer
9. Costar 96 well black wall/clear bottom plate

The following reagents were prepared:
10. PPyrPM stock solution (1 mg/ml) in toluene:isopropanol (1:1)
11. ILY104 inhibitor stock solution (10 mM) in DMSO
12. 3% (w/v) bovine serum albumin (BSA)
13. Stock buffer: 50 mM Tris-HCl, pH 8.0, 50 mM KCl and 1 mM CaCl₂

The following procedure was performed to evaluate the inhibitory potency of the evaluated compounds.
14. An assay buffer was prepared by adding 3 ml 3% BSA to 47 ml stock buffer.
15. Solution A was prepared by adding serially diluted inhibitors to the assay buffer. Inhibitors were three-fold diluted in stock buffer in a series of 8 from 15 uM.
16. Solution B was prepared by adding human, mouse or porcine PLA₂ to the assay buffer. This solution was prepared immediately before use to minimize enzyme activity loss.
17. Solution C was prepared by adding 30 ul PPyrPM stock solution to 90 ul ethanol, and then all 120 ul of PPyrPM solution was transferred drop-wise over approximately 1 min to the continuously stirring 8.82 ml assay buffer to form a final concentration of 4.2 uM PPyrPM vesicle solution.
18. The SPECTRAmax microplate spectrofluorometer was set at 37° C.
19. 100 ul of solution A was added to each inhibition assay well of a costar 96 well black wall/clear bottom plate
20. 100 ul of solution B was added to each inhibition assay well of a costar 96 well black wall/clear bottom plate.
21. 100 ul of solution C was added to each inhibition assay well of a costar 96 well black wall/clear bottom plate.
22. The plate was incubated inside the spectrofluorometer chamber for 3 min.
23. The fluorescence was read using an excitation of 342 nm and an emission of 395 nm.

Evaluated compounds were tested in duplicate and their values were averaged to plot the inhibition curve and calculate the IC50. Compared to uninhibited controls, lower fluorescent signal at an emission of 395 nm in test reactions evidenced inhibition of PLA₂. Although the final concentration of compounds in reactions typically ranged from 15 uM to 0.007 uM, the more potent inhibitors were diluted to a much lower concentration. Compounds initially found to be active were repeated to confirm their inhibitory activity. The IC50 was calculated using the BioDataFit 1.02 (Four Parameter Model) software package. The equation used to generate the inhibition curve fit is:

$${y}_{j} = β + \frac{α - β}{1 + exp ( - κ ( log ( {x}_{j} ) - γ ) )}$$

wherein: α is the value of the upper asymptote; β is the value of the lower asymptote; κ is a scaling factor; γ is a factor that locates the x-ordinate of the point of inflection at

$$exp [ \frac{κγ - log ( \frac{1 + κ}{κ - 1} )}{κ} ]$$

with constraints α, β, κ, γ>0, β<α, and β<γ<α. In experiments in which the IC 50 value was not reached at concentrations of 15 uM of the compound under test, the % inhibition at 15 uM was reported.

The results of the inhibition assay for pancreas secreted human, mouse and porcine group 1B PLA₂ by the evaluated compounds are summarized in Table 4.

TABLE 4
Inhibition of pancreas secreted human, mouse and porcine PLA₂

| | Compound | | ILYPSA IC50 (μM) | | | ILYPSA % inhibition at 15 μM | | |
|---|---|---|---|---|---|---|---|---|
| structure | ID | MW | hps PLA₂ | pps PLA₂ | mps PLA₂ | hps PLA₂ | pps PLA₂ | mps PLA₂ |

| | | | | | |
|---|---|---|---|---|---|
| 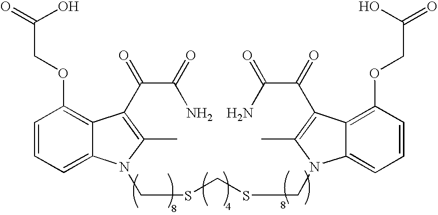 | ILY-V-23 (5-23) | 894.39 | 2.16 | 1.12 | 0.55 |
| 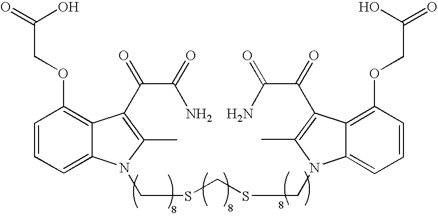 | ILY-V-24 (5-24) | 991.24 | 0.54 | 0.8 | 1.05 |
| 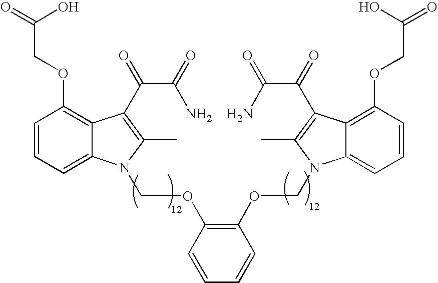 | ILY-V-27 (5-27) | 995.21 | 0.15 | 0.15 | 0.2 |
| 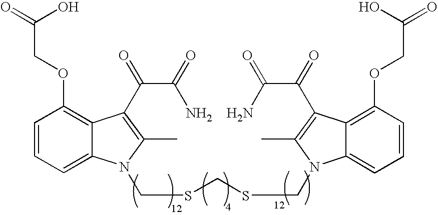 | ILY-V-25 (5-25) | 1007.35 | 0.15 | 0.19 | 0.35 |
| 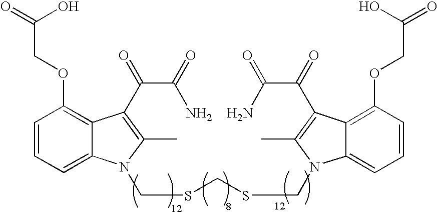 | ILY-V-26 (5-26) | 1063.45 | 0.24 | 0.26 | 0.34 |
| 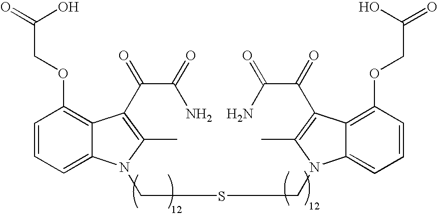 | IL-V-29 (5-29) | 919.18 | 0.46 | 0.53 | 0.79 |
| 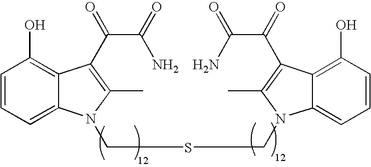 | ILY-V-35 (5-35) | 803.1 | 1.52 | 2.09 | 3.65 |
| 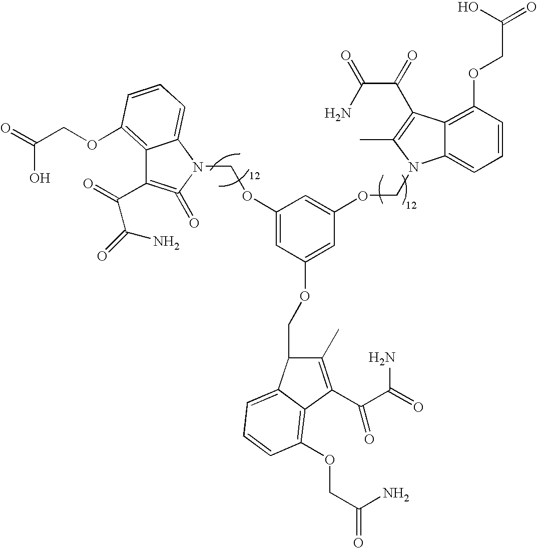 | ILY-V-32 (5-32) | 1453.75 | 0.06 | 0.09 | 0.13 |
| 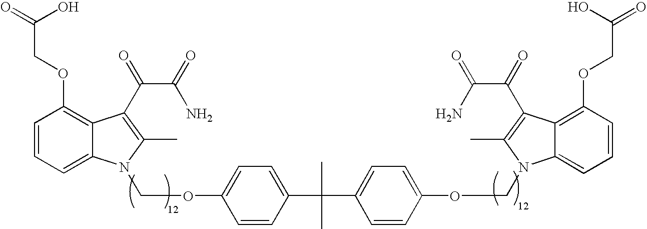 | ILY-V-30 | 1113.41 | 0.43 | <0.02 | 0.19 |
| 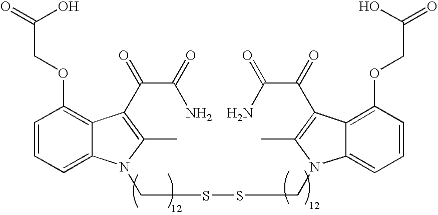 | ILY-V-28 (5-28) | 951.2 | 0.6 | 0.73 | 1 |
| 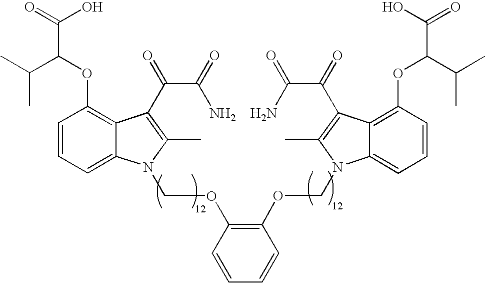 | IL-V-33 (5-33) | 1079.37 | 0.22 | 0.24 | 0.2 |
| 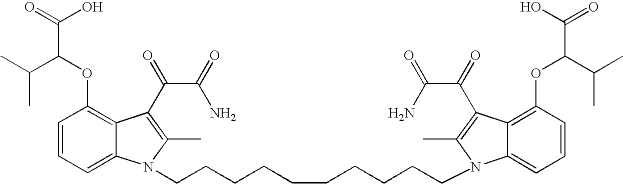 | ILY-V-44 (5-44) | 802 | 0.28 | 0.05 | 0.48 |
| 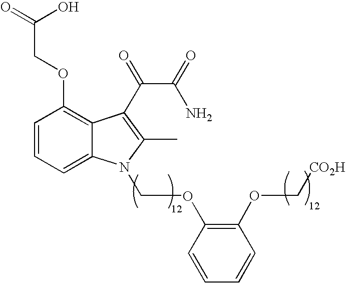 | ILY-V-41 (5-41) | 751 | 1.54 | 1.14 | 1.6 |
| 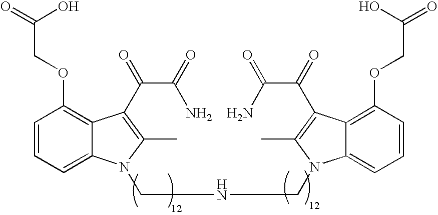 | ILY-V-45 (5-45) | 902.15 | 0.12 | 0.04 | 0.07 |
| 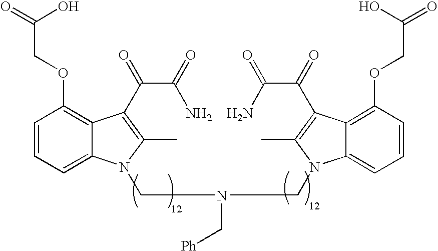 | ILY-V-31 (5-31) | 952.25 | 0.1 | 0.02 | 0.03 |
| 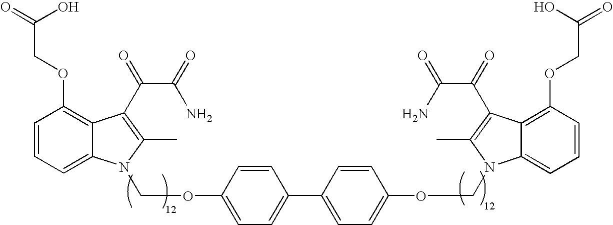 | ILY-V-36 (5-36) | 1071.3 | 1.25 | 0.44 | 0.92 |
| 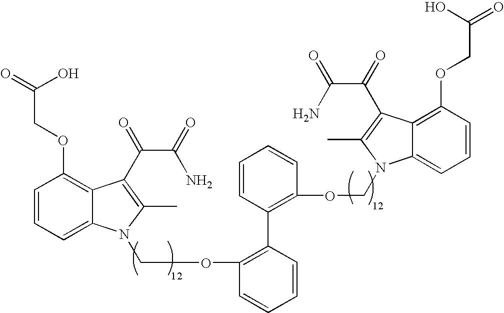 | ILY-V-37 (5-37) | 1071.3 | 0.27 | 0.2 | 0.23 |

These data demonstrate that the multivalent indole and indole related compounds of the invention are active for inhibiting phospholipase A2.

### Example 13

### BIOAVAILABILITY OF MULTIVALENT INDOLE OR INDOLE RELATED COMPOUNDS

This example shows that the multivalent indole or indole related compounds of the invention that are phospholipase inhibitors (See Example 12) are not significantly absorbed (i.e., are substantially lumen-localized).

Bioavailability was determined for Compound 5-24 (ILY-V-24) as follows. Generally, the bioavailability was calculated by comparing a timecourse of the concentration of the test compound in the serum of mice after an intravenous (IV) dosing, versus the timecourse of the concentration following an oral dosing of the test compound. The IV dose was ˜3 mg/kg, the oral dose was ˜30 mg/kg.

Materials. The following materials were used for preparing the oral and IV formulations:

| Material | Vendor | Cat or Lot# |
|---|---|---|
| ILY-V-24 | Ilypsa | |
| CMC Medium Viscosity USP | Sigma-Aldrich | C9481 |
| Ethanol ESP/NF | Sigma-Aldrich | 493538 |
| PEG 300 - Ultra Grade | Sigma-Aldrich | 90878 |
| PEG 400 - Ultra Grade | Sigma-Aldrich | 91893 |
| Tween-80 Ultra. 100 ML | Sigma-Aldrich | P8074 |
| DMSO Hybri-MAX | Sigma-Aldrich | D2650 |

Oral Formulation. The oral formulation was prepared as follows. To sterile flask, 90 ml of sterile Milli-Q water was added. 9 ml of PEG-400 was added (final concentration of 9%). 50 ul of Tween-80 was added (final concentration of 0.05%). 0.9 g of CMC was weighed and added (final concentration of 0.9% w/v). A clean stir-bar was added and the CMC was dissolved effectively by stirring overnight at RT. ˜30 mg of test compound was weighed into a 40 ml glass vial. ˜10 ml of oral formulation was added (final test article concentration of 3 mg/ml). The vial was vortexed and then sonicated in a warming, sonicating bath for 30 minutes. At the end of this period, much of the test article was in suspension, but some particulates were observed in the bottom of the vial. This preparation was sonicated for a further hour, checked for precipitating particulates before dosing and was kept well mixed during dosing.

Intravenous (IV) Formulation. The intravenous formulation was prepared as follows. To sterile flask, 60 ml of sterile Milli-Q water was added. 30 ml of PEG-300 was added (final concentration of 30%). 5 ml of EtOH was added (final concentration of 5%). This resulted in the IV formulation, minus DMSO. The test compound was dissolved as follows. ˜3 mg of test article was weighed into a 10 ml glass vial and ˜500 ul of DMSO was added. ˜9.5 ml of the above IV formulation (minus DMSO) was added to a 40 ml glass vial, for a final concentration of 3 mg test article in 10 ml IV formulation (containing 5% DMSO). The formulation was vortexed before dosing.

Study Design. The bioavailability study was designed as follows. Three groups (N=18, 24 or 3) of male CD-1 Mus Musculus mice were used for each study. On study day 0, all the animals were weighed, dosages were calculated and the animals were dosed by oral route (PO) or (IV) as outlined below in Table 5. PO formulation was sonicated in a warm sonication bath for an hour prior to dosing. IV formulation was vortexed for 5 mins immediately prior to dosing. Blood for plasma (0.5 mL/sample) was collected at specified time intervals and placed into labeled Eppendorf® tubes with Potassium-EDTA as an anti-coagulant, centrifuged and pipetted off into labeled Eppendorf® tubes (for at least 0.2 ml plasma) and frozen at −80° C.

TABLE 5
Bioavailability Study Details

| | | Group | Time Points | Mice Per |
|---|---|---|---|---|
| Compound | Dose | Number | (hr) | time point |
| Test Article | PO | 1 | 0.5, 1, 2, 4, 8, 24 | 3 |
| | 30 mg/kg | | | |
| | 10 ml/kg | | | |
| Test Article | IV | 2 | 5 min, 15 min, | 3 |
| | 3 mg/kg | | 0.5, 1, 2, 4, 8, 24 | |
| | 10 ml/kg | | | |
| None | None | 3 | N/A | 3 |

Calculations. Bioavailability was calculated as follows. Individual doses were calculated based on an average of body weights taken on the day of dosing. Serum concentrations of test compound, as well as the actual concentration of dosing solutions, were measured using 2-dimensional Mass Spectrophotometry after Liquid Chromatography (LC MS/MS). Methods were optimized for each test article and internal standards were used in all cases.

The maximum concentration (Cₘₐₓ) in plasma and the time to reach maximum concentration (Tₘₐₓ) were obtained by visual inspection of the raw data. Pharmacokinetic parameters were calculated using GraphPad Prism 4.0 software and included half-life (t_{1/2}) and area under the concentration-time curve from time 0 to the last time point (AUC₀₋ₜ). Visual inspection of the data shows in all cases that AUC₀₋ₜ was very similar in the case of all test articles to the area under the concentration-time curve from 0 to infinity (AUC_{0-∞})

Bioavailability (% F) was calculated using the following relationship:
% F=(AUC_{0-t,oral}/AUC_{0-t,iv})×(Doseᵢᵥ/Doseₒᵣₐₗ)×100
where: % F is bioavailability; AUC₀₋ₜ is area under the concentration-time curve at the last measurable time-point, and IV refers to intravenous.

Results. The bioavailability for Compound 5-24 (ILY-V-24) was determined to be about 4-8%.

### Example 14

### SYNTHESIS OF C4-ACIDIC INDOLE AND INDOLE RELATED COMPOUNDS, AND IN-VITRO ASSAY FOR CERTAIN OF SUCH COMPOUNDS FOR THE INHIBITION OF HUMAN, MOUSE AND PORCINE PHOSPHOLIPASE A₂

In this example, various preferred indole and indole-related compounds having specific C4-acidic moieties are prepared.

### Example 14.1

### COMPOUND 4-20

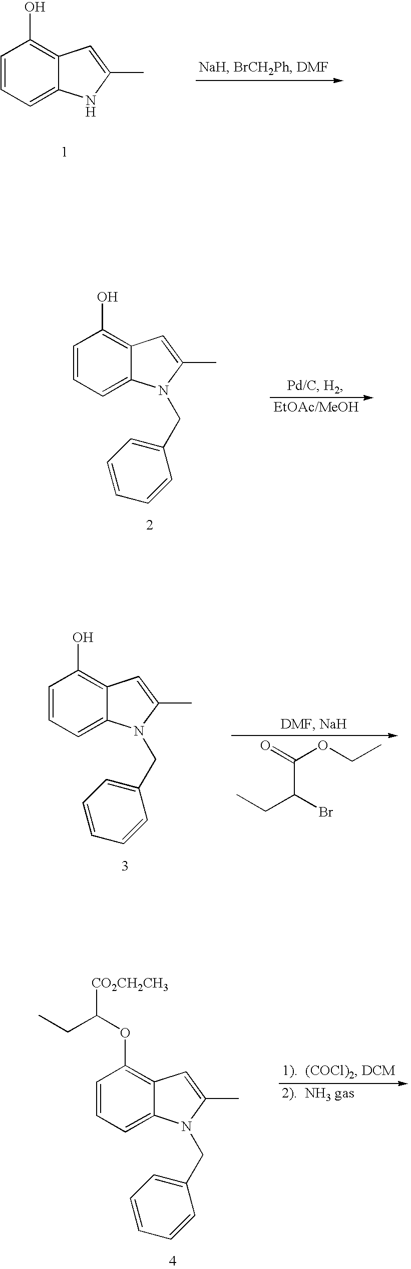

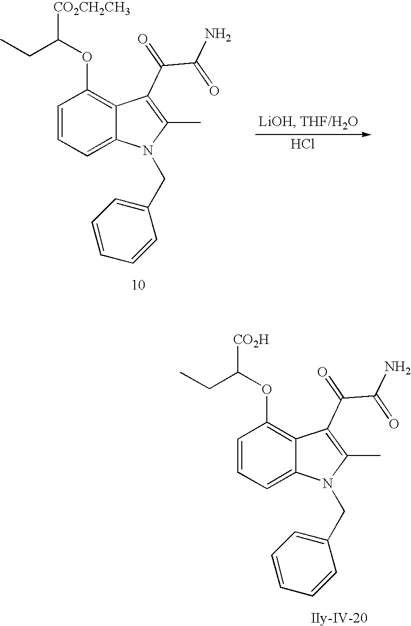

1-Benzyl-4-benzyloxy-2-methyl-1H-indole 2: 4-hydroxy-2-methyl indole 1 (50 g, 0.339 mole) was dissolved in anhydrous DMF (1 L). To the mixture sodium hydride 60% in mineral oil (27.9 g, 0.697 mole) was added. The mixture was left to stir at rt. for 1 h. To the mixture benzyl bromide (82.7 mL, 0.697 mole) was added drop-wise. The mixture was left to stir at room temperature for 18 h. The reaction was diluted with ethyl acetate (4 L) and washed with water (5×500 mL) then brine (1 L). The organic layer was separated and dried with magnesium sulphate and concentrated. The orange oily residue was purified by column chromatography (6:1 Hexane:EtOAc) to afford 86 g (72%) of 2 as an yellow oil.

1-Benzyl-2-methyl-1H-indol-4-ol 3: 1-Benzyl-4-benzyloxy-2-methyl-1H-indole 2 (86 g, 0.263 mole) was dissolved with ethyl acetate (1.5 L) and methanol (300 mL). To the mixture 10% Pd/C wet (18 g) was added to the solution. The reaction was then subjected to H₂ gas passed through a mercury bubbler at room temperature and 1 atm. The mixture was left to stir for 6 h. The reaction mixture was filtered through Celite and concentrated. The residue was purified by column chromatography (3:1 Hexane:EtOAc) to afford 3 (30 g, 49%) as a cream solid.

2-(1-Benzyl-2-methyl-1H-1indol-4-yloxy)-butric acid ethyl ester 4: 1-Benzyl-2-methyl-1H-indol-4-ol 3 (0.5 g 2.1 mmole) was dissolved in anhydrous dimethylformamide (100 mL). To the solution sodium hydride 60% in mineral oil (0.11 g 2.73 mmole) was added. The mixture was stirred at room temperature for 1 h. To the mixture ethyl-2-bromobutyrate (0.4 mL, 2.73 mmole) was added. The mixture was stirred at room temperature for 72 h. The reaction was diluted with ethyl acetate (500 mL) and washed with H₂O (5×100 mL) and brine (1×100 mL). The organic layer was separated, dried with magnesium sulfate and concentrated. The residue was purified by column chromatography (8:1 Hexane:EtOAc) to afford 4 (0.32 g, 43%) as an orange oil.

2-(3-Aminooxalyl-1-benzyl-2-methyl-1H-indol-4-yloxy)-butyric acid ethyl ester 10: To a solution of oxalyl chloride (0.1 mL, 1.09 mmole) in anhydrous dichloromethane (100 mL) a solution of 2-(1-Benzyl-2-methyl-1H-1indol-4-yloxy)-butric acid ethyl ester 4 (0.32 g, 0.914 mmole) in anhydrous dichloromethane (100 mL) was added drop-wise. The mixture was left to stir at room temperature for 1 h. NH₃ gas was then bubbled through the solution for 30 minutes. The mixture was left to stir at room temperature for 18 h. The dichloromethane was evaporated and the residue was dissolved in ethyl acetate 300 mL) and washed with H₂O (2×300 mL) and brine (1×300 mL). The organic layer was separated, dried with magnesium sulfate and concentrated to afford 10 (0.35 g, 91%) as a green solid.

2-(3-Aminooxalyl-1-benzyl-2-methyl-1H-indol-4-yloxy)-butyric acid IIy-IV-20: 2-(3-Aminooxalyl-1-benzyl-2-methyl-1H-indol-4-yloxy)-butyric acid ethyl ester 10 (0.2 g, 0.477 mmole) was dissolved in THF:H₂O 4:1 (10 mL). To the mixture lithium hydroxide monohydrate (0.024 g, 0.573 mmole) was added. The mixture was left to stir at room temperature for 18 h. The mixture was acidified to pH 3 with 2M HCl. The resulting precipitate was collected by filtration and washed with water and dried to afford IIy-IV-20 (0.043 g, 23%) as a yellow solid.

Ref: 04-090-249.1: ¹H NMR (DMSO) δ 12.63 (s, broad, 1H), 7.95 (s, 1H), 7.55 (s, broad, 1H), 7.35-7.00 (m, 7H), 6.47 (d, 1H), 5.50 (s, 2H), 3.4 (m, 1H), 2.50 (s, 3H), 1.95 (m, 2H), 1.00 (m, 3H). MS (ES+) 395.02

### Example 14.2

### COMPOUND 4-24

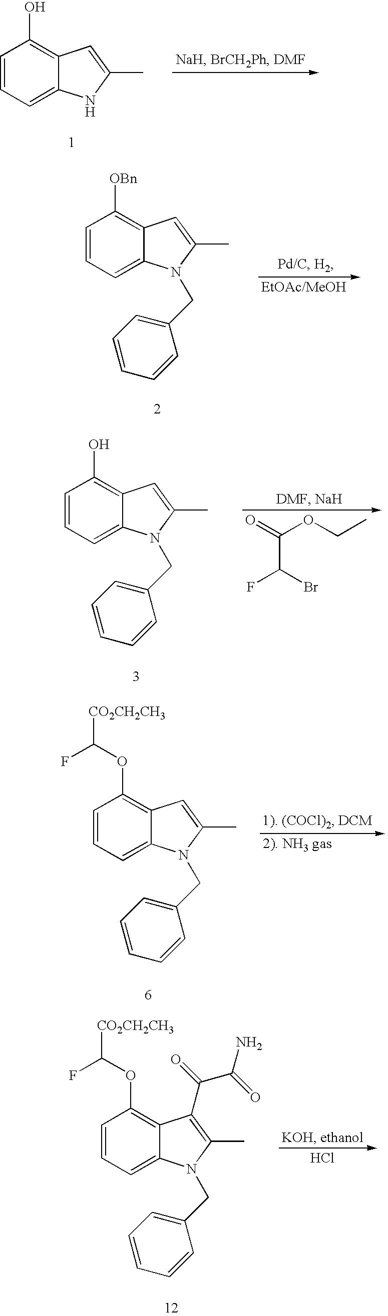

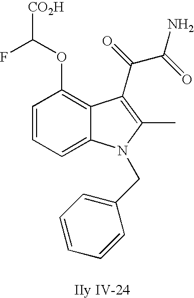

1-Benzyl-4-benzyloxy-2-methyl-1H-indole 2: 4-hydroxy-2-methyl indole 1 (50 g, 0.339 mole) was dissolved in anhydrous DMF (1 L). To the mixture sodium hydride 60% in mineral oil (27.9 g, 0.697 mole) was added. The mixture was left to stir at rt. for 1 h. To the mixture benzyl bromide (82.7 mL, 0.697 mole) was added drop-wise. The mixture was left to stir at room temperature for 18 h. The reaction was diluted with ethyl acetate (4 L) and washed with water (5×500 mL) then brine (1 L). The organic layer was separated and dried with magnesium sulphate and concentrated. The orange oily residue was purified by column chromatography (6:1 Hexane:EtOAc) to afford 86 g (72%) of 2 as an yellow oil.

1-Benzyl-2-methyl-1H-indol-4-ol 3: 1-Benzyl-4-benzyloxy-2-methyl-1H-indole 2 (86 g, 0.263 mole) was dissolved with ethyl acetate (1.5 L) and methanol (300 mL). To the mixture 10% Pd/C wet (18 g) was added to the solution. The reaction was then subjected to H₂ gas passed through a mercury bubbler at room temperature and 1 atm. The mixture was left to stir for 6 h. The reaction mixture was filtered through Celite and concentrated. The residue was purified by column chromatography (3:1 Hexane:EtOAc) to afford 3 (30 g, 49%) as a cream solid.

(1-Benzyl-2-methyl-1H-indol-4-yloxy)-fluoro-acetic acid ethyl ester 6: 1-Benzyl-2-methyl-1H-indol-4-ol 3 (0.3 g 1.26 mmole) was dissolved in anhydrous dimethylformamide (50 mL). To the solution sodium hydride 60% in mineral oil (66 mg 1.65 mmole) was added. The mixture was stirred at room temperature for 1 h. To the mixture ethyl-2-bromofluoroacetate (0.2 mL, 1.65 mmole) was added. The mixture was stirred at room temperature for 18 h. The reaction was diluted with ethyl acetate (500 mL) and washed with H₂O (5×100 mL) and brine (1×100 mL). The organic layer was separated, dried with magnesium sulfate and concentrated. The residue was purified by column chromatography (6:1 Hexane:EtOAc) to afford 6 (0.14 g, 32%) as an yellow oil.

(3-Aminooxalyl-1-benzyl-2-methyl-1H-indol-4-yloxy)-fluoro-acetic acid ethyl ester 12: To a solution of oxalyl chloride (0.042 mL, 0.478 mmole) was diluted in anhydrous dichloromethane (25 mL). To the solution (1-Benzyl-2-methyl-1H-indol-4-yloxy)-fluoro-acetic acid ethyl ester 6 (0.14 g, 0.398 mmole) in anhydrous dichloromethane (25 mL) was added drop-wise. The mixture was left to stir at room temperature for 2 h. NH₃ gas was then bubbled through the solution for 30 minutes. The mixture was left to stir at room temperature for 1.5 h. The dichloromethane was evaporated and the residue was dissolved in ethyl acetate 300 mL) and washed with H₂O (2×300 mL) and brine (1×300 mL). The organic layer was separated, dried with magnesium sulfate and concentrated. The residue was purified by preparative TLC (3:1 EtOAc:Hex) to afford 12 (0.02 g, 12%) as a yellow solid. Also isolated as a polar product (Rf ˜0.2)

(3-Aminooxalyl-1-benzyl-2-methyl-1H-indol-4-yloxy)-fluoro-acetic acid IIy-IV-24: (3-Aminooxalyl-1-benzyl-2-methyl-1H-indol-4-yloxy)-fluoro-acetic acid ethyl ester 12 (0.06 g, 0.145 mmole) was dissolved in anhydrous ethanol (10 mL). To the mixture 0.5054 N potassium hydroxide solution was added (0.15 mL, 0.152 mmole). The mixture was left to stir at room temperature for 30 min. The ethanol was evaporated and H₂O (5 mL) was added. The solution was acidified to pH 2 with 0.5 M HCl. The mixture was extracted with ethyl acetate (100 mL). The organic was washed with H₂O (100 mL), separated, dried with magnesium sulfate and concentrated to afford IIy-IV-24 (5 mg, 9%) as a green solid.

Ref: 04-090-287.1: ¹H NMR (DMSO) δ 7.70 (s, 1H), 7.40-6.90 (m, 9H), 6.20 (d, 1H), 5.50 (s, 2H), 2.50 (s, 3H). MS (ES+) 384.94

### Example 14.3

### COMPOUND 4-22

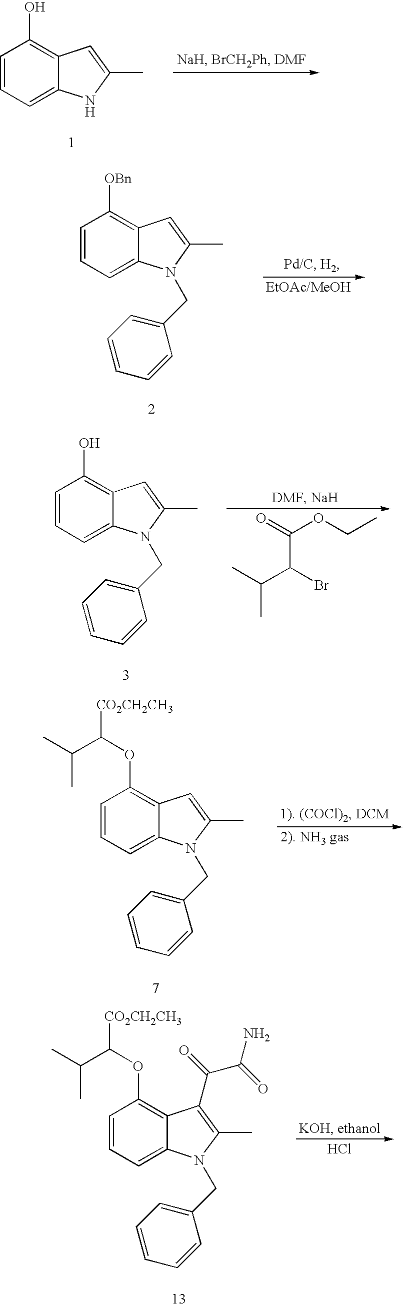

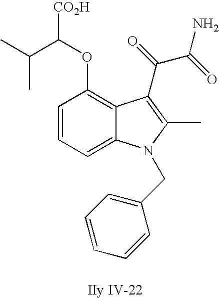

1-Benzyl-4-benzyloxy-2-methyl-1H-indole 2: 4-hydroxy-2-methyl indole 1 (50 g, 0.339 mole) was dissolved in anhydrous DMF (1 L). To the mixture sodium hydride 60% in mineral oil (27.9 g, 0.697 mole) was added. The mixture was left to stir at rt. for 1 h. To the mixture benzyl bromide (82.7 mL, 0.697 mole) was added drop-wise. The mixture was left to stir at room temperature for 18 h. The reaction was diluted with ethyl acetate (4 L) and washed with water (5×500 mL) then brine (1 L). The organic layer was separated and dried with magnesium sulphate and concentrated. The orange oily residue was purified by column chromatography (6:1 Hexane:EtOAc) to afford 86 g (72%) of 2 as an yellow oil.

1-Benzyl-2-methyl-1H-indol-4-ol 3: 1-Benzyl-4-benzyloxy-2-methyl-1H-indole 2 (86 g, 0.263 mole) was dissolved with ethyl acetate (1.5 L) and methanol (300 mL). To the mixture 10% Pd/C wet (18 g) was added to the solution. The reaction was then subjected to H₂ gas passed through a mercury bubbler at room temperature and 1 atm. The mixture was left to stir for 6 h. The reaction mixture was filtered through Celite and concentrated. The residue was purified by column chromatography (3:1 Hexane:EtOAc) to afford 3 (30 g, 49%) as a cream solid.

2-(1-Benzyl-2-methyl-1H-indol-4-yloxy)-3-methyl-butyric acid ethyl ester 7: 1-Benzyl-2-methyl-1H-indol-4-ol 3 (0.3 g 1.26 mmole) was dissolved in anhydrous dimethylformamide (20 mL). To the solution sodium hydride 60% in mineral oil (66 mg 1.65 mmole) was added. The mixture was stirred at room temperature for 1 h. To the mixture ethyl-2-bromoisovalerate (0.344 mL, 1.65 mmole) was added. The mixture was stirred at room temperature for 18 h. The reaction was diluted with ethyl acetate (300 mL) and washed with H₂O (4×100 mL) and brine (1×100 mL). The organic layer was separated, dried with magnesium sulfate and concentrated. The residue was purified by column chromatography (10:1 Hexane:EtOAc) to afford a 1:1 mixture of 7:ethyl-2-bromoisovalerate. Further purification by column chromatography (10:1 Hexane:EtOAc) afforded 7 (0.09 g, 19%) as a yellow oil.

2-(3-Aminooxalyl-1-benzyl-2-methyl-1H-indol-yloxy)-3-methyl-butyric acid ethyl ester 13: 2-(1-Benzyl-2-methyl-1H-indol-4-yloxy)-3-methyl-butyric acid ethyl ester 7 (0.09 g, 0.247 mmole) was dissolved in anhydrous dichloromethane (50 mL). To the solution oxalyl chloride (0.026 mL, 0.296 mmole) was added. The mixture was left to stir at room temperature for 1 h. NH₃ gas was then bubbled through the solution for 30 minutes. The mixture was left to stir at room temperature for 1 h. The dichloromethane was evaporated and the residue was dissolved in ethyl acetate (200 mL) and washed with H₂O (3×200 mL) and brine (1×300 mL). The organic layer was separated, dried with magnesium sulfate and concentrated to afford 13 (0.23 g, >100%) as a yellow solid (contained inorganic salt). The material was used in next step without further purification.

2-(3-Aminooxalyl-1-benzyl-2-methyl-1H-indol-4-yloxy)-3-methyl-butyric acid IIy-IV-22: 2-(3-Aminooxalyl-1-benzyl-2-methyl-1H-indol-yloxy)-3-methyl-butyric acid ethyl ester 13 (0.15 g, 0345 mmole) was dissolved in anhydrous ethanol (10 mL). To the mixture 0.5054 N potassium hydroxide solution (0.4 mL, 0.403 mmole) was added. The mixture was left to stir at room temperature for 72 h. The reaction mixture was evaporated under high vacuum. The residue was dissolved in H₂O (5 mL) and acidified with 2M HCl. The mixture was left to stir for 30 min. The precipitate was collected by filtration washed and with H₂O to afford IIy-IV-22 (0.03 g, 21%) as a yellow solid.

Ref: 04-090-270.1: ¹H NMR (DMSO) δ 12.60 (s, broad, 1H), 8.00 (s, 1H), 7.60 (s, 1H), 7.40-7.00 (m, 7H), 6.50 (d, 1H), 5.50 (s, 2H), 4.47 (d, 1H), 2.42 (s, 3H), 2.30 (m, 1H), 1.10-0.90 (m, 6H). MS (ES+) 409.00

### Example 14.4

### Compound 4-33

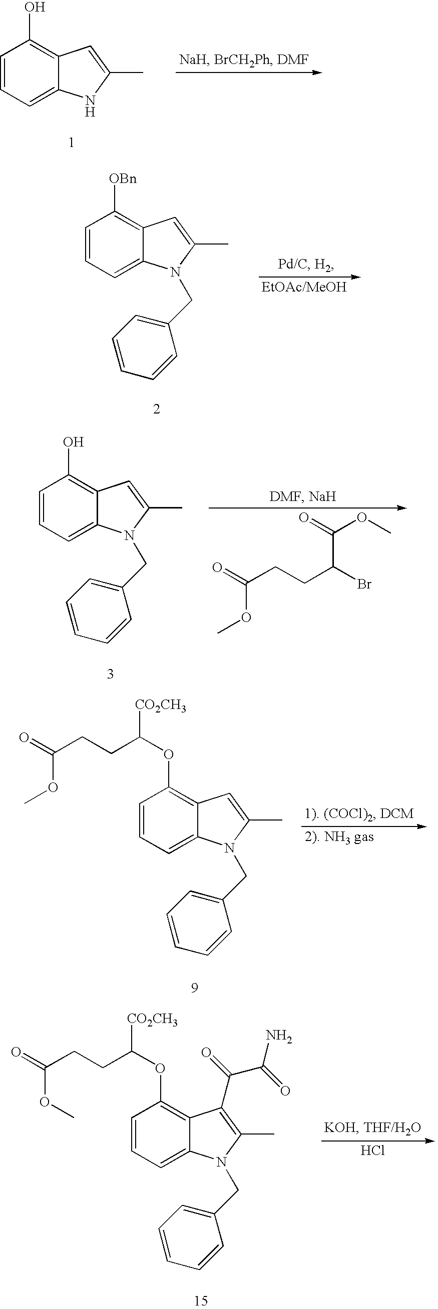

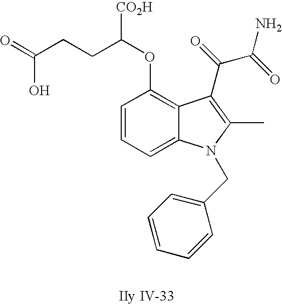

1-Benzyl-4-benzyloxy-2-methyl-1H-indole 2: 4-hydroxy-2-methyl indole 1 (50 g, 0.339 mole) was dissolved in anhydrous DMF (1 L). To the mixture sodium hydride 60% in mineral oil (27.9 g, 0.697 mole) was added. The mixture was left to stir at rt. for 1 h. To the mixture benzyl bromide (82.7 mL, 0.697 mole) was added drop-wise. The mixture was left to stir at room temperature for 18 h. The reaction was diluted with ethyl acetate (4 L) and washed with water (5×500 mL) then brine (1 L). The organic layer was separated and dried with magnesium sulphate and concentrated. The orange oily residue was purified by column chromatography (6:1 Hexane:EtOAc) to afford 86 g (72%) of 2 as an yellow oil.

1-Benzyl-2-methyl-1H-indol-4-ol 3: 1-Benzyl-4-benzyloxy-2-methyl-1H-indole 2 (86 g, 0.263 mole) was dissolved with ethyl acetate (1.5 L) and methanol (300 mL). To the mixture 10% Pd/C wet (18 g) was added to the solution. The reaction was then subjected to H₂ gas passed through a mercury bubbler at room temperature and 1 atm. The mixture was left to stir for 6 h. The reaction mixture was filtered through Celite and concentrated. The residue was purified by column chromatography (3:1 Hexane:EtOAc) to afford 3 (30 g, 49%) as a cream solid.

2-(1-Benzyl-2-methyl-1H-indol-4-yloxy)-pentanedioic acid 1-methyl ester 5-methyl ester 9: 1-Benzyl-2-methyl-1H-indol-4-ol 3 (0.3 g 1.26 mmole) was dissolved in anhydrous dimethylformamide (20 mL). To the solution sodium hydride 60% in mineral oil (66 mg 1.65 mmole) was added. The mixture was stirred at room temperature for 1 h. To the mixture dimethyl-2-bromoglutarate (0.3 mL, 1.25 mmole) was added. The mixture was stirred at room temperature for 18 h. The reaction was diluted with ethyl acetate (300 mL) and washed with H₂O (4×100 mL) and brine (1×100 mL). The organic layer was separated, dried with magnesium sulfate and concentrated. The residue was purified by column chromatography (6:1 Hexane:EtOAc) to afford 9 (0.49 g, 97%) as a white solid.

2-(3-Aminooxalyl-1-benzyl-2-methyl-1H-indol-4-yloxy)-pentanedioic acid dimethyl ester 15: 2-(1-Benzyl-2-methyl-1H-indol-4-yloxy)-pentanedioic acid 1-methyl ester 5-methyl ester 9 (0.15 g, 0.38 mmole) was dissolved in anhydrous dichloromethane (50 mL). To the solution oxalyl chloride (0.037 mL, 0.396 mmole) was added. The mixture was left to stir at room temperature for 2 h. NH₃ gas was then bubbled through the solution for 30 minutes. The mixture was left to stir at room temperature for 1 h. The dichloromethane was evaporated and the residue was dissolved in ethyl acetate (200 mL) and washed with H₂O (3×200 mL) and brine (1×300 mL). The organic layer was separated, dried with magnesium sulfate and concentrated to afford 15 (0.17 g, 96%) as a yellow solid.

2-(3-Aminooxalyl-1-benzyl-2-methyl-1H-indol-4-yloxy)-pentanedioic acid IIy-IV-33: 2-(3-Aminooxalyl-1-benzyl-2-methyl-1H-indol-4-yloxy)-pentanedioic acid dimethyl ester 15 (0.08 g, 0.172 mmole) was dissolved in THF:H₂O 4:1 (10 mL). To the mixture 0.5054 N potassium hydroxide solution (0.48 mL, 0.495 mmole) was added. The mixture was left to stir at room temperature for 72 h. The reaction mixture was evaporated to dryness, then dissolved in H₂O (5 mL) and acidified to pH 4 with 2M HCl. The resulting precipitate was collected by filtration and dried to afford IIy-IV-33 (0.03 g, 40%) as a yellow solid.

Ref: 04-090-288.2: ¹H NMR (DMSO) δ 8.40 (s, broad, 1H), 7.92 (s, 1H), 7.40-7.20 (m, 3H), 7.10-6.90 (m, 4H), 6.40 (d, 1H), 5.45 (s, 2H), 4.20 (t, broad, 1H), 2.50 (s, 3H), 2.40-1.90 (m, 4H). MS (ES−) 436.98 (ES+) 460.91 (M+Na⁺).

### Example 14.5

### COMPOUND 4-32

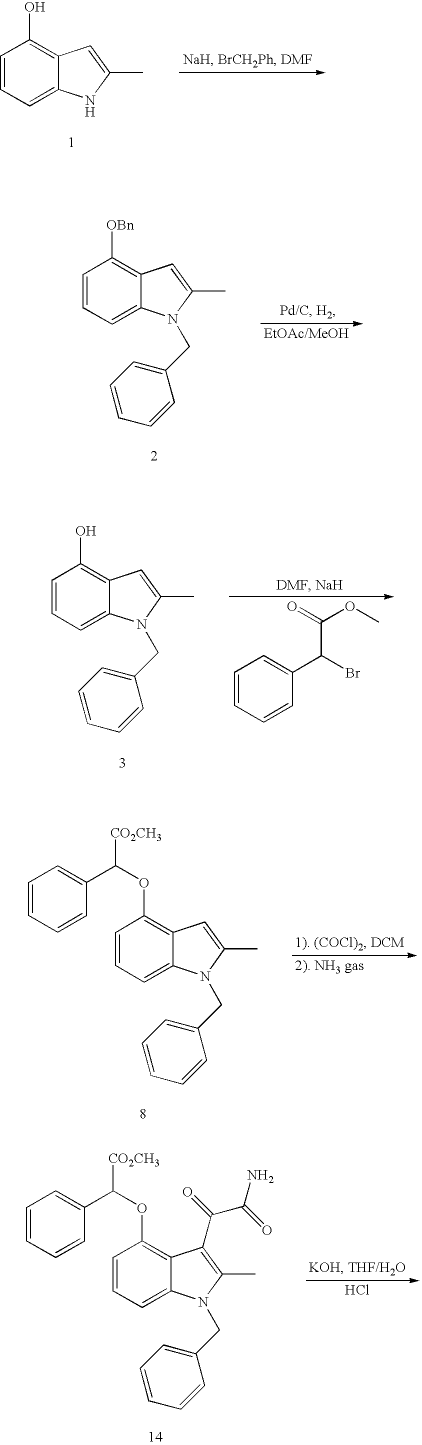

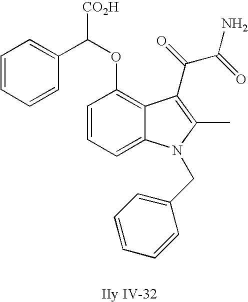

1-Benzyl-4-benzyloxy-2-methyl-1H-indole 2: 4-hydroxy-2-methyl indole 1 (50 g, 0.339 mole) was dissolved in anhydrous DMF (1 L). To the mixture sodium hydride 60% in mineral oil (27.9 g, 0.697 mole) was added. The mixture was left to stir at rt. for 1 h. To the mixture benzyl bromide (82.7 mL, 0.697 mole) was added drop-wise. The mixture was left to stir at room temperature for 18 h. The reaction was diluted with ethyl acetate (4 L) and washed with water (5×500 mL) then brine (1 L). The organic layer was separated and dried with magnesium sulphate and concentrated. The orange oily residue was purified by column chromatography (6:1 Hexane:EtOAc) to afford 86 g (72%) of 2 as an yellow oil.

1-Benzyl-2-methyl-1H-indol-4-ol 3: 1-Benzyl-4-benzyloxy-2-methyl-1H-indole 2 (86 g, 0.263 mole) was dissolved with ethyl acetate (1.5 L) and methanol (300 mL). To the mixture 10% Pd/C wet (18 g) was added to the solution. The reaction was then subjected to H₂ gas passed through a mercury bubbler at room temperature and 1 atm. The mixture was left to stir for 6 h. The reaction mixture was filtered through Celite and concentrated. The residue was purified by column chromatography (3:1 Hexane:EtOAc) to afford 3 (30 g, 49%) as a cream solid.

(1-Benzyl-2-methyl-1H-indol-4-yloxy)-phenyl-acetic acid methyl ester 8: 1-Benzyl-2-methyl-1H-indol-4-ol 3 (0.3 g 1.26 mmole) was dissolved in anhydrous dimethylformamide (20 mL). To the solution sodium hydride 60% in mineral oil (66 mg 1.65 mmole) was added. The mixture was stirred at room temperature for 1 h. To the mixture bromo-phenyl-acetic acid methyl ester (0.24 mL, 1.512 mmole) was added. The mixture was stirred at room temperature for 18 h. The reaction was diluted with ethyl acetate (300 mL) and washed with H₂O (4×100 mL) and brine (1×100 mL). The organic layer was separated, dried with magnesium sulfate and concentrated. The residue was purified by column chromatography (10:1 Hexane:EtOAc) to afford 8 (0.3 g, 62%) as a white solid.

(3-Aminooxalyl-1-benzyl-2-methyl-1H-indol-4-yloxy)-2-phenyl-acetic acid methyl ester 14: (1-Benzyl-2-methyl-1H-indol-4-yloxy)-phenyl-acetic acid methyl ester 8 (0.15 g, 0.389 mmole) was dissolved in anhydrous dichloromethane (50 mL). To the solution oxalyl chloride (0.04 mL, 0.428 mmole) was added. The mixture was left to stir at room temperature for 2 h. NH₃ gas was then bubbled through the solution for 30 minutes. The mixture was left to stir at room temperature for 1 h. The dichloromethane was evaporated and the residue was dissolved in ethyl acetate (200 mL) and washed with H₂O (3×200 mL) and brine (1×300 mL). The organic layer was separated, dried with magnesium sulfate and concentrated to afford 14 (0.15 g, 85%) as a yellow solid.

(3-Aminooxalyl-1-benzyl-2-methyl-1H-indol-4-yloxy)-phenyl-acetic acid IIy-IV-32: (3-Aminooxalyl-1-benzyl-2-methyl-1H-indol-4-yloxy)-2-phenyl-acetic acid methyl ester 14 (0.15 g, 0.33 mmole) was dissolved in THF:H₂O 4:1 (10 mL). To the mixture 0.5054 N potassium hydroxide solution (0.48 mL, 0.495 mmole) was added. The mixture was left to stir at room temperature for 18 h. The reaction mixture was evaporated to dryness. The residue was dissolved in H₂O (5 mL) and acidified to pH 4 with 2M HCl. The resulting precipitate was collected by filtration washed with H₂O and dried to afford IIy-IV-32 (0.08 g, 55%) as a yellow solid.

Ref: 04-090-281.1: ¹H NMR (DMSO) δ 12.90 (s, broad, 1H), 7.90 (s, broad, 1H), 7.65 (d, 2H), 7.50-7.00 (m, 11H), 6.60 (d, 1H), 6.85 (s, 1H), 5.50 (s, 2H), 2.45 (s, 3H). MS (ES+) 443.02

### Examples 14.6a, 14.7a, 14.8-14.10

### COMPOUNDS 4-47, 4-46, 4-8, 4-1 AND 4-19

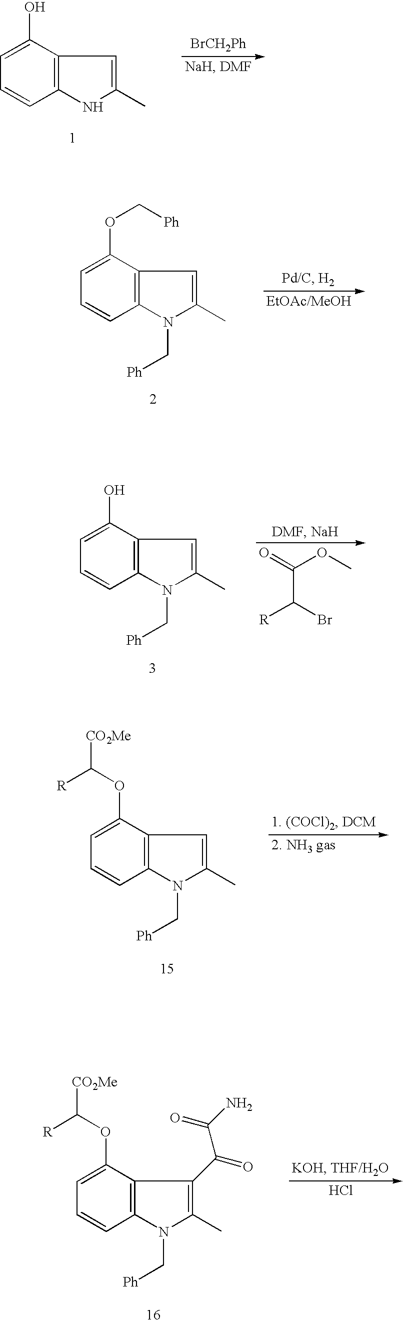

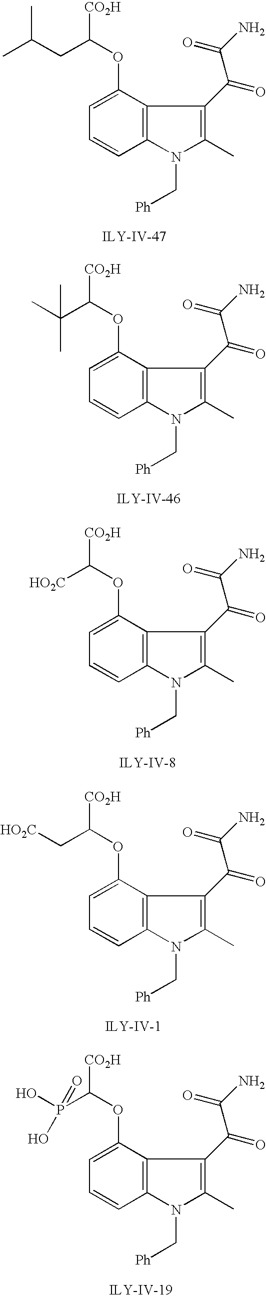

2-(3-(2-amino-2-oxoacetyl)-1-benzyl-2-methyl-1H-indol-4-yloxy)-4-methylpentanoic acid (ILY-IV-47); 2-(3-(2-amino-2-oxoacetyl)-1-benzyl-2-methyl-1H-indol-4-yl oxy)-3,3-dimethylbutanoic acid (ILY-IV-46); 2-(3-(2-amino-2-oxoacetyl)-1-benzyl-2-methyl-1H-indol-4-yloxy)malonic acid (ILY-IV-8); 2-(3-(2-amino-2-oxo acetyl)-1-benzyl-2-methyl-1H-indol-4-yloxy)-2-phosphonoacetic acid (ILY-IV-1); 2-(3-(2-amino-2-oxoacetyl)-1-benzyl-2-methyl-1H-indol-4-yloxy)succinic acid (ILY-IV-19) can be prepared according to the schema shown above and the following description.

Alkylation: 1-Benzyl-2-methyl-1H-indol-4-ol 3 (1 mmole) is dissolved in anhydrous dimethylformamide (20 mL). To the solution, sodium hydride 60% in mineral oil (1.2 mmole) is added. The mixture is stirred at room temperature for 1 h. To the mixture the corresponding bromo-acetic acid methyl ester (1.2 mmole) is added. The mixture is stirred at room temperature for 18 h. The reaction is diluted with ethyl acetate (300 mL) and washed with H₂O (4×100 mL) and brine (1×100 mL). The organic layer is to be separated, dried with magnesium sulfate and concentrated. The residue is purified by column chromatography to afford 15.

Glyoxamidation: The corresponding acetic acid methyl ester 15 (1 mmole) is dissolved in anhydrous dichloromethane (50 mL). To the solution oxalyl chloride (1.1 mmole) is added. The mixture is left to stir at room temperature for 2 h. NH₃ gas is then bubbled through the solution for 30 minutes. The mixture is left to stir at room temperature for 1 h. The dichloromethane is evaporated and the residue is dissolved in ethyl acetate (200 mL) and washed with H₂O (3×200 mL) and brine (1×300 mL). The organic layer is separated, dried with magnesium sulfate and concentrated to afford 16.

Deprotection: Compound 16 (1 mmole) is dissolved in THF:H₂O 4:1 (10 mL). To the mixture 0.5054 N potassium hydroxide solution is added. The mixture is left to stir at room temperature for 18 h. The reaction mixture is evaporated to dryness. The residue is dissolved in H₂O (5 mL) and is acidified to pH 4 with 2M HCl. The resulting precipitate is collected by filtration washed with H₂O and dried to afford IIy-IV-47, IIy-IV-46, IIy-IV-8, IIy-IV-1, and IIy-IV-19.

### Example 14.6b

### COMPOUND 4-47

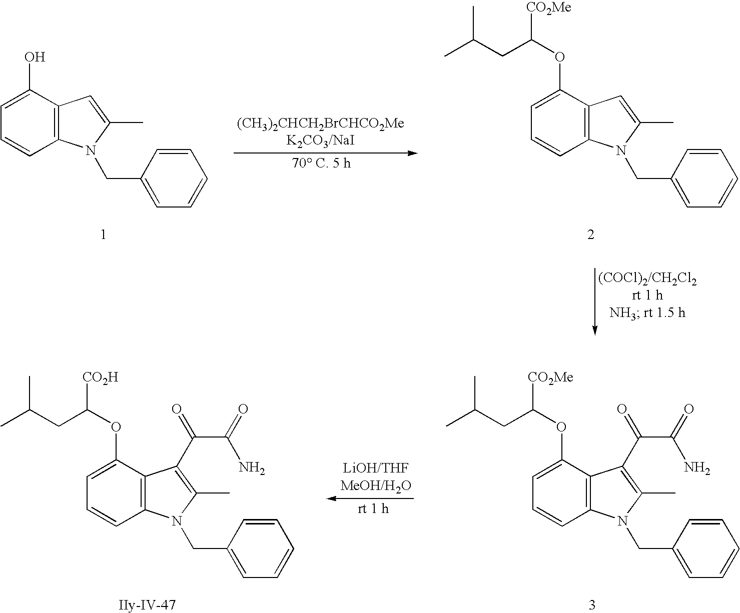

2-(1-Benzyl-2-methyl-1H-indol-4-yloxy)-4-methyl-pentanoic acid methyl ester (2): To a stirred suspension of K₂CO₃ (0.563 g, 4.22 mmol), NaI (0.031 g, 0.21 mmol) and 1-benzyl-2-methyl-1H-indol-4-ol (1) (0.500 g, 2.11 mmol) in dry DMF (15 mL), a solution of (CH₃)₂CHCH₂BrCHCO₂Me (0.66 g, 3.2 mmol) in DMF (5 mL) was added dropwise. The reaction mixture was heated at 70° C. for 7 h, cooled to room temperature and water (30 mL) was added. The mixture was extracted with EtOAc (3×50 mL). The combined organic extracts were washed with water (50 mL), brine (50 mL), dried over Na₂SO₄ and evaporated. Flash chromatography of the residue over silica gel, using 10% EtOAc in hexanes to 20% EtOAc in hexanes, gave product 2 as a pale yellow solid. Yield: 0.54 g (70%).

2-(3-Aminooxalyl-1-benzyl-2-methyl-1H-indol-4-yloxy)-4-methyl-pentanoic acid methyl ester (3): A solution of 2-(1-benzyl-2-methyl-1H-indol-4-yloxy)-4-methyl-pentanoic acid methyl ester (2) (243 mg, 0.671 mmol) in CH₂Cl₂ (10 mL) was prepared. To this mixture, oxalyl chloride (0.075 mL, 0.85 mmol) was added dropwise, and the mixture was stirred at room temperature for 1 h. Ammonia was bubbled through the mixture for 30 minutes and stirred for another 1 h. The reaction mixture was diluted with EtOAc (100 mL), washed with water (50 mL), brine (50 mL), dried over Na₂SO₄ and concentrated. The residue was purified by crystallization from CHCl₃/hexanes (1:1) to afford intermediate (3) as a yellow solid. Yield: 0.220 g (76%).

2-(3-Aminooxalyl-1-benzyl-2-methyl-1H-indol-4-yloxy)-4-methyl-pentanoic acid (IIy-IV-47): To a solution of 2-(3-aminooxalyl-1-benzyl-2-methyl-1H-indol-4-yloxy)-4-methyl-pentanoic acid methyl ester (3) (150 mg, 0.344 mmol) in THF/MeOH/H₂O (5 mL/5 mL/5 mL) lithium hydroxide monohydrate (0.041 g, 1.72 mmol) was added. The reaction mixture was stirred at room temperature for 1 h, evaporated and then acidified (pH=4) with 1 N HCl to form a white precipitate, which was filtered off, washed with water and dried in vacuum to afford product IIy-IV-47 as a yellow solid. Yield: 125 mg (86%). ¹H NMR: 05-056-069 (DMSO-d₆, 400 MHz) δ, ppm: 0.88 (d, 3H), 0.95 (d, 3H), 1.55-1.65 (m, 1H), 1.76-2.04 (m, 2H), 2.45 (s, 3H), 4.70 (m, 1H), 5.48 (s, 2H), 6.54 (d, 1H), 7.00-7.18 (m, 4H), 7.20-7.38 (m, 3H), 7.58 (s, 1H), 8.02 (s, 1H) (COOH not shown). ES-MS: m/z=422.99 (M+1).

### Example 14.7b

### COMPOUND 4-8

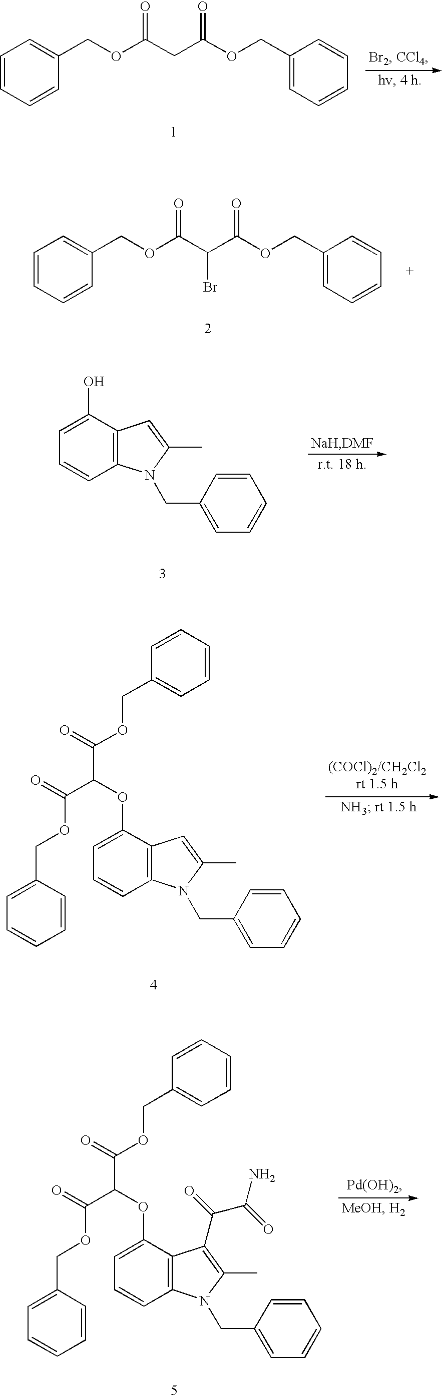

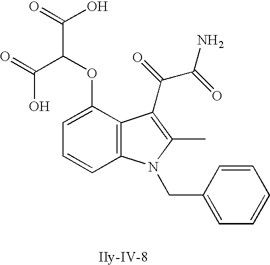

2-Bromo-malonic acid dibenzyl ester (2): To a solution of dibenzyl malonate (9.8 g, 34.46 mmole) in carbon tetrachloride (25 mL), bromine (10.14 g, 63.4 mmole) was added dropwise at room temperature over 4 h. The reaction mixture was irradiated with a 150 W lamp during the addition. The reaction mixture was quenched with water. The organic layer was separated and the aqueous layer was further extracted with dichloromethane (3×30 mL). The organic extracts were combined, washed with sodium hydrogen carbonate solution (3×50 mL) and brine solution 3×50 mL). The organic layer was dried over magnesium sulphate and concentrated. The residue was purified by column chromatography (9:1 Hex:EtOAc) to afford intermediate 2 as an orange oil. Yield 3.8 g, 30%

2-(1-Benzyl-2-methyl-1H-indol-4-yloxy)-malonic acid dibenzyl ester (4): To a solution of 1-benzyl-2-methyl-1H-indol-4-ol (3) (1.0 g, 4.22 mmole) in DMF (30 mL), sodium hydride (0.285 g, 5.48 mmole, 60% in mineral oil) was added. The mixture was stirred at room temperature for 45 minutes. To the reaction mixture a solution of 2-bromo-malonic acid dibenzyl ester (2) (1.9 g, 5.48 mmole) in DMF (20 mL) was added dropwise. The mixture was stirred at room temperature for 18 h. The reaction mixture was diluted with ethyl acetate (50 mL) and washed with water (3×50 mL) and brine (3×50 mL). The organic layer was separated and dried over magnesium sulphate and concentrated. The residue was purified by column chromatography (3:1 Hex:EtOAc) to afford a mixture of starting material (2) and intermediate (4). The crude material was used in the following step without further purification.

2-(3-Aminooxalyl-1-benzyl-2-methyl-1H-indol-4-yloxy)-malonic acid dibenzyl ester (5): To a solution of 2-(1-benzyl-2-methyl-1H-indol-4-yloxy)-malonic acid dibenzyl ester (4) (0.2 g, crude material) in dichloromethane (50 mL), oxalyl chloride (0.1 mL, 1.06 mmole) was added. The mixture was stirred at room temperature for 1.5 h. Ammonia gas was bubbled through the solution for 30 min. Then the mixture was stirred for an additional 1 h. The solvent was evaporated. The residue was dissolved in ethyl acetate (50 mL) and washed with water (3×50 mL) and brine (3×50 mL). The organic layer was separated, dried over magnesium sulphate and concentrated. The residue was purified by preparative TLC (1:1 Hex:EtOAc) to afford intermediate (4) as a yellow solid. Yield: 0.12 g

2-(3-Aminoooxalyl-1-benzyl-2-methyl-1H-indol-4-yloxy)-malonic acid (IIy-IV-8): To a solution of 2-(3-aminooxalyl-1-benzyl-2-methyl-1H-indol-4-yloxy)-malonic acid dibenzyl ester (5) (0.07 g, 0.1206 mmole) in methanol (75 mL), palladium hydroxide (0.017 mg, 50% water wet) was added. Hydrogen was then bubbled through the mixture at 1 atm and room temperature for 30 minutes. The reaction mixture was filtered through Celite and the filtrate was concentrated to afford a yellow solid (0.030 mg). Analysis by ¹H NMR indicated that approximately 30% mono decarboxylation had occurred. ¹H NMR (400 MHz, DMSO-d₆) δ, ppm: 7.47 (brs, 1H), 7.35-6.95 (m, 8H), 6.28 (d, 1H), 5.50 (s, 2H), 4.92 (s, 1H), 2.50 (s, 3H). ES-MS: m/z=410.94 (M+1).

### Example 14.11

### COMPOUND 4-44

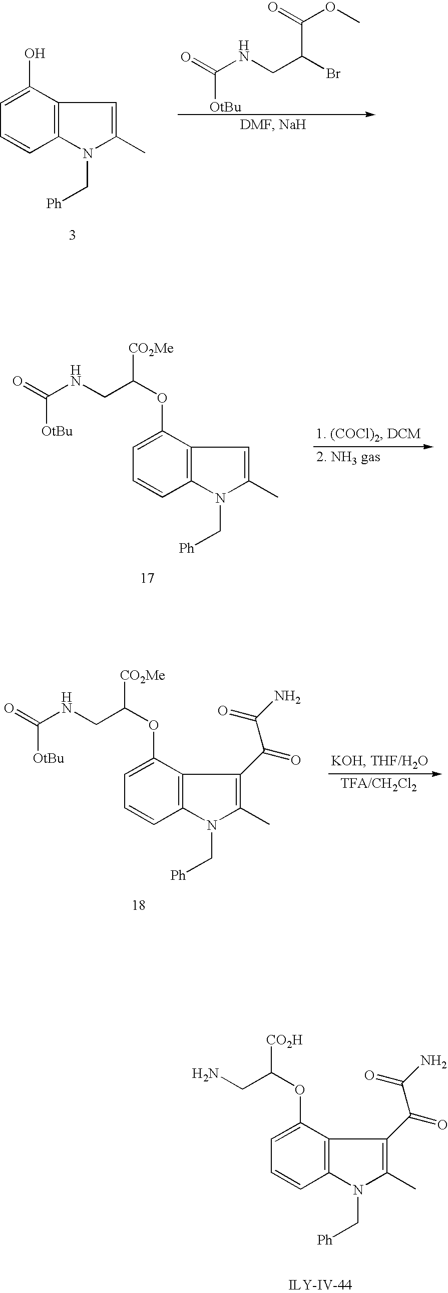

3-amino-2-(3-(2-amino-2-oxoacetyl)-1-benzyl-2-methyl-1H-indol-4-yloxy)propanoic acid (ILY-IV-44) 1-Benzyl-2-methyl-1H-indol-4-ol 3 (1 mmole) is dissolved in anhydrous dimethylformamide (20 mL). To the solution sodium hydride, 60% in mineral oil (1.2 mmole) is added. The mixture is stirred at room temperature for 1 h. To the mixture the corresponding bromo-acetic acid methyl ester (1.2 mmole) is added. The mixture is stirred at room temperature for 18 h. The reaction is diluted with ethyl acetate (300 mL) and is washed with H₂O (4×100 mL) and brine (1×100 mL). The organic layer is separated, dried with magnesium sulfate and concentrated. The residue is purified by column chromatography to afford 17.

The corresponding acetic acid methyl ester 17 (1 mmole) is dissolved in anhydrous dichloromethane (50 mL). To the solution oxalyl chloride (1.1 mmole) is added. The mixture was left to stir at room temperature for 2 h. NH₃ gas is then bubbled through the solution for 30 minutes. The mixture is left to stir at room temperature for 1 h. The dichloromethane is evaporated and the residue is dissolved in ethyl acetate (200 mL) and washed with H₂O (3×200 mL) and brine (1×300 mL). The organic layer is to be separated, dried with magnesium sulfate and concentrated to afford 18.

Compound 18 (1 mmole) is dissolved in THF:H₂O 4:1 (10 mL). To the mixture 0.5054 N potassium hydroxide solution is added. The mixture is left to stir at room temperature for 18 h. The reaction mixture is evaporated to dryness. The dried mixture and 1,3-dimethoxybenzene (7 mmole) in dry dichloromethane (30 mL), at room temperature under nitrogen, is added with trifluoroacetic acid (30 mL). The solution is stirred for 1 h and the solvents evaporated below 25° C. The residue is dissolved in H₂O (5 mL) and acidified to pH 4 with 2M HCl. The resulting precipitate is collected by filtration washed with H₂O and dried to afford IIy-IV-44.

### Example 14.12

### COMPOUND 4-48

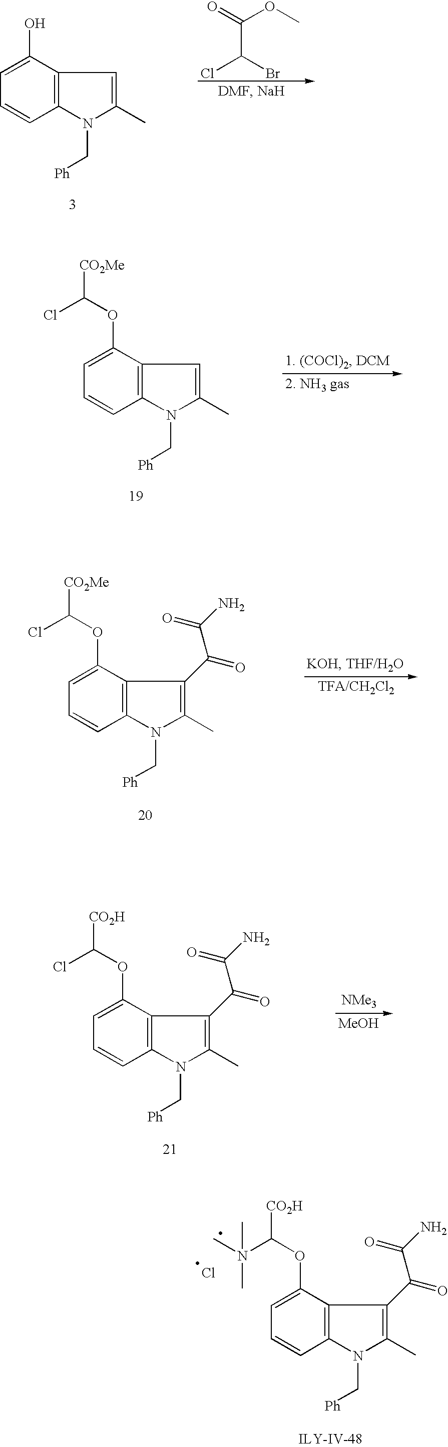

2-(3-(2-amino-2-oxoacetyl)-1-benzyl-2-methyl-1H-indol-4-yloxy)-2-(trimethylamino)acetic acid hydrochloride salt (ILY-IV-48) 1-Benzyl-2-methyl-1H-indol-4-ol 3 (1 mmole) is dissolved in anhydrous dimethylformamide (20 mL). To the solution sodium hydride 60% in mineral oil (1.2 mmole) is added. The mixture is stirred at room temperature for 1 h. To the mixture chloro-bromo-acetic acid methyl ester (1.2 mmole) is added. The mixture is stirred at room temperature for 18 h. The reaction is diluted with ethyl acetate (300 mL) and washed with H₂O (4×100 mL) and brine (1×100 mL). The organic layer is separated, dried with magnesium sulfate and concentrated. The residue is purified by column chromatography to afford 19.

The corresponding acetic acid methyl ester 19 (1 mmole) is dissolved in anhydrous dichloromethane (50 mL). To the solution oxalyl chloride (1.1 mmole) is added. The mixture is left to stir at room temperature for 2 h. NH₃ gas is then bubbled through the solution for 30 minutes. The mixture is left to stir at room temperature for 1 h. The dichloromethane is evaporated and the residue is dissolved in ethyl acetate (200 mL) and washed with H₂O (3×200 mL) and brine (1×300 mL). The organic layer is to be separated, dried with magnesium sulfate and concentrated to afford 20.

Compound 20 (1 mmole) is dissolved in THF:H₂O 4:1 (10 mL). To the mixture 0.5054 N potassium hydroxide solution is added. The mixture is left to stir at room temperature for 18 h. The reaction mixture is evaporated to dryness. The residue is dissolved in H₂O (5 mL) and acidified to pH 4 with 2M HCl. The resulting precipitate is collected by filtration washed with H₂O and dried to afford 21.

Compound 21 (1 mmole) is dissolved in trimethylamine methanol solution (15 mL) in a pressure tube. The mixture is stirred 50° C. for 12 h. The reaction mixture is evaporated to dryness. The residue is triturated with ether and dried to afford ILY-IV-48.

### Example 14.13

### COMPOUND 2-11

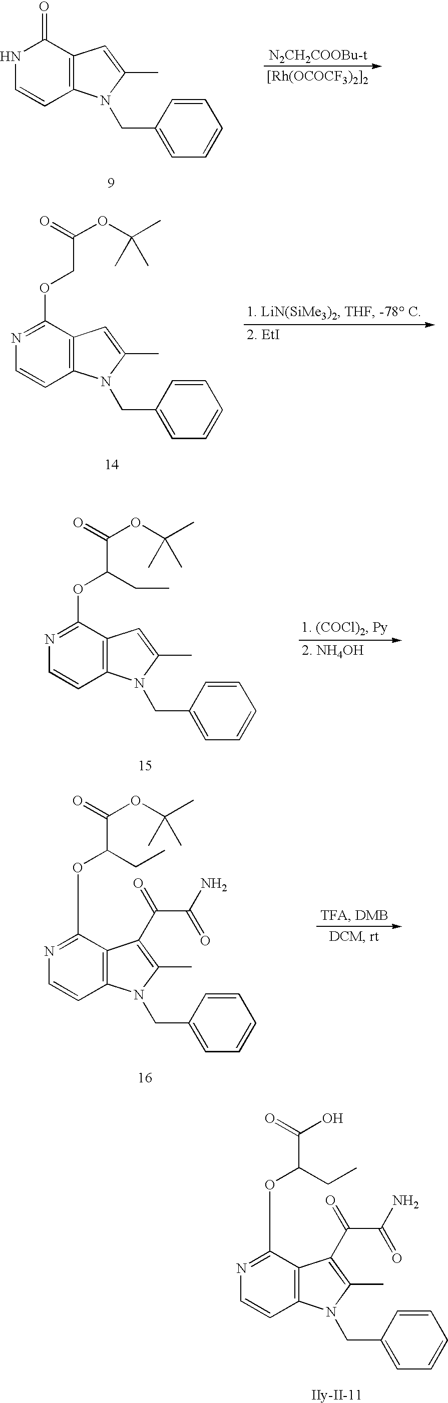

(1-Benzyl-2-methyl-1H-pyrrolo[3,2-c]pyridin-4-yloxy)-acetic acid tert-butyl ester, 14: 1-Benzyl-2-methyl-1,5-dihydro-pyrrolo[3,2-c]pyridin-4-one, 9 (1.0 g, 4.20 mmol) was dissolved in a dry dichloroethane (500 mL). To the mixture Rh₂(OCOCF₃)₄ (132 mg, 0.202 mmol) was added. The reaction mixture was heated to reflux and then to the reaction mixture a solution of tert-butyl diazoacetate (0.65 mL, 4.20 mmol) in dry dichloroethane (50 mL) was added dropwise over 16 h under refluxing. After addition the reaction mixture was stirred for 1 h under refluxing. Then the reaction mixture was cooled to room temperature. The mixture was concentrated and the residue was purified by silica gel chromatography (hexane to hexane:ethyl acetate, 3:1) to afford (1-benzyl-2-methyl-1H-pyrrolo[3,2-c]pyridin-4-yloxy)-acetic acid tert-butyl ester, 14 Yield: 700 mg, (51%)

2-(1-Benzyl-2-methyl-1H-pyrrolo[3,2-c]pyridin-4-yloxy)-butyric acid tert-butyl ester, 15: (1-Benzyl-2-methyl-1H-pyrrolo[3,2-c]pyridin-4-yloxy)-acetic acid tert-butyl ester, 14 (200 mg, 0.568 mmol) was dissolved in a dry tetrahydrofuran (10 mL) and then cooled to −78° C. To the mixture the tetrahydrofuran solution (1.0 M) of LiN(Si(CH₃)₃)₂ (1.70 mL) was added dropwise at −78° C. The reaction mixture was stirred from −78° C. to −5° C. for 1 h and then the tetrahydrofuran solution (5 mL) of iodoethane (0.15 mL, 1.84 mmol) was added dropwise at −50° C. The mixture was stirred for 4 h from −50° C. to room temperature. The mixture was concentrated and the residue was purified by silica gel chromatography (hexane to hexane:ethyl acetate, 4:1) to afford 2-(1-benzyl-2-methyl-1H-pyrrolo[3,2-c]pyridin-4-yloxy)-butyric acid tert-butyl ester, 15 Yield: 50 mg, (23%)

2-(3-Aminooxalyl-1-benzyl-2-methyl-1H-pyrrolo[3,2-c]pyridin-4-yloxy)-butyric acid tert-butyl ester, 16: 2-(1-benzyl-2-methyl-1H-pyrrolo[3,2-c]pyridin-4-yloxy)-butyric acid tert-butyl ester, 15 (134 mg, 0.352 mmol) was dissolved in a dry chloroform (10 mL). To the mixture the solution of oxalyl chloride (0.10 mL, 1.13 mmol) in chloroform (5 mL) was added dropwise at room temperature. Then pyridine (0.05 mL) was added slowly to the mixture at room temperature. After addition the mixture was stirred at room temperature for 18 h. The mixture was poured into icy 20% NH₄OH solution (100 mL) and stirred for 1 h. The mixture was diluted with dichloromethane (20 mL). The organic layer was separated and aqueous layer was extracted with dichloromethane (2×20 mL). The organic layers were combined and dried over anhydrous MgSO₄. The mixture was filtered. The filtrate was concentrated and the residue was purified by silica gel chromatography (hexan to hexane:ethyl acetate, gradient 1:1) to afford 2-(3-aminooxalyl-1-benzyl-2-methyl-1H-pyrrolo[3,2-c]pyridin-4-yloxy)-butyric acid tert-butyl ester, 16 as a yellow solid. Yield: 62 mg, (39%)

2-(3-Aminooxalyl-1-benzyl-2-methyl-1H-pyrrolo[3,2-c]pyridin-4-yloxy)-butyric acid, IIy-II-11: 2-(3-aminooxalyl-1-benzyl-2-methyl-1H-pyrrolo[3,2-c]pyridin-4-yloxy)-butyric acid tert-butyl ester, 16 (26 mg, 0.0576 mmol) was dissolved in dichloromethane (2 mL). To the mixture 1,3-dimethoxybenzene (0.023 mL, 0.172 mmol) was added at room temperature. The mixture was cooled to 0° C. for 30 min. To the mixture trifluoroacetic acid (0.015 mL, 0.234 mmol) was added at 0° C. After addition the mixture was stirred at 0° C. for 1 h. Then mixture was warmed up to room temperature and stirred for 2 h at room temperature. Then more trifluoroacetic acid (0.1 mL) was added and the mixture was stirred at room temperature for 18 h. The mixture was concentrated and H-NMR indicated the reaction was not completed. The residue was redissolved in dichloromethane (5 mL) and then trifluoroacetic acid (0.5 mL) was added at room temperature. The mixture was stirred at room temperature for 6 h. The mixture was concentrated and the residue was purified by silica gel preparative thin layer chromatography (hexane:ethyl acetate, 1:1) to afford 2-(3-aminooxalyl-1-benzyl-2-methyl-1H-pyrrolo[3,2-c]pyridin-4-yloxy)-butyric acid, IIy-II-11 as a light yellow solid. Yield: 11 mg, (48%) ¹H NMR: 05-43-128-2, (400 MHz, DMSO-d6)

δ, 8.09 (br, s, 1H, NH), 7.72 (d, 1H), 7.54 (br, s, 1H, NH), 7.20-7.38 (m, 3H), 7.18 (d, 1H), 7.08 (d, 2H), 5.50 (br, s, 2H, PhCH₂N), 5.02 (t, 1H, CHOAr), 2.41 (br, s, 3H, Me), 1.92 (q, 2H, Et), 1.02 (t, 3H, Et), ppm.

MS (ES): 395.98 [M+1].

### Example 14.14A

### COMPOUND 5-33

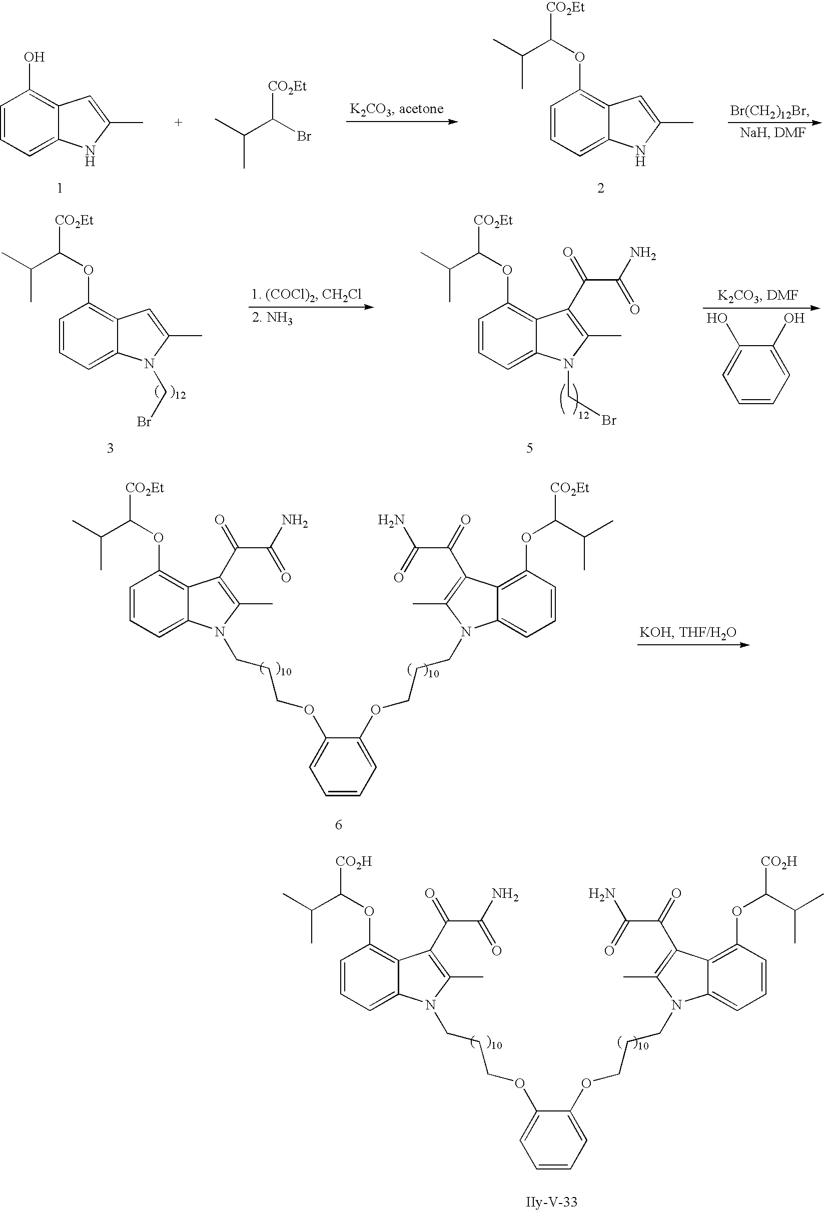

2,2′-(1,1′-(12,12′-(1,2-phenylenebis(oxy))bis(dodecane-12,1-diyl))bis(3-(2-amino-2-oxoacetyl)-2-methyl-1H-indole-4,1-diyl))bis(oxy)bis(3-methylbutanoic acid) (ILY-V-33) Hydroxy indole 1 (1 mmol) and tert-butyl 2-bromo-3-methylbutanoate (1 mmol) is dissolved in 10 mL acetone. To this solution at room temperature is added anhydrous potassium carbonate (2 mmol) and the stirred mixture is refluxed for 12 hours. The solid is removed by filtration and followed by column chromatography to give 2.

Compound 2 (1 mmole) is dissolved in anhydrous dichloromethane (50 mL). To the solution, oxalyl chloride (1.1 mmole) is added. The mixture is left to stir at room temperature for 2 h. NH₃ gas is then bubbled through the solution for 30 minutes. The mixture is left to stir at room temperature for 1 h. The dichloromethane is evaporated and the residue is dissolved in ethyl acetate (200 mL) and washed with H₂O (3×200 mL) and brine (1×300 mL). The organic layer is separated, dried with magnesium sulfate and concentrated to afford 3.

The indole intermediate 3 (1 mmole) in dry DMF (10 mL), at 0° C. under nitrogen, is added with 95% sodium hydride (1.2 mmole). The mixture is stirred at 0° C. for 0.5 h and then added dropwise over 10 minutes to a solution of 1,12-dibromododecane (1.5 mmole) in dry DMF (20 mL) at 0° C. The mixture is stirred at 0° C. for 5 h and at room temperature for 19 h. The reaction 1s cooled to 0° C., quenched with ammonium chloride solution (10 mL), and diluted with dichloromethane (100 mL). The mixture is washed with ammonium chloride solution (50 mL) and the aqueous phase extracted with dichloromethane (4×25 mL). The combined organic phase is washed with brine (100 mL), dried (Na₂SO₄), filtered and evaporated to a red/brown liquid which is further evaporated under high vacuum. The residue is purified by chromatography over silica gel to give 4.

Catechol (1 mmole) is added to sodium hydride (2.2 mmole) in dry DMF (12 mL), at 0° C. under nitrogen. After 0.5 h this mixture is added to the bromide 4 (2.05 mmole) in dry DMF (20 mL), at 0° C. under nitrogen. The reaction is maintained at 0° C. for 8 h and quenched with ammonium chloride solution (15 mL), diluted with dichloromethane (100 mL) and washed with ammonium chloride solution (50 mL). The organic phase is separated and the aqueous phase extracted with dichloromethane (2×25 mL). The combined organic phase is washed with brine (75 mL) dried (Na₂SO₄), filtered and evaporated to a yellow/orange syrup. Purification can be effected by chromatography over silica gel, using chloroform/ethyl acetate as the eluant, give the protected dimer product.

The dimer product (0.9 mmole) and 1,3-dimethoxybenzene (3 mmole) in dry dichloromethane (20 mL), at room temperature under nitrogen, is added with trifluoroacetic acid (10 mL). The solution is stirred for 1 h and the solvents evaporated below 25° C. The residue is triturated with ether (50 mL) and the solid removed by filtration and washed with ether (100 mL). The solid is triturated with ether (50 mL), filtered and washed with ether (50 mL). The product is dried in vacuo to give ILY-V-33.

### Example 14.14B

### COMPOUND 5-33

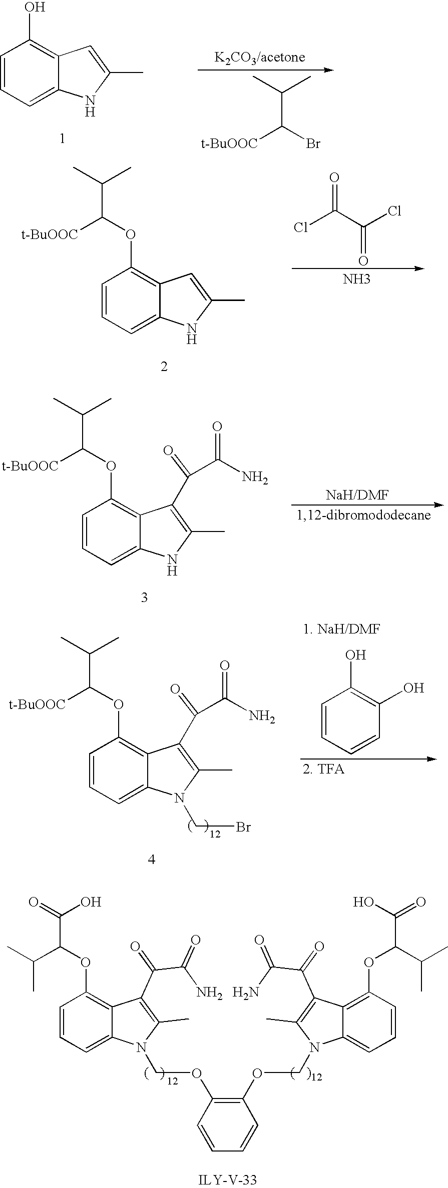

3-Methyl-2-(2-methyl-1H-indol-4-yloxy)-butyric acid ethyl ester (2): A mixture of 4-hydroxy-2-methylindole (1) (1.5 g, 0.010 mole), 2-bromo-3-methyl-butyric acid ethyl ester (2.2 g, 0.010 mole) and potassium carbonate (excess) in acetone (50 mL) was refluxed for 3 days. The reaction mixture was filtered, and the filtrate was concentrated. The residue was purified by column chromatography (20:1 Hex:EtOAc) to afford intermediate 2. Yield: 1.88 g, 71%

2-[1-(12-Bromo-dodecyl)-2-methyl-1H-indol-4-yloxy]-3-methylbutyric acid ethyl ester (3): To a mixture of NaH (60% in mineral oil, 0.42 g, 10 mmole) in anhydrous DMF (20 mL), 3-methyl-2-(2-methyl-1H-indol-4-yloxy)-butyric acid ethyl ester (2) (1.88 g, 7.0 mmole) and dibromododecane (2.30 g, 7.0 mmole) were added. The mixture was stirred at room temperature for 18 h. The reaction was diluted with ethyl acetate (50 mL) and washed with water (3×30 mL). The organic layer was separated, dried over sodium sulphate and concentrated. The residue was purified by column chromatography (10:1 Hex:EtOAc) to afford intermediate (3) Yield: intermediate (3) 1.32 g, 35%, by-product (4) 1.56 g, 31%.

2-[3-Aminooxalyl-1-(12-bromo-dodecyl)-2-methyl-1H-indol-4-yloxy]-3-methyl-butyric acid ethyl ester (5): To a solution of intermediate 3 (0.50 g, 0.959 mmole) in anhydrous dichloromethane (200 mL), oxalyl chloride (0.12 g, 0.95 mmole) was added at 0° C. The mixture was stirred for 1 h. Ammonia gas was bubbled through the reaction mixture for 20 minutes. The mixture was stirred for an addition hour and then concentrated. The residue was diluted with ethyl acetate (30 mL) and washed with water (3×30 mL). The organic layer was separated, dried over sodium sulphate and concentrated to afford intermediate (5) as a yellow solid. Yield: 0.44 g, 77%

2-{3-Aminooxalyl-1-[12-(2-{12-[3-aminooxalyl-4-(1-ethoxycarbonyl-2-methyl-propoxy)-2-methyl-indol-1-yl]-dodecyloxy}-phenoxy)-dodecyl]-2-methyl-1H-indol-4-yloxy}-3-methyl-butyric acid ethyl ester (6): A mixture of intermediate 5 (474 mg, 0.8 mmol), catechol (40 mg, 0.36 mmol) and potassium carbonate (excess) in DMF (5 mL) was stirred at room temperature for 72 h. The reaction was filtered and the filtrate was poured onto crushed ice (20 mL). The mixture was extracted with dichloromethane (3×30 mL). The organic layer was separated, dried over sodium sulphate and concentrated. The residue was purified by column chromatography (1% MeOH in CHCl₃) to afford intermediate (6) and recovered intermediate (5) (205 mg). Yield: 0.060 g, 7%.

2-{3-Aminooxalyl-1-[12-(2-{12-[3-aminooxalyl-4-(1-carboxy-2-methyl-propoxy)-2-methyl-indol-1-yl]-dodecyloxy}-phenoxy)-dodecyl]-2-methyl-1H-indol-4-yloxy}-3-methyl-butyric acid (IIy-V-33): To a solution of intermediate 6 (55 mg, 0.05 mmol) in THF/CH₃OH/H₂O (1:1:1, 2 mL:2 mL:2 mL), potassium hydroxide (0.06 g, 0.11 mmole) was added. The mixture was stirred at room temperature for 4 h. The solution was evaporated and the residue was neutralized with 1M HCl at 0° C. The solid was collected by filtration and washed with water and then hexane to afford IIy-V-33 as a yellow solid. Yield: 0.035 g, 67%. ¹H NMR (400 MHz, DMSO-d₆), δ, ppm: δ 12.51(brs, 2H), 8.10(brs, 2H), 7.62 (brs, 2H), 7.11-7.14(m, 4H), 7.92-7.96 (m, 2H), 7.81-7.84 (m, 2H), 6.42(d, 2H), 4.68(d, 2H), 4.15 (t, 4H), 3.92 (t, 4H), 2.44 (s, 6H), 2.23(m, 2H), 1.62(m, 4H), 1.20-1.43(m, 36H), 1.08(d, 6H), 0.98(d, 6H) ppm. ES-MS: m/z=1079.44(M+1).

### Example 14.15

### COMPOUND 4-55

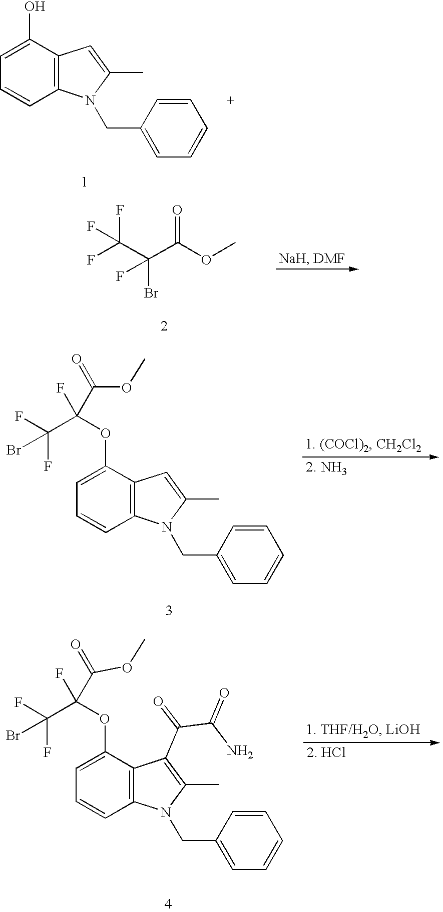

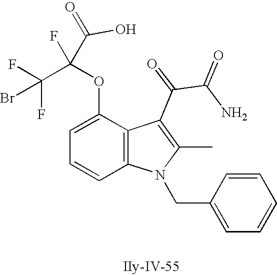

Methyl 2-(1-benzyl-2-methyl-1H-indol-4-yloxy)-3-bromo-2,3,3-trifluoropropanoate (3): To a solution of 1-benzyl-2-methyl-1H-indol-4-ol (1) (0.5 g, 2.1 mmole) in DMF (25 mL), sodium hydride (60% in mineral oil, 0.11 g, 2.75 mmole) was added and the mixture was stirred for 30 minutes at room temperature. Methyl-2-bromo-2,3,3,3-tetrafluoro propionate (0.5 mL, 2.90 mmole) was added to the mixture and stirring was continued at room temperature for 18 h. The reaction was diluted with ethyl acetate (50 mL) and washed with water (3×50 mL) and brine (3×50 mL). The organic layer was separated, dried over magnesium sulphate and concentrated. The residue was purified by preparative TLC (4:1 Hex:EtOAc) to afford intermediate (3) Yield: 0.140 g (17%)

Methyl 2-(3-(2-amino-2-oxoacetyl)-1-benzyl-2-methyl-1H-indol-4-yloxy)-3-bromo-2,3,3-trifluoropropanoate (4): To a solution of the methyl ester (3) (0.14 g, 0.31 mmole) in dichloromethane (60 mL) oxalyl chloride (0.39 g, 0.31 mmole) in dichloromethane (5 mL) was added dropwise at 0 oC. The mixture was stirred for 2 h. Ammonia gas was bubbled through the solution for 30 minutes, and then stirred for an additional 1 h. The reaction solvent was evaporated and the residue was purified by column chromatography intermediate (4) as a solid. 0.122 g, 75%.

2-(3-(2-amino-2-oxoacetyl)-1-benzyl-2-methyl-1H-indol-4-yloxy)-3-bromo-2,3,3-trifluoropropanoic acid (ILY-IV-55): To a solution of the methyl ester (4) (0.95 g, 0.18 mmole) in THF:H2O (4:1, 10 mL), lithium hydroxide mono hydrate (0.01 g, 0.24 mmole) was added. The mixture was stirred at room temperature for 30 minutes. THF was evaporated and the mixture was acidified with 2M HCl to pH 3. The aqueous layer was extracted with ethyl acetate (3×10 mL). The organic layer was separated, dried over magnesium sulphate and concentrated to afford intermediate (ILY-IV-55) as a solid. Yield: (0.09 g, 98%).

### Example 14.16

### COMPOUND 5-44

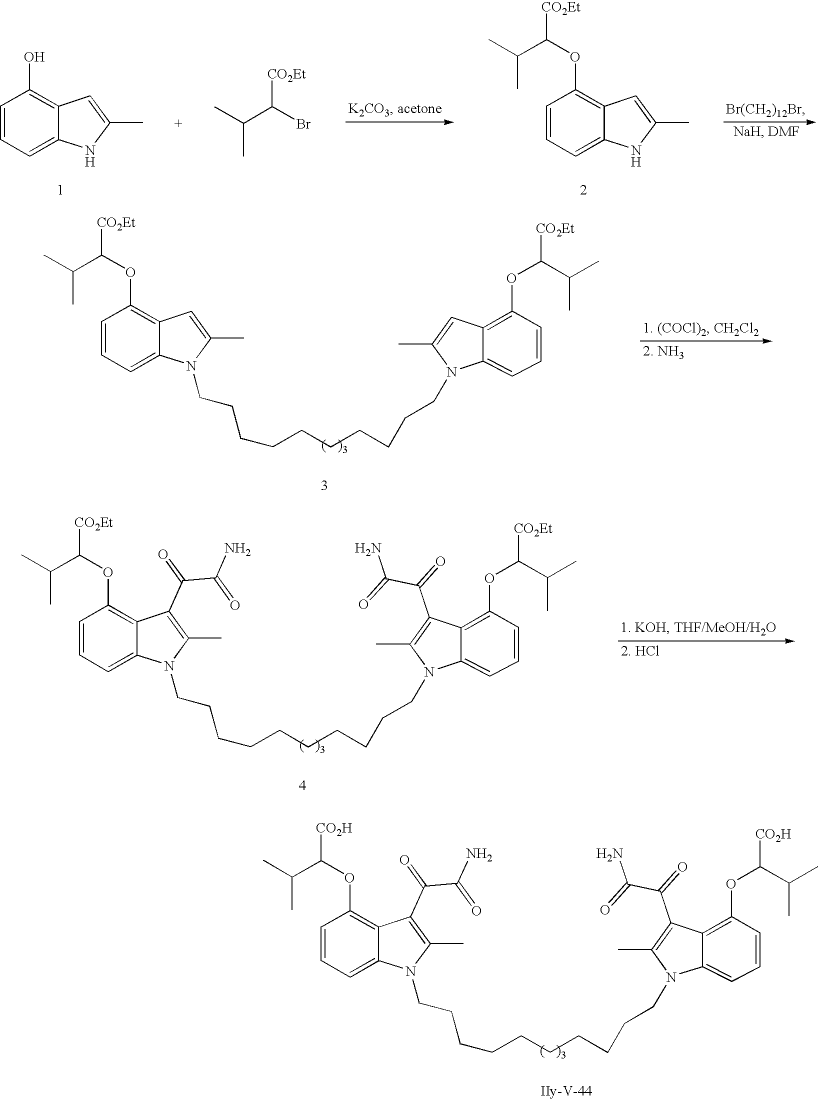

2-(3-Aminooxalyl-1-{12-[3-aminooxalyl-4-(1-ethoxycarbonyl-2-methyl-propoxy)-2-methyl-indol-1-yl]-dodecyl}-2-methyl-1H-indol-4-yloxy)-3-methyl-butyric acid ethyl ester (4): To a solution of intermediate 3 (0.20 g, 0.278 mmole) in anhydrous dichloromethane (20 mL) oxalyl chloride (0.035 g, 0.278 mmole) in anhydrous dichloromethane (20 mL) was added dropwise at 0° C. The mixture was stirred for 1 h. Ammonia was bubbled through the mixture for 20 minutes and stirred for 1 h. The reaction mixture was evaporated. The residue was purified by column chromatography (10:1 CHCl₃:MeOH) to afford intermediate (4) as a yellow solid. Yield: 0.212 g, 91%

2-(3-Aminooxalyl-1-{12-[3-aminooxalyl-4-(1-carboxy-2-methyl-propoxy)-2-methyl-indol-1-yl]-dodecyl}-2-methyl-1H-indol-4-yloxy)-3-methyl-butyric acid (IIy-V-44): A solution of intermediate 4 (100 mg, 0.12 mmol) in THF/CH₃OH/H₂O (1:1:1, 3 mL:3 mL:3 mL) was stirred with 2.2 equivalent of KOH for 4 hr at room temperature. The solution was evaporated and resulting residue was neutralized with 5% HCl at 0° C. The resulting solid was collected by filtration and washed with water and then hexane to afford IIy-V-44 as a yellow solid. Yield: 0.067 g, 72%. ¹H NMR (400 MHz, DMSO-d₆) δ, ppm: 12.51(brs, 2H), 8.02 (brs, 2H), 7.61 (brs, 2H), 7.11-7.14(m, 4H), 6.42(d, 2H), 4.42 (d, 2H), 4.16(t, 4H), 2.41 (s,6H), 2.23(m, 2H), 1.62(m, 4H), 1.20-1.32 (m, 16H), 1.07(d, 6H), 0.96(d, 6H) ppm. ES-MS: m/z=803.12(M+1).

### Example 14.17

### COMPOUND 4-40

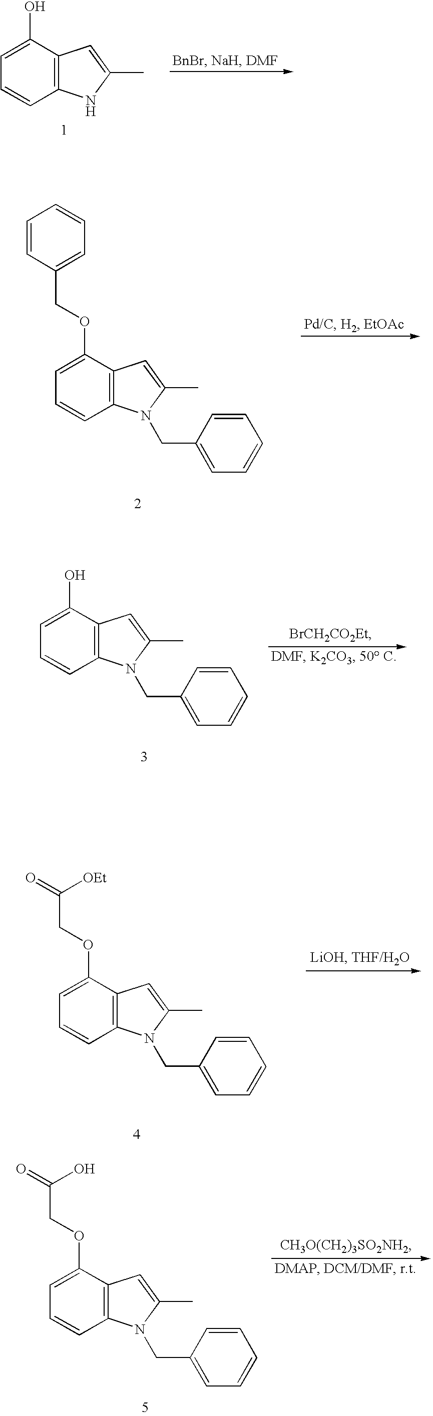

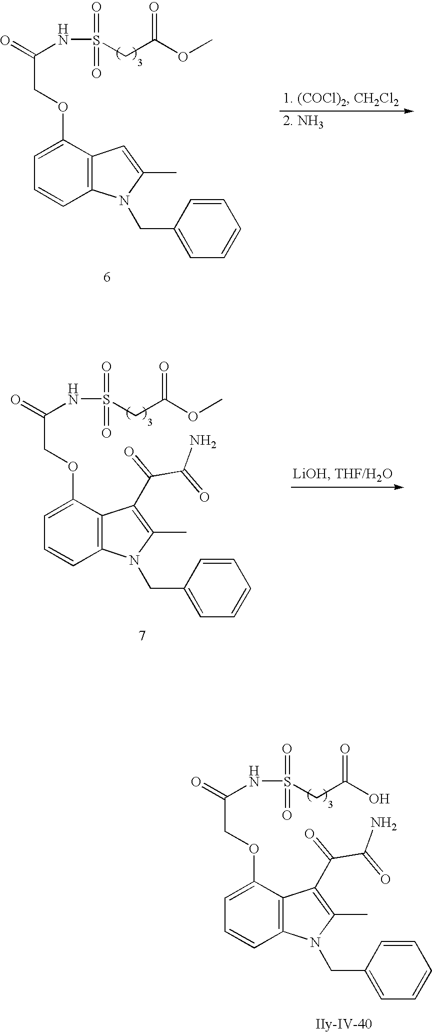

### 4-[2-(3-Aminooxalyl-1-benzyl-2-methyl-1H-indol-4-yloxy)-acetylsulfamoyl]-butyric acid (IIy-IV-40)

1-Benzyl-4-benzyloxy-2-methyl-1H-indole (2): To a suspension of sodium hydride (60% in mineral oil, 27.9 g, 0.69 mole) in anhydrous DMF (500 mL) 4-hydroxyl-2-methyl indole was added and stirred at room temperature for 1 h. A solution of benzyl bromide (82.7 mL, 0.69 mole) in DMF (500 mL) was added dropwise to the mixture. The reaction was stirred at room temperature for 18 h. The reaction mixture was diluted with ethyl acetate (4 L) and washed with water (7×500 mL) and brine (1×500 mL). The organic layer was separated and concentrated. The residue was purified by column chromatography (3:1 Hex:EtOAc) to afford intermediate (2) as an orange oil. Yield: 65 g (58%)

1-Benzyl-2-methyl-1H-indol-4-ol (3): To a solution of 1-Benzyl-4-benzyloxy-2-methyl-1H-indole (2) (35 g, 0.107 mole) in methanol (1 L) and ethyl acetate (500 mL), Pd/C (10%, 17 g) was added. Hydrogen was bubbled through the mixture at room pressure and temperature for 6 h. The reaction mixture was filtered through Celite. The filtrate was concentrated and the residue was purified by column chromatography (6:1 Hex:EtOAc) to afford intermediate (3) as an orange solid. Yield: 22 g (60%)

(1-Benzyl-2-methyl-1H-indol-4-yloxy)-acetic acid ethyl ester (4): To a stirred suspension of K2CO3 (11.7 g, 84.7 mmol), NaI (0.633 g, 4.22 mmol) and 1-benzyl-2-methyl-1H-indol-4-ol (3) (10.0 g, 42.2 mmol) in dry DMF (100 mL) ethyl bromoacetate (5.10 mL, 46.0 mmol) was added dropwise. The reaction mixture was stirred at room temperature for 20 h. The reaction was quenched with water (150 mL) and the mixture was extracted with EtOAc (3×150 mL). The combined organic extracts were washed with water (100 mL), brine (100 mL), dried over Na2SO4 and evaporated. The residue was purified by flash chromatography over silica gel, using 10% EtOAc in hexanes to 25% EtOAc in hexanes) to afford intermediate 4 as a pale yellow solid. Yield: 10.3 g (76%).

(1-Benzyl-2-methyl-1H-indol-4-yloxy)-acetic acid (5): To a solution of (1-benzyl-2-methyl-1H-indol-4-yloxy)-acetic acid ethyl ester (4) (0.80 g, 2.48 mmole) in THF:H2O (4:1, 10 mL), lithium hydroxide monohydrate was added (0.118 g, 4.96 mmole). The mixture was stirred at room temperature for 1 h. THF was evaporated and then crushed ice was added to the aqueous mixture; the resulting solid was collected by filtration to afford intermediate (5) as a solid. Yield: 0.67 g, 92% ¹H NMR: 05-038-055

4-[2-(1-Benzyl-2-methyl-1H-indol-4-yloxy)-acetylsulfamoyl]-butyric acid methyl ester (6): To a solution of (1-benzyl-2-methyl-1H-indol-4-yloxy)-acetic acid (5) (0.189 g, 0.64 mmole) in dichloromethane (15 mL), 4-sulfamoyl-butyric acid methyl ester (0.232 g, 1.28 mmole), EDCI (0.122 g, 0.64 mmole) and DMAP (0.078 g, 0.64 mmole) were added. The mixture was stirred at room temperature for 18 h. The dichloromethane was evaporated to half of the original volume and the mixture was washed with water (2×10 mL). The organic layer was separated and evaporated. The residue was purified by column chromatography (10:1 CHCl3:MeOH) to afford intermediate (6) as a solid. Yield: 0.15 g, 51%

4-[2-(3-Aminooxalyl-1-benzyl-2-methyl-1H-indol-4-yloxy)-acetylsulfamoyl]-butyric acid methyl ester (7): To a solution of 4-[2-(1-benzyl-2-methyl-1H-indol-4-yloxy)-acetylsulfamoyl]-butyric acid methyl ester (6) (0.15 g, 0.32 mmole) in dichloromethane (60 mL) oxalyl chloride (0.41 g, 0.32 mmole) in dichloromethane (5 mL) was added dropwise at 0 oC. The mixture was stirred for 2 h. Ammonia gas was bubbled through the solution for 30 minutes, and then stirred for an additional 1 h. The reaction solvent was evaporated and the residue was purified by column chromatography (2% MeOH in CHCl3) to afford intermediate (7) as a solid. Yield: 0.125 g, 72%.

4-[2-(3-Aminooxalyl-1-benzyl-2-methyl-1H-indol-4-yloxy)-acetylsulfamoyl]-butyric acid (IIy-IV-40): To a solution of intermediate (7) (125 mg, 0.24 mmol) in THF/H2O (4:1, 10 mL) lithium hydroxide monohydrate (0.012 g, 0.528 mmole) was added. The mixture was stirred at room temperature for 30 minutes. THF was evaporated and the resulting residue was neutralized with 5% HCl at 0oC. The green solid was collected by filtration and washed with water (2×20 mL) and hexane (2×20 mL). The colour impurity was removed by dissolving the residue in methanol and stirring with charcoal for 30 minutes. The mixture was filtered through Celite and the filtrate was concentrated to afford IIy-IV-40 as a light yellow solid. Yield: 0.065 g, 53% yield. ¹H NMR (400 MHz, DMSO-d6) δ, ppm: 12.21(brs, 1H), 11.45(brs, 1H), 7.98 (brs, 1H), 7.61 (brs, 1H), 7.23-7.35 (m, 4H), 7.03-7.18 (m, 3H), 6.46 (d, 1H), 5.45 (s, 2H), 4.62(s, 2H), 3.40(t, 2H), 2.54(s, 3H), 2.32(t, 2H), 1.68 (t, 2H). ES-MS: m/z=515.98 (M+1).

Certain such C4-acidic indole and indole related compounds were evaluated for phospholipase activity using the protocol of Example 12. The results are shown in Table 6.

TABLE 6
Inhibition of pancreas secreted human, mouse and porcine PLA₂

| | | | | | | ILYPSA % | | |
|---|---|---|---|---|---|---|---|---|
| | | | ILYPSA IC50 (μM) | | | inhibition at 15 μM | | |
| | Compound | | | | mps | hps | pps | mps |
| structure | ID | MW | hps PLA₂ | pps PLA₂ | PLA₂ | PLA₂ | PLA₂ | PLA₂ |
| 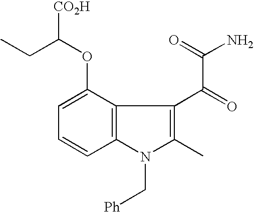 | ILY-IV-20 (4-20) | 394.42 | 0.18 | <0.02 | <0.02 | | | |
| 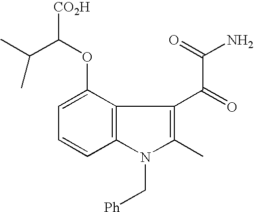 | ILY-IV-22 (4-22) | 408.45 | 0.07 | <0.02 | <0.02 | | | |
| 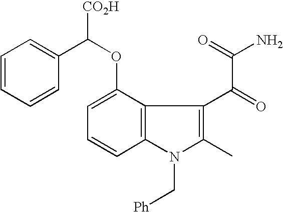 | ILY-IV-32 (4-32) | 442.48 | | | | 41.73 | 38.5 | 47.49 |
| 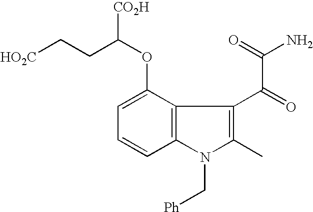 | ILY-IV-33 (4-33) | 438.43 | | 3.76 | | 35.91 | | 50.34 |
| 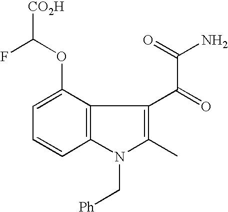 | ILY-IV-24 (4-24) | 384.36 | | 1.42 | | 52.36 | | 63.66 |
| 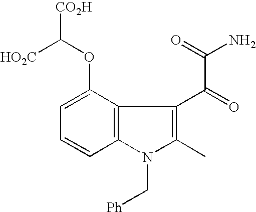 | ILY-IV-8 (4-8) | 410.38 | | 2.25 | | 41 | | 61.22 |
| 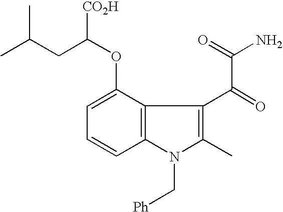 | ILY-IV-47 (4-47) | 422.47 | 2.94 | 0.02 | 2.43 | | | |
| 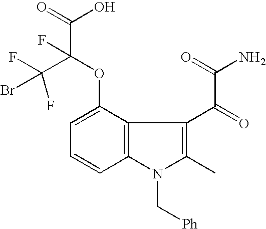 | ILY-IV-55 (4-55) | 513.27 | | | | 33.98 | 74.51 | 42.61 |
| 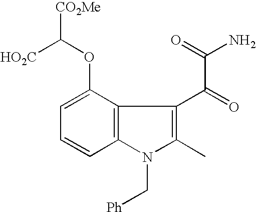 | ILY-IV-59 (4-59) | 424.41 | | | | 10.17 | 56.84 | 35.72 |

### Example 15

### SYNTHESIS OF C4-AMIDE INDOLE AND INDOLE RELATED COMPOUNDS, AND IN-VITRO ASSAY FOR CERTAIN OF SUCH COMPOUNDS FOR THE INHIBITION OF HUMAN, MOUSE AND PORCINE PHOSPHOLIPASE A₂

In this example, various preferred indole and indole-related compounds having specific C4-amide moieties are prepared.

### Example 15.1

### COMPOUND 4-28

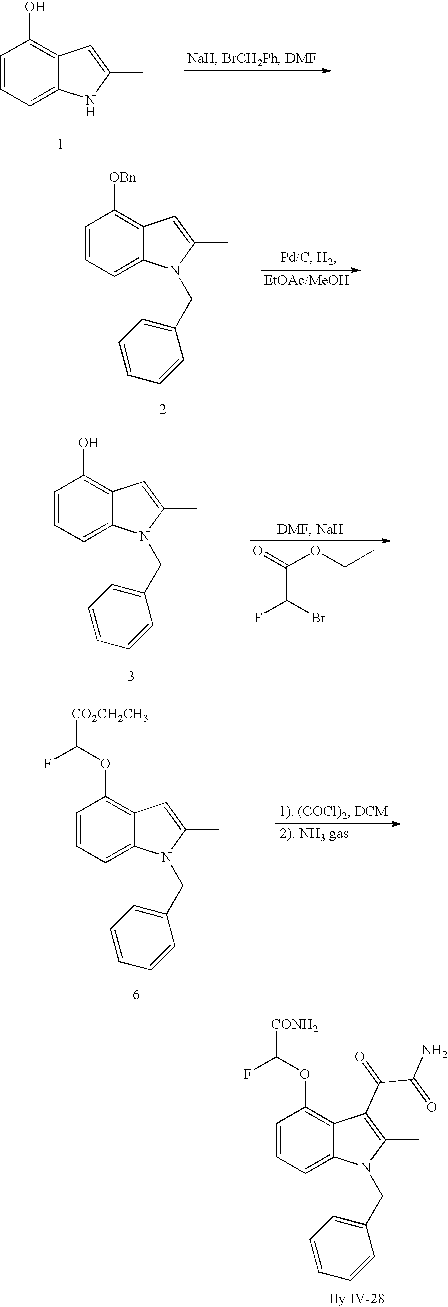

1-Benzyl-4-benzyloxy-2-methyl-1H-indole, 2: 4-hydroxy-2-methyl indole 1 (50 g, 0.339 mole) was dissolved in anhydrous DMF (1 L). To the mixture sodium hydride 60% in mineral oil (27.9 g, 0.697 mole) was added. The mixture was left to stir at rt. for 1 h. To the mixture benzyl bromide (82.7 mL, 0.697 mole) was added drop-wise. The mixture was left to stir at room temperature for 18 h. The reaction was diluted with ethyl acetate (4 L) and washed with water (5×500 mL) then brine (1 L). The organic layer was separated and dried with magnesium sulphate and concentrated. The orange oily residue was purified by column chromatography (6:1 Hexane:EtOAc) to afford 86 g (72%) of 2 as an yellow oil.

1-Benzyl-2-methyl-1H-indol-4-ol 3: 1-Benzyl-4-benzyloxy-2-methyl-1H-indole 2 (86 g, 0.263 mole) was dissolved with ethyl acetate (1.5 L) and methanol (300 mL). To the mixture 10% Pd/C wet (18 g) was added to the solution. The reaction was then subjected to H₂ gas passed through a mercury bubbler at room temperature and 1 atm. The mixture was left to stir for 6 h. The reaction mixture was filtered through Celite and concentrated. The residue was purified by column chromatography (3:1 Hexane:EtOAc) to afford 3 (30 g, 49%) as a cream solid.

(1-Benzyl-2-methyl-1H-indol-4-yloxy)-fluoro-acetic acid ethyl ester 6: 1-Benzyl-2-methyl-1H-indol-4-ol 3 (0.3 g 1.26 mmole) was dissolved in anhydrous dimethylformamide (50 mL). To the solution sodium hydride 60% in mineral oil (66 mg 1.65 mmole) was added. The mixture was stirred at room temperature for 1 h. To the mixture ethyl-2-bromofluoroacetate (0.2 mL, 1.65 mmole) was added. The mixture was stirred at room temperature for 18 h. The reaction was diluted with ethyl acetate (500 mL) and washed with H₂O (5×100 mL) and brine (1×100 mL). The organic layer was separated, dried with magnesium sulfate and concentrated. The residue was purified by column chromatography (6:1 Hexane:EtOAc) to afford 6 (0.14 g, 32%) as an yellow oil.

2-(3-Aminooxalyl-1-benzyl-2-methyl-1H-indol-4-yloxy)-2-fluoro-acetamide IIy-IV-28: To a solution of oxalyl chloride (0.042 mL, 0.478 mmole) was diluted in anhydrous dichloromethane (25 mL). To the solution (1-Benzyl-2-methyl-1H-indol-4-yloxy)-fluoro-acetic acid ethyl ester 6 (0.14 g, 0.398 mmole) in anhydrous dichloromethane (25 mL) was added drop-wise. The mixture was left to stir at room temperature for 2 h. NH₃ gas was then bubbled through the solution for 30 minutes. The mixture was left to stir at room temperature for 1.5 h. The dichloromethane was evaporated and the residue was dissolved in ethyl acetate 300 mL) and washed with H₂O (2×300 mL) and brine (1×300 mL). The organic layer was separated, dried with magnesium sulfate and concentrated. The residue was purified by preparative TLC (3:1 EtOAc:Hex) to afford IIy-IV-28 (0.050 g, 33%).

### Examples 15.2-15.4 and 15.5a

### COMPOUNDS 4-41, 4-42, 4-43 AND 4-45

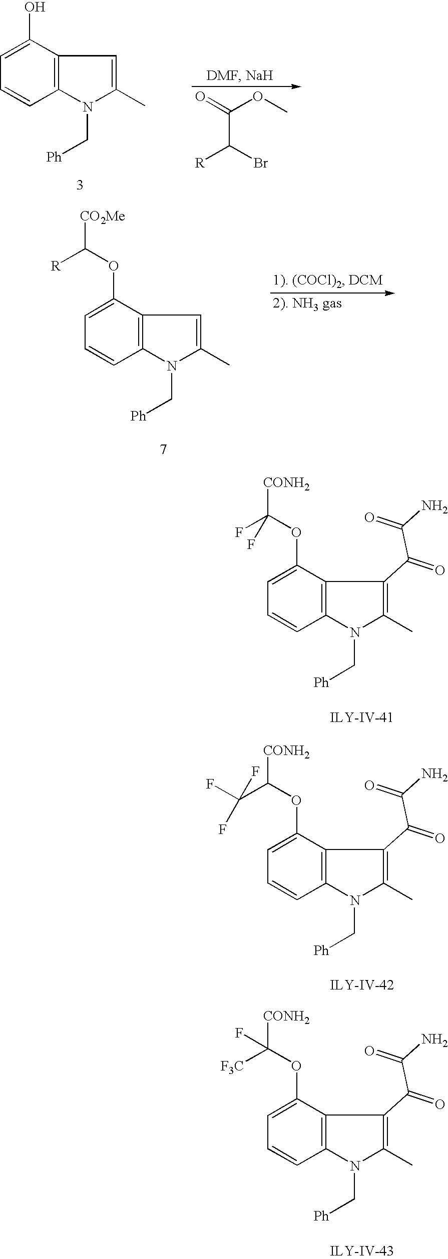

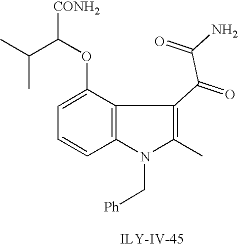

2-(3-(2-amino-2-oxoacetyl)-1-benzyl-2-methyl-1H-indol-4-yloxy)-2,2-difluoroacetamide (ILY-IV-41); 2-(3-(2-amino-2-oxoacetyl)-1-benzyl-2-methyl-1H-indol-4-yloxy)-3,3,3-trifluoropropanamide (ILY-IV-42); 2-(3-(2-amino-2-oxoacetyl)-1-benzyl-2-methyl-1H-indol-4-yloxy)-2,3,3,3-tetrafluoropropanamide (ILY-IV-43); 2-(3-(2-amino-2-oxoacetyl)-1-benzyl-2-methyl-1H-indol-4-yloxy)-3-methylbutanamide (ILY-IV-45)

Alkylation: 1-Benzyl-2-methyl-1H-indol-4-ol 3 (1 mmole) is dissolved in anhydrous dimethylformamide (20 mL). To the solution sodium hydride 60% in mineral oil (1.2 mmole) is added. The mixture is stirred at room temperature for 1 h. To the mixture the corresponding bromo-acetic acid methyl ester (1.2 mmole) is added. The mixture is stirred at room temperature for 18 h. The reaction is diluted with ethyl acetate (300 mL) and washed with H₂O (4×100 mL) and brine (1×100 mL). The organic layer is to be separated, dried with magnesium sulfate and concentrated. The residue is purified by column chromatography to afford 7.

Glyoxamidation and amidation: The corresponding acetic acid methyl ester 7 (1 mmole) is dissolved in anhydrous dichloromethane (50 mL). To the solution oxalyl chloride (1.1 mmole) is added. The mixture is left to stir at room temperature for 2 h. NH₃ gas is then bubbled through the solution for 30 minutes. The mixture is left to stir at room temperature for 5 h. The dichloromethane is evaporated and the residue is dissolved in ethyl acetate (200 mL) and washed with H₂O (3×200 mL) and brine (1×300 mL). The organic layer is separated, dried with magnesium sulfate and concentrated to afford IIy-IV-41, IIy-IV-42, IIy-IV-43, and IIy-IV-45.

### Examples 15.5b

### COMPOUND 4-45

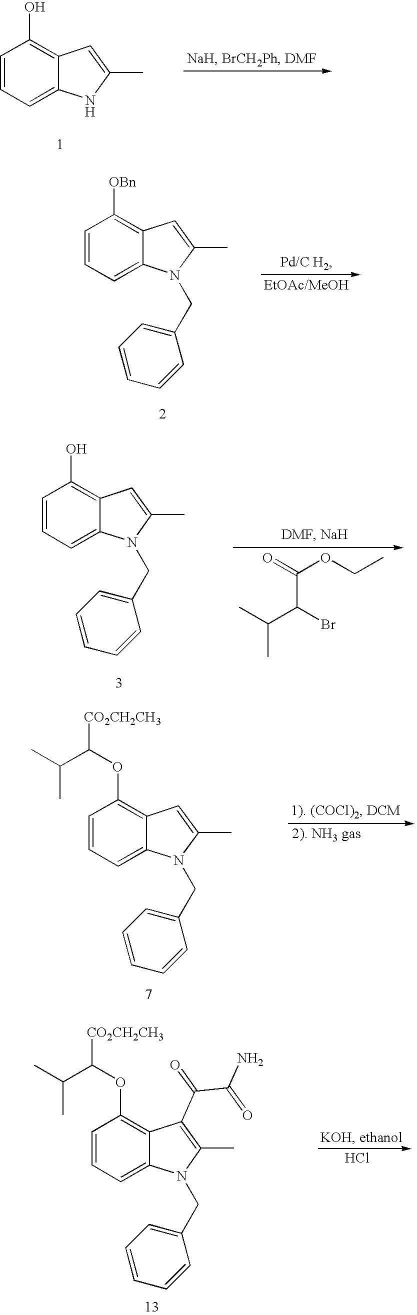

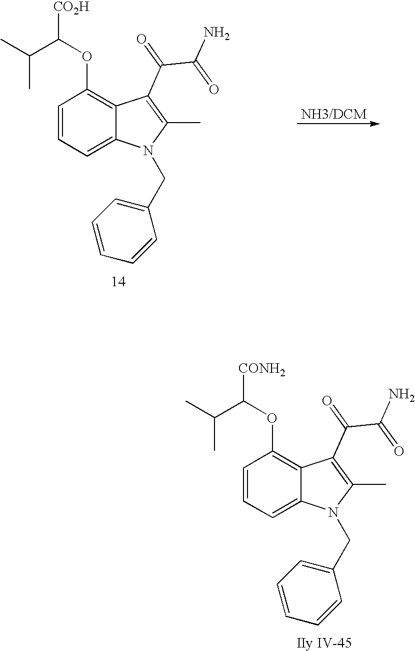

1-Benzyl-4-benzyloxy-2-methyl-1H-indole 2: 4-hydroxy-2-methyl indole 1 (50 g, 0.339 mole) was dissolved in anhydrous DMF (1 L). To the mixture sodium hydride 60% in mineral oil (27.9 g, 0.697 mole) was added. The mixture was left to stir at rt. for 1 h. To the mixture benzyl bromide (82.7 mL, 0.697 mole) was added drop-wise. The mixture was left to stir at room temperature for 18 h. The reaction was diluted with ethyl acetate (4 L) and washed with water (5×500 mL) then brine (1 L). The organic layer was separated and dried with magnesium sulphate and concentrated. The orange oily residue was purified by column chromatography (6:1 Hexane:EtOAc) to afford 86 g (72%) of 2 as an yellow oil.

1-Benzyl-2-methyl-1H-indol-4-ol 3: 1-Benzyl-4-benzyloxy-2-methyl-1H-indole 2 (86 g, 0.263 mole) was dissolved with ethyl acetate (1.5 L) and methanol (300 mL). To the mixture 10% Pd/C wet (18 g) was added to the solution. The reaction was then subjected to H₂ gas passed through a mercury bubbler at room temperature and 1 atm. The mixture was left to stir for 6 h. The reaction mixture was filtered through Celite and concentrated. The residue was purified by column chromatography (3:1 Hexane:EtOAc) to afford 3 (30 g, 49%) as a cream solid.

2-(1-Benzyl-2-methyl-1H-indol-4-yloxy)-3-methyl-butyric acid ethyl ester 7: 1-Benzyl-2-methyl-1H-indol-4-ol 3 (0.3 g 1.26 mmole) was dissolved in anhydrous dimethylformamide (20 mL). To the solution sodium hydride 60% in mineral oil (66 mg 1.65 mmole) was added. The mixture was stirred at room temperature for 1 h. To the mixture ethyl-2-bromoisovalerate (0.344 mL, 1.65 mmole) was added. The mixture was stirred at room temperature for 18 h. The reaction was diluted with ethyl acetate (300 mL) and washed with H₂O (4×100 mL) and brine (1×100 mL). The organic layer was separated, dried with magnesium sulfate and concentrated. The residue was purified by column chromatography (10:1 Hexane:EtOAc) to afford a 1:1 mixture of 7:ethyl-2-bromoisovalerate. Further purification by column chromatography (10:1 Hexane:EtOAc) afforded 7 (0.09 g, 19%) as a yellow oil.

2-(3-Aminooxalyl-1-benzyl-2-methyl-1H-indol-yloxy)-3-methyl-butyric acid ethyl ester 13: 2-(1-Benzyl-2-methyl-1H-indol-4-yloxy)-3-methyl-butyric acid ethyl ester 7 (0.09 g, 0.247 mmole) was dissolved in anhydrous dichloromethane (50 mL). To the solution oxalyl chloride (0.026 mL, 0.296 mmole) was added. The mixture was left to stir at room temperature for 1 h. NH₃ gas was then bubbled through the solution for 30 minutes. The mixture was left to stir at room temperature for 1 h. The dichloromethane was evaporated and the residue was dissolved in ethyl acetate (200 mL) and washed with H₂O (3×200 mL) and brine (1×300 mL). The organic layer was separated, dried with magnesium sulfate and concentrated to afford 13 (0.23 g, >100%) as a yellow solid (contained inorganic salt). The material was used in next step without further purification.

2-(3-Aminooxalyl-1-benzyl-2-methyl-1H-indol-4-yloxy)-3-methyl-butyric acid 14: 2-(3-Aminooxalyl-1-benzyl-2-methyl-1H-indol-yloxy)-3-methyl-butyric acid ethyl ester 13 (0.15 g, 0345 mmole) was dissolved in anhydrous ethanol (10 mL). To the mixture 0.5054 N potassium hydroxide solution (0.4 mL, 0.403 mmole) was added. The mixture was left to stir at room temperature for 72 h. The reaction mixture was evaporated under high vacuum. The residue was dissolved in H₂O (5 mL) and acidified with 2M HCl. The mixture was left to stir for 30 min. The precipitate was collected by filtration washed and with H₂O to afford 14 (0.03 g, 21%) as a yellow solid.

2-(3-(2-amino-2-oxoacetyl)-1-benzyl-2-methyl-1H-indol-4-yloxy)-3-methylbutanamide (ILY-IV-45) 2-(3-Aminooxalyl-1-benzyl-2-methyl-1H-indol-4-yloxy)-3-methyl-butyric acid 14 (0.03 g, 0.074 mmole) was dissolved in anhydrous dichloromethane (20 mL). To the solution NH₃ gas was bubbled through the solution for 30 minutes. The mixture was left to stir at room temperature for 2 h. The dichloromethane was evaporated and the residue was dissolved in ethyl acetate (50 mL) and washed with H₂O (3×50 mL) and brine (1×30 mL). The organic layer was separated, dried with magnesium sulfate and concentrated to afford the crude ILY-IV-45. After flash column chromatography, the pure product was isolated in 0.029 g (99%) as a yellow solid.

### Example 15.6

### COMPOUND 4-49

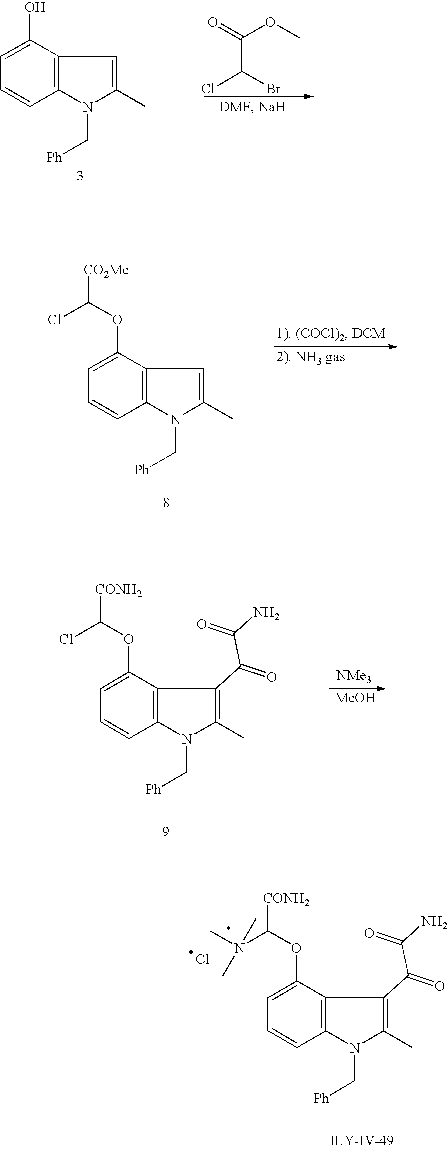

2-(4-(2-amino-1-(trimethylamino)-2-oxoethoxy)-1-benzyl-2-methyl-1H-indol-3-yl)-2-oxoacetamide hydrochloride salt (ILY-IV-49) 1-Benzyl-2-methyl-1H-indol-4-ol 3 (1 mmole) is dissolved in anhydrous dimethylformamide (20 mL). To the solution sodium hydride 60% in mineral oil (1.2 mmole) is added. The mixture is stirred at room temperature for 1 h. To the mixture chloro-bromo-acetic acid methyl ester (1.2 mmole) is added. The mixture is stirred at room temperature for 18 h. The reaction is diluted with ethyl acetate (300 mL) and washed with H₂O (4×100 mL) and brine (1×100 mL). The organic layer is separated, dried with magnesium sulfate and concentrated. The residue is purified by column chromatography to afford 8.

The corresponding acetic acid methyl ester 8 (1 mmole) is dissolved in anhydrous dichloromethane (50 mL). To the solution oxalyl chloride (1.1 mmole) is added. The mixture is left to stir at room temperature for 2 h. NH₃ gas is then bubbled through the solution for 30 minutes. The mixture is left to stir at room temperature for 3 h. The dichloromethane is evaporated and the residue is dissolved in ethyl acetate (200 mL) and washed with H₂O (3×200 mL) and brine (1×300 mL). The organic layer is separated, dried with magnesium sulfate and concentrated to afford 9.

Compound 9 (1 mmole) is dissolved in trimethylamine methanol solution (15 mL) in a pressure tube. The mixture is stirred 50° C. for 12 h. The reaction mixture is evaporated to dryness. The residue is triturated with ether and dried to afford ILY-IV-49.

### Example 15.7

### COMPOUND 4-52

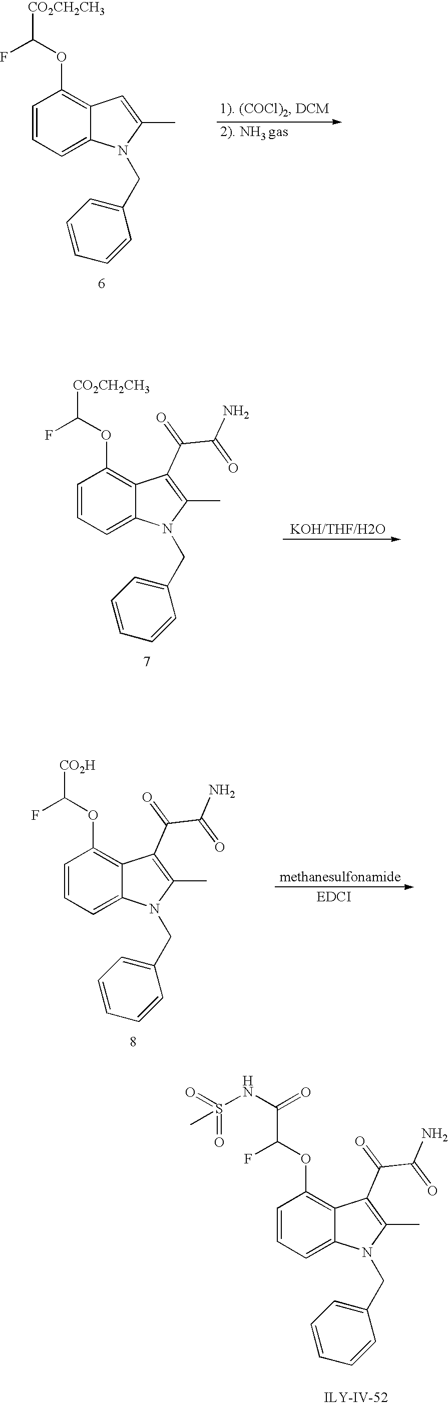

2-(3-(2-amino-2-oxoacetyl)-1-benzyl-2-methyl-1H-indol-4-yloxy)-2-fluoro-N-(methylsulfonyl)acetamide (ILY-IV-52) To a solution of oxalyl chloride (0.478 mmole) is diluted in anhydrous dichloromethane (25 mL). To the solution (1-Benzyl-2-methyl-1H-indol-4-yloxy)-fluoro-acetic acid ethyl ester 6 (0.398 mmole) in anhydrous dichloromethane (25 mL) is added drop-wise. The mixture is left to stir at room temperature for 2 h, and then is cooled to 0° C. NH₃ gas is then bubbled through the solution for 30 minutes. The mixture is left to stir at 0° C. for 2 h. The dichloromethane is evaporated and the residue is dissolved in ethyl acetate 300 mL) and washed with H₂O (2×300 mL) and brine (1×300 mL). The organic layer is to be separated, dried with magnesium sulfate and concentrated. The residue is purified by to afford 7.

Compound 7 (1 mmole) is dissolved in THF:H₂O 4:1 (10 mL). To the mixture 0.5054 N potassium hydroxide solution is added. The mixture is left to stir at room temperature for 18 h. The reaction mixture is evaporated to dryness. The residue is dissolved in H₂O (5 mL) and acidified to pH 4 with 2M HCl. The resulting precipitate is collected by filtration washed with H₂O and dried to afford 8.

To a solution of 2-(3-(2-amino-2-oxoacetyl)-1-benzyl-2-methyl-1H-indol-4-yloxy)-2-fluoroacetic acid 8 (2.3 mmol) in dichloromethane/dimethylformamide mixture (4:1, 10 mL) is added 4-dimethylaminopyridine (3.4 mmol), methanesulfonamide (4.5 mmol) and 1-(3-dimethylaminopropyl)-3-ethylcarbodiimide hydrochloride (2.3 mmol) and the reaction mixture is stirred at room temperature. After 24 h the reaction mixture is diluted with dichloromethane and washed twice with 1N HCl and brine. The organic layer is dried with Na₂SO₄ and evaporated in vacuum. The residue is chromatographed on silica gel to give ILY-IV-52.

### Example 15.8

### COMPOUND 4-53

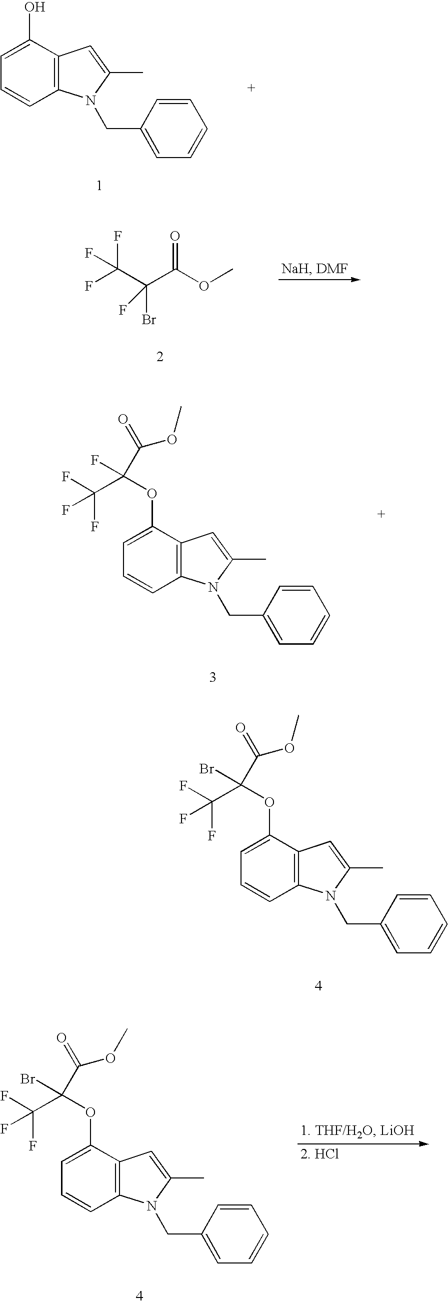

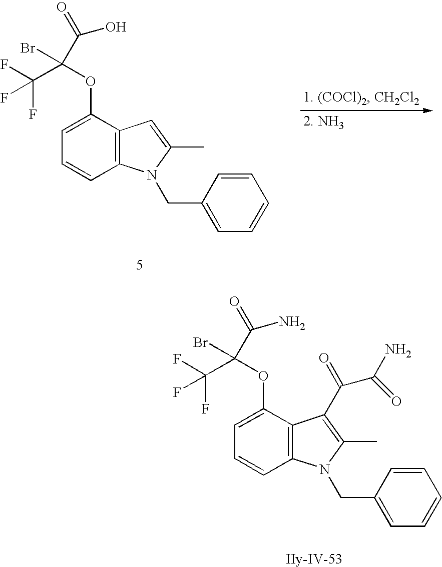

2-(1-Benzyl-2-methyl-1H-indol-4-yloxy)-2-bromo-3,3,3-trifluoro-propionic acid methyl ester (4): To a solution of 1-benzyl-2-methyl-1H-indol-4-ol (1) (0.5 g, 2.1 mmole) in DMF (25 mL), sodium hydride (60% in mineral oil, 0.11 g, 2.75 mmole) was added and the mixture was stirred for 30 minutes at room temperature. Methyl-2-bromo-2,3,3,3-tetrafluoro propionate (0.5 mL, 2.90 mmole) was added to the mixture and stirring was continued at room temperature for 18 h. The reaction was diluted with ethyl acetate (50 mL) and washed with water (3×50 mL) and brine (3×50 mL). The organic layer was separated, dried over magnesium sulphate and concentrated. The residue was purified by preparative TLC (4:1 Hex:EtOAc) to afford intermediate (4) as an orange oil. Intermediate (3) was not the product as expected. Yield: 0.140 g (17%)

2-(1-Benzyl-2-methyl-1H-indol-4-yloxy)-2-bromo-3,3,3-trifluoro-propionic acid (5): To a solution of 2-(1-benzyl-2-methyl-1H-indol-4-yloxy)-2-bromo-3,3,3-trifluoro-propionic acid methyl ester (4) (0.07 g, 0.177 mmole) in THF:H₂O (4:1, 10 mL), lithium hydroxide mono hydrate (0.01 g, 0.238 mmole) was added. The mixture was stirred at room temperature for 30 minutes. THF was evaporated and the mixture was acidified with 2M HCl to pH 3. The aqueous layer was extracted with ethyl acetate (3×10 mL). The organic layer was separated, dried over magnesium sulphate and concentrated to afford intermediate (5) as a pink solid. Yield: (0.066 g, 97%).

2-(3-Aminooxalyl-1-benzyl-2-methyl-1H-indol-4-yloxy)-2-bromo-3,3,3-trifluoro-propionamide (IIy-IV-53): To a solution of 2-(1-benzyl-2-methyl-1H-indol-4-yloxy)-2-bromo-3,3,3-trifluoro-propionic acid (5) (0.066 g, 0.173 mmole) in dichloromethane (20 mL), oxalyl chloride (0.035 mL, 0.381 mmole) was added. The mixture was stirred at room temperature for 1 h. Ammonia was bubbled through the reaction mixture for 30 minutes and stirred for 1 h. at room temperature. The dichloromethane was evaporated. The residue was diluted in ethyl acetate (50 mL) and washed with water (3×50 mL) and brine (3×50 mL). The organic layer was separated, dried over magnesium sulphate and concentrated. The residue was purified by preparative TLC (3:1 EtOAc:Hex) to afford IIy-V-53 as a yellow solid. Yield: 0.02 g (22%), ¹H NMR (400 MHz, DMSO-d₆) δ, ppm: 8.25 (brs, 1H), 8.15 (brs, 1H), 7.90 (brs, 1H), 7.62 (brs, 1H), 7.52 (d, 1H), 7.38-7.18 (m, 4H), 7.10-6.95 (m, 2H), 5.57 (s, 2H), 2.50 (s, 3H). ES-MS: m/z=513.84 (M+1).

Certain such C4-amide indole and indole related compounds were evaluated for phospholipase activity using the protocol of Example 12. The results are shown in Table 7.

TABLE 7
Inhibition of pancreas secreted human, mouse and porcine PLA₂

| | | | | | | ILYPSA % | | |
|---|---|---|---|---|---|---|---|---|
| | | | ILYPSA IC50 (μM) | | | inhibition at 15 μM | | |
| | Compound | | | | mps | | | mps |
| structure | ID | MW | hps PLA₂ | pps PLA₂ | PLA₂ | hps PLA₂ | pps PLA₂ | PLA₂ |
| 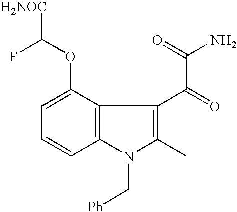 | ILY-IV-28 (4-28) | 383.37 | 2.6 | 0.16 | 1.44 | | | |
| 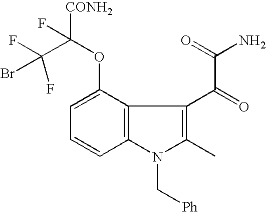 | ILY-IV-53 (4-53) | 512.28 | | 16.01 | | 49 | | 49.68 |
| 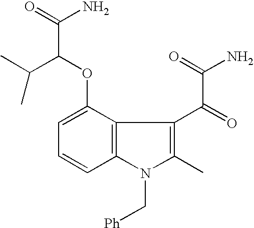 | ILY-IV-45 (4-45) | 407.47 | | 1.03 | | 73.95 | | 65.52 |

### Example 16

### SYNTHESIS OF AZAINDOLE AND AZAINDOLE RELATED COMPOUNDS, AND IN-VITRO ASSAY FOR CERTAIN OF SUCH COMPOUNDS FOR THE INHIBITION OF HUMAN, MOUSE AND PORCINE PHOSPHOLIPASE A₂

In this example, various preferred azaindole and azaindole-related compounds are prepared.

### Example 16.1

### COMPOUND 7-1

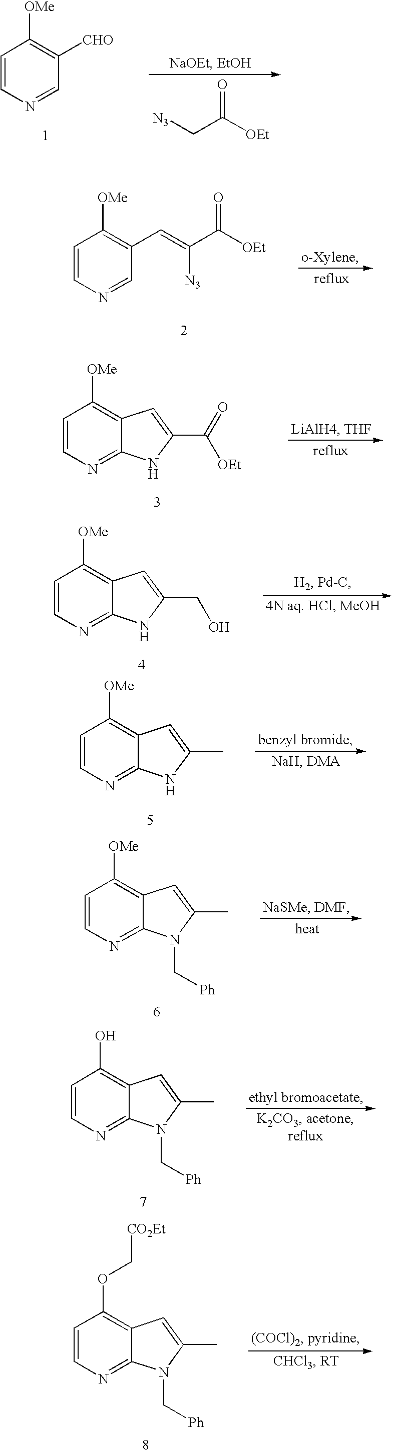

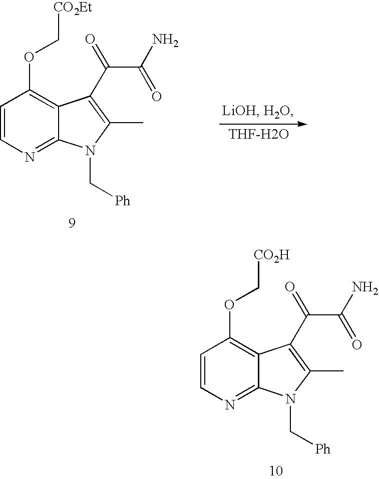

Ethyl α-Azido-β-(4-methoxypyrid-3-yl)-acrylate 2. A homogeneous mixture of 3-formyl-4-methoxypyridine 1 (7.0 g, 54.7 mmol) and ethyl azidoacetate (5.0 g, 36.4 mmol) in anhydrous EtOH (50 mL) was added through a dropping funnel to a well-stirred solution containing Na (0.1.24 g, 54.7 mmol) in anhydrous EtOH (30 mL) under N₂ at −15° C. The mixture was stirred at that temperature for 4 h. During this time the precipitated solid was filtered and washed with ice cooled ethanol (30 mL). The compound was dried under vacuum oven for 3 h to get pure title compound 2 as white crystalline solid. Mp 92-95° C.; Yield: 4.8 g, 53%; ESI MS: m/z 248.9 (M+1).

2-Ethoxycarbonyl-4-methoxypyrrolo-[2,3-b]pyridine 3. A stirred solution of ethyl-α-azido-β-(4-methoxypyrid-3-yl)-acrylate 2 (3.7 g, 14.9 mmol) in dry o-xylene (35 mL) was heated in an oil bath at 170° C. for 25 min. During this time the contents of the flask gained brick red color. After cooling, the mixture was concentrated under high vacuum. The resultant brown residue was purified on silica gel column using 5% methanol in CH₂Cl₂ to give 3 as brick red solid. Mp 195-197° C.; Yield: 3.3 g, 82%; ESI MS: m/z 220.9 (M+1).

(4-Methoxy-1H-pyrrolo[2,3-b]pyridin-2-yl)methanol 4. To a suspension of 2-ethoxycarbonyl-4-methoxypyrrolo-[2,3-b]pyridine 3 (1.90 g, 8.62 mmol) in anhydrous THF (25 mL) was added LiAlH₄ (0.218 g, 17.2 mmol) in small portions under N₂ atmosphere. The mixture was stirred at reflux temperature for 50 min. After cooling, it was poured into cool H₂O (20 mL) and extracted with EtOAc (4×15 mL). The combined organic layers were washed with brine (20 mL) and dried (Na₂SO₄). After filtration, the filtrate was concentrated to dryness and the residue was chromatographed on a silica gel column using 5% methanol in CH₂Cl₂ to give 4 as white solid. Mp 210-212° C.; Yield: 1.10 g, 71%; ESI MS: m/z 178.9 (M+1).

4-Methoxy-2-methyl-1H-pyrrolo[2,3-b]pyridine 5. A suspension of (4-methoxy-1H-pyrrolo[2,3-b]pyridin-2-yl)methanol 4 (0.90 g, 5.05 mmol) and Pd(OH)₂ (100 mg) in methanol containing 4N aq. HCl solution (10 mL) was hydrogenated under hydrogen pressure (50 psi) for 36 h. The acidic mixture was quenched with 1N NaOH solution. Filtration through celite, concentration and purification on silica gel column using 5% methanol in CH₂Cl₂ to gave 5 as pale yellow syrup. Yield: 0.68 g, 83%; ESI MS: m/z 163.01 (M+1).

1-Benzyl-4-methoxy-2-methyl-1H-pyrrolo[2,3-b]pyridine 6. To a suspension of sodium hydride (0.292 g, 9.24 mmol) in dry N,N-dimethyl acetamide (10 mL) was added drop-wise under N₂, a solution of 4-methoxy-2-methyl-1H-pyrrolo[2,3-b]pyridine 5 (0.60 g, 3.70 mmol) in the same solvent (5 mL). The mixture was stirred at room temperature for 45 min. After this time, the solution was cooled in an ice bath, and benzyl bromide (1.25 g, 7.30 mmol) was slowly added. The solution was allowed to warm at room temperature and stirred for 12 h. Then, it was poured into ice water (30 mL) and stirred for 30 min, and the precipitated solid was extracted with ethylacetate (3×20 mL). The organic layer was washed with water and brine. Concentration and purification on silica gel column using 20% ethylacetate in hexanes gave pure title compound 6 as a white solid. Yield: 0.70 g, 68%; mp 129-131° C.; ESI MS: m/z 253.0 (M+1).

1-Benzyl-2-methyl-1H-pyrrolo[2,3-b]pyridin-4-ol 7. To a solution of compound 1-benzyl-4-methoxy-2-methyl-1H-pyrrolo[2,3-b]pyridine 6 (0.45 g, 1.78 mmol) in anhydrous DMF (10 mL) was added NaSMe (0.37 g, 5.35 mmol) under N₂. The reaction mixture was stirred at 80° C. for 45 min. After cooling, the mixture was poured into a saturated solution of NH₄Cl (20 mL), and 1 N HCl (3-4 mL) was added until pH 4-5. The resultant mixture was extracted with EtOAc (5×30 mL), the combined organic extracts were washed with H₂O (2×10 mL) and dried (Na₂SO₄). The solvent was removed under reduced pressure, and the residue was chromatographed on a silica gel column using 5% methanol in CH₂Cl₂ as eluent to give 7 as an amorphous white solid. Yield: 0.30 g, 70%; ESI MS: m/z 238.9 (M+1).

Ethyl 2-(1-benzyl-2-methyl-1H-pyrrolo[2,3-b]pyridin-4-yloxy)acetate 8. A mixture of 1-benzyl-2-methyl-1H-pyrrolo[2,3-b]pyridin-4-ol 7 (0.30 g, 1.26 mmol), 2-bromoethylacetate (1.05 g, 6.29 mmol) and K₂CO₃ (2.0 g) in anhydrous acetone (15 mL) were heated at reflux for 6 h under N₂. After cooling, the mixture was filtered through celite and the filtrate was concentrated to yield a syrup. It was then re-dissolved in ethyl acetate and washed with water (10×2 mL), brine and dried (Na₂SO₄). The solvent was removed under reduced pressure, and the residue was chromatographed on a silica gel column eluting with 40% ethylacetate in hexanes afforded the title compound 8 as an amorphous white solid. Yield: 0.25 g, 61%; ESI MS: m/z 325.0 (M+1).

2-(1-Benzyl-4-yloxyacetic acid ethyl ester-2-methyl-1H-pyrrolo[2,3-b]pyridin-3-yl)-2-oxoacetamide 9. To an ice-cooled solution of ethyl 2-(1-benzyl-2-methyl-1H-pyrrolo[2,3-b]pyridin-4-yloxy)acetate 8 (0.10 g, 0.31 mmol) in anhydrous CHCl₃ (5 mL), oxalyl chloride (0.05 mL, 0.61 mmol) followed by anhydrous pyridine (0.04 mL, 0.60 mmol) was added. The mixture was allowed to attain room temperature and further stirred for 5 h. The mixture was concentrated under vacuum to remove excess unreacted oxalyl chloride. The resultant syrup was and resuspended in CHCl₃ (20 mL) and ammonia gas was passed by cooling to 0° C. for 15 min. The organic layer was washed with water (10×2 mL), dried (Na₂SO₄). The solvent was removed under reduced pressure, and the residue was chromatographed on a silica gel column eluting with 2% ethanol in CH₂Cl₂ to get the title compound 9 as a white solid. Yield: 0.065 g, 53%; mp 139-141° C.; ESI MS: m/z 395.9 (M+1).

2-(1-Benzyl-4-yloxyacetic acid-2-methyl-1H-pyrrolo[2,3-b]pyridin-3-yl)-2-oxoacetamide 10 (IIy-VII-1). To a suspension of 2-(1-benzyl-4-yloxyacetic acid ethyl ester-2-methyl-1H-pyrrolo[2,3-b]pyridin-3-yl)-2-oxoacetamide 9 (0.035 g, 0.08 mmol) in THF—H₂O (1:1, 3 mL), a solution of LiOH.H₂O (0.005 g, 0.13 mmol) was added and the mixture was stirred for 6 h at room temperature. During this time the contents were homogeneous. The pH of the basic solution was set to 4-5 using 1N HCl solution (0.5 mL). The pale yellow solid separated was filtered and washed with H₂O (1 mL) and dried in vacuum oven at 50° C. overnight to get the title compound 10 as a pale yellow solid in high purity. Yield: 0.026 g, 79%; ESI MS: m/z 367.9 (M+1); HPLC: 91.7% purity; ¹H NMR (DMSO-d₆): (5-37-75) δ 8.20 (d, 1H), 7.92 (s, 1H), 7.43 (s, 1H), 7.32-7.22 (m, 3H), 7.18-7.10 (m, 2H), 6.70 (d, 1H), 5.58 (s, 2H), 4.76 (s, 2H), 2.45 (s, 3H) ppm.

### Example 16.2

### COMPOUND 2-1

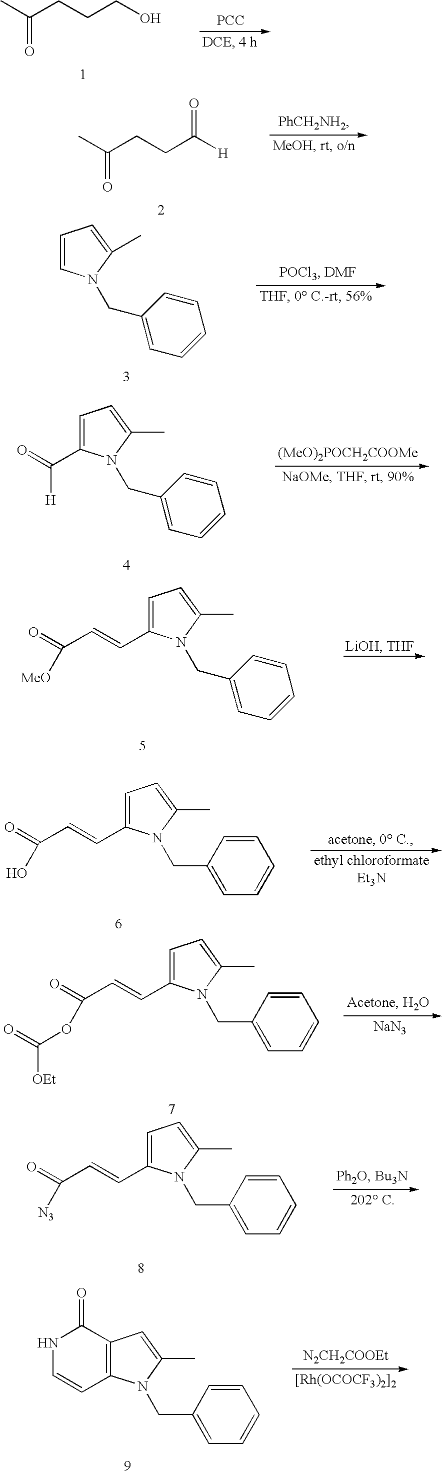

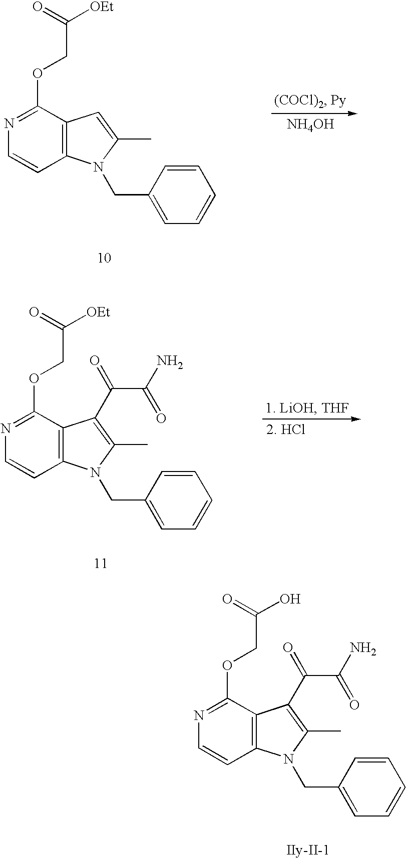

4-Oxo-pentanal, 2: To a stirred suspension of pyridinium chlorochromate (538 g, 2.49 mol) in dichloromethane (4000 mL) at room temperature was added dropwise 3-acetyl-1-propanol (200 g, 1.96 mol) over 5 h. The formed dark mixture was stirred for 1 h at room temperature and then filtered through a pad of silica gel. The silica gel pad was washed with dichloromethane till no product left. The dichloromethane solution was concentrated to afford the crude product as a green liquid. The crude product was purified by distillation under vacuum to afford 4-oxo-pentanal, 2 as clear colorless oil. Yield: 94.6 g (51%).

1-Benzyl-2-methyl-1H-pyrrole, 3: To a stirred mixture of 4-oxo-pentanal (94.6 g, 0.945 mol) in dry methanol (400 mL) and molecular sieve (4A, 100 g) at room temperature was added dropwise benzylamine solution (125 mL, 1.13 mol) in dry methanol (125 mL). The formed dark solution was stirred for 18 h at room temperature and then the reaction mixture was filtered and concentrated. The crude product was purified by silica gel chromatography (hexane to hexane:ethyl acetate, 3:1) to afford 1-benzyl-2-methyl-1H-pyrrole, 3 as a light yellow oil. Yield: 94 g (58%).

1-Benzyl-5-methyl-1H-pyrrole-2-carbaldehyde, 4: POCl₃ (23.46 mL, 246 mmol) was added dropwise to a stirred N,N-dimethylformamide (204 mL) at 0° C. After addition the mixture was stirred for additional 90 minutes. To the mixture was added dropwise the solution of 1-benzyl-2-methyl-1H-pyrrole, 3 (2.71 g, 45 mmol) in tetrahydrofuran (1150 mL). The reaction mixture was allowed to be stirred for 18 h from 0° C. to room temperature. The mixture was concentrated and redissolved in ethyl acetate (2 L). The mixture was washed with saturated Na₂CO₃ (2×500 mL). The Na₂CO₃ solution was extracted with ethyl acetate (7×1 L). The organic layers were combined and concentrated. The crude product was purified by silica gel chromatography (hexane to hexane:ethyl acetate, 7:1) to afford 1-benzyl-5-methyl-1H-pyrrole-2-carbaldehyde, 4 as a light yellow liquid. Yield: 30.8 g (81%).

3-(1-Benzyl-5-methyl-1H-pyrrolo-2-yl)-acrylic acid methyl ester, 5: Sodium (14.45 g, 628 mmol) was added in portions to a dry methanol (420 mL). To the fresh formed sodium methoxide solution was added dropwise the solution of trimethyl phosphonoacetate (50 mL, 302 mmol) in tetrahydrofuran (105 mL) at room temperature. After addition the mixture was stirred for additional 60 min at room temperature. Then to the reaction mixture was added dropwise the solution of 1-benzyl-5-methyl-1H-pyrrole-2-carbaldehyde, 4 (30.8 g, 154 mmol) in tetrahydrofuran (630 mL) at room temperature. The reaction mixture was stirred for 2 h at room temperature. The mixture was concentrated and redissolved in ethyl acetate (1 L). The mixture was washed with 1 M HCl solution, then saturated NaHCO₃, H₂O. The organic solution were dried over MgSO₄ and then filtered, concentrated to afford the crude product, 3-(1-benzyl-5-methyl-1H-pyrrolo-2-yl)-acrylic acid methyl ester, 5 as a light yellow solid. Yield: 40 g

1-Benzyl-2-methyl-1,5-dihydro-pyrrolo[3,2-c]pyridin-4-one, 6: 3-(1-Benzyl-5-methyl-1H-pyrrolo-2-yl)-acrylic acid methyl ester, 5 (40 g) was dissolved in a mixture of tetrahydrofuran (400 mL) and methanol (400 mL). To the mixture a solution of lithium hydroxide monohydrate (20 g, 476 mmol) in H₂O (200 mL) was added. After addition the reaction mixture was stirred for 18 h at room temperature. The reaction mixture was acidified by 2M HCl to pH=4-5. The mixture was concentrated and redissolved in ethyl acetate (2 L). The mixture was washed with H₂O. The water layer was extracted with ethyl acetate (2×1 L). The organic was combined and concentrated to afford a yellow solid which was washed with dichloromethane to afford the product (22.66 g). The washing dichloromethane solution were concentrated and the residue was purified by silica gel chromatography (hexane to hexane:ethyl acetate, 1:3, followed by neat ethyl acetate) to afford 3-(1-Benzyl-5-methyl-1H-pyrrolo-2-yl)-acrylic acid, 6 as a light yellow solid (5.9 g). Yield: 28.56 g, (77%, 2 steps)

1-Benzyl-2-methyl-1,5-dihydro-pyrrolo[3,2-c]pyridin-4-one, 9: 3-(1-Benzyl-5-methyl-1H-pyrrolo-2-yl)-acrylic acid, 6 (26.72 g, 110.9 mmol) was dissolved in a dry acetone (1050 mL). To the suspension mixture triethylamine (35 mL) was added to form a clear solution. The reaction mixture was cooled to 0° C. and then to the cooled reaction mixture a solution of ethyl chloroformate (30 mL, 304 mmol) in dry acetone (650 mL) was added dropwise over 1 hour. After addition the reaction mixture was stirred for 4 h at 0° C. Then to the reaction mixture was added dropwise the solution of sodium azide (14.52 g, 223 mmol) in H₂O (175 mL) over 30 minutes. The reaction mixture was stirred at 0° C. for 2 h. The reaction mixture was poured into ice-water (1 L). Then the mixture was extracted with dichloromethane (3×1 L). The organic layers were combined and dried over MgSO₄. The mixture was filtered and concentrated to afford a crude 8 as a yellow solid (32 g). To the mixture of diphenyl ether (175 mL) and tributylamine (31 mL) which was preheated to 205° C. was added dropwise the solution of crude 8 in diphenyl ether (250 mL) at 205° C. for 1 hour. After addition the mixture was stirred for another hour at 205° C. The mixture was cooled to room temperature and solid was formed. Diethyl ether (500 mL) was added into the reaction mixture to form more solid. The mixture was filtered and the solid was washed with diethyl ether to afford the product (8.81 g). The filtrate was concentrated and the residue was purified by silica gel chromatography (hexane to hexane:ethyl acetate, gradient 1:1 to 1:3; then methanol in dichloromethane, 1% to 5%) to afford 1-benzyl-2-methyl-1,5-dihydro-pyrrolo[3,2-c]pyridin-4-one, 9 as a yellow solid (4.7 g). Yield: 13.51 g, (51%)

(1-Benzyl-2-methyl-1H-pyrrolo[3,2-c]pyridin-4-yloxy)-acetic acid ethyl ester, 10: 1-Benzyl-2-methyl-1,5-dihydro-pyrrolo[3,2-c]pyridin-4-one, 9 (512 mg, 2.15 mmol) was dissolved in a dry dichloroethane (300 mL). To the mixture Rh₂(OCOCF₃)₄ (64 mg, 0.097 mmol) was added. The reaction mixture was heated to reflux and then to the reaction mixture a solution of ethyl diazoacetate (0.25 mL, 2.15 mmol) in dry dichloroethane (30 mL) was added dropwise over 6 h under refluxing. After addition the reaction mixture was stirred for 1.5 h under refluxing. Then the reaction mixture was cooled to room temperature. The mixture was concentrated and the residue was purified by silica gel chromatography (hexane to hexane:ethyl acetate, 5:1) to afford (1-benzyl-2-methyl-1H-pyrrolo[3,2-c]pyridin-4-yloxy)-acetic acid ethyl ester, 10. Yield: 345 mg, (49%)

(3-Aminooxalyl-1-benzyl-2-methyl-1H-pyrrolo[3,2-c]pyridin-4-yloxy)-acetic acid ethyl ester, 11: (1-Benzyl-2-methyl-1H-pyrrolo[3,2-c]pyridin-4-yloxy)-acetic acid ethyl ester, 10 (370 mg, 1.14 mmol) was dissolved in a dry chloroform (37 mL). To the mixture the solution of oxalyl chloride (0.30 mL, 3.43 mmol) in chloroform (10 mL) was added dropwise at room temperature. Then pyridine (0.133 mL) was added slowly to the mixture at room temperature. After addition the mixture was stirred at room temperature for 18 h. The mixture was concentrated and the residue was purified by silica gel chromatography (hexan to hexane:ethyl acetate, gradient 1:1 to 1:3) to afford (3-aminooxalyl-1-benzyl-2-methyl-1H-pyrrolo[3,2-c]pyridin-4-yloxy)-acetic acid ethyl ester, 11 as a yellow solid. Yield: 280 mg, (62%)

(3-Aminooxalyl-1-benzyl-2-methyl-1H-pyrrolo[3,2-c]pyridin-4-yloxy)-acetic acid, IIy-II-1: (3-Aminooxalyl-1-benzyl-2-methyl-1H-pyrrolo[3,2-c]pyridin-4-yloxy)-acetic acid ethyl ester, 11 (90 mg, 0.227 mmol) was dissolved in methanol (20 mL). To the mixture the solution of KOH (1M, 0.25 mL) was added at room temperature. After addition the mixture was stirred at room temperature for 18 h. Then solution of lithium hydroxide monohydrate (90 mg) in H₂O (5 mL) was added. After another hour stirring the mixture was concentrated and the residue was redissolved in methanol (10 mL) and ethanol (10 mL). The mixture was filtered and the filtrate was acidified by hydrogen chloride in ether (1.0 M) to pH=3-4. Solvent was evaporated and the residue was washed with a mixture of dichloromethane:ether (1:1), then water (5 mL) and ether to afford (3-aminooxalyl-1-benzyl-2-methyl-1H-pyrrolo[3,2-c]pyridin-4-yloxy)-acetic acid, IIy-II-1 as a light yellow solid. Yield: 29 mg, (35%)

¹H NMR: 05-43-67, (400 MHz, DMSO-d6)

δ, 12.96 (br, s, 1H, COOH), 7.97 (br, s, 1H, NH), 7.79 (d, 1H), 7.56 (br, s, 1H, NH), 7.22-7.39 (m, 4H), 7.08-7.12 (m, 2H), 5.57 (br, s, 2H, PhCH₂N), 4.80 (br, s, 2H, CH₂OAr) ppm.

MS (ES): 367.99 [M+1].

### Example 16.3

### COMPOUND 2-7

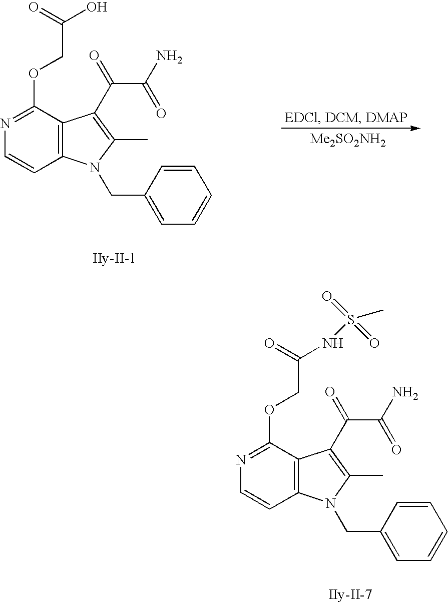

2-[1-Benzyl-4-(2-methanesulfonylamino-2-oxo-ethoxy)-2-methyl-1H-pyrrolo[3,2-c]pyridin-3-yl]-2-oxo-acetamide, IIy-II-7: (3-Aminooxalyl-1-benzyl-2-methyl-1H-pyrrolo[3,2-c]pyridin-4-yloxy)-acetic acid, IIy-II-1 (27 mg, 0.0736 mmol) was suspended in dichloromethane (2 mL). To the mixture 4-dimethylaminopyridine (35 mg, 0.286 mmol) was added at room temperature, followed by methanesulfonamide (30 mg, 0.296 mmol) and N-(3-dimethylaminopropyl)-N″-ethylcarbodiimide hydrochloride (45 mg, 0.234 mmol). After addition the mixture was stirred at room temperature for 24 h. Dichloromethane (20 mL) was added to dilute the reaction mixture. Then reaction mixture solution was washed with 1.0 M HCl, water and dried over MgSO₄. The mixture was filtered. The filtrate was concentrated and the residue was purified by silica gel chromatography (hexane to hexane:ethyl acetate, gradient 1:1 to 1:2; then methanol in dichloromethane, 5% to 15%) to afford 2-[1-benzyl-4-(2-methanesulfonylamino-2-oxo-ethoxy)-2-methyl-1H-pyrrolo[3,2-c]pyridin-3-yl]-2-oxo-acetamide, IIy-II-7 as an off-white solid. Yield: 9 mg, (28

¹H NMR: 05-43-101-2, (400 MHz, DMSO-d6)

δ, 11.62 (br, s, 1H, NHSO₂), 8.16 (br, s, 1H, NH), 7.80 (d, 1H), 7.68 (br, s, 1H, NH), 7.26-7.40 (m, 4H), 7.06-7.12 (m, 2H), 5.58 (br, s, 2H, PhCH₂N), 4.85 (br, s, 2H, CH₂OAr), 3.20 (br, s, 3H, SO₃CH₃) ppm.

MS (EI): 444.85 [M+1], 442.84 [M−1]

### Example 16.4

### COMPOUND 24

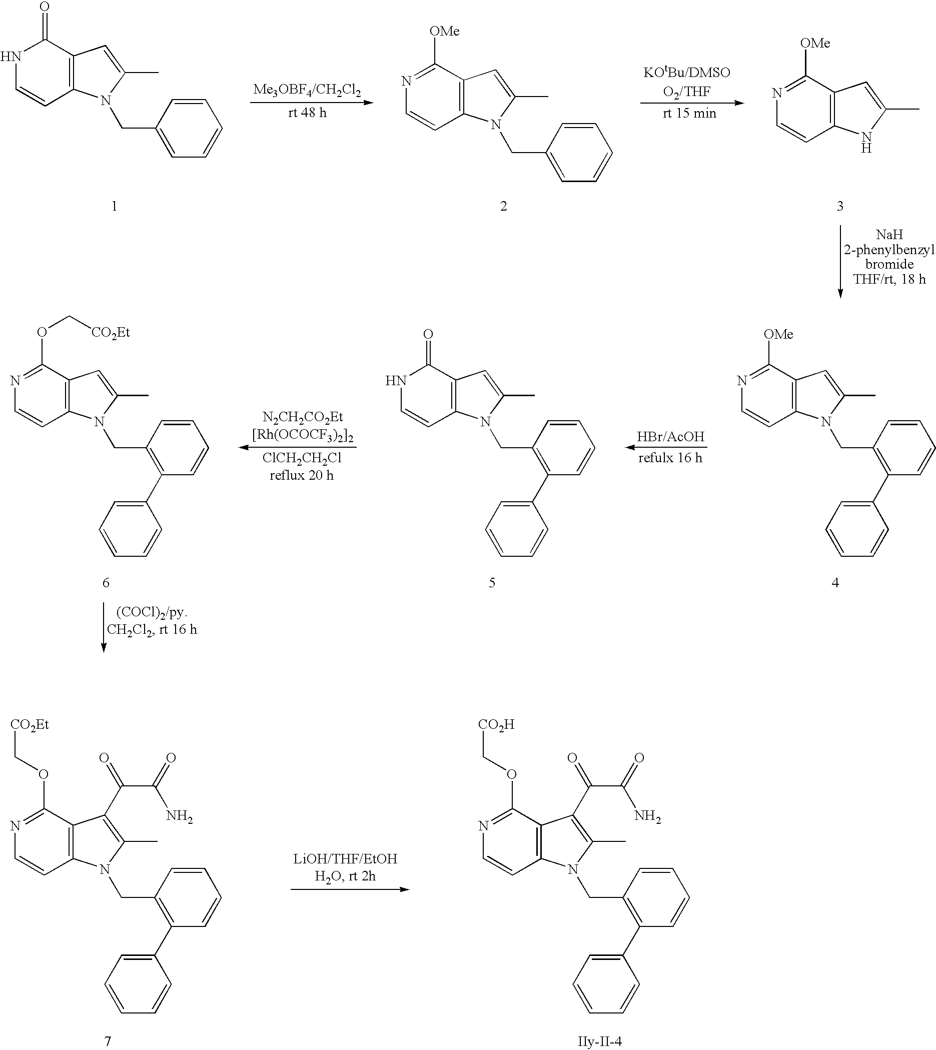

(1-Benzyl-4-methoxy-2-methyl-1H-pyrrolo[3,2-c]pyridine 2. To a stirred suspension of 1-benzyl-2-methyl-1,5-dihydro-pyrrolo[3,2-c]pyridin-4-one 1 (2.0 g, 8.4 mmol) in CH₂Cl₂ (70 mL), Me₃OBF₄ (3.8 g, 25.6 mmol) was added and the reaction mixture was stirred for 48 h, then diluted with CH₂Cl₂ (70 mL). The mixture was washed with water (100 mL), brine (100 mL), dried over Na₂SO₄ and evaporated. Flash chromatography of the residue over silica gel, using 10% EtOAc in hexanes to 25% EtOAc in hexanes) gave product 2 as a pale yellow solid. Yield: 1.6 g (75%).

4-Methoxy-2-methyl-1H-pyrrolo[3,2-c]pyridine 3. To a stirred solution of (1-benzyl-4-methoxy-2-methyl-1H-pyrrolo[3,2-c]pyridine 2 (0.887 mg, 3.52 mmol) in THF (10 mL), DMSO (2.5 mL), followed by KO^{t}Bu (25 mL, 1.0 M in THF) was added dropwise, and then the reaction mixture was treated with O₂ for 15 min at room temperature, quenched with saturated NH₄Cl (20 mL), extracted with EtOAc (3×60 mL). The combined organic extracts were washed with water (50 mL), brine (50 mL), dried over Na₂SO₄ and evaporated. Flash chromatography of the residue over silica gel, using 20% EtOAc in hexanes to 40% EtOAc in hexanes) gave product 3 as a yellow solid. Yield: 560 mg (98%).

1-Biphenyl-2-ylmethyl-4-methoxy-2-methyl-1H-pyrrolo[3,2-c]pyridine 4. To a stirred suspension of NaH (98 mg, 2.5 mmol, 60% in mineral oil) in THF (10 mL), 4-methoxy-2-methyl-1H-pyrrolo[3,2-c]pyridine 3 (280 mg, 1.72 mmol) in THF (3 mL) was added. The mixture was stirred at room temperature for 30 min, and then 2-phenylbenzyl bromide (0.40 mL, 2.2 mmol) was added, stirring was continued for 18 h. The reaction mixture was quenched with saturated NH₄Cl (20 mL), extracted with EtOAc (3×40 mL). The combined organic extracts were washed with water (40 mL), brine (40 mL), dried over Na₂SO₄ and evaporated. Flash chromatography of the residue over silica gel, using 10% EtOAc in hexanes to 25% EtOAc in hexanes) gave product 4 as a yellow foam. Yield: 375 mg (66%).

1-Biphenyl-2-ylmethyl-2-methyl-1,5-dihydro-pyrrolo[3,2-c]pyridin-4-one 5. To a stirred solution of 1-biphenyl-2-ylmethyl-4-methoxy-2-methyl-1H-pyrrolo[3,2-c]pyridine 4 (370 mg, 1.13 mmol) in AcOH (15 mL), 48% of HBr (5 mL) was added. The reaction mixture was heated to 105° C., and then stirred for 16 h, cooled to room temperature and evaporated. The obtained residue was dissolved in CH₂Cl₂ (100 mL), washed with saturated NaHCO₃ (30 mL), brine (30 mL), dried over Na₂SO₄ and evaporated to afford crude product 5, which was used without further purification for next step. Yield: 355 mg (100%).

(1-Biphenyl-2-ylmethyl-2-methyl-1H-pyrrolo[3,2-c]pyridin-4-yloxy)-acetic acid ethyl ester 6. To a stirred solution of 1-biphenyl-2-ylmethyl-2-methyl-1,5-dihydro-pyrrolo[3,2-c]pyridin-4-one 5 (0.355 g, 1.13 mmol) in ClCH₂CH₂Cl (40 mL), [Rh(OCOCF₃)₂]₂ (48 mg, 0.073 mmol) was added, and then a solution of N₂CH₂CO₂Et (0.13 mL. 1.3 mmol) in ClCH₂CH₂Cl (8 mL) was added over 16 h via a syringe pump. The reaction mixture was cooled to room temperature and evaporated. Flash chromatography of the residue over silica gel, using 10% EtOAc in hexanes to 25% EtOAc in hexanes) gave product 6 as a yellow solid. Yield: 105 mg (22%).

(3-Aminooxalyl-1-biphenyl-2-ylmethyl-2-methyl-1H-pyrrolo[3,2-c]pyridin-4-yloxy)-acetic acid ethyl ester 7. To a stirred solution of (1-biphenyl-2-ylmethyl-2-methyl-1H-pyrrolo[3,2-c]pyridin-4-yloxy)-acetic acid ethyl ester 6 (100 mg, 0.250 mmol) in CH₂Cl₂ (10 mL), (COCl)₂ (80 μL, 0.91 mmol), followed by pyridine (40 μL) was added dropwise, and then the reaction mixture was stirred at room temperature for 16 h, treated with NH₃ (g) for 30 min and stirred for another 1 h. The obtained mixture was diluted with EtOAc (40 mL), washed with water (20 mL), brine (20 mL), dried over Na₂SO₄ and evaporated. Flash chromatography of the residue over silica gel, using 50% hexanes in EtOAc to 25% hexanes in EtOAc) gave product 7 as a yellow solid. Yield: 30 mg (25%).

(3-Aminooxalyl-1-biphenyl-2-ylmethyl-2-methyl-1H-pyrrolo[3,2-c]pyridin-4-yloxy)-acetic acid IIy-II-4. To a stirred solution of (3-Aminooxalyl-1-biphenyl-2-ylmethyl-2-methyl-1H-pyrrolo[3,2-c]pyridin-4-yloxy)-acetic acid ethyl ester (7) (30 mg, 0.064 mmol) in THF/EtOH/H₂O (2 mL/2 mL/2 mL), LiOH (16 mg, 0.67 mmol) was added. The reaction mixture was stirred at room temperature for 2 h, evaporated and then acidified (pH=4) with 1 N HCl to form a precipitate, which was filtered off, washed with water and dried in vacuum to afford product IIy-II-4 as a yellow solid.

Yield: 12 mg (43%).

¹H NMR: 05-056-043 (DMSO-d₆, 400 MHz) δ 2.32 (s, 3H), 4.78 (s, 2H), 5.39 (s, 2H), 6.42 (d, 1H), 7.04 (d, 1H), 7.20-7.60 (m, 9H), 7.74 (d, 1H), 7.88 (s, 1H), 12.6 (s, 1H).

MS: 444.02 (M+H).

### Example 16.5

### COMPOUND 2-8

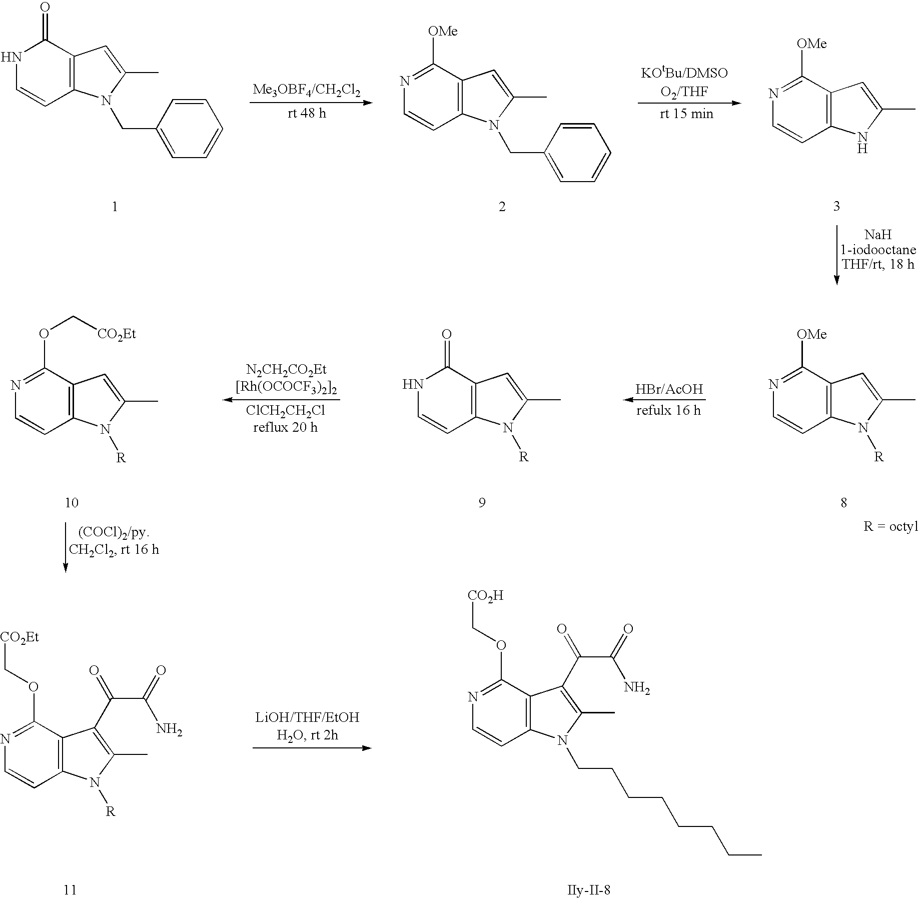

(1-Benzyl-4-methoxy-2-methyl-1H-pyrrolo[3,2-c]pyridine 2. To a stirred suspension of 1-benzyl-2-methyl-1,5-dihydro-pyrrolo[3,2-c]pyridin-4-one 1 (2.0 g, 8.4 mmol) in CH₂Cl₂ (70 mL), Me₃OBF₄ (3.80 g, 25.6 mmol) was added and the reaction mixture was stirred for 48 h, then diluted with CH₂Cl₂ (70 mL). The mixture was washed with water (100 mL), brine (100 mL), dried over Na₂SO₄ and evaporated. Flash chromatography of the residue over silica gel, using 10% EtOAc in hexanes to 25% EtOAc in hexanes) gave product 2 as a pale yellow solid. Yield: 1.6 g (75%).

4-Methoxy-2-methyl-1H-pyrrolo[3,2-c]pyridine 3. To a stirred solution of (1-benzyl-4-methoxy-2-methyl-1H-pyrrolo[3,2-c]pyridine 2 (0.887 mg, 3.52 mmol) in THF (10 mL), DMSO (2.5 mL), followed by KO^{t}Bu (25 mL, 1.0 M in THF) was added dropwise, and then the reaction mixture was treated with O₂ for 15 min at room temperature, quenched with saturated NH₄Cl (20 mL), extracted with EtOAc (3×60 mL). The combined organic extracts were washed with water (50 mL), brine (50 mL), dried over Na₂SO₄ and evaporated. Flash chromatography of the residue over silica gel, using 20% EtOAc in hexanes to 40% EtOAc in hexanes) gave product 3 as a yellow solid. Yield: 560 mg (98%).

4-Methoxy-2-methyl-1-octyl-1H-pyrrolo[3,2-c]pyridine 8. To a stirred suspension of NaH (98 mg, 2.5 mmol, 60% in mineral oil) in THF (10 mL), 4-methoxy-2-methyl-1H-pyrrolo[3,2-c]pyridine 3 (280 mg, 1.72 mmol) in THF (3 mL) was added. The mixture was stirred at room temperature for 30 min, and then 1-iodooctane (0.41 mL, 2.2 mmol) was added, stirring was continued for 18 h. The reaction mixture was quenched with saturated NH₄Cl (20 mL), extracted with EtOAc (3×40 mL). The combined organic extracts were washed with water (40 mL), brine (40 mL), dried over Na₂SO₄ and evaporated. Flash chromatography of the residue over silica gel, using 10% EtOAc in hexanes to 20% EtOAc in hexanes) gave product 8 as a yellow oil. Yield: 231 mg (49%).

2-Methyl-1-octyl-1,5-dihydro-pyrrolo[3,2-c]pyridin-4-one 9. To a stirred solution of 4-methoxy-2-methyl-1-octyl-1H-pyrrolo[3,2-c]pyridine 8 (0.22 g, 0.80 mmol) in AcOH (10 mL), 48% of HBr (5 mL) was added. The reaction mixture was heated to 105° C., and then stirred for 16 h, cooled to room temperature and evaporated. The obtained residue was dissolved in CH₂Cl₂ (80 mL), washed with saturated NaHCO₃ (30 mL), brine (30 mL), dried over Na₂SO₄ and evaporated to afford crude product 9, which was used without further purification for next step. Yield: 207 mg (100%).

(2-Methyl-1-octyl-1H-pyrrolo[3,2-c]pyridin-4-yloxy)-acetic acid ethyl ester 10. To a stirred solution of 2-methyl-1-octyl-1,5-dihydro-pyrrolo[3,2-c]pyridin-4-one 9 (0.207 g, 0.800 mmol) in ClCH₂CH₂Cl (40 mL), [Rh(OCOCF₃)₂]₂ (30 mg, 0.046 mmol) was added, and then a solution of N₂CH₂CO₂Et (0.10 mL. 0.96 mmol) in ClCH₂CH₂Cl (8 mL) was added over 16 h via a syringe pump. The reaction mixture was cooled to room temperature and evaporated. Flash chromatography of the residue over silica gel, using 10% EtOAc in hexanes to 25% EtOAc in hexanes) gave product 10 as a yellow oil. Yield: 70 mg (25%).

(3-Aminooxalyl-2-methyl-1-octyl-1H-pyrrolo[3,2-c]pyridin-4-yloxy)-acetic acid ethyl ester 11. To a stirred solution of (2-methyl-1-octyl-1H-pyrrolo[3,2-c]pyridin-4-yloxy)-acetic acid ethyl ester 10 (68 mg, 0.20 mmol) in CH₂Cl₂ (10 mL), (COCl)₂ (60 μL, 0.68 mmol), followed by pyridine (30 μL) was added dropwise, and then the reaction mixture was stirred at room temperature for 16 h, treated with NH₃ (g) for 30 min and stirred for another 1 h. The precipitated mixture was diluted with EtOAc (40 mL), washed with water (20 mL), brine (20 mL), dried over Na₂SO₄ and evaporated. Flash chromatography of the residue over silica gel, using 50% hexanes in EtOAc to 25% hexanes in EtOAc) gave product 11 as a yellow solid. Yield: 45 mg (55%).

(3-Aminooxalyl-2-methyl-1-octyl-1H-pyrrolo[3,2-c]pyridin-4-yloxy)-acetic acid (IIy-II-8). To a stirred solution of (3-aminooxalyl-2-methyl-1-octyl-1H-pyrrolo[3,2-c]pyridin-4-yloxy)-acetic acid ethyl ester 11 (42 mg, 0.10 mmol) in THF/EtOH/H₂O (3 mL/3 mL/3 mL), LiOH (17 mg, 0.70 mmol) was added. The reaction mixture was stirred at room temperature for 2 h, evaporated and then acidified (pH=4) with 1 N HCl to form a precipitate, which was filtered off, washed with water and dried in vacuum to afford product IIy-II-8 as a yellow solid. Yield: 30 mg (77%).

¹H NMR: 05-056-041 (DMSO-d₆, 400 MHz) δ 0.85 (t, 3H), 1.20-1.40 (m, 10H), 1.55-1.75 (m, 2H), 2.58 (s, 3H), 4.20 (t, 2H), 4.78 (s, 2H), 7.24 (d, 1H), 7.49 (s, 1H), 7.78 (d, 1H), 7.87 (s, 1H), 12.7 (s, 1H).

MS: 390.04 (M+H).

### Example 16.6

### COMPOUND 2-11

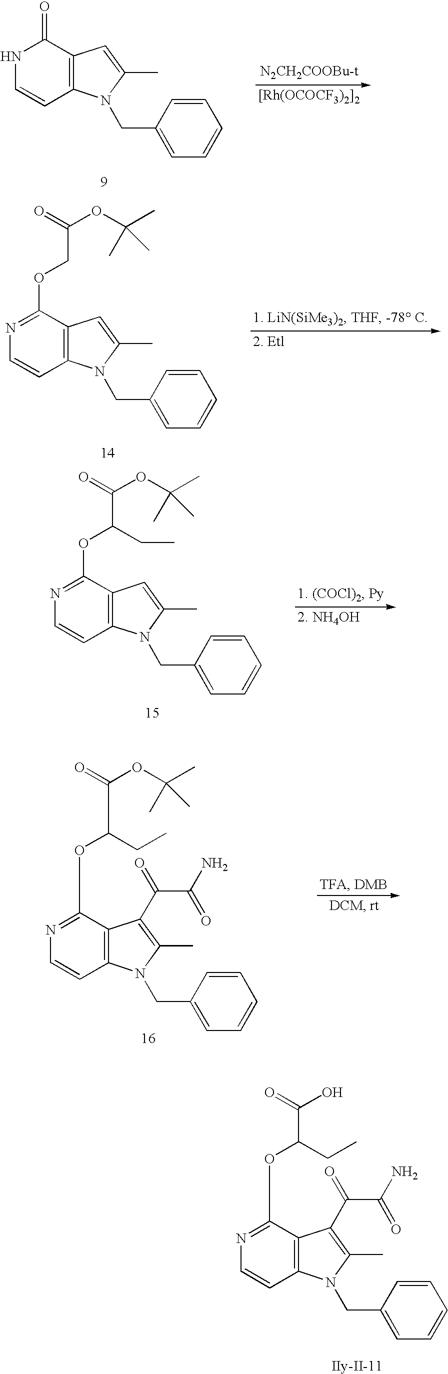

(1-Benzyl-2-methyl-1H-pyrrolo[3,2-c]pyridin-4-yloxy)-acetic acid tert-butyl ester, 14: 1-Benzyl-2-methyl-1,5-dihydro-pyrrolo[3,2-c]pyridin-4-one, 9 (1.0 g, 4.20 mmol) was dissolved in a dry dichloroethane (500 mL). To the mixture Rh₂(OCOCF₃)₄ (132 mg, 0.202 mmol) was added. The reaction mixture was heated to reflux and then to the reaction mixture a solution of tert-butyl diazoacetate (0.65 mL, 4.20 mmol) in dry dichloroethane (50 mL) was added dropwise over 16 h under refluxing. After addition the reaction mixture was stirred for 1 h under refluxing. Then the reaction mixture was cooled to room temperature. The mixture was concentrated and the residue was purified by silica gel chromatography (hexane to hexane:ethyl acetate, 3:1) to afford (1-benzyl-2-methyl-1H-pyrrolo[3,2-c]pyridin-4-yloxy)-acetic acid tert-butyl ester, 14 Yield: 700 mg, (51%)

2-(1-Benzyl-2-methyl-1H-pyrrolo[3,2-c]pyridin-4-yloxy)-butyric acid tert-butyl ester, 15: (1-Benzyl-2-methyl-1H-pyrrolo[3,2-c]pyridin-4-yloxy)-acetic acid tert-butyl ester, 14 (200 mg, 0.568 mmol) was dissolved in a dry tetrahydrofuran (10 mL) and then cooled to −78° C. To the mixture the tetrahydrofuran solution (1.0 M) of LiN(Si(CH₃)₃)₂ (1.70 mL) was added dropwise at −78° C. The reaction mixture was stirred from −78° C. to −5° C. for 1 h and then the tetrahydrofuran solution (5 mL) of iodoethane (0.15 mL, 1.84 mmol) was added dropwise at −50° C. The mixture was stirred for 4 h from −50° C. to room temperature. The mixture was concentrated and the residue was purified by silica gel chromatography (hexane to hexane:ethyl acetate, 4:1) to afford 2-(1-benzyl-2-methyl-1H-pyrrolo[3,2-c]pyridin-4-yloxy)-butyric acid tert-butyl ester, 15 Yield: 50 mg, (23%)

2-(3-Aminooxalyl-1-benzyl-2-methyl-1H-pyrrolo[3,2-c]pyridin-4-yloxy)-butyric acid tert-butyl ester, 16: 2-(1-benzyl-2-methyl-1H-pyrrolo[3,2-c]pyridin-4-yloxy)-butyric acid tert-butyl ester, 15 (134 mg, 0.352 mmol) was dissolved in a dry chloroform (10 mL). To the mixture the solution of oxalyl chloride (0.10 mL, 1.13 mmol) in chloroform (5 mL) was added dropwise at room temperature. Then pyridine (0.05 mL) was added slowly to the mixture at room temperature. After addition the mixture was stirred at room temperature for 18 h. The mixture was poured into icy 20% NH₄OH solution (100 mL) and stirred for 1 h. The mixture was diluted with dichloromethane (20 mL). The organic layer was separated and aqueous layer was extracted with dichloromethane (2×20 mL). The organic layers were combined and dried over anhydrous MgSO₄. The mixture was filtered. The filtrate was concentrated and the residue was purified by silica gel chromatography (hexan to hexane:ethyl acetate, gradient 1:1) to afford 2-(3-aminooxalyl-1-benzyl-2-methyl-1H-pyrrolo[3,2-c]pyridin-4-yloxy)-butyric acid tert-butyl ester, 16 as a yellow solid. Yield: 62 mg, (39%)

2-(3-Aminooxalyl-1-benzyl-2-methyl-1H-pyrrolo[3,2-c]pyridin-4-yloxy)-butyric acid, IIy-II-11: 2-(3-aminooxalyl-1-benzyl-2-methyl-1H-pyrrolo[3,2-c]pyridin-4-yloxy)-butyric acid tert-butyl ester, 16 (26 mg, 0.0576 mmol) was dissolved in dichloromethane (2 mL). To the mixture 1,3-dimethoxybenzene (0.023 mL, 0.172 mmol) was added at room temperature. The mixture was cooled to 0° C. for 30 min. To the mixture trifluoroacetic acid (0.015 mL, 0.234 mmol) was added at 0° C. After addition the mixture was stirred at 0° C. for 1 h. Then mixture was warmed up to room temperature and stirred for 2 h at room temperature. Then more trifluoroacetic acid (0.1 mL) was added and the mixture was stirred at room temperature for 18 h. The mixture was concentrated and H-NMR indicated the reaction was not completed. The residue was redissolved in dichloromethane (5 mL) and then trifluoroacetic acid (0.5 mL) was added at room temperature. The mixture was stirred at room temperature for 6 h. The mixture was concentrated and the residue was purified by silica gel preparative thin layer chromatography (hexane:ethyl acetate, 1:1) to afford 2-(3-aminooxalyl-1-benzyl-2-methyl-1H-pyrrolo[3,2-c]pyridin-4-yloxy)-butyric acid, IIy-II-11 as a light yellow solid. Yield: 11 mg, (48%)

¹H NMR: 05-43-128-2, (400 MHz, DMSO-d6)

δ, 8.09 (br, s, 1H, NH), 7.72 (d, 1H), 7.54 (br, s, 1H, NH), 7.20-7.38 (m, 3H), 7.18 (d, 1H), 7.08 (d, 2H), 5.50 (br, s, 2H, PhCH₂N), 5.02 (t, 1H, CHOAr), 2.41 (br, s, 3H, Me), 1.92 (q, 2H, Et), 1.02 (t, 3H, Et), ppm.

MS (ES): 395.98 [M+1].

### Example 16.7

### COMPOUND (2-9)

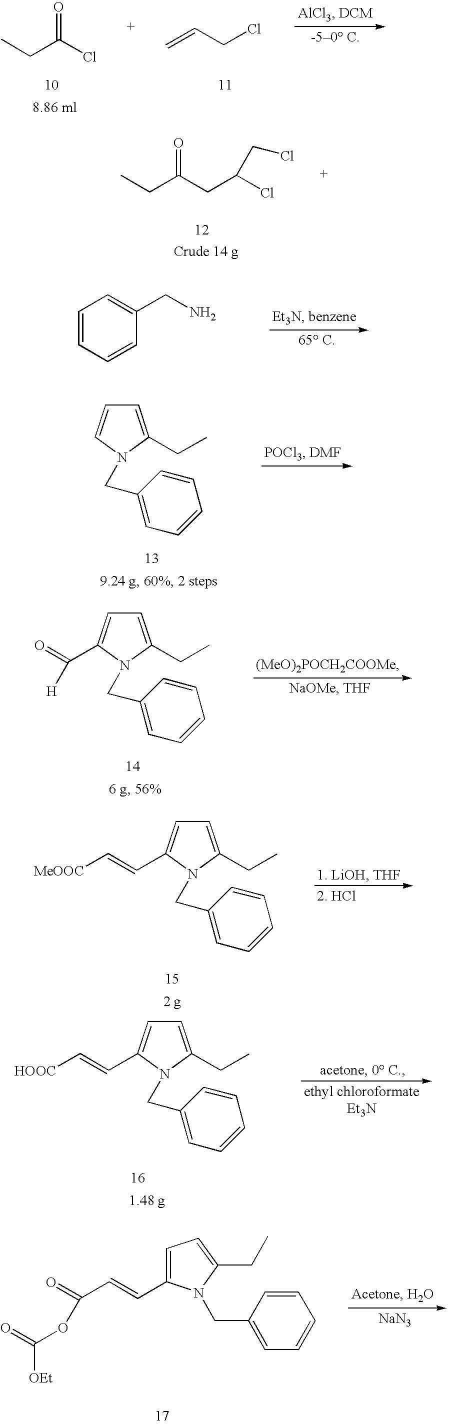

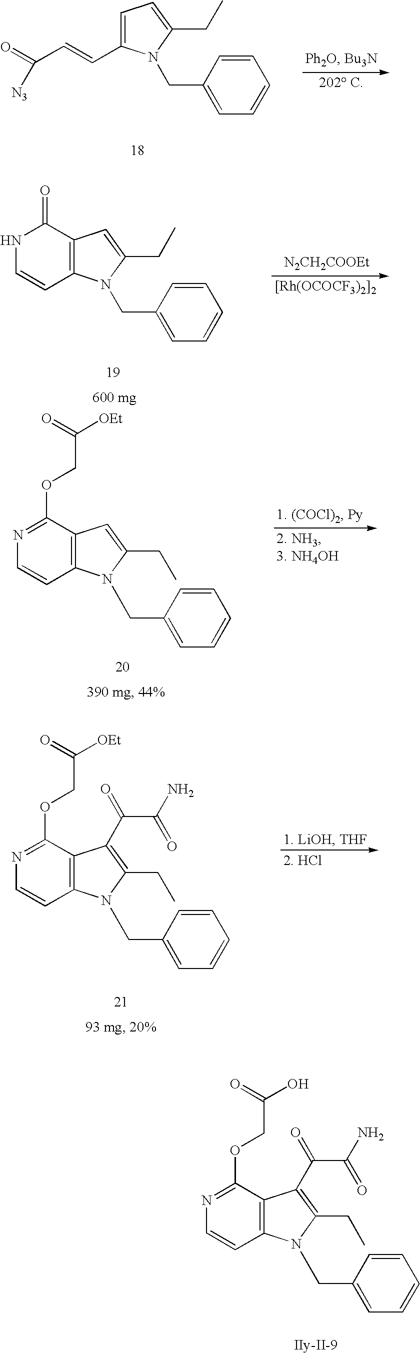

2-(3-(2-amino-2-oxoacetyl)-1-benzyl-2-ethyl-1H-pyrrolo[3,2-c]pyridin-4-yloxy)acetic acid (ILY-II-9)

5,6-dichlorohexan-3-one,12 To a solution of propionyl chloride (8.86 mL, 102 mmol) and ally chloride (115 mmol) in dichloromethane (500 mL) at −5° C. aluminum chloride (115 mmol) was added. The resulted solution was stirred for 5 hr, then was allowed to warmed up to 0° C. After evaporating solvent the residue was extracted by ether (3×150 mL). The combined extracts was washed with water (2×200 mL), followed by removing solvent and drying to give 14 g of crude 12.

1-benzyl-2-ethyl-1H-pyrrole, 13: To the crude 12 (14 g, 83 mmol) in dry benzene (200 mL) at room temperature was added benzylamine solution (12.5 mL, 100 mmol) and triethylamine (11 g, 110 mmol). The solution was heated to reach 65° C. and stirred for 18 h. The resulted reaction mixture was filtered and concentrated. The crude product was purified by silica gel chromatography to afford 1-benzyl-2-ethyl-1H-pyrrole 13 (9.24 g (50 mmol), 60% for two step).

1-benzyl-5-ethyl-1H-pyrrole-2-carbaldehyde, 14: POCl₃ (23.46 mL, 246 mmol) was added dropwise to a stirred N,N-dimethylformamide (204 mL) at 0° C. After addition the mixture was stirred for additional 90 minutes. To the mixture was added dropwise the solution of 1-benzyl-2-ethyl-1H-pyrrole, 13 (8.33 g, 45 mmol) in tetrahydrofuran (1150 mL). The reaction mixture was allowed to be stirred for 18 h from 0° C. to room temperature. The mixture was concentrated and redissolved in ethyl acetate (2 L). The mixture was washed with saturated Na₂CO₃ (2×500 mL). The Na₂CO₃ solution was extracted with ethyl acetate (7×1 L). The organic layers were combined and concentrated. The crude product was purified by silica gel chromatography (hexane to hexane:ethyl acetate, 7:1) to afford 1-benzyl-5-ethyl-1H-pyrrole-2-carbaldehyde, 14 Yield: 6 g (56%).

(E)-methyl 3-(1-benzyl-5-ethyl-1H-pyrrolo-2-yl)acrylate, 15: Sodium (0.75 g, 32 mmol) was added in portions to a dry methanol (30 mL). To the fresh formed sodium methoxide solution was added dropwise the solution of trimethyl phosphonoacetate (2.6 mL, 15.2 mmol) in tetrahydrofuran (7 mL) at room temperature. After addition the mixture was stirred for additional 60 min at room temperature. Then to the reaction mixture was added dropwise the solution of 1-benzyl-5-ethyl-1H-pyrrole-2-carbaldehyde, 14 (2 g) in tetrahydrofuran (50 mL) at room temperature. The reaction mixture was stirred for 2 h at room temperature. The mixture was concentrated and redissolved in ethyl acetate (200 L). The mixture was washed with 1 M HCl solution, then saturated NaHCO₃, H₂O. The organic solution were dried over MgSO₄ and then filtered, concentrated to afford the crude product, (E)-methyl 3-(1-benzyl-5-ethyl-1H-pyrrolo-2-yl)acrylate, 15. Yield: 2 g

(E)-3-(1-benzyl-5-ethyl-1H-pyrrolo-2-yl)acrylic acid, 16: (E)-methyl 3-(1-benzyl-5-ethyl-1H-pyrrolo-2-yl)acrylate, 15 (2 g) was dissolved in a mixture of tetrahydrofuran (40 mL) and methanol (40 mL). To the mixture a solution of lithium hydroxide monohydrate (1 g, 25 mmol) in H₂O (20 mL) was added. After addition the reaction mixture was stirred for 18 h at room temperature. The reaction mixture was acidified by 2M HCl to pH=4-5. The mixture was concentrated and redissolved in ethyl acetate. The mixture was washed with H₂O. The water layer was extracted with ethyl acetate (2×250 mL). The organic was combined and concentrated to afford a yellow solid which was washed with dichloromethaneto, followed by purification on silica gel chromatography (hexane to hexane:ethyl acetate, 1:3, followed by neat ethyl acetate) to afford (E)-3-(1-benzyl-5-ethyl-1H-pyrrolo-2-yl)acrylic acid, 16 (1.48 g).

1-benzyl-2-ethyl-1H-pyrrolo[3,2-c]pyridin-4(5H)-one, 19: 3(E)-3-(1-benzyl-5-ethyl-1H-pyrrolo-2-yl)acrylic acid, 16 (1.48 g, 5.8 mmol) was dissolved in a dry acetone (70 mL). To the suspension mixture triethylamine (1.9 mL) was added to form a clear solution. The reaction mixture was cooled to 0° C. and then to the cooled reaction mixture a solution of ethyl chloroformate (16 mmol) in dry acetone (65 mL) was added dropwise over 1 hour. After addition the reaction mixture was stirred for 4 h at 0° C. Then to the reaction mixture was added dropwise the solution of sodium azide (770 mg, 11.7 mmol) in H₂O (17 mL) over 30 minutes. The reaction mixture was stirred at 0° C. for 2 h. The reaction mixture was poured into ice-water (500 mL). Then the mixture was extracted with dichloromethane (3×250 mL). The organic layers were combined and dried over MgSO₄. The mixture was filtered and concentrated to afford a crude 18. To the mixture of diphenyl ether (17 mL) and tributylamine (1.65 mL) which was preheated to 205° C. was added dropwise the solution of crude 18 in diphenyl ether (25 mL) at 205° C. for 1 hour. After addition the mixture was stirred for another hour at 205° C. The mixture was cooled to room temperature and solid was formed. Diethyl ether (50 mL) was added into the reaction mixture to form more solid. The mixture was filtered and the solid was washed with diethyl ether to afford the product. The filtrate was concentrated and the residue was purified by silica gel chromatography to afford 1-benzyl-2-ethyl-1H-pyrrolo[3,2-c]pyridin-4(5H)-one, 19 (600 mg).

ethyl 2-(1-benzyl-2-ethyl-1H-pyrrolo[3,2-c]pyridin-4-yloxy)acetate, 20: 1-benzyl-2-ethyl-1H-pyrrolo[3,2-c]pyridin-4(5H)-one, 19 (600 mg, 2.38 mmol) was dissolved in a dry dichloroethane (300 mL). To the mixture Rh₂(OCOCF₃)₄ (71 mg, 0.103 mmol) was added. The reaction mixture was heated to reflux and then to the reaction mixture a solution of ethyl diazoacetate (2.37 mmol) in dry dichloroethane (30 mL) was added dropwise over 6 h under refluxing. After addition the reaction mixture was stirred for 1.5 h under refluxing. Then the reaction mixture was cooled to room temperature. The mixture was concentrated and the residue was purified by silica gel chromatography to afford ethyl 2-(1-benzyl-2-ethyl-1H-pyrrolo[3,2-c]pyridin-4-yloxy)acetate, 20. Yield: 390 mg, (44%)

ethyl 2-(3-(2-amino-2-oxoacetyl)-1-benzyl-2-ethyl-1H-pyrrolo[3,2-c]pyridin-4-yloxy)acetate, 21: ethyl 2-(1-benzyl-2-ethyl-1H-pyrrolo[3,2-c]pyridin-4-yloxy)acetate, 20 (390 mg, 1.15 mmol) was dissolved in a dry chloroform (37 mL). To the mixture the solution of oxalyl chloride (0.30 mL, 3.45 mmol) in chloroform (10 mL) was added dropwise at room temperature. Then pyridine (0.140 mL) was added slowly to the mixture at room temperature. After addition the mixture was stirred at room temperature for 18 h. The mixture was concentrated and the residue was purified by silica gel chromatography to afford ethyl 2-(3-(2-amino-2-oxoacetyl)-1-benzyl-2-ethyl-1H-pyrrolo[3,2-c]pyridin-4-yloxy)acetate, 21 Yield: 93 mg, (20%)

2-(3-(2-amino-2-oxoacetyl)-1-benzyl-2-ethyl-1H-pyrrolo[3,2-c]pyridin-4-yloxy)acetic acid, IIy-II-9: ethyl 2-(3-(2-amino-2-oxoacetyl)-1-benzyl-2-ethyl-1H-pyrrolo[3,2-c]pyridin-4-yloxy)acetate, 21 (93 mg, 0.227 mmol) is dissolved in methanol (20 mL). To the mixture the solution of KOH (1M, 0.25 mL) is added at room temperature. After addition the mixture was stirred at room temperature for 18 h. Then solution of lithium hydroxide monohydrate (90 mg) in H₂O (5 mL) is added. After another hour stirring the mixture was concentrated and the residue is redissolved in methanol (10 mL) and ethanol (10 mL). The mixture is filtered and the filtrate was acidified by hydrogen chloride in ether (1.0 M) to pH=34. Solvent is evaporated and the residue is washed with a mixture of dichloromethane:ether (1:1), then water (5 mL) and ether to afford 2-(3-(2-amino-2-oxoacetyl)-1-benzyl-2-ethyl-1H-pyrrolo[3,2-c]pyridin-4-yloxy)acetic acid, IIy-II-9.

### Example 16.8

### COMPOUND (2-10)

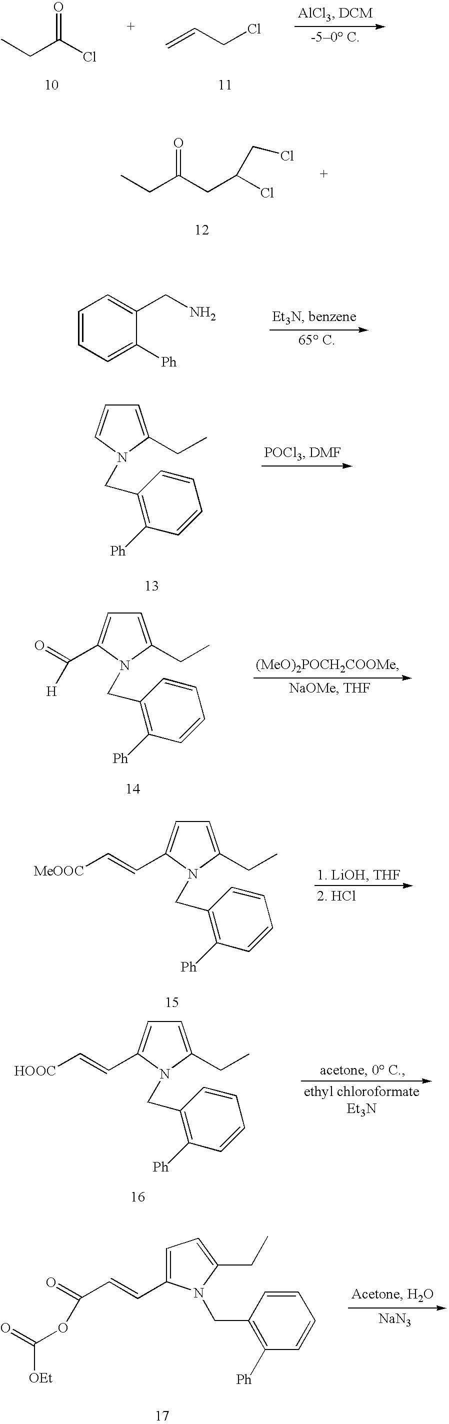

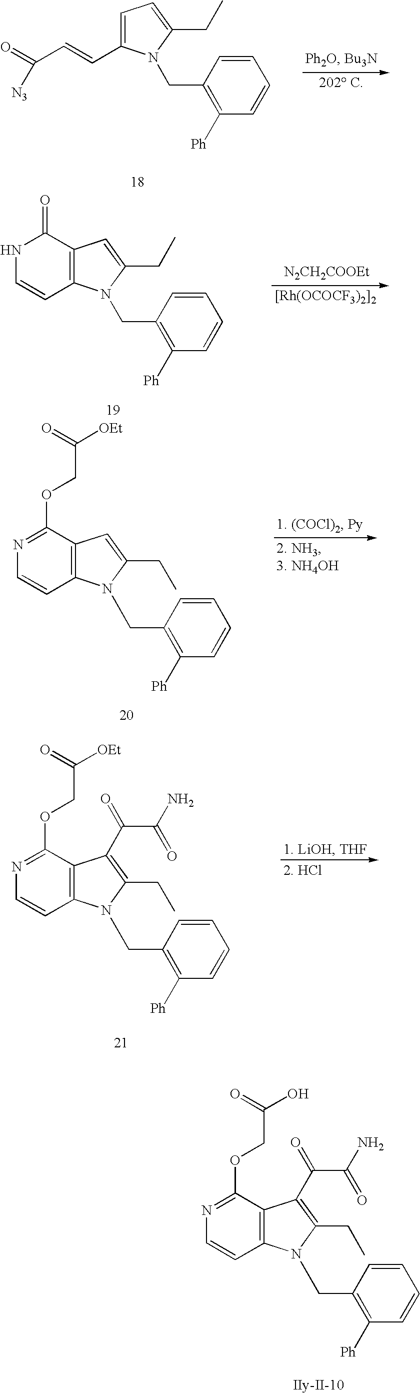

2-(3-(2-amino-2-oxoacetyl)-1-(biphenyl-2-ylmethyl)-2-ethyl-1H-pyrrolo[3,2-c]pyridin-4-yloxy)acetic acid (ILY-II-10)

5,6-dichlorohexan-3-one,12 To a solution of propionyl chloride (8.86 mL, 102 mmol) and ally chloride (115 mmol) in dichloromethane (500 mL) at −5° C. aluminum chloride (115 mmol) was added. The resulted solution was stirred for 5 hr, then was allowed to warmed up to 0° C. After evaporating solvent the residue was extracted by ether (3×150 mL). The combined extracts was washed with water (2×200 mL), followed by removing solvent and drying to give 14 g of crude 12.

1-(biphenyl-2-ylmethyl)-2-ethyl-1H-pyrrole, 13: To the crude 12 (14 g, 83 mmol) in dry benzene (200 mL) at room temperature is added biphenyl-2-ylmethanamine solution (100 mmol) and triethylamine (110 mmol). The solution is heated to reach 65° C. and stirred for 18 h. The resulted reaction mixture is filtered and concentrated. The crude product was purified by silica gel chromatography to afford 13

1-(biphenyl-2-ylmethyl)-5-ethyl-1H-pyrrole-2-carbaldehyde, 14: POCl₃ (23.46 mL, 246 mmol) is added dropwise to a stirred N,N-dimethylformamide (204 mL) at 0° C. After addition the mixture is stirred for additional 90 minutes. To the mixture is added dropwise the solution of 13 (45 mmol) in tetrahydrofuran (1150 mL). The reaction mixture was allowed to be stirred for 18 h from 0° C. to room temperature. The mixture was concentrated and redissolved in ethyl acetate (2 L). The mixture was washed with saturated Na₂CO₃ (2×500 mL). The Na₂CO₃ solution was extracted with ethyl acetate (7×1 L). The organic layers were combined and concentrated. The crude product was purified by silica gel chromatography to afford 14.

(E)-methyl 3-(1-(biphenyl-2-ylmethyl)-5-ethyl-1H-pyrrolo-2-yl)acrylate, 15: Sodium (0.75 g, 32 mmol) is added in portions to a dry methanol (30 mL). To the fresh formed sodium methoxide solution is added dropwise the solution of trimethyl phosphonoacetate (2.6 mL, 15.2 mmol) in tetrahydrofuran (7 mL) at room temperature. After addition the mixture is stirred for additional 60 min at room temperature. Then to the reaction mixture is added dropwise the solution of 14 (2 g) in tetrahydrofuran (50 mL) at room temperature. The reaction mixture is stirred for 2 h at room temperature. The mixture is concentrated and redissolved in ethyl acetate (200 mL). The mixture is washed with 1 M HCl solution, then saturated NaHCO₃, H₂O. The organic solution is dried over MgSO₄ and then filtered, concentrated to afford the crude product 15.

(E)-3-(1-(biphenyl-2-ylmethyl)-5-ethyl-1H-pyrrolo-2-yl)acrylic acid, 16: Compound 15 (2 g) is dissolved in a mixture of tetrahydrofuran (40 mL) and methanol (40 mL). To the mixture a solution of lithium hydroxide monohydrate (1 g, 25 mmol) in H₂O (20 mL) is added. After addition the reaction mixture is stirred for 18 h at room temperature. The reaction mixture is acidified by 2M HCl to pH=4-5. The mixture is concentrated and redissolved in ethyl acetate. The mixture is washed with H₂O. The water layer is extracted with ethyl acetate (2×250 mL). The organic is combined and concentrated to afford a yellow solid which is washed with dichloromethaneto, followed by purification on silica gel chromatography to afford 16.

1-(biphenyl-2-ylmethyl)-2-ethyl-1H-pyrrolo[3,2-c]pyridin-4(5H)-one, 19: Compound 16 (5.8 mmol) is dissolved in a dry acetone (70 mL). To the suspension mixture triethylamine (1.9 mL) is added to form a clear solution. The reaction mixture is cooled to 0° C. and then to the cooled reaction mixture a solution of ethyl chloroformate (16 mmol) in dry acetone (65 mL) is added dropwise over 1 hour. After addition the reaction mixture is stirred for 4 h at 0° C. Then to the reaction mixture is added dropwise the solution of sodium azide (770 mg, 11.7 mmol) in H₂O (17 mL) over 30 minutes. The reaction mixture is stirred at 0° C. for 2 h. The reaction mixture is poured into ice-water (500 mL). Then the mixture is extracted with dichloromethane (3×250 mL). The organic layers are combined and dried over MgSO₄. The mixture is filtered and concentrated to afford a crude 18. To the mixture of diphenyl ether (17 mL) and tributylamine (1.65 mL) which is preheated to 205° C. is added dropwise the solution of crude 18 in diphenyl ether (25 mL) at 205° C. for 1 hour. After addition the mixture is stirred for another hour at 205° C. The mixture is cooled to room temperature and solid is formed. Diethyl ether (50 mL) is added into the reaction mixture to form more solid. The mixture is filtered and the solid is washed with diethyl ether to afford the product. The filtrate is concentrated and the residue was purified by silica gel chromatography to afford 19.

ethyl 2-(1-(biphenyl-2-ylmethyl)-2-ethyl-1H-pyrrolo[3,2-c]pyridin-4-yloxy)acetate, 20: Compound 19 (2.38 mmol) is dissolved in a dry dichloroethane (300 mL). To the mixture Rh₂(OCOCF₃)₄ (71 mg, 0.103 mmol) is added. The reaction mixture is heated to reflux and then to the reaction mixture a solution of ethyl diazoacetate (2.37 mmol) in dry dichloroethane (30 mL) is added dropwise over 6 h under refluxing. After addition the reaction mixture is stirred for 1.5 h under refluxing. Then the reaction mixture is cooled to room temperature. The mixture is concentrated and the residue is purified by silica gel chromatography to afford 20.

ethyl 2-(3-(2-amino-2-oxoacetyl)-1-(biphenyl-2-ylmethyl)-2-ethyl-1H-pyrrolo[3,2-c]pyridin-4-yloxy)acetate, 21: Compound 20 (1.15 mmol) is dissolved in a dry chloroform (37 mL). To the mixture the solution of oxalyl chloride (0.30 mL, 3.45 mmol) in chloroform (10 mL) is added dropwise at room temperature. Then pyridine (0.140 mL) is added slowly to the mixture at room temperature. After addition the mixture is stirred at room temperature for 18 h. The mixture is concentrated and the residue is purified by silica gel chromatography to afford 21.

2-(3-(2-amino-2-oxoacetyl)-1-(biphenyl-2-ylmethyl)-2-ethyl-1H-pyrrolo[3,2-c]pyridin-4-yloxy)acetic acid, IIy-II-10: Compound 21 (0.227 mmol) is dissolved in methanol (20 mL). To the mixture the solution of KOH (1M, 0.25 mL) is added at room temperature. After addition the mixture was stirred at room temperature for 18 h. Then solution of lithium hydroxide monohydrate (90 mg) in H₂O (5 mL) is added. After another hour stirring the mixture was concentrated and the residue is redissolved in methanol (10 mL) and ethanol (10 mL). The mixture is filtered and the filtrate was acidified by hydrogen chloride in ether (1.0 M) to pH=3-4. Solvent is evaporated and the residue is washed with a mixture of dichloromethane:ether (1:1), then water (5 mL) and ether to afford 2-(3-(2-amino-2-oxoacetyl)-1-benzyl-2-ethyl-1H-pyrrolo[3,2-c]pyridin-4-yloxy)acetic acid, IIy-II-10.

### Example 16.9a

### COMPOUND (2-12)

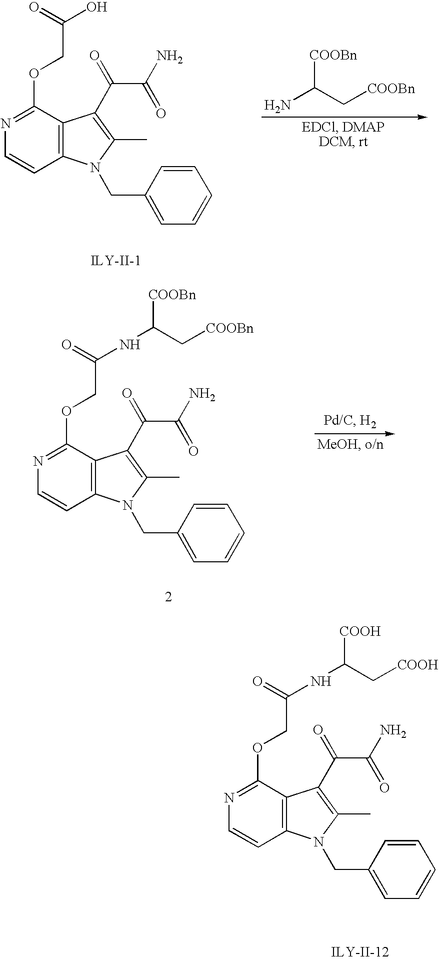

2-(2-(3-(2-amino-2-oxoacetyl)-1-benzyl-2-methyl-1H-pyrrolo[3,2-c]pyridin-4-yloxy)acetamido)succinic acid (ILY-II-12)

To a solution of 2-(3-(2-amino-2-oxoacetyl)-1-benzyl-2-methyl-1H-pyrrolo[3,2-c]pyridin-4-yloxy)acetic acid ILY-II-1 (1.5 mmol) in dichloromethane/dimethylformamide (5:1) is added aspartic acid dibenzyl ester (313 mg 1.5 mmol), 4-dimethylaminopyridine (18 mg 0.15 mmol), 1-hydroxybenzotriazole (202 mg, 1.5 mmol) and 1-(3-dimethylaminopropyl)-3-ethylcarbodiimide hydrochloride (286 mg, 1.5 mmol), respectively and reaction mixture allows to stir at room temperature. After 6 hrs the reaction is diluted with dichloromethane and washed twice with 1N HCl and brine. The organic layer is dried with Na₂SO₄ and evaporated in vacuum. The residue is chromatographed on silica gel to give the titled compound 2.

A solution of 2 (0.25 mmol) in ethanol 10 mL is stirred in hydrogen atmosphere using a balloon in the presence of Pd/C 50 mg. After stirring for 18 h the catalyst was filtered through celite and solvent is evaporated to give the target compound (2-(2-(3-(2-amino-2-oxoacetyl)-1-benzyl-2-methyl-1H-pyrrolo[3,2-c]pyridin-4-yloxy)acetamido)succinic acid) ILY-II-12.

### Example 16.9b

### COMPOUND (2-12)

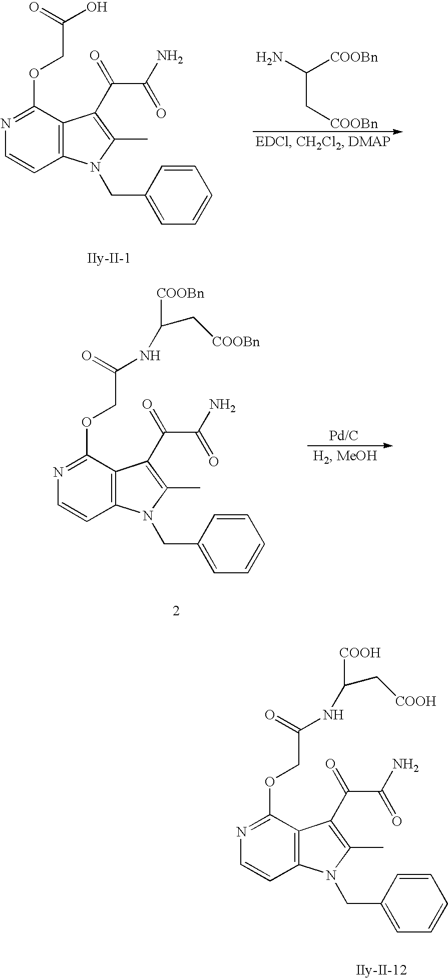

### 3-[2-(7-Aminooxalyl-5-benzyl-6-methyl-5H-[2]pyrimidin-1-yloxy)-acetylamino]-pentanedioic acid dibenzyl ester (2)

To a mixture of IIy-II-1 (0.052 g, 0.177 mmole) in dichloromethane (10 mL) DMAP (0.045 g, 0.354 mmole), L-aspartic acid dibenzyl ester p-toluenesulfonate (0.173 g, 0.354 mmole), EDCI (0.068 g, 0.354 mmole) and HBTU (0.048 g, 0.354 mmole) were added. The mixture was stirred at room temperature for 18 h. The reaction mixture was diluted with dichloromethane (50 mL). The organic layer was washed with 1M HCl (50 mL), then water (50 mL). The organic layer was separated, dried over magnesium sulphate and concentrated. The residue was purified by column chromatography (4:1 EtOAc:Hexane) to afford intermediate (2) as a yellow solid. Yield: 0.03 g, 26%.

2-[2-(3-Aminooxalyl-1-benzyl-2-methyl-1H-pyrrolo[3,2-c]pyridin-4-yloxy)-acetylamino]-malonic acid (IIy-II-12): To a solution of intermediate (2) (0.040 g, 0.0604 mmole) in methanol (10 mL) Pd/C (10%, 0.015 g) was added. Hydrogen was passed through the mixture at 1 atm and room temperature for 1.5 h. The reaction mixture was filtered through Celite. The filtrate was concentrated to afford a white solid which was dissolved in methanol (10 mL) and passed through a PTFE filter. The filtrate was concentrated to afford IIy-II-12 as a yellow solid. Yield: 0.020 g, 68%. ¹H NMR: 05-043-146-2 (DMSO-d₆, 400 MHz) δ, ppm: 8.15-8.05 (m, 2H), 7.22 (d, 1H), 7.35-7.22 (m, 4H), 7.07 (d, 2H), 5.58 (s, 2H), 5.20 (d, 1H), 4.80 (d, 1H), 4.30 (br, 1H), 2.55 (s, 3H). ES-MS: m/z=482.94 (M+1). Compound (2-12)

### Example 16.10

### COMPOUND (2-13)

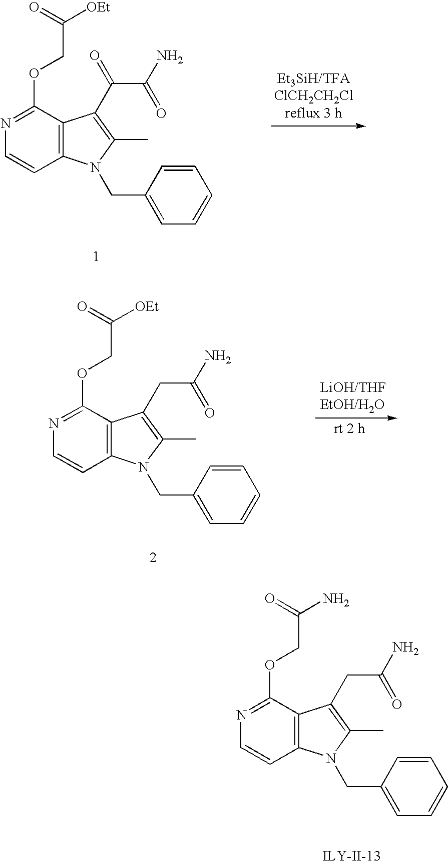

### 2-(4-(2-amino-2-oxoethoxy)-1-benzyl-2-methyl-1H-pyrrolo[3,2-c]pyridin-3-yl)acetamide (ILY-II-13)

To a stirred solution of ILY-II-1 ethyl ester 1 (0.22 mmol) in dichloroethane (7 mL), Et₃SiH (0.5 mL) and CF₃CO₂H (0.5 mL) are added. The mixture is heated to 85° C. and stirring is continued for 3 h. The reaction mixture is cooled to room temperature and evaporated. The obtained residue is diluted with EtOAc (50 mL), washed with cold saturated NaHCO₃ (20 mL), brine (20 mL), dried over Na₂SO₄ and evaporated to give crude product 2, which is used without further purification for the next step.

To a stirred solution of 1 (0.22 mmol) in THF/EtOH/H₂O (3 mL/3 mL/3 mL), LiOH (53 mg, 2.2 mmol) is added. The reaction mixture is stirred at room temperature for 2 h, evaporated and then acidified (pH=4) with 1 N HCl to form a precipitate, which is filtered off, washed with water and dried in vacuum to afford product IIy-II-13.

### Example 16.11a

### COMPOUND (2-14)

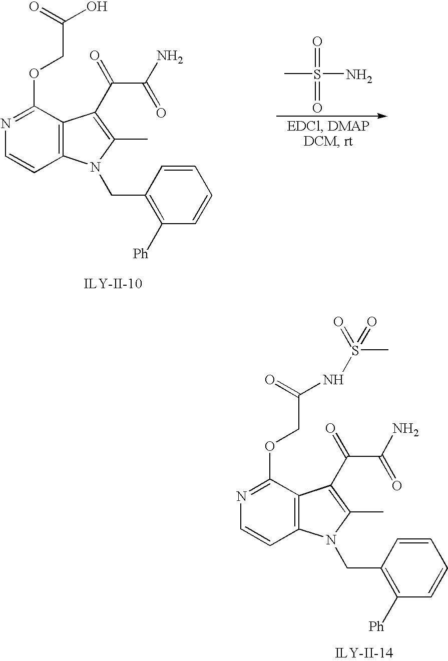

2-(3-(2-amino-2-oxoacetyl)-1-(biphenyl-2-ylmethyl)-2-methyl-1H-pyrrolo[3,2-c]pyridin-4-yloxy)-N-(methylsulfonyl)acetamide (ILY-II-14) To a solution of 2-(3-(2-amino-2-oxoacetyl)-1-(biphenyl-2-ylmethyl)-2-ethyl-1H-pyrrolo[3,2-c]pyridin-4-yloxy)acetic acid, IIy-II-10 (2.3 mmol) in dichloromethane/dimethylformamide mixture (4:1, 10 mL) is added 4-dimethylaminopyridine (3.4 mmol), methanesulfonamide (431 mg, 4.5 mmol) and 1-(3-dimethylaminopropyl)-3-ethylcarbodiimide hydrochloride (433 mg, 2.3 mmol) and the reaction mixture is stirred at room temperature. After 24 h the reaction mixture is diluted with dichloromethane and washed twice with 1N HCl and brine. The organic layer is dried with Na₂SO₄ and evaporated in vacuum. The residue is chromatographed on silica gel (CHCl₃ to 4% MeOH in CHCl₃) to give 2-(3-(2-amino-2-oxoacetyl)-1-(biphenyl-2-ylmethyl)-2-methyl-1H-pyrrolo[3,2-c]pyridin-4-yloxy)-N-(methylsulfonyl)acetamide (ILY-II-14).

### Example 16.11b

### COMPOUND (2-14)

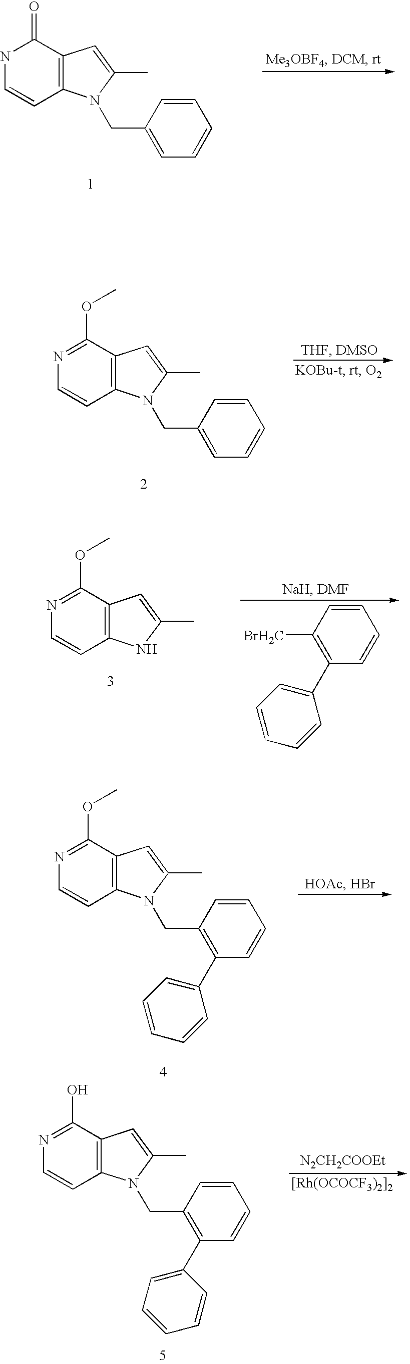

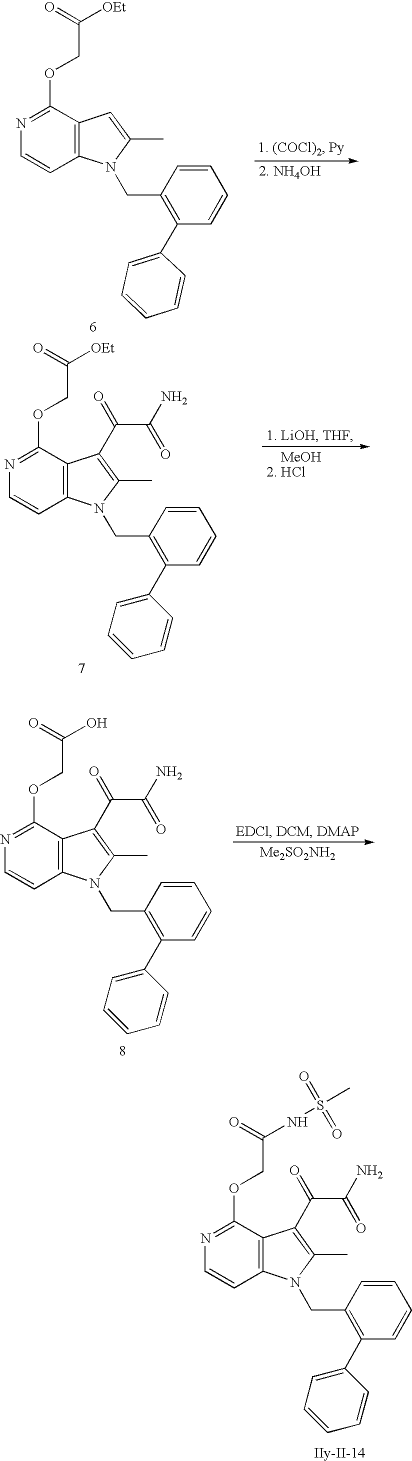

1-Benzyl-4-methoxy-2-methyl-1H-pyrrolo[3,2-c]pyridine (2): To a mixture of 1-benzyl-2-methyl-1,5-dihydro-pyrrolo[3,2-c]pyridin-4-one (1) (3.48 g, 16.62 mmole) in dichloromethane (160 mL) trimethyloxonium tetrafluoroborate (4.52 g, 29.24 mmole) was added. The mixture was stirred at room temperature for 18 h. Additional trimethyloxonium tetrafluoroborate (2.25 g, 14.55 mmole) was added and stirred for 18 h. The mixture was filtered and the filtrate was concentrated. The residue was purified by column chromatography (20:1 CH₂Cl₂:MeOH) to afford intermediate (2) Yield: 1.31 g, 35%.

4-Methoxy-2-methyl-1H-pyrrolo[3,2-c]pyridine (3): To a solution of 1-benzyl-4-methoxy-2-methyl-1H-pyrrolo[3,2-c]pyridine (2) (0.887 g, 3.51 mmole) in anhydrous THF (10 mL) dimethyl sulfoxide (25 mL) was added slowly (via a syringe) and the mixture was cooled to 0° C. Potassium tert-butoxide (1M in THF, 25 mL, 25 mmole) was added slowly. Oxygen was bubbled through the mixture for 45 minutes. The reaction was quenched with saturated ammonium chloride solution, the mixture was extracted with ethyl acetate (3×50 mL). The organic layer was separated, dried over magnesium sulphate and concentrated. The residue was purified by column chromatography (3:1 Hex:EtOAc) to afford intermediate (3). Yield: 1.06 g >100%

1-Biphenyl-2-ylmethyl-4-methoxy-2-methyl-1H-pyrrolo[3,2-c]pyridine (4): To a solution of intermediate (3) (0.70 g, 4.69 mmole) in anhydrous DMF (40 mL) sodium hydride (60% in mineral oil, 0.28 g, 7.04 mmole) was added, the mixture was stirred for 1 h. To the mixture 2-phenylbenzyl bromide (0.95 mL, 5.16 mmole) was added dropwise. The mixture was stirred at room temperature for 18 h. The reaction was quenched with saturated ammonium chloride solution (200 mL), the mixture was extracted with ethyl acetate (3×200 mL). The organic layer was separated and washed with water, dried over magnesium sulphate and concentrated. The residue was purified by column chromatography (3:1 Hex:EtOAc) to afford intermediate (4) as a yellow solid. Yield: 1.1 g 71%.

1-Biphenyl-2-ylmethyl-2-methyl-1H-pyrrolo[3,2-c]pyridin-4-ol (5): To a solution of intermediate (4) in acetic acid (45 mL) hydrogen bromide (48% solution, 15 mL) was added. The mixture was heated at 105° C. for 18 h. The reaction mixture was concentrated, then dissolved in dichloromethane (100 mL) and washed with ammonium chloride solution (100 mL). The organic layer was separated, dried over magnesium sulphate and concentrated to afford intermediate (5) as a solid. Yield: 0.65 g, 62%.

(1-Biphenyl-2-ylmethyl-2-methyl-1H-pyrrolo[3,2-c]pyridin-4-yloxy)-acetic acid ethyl ester (6): To a solution of intermediate (5) (0.557 g, 1.77 mmole) in 1,2-dichloroethane (250 mL) rhodium (II) trifluoroacetate dimmer (0.058 g, 0.0885 mmole) was added, the reaction was heated to reflux. Then the solution of ethyl diazoacetate (90%, 0.2 mL, 1.77 mmole) in dichloroethane (35 mL) was added via a syringe pump to the mixture over 16 h. The reaction mixture was refluxed for an additional 2 h. The solvent was evaporated and the residue was purified by column chromatography (3:1 Hex:EtOAc) to afford intermediate (6) as a yellow solid. Yield: 0.272 g, 38%.

(3-Aminooxalyl-1-biphenyl-2-ylmethyl-2-methyl-1H-pyrrolo[3,2-c]pyridin-4-yloxy)-acetic acid ethyl ester (7): To a solution of intermediate (6) (0.255 g, 0.637 mmole) in chloroform (20 mL) oxalyl chloride (0.169 mL, 1.898 mmole) in chloroform (6 mL) was added dropwise, followed by the addition of pyridine (0.1 mL). The mixture was stirred at room temperature for 18 h. The reaction mixture was poured onto ice cold ammonium chloride solution (50 mL), dichloromethane (50 mL) was added and the mixture was stirred for 1 h. The organic layer was separated and the aqueous layer was further extracted with chloroform (3×50 mL). The organic fractions were combined, dried over magnesium sulphate and concentrated. The residue was purified by column chromatography (3:1 EtOAc:Hex) to afford intermediate (7) as a yellow solid. Yield: 0.18 g, 60%)

(3-Aminooxalyl-1-biphenyl-2-ylmethyl-2-methyl-1H-pyrrolo[3,2-c]pyridin-4-yloxy)-acetic acid (8): To a solution of intermediate (7) (0.18 g, 0.382 mmole) in THF/MeOH (10 mL/10 mL) lithium hydroxide monohydrate (0.035 g, 0.852 mmole) was added. The reaction mixture was stirred at room temperature for 1.5 h. The mixture was acidified to pH 1-2 with 2M HCl, then adjusted to pH=6.5 with 1 M KOH solution. The solvent was evaporated and the water was decanted off. The residue was washed with water (2×5 mL), followed by diethyl ether (2×5 mL). The solid was collected by filtration and dried under high vacuum to afford intermediate (8) as a yellow solid. Yield: 0.13 g, 72%.

2-[1-Biphenyl-2-ylmethyl-4-(2-methanesulfonylamino-2-oxo-ethoxy)-2-methyl-1H-pyrrolo[3,2-c]pyridin-3-yl]-2-oxo-acetamide (IIy-II-14): To a mixture of intermediate (8) (0.13 g, 0.295 mmole) in dichloromethane (10 mL) DMAP (0.065 g, 0.45 mmole), methanesulfonamide (0.056 g, 0.58 mmole) and 1-(3-dimethylaminopropyl)-3-ethylcarbodiimide hydrochloride (EDAC) (0.056 g, 0.293 mmole) were added. The mixture was stirred at room temperature for 18 h. The solvent was concentrated and the residue was purified by preparative TLC (10:1 CH₂Cl₂:MeOH) afford to IIy-II-14. Yield: 0.035 g, 23%. ¹H NMR: 05-043-167-2a (DMSO-d₆, 400 MHz) δ, ppm: 7.96 (s, 1H), 7.75 (d, 1H), 7.60-7.22 (m, 10H), 7.02 (d, 1H), 6.42 (d, 1H), 5.40 (s, 2H), 4.75 (s, 2H), 3.00 (s, 3H), 2.30 (s, 3H). ES-MS: m/z=520.95 (M+1).

Certain such azaindole and azaindole related compounds were evaluated for phospholipase activity using the protocol of Example 8. The results are shown in Table 8.

TABLE 8
Inhibition of pancreas secreted human, mouse and porcine PLA₂

| | | | | | | ILYPSA % | | |
|---|---|---|---|---|---|---|---|---|
| | | | ILYPSA IC50 (μM) | | | inhibition at 15 μM | | |
| | Compound | | | | mps | hps | pps | mps |
| structure | ID | MW | hps PLA₂ | pps PLA₂ | PLA₂ | PLA₂ | PLA₂ | PLA₂ |
| 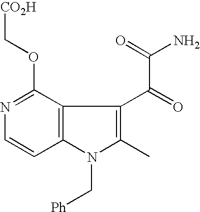 | ILY-II-1 (2-1) | 367.36 | 1.15 | 0.07 | 0.23 | | | |
| 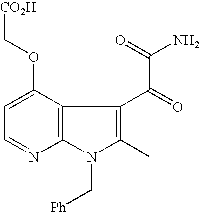 | ILY-VII-1 (7-1) | 367.36 | 13.65 | 0.06 | 2.14 | | | |
| 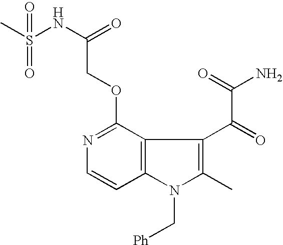 | ILY-II-7 (2-7) | 444.46 | 2.07 | 0.04 | 1.05 | | | |
| 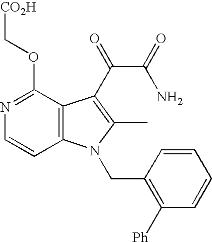 | ILY-II-4 (2-4) | 443.45 | 0.08 | <0.02 | 0.07 | | | |
| 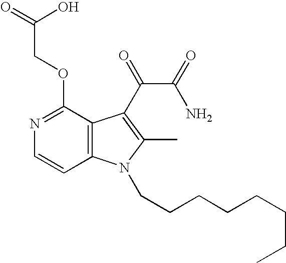 | ILY-II-8 (2-8) | 369.45 | 0.27 | 0.08 | 0.12 | | | |
| 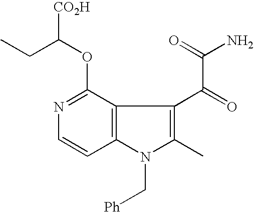 | ILY-II-11 (2-11) | 395.41 | 0.48 | <0.02 | 0.03 | | | |
| 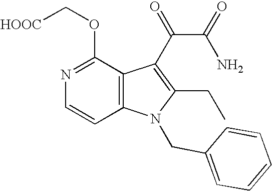 | ILY-II-9 (2-9) | 381.38 | 1.46 | 0.03 | 0.35 | | | |
| 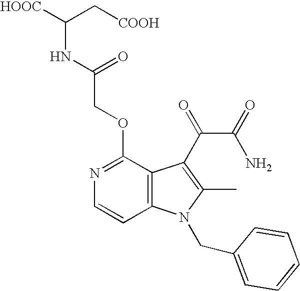 | ILY-II-12 (2-12) | 482.44 | | 3.71 | | 16.63 | | 35.68 |
| 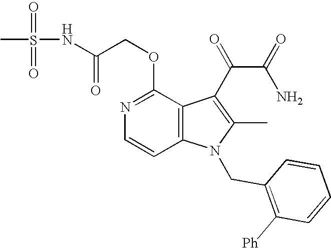 | ILY-II-14 (2-14) | 520.57 | 0.69 | 0.04 | 0.59 | | | |

### Example 17

### PREPARATION OF PHOSPHOLIPASE INHIBITING MOIETIES

This example demonstrated the synthesis of various compounds suitable for use as phospholipase inhibiting moieties.

### Example 17A

### COMPOUND 3-1

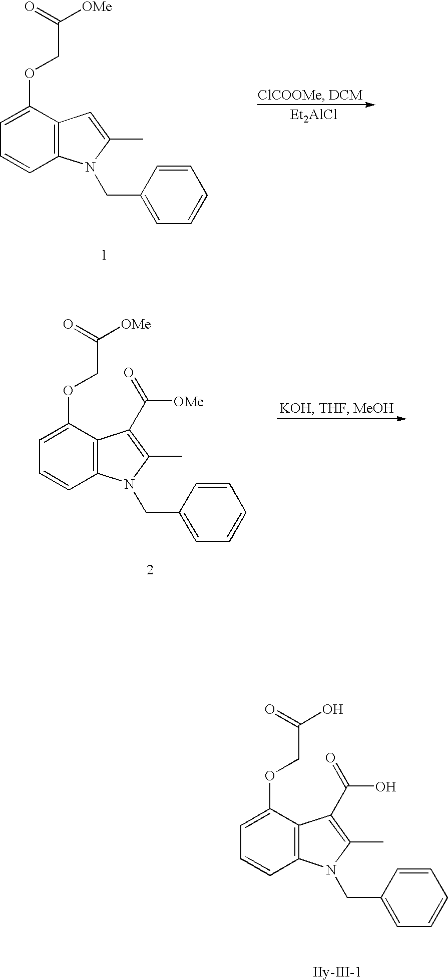

1-Benzyl-4-methoxycarbonylmethoxy-2-methyl-1H-indole-3-carboxylic acid methyl ester, 2 (intermediate): (1-Benzyl-2-methyl-1H-indol-4-yloxy)-acetic acid methyl ester, 1 (700 mg, 2.26 mmol) was dissolved in dichloromethane (15 mL). The mixture was cooled to 0° C. To the mixture diethylaluminum chloride solution (1.0 M in hexane, 12 mL) was added dropwise at 0° C. After addition the mixture was stirred at 0° C. for 30 minutes. The solution of methyl chloroformate (0.9 mL, 10 mmol) in dichloromethane (15 mL) was added dropwise to the reaction mixture at 0° C. Then reaction mixture solution was stirred at 0° C. for 2 h. The reaction was stopped by adding water. The reaction mixture was diluted with adding more dichloromethane. The organic layer was washed with water and dried over Na₂SO₄. The mixture was filtered. The filtrate was concentrated and the residue was purified by silica gel chromatography (hexane to hexane:ethyl acetate, gradient 1:3 to 1:1) to afford 1-benzyl-4-methoxycarbonylmethoxy-2-methyl-1H-indole-3-carboxylic acid methyl ester, 2 as an off-white solid.

Yield: 540 mg, (65%)

1-Benzyl-4-methoxycarbonylmethoxy-2-methyl-1H-indole-3-carboxylic acid, IIy-III-1 (Compound 3-1): 1-Benzyl-4-methoxycarbonylmethoxy-2-methyl-1H-indole-3-carboxylic acid methyl ester, 2 (540 mg, 1.47 mmol) was dissolved in a mixture of tetrahydrofuran (3 mL) and methanol (3 mL). To the mixture the solution of KOH (10.0 M, 3 mL) was added at room temperature. After addition the mixture was stirred at room temperature for 18 h. The mixture was acidified by concentrated HCl to pH=1-2. Solvent was evaporated and the residue was extracted with ethyl acetate. The organic solution was washed with water and dried over MgSO₄. The mixture was filtered and filtrate was concentrated. The residue was washed with a mixture of methanol:ethyl acetate (1:1) and ether to afford 1-Benzyl-4-methoxycarbonylmethoxy-2-methyl-1H-indole-3-carboxylic acid, IIy-III-1 as an off-white solid. Yield: 98 mg, (20%)

¹H NMR: 04-73-230-5, (400 MHz, DMSO-d6)

δ, 7.20-7.39 (m, 4H), 7.14 (t, 1H), 7.02 (q, 2H), 6.72 (q, 1H), 5.57 (br, s, 2H, PhCH₂N), 4.86 (br, s, 2H, CH₂OAr), 2.62 (s, 3H, CH₃) ppm.

MS (ES): 337.91 [M−1].

### Example 17B

### COMPOUND 5-5

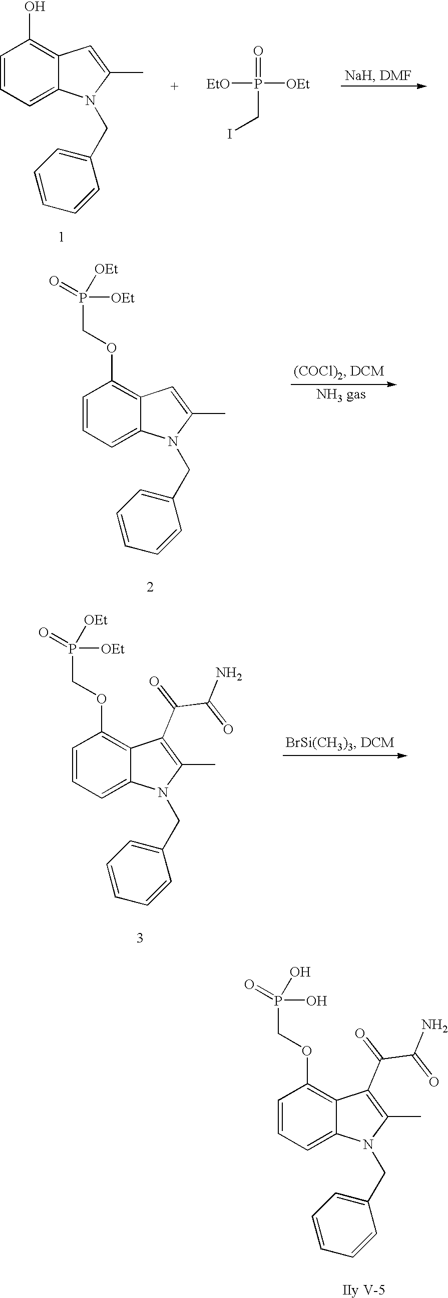

(1-Benzyl-2-methyl-1H-indol-4-yloxymethyl)-phosphonic acid diethyl ester, 2 (intermediate): 1-Benzyl-2-methyl-1H-indol-4-ol 1 (0.3 g, 1.26 mmole) was dissolved in anhydrous dimethylformamide (20 mL). To the mixture sodium hydride 60% in mineral oil (66 mg, 1.65 mmole) was added. The mixture was stirred at room temperature for 30 minutes. To the mixture diethyliodomethylphosphate (0.35 mL, 1.65 mL) was added. The mixture was stirred at room temperature for 18 h. The reaction was diluted with ethyl acetate (300 mL) and washed with H₂O (5×100 mL) and brine (100 mL). The organic layer was separated and concentrated. The residue was purified by column chromatography (3:1 EtOAc:Hex). The resulting brown liquid (0.38 g) was a mixture 2:1 diethyliodomethylphosphate:2. The material was used without further purification in the subsequent step.

(3-Aminooxalyl-1-benzyl-2-methyl-1H-indol-4-yloxymethyl)-phosphonic acid diethyl ester, 3 (intermediate): (1-Benzyl-2-methyl-1H-indol-4-yloxymethyl)-phosphonic acid diethyl ester 2 (0.19 g, 0.34 mmole) was dissolved in anhydrous dichloromethane (25 mL). To the mixture oxalyl chloride (0.045 mL, 0.51 mmole) was added. The reaction mixture was stirred at room temperature for 1 h. NH₃ gas was then bubbled through the solution for 30 minutes and the mixture stirred at room temperature for 1 h. The dichloromethane was evaporated. The residue was dissolved in ethyl acetate (300 mL) and washed with H₂O (3×100 mL) and brine (100 mL). The organic layer was separated, dried with magnesium sulfate and concentrated to afford 3 (0.15 g, 96%).

(3-Aminooxalyl-1-benzyl-2-methyl-1H-indol-4-yloxymethyl)-phosphonic acid IIy-V-5 (Compound 5-5): (3-Aminooxalyl-1-benzyl-2-methyl-1H-indol-4-yloxymethyl)-phosphonic acid diethyl ester 3 (0.15 g, 0.327 mmole) was dissolved in anhydrous dichloromethane (10 mL). To the mixture bromotrimethylsilane (0.33 mL, 2.55 mmole) was added. The mixture stirred at room temperature for 18 h. The reaction mixture was evaporated. The residue was stirred in acetonitrile (5 mL), diethyl ether (5 mL) and H₂O (3 drops). The resulting precipitate was collected by filtration to afford IIy-V-5 (0.022 g, 17%) as a brown solid.

¹H NMR (DMSO) δ 7.85 (s, 1H), 7.50 (s, 1H), 7.35-7.25 (m, 3H), 7.15-7.00 (m, 4H), 6.92 (d, 2H), 5.50 (s, 2H), 4.25 (d, 2H), 2.45 (s, 3H). MS (ES+) 402.95.

### Example 17C

### COMPOUND 4-3

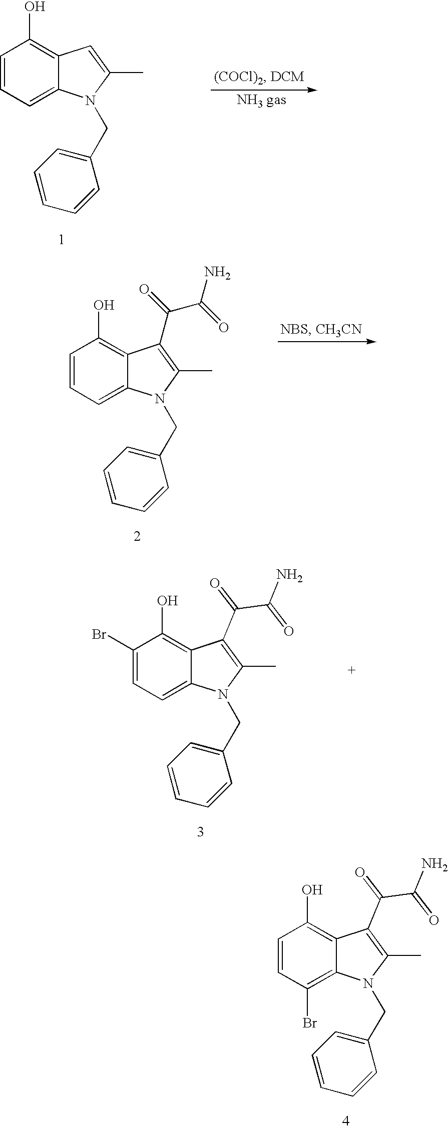

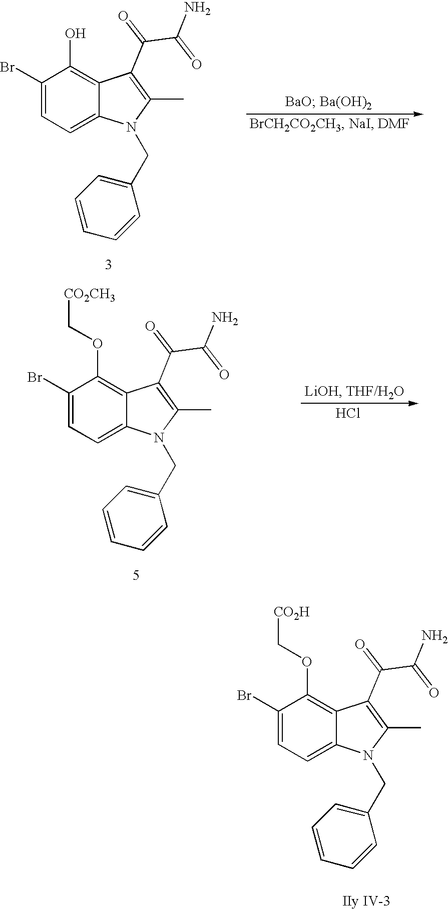

2-(1-Benzyl-4-hydroxy-2-methyl-1H-indol-3yl)-2-oxo-acetamide 2: To a solution of oxalyl chloride (2.16 mL, 24.8 mmole) in anhydrous dichloromethane (100 mL) a solution of 1-Benzyl-2-methyl-1H-indol-4-ol 1 (2.80 g, 11.8 mmole) in anhydrous dichloromethane (100 mL) was added drop-wise. The mixture was left to stir at room temperature for 1 h. NH₃ gas was then bubbled through the mixture for 1 h. The mixture was left to stir at room temperature for 18 h. The dichloromethane was evaporated. The residue was dissolved in ethyl acetate (1 L) and washed with H₂O (4×500 mL) and brine (500 mL). The organic layer was separated, dried with magnesium sulfate and concentrated to afford 2 (2.0 g, 55%) as a yellow solid.

2-(1-Benzyl-5-bromo-4-hydroxy-2methyl-1H-indol-3-yl)-2-oxo-acetamide 24 and 2-(1-Benzyl-7-bromo-4-hydroxy-2methyl-1H-indol-3-yl)-2-oxo-acetamide 3: 2-(1-Benzyl-4-hydroxy-2-methyl-1H-indol-3yl)-2-oxo-acetamide 2 (5.0 g, 16.23 mmole) was mixed in anhydrous acetonitrile (700 mL). To the mixture N-bromosuccinimide (2.87 g, 16.23 mmole) was added. The mixture was stirred at room temperature for 2.5 h. The acetonitrile was evaporated. The residue was dissolved in ethyl acetate (2 L) and washed with H₂O (3×1 L) and brine (1 L). The organic layer was separated, dried with magnesium sulfate and concentrated to a volume of approximately (300 mL). To the mixture methanol (50 mL) was added and the mixture cooled to room temperature. The resulting precipitate was collected by filtration and washed with diethyl ether to afford 3 (3.35 g, 53%) as an orange solid, approximately 90% pure. The filtrate was evaporated and the residue was purified by column chromatography (3:1 EtOAc:Hex) to afford 4 (0.5 g, 8%) as a yellow solid.

(3-Aminooxalyl-1-benzyl-5-bromo-2-methyl-1H-indol-4-yloxy)-acetic acid methyl ester 5: 2-(1-Benzyl-5-bromo-4-hydroxy-2methyl-1H-indol-3-yl)-2-oxo-acetamide 3 (0.1 g, 0.26 mmole) was dissolved in anhydrous dimethylformamide (20 mL). To the mixture barium oxide (0.08 g, 0.52 mmole), barium hydroxide hydrate (0.08 g, 0.257 mmole) and sodium iodide (20 mg) were added. The mixture was stirred at room temperature for 30 minutes. To the mixture methyl-2-bromoacetate (0.04 mL, 0.4 mmole) was added. The reaction stirred at room temperature for 4 h. The reaction was diluted with ethyl acetate (500 mL) and washed with H₂O (5×150 mL) and brine (150 mL). The organic layer was separated, dried with magnesium sulfate and concentrated to afford 5 (0.11 g, 93%) as an orange solid.

(3-Aminooxalyl-1-benzyl-5-bromo-2-methyl-1H-indol-4-yloxy)-acetic acid, IIy-IV-3 (Compound 4-3): (3-Aminooxalyl-1-benzyl-5-bromo-2-methyl-1H-indol-4-yloxy)-acetic acid methyl ester 5 (0.1 g, 0.217 mmole) was mixed in THF:H₂O 4:1 (10 mL). To the mixture lithium hydroxide monohydrate (0.015 g, 0.38 mmole) was added. The mixture stirred at room temperature for 30 min. The reaction mixture was evaporated to dryness under high vacuum. The residue was dissolved in H₂O (5 mL). The solution was acidified with 2M HCl. The resulting precipitate was collected by filtration, washed with H₂O and diethyl ether and dried to afford IIy-IV-3 (0.042 g, 43%) as a yellow solid.

Ref: 04-090-181.1: ¹H NMR (DMSO) δ 12.70 (s, broad, 1H), 7.88 (s, 1H), 7.60 (s, 1H), 7.40-7.20 (m, 5H), 7.05 (d, 2H), 5.55 (s, 2H), 4.60 (s, 2H), 2.50 (s, 3H). MS (ES+) 444.94, 446.96

### Example 17D

### COMPOUND 4-9

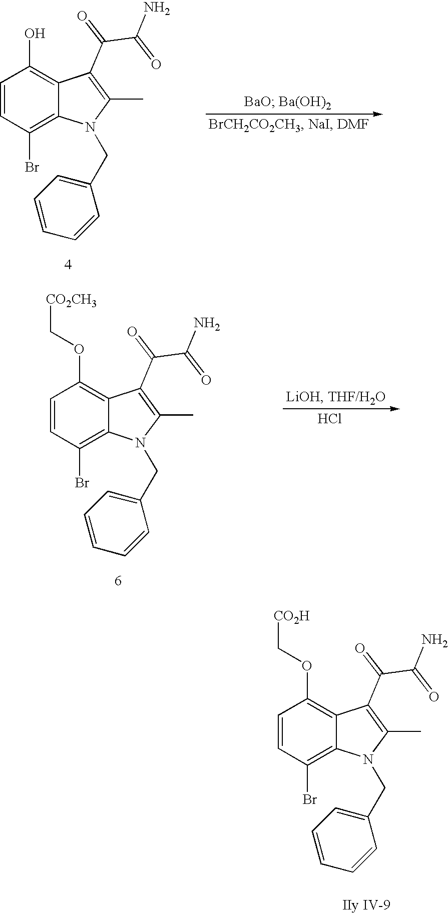

(3-Aminooxalyl-1-benzyl-7-bromo-2-methyl-1H-indol-4-yloxy)-acetic acid methyl ester 6: 2-(1-Benzyl-7-bromo-4-hydroxy-2methyl-1H-indol-3-yl)-2-oxo-acetamide 4 (0.257 g, 0.66 mmole) was dissolved in anhydrous dimethylformamide (20 mL). To the mixture barium oxide (0.08 g, 1.3 mmole), barium hydroxide octahydrate (0.08 g, 0.642 mmole) and sodium iodide (40 mg) were added. The mixture was stirred at room temperature for 30 minutes. To the mixture methyl-2-bromoacetate (0.04 mL, 0.4 mmole) was added. The mixture was stirred at room temperature for 18 h. The reaction was diluted with ethyl acetate (500 mL) and washed with H₂O (5×150 mL) and brine (150 mL). The organic layer was separated, dried with magnesium sulfate and concentrated. The residue was washed with ethyl acetate and collected by filtration to afford 6 (0.146 g, 48%) as an orange solid.

(3-Aminooxalyl-1-benzyl-7-bromo-2-methyl-1H-indol-4-yloxy)-acetic acid IIy-IV-9 (Compound 4-9): (3-Aminooxalyl-1-benzyl-7-bromo-2-methyl-1H-indol-4-yloxy)-acetic acid methyl ester 6 (0.19 g 0.414 mmole) was stirred in THF:H₂O 4:1 (10 mL). To the mixture lithium hydroxide monohydrate (0.1 g, 2.53 mmole) was added. The mixture was stirred at room temperature for 30 minutes. The reaction mixture was evaporated to dryness and the residue was dissolved in H₂O (5 mL) and acidified with 2M HCl. The mixture was stirred for 1 h. The resulting precipitate was collected by filtration and washed with H₂O and diethyl ether to afford IIy-IV-9 (0.106 g, 57%) as an orange solid.

Ref: 04-090-215.1: ¹H NMR (DMSO) δ 13.00 (s, broad, 1H), 7.78 (s, 1H), 7.48 (s, 1H), 7.38-7.20 (m, 4H), 6.92 (d, 2H), 6.52 (d, 1H), 5.90 (s, 2H), 4.70 (s, 2H), 2.40 (s, 3H).

### Example 17E

### COMPOUND 4-16

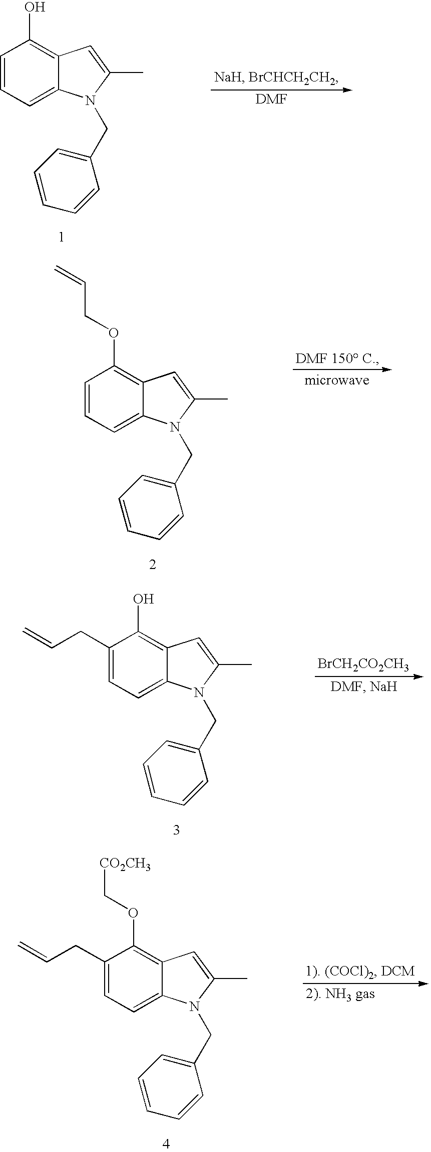

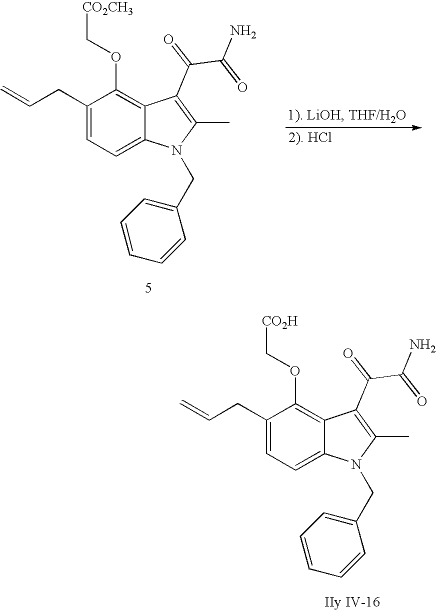

4-Allyloxy-1-benzyl-2-methyl-1H-indole 2: 1-Benzyl-2-methyl-1H-indol-4-ol 1 (2.0 g, 8.43 mmole) was dissolved in anhydrous dimethylformamide (200 mL). To the mixture sodium hydride 60% in mineral oil (0.45 g, 10.9 mmole) was added. The mixture was stirred at room temperature for 1 h. To the mixture allyl bromide (0.94 mL, 10.9 mmole) was added, the mixture was left to stir at room temperature for 18 h. ¹H NMR indicated the reaction was complete. The reaction mixture was diluted with ethyl acetate (700 mL) and washed with H₂O (5×150 mL) and Brine (1×150 mL). The organic layer was separated, dried with magnesium sulfate and concentrated to afford 2 (2.3 g, 98%) as an orange solid.

5-Allyl-1-benzyl-2-methyl-1H-indol-4-ol 3: 4-Allyloxy-1-benzyl-2-methyl-1H-indole 2 (2.3 g, 8.3 mmole) was dissolved in anhydrous dimethylformamide (40 mL). The solution was placed in a sealed tube. The reaction vessel was subjected to 150° C. at 35 psi for 40 minutes in a microwave reactor. The reaction mixture was diluted with ethyl acetate (400 mL) and washed with H₂O (5×100 mL) and Brine (1×100 mL). The organic layer was separated, dried with magnesium sulfate and concentrated to afford 3 (2.3 g, 100%) as an orange oil.

(5-Allyl-1-benzyl-2-methyl-1H-indol-4-yloxy-acetic acid methyl ester 4: 5-Allyl-1-benzyl-2-methyl-1H-indol-4-ol 3 (2.3 g, 8.3 mmole) was dissolved in anhydrous dimethylformamide (100 mL). To the reaction mixture sodium hydride 60% in mineral oil (0.4 g, 9.96 mmole) was added. The mixture was left to stir at room temperature for 1 h. To the mixture methyl bromo acetate (0.915 mL, 9.96 mmole) was added. The mixture was left to stir at room temperature for 48 h. The reaction mixture was diluted with ethyl acetate (400 mL) and washed with H₂O (5×100 mL) and brine (1×100 mL). The organic layer was separated and concentrated. The residue was purified by column chromatography (6:1 Hexane:EtOAc) to afford 4 (1.8 g, 62%) as an orange oil.

(5-Allyl-3-aminooxalyl-1-benzyl-2-methyl-1H-indol-4-yloxy)-acetic acid methyl ester 5: To a solution of oxalyl chloride (0.26 mL, 3.00 mmole) in anhydrous dichloromethane (100 mL) a solution methyl (5-Allyl-1-benzyl-2-methyl-1H-indol-4-yloxy-acetic acid methyl ester 4 (1.0 g, 2.86 mmole) in anhydrous dichloromethane (100 mL) was added drop-wise. The mixture was left to stir at room temperature for 1 h. To the mixture NH₃ gas was bubbled for 30 minutes. The mixture was left to stir at room temperature for 2 h. The dichloromethane was evaporated and the residue was dissolved in ethyl acetate (300 mL). The organic layer was washed with H₂O (3×300 mL) and brine (1×200 mL). The organic was separated, dried with magnesium sulfate and concentrated to afford 5 (1.1 g, 91%) as a yellow solid.

(5-Allyl-3-aminooxalyl-1-benzyl-2-methyl-1H-indol-4-yloxy)-acetic acid IIy-IV-16 (Compound 4-16): (5-Allyl-3-aminooxalyl-1-benzyl-2-methyl-1H-indol-4-yloxy)-acetic acid methyl ester 5 (0.29 g, 069 mmole) was dissolved in THF:H₂O 4:1 (10 mL). To the mixture lithium hydroxide monohydrate (0.13 g, 3.01 mmole) was added. The mixture was stirred at room temperature for 30 min. The solution was acidified with 2M HCl and stirred at room temperature for 1 h. The THF was evaporated the resulting solid was collected by filtration and washed with diethyl ether to afford IIy-IV-16 (0.19 g, 68%) as a yellow solid.

Ref: 04-090-217: ¹H NMR (DMSO) δ 12.50 (s, broad, 1H), 7.90 (s, 1H), 7.58 (s, 1H), 7.40-7.20 (m, 4H), 7.10-6.90 (m, 3H), 5.95 (m, 1H), 5.50 (s, 2H), 5.00 (m, 2H), 4.35 (s, 2H), 3.50 (m, 2H), 2.50 (s, 3H). MS (ES+) 407.05

### Example 17F

### COMPOUND 4-27

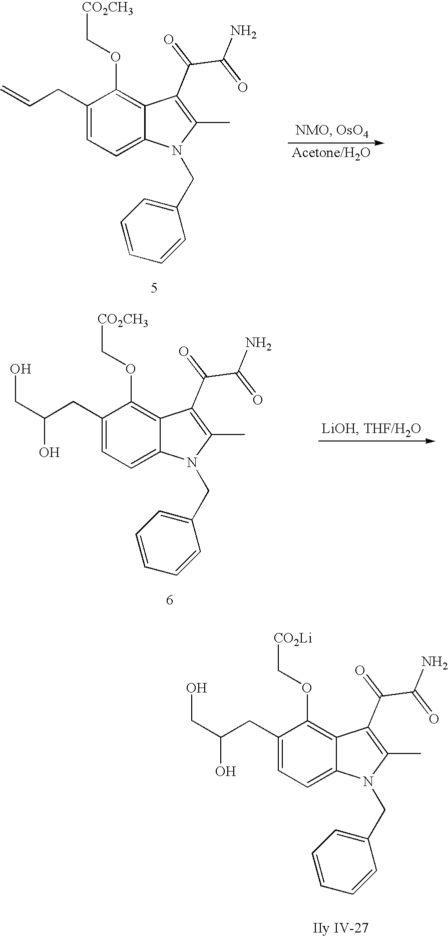

[3-Aminooxalyl-1-benzyl-5-(2,3-dihydroxy-propyl)-2-methyl-1H-indol-4-yloxy]-acetic acid methyl ester 6: (5-Allyl-3-aminooxalyl-1-benzyl-2-methyl-1H-indol-4-yloxy)-acetic acid methyl ester 5 (0.3 g, 0.712 mmole) was dissolved in acetone:H₂O (20 mL). To the mixture N-methylmorpholine N-oxide (0.1 g, 0.815 mmole) and osmium tetroxide (5 grains) was added. The mixture stirred at room temperature for 18 h. The reaction mixture was evaporated, dissolved in ethyl acetate and washed with H₂O. The organic layer was separated, dried with magnesium sulfate and concentrated to afford 6 (0.15 g, 46%) as a solid.

[3-Aminooxalyl-1-benzyl-5-(2,3-dihydroxy-propyl)-2-methyl-1H-indol-4-yloxy]-acetic acid (lithium salt) IIy-IV-27: [3-Aminooxalyl-1-benzyl-5-(2,3-dihydroxy-propyl)-2-methyl-1H-indol-4-yloxy]-acetic acid methyl ester 6 (0.072 g, 0.158 mmole) was dissolved in THF:H₂O (10 mL). To the mixture 0.1705 M LiOH solution (0.929 mL, 0.158 mmole) was added. The mixture stirred at room temperature for 30 min. The reaction mixture was evaporated to dryness under high vacuum. The residue was stirred in diethyl ether and collected by filtration to afford IIy-IV-27 (0.041 g, 58%) as a yellow solid.

Ref: 04-090-250: ¹H NMR (DMSO) δ 8.32 (s, 1H), 7.50-7.00 (m, 8H), 5.45 (s, 2H), 5.15 (s, 1H), 5.05 (s, 1H), 3.90 (q, 2H), 3.55 (s, 1H), 3.15 (s, broad, 2H), 2.90-2.70 (m, 2H), 2.45 (s, 3H). MS (ES+) 447.06.

### Example 17G

### COMPOUND 4-7

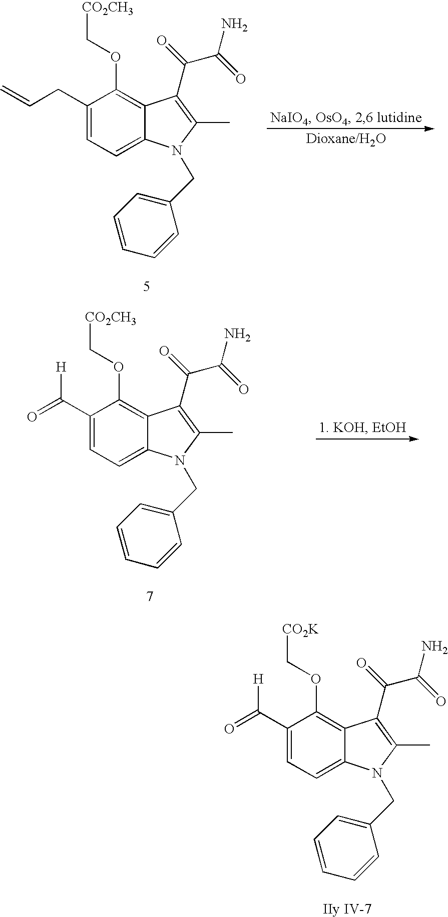

(3-Aminooxalyl-1-benzyl-5-formyl-2-methyl-1H-indol-4-yloxy)-acetic acid methyl ester 7: (5-Allyl-3-aminooxalyl-1-benzyl-2-methyl-1H-indol-4-yloxy)-acetic acid methyl ester 5 (0.32 g, 0.762 mmole) was dissolved in dioxane:H₂O 3:1 (8 mL). To the mixture 2, 6 lutidine (0.17 mL, 1.437 mmole), sodium periodate (0.67 g, 3.128 mmole) and osmium tetroxide (5 grains) were added. The mixture was stirred at room temperature for 48 h. The reaction mixture was diluted with ethyl acetate (400 mL), then washed with H₂O (3×100 mL) and brine (1×100 mL). The organic layer was separated, dried with magnesium sulfate and concentrated. The residue was purified by column chromatography 3:1 EtOAc:Hex 1% Et₃N to afford 7 (0.1 g, 34%) as a yellow solid.

(3-Aminooxalyl-1-benzyl-5-formyl-2-methyl-1H-indol-4-yloxy)-acetic acid (potassium salt) IIy-IV-7: (3-Aminooxalyl-1-benzyl-5-formyl-2-methyl-1H-indol-4-yloxy)-acetic acid methyl ester 7 (0.04 g, 0.098 mmole) was dissolved in anhydrous ethanol (10 mL). To the mixture 0.5054 N potassium hydroxide solution (0.193 mL, 0.098 mmole) was added. The mixture was stirred at room temperature for 2.5 h. The reaction mixture was evaporated under high vacuum to dryness. The residue was stirred in diethyl ether for 30 minutes and collected by filtration. The solid was purified by preparative TLC (EtOAc 100%) to afford IIy-IV-7 (0.020 mg, 47%) as an orange solid.

Ref: 04-090-265.3: ¹H NMR (DMSO) δ 10.60 (s, 1H), 8.53 (s, 1H), 7.52-7.47 (m, 2H), 7.33-7.26 (m, 4H), 7.06 (m 2H), 5.53 (s, 2H), 4.10 (s, 2H), 2.46 (s, 3H). MS (ES−) 393.02, (ES+) 395.02 (H⁺), 417.00 (Na⁺), 432.96 (K⁺).

### Example 17H

### COMPOUND 4-2

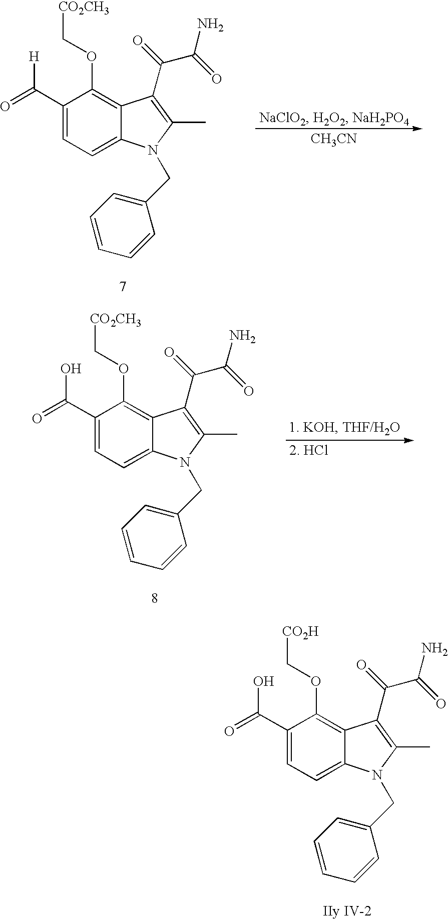

3-Aminooxalyl-1-benzyl-4-methoxycarbonylmethoxy-2-methyl-1H-indole-5 carboxylic acid 8: (3-Aminooxalyl-1-benzyl-5-formyl-2-methyl-1H-indol-4-yloxy)-acetic acid methyl ester 7 (0.05 g, 0.122 mmole) was mixed in acetonitrile (10 mL). To the mixture hydrogen peroxide 30% wt (0.012 mL, 0.1225 mmole) and 0.033M NaH₂PO₄ solution (1 mL, 0.033 mmole) were added. The mixture was cooled with an ice bath. To the mixture 0.177M NaClO₂ solution (1 mL, 0.177 mmole) was added drop-wise over 30 min. The mixture was stirred at room temperature for 24 h. To the reaction mixture sodium sulfite was added, the solution was then acidified with 2M HCl. The acetonitrile was evaporated and the mixture extracted with ethyl acetate (100 mL) and the organic layer washed with H₂O (2×100 mL) and brine (1×100 mL). The organic layer was separated and concentrated. The residue was purified by preparative TLC (3:1 EtOAc:Hex) to afford 8 (40 mg, 77%) as a yellow solid.

3-Aminooxalyl-1-benzyl-4-carboxymethoxy-2-methyl-1H-indole-5-carboxylic acid IIy-IV-2: 3-Aminooxalyl-1-benzyl-4-methoxycarbonylmethoxy-2-methyl-1H-indole-5 carboxylic acid 8 (0.035 g, 0.082 mmole) was dissolved in THF:H₂O 4:1 (10 mL). To the mixture 0.5054 N potassium hydroxide solution (0.2 mL, 0.201 mmole) was added. The mixture was stirred at room temperature for 30 min. The reaction mixture was evaporated to dryness under high vacuum. The residue was dissolved in H₂O (5 mL) and the solution acidified with 2M HCl. The resulting solid was collected by filtration washed with H₂O and diethyl ether and dried to afford IIy-IV-2 (0.011 g, 32%) as a yellow solid.

Ref: 04-090-269.1: ¹H NMR (DMSO) δ 7.71 (s, broad, 1H), 7.63 (m, 1H), 7.55 (s, broad, 1H), 7.40-7.20 (m, 4H), 7.05 (m, 2H), 5.55 (s, 2H), 4.55 (s, 2H), 2.50 (s, 3H). MS (ES−) 408.97.

### Example 17I

### COMPOUND 4-23

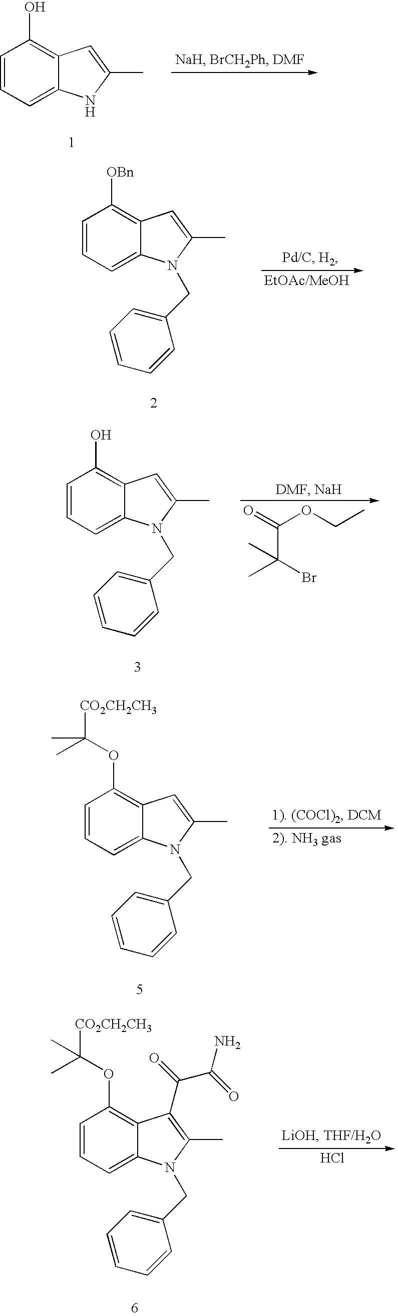

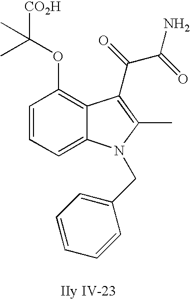

1-Benzyl-4-benzyloxy-2-methyl-1H-indole 2: 4-hydroxy-2-methyl indole 1 (50 g, 0.339 mole) was dissolved in anhydrous DMF (1 L). To the mixture sodium hydride 60% in mineral oil (27.9 g, 0.697 mole) was added. The mixture was left to stir at rt. for 1 h. To the mixture benzyl bromide (82.7 mL, 0.697 mole) was added drop-wise. The mixture was left to stir at room temperature for 18 h. The reaction was diluted with ethyl acetate (4 L) and washed with water (5×500 mL) then brine (1 L). The organic layer was separated and dried with magnesium sulphate and concentrated. The orange oily residue was purified by column chromatography (6:1 Hexane:EtOAc) to afford 86 g (72%) of 2 as an yellow oil.

1-Benzyl-2-methyl-1H-indol-4-ol 3: 1-Benzyl-4-benzyloxy-2-methyl-1H-indole 2 (86 g, 0.263 mole) was dissolved with ethyl acetate (1.5 L) and methanol (300 mL). To the mixture 10% Pd/C wet (18 g) was added to the solution. The reaction was then subjected to H₂ gas passed through a mercury bubbler at room temperature and 1 atm. The mixture was left to stir for 6 h. The reaction mixture was filtered through Celite and concentrated. The residue was purified by column chromatography (3:1 Hexane:EtOAc) to afford 3 (30 g, 49%) as a cream solid.

2-(1-Benzyl-2-methyl-1H-indol-4-yloxy)-2-methyl-propionic acid ethyl ester 5: 1-Benzyl-2-methyl-1H-indol-4-ol 3 (0.3 g 1.26 mmole) was dissolved in anhydrous dimethylformamide (50 mL). To the solution sodium hydride 60% in mineral oil (66 mg 1.65 mmole) was added. The mixture was stirred at room temperature for 1 h. To the mixture ethyl-2-bromoisobutyrate (0.243 mL, 1.65 mmole) was added. The mixture was stirred at room temperature for 18 h. The reaction was diluted with ethyl acetate (500 mL) and washed with H₂O (5×100 mL) and brine (1×100 mL). The organic layer was separated, dried with magnesium sulfate and concentrated. The residue was purified by column chromatography (6:1 Hexane:EtOAc) to afford 5 (0.07 g, 16%) as an yellow oil.

2-(3-Aminooxalyl-1-benzyl-2-methyl-1H-indol-yloxy)-2-methyl-propionic acid ethyl ester 6: To a solution of oxalyl chloride (0.02 mL, 0.218 mmole) in anhydrous dichloromethane (25 mL) a solution 2-(1-Benzyl-2-methyl-1H-indol-4-yloxy)-2-methyl-propionic acid ethyl ester 5 (0.07 g, 0.199 mmole) in anhydrous dichloromethane (25 mL) was added drop-wise. The mixture was left to stir at room temperature for 2 h. NH₃ gas was then bubbled through the solution for 30 minutes. The mixture was left to stir for 1.5 h at room temperature. The dichloromethane was evaporated and the residue dissolved in ethyl acetate (200 mL) and washed with H₂O (3×200 mL) and brine (1×300 mL). The organic layer was separated, dried with magnesium sulfate and concentrated to afford 6 (0.1 g, >100%) as a yellow solid (contained inorganic salt). The material was used in next step without further purification.

2-(3-Aminooxalyl-1-benzyl-2-methyl-1H-indol-4-yloxy)-2-methyl-propionic acid Idly-IV-23: 2-(3-Aminooxalyl-1-benzyl-2-methyl-1H-indol-yloxy)-2-methyl-propionic acid ethyl ester 6 (0.12 g, 0.284 mmole) was dissolved in THF:H₂O 4:1 (10 mL). To the mixture lithium hydroxide monohydrate (0.042 g, 1.00 mmole) was added. The mixture was left to stir at room temperature for 18 h. Reaction was heated at 50° C. for 18 h. The mixture was acidified to pH 3 with 2M HCl. The resulting precipitate was collected by filtration and washed with water to afford IIy-IV-23 (0.030 g, 27%) as a yellow solid.

Ref: 04-090259.2: ¹H NMR (DMSO) δ 7.85 (s, 1H), 7.55 (s, 1H), 7.35-6.95 (m, 7H), 6.32 (d, 1H), 5.48 (s, 2H), 2.25 (s, 3H), 1.55 (s, 6H). MS (ES+) 395.07

### Example 17J

### COMPOUND 4-32

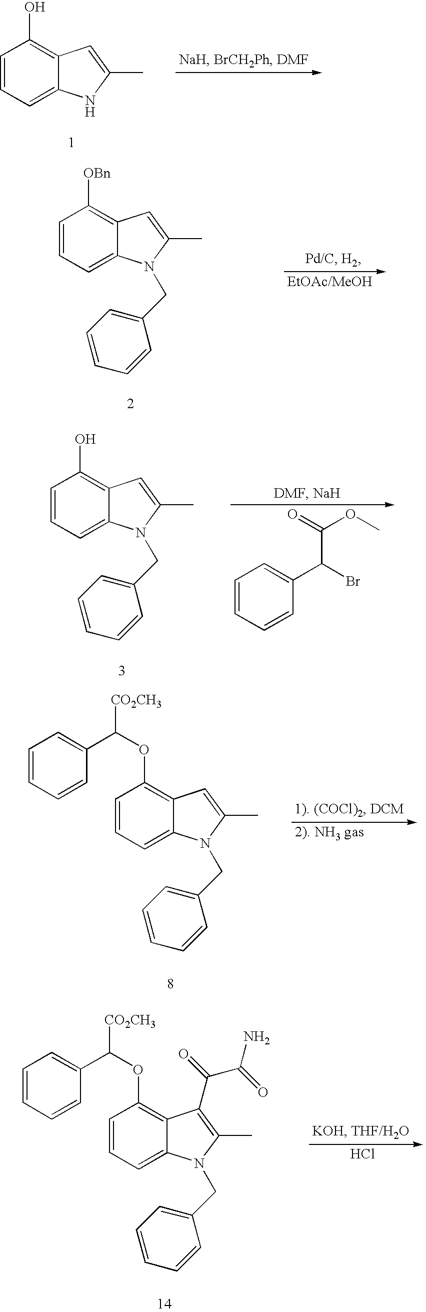 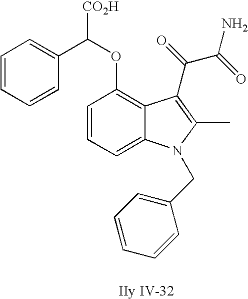 1-Benzyl-4-benzyloxy-2-methyl-1H-indole 2: 4-hydroxy-2-methyl indole 1 (50 g, 0.339 mole) was dissolved in anhydrous DMF (1 L). To the mixture sodium hydride 60% in mineral oil (27.9 g, 0.697 mole) was added. The mixture was left to stir at rt. for 1 h. To the mixture benzyl bromide (82.7 mL, 0.697 mole) was added drop-wise. The mixture was left to stir at room temperature for 18 h. The reaction was diluted with ethyl acetate (4 L) and washed with water (5×500 mL) then brine (1 L). The organic layer was separated and dried with magnesium sulphate and concentrated. The orange oily residue was purified by column chromatography (6:1 Hexane:EtOAc) to afford 86 g (72%) of 2 as an yellow oil.

1-Benzyl-2-methyl-1H-indol-4-ol 3: 1-Benzyl-4-benzyloxy-2-methyl-1H-indole 2 (86 g, 0.263 mole) was dissolved with ethyl acetate (1.5 L) and methanol (300 mL). To the mixture 10% Pd/C wet (18 g) was added to the solution. The reaction was then subjected to H₂ gas passed through a mercury bubbler at room temperature and 1 atm. The mixture was left to stir for 6 h. The reaction mixture was filtered through Celite and concentrated. The residue was purified by column chromatography (3:1 Hexane:EtOAc) to afford 3 (30 g, 49%) as a cream solid.

(1-Benzyl-2-methyl-1H-indol-4-yloxy)-phenyl-acetic acid methyl ester 8: 1-Benzyl-2-methyl-1H-indol-4-ol 3 (0.3 g 1.26 mmole) was dissolved in anhydrous dimethylformamide (20 mL). To the solution sodium hydride 60% in mineral oil (66 mg 1.65 mmole) was added. The mixture was stirred at room temperature for 1 h. To the mixture bromo-phenyl-acetic acid methyl ester (0.24 mL, 1.512 mmole) was added. The mixture was stirred at room temperature for 18 h. The reaction was diluted with ethyl acetate (300 mL) and washed with H₂O (4×100 mL) and brine (1×100 mL). The organic layer was separated, dried with magnesium sulfate and concentrated. The residue was purified by column chromatography (10:1 Hexane:EtOAc) to afford 8 (0.3 g, 62%) as a white solid.

(3-Aminooxalyl-1-benzyl-2-methyl-1H-indol-4-yloxy)-2-phenyl-acetic acid methyl ester 14: (1-Benzyl-2-methyl-1H-indol-4-yloxy)-phenyl-acetic acid methyl ester 8 (0.15 g, 0.389 mmole) was dissolved in anhydrous dichloromethane (50 mL). To the solution oxalyl chloride (0.04 mL, 0.428 mmole) was added. The mixture was left to stir at room temperature for 2 h. NH₃ gas was then bubbled through the solution for 30 minutes. The mixture was left to stir at room temperature for 1 h. The dichloromethane was evaporated and the residue was dissolved in ethyl acetate (200 mL) and washed with H₂O (3×200 mL) and brine (1×300 mL). The organic layer was separated, dried with magnesium sulfate and concentrated to afford 14 (0.15 g, 85%) as a yellow solid.

(3-Aminooxalyl-1-benzyl-2-methyl-1H-indol-4-yloxy)-phenyl-acetic acid IIy-IV-32: (3-Aminooxalyl-1-benzyl-2-methyl-1H-indol-4-yloxy)-2-phenyl-acetic acid methyl ester 14 (0.15 g, 0.33 mmole) was dissolved in THF:H₂O 4:1 (10 mL). To the mixture 0.5054 N potassium hydroxide solution (0.48 mL, 0.495 mmole) was added. The mixture was left to stir at room temperature for 18 h. The reaction mixture was evaporated to dryness. The residue was dissolved in H₂O (5 mL) and acidified to pH 4 with 2M HCl. The resulting precipitate was collected by filtration washed with H₂O and dried to afford IIy-IV-32 (0.08 g, 55%) as a yellow solid.

Ref: 04-090-281.1: ¹H NMR (DMSO) δ 12.90 (s, broad, 1H), 7.90 (s, broad, 1H), 7.65 (d, 2H), 7.50-7.00 (m, 11H), 6.60 (d, 1H), 6.85 (s, 1H), 5.50 (s, 2H), 2.45 (s, 3H). MS (ES+) 443.02

### Example 17K

### COMPOUND 3-20

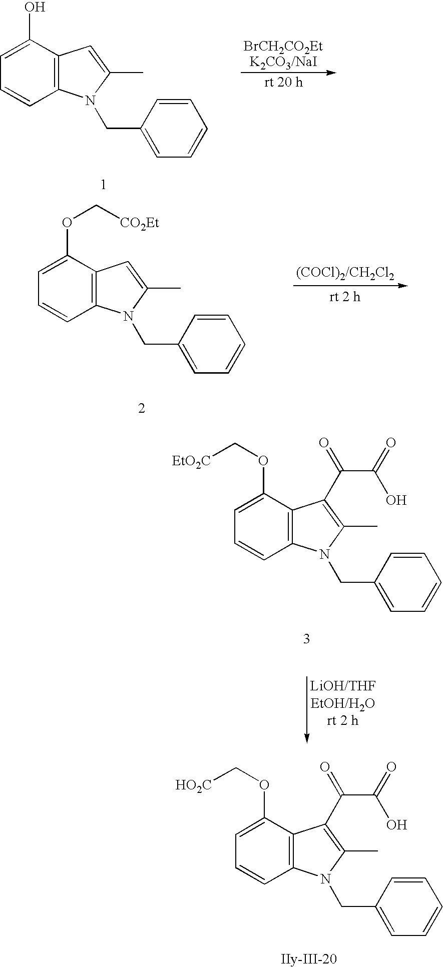

(1-Benzyl-2-methyl-1H-indol-4-yloxy)-acetic acid ethyl ester, 2. To a stirred suspension of K₂CO₃ (11.7 g, 84.7 mmol), NaI (0.633 g, 4.22 mmol) and 1-benzyl-2-methyl-1H-indol-4-ol 1 (10.0 g, 42.2 mmol) in dry DMF (100 mL), ethyl bromoacetate (5.10 mL, 46.0 mmol) was added dropwise and the reaction mixture was stirred for 20 h, then water (150 mL) was added. The mixture was extracted with EtOAc (3×150 mL). The combined organic extracts were washed with water (100 mL), brine (100 mL), dried over Na₂SO₄ and evaporated. Flash chromatography of the residue over silica gel, using 10% EtOAc in hexanes to 25% EtOAc in hexanes) gave product 2 as a pale yellow solid.

Yield: 10.3 g (76%).

(1-benzyl-4-ethoxycarbonylmethoxy-2-methyl-1H-indol-3-yl)-oxo-acetic acid 3. To a stirred solution of (1-benzyl-2-methyl-1H-indol-4-yloxy)-acetic acid ethyl ester 2 (100 mg, 0.310 mmol) in CH₂Cl₂ (4 mL), (COCl)₂ (40 μL, 0.46 mmol) was added dropwise, and then the reaction mixture was stirred at room temperature for 2 h, evaporated to give crude product 3 as a white solid, which was used without further purification for the next step.

Yield: 120 mg (100%).

(1-benzyl-4-carboxymethoxy-2-methyl-1H-indol-3-yl)-oxo-acetic acid (IIy-III-20). To a stirred solution of (1-benzyl-4-ethoxycarbonylmethoxy-2-methyl-1H-indol-3-yl)-oxo-acetic acid 3 (120 mg, 0.310 mmol) in THF/H₂O (8 mL/8 mL), LiOH (37 mg, 1.6 mmol) was added. The reaction mixture was stirred at room temperature for 2 h, evaporated and then acidified (pH=4) with 1 N HCl to form a white precipitate, which was filtered off, washed with water and dried in vacuum to afford product IIy-III-20 as a pale yellow solid. Yield: 96 mg (84%).

¹H NMR: 05-013-261 diacids (DMSO-d₆, 400 MHz) δ 2.58 (s, 3H), 4.66 (s, 2H), 5.54 (s, 2H), 6.58 (d, 1H), 7.02-7.38 (m, 7H) (COOH not shown).

MS: 366.04 (M−H).

### Example 17L

### COMPOUND 3-22

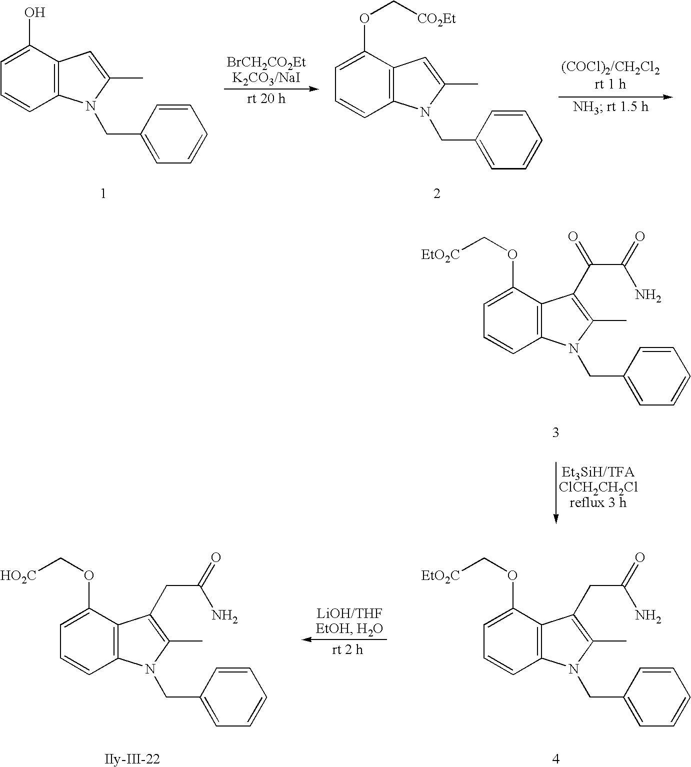

(1-Benzyl-2-methyl-1H-indol-4-yloxy)-acetic acid ethyl ester 2. To a stirred suspension of K₂CO₃ (11.7 g, 84.7 mmol), NaI (0.633 g, 4.22 mmol) and 1-benzyl-2-methyl-1H-indol-4-ol 1 (10.0 g, 42.2 mmol) in dry DMF (100 mL), ethyl bromoacetate (5.10 mL, 46.0 mmol) was added dropwise and the reaction mixture was stirred for 20 h, then water (150 mL) was added. The mixture was extracted with EtOAc (3×150 mL). The combined organic extracts were washed with water (100 mL), brine (100 mL), dried over Na₂SO₄ and evaporated. Flash chromatography of the residue over silica gel, using 10% EtOAc in hexanes to 25% EtOAc in hexanes) gave product 2 as a pale yellow solid. Yield: 10.3 g (76%).

(3-Aminooxalyl-1-benzyl-2-methyl-1H-indol-4-yloxy)-acetic acid ethyl ester 3. To a stirred solution of (1-benzyl-2-methyl-1H-indol-4-yloxy)-acetic acid ethyl ester 2 (400 mg, 1.24 mmol) in CH₂Cl₂ (10 mL), (COCl)₂ (0.14 mL, 1.6 mmol) was added dropwise, and then the reaction mixture was stirred at room temperature for 1 h, treated with NH₃ (g) for 30 min and stirred for another 1 h. The obtained mixture was diluted with EtOAc (100 mL), washed with water (50 mL), brine (50 mL), dried over Na₂SO₄ and evaporated to give crude product 3 as a yellow solid, which was used without further purification for the next step. Yield: 465 mg (95%).

(1-Benzyl-3-carbamoylmethyl-2-methyl-1H-indol-4-yloxy)-acetic acid ethyl ester 4. To a stirred solution of (3-aminooxalyl-1-benzyl-2-methyl-1H-indol-4-yloxy)-acetic acid ethyl ester 3 (85 mg, 0.22 mmol) in ClCH₂CH₂Cl (7 mL), Et₃SiH (0.5 mL) and CF₃CO₂H (0.5 mL) were added. The mixture was heated to 85° C. and stirring was continued for 3 h. The reaction mixture was cooled to room temperature and evaporated. The obtained residue was diluted with EtOAc (50 mL), washed with cold saturated NaHCO₃ (20 mL), brine (20 mL), dried over Na₂SO₄ and evaporated to give crude product 4 as a pale yellow solid, which was used without further purification for the next step. Yield: 83 mg (100%).

(1-Benzyl-3-carbamoylmethyl-2-methyl-1H-indol-4-yloxy)-acetic acid (IIy-III-22). To a stirred solution of (1-benzyl-3-carbamoylmethyl-2-methyl-1H-indol-4-yloxy)-acetic acid ethyl ester 4 (82 mg, 0.22 mmol) in THF/EtOH/H₂O (3 mL/3 mL/3 mL), LiOH (53 mg, 2.2 mmol) was added. The reaction mixture was stirred at room temperature for 2 h, evaporated and then acidified (pH=4) with 1 N HCl to form a precipitate, which was filtered off, washed with water and dried in vacuum to afford product IIy-III-22 as a pale pink solid. Yield: 55 mg (71%).

¹H NMR: 05-013-275 acid (DMSO-d₆, 400 MHz) δ 2.26 (s, 3H), 3.65(s, 2H), 4.75 (s, 2H), 5.38 (s, 2H), 6.44 (d, 1H), 6.78 (s, 1H), 6.88-7.03 (m, 4H), 7.06 (s, 1H), 7.18-7.32 (m, 3H), 13.2 (s, 1H).

MS: 352.99 (M+H).

### Example 17M

### COMPOUND 3-23

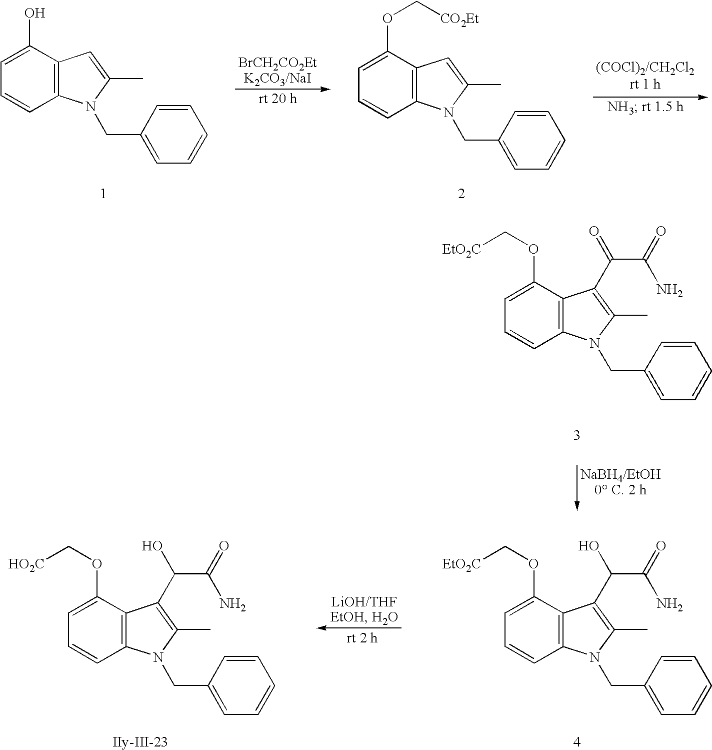

(1-Benzyl-2-methyl-1H-indol-4-yloxy)-acetic acid ethyl ester 2. To a stirred suspension of K₂CO₃ (11.7 g, 84.7 mmol), NaI (0.633 g, 4.22 mmol) and 1-benzyl-2-methyl-1H-indol-4-ol 1 (10.0 g, 42.2 mmol) in dry DMF (100 mL), ethyl bromoacetate (5.10 mL, 46.0 mmol) was added dropwise and the reaction mixture was stirred for 20 h, then water (150 mL) was added. The mixture was extracted with EtOAc (3×150 mL). The combined organic extracts were washed with water (100 mL), brine (100 mL), dried over Na₂SO₄ and evaporated. Flash chromatography of the residue over silica gel, using 10% EtOAc in hexanes to 25% EtOAc in hexanes) gave product 2 as a pale yellow solid. Yield: 10.3 g (76%).

(3-Aminooxalyl-1-benzyl-2-methyl-1H-indol-4-yloxy)-acetic acid ethyl ester 3. To a stirred solution of (1-benzyl-2-methyl-1H-indol-4-yloxy)-acetic acid ethyl ester 2 (400 mg, 1.24 mmol) in CH₂Cl₂ (10 mL), (COCl)₂ (0.14 mL, 1.6 mmol) was added dropwise, and then the reaction mixture was stirred at room temperature for 1 h, treated with NH₃ for 30 min and stirred for another 1 h. The precipitated mixture was diluted with EtOAc (100 mL), washed with water (50 mL), brine (50 mL), dried over Na₂SO₄ and evaporated to give crude product 3 as a yellow solid, which was used without further purification for the next step. Yield: 465 mg (95%).

[1-Benzyl-3-(carbamoyl-hydroxy-methyl)-2-methyl-1H-indol-4-yloxy]-acetic acid ethyl ester 4. To a stirred solution of (3-aminooxalyl-1-benzyl-2-methyl-1H-indol-4-yloxy)-acetic acid ethyl ester 3 (50 mg, 0.13 mmol) in EtOH/CH₂Cl₂ (3 mL/3 mL), NaBH₄ (6.6 mg, 0.17 mmol) was added. The mixture was stirred at 0° C. for 2 h and evaporated. The residue was diluted with EtOAc (20 mL), washed with water (10 mL), brine (10 mL), dried over Na₂SO₄ and evaporated. Flash chromatography of the residue over silica gel, using 15% EtOAc in hexanes to 40% EtOAc in hexanes, gave product 4 as a white solid. Yield: 40 mg (80%).

[1-Benzyl-3-(carbamoyl-hydroxy-methyl)-2-methyl-1H-indol-4-yloxy]-acetic acid (IIy-III-23). To a stirred solution of [1-benzyl-3-(carbamoyl-hydroxy-methyl)-2-methyl-1H-indol-4-yloxy]-acetic acid ethyl ester 4 (40 mg, 0.10 mmol) in THF/EtOH/H₂O (3 mL/3 mL/3 mL), LiOH (24 mg, 1.0 mmol) was added. The reaction mixture was stirred at room temperature for 2 h, evaporated and then acidified (pH=4) with 1 N HCl to form a white precipitate, which was filtered off, washed with water and dried in vacuum to afford product IIy-III-23 as an off-white solid. Yield: 32 mg (87%)

¹H NMR: 05-013-295 (DMSO-d₆, 400 MHz) δ 2.32 (s, 3H), 4.56 (AB q, 2H), 5.36 (s, 2H), 5.43 (s, 1H), 6.48 (d, 1H), 6.88-7.02 (m, 4H), 7.05 (s, 1H), 7.18-7.33 (m, 3H), 7.76 (s, 1H) (COOH not shown).

MS: 366.98 (M−H)

### Example 17N

### COMPOUND 4-21

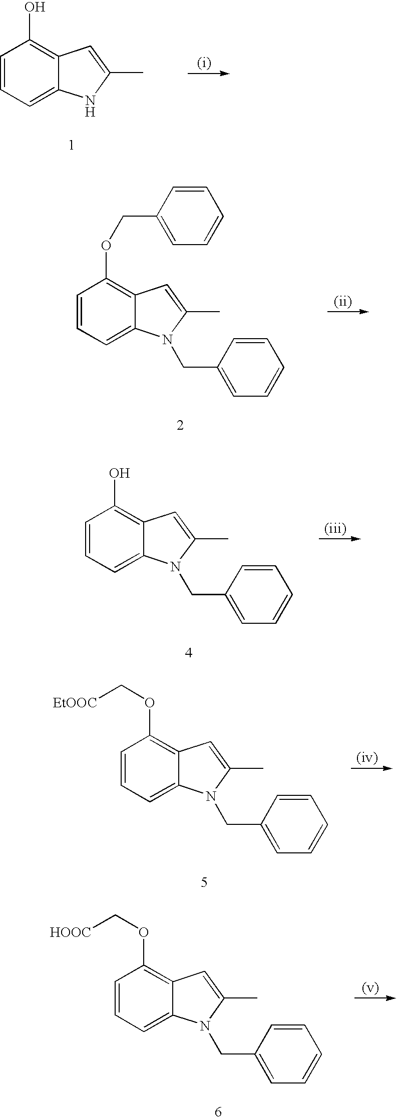

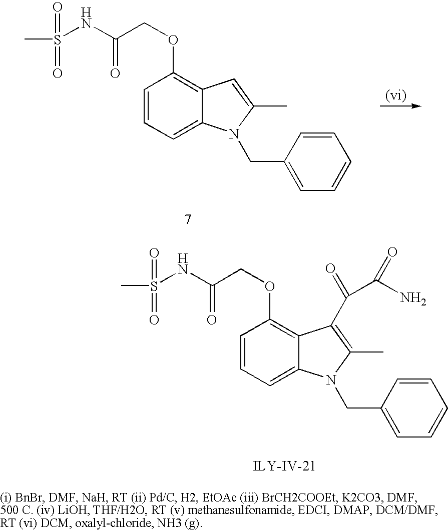

1-Benzyl-2-methyl-4-methanesulfonamidoylmethyloxy-1H-indol-3-glyoxylamide (ILY-IV-21) To a solution of 2-(1-benzyl-2-methyl-1H-indol-4-yloxy)acetic acid (6, 670 mg, 2.3 mmol) in dichloromethane/dimethylformamide mixture (4:1, 10 mL) was added 4-dimethylaminopyridine (415 mg 3.4 mmol), methanesulfonamide (431 mg, 4.5 mmol) and 1-(3-dimethylaminopropyl)-3-ethylcarbodiimide hydrochloride (433 mg, 2.3 mmol) and the reaction mixture was stirred at room temperature. After 24 h the reaction mixture was diluted with dichloromethane and washed twice with 1N HCl and brine. The organic layer was dried with Na₂SO₄ and evaporated in vacuum. The residue was chromatographed on silica gel (CHCl₃ to 4% MeOH in CHCl₃) to give 1-benzyl-2-methyl-4-methanesulfonamidoylmethyloxy-1H-indol (7, 485 mg, 57%).

A solution of 1-benzyl-2-methyl-4-methanesulfonamidoylmethyloxy-1H-indol (7, 190 mg, 0.5 mmol) in 20 mL dichloromethane was stirred at 0° C. and solution of oxalylchloride (78 mg, 0.6 mmol) in 4 mL of dichloromethane was added drop wise. The solution was allowed to stir for 1 h at room temperature. Ammonia was bubbled through the solution for 30 minutes followed by diluting the reaction mixture with diethyl ether. The solid thus obtained filtered to give pale yellow solid which after chromatography on silica gel (6% MeOH in CHCl₃) gave pure ILY-IV-21 in 75% yield (170 mg).

¹H NMR (400 MHz, DMSO-d₆) δ 7.85 (brs, 1H), 7.52 (brs, 1H), 7.22-7.39 (m, 3H), 7.02-7.19 (m, 4H), 6.44 (d, 1H), 5.43 (s, 2H), 4.40(s, 2H), 2.96(s, 3H), 2.42(s, 3H) ppm.

MS (ESI) m/z 443.95 (M+1).

### Example 17O

### COMPOUND 4-26

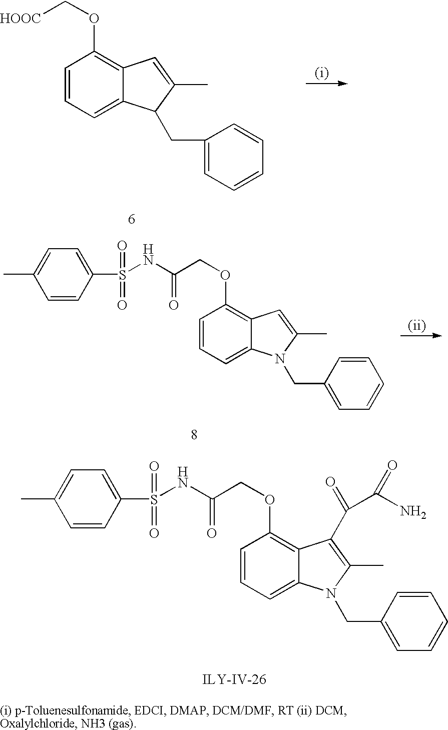

1-Benzyl-2-methyl-4-p-toluenesulfonamidoylmethyloxy-1H-indol-3-glyoxylamide (ILY-IV-26) To a solution of 2-(1-benzyl-2-methyl-1H-indol-4-yloxy)acetic acid (6, 200 mg, 0.8 mmol) in dichloromethane/dimethylformamide (4:1, 8 mL) was added 4-dimethylaminopyridine (146 mg 1.2 mmol), p-toluenesulfonamide (273 mg, 1.6 mmol) and 1-(3-dimethylaminopropyl)-3-ethylcarbodiimide hydrochloride (153 mg, 0.8 mmol), and reaction mixture was allowed to stir at room temperature. After 24 h the reaction was diluted with dichloromethane and washed twice with 1N HCl and brine. The organic layer was dried with Na₂SO₄ and evaporated in vacuum. The residue was chromatographed on silica gel (CHCl₃ to 2% MeOH in CHCl₃) to give the compound 8 (170 mg, 56%).

A solution of 1-benzyl-2-methyl-4-para-toluenesulfonamidoylmethyloxy-1H-indol (8, 167 mg, 0.4 mmol) in 20 mL dichloromethane was stirred at 0° C. and solution of oxalylchloride (56 mg, 0.5 mmol) in 4 mL of dichloromethane was added drop wise and solution allowed to stir for 1 h at room temperature. Ammonia was bubbled through the solution for 30 minute after this reaction was diluted with diethyl ether and solid was filtered to give pale yellow solid which after chromatography on silica gel (5% MeOH in CHCl₃) gave pure ILY-IV-26 in 69% yield (133 mg).

¹H NMR (400 MHz, DMSO-d₆) δ 11.91(brs, 1H), 8.00 (brs, 1H), 7.59-7.64 (m, 3H), 7.24-7.36 (m, 5H), 7.20 (d, 1H), 7.15 (d, 2H), 6.98 (t, 1H), 6.23 (d, 1H), 5.24 (s, 2H), 4.61 (s, 2H), 2.46 (s, 3H), 2.32 (s, 3H) ppm.

MS (ESI) m/z 520.01 (M+1).

### Example 17P

### COMPOUND 4-30

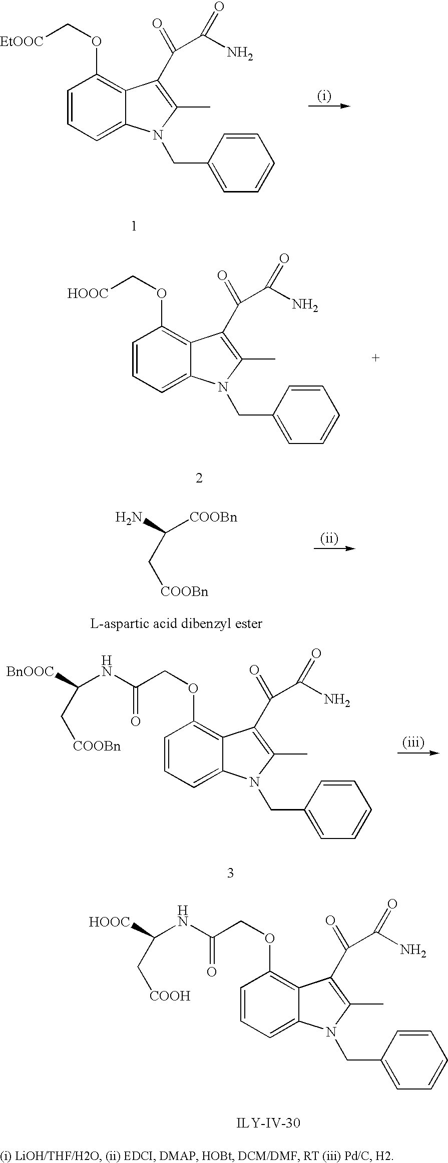

1-Benzyl-2-methyl-4-asparticaciddibenzylesteramidoylmethyloxy-1H-indol-3-glyoxylamide, 3 To a solution of 2-[[3-(2-amino-1,2-dioxoethyl-2-methyl-1-(benzyl)-1H-indol-4-yl]oxy]acetic acid 2 (549 mg, 1.5 mmol) in dichloromethane/dimethylformamide (5:1) was added aspartic acid dibenzyl ester (313 mg 1.5 mmol), 4-dimethylaminopyridine (18 mg 0.15 mmol), 1-hydroxybenzotriazole (202 mg, 1.5 mmol) and 1-(3-dimethylaminopropyl)-3-ethylcarbodiimide hydrochloride (286 mg, 1.5 mmol), respectively and reaction mixture allowed to stir at room temperature. After 6 hrs the reaction was diluted with dichloromethane and washed twice with 1N HCl and brine. The organic layer was dried with Na₂SO₄ and evaporated in vacuum. The residue was chromatographed on silica gel EtOAc:Hexanes 40 to 60% to give the titled compound 3 (620 mg, 62%).

1-Benzyl-2-methyl-4-asparticacidamidoylmethyloxy-1H-indol-3-glyoxylamide (ILY-IV-30) A solution of 3 (170 mg, 0.25 mmol) in ethanol 10 mL was stirred in hydrogen atmosphere using a balloon in the presence of Pd/C 50 mg. After stirring for 18 h the catalyst was filtered through celite and solvent was evaporated to give the target compound ILY-IV-30 103 mg (84% yield).

¹H NMR (400 MHz, DMSO-d₆) δ 8.45 (d, 1H), 7.96 (brs, 1H), 7.42 (brs, 1H), 7.20-7.38 (m, 4H), 7.00-7.18 (m, 3H), 6.61 (d, 1H), 5.45 (s, 2H), 4.61-4.63 (m, 1H), 4.48 (s, 2H), 2.61-2.67 (m, 1H), 2.78-2.81 (m, 1H), 2.45 (s, 3H) ppm.

MS (ESI) m/z 481.95 (M+1).

### Example 17Q

### COMPOUND 4-31

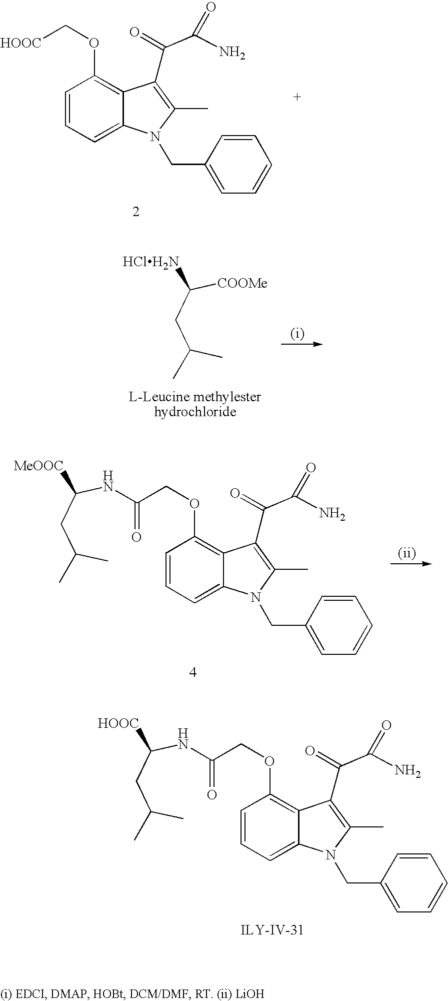

1-Benzyl-2-methyl-4-leucinemethylesteramidoylmethyloxy-1H-indol-3-glyoxylamide 4 To a solution of 2-[[3-(2-amino-1,2dioxoethyl-2-methyl-1-(phenylmethyl)-1H-indol-4-yl]oxy]acetic acid 2 (270 mg, 0.7 mmol) in dichloromethane/dimethylformamide mixture (5:1) was added leucine methyl ester hydrochloride (134 mg 0.7 mmol), 4-dimethylaminopyridine (108 mg 0.84 mmol), 1-hydroxybenzotriazole (99 mg, 0.7 mmol) and 1-(3-dimethylaminopropyl)-3-ethylcarbodiimide hydrochloride (139 mg, 0.7 mmol). After 6 h the reaction was diluted with dichloromethane and washed twice with 1N HCl and brine. The organic layer was dried over Na₂SO₄ and evaporated in vacuum. The residue was chromatographed on silica gel using EtOAc:Hexanes gradient 50 to 70% of EtOAc in hexanes to give 4 (217 mg, 60%).

¹H NMR, mass and HPLC data: 03-038-105.

1-Benzyl-2-methyl-4-leucinamidoylmethyloxy-1H-indol-3-glyoxylamide (ILY-IV-31) A solution of 4 (200 mg, 0.4 mmol) in THF/H₂O 4:1 was stirred with 2.2 equivalent of LiOH for 30 minutes at room temperature. After completion of reaction, THF was evaporated and resulting residue was neutralized with dilute HCl at 0° C. The yellow solid thus obtained was filtered and washed with water and then hexanes to give 110 mg (56% yield) of ILY-IV-31.

¹H NMR (400 MHz, DMSO-d₆) δ 12.59 (brs, 1H), 8.42 (d, 1H), 8.07 (brs, 1H), 7.61 (brs, 1H), 7.18-7.33 (m, 4H), 7.03-7.13 (m, 3H), 6.64 (d, 1H), 5.45 (s, 2H), 4.59 (s, 2H), 4.21 (t, 1H), 2.54 (s, 3H), 1.61-1.69 (m, 1H), 1.47-1.50 (m, 2H), 0.78 (d, 3H), 0.74 (d, 3H) ppm.

MS (ESI) m/z 480.00 (M+1).

### Example 17R and 17S

### COMPOUND 4-29 AND 4-34

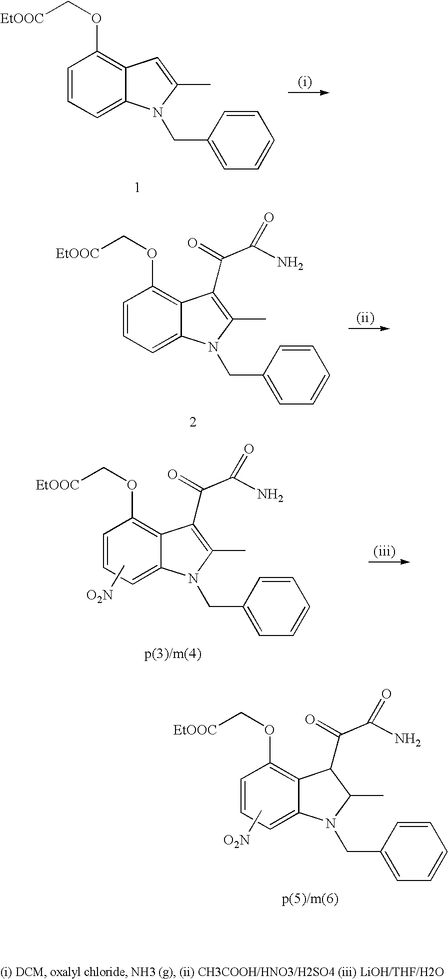

2-[[3-(2-amino-1,2-dioxoethyl-2-methyl-7 & 5-nitro-1-(benzyl)-1H-indol-4-yl]oxy]ethyl acetate(3 & 4) 2-[[3-(2-amino-1,2-dioxoethyl-2-methyl-1-(benzyl)-1H-indol-4-yl]oxy]ethyl acetate 2 (400 mg, 1.0 mmol) was suspended in 20 mL of glacial acetic acid and suspension was cooled in ice-water mixture. The solution of 90% fuming nitric acid (72 mg, 1.0 mmol) in 2 mL of acetic acid was added drop wise followed by the addition of 10 mg of H₂SO₄. After stirring for 45 min the reaction was poured into crushed ice and yellow solid was filtered and repeatedly washed with cold water. The crude product was purified on silica gel using the EtOAc:hexanes gradient (20% to 60% EtOAc in hexanes) give the mixture of para 3 (112 mg) and meta 4 (89 mg) isomers in 25% and 20% isolated yields.

2-[[3-(2-amino-1,2-dioxoethyl-2-methyl-7-nitro-1-(benzyl)-1H-indol-4-yl]oxy]acetic acid, 5 (ILY-IV-29) A solution of 3 (55 mg, 0.12 mmol) in THF/H₂O 4:1 was stirred with 2.2 equivalent of LiOH and solution was allowed to stir for 30 minutes at room temperature. After completion of reaction THF was evaporated and resulting residue was neutralized with dilute HCl at 0° C. The yellow solid thus obtained was filtered and washed with water and then diethyl ether to give 34 mg (67% yield) of ILY-IV-29.

¹H NMR (400 MHz, DMSO-d₆) δ 8.16 (brs, 1H), 7.71(d, 1H), 7.56 (brs, 1H), 7.22-7.35 (m, 3H), 6.68 (m, 2H), 6.61 (d, 1H), 5.42 (s, 2H), 4.48 (s, 2H), 2.42(s, 3H) ppm.

MS (ESI) m/z 411.97 (M+1).

2-[[3-(2-amino-1,2dioxoethyl-2-methyl-5-nitro-1-(benzyl)-1H-indol-4-yl]oxy]acetic acid, 6 (ILY-IV-34) A solution of 4 (80 mg, 0.18 mmol) in THF/H₂O 4:1 was stirred with 2.2 equivalents of LiOH and solution was allowed to stir for 30 minutes at room temperature. After completion of reaction THF was evaporated and resulting residue was neutralized with dilute HCl at 0° C. The yellow solid thus obtained was filtered and washed with water and then diethyl ether to give ILY-IV-34 (50 mg, 65% yield).

¹H NMR (400 MHz, DMSO-d₆) δ 8.21 (s, 1H), 7.81(brs, 1H), 7.45 (brs, 1H), 7.29 (s, 1H), 7.25-7.28 (m, 3H), 7.20-7.23 (m, 2H), 5.62 (s, 2H), 4.67(s, 2H), 2.41 (s, 3H) ppm.

MS (ESI) m/z 412.03 (M+1).

### Examples 17T and 17U

### COMPOUNDS 4-35 AND 4-36

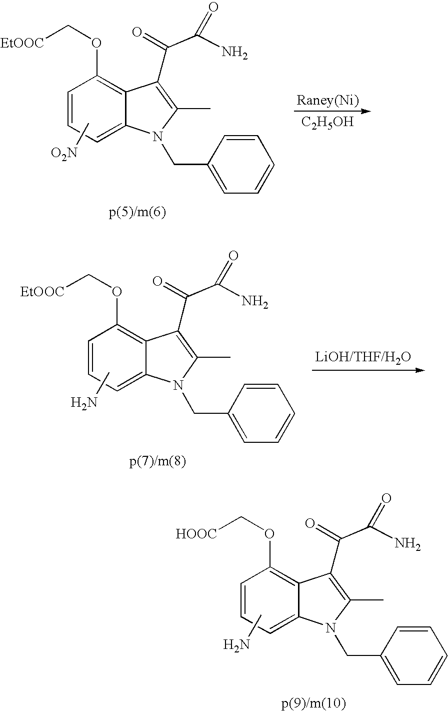

2-[[3-(2-amino-1,2-dioxoethyl-2-methyl-6 & 7-amino-1-(benzyl)-1H-indol-4-yl]oxy]ethyl acetate(7 & 8) The mixture of para and meta isomers (5 and 6, 800 mg) were dissolved in anhydrous ethanol (20 mL) and hydrogenated at 50 atm in the presence of raney (Ni) 200 mg. After stirring for 6 h the reaction was filtered through celite pad and solvent was evaporated. The mixture of amines isomers were separated by column chromatography using ethyl acetate and hexanes gradient (60 to 80% ethylacetate in hexanes)

2-[[3-(2-amino-1,2-dioxoethyl-2-methyl-7-amino-1-(benzyl)-1H-indol-4-yl]oxy]acetic acid, 9 (ILY-IV-35) A solution of 7 (60 mg) in THF/H₂O 4:1 was stirred with 2.2 equivalent of KOH and solution was allowed to stir for 30 minutes at room temperature. After completion of reaction THF was evaporated and resulting residue was neutralized with dilute HCl at 0° C. The clear solution thus obtained was evaporated and extracted with ethanol to give 4 mg (7% yield) of ILY-IV-35.

¹H NMR (400 MHz, CH₃OH-d₄) δ 7.26-7.32 (m, 3H), 7.00-7.7.02(m, 2H), 6.42 (d, 1H), 76.38 (d, 1H), 5.64 (s, 2H), 4.41 (s, 2H), 2.41(s, 3H) ppm.

MS (ESI) m/z 381.96 (M+1).

2-[[3-(2-amino-1,2dioxoethyl-2-methyl-6-amino-1-(benzyl)-1H-indol-4-yl]oxy]acetic acid, 10 (ILY-IV-36) A solution of 8 (40 mg) in THF/H₂O 4:1 was stirred with 2.2 equivalents of LiOH and solution was allowed to stir for 30 minutes at room temperature. After completion of reaction THF was evaporated and resulting residue was neutralized with dilute HCl at 0° C. The brown solid thus obtained was filtered and washed with water and then diethyl ether to give ILY-IV-36 (17 mg, 45% yield).

¹H NMR (400 MHz, DMSO-d₆) δ 7.92 (brs, 1H), 7.45(brs, 1H), 7.29-7.34 (m, 3H), 7.15-7.19 (m, 2H), 6.98 (s, 1H), 6.42 (s, 1H), 5.43(s, 2H), 4.62 (s, 2H), 2.41 (s, 3H) ppm.

MS (ESI) m/z 381.98 (M+1).

### Example 17V

### COMPOUND 4-37

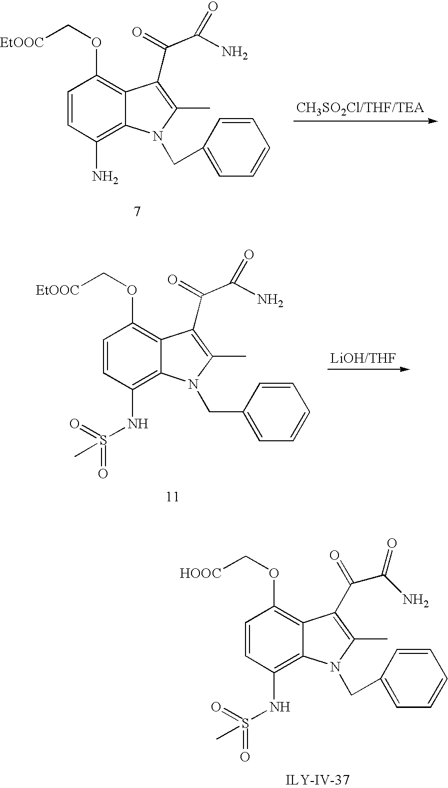

2-[[3-(2-amino-1,2-dioxoethyl-2-methyl-7-methanesulfonamido-1-(benzyl)-1H-indol-4-yl]oxy]ethylacetate, 11 A solution of 7 (100 mg, 0.24 mmol) in anhydrous dichloromethane (7 mL) was stirred with triethylamine (24 mg, 0.24 mmol) and then the solution of methanesulfonamide (13.9 mg, 0.12 mmol) in dichloromethane was added dropwise at 0° C. and reaction was allowed to proceed at room temperature. After 6 h, 0.12 mmol of methansulfomamide was added again and stirring continued. After completion of reaction solution was evaporated and resulting residue was purified by column chromatography using methanol chloroform as gradient (pure chloroform to 5% methanol in chloroform) to afford compound 11 (60 mg, 50% yield) as a pale yellow solid.

2-[[3-(2-amino-1,2-dioxoethyl-2-methyl-7-methanesulfonamido-1-(benzyl)-1H-indol-4-yl]oxy]acetic acid (ILY-IV-37) A solution of 11 (55 mg) in THF/H₂O 4:1 was stirred with 2.2 equivalents of LiOH and solution was allowed to stir for 30 minutes at room temperature. After completion of reaction THF was evaporated and resulting residue was neutralized with dilute HCl at 0° C. The brown solid thus obtained was filtered and washed with water and then diethyl ether to give ILY-IV-37 (35 mg, 68% yield).

¹H NMR (400 MHz, DMSO-d₆) δ 12.98 (brs, 1H), 9.01(brs,1H), 7.77 (brs, 1H), 7.42(brs, 1H), 7.29-7.34 (m, 3H), 6.93(d, 1H), 6.84-6.89 (m, 2H), 6.51(d, 1H), 5.91 (s, 2H), 4.60 (s, 2H), 2.92(s, 3H), 2.39 (s, 3H) ppm.

MS (ESI) m/z 459.85(M+1).

### Example 17W

### SYNTHESIS OF TERT-BUTYL 2-(3-(2-AMINO-2-OXOACETYL)-1-(8-BROMOOCTYL)-2-METHYL-1H-INDOL-4-YLOXY)ACETATE

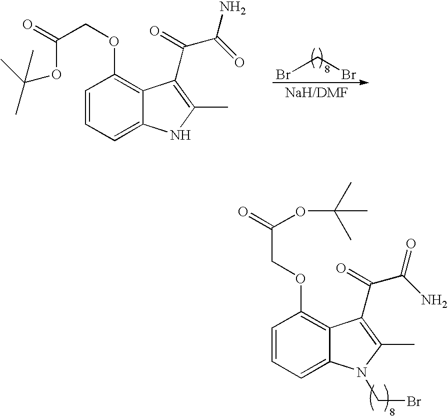

tert-Butyl 2-(3-(2-amino-2-oxoacetyl)-1-(8-bromooctyl)-2-methyl-1H-indol-4-yloxy)acetate: A solution of the starting indole (3.3 g, 10 mmol) in 10 mL of anhydrous DMF was cooled in an ice bath and dry sodium hydride (290 mg, 12 mmol, 1.2 equiv) was added. After stirring under nitrogen for 30 min at 0° C., the mixture was transferred dropwise into a solution of 1,8-dibromooctane (2.2 mL, 3.3 g, 12 mmol, 1.2 equiv) in 5 mL of anhydrous DMF also cooled in an ice bath. The resulting orange mixture was stirred under nitrogen for 4 h at 0° C., and it was then allowed to warm to RT. After an overnight stirring at RT, the reaction mixture was quenched with 15 mL of NH₄Cl and concentrated under reduced pressure. It was then diluted with 100 mL of DCM, washed with NH₄Cl (40 mL) and twice with brine (2×40 mL), dried over MgSO₄ and concentrated in vacuo to afford the crude product as an orange oil. Purification by flash-chromatography (H/EA: 3/2, 1/1 then 2/3) yielded pure bromoalkyl (2.6 g, 50%) as a yellow solid.

¹H NMR (CD₃OD, 300 MHz): δ 7.10 (dd, 1H, J=9.0, 8.1 Hz, H-6), 7.08 (dd, 1H, J=8.1, 1.5 Hz, H-5), 6.44 (dd, 1H, J=9.0, 1.5 Hz, H-7), 4.63 (s, 2H, H-10), 4.17 (t, 2H, J=7.5 Hz, H-14), 3.41 (t, 2H, J=6.9 Hz, H-15), 2.60 (s, 3H, H-9), 1.80-1.75 (m, 4H, H-16+H-17), 1.44 (s, 9H, C(CH₃)₃), 1.41-1.33 (m, 8H, CH₂).

¹³C NMR (CD₃OD, 75.5 MHz): δ 188.8 (12), 170.2 (11), 169.2 (13), 152.0 (4), 145.2 (1), 138.0 (8), 123.1 (3), 116.7 (6), 110.1 (5), 104.1 (7+2), 82.1 (C(CH₃)₃), 65.6 (10), 43.3 (14), 33.2 (15), 32.7 (17), 29.4 (16), 29.0 (CH₂), 28.5 (CH₂), 27.8 (CH₂), 27.1 (C(CH₃)₃), 26.6 (CH₂), 10.7 (9).

MS (ESI, MeOH): m/z 545.2 [M+Na]⁺ (100%, ⁷⁹Br isotope), 547.2 [M+Na]⁺ (97%, ⁸¹Br isotope).

### Example 17X

### SYNTHESIS OF TERT-BUTYL 2-(3-(2-AMINO-2-OXOACETYL)-1-(12-BROMODODECYL)-2-METHYL-1H-INDOL-4-YLOXY)ACETATE

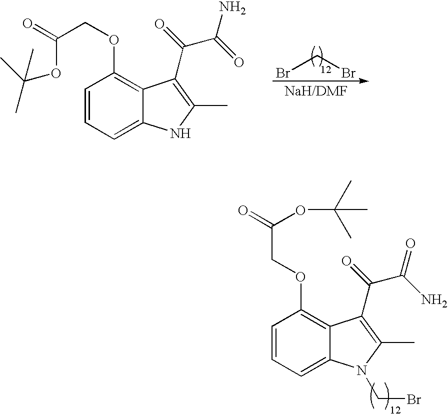

tert-Butyl 2-(3-(2-amino-2-oxoacetyl)-1-(12-bromododecyl)-2-methyl-1H-indol-4-yloxy)acetate. The starting indole intermediate (2.54 g, 7.65 mmole) in dry DMF (10 mL), at 0° C. under nitrogen, had 95% sodium hydride (0.233 g, 9.22 mmole) added. The dark mixture was stirred at 0° C. for 0.5 h and then added dropwise over 10 minutes to a solution of 1,12-dibromododecane (4.5 g, 13.71 mmole) in dry DMF (20 mL) at 0° C. The mixture was stirred at 0° C. for 5 h and at room temperature for 19 h. The reaction was cooled to 0° C., quenched with ammonium chloride solution (10 mL), and diluted with dichloromethane (100 mL). The mixture was washed with ammonium chloride solution (50 mL) and the aqueous phase extracted with dichloromethane (4×25 mL). The combined organic phase was washed with brine (100 mL), dried (Na₂SO₄), filtered and evaporated to a red/brown liquid which was further evaporated under high vacuum. The residue was a thick red/brown semi-solid, which was purified by chromatography over silica gel, using chloroform/hexanes (8:1) as the eluant, gave the product as an orange/brown semi-solid (2.00 g, 45%).

### Example 17Y

### SYNTHESIS OF ALKYL-INDOLES

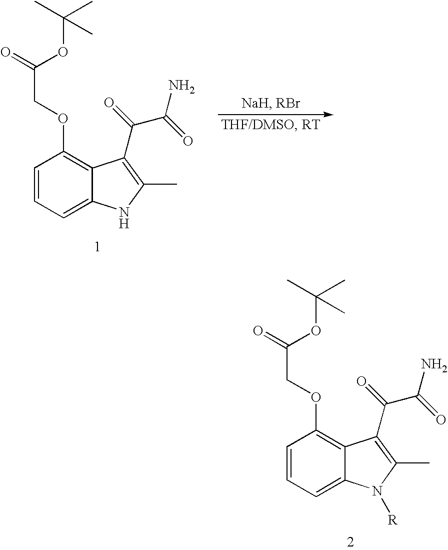

General procedure for the synthesis of alkyl-indoles (2): To Teflon sealed reaction vial containing 200 mg (0.6 mmol) of the indole 1 and NaH (26 mg, 60% dispersed in mineral oil) was added THF/DMSO (1:5, 5 mL). The reaction solution was stirred at RT for 45 minutes and then treated with the appropriate alkylating agent (neat). After stirring for 1 h, the reaction was diluted with water and extracted with ethyl acetate (3×25 mL). The combined extracts were washed with water, brine, dried over Na₂SO₄ and concentrated. Purification of the crude on silica gel (Isco CombiFlash), eluting with a gradient of 0-60% EtOAc/Heptane, to give the desired adduct.

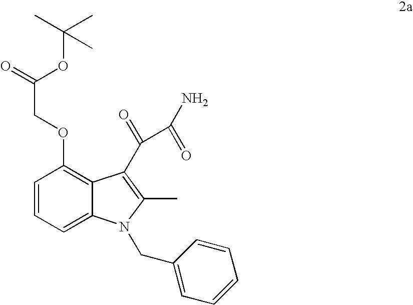

(3-Aminooxalyl-1-benzyl-2-methyl-1H-indol-4-yloxy)-acetic acid tert-butyl ester (2a): yield 190 mg, 75%; ¹H NMR (400 MHz, CDCl₃-d) δ 1.47 (s, 9H), 2.54 (s, 3H), 4.62 (s, 2H), 5.30 (s, 2H), 6.52 (d, J=7.91 Hz, 1H), 6.91 (d, J=8.25 Hz, 1H), 7.00-7.05 (m, 2H), 7.08 (t, J=8.08 Hz, 1H), 7.24-7.32 (m, 3H); ¹³C-NMR δ 11.64, 27.98, 46.94, 66.08, 82.05, 104.05, 104.30, 110.62, 116.70, 123.51, 126.03, 127.69, 128.89, 135.82, 138.12, 144.33, 151.71, 167.70, 168.16, 188.07; MS (ESI+) 422.9 (M+H).

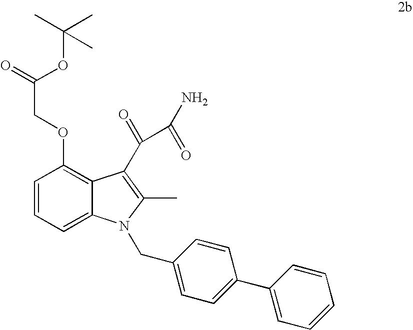

(3-Aminooxalyl-1-biphenyl-4-ylmethyl-2-methyl-1H-indol-4-yloxy)-acetic acid tert-butyl ester (2b): yield 220 mg, 73%; ¹H NMR (400 MHz, CDCl₃-d) δ 1.48 (s, 9H), 2.59 (s, 3H), 4.63 (s, 2H), 5.36 (s, 2H), 6.55 (d, J=7.81 Hz, 1H), 6.96 (d, J=7.96 Hz, 1H), 7.08-7.15 (m, 3H), 7.34 (t, J=7.32 Hz, 1H), 7.40-7.46 (m, 2H), 7.49-7.57 (m, 4H); ¹³C-NMR δ 11.72, 28.03, 46.77, 66.15, 82.08, 104.09, 104.40, 110.72, 116.78, 123.60, 126.52, 127.00, 127.42, 127.65, 128.77, 134.81, 138.17, 140.37, 140.78, 144.30, 151.78, 167.58, 168.13, 188.05; MS (ESI+) 498.9 (M+H).

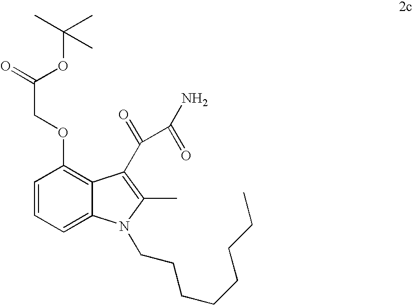

(3-Aminooxalyl-2-methyl-1-octyl-1H-indol-4-yloxy)-acetic acid tert-butyl ester (2c): This example proceeded slowly requiring an overnight reaction time to reach 80-90% completion. Yield 120 mg, 45% (55% based on recovered starting material); ¹H NMR (400 MHz, CDCl₃-d) δ 0.83-0.92 (m, 3H), 1.22-1.42 (m, 10H), 1.45 (s, 9H), 1.69-1.81 (m, 2H), 2.59 (s, 3H), 4.00-4.10 (m, 2H), 4.59 (s, 2H), 6.52 (d, J=7.91 Hz, 1H), 6.95 (d, J=8.15 Hz, 1H), 7.11 (t, J=8.05 Hz, 1H); MS (ESI+) 445.1 (M+H).

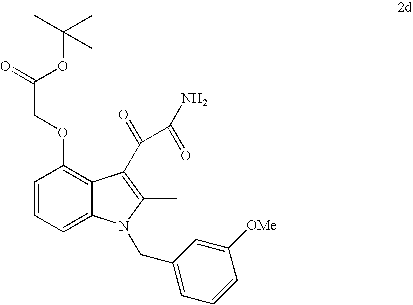

[3-Aminooxalyl-1-(3-methoxy-benzyl)-2-methyl-1H-indol-4-yloxy]-acetic acid tert-butyl ester (2d): yield 120 mg, 45% ¹H NMR (400 MHz, CDCl₃-d) δ 1.48 (s, 9H), 2.56 (s, 3H), 3.74 (s, 3H), 4.62 (s, 3H), 5.29 (s, 2H), 6.54 (d, J=7.91 Hz, 1H), 6.57-6.63 (m, 2H), 6.78-6.83 (m, 1H), 6.91-6.95 (m, 1H), 7.10 (t, J=8.08 Hz, 1H), 7.21 (t, J=8.08 Hz, 1H); MS (ESI+) 453.0 (M+H).

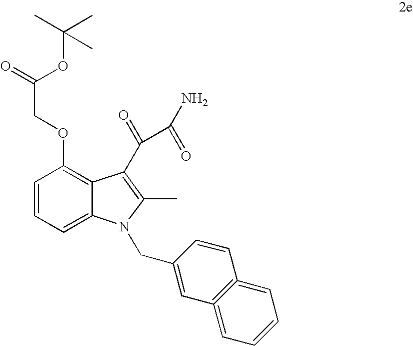

(3-Aminooxalyl-2-methyl-1-naphthalen-2-ylmethyl-1H-indol-4-yloxy)-acetic acid tert-butyl ester (2e): yield 146 mg, 51%; ¹H NMR (400 MHz, CDCl₃-d) δ 1.48 (s, 9H), 2.59 (s, 3H), 4.64 (s, 2H), 5.48 (s, 2H), 6.56 (d, J=7.91 Hz, 1H), 6.95 (d, J=8.20 Hz, 1H), 7.09 (t, J=8.05 Hz, 1H), 7.22 (dd, J=8.52, 1.73 Hz, 1H), 7.42 (s, 1H), 7.44-7.51 (m, 2H), 7.73 (dd, J=6.08, 3.44 Hz, 1H), 7.77-7.85 (m, 2H); MS (ESI+) 473.0 (M+H).

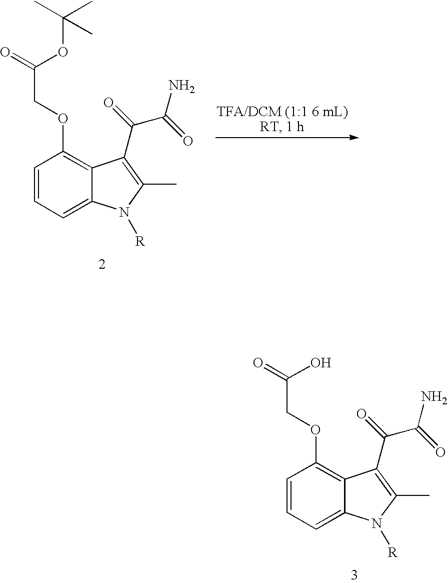

General procedure for the TFA-mediated cleavage of the tert-butyl ester of indole (2) to give 3: A suspension of the tert-butyl ester in DCM (3-5 mL) at RT was treated with TFA (2 mL). The mixture was stirred for 45 minutes before additional TFA (1 mL) was added. The reaction was further stirred for 15 minutes and quenched with water (3 mL). The solvent was concentrated and product extracted with DCM (3×25 mL). The combined extracts were washed with water, brine, decanted and concentrated. Purification by reverse phase semi-preparative HPLC eluting with a gradient mixture of Methanol (spiked with 0.1% formic acid) and Water (407 method ˜45:55) yielded the desired solid adduct.

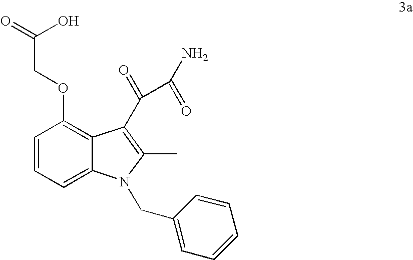

(3-Aminooxalyl-1-benzyl-2-methyl-1H-indol-4-yloxy)-acetic acid (3a): yield 65 mg, 75%; ¹H NMR (400 MHz, DMSO-d₆) δ 2.50 (s, 3H), 4.57 (s, 2H), 5.49 (s, 2H), 6.52 (d, J=7.71 Hz, 1H), 7.05 (t, J=7.83 Hz, 3H), 7.08-7.14 (m, 1H), 7.25 (t, J=7.25 Hz, 1H), 7.32 (t, J=7.30 Hz, 2H) 7.43 (s, 1H), 7.81 (s, 1H); MS (ESI+) 367.2 (M+H), 389.2 (M+Na⁺); HPLC (UV=100%), (ELSD=100%).

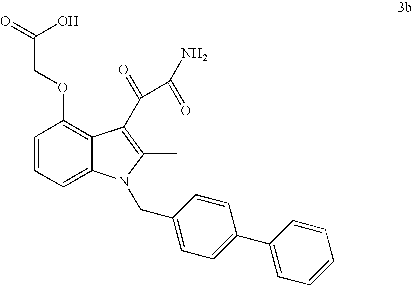

(3-Aminooxalyl-1-biphenyl-4-ylmethyl-2-methyl-1H-indol-4-yloxy)-acetic acid (3b): yield 90 mg, 92%; ¹H NMR (400 MHz, DMSO-d₆) δ 2.54 (s, 3H), 4.63 (s, 2H), 5.54 (s, 2H), 6.54 (d, J=7.66 Hz, 1H), 7.08 (t, J=7.88 Hz, 1H), 7.14 (d, J=6.98 Hz, 3H), 7.34 (t, J=7.10 Hz, 1H), 7.39-7.51 (m, 3H), 7.61 (d, J=7.76 Hz, 4H), 7.79 (s, 1H); MS (ESI+) 443.3 (M+H), 465.3 (M+Na⁺); HPLC (UV=100%), (ELSD=100%).

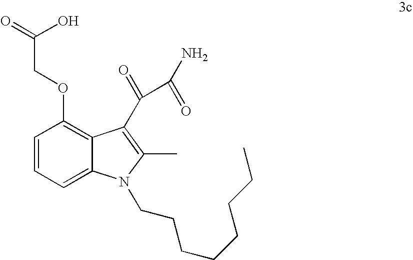

(3-Aminooxalyl-2-methyl-1-octyl-1H-indol-4-yloxy)-acetic acid (3c): yield 73 mg, 76%; ¹H NMR (400 MHz, DMSO-d₆) δ 0.80-0.90 (m, 3H), 1.17-1.39 (m, 12H), 1.60-1.75 (m, 2H), 2.55 (s, 3H), 4.16 (t, J=7.42 Hz, 2H), 4.61 (s, 2H), 6.52 (d, J=7.61 Hz, 1H), 7.08 (t, J=7.93 Hz, 1H), 7.12-7.17 (m, 1H), 7.38 (s, 1H), 7.73 (s, 1H); MS (ESI+) 389.3 (M+H), 411.3 (M+Na⁺); HPLC (UV=100%), (ELSD=100%).

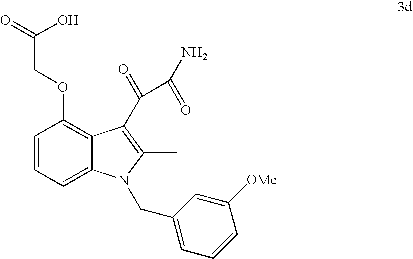

[3-Aminooxalyl-1-(3-methoxy-benzyl)-2-methyl-1H-indol-4-yloxy]-acetic acid (3d): yield 58.6 mg, 61%; ¹H NMR (400 MHz, DMSO-d₆) δ 2.50 (s, 3H), 3.70 (s, 3H), 4.60 (s, 2H), 5.46 (s, 2H), 6.53 (d, J=7.61 Hz, 2H), 6.66 (s, 1H), 6.83 (dd, J=8.22, 2.07 Hz, 1H), 7.06 (t, J=7.96 Hz, 1H), 7.09-7.15 (m, 1H), 7.22 (t, J=7.93 Hz, 1H), 7.41 (s, 1H), 7.80 (s, 1H); MS (ESI+) 397.1 (M+H); HPLC (UV=98.9%), (ELSD=98.8%).

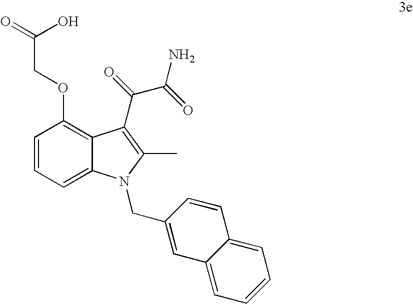

(3-Aminooxalyl-2-methyl-1-naphthalen-2-yl methyl-1H-indol-4-yloxy)-acetic acid (3e): yield 83 mg, 85.6%; ¹H NMR (400 MHz, DMSO-d₆) δ 2.56 (s, 3H), 4.64 (s, 2H), 5.66 (s, 2H), 6.55 (d, J=7.81 Hz, 1H), 7.06 (t, J=8.05 Hz, 1H), 7.18 (d, J=8.20 Hz, 1H), 7.24 (dd, J=8.52, 1.68 Hz, 1H), 7.44 (s, 1H), 7.46-7.52 (m, 2H), 7.58 (s, 1H), 7.78 (s, 1H), 7.83 (dd, J=6.10, 3.47 Hz, 1H), 7.85-7.91 (m, 2H); MS (ESI+) 417.2 (M+H); HPLC (UV=100%), (ELSD=100%).

### Example 17Z

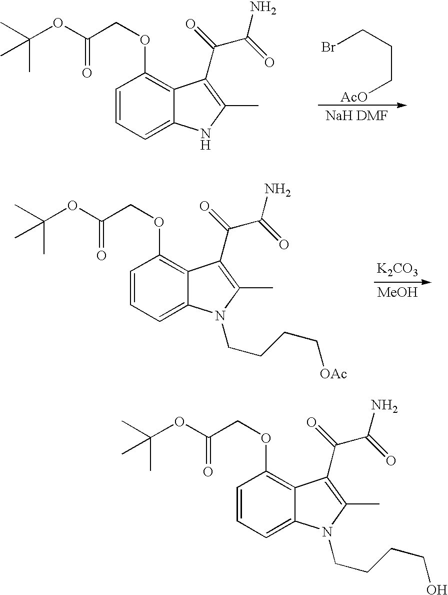

[1-(4-Acetoxy-butyl)-3-aminooxalyl-2-methyl-1H-indol-4-yloxy]-acetic acid tert-butyl ester. To a solution of indole (250 mg, 0.75 mmol) in DMF (3.8 mL) was added NaH (60% dispersion in mineral oil, 36 mg, 0.90 mmol). 4-Bromobutylacetate (0.12 mL, 0.83 mmol) was added after 25 min., and the reaction was quenched after 15 h. with saturated aq NH₄Cl (10 mL). The solution was extracted with EtOAc (0.1 L) and washed 3×20 mL H₂O, 20 mL saturated aq NaCl, dried over Na₂SO₄, filtered and concentrated in vacuo to give a brown oil. The oil was chromatographed over silica gel (12 g column, 0 to 80% EtOAc in heptane, over 40 min.) to give a mixture of starting indole (30 mg, 12%) and desired acetate (0.23 g, 70%) as a yellow solid. R_{f}=0.29 (25:75 heptane/EtOAc); ¹H NMR (400 MHz, CHLOROFORM-d) δ ppm 7.11-7.16 (m, J=8.1, 8.1 Hz, 1H) 6.96 (d, J=7.9 Hz, 1H) 6.54 (d, J=7.8 Hz, 1H) 4.60 (s, 2H) 4.08-4.14 (m, 4H) 2.59 (s, 3H) 2.05 (s, 3H) 1.81-1.90 (m, 2H) 1.72 (dd, J=8.8, 6.1 Hz, 2H) 1.47 (s, 9H).

[3-Aminooxalyl-1-(4-hydroxy-butyl)-2-methyl-1H-indol-4-yloxy]-acetic acid tert-butyl ester. To a solution of acetate (99 mg, 0.22 mmol) in DMF (0.70 mL) was added a saturated solution of K₂CO₃ in methanol (1.5 mL). The reaction was diluted with EtOAc (25 mL) after 1 h, washed with saturated aq NH₄Cl, dried over Na₂SO₄, filtered and concentrated in vacuo to give a yellow solid. The solid was chromatographed over silica gel (4 g column, 50 to 100% EtOAc in heptane, over 10 min., then EtOAc for 10 min.) to give the desired alcohol as a yellow solid (27 mg, 30%, 90% pure by HPLC). R_{f}=0.11 (EtOAc); ¹H NMR (400 MHz, CHLOROFORM-d) δ ppm 7.10-7.15 (m, 1H) 6.94-7.00 (m, J=8.2 Hz, 1H) 6.52-6.56 (m, 1H) 4.61 (s, 2H) 4.10-4.16 (m, 2H) 3.69 (t, J=6.2 Hz, 2H) 2.60 (s, 3H) 1.84-1.90 (m, J=3.1 Hz, 2H) 1.62-1.67 (m, 2H) 1.47 (s, 9H); MS (EI) 304 (M-CO₂t-Bu).

### Example 17AA

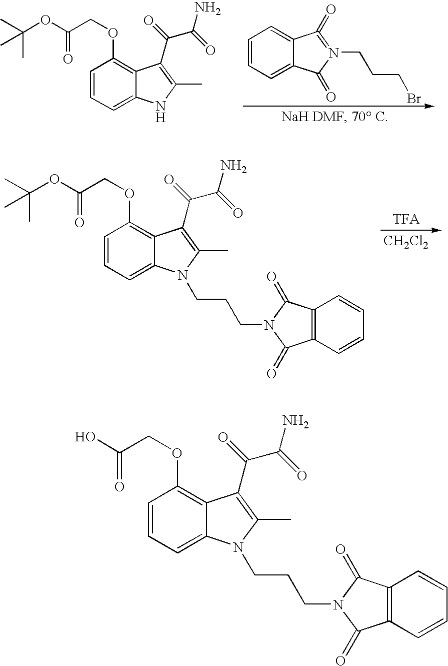

{3-Aminooxalyl-1-[3-(1,3-dioxo-1,3-dihydro-isoindol-2-yl)-propyl]-2-methyl-1H-indol-4-yloxy}-acetic acid tert-butyl ester. To a solution of indole (0.30 g, 0.90 mmol) in DMF (4.5 mL) was added NaH (60% dispersion in mineral oil, 43 mg, 1.1 mmol). After 30 min., alkyl bromide (0.27 g, 0.99 mmol) was added. Additional NaH (8.0 mg, 0.37 mmol) and alkyl bromide (0.050 g, 0.19 mmol) were added after 2 h and the reaction was heated to 70° C. The reaction was cooled to 25 70° C. after 16 h, diluted with EtOAc (0.1 L) and washed 3 □ 10 mL H₂O. The organic layer was washed with saturated (aq) NaCl, dried over Na₂SO₄, filtered and concentrated in vacuo to give a yellow solid. The solid was chromatographed over silica gel (12 g column, 25 to 100% EtOAc in heptane, over 35 min.) to give a mixture of starting indole (25 mg, 8%) and desired phthalimide (0.33 g, 70%) as a yellow solid. R_{f}=0.19 (25:75 heptane/EtOAc); ¹H NMR (400 MHz, METHANOL-d₄) δ ppm 7.82-7.86 (m, 2H) 7.78-7.82 (m, 2H) 7.06-7.13 (m, 2H) 6.51 (dd, J=7.0, 1.6 Hz, 1H) 4.62 (s, 2H) 4.25-4.32 (m, 2H) 3.80 (t, J=7.1 Hz, 2H) 2.60 (s, 3H) 2.12-2.21 (m, 4H) 1.45 (s, 9H).

{3-Aminooxalyl-1-[3-(1,3-dioxo-1,3-dihydro-isoindol-2-yl)-propyl]-2-methyl-1H-indol-4-yloxy}-acetic acid. To a solution of t-butyl ester (0.21 g, 0.40 mmol) in CH₂Cl₂ (6.1 mL) was added TFA (0.81 mL, 11 mmol). After 45 min., additional TFA (0.81 mL, 11 mmol) was added. The reaction was maintained at room temp. for an additional 45 min., then concentrated in vacuo to give a black oil. Water was added and the green suspension was concentrated in vacuo to give a green solid. The solid was dissolved in 1:9 DMSO/MeOH and chromatographed on a reverse phase HPLC (1% TEA buffer, 7 min., 28 mL/min, 5% MeOH in water to 100% MeOH over 5 min.) to give the product as a brown oil (0.11 g, 46%, 82% pure by reverse phase HPLC, UV detection). Note: The best purification method for this compound on reverse phase HPLC uses the 0728 method with 1% formic acid. However the poor solubility of the product in the presence of formic acid does not allow the use of the best purification method. ¹H NMR (400 MHz, METHANOL-d₄) δ ppm 7.85-7.88 (m, 2H) 7.79-7.82 (m, 2H) 7.00-7.09 (m, 2H) 6.57 (d, J=7.8 Hz, 1H) 4.49 (s, 2H) 4.26-4.31 (m, 2H) 3.80 (t, J=7.1 Hz, 2H) 2.61 (s, 3H) 2.17 (d, J=7.3 Hz, 2H); MS (EI) 464 (M+H).

### Example 17AB

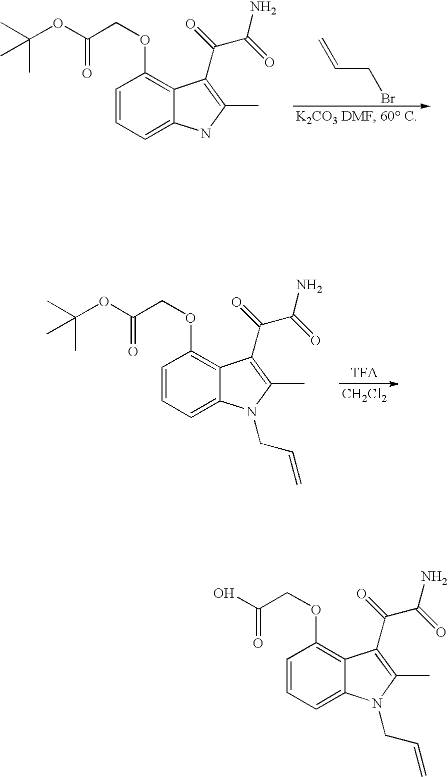

(1-Allyl-3-aminooxalyl-2-methyl-1H-indol-4-yloxy)-acetic acid tert-butyl ester. To a heated (60 □C) mixture of indole (0.11 g, 0.33 mmol) and K₂CO₃ (55 mg, 0.40 mmol) in DMF (1.7 mL) was added allyl bromide (0.030 mL). Additional allyl bromide (0.10 mL, 1.2 mmol) was added after 3 h and the reaction was maintained at 60° C. for 16 h before being cooled to 25° C. The mixture was then diluted into 15 mL EtOAc, washed 4×10 mL H₂O, 10 mL saturated aq NaCl, dried over Na₂SO₄, filtered and concentrated in vacuo to give a yellow solid. The solid was chromatographed over silica gel (4 g column, 50 to 75% EtOAc over 10 min., then 75% EtOAc for 15 min.) to give a mixture of starting indole (9 mg, 8%) and desired alkene (95 mg, 77%) as a yellow solid. R_{f}=0.24 (25:75 heptane/EtOAc); ¹H NMR (400 MHz, CHLOROFORM-d) δ ppm 7.12 (dd, J=8.1 Hz, 1H) 6.94 (d, J=8.2 Hz, 1H) 6.53 (d, J=7.9 Hz, 1H) 5.87-5.97 (m, 1H) 5.20 (dd, J=10.3, 0.7 Hz, 1H) 4.96 (dd, J=17.1, 0.6 Hz, 1H) 4.67-4.72 (m, 2H) 4.61 (s, 2H) 2.56 (s, 3H) 1.47 (s, 9H).

(1-Allyl-3-aminooxalyl-2-methyl-1H-indol-4-yloxy)-acetic acid. To a solution of t-butyl ester (75 mg, 0.40 mmol) in CH₂Cl₂ (6.0 mL) was added TFA (0.80 mL, 11 mmol). After 1 h, the reaction was concentrated in vacuo to give a brown oil. Water was added and the suspension was concentrated in vacuo to give a solid. The solid was dissolved in 1:9 DMSO/MeOH and chromatographed on a reverse phase HPLC (1% TEA buffer, 7 min., 28 mL/min, 5% MeOH in water to 100% MeOH over 5 min.) to give the acid as a pale white solid (7 mg, 11%). ¹H NMR (400 MHz, METHANOL-d₄) δ ppm 7.11 (t, J=8.1 Hz, 1H) 7.01 (d, J=7.8 Hz, 1H) 6.60 (d, J=7.7 Hz, 1H) 5.94-6.06 (m, 1H) 5.12-5.19 (m, 1H) 4.87-4.90 (m, 1H) 4.60 (s, 2H) 3.19 (q, J=7.4 Hz, 2H) 2.59 (s, 3H); MS (EI) 317 (M+H).

### Example 17AC

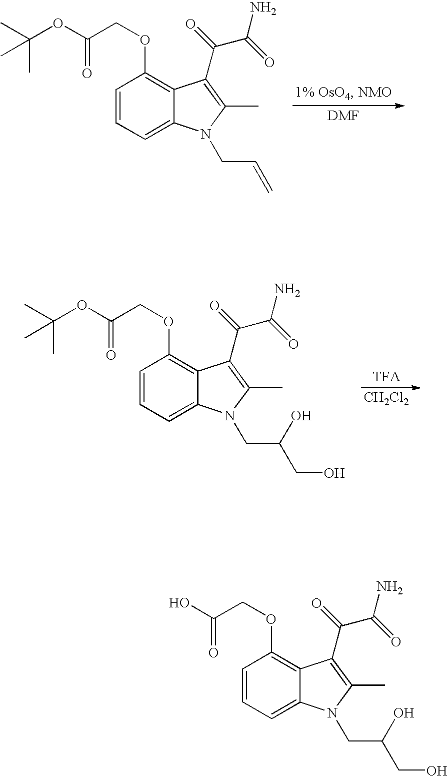

[3-Aminooxalyl-1-(2,3-dihydroxy-propyl)-2-methyl-1H-indol-4-yloxy]-acetic acid tert-butyl ester. To a solution of alkene (0.17 g, 0.45 mmol) and NMO (58 mg, 0.50 mmol) in DMF (2.5 mL) was added a solution of OsO₄ (0.030 mL, 4.5×10⁻⁶ mol, [0.16]) in H₂O. After 21 h, the reaction was loaded directly onto a silica gel column (12 g) and purified by flash chromatography (20 to 100% EtOAc in heptane over 20 min) to give the diol as a pale yellow solid (0.16 g, 85%). R_{f}=0.06 (1:1 heptane/EtOAc); ¹H NMR (400 MHz, METHANOL-d₄) δ ppm 7.10-7.18 (m, 2H) 6.54 (dd, J=7.3, 1.2 Hz, 1H) 4.65 (s, 2H) 4.38 (dd, J=14.8, 3.7 Hz, 1H) 4.14-4.22 (m, 1H) 3.96-4.03 (m, 1H) 3.61 (d, J=5.4 Hz, 1H) 2.99 (s, 2H) 2.86 (d, J=0.6 Hz, 1H) 2.66 (s, 3H) 1.46 (s, 9H).

[3-Aminooxalyl-1-(2,3-dihydroxy-propyl)-2-methyl-1H-indol-4-yloxy]-acetic acid. To a solution of t-butyl ester (0.16 g, 0.38 mmol) in CH₂Cl₂ (10 mL) was added TFA (1.6 mL, 0.020 mol). After 40 min., the reaction was concentrated in vacuo to give an oil. The oil was dissolved in 1:1H₂O/MeOH and chromatographed on a reverse phase HPLC (355 acid method) to give the acid as a pale yellow oil (4 mg, 3%). ¹H NMR (400 MHz, METHANOL-d₄) δ ppm 7.10-7.14 (m, 2H) 6.57-6.62 (m, 1H) 4.61 (s, 2H) 4.37 (dd, J=14.8, 3.8 Hz, 1H) 4.13-4.21 (m, 1H) 3.96-4.03 (m, 1H) 3.61 (d, J=5.4 Hz, 2H) 2.66 (s, 3H); MS (EI) 351 (M+H).

### Example 17 AD

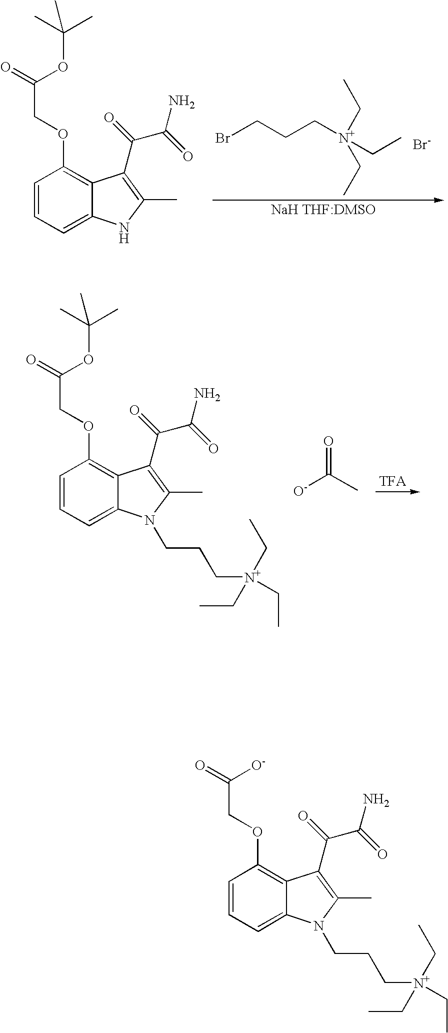

[3-(3-Aminooxalyl-4-tert-butoxycarbonylmethoxy-2-methyl-indol-1-yl)-propyl]-triethyl-ammonium acetate. To a solution of 3-aminooxalyl-2-methyl-1H-indo-4-yloxy)-acetic acid tert-butyl ester (50 mg, 0.15 mmol) in THF:DMSO [1:5] (0.5 mL: 2.5 mL) was added NaH (60% dispersion in mineral oil, 4 mg, 0.16 mmol) and the solution allowed to stir at room temperature. After 30 min, (3-Bromopropyl)triethylammonium bromide (80%, 30 mg, 0.08 mmol) was added and the mixture heated at 60° C. for 16 h. The reaction mixture was triturated 2×10 mL Et₂O. The oily residue was dissolved in CH₃OH:H₂O [1:1] (4 mL:4 mL) and purified by reverse phase HPLC starting with 5% CH₃OH in H₂O as the eluant. Concentration furnished 3-(3-aminooxalyl-4-tert-butoxycarbonylmethoxy-2-methyl-indol-1-yl)-propyl]-triethyl-ammonium acetate: yellow oil, 60.2 mg; LCMS 474 (M+); 1H NMR (400 MHz, METHANOL-d₄) δ ppm 7.05-7.25 (m, 2H), 6.57 (dd, J=6.96, 1.54 Hz, 1H), 4.66 (s, 2H), 4.26 (t, J=7.03 Hz, 2H), 3.17-3.29 (m, 8H), 2.60 (s, 3H), 2.07-2.19 (m, 2H), 1.94 (s, 3H), 1.47 (s, 9H), 1.18 (t, J=7.20 Hz, 9H).

[3-Aminooxalyl-1-(3-triethylammoniumpropyl)-2-methyl-1H-indol-4-yloxy]acetate. A solution of 3-(3-aminooxalyl-4-tert-butoxycarbonylmethoxy-2-methyl-indol-1-yl)-propyl]-triethyl-ammonium acetate (60.2 mg, 0.11 mmol) in triflouroacetic acid (2 mL) was prepared and allowed to stir at room temperature. After 30 min, H₂O (4 mL) was added and the mixture concentrated. The residue was redissovled in H₂O (4 mL) and concentrated again. The hazel residue was purified by reverse phase HPLC starting with 5% CH₃OH in H₂O as the eluant with 0.1% formic acid added to both eluant phases. Concentration furnished 3-Aminooxalyl-1-(3-triethylammoniumpropyl)-2-methyl-1H-indol-4-yloxy]acetate: yellow oil, 8.8 mg, (19%); LCMS 418 (M+); 1H NMR (400 MHz, METHANOL-d₄) δ ppm 8.33 (s, 1H), 7.13 (t, J=8.00 Hz, 1H), 7.04 (d, J=8.20 Hz, 1H), 6.61 (d, J=7.81 Hz, 1H), 4.58 (s, 2H), 4.19 (t, J=6.71 Hz, 2H), 3.12-3.29 (m, J=7.01, 7.01, 7.01 Hz, 8H), 2.58 (s, 3H), 2.11 (s, 2H), 1.17 (t, J=7.05 Hz, 9H).

### Example 17AE

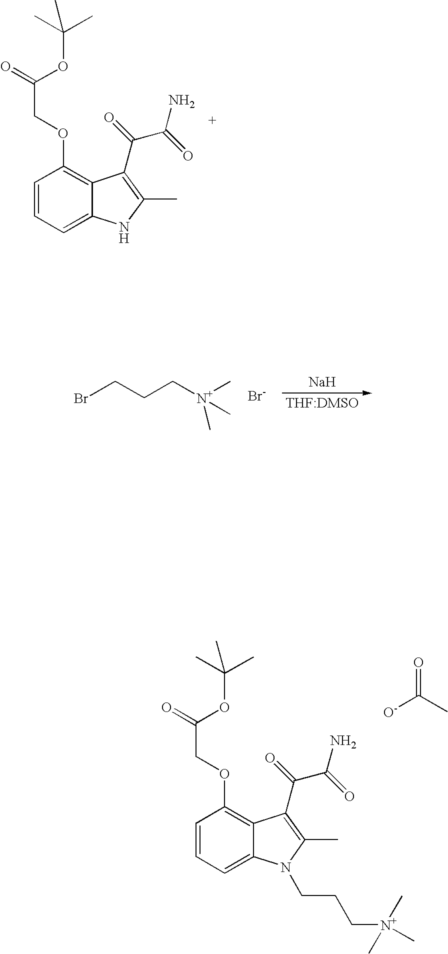

[3-(3-Aminooxalyl-4-tert-butoxycarbonylmethoxy-2-methyl-indol-1-yl)-propyl]-trimethyl-ammonium acetate. To a solution of 3-aminooxalyl-2-methyl-1H-indo-4-yloxy)-acetic acid tert-butyl ester (100 mg, 0.30 mmol) in THF:DMSO [1:5] (0.5 mL:2.5 mL) was added NaH (60% dispersion in mineral oil, 8 mg, 0.33 mmol) and the solution allowed to stir at room temperature. After 30 min, (3-bromopropyl)trimethylammonium bromide (60 mg, 0.33 mmol) was added and the mixture heated at 60° C. for 16 h. The reaction mixture was triturated 3×10 mL Et₂O. The oily residue was dissolved in CH₃OH and purified by reverse phase HPLC starting with 30% CH₃OH in H₂O as the initial eluant. Concentration furnished 3-(3-Aminooxalyl-4-tert-butoxycarbonylmethoxy-2-methyl-indol-1-yl)-propyl]-trimethyl-ammonium acetate: brown oil, 37 mg; LCMS 432 (M+); 1H NMR (400 MHz, METHANOL-d₄) δ ppm 7.07-7.22 (m, 2H), 6.57 (dd, J=7.08, 1.42 Hz, 1H), 4.66 (s, 2H), 4.26 (t, J=7.08 Hz, 2H), 3.35-3.48 (m, 2H), 3.09 (s, 9H), 2.61 (s, 3H), 2.19-2.32 (m, 2H), 1.94 (s, 3H), 1.47 (s, 9H).

### Example 17AF

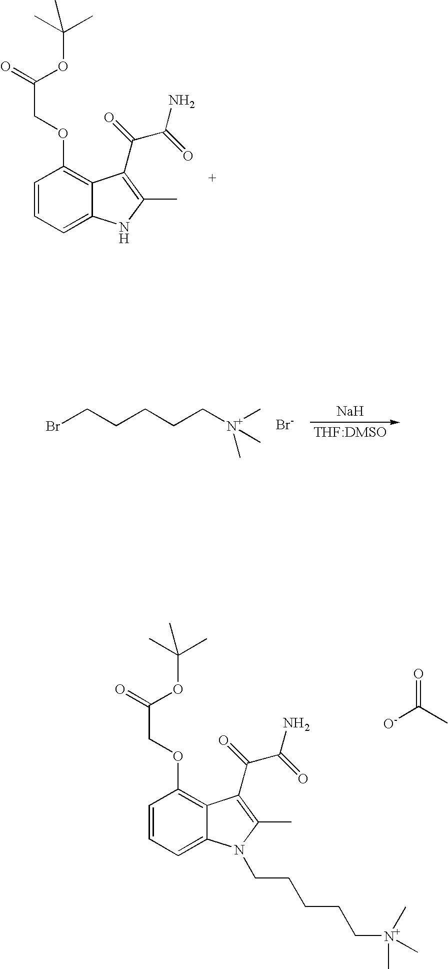

[5-(3-Aminooxalyl 4-tert-butoxycarbonylmethoxy-2-methyl-indol-1-yl)-pentyl]-trimethyl-ammonium acetate. To a solution of 3-aminooxalyl-2-methyl-1H-indo-4-yloxy)-acetic acid tert-butyl ester (200 mg, 0.60 mmol) in THF:DMSO [1:5] (1 mL:5 mL) was added NaH (60% dispersion in mineral oil, 16 mg, 0.66 mmol) and the solution allowed to stir at room temperature. After 45 min, (5-Bromopentyl)trimethylammonium bromide (191 mg, 0.66 mmol) was added and the mixture heated at 60° C. for 18 h. The reaction mixture was triturated 3×15 mL Et₂O. The brown gum was dissolved in CH₃OH:H₂O [1:1] (6 mL:6 mL) and purified by reverse phase HPLC starting with 10% CH₃OH in H₂O as the initial eluant. Concentration furnished 5-(3-Aminooxalyl-4-tert-butoxycarbonylmethoxy-2-methyl-indol-1-yl)-pentyl]-trimethyl-ammonium acetate: orange oil, 115.1 mg (37%); LCMS 460 (M+); 1H NMR (400 MHz, METHANOL-d₄) δ ppm 7.05-7.18 (m, 2H), 6.54 (dd, J=6.98, 1.56 Hz, 1H), 4.65 (s, 2H), 4.18 (t, J=7.13 Hz, 2H), 3.14-3.24 (m, 2H), 3.03 (s, 9H), 2.59 (s, 3H), 1.93 (s, 3H), 1.76-1.88 (m, 2H), 1.60-1.72 (m, 2H), 1.47 (s, 9H), 1.33-1.43 (m, 2H).

### Example 17AG

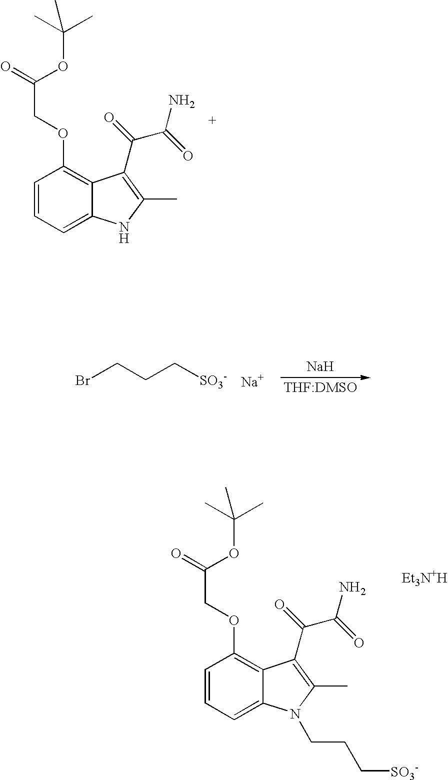

[Triethylammonium 3-(3-aminooxalyl-4-tert-butoxycarbonylmethoxy-2-methyl-indol-1-yl)-propane-1-sulfonate. To a solution of 3-aminooxalyl-2-methyl-1H-indo-4-yloxy)-acetic acid tert-butyl ester (200 mg, 0.60 mmol) in THF:DMSO [1:5] (1 mL: 5 mL) was added NaH (60% dispersion in mineral oil, 16 mg, 0.66 mmol) and the solution allowed to stir at room temperature. After 45 min, bromopropanesulfate, sodium salt (149 mg, 0.66 mmol) was added and the mixture heated at 60° C. for 18 h. The reaction mixture was triturated 4×15 mL Et₂O. The brown gum was dissolved in CH₃OH (14 mL) and purified by reverse phase HPLC starting with 5% CH₃OH in H₂O as the initial eluant with 0.1% triethylamine added to both eluant phases. Concentration provided Triethylammonium 3-(3-aminooxalyl-4-tert-butoxycarbonylmethoxy-2-methyl-indol-1-yl)-propane-1-sulfonate: 41.5 mg (12%); LCMS 399 (M-tBu+H); 1H NMR (400 MHz, METHANOL-d₄) δ ppm 7.20-7.27 (m, 1H), 7.14 (t, J=8.00 Hz, 1H), 6.55 (d, J=7.81 Hz, 1H), 4.65 (s, 2H), 4.35-4.44 (m, 2H), 3.13 (q, J=7.22 Hz, 4H), 2.89 (t, J=7.22 Hz, 2H), 2.65 (s, 3H), 2.17-2.27 (m, 2H), 1.46 (s, 9H), 1.25 (t, J=7.32 Hz, 6H).

### Example 17 AH

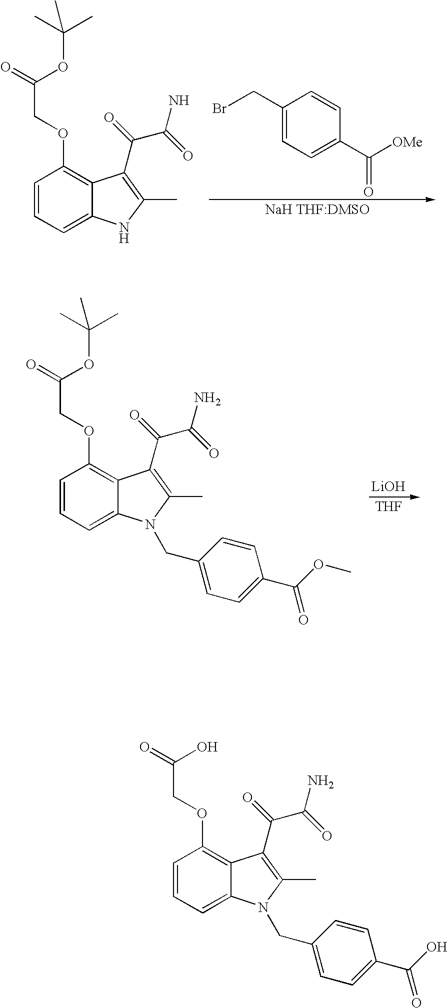

4-(3-Aminooxalyl-4-tert-butoxycarbonylmethoxy-2-methyl-indol-1-ylmethyl)-benzoic acid methyl ester. To a solution of 3-aminooxalyl-2-methyl-1H-indo-4-yloxy)-acetic acid tert-butyl ester (50 mg, 0.15 mmol) in THF:DMSO [1:5] (0.5 mL: 2.5 mL) was added NaH (60% dispersion in mineral oil, 4 mg, 0.16 mmol) and the solution allowed to stir at room temperature. After 30 min, 4-bromomethylbenzoic acid, methyl ester (38 mg, 0.17 mmol) was added and the mixture stirred at rt for 18 h. The reaction mixture was partitioned between ethyl acetate (30 mL) and H₂O (30 mL) and the aqueous phases extracted 2×30 mL ethyl acetate. The combined organic phases were washed successively with H2O (20 mL), then saturated aqueous NaCl solution (20 mL) and dried over MgSO₄. Concentration gave a pale yellow oil that was further purified by reverse phase HPLC starting with 30% CH₃OH in H₂O as the initial eluant to afford 4-(3-aminooxalyl-4-tert-butoxycarbonylmethoxy-2-methyl-indol-1-ylmethyl)-benzoic acid methyl ester which was used without further purification: 39.2 mg (60%); 1H NMR (400 MHz, METHANOL-d₄) δ ppm 7.90-7.98 (m, 2H), 7.17 (d, J=8.25 Hz, 2H), 7.11 (t, J=8.05 Hz, 1H), 7.01 (d, J=8.25 Hz, 1H), 6.57 (d, J=7.81 Hz, 1H), 5.56 (s, 2H), 4.68 (s, 2H), 3.87 (s, 3H), 2.56 (s, 3H), 1.46 (s, 9H).

4-(3-Aminooxalyl-4-carboxymethoxy-2-methyl-indol-1-ylmethyl)-benzoic acid. To an ice-cold suspension of 4-(3-aminooxalyl-4-tert-butoxycarbonylmethoxy-2-methyl-indol-1-ylmethyl)-benzoic acid methyl ester (39.2 mg, 0.08 mmol) in THF (5 mL) was added a 0.2 M aqueous LiOH solution (1.2 mL, 0.24 mmol) and the solution allowed to stir at) 0° C. for 30 min, then at room temperature for 18 h. The mixture was then treated with an ice-cold 0.2 N HCl in a saturated aqueous NaCl solution (15 mL) and the mixture extracted with CH₂Cl₂ (3×10 mL). The combined organic phases were washed with saturated aqueous NaCl solution (10 mL) and concentrated to a yellow solid. Purification by reverse phase HPLC furnished 4-(3-Aminooxalyl-4-carboxymethoxy-2-methyl-indol-1-ylmethyl)-benzoic acid: 3 mg; ¹H NMR (400 MHz, METHANOL-d₄) δ 2.57 (s, 3H), 4.60 (s, 2H), 5.54 (s, 2H), 6.61 (d, J=8.00 Hz, 1H), 6.97 (d, J=8.10 Hz, 1H), 7.09 (t, J=8.03 Hz, 1H), 7.15 (d, J=8.35 Hz, 2H), 7.94 (d, J=8.35 Hz, 2H); MS (ESI+) 411.2 (M+H); HPLC (UV=98.4%), (ELSD=96.6%).

### Example 17AI

2-(3-Aminooxalyl-1-benzyl-2-methyl-1H-indol-4-yloxy)-propionic acid (XXII); J. Med. Chem. 1996, 39, 5159-5157 In a two dram vial combine 2-(1-Benzyl-4-hydroxy-2-methyl-1H-indol-3-yl)-2-oxo-acetamide (XX) (0.2000 g, 0.000649 mol, 1 eq), potassium carbonate (0.0986 g, 0.000714 mol, 1.1 eq), methyl bromopropionate (0.080 mL, 0.000714 mol, 1.1 eq) and DMF (4 mL). Stir reaction mixture and heat to 75° C. overnight.

After this time the reaction was cooled to room temperature. The crude material was diluted with methanol (6 mL) and purified by HPLC. LCMS m/e 395 (M+H). Amount of 2-(3-aminooxalyl-1-benzyl-2-methyl-1H-indol-4-yloxy)-propionic acid methyl ester (XXI) isolated: 55.2 mg (0.00014 mol, yield=22%). ¹H NMR (400 MHz, CHLOROFORM-d) δ ppm 7.28-7.35 (m, 3H), 7.01-7.11 (m, 3H), 6.92 (d, J=8.10 Hz, 1H), 6.49 (d, J=7.91 Hz, 1H), 5.33 (s, 2H), 4.93 (q, J=6.82 Hz, 1H), 3.72 (s, 3H), 2.54 (s, 3H), 1.70 (d, J=6.83 Hz, 3H).

In a 100 mL round bottom flask combine 2-(3-Aminooxalyl-1-benzyl-2-methyl-1H-indol-4-yloxy)-propionic acid methyl ester (XXI) (0.0723 g, 0.000183 mol, 1 eq), ethanol (21 mL), THF (7 mL) and 2N sodium hydroxide (1.741 mL, 0.003483 mol, 19 eq). The reaction was stirred at room temperature for two and half hours. After this time the reaction was acidified with 1N HCl and extracted with ethyl acetate (three times 15 mL). Thorough drying provided product. LCMS m/e 403 (M+Na). Amount of XXII isolated: 43.6 mg (0.000115 mol, yield=63%). ¹H NMR (400 MHz, METHANOL-d₄) δ ppm 7.19-7.37 (m, 4H), 7.00-7.13 (m, 3H), 6.57 (d, J=7.76 Hz, 1H), 5.48 (s, 2H), 4.95 (q, J=6.65 Hz, 1H), 2.55 (s, 3H), 1.70 (d, J=6.88 Hz, 3H).

### Example 17 AJ

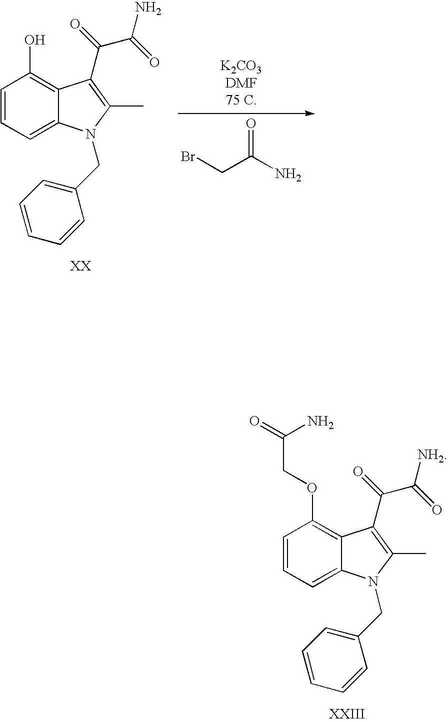

2-(1-Benzyl-4-carbamoylmethoxy-2-methyl-1H-indol-3-yl)-2-oxo-acetamide (XXIII) In a two dram vial combine 2-(1-Benzyl-4-hydroxy-2-methyl-1H-indol-3-yl)-2-oxo-acetamide (XX) (0.3000 g, 0.000973 mol, 1 eq), potassium carbonate (0.1479 g, 0.00107 mol, 1.1 eq), bromoacetamide (0.1477 g, 0.00107 mol, 1.1 eq) and DMF (4 mL). Stir reaction mixture and heat to 75° C. overnight. After this time the reaction was cooled to room temperature. The crude material was diluted with methanol (6 mL) and purified by HPLC. LCMS m/e 366 (M+H). HPLC purity 97%. Amount of XXIII isolated: 69.8 mg (0.000191 mol, yield=20%). ¹H NMR (400 MHz, METHANOL-d₄) δ ppm 7.22-7.36 (m, 3H), 7.12 (t, J=8.05 Hz, 1H), 7.06 (d, J=6.74 Hz, 2H), 6.88 (d, J=8.15 Hz, 1H), 6.62 (dd, J=7.93, 0.71 Hz, 1H), 5.48 (s, 2H), 2.64 (s, 3H), 2.15 (s, 2H).

Certain such azaindole and azaindole related compounds were evaluated for phospholipase activity using the protocol of Example 12. The results are shown in Table 8.

TABLE 8
Inhibition of pancreas secreted human, mouse and porcine PLA₂

| | | | ILYPSA | | | ILYPSA % | | |
|---|---|---|---|---|---|---|---|---|
| | | | IC50 (μM) | | | inhibition at 15 μM | | |
| | | | | pps | mps | hps | pps | mps |
| structure | Compound | MW | hps PLA₂ | PLA₂ | PLA₂ | PLA₂ | PLA₂ | PLA₂ |
| 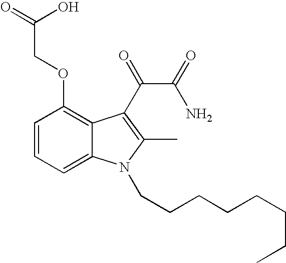 | ILY-V-2 | 388.46 | 0.64 | 0.03 | 0.21 | | | |
| 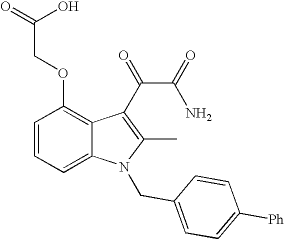 | ILY-V-3 | 442.46 | 1.26 | 0.21 | 0.77 | | | |
| 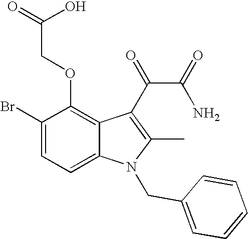 | ILY-IV-3 | 445.26 | | 5.87 | | 35.77 | | 39.75 |
| 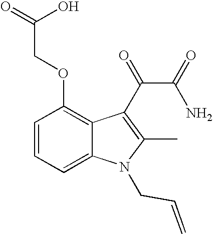 | ILY-V-10 | 316.31 | | 0.46 | 3.21 | 64 | | |
| 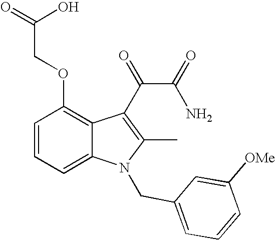 | ILY-V-8 | 396.39 | 3.25 | 0.04 | 0.97 | | | |
| 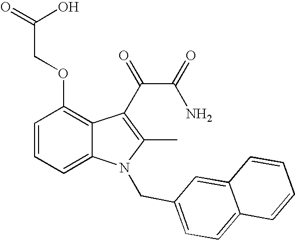 | ILY-V-9 | 416.43 | 2.11 | 0.06 | 0.71 | | | |
| 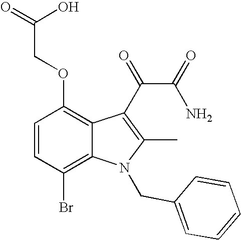 | ILY-IV-9 | 445.26 | 0.54 | 0.04 | 0.36 | | | |
| 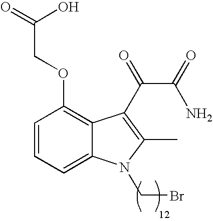 | ILY-V-22 | 523.46 | 1.22 | 0.52 | 1.01 | | | |
| 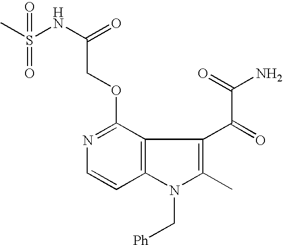 | ILY-IV-21 | 443.47 | 1.16 | <0.02 | 0.32 | | | |
| 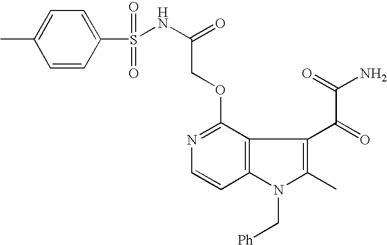 | ILY-IV-26 | 519.57 | >5.00 | 0.08 | 3.09 | | | |
| 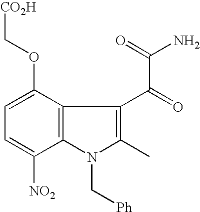 | ILY-IV-29 | 411.36 | 1.29 | 0.02 | 0.33 | | | |
| 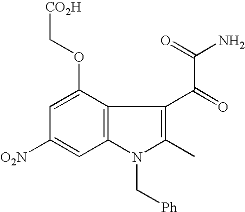 | ILY-IV-34 | 411.36 | 2.8 | 0.07 | 1.08 | | | |
| 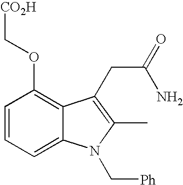 | ILY-III-22 | 352.38 | 1.32 | 0.16 | 0.89 | | | |
| 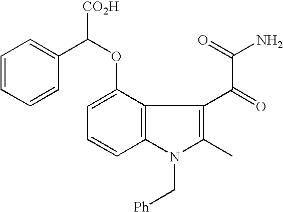 | ILY-IV-32 | 442.48 | | | | 41.73 | 38.5 | 47.49 |
| 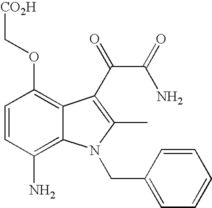 | ILY-IV-35 | 381.38 | 8.84 | 0.32 | 1.74 | | | |
| 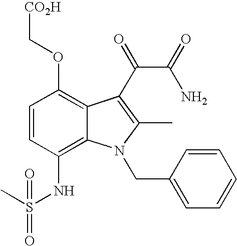 | ILY-IV-37 | 459.47 | 3.48 | 0.28 | 1.02 | | | |

### Example 18

### MOUSE PHARMACOKINETIC STUDY

The plasma exposure of male CD-1 mice to indole and indole-related test articles (TAs) following intravenous (IV, 3 mg/kg) and oral (PO, 30 mg/kg) routes of administration was measured. This model was used to investigate the bioavailability of indole and indole-related TAs in mouse. Mice were selected for the study since they are an accepted species frequently used in pre-clinical evaluation of drugs intended for human use.

Male CD-1 mice (7-8 weeks old) were obtained from Charles River Laboratories (Wilmington, Mass.). Two groups (N=18 and 27) of male CD-1 mice were used for the study. Upon arrival, the animals were placed on Rodent Diet 5001 (Purina Mills, Inc., St. Louis, Mo.).

On study day (−1), indole and indole-related TAs were formulated for oral or IV dosing by mixing the formulation components with test article in the proportions described in Table 10.1. The components were mixed by vortexing and sonicating in a warming bath for 60 minutes. Animals were fasted overnight prior to start of the study. On study day (1), formulations were sonicated for an hour to make sure that no visible particles were present prior to dosing, or if present were evenly distributed in suspension. Formulated test article were stirred continuously during dosing.

TABLE 10.1
Oral and IV Dose Formulations

| | |
|---|---|
| PO | IV |

| | | | | |
|---|---|---|---|---|
| H₂O | 85 | ml | 60 | ml |
| PEG400 | 9 | ml | | |
| PEG300 | | | 30 | ml |
| Tween-80 | 50 | ul | | |
| Ethanol | | | 5 | ml |
| DMSO | 5 | ml | 5 | ml |
| CMC | 900 | mg | | |
| Test Article | 300 | mg | 60 | mg |

All animals were weighed on study day (1) and the body weights were recorded and used for dose calculation. The animals were dosed by either PO or IV route as outlined in Table 10.2. Blood samples (0.5 mL) were collected at specified time intervals into labeled, yellow-capped Microtainer tubes. The tubes were centrifuged (8,000×g, 10 min). Serum was then pipetted off into labeled Eppendorf® tubes and frozen at −80° C. Clinical observations were recorded as needed.

TABLE 10.2
Oral and IV Dosing Schedule

| | | | | Mice Per |
|---|---|---|---|---|
| Compound | Group No. | Dose | Time Points | Time Point |
| Test Article | 1 | PO | 0.5 h, 1 h, 1.5 h, | 3 |
| | | (30 mg/kg) | 2 h, 6 h, 24 h | |
| Test Article | 2 | IV | 5 m, 10 m, 20 m, | 3 |
| | | (3 mg/kg) | 30 m, 45 m, 1 h, | |
| | | | 2 h, 6 h, 24 h | |

Analysis of serum samples was performed by LC/MS/MS (Waters Quattro Premier, Milford, Mass.). The Limit Of Quantitation (LOQ) for each compound is listed in Table 10.3. Areas under curves (AUC) were calculated using Graphpad Prism Version 4. Bioavailability was calculated using the following equation:
(Bioavailability)=(AUC_{0-t,oral}/AUC_{0-t,iv})×(Doseᵢᵥ/Doseₒᵣₐₗ)×100

where AUC₀₋ₜ=total area under curve at the last measurable time point

Based on the serum levels analyzed by LC/MS/MS, the calculated bioavailability of indole and indole-related TAs in CD-1 mice is summarized in Table 10.3.

TABLE 10.3
Bioavialability of Compounds

| Compound | Bioavailability (%) | LOQ (ng/ml) |
|---|---|---|
| ILY-V-26 | 0.00 | 200 |
| ILY-V-30 | 0.00 | 120 |
| ILY-V-32 | 0.00 | 200 |
| ILY-IV-40 | 0.50 | 3 |
| ILY-V-37 | 0.15 | 45 |
| ILV-V-27 | 1.49 | 60 |
| ILY-V-41 | 1.62 | 45 |
| ILV-V-31 | 5.15 | 45 |
| ILY-V-33 | 8.75 | 120 |
| ILY-II-1 | 11.00 | 1 |
| ILY-II-14 | 14.74 | 16 |

### Example 19

### MOUSE DIET-INDUCED OBESITY

The high-fat diet-fed C57BL/6J mouse model of human diabetes, originally introduced by Surwit and colleagues (Surwit, R S, et al. (1988) “Diet-induced type II diabetes in C57BL/6J mice”, Diabetes 37: 1163-1167) is a widely accepted, clinically relevant, polygenic model that induces obesity, dyslipidemia, glucose- and insulin-resistance as early as 3 weeks after commencing the high fat diet (Winzell, M S and Ahren, B (2004) “The high-fat diet-fed mouse: a model for studying mechanisms and treatment of impaired glucose tolerance and type 2 diabetes”, Diabetes 53 Suppl 3: S215-219). This model was used to investigate the effects of indole and indole-related Test Articles. Avandia (rosiglitazone) was used as a control Test Article.

Female C57Black/6J mice (5-6 weeks old) were obtained from Jackson laboratories (Bar Harbor, Me.). Upon arrival, the animals were placed on Laboratory Rodent Diet 5001 (Purina Mills, Inc., St. Louis, Mo.). Diet and water was provided ad libitum throughout the course of the study. Animals were acclimated for at least seven days, and then randomized by weight into twelve groups of eight animals each. Each group of animals was placed on diets with and without Test Articles as described in Table 11. All diets other than Laboratory Rodent Diet 5001 were provided by Research Diets (New Brunswick, N.J.).

In these studies and the accompanying figures, Diet D12328 from Research Diets is referred to as the “Low Fat” or Control diet/chow, while Diet D12331 from Research Diets is referred to as the “High Fat” diet. Groups 1-6 were fed diet D12328 that contained either no drug (Group 1) or varying amounts of Test Articles (Groups 2-6). Groups 7-12 were fed diet D12331 that contained either no drug (Group 7) or varying amounts of Test Articles (Groups 8-12). The Test Article content was calculated such that ad libitum consumption by the animals would deliver doses (in mg of Test Article per kg animal weight per day) approximating those listed in Table 11.

In this and other examples, Test Article ILY4008 is compound ILY-V-26 (5-26), Test Article ILY4013 is compound ILY-V-32 (5-32), Test Article ILY4011 is compound ILY-V-30 (5-30), and Test Article ILY4016 is compound ILY-IV-40 (4-40).

TABLE 11
Mouse Diet-Induced Obesity Assay Diets

| Group | Diet | Added Test Article |
|---|---|---|
| 1 | D12328 | No added Test Article |
| 2 | D12328 | 50 mg/kg/d Rosiglitazone |
| 3 | D12328 | 90 mg/kg/d ILY4008 or ILY4013 |
| 4 | D12328 | 25 mg/kg/d ILY4008 or ILY4013 |
| 5 | D12328 | 90 mg/kg/d ILY4011 or ILY4016 |
| 6 | D12328 | 25 mg/kg/d ILY4011 or ILY4016 |
| 7 | D12331 | No added Test Article |
| 8 | D12331 | 50 mg/kg/d Rosiglitazone |
| 9 | D12331 | 90 mg/kg/d ILY4008 or ILY4013 |
| 10 | D12331 | 25 mg/kg/d ILY4008 or ILY4013 |
| 11 | D12331 | 90 mg/kg/d ILY4011 or ILY4016 |
| 12 | D12331 | 25 mg/kg/d ILY4011 or ILY4016 |

Animals were maintained on the diets for up to eleven weeks. Body weights were recorded weekly. Blood was drawn within 1-2 hrs of lights-on, without fasting. The serum was analyzed for glucose, total cholesterol, triglycerides (TG) and lysophospholipid (LPC) content.

Statistical analyses were performed using GraphPad Prism 4.03. (GraphPad Software, Inc., San Diego, Calif.). Two sets of statistical analyses were performed. First, the Low Fat Chow, no treatment group was compared by student's two-tailed T-test against the High Fat, High Sucrose diet, no treatment group. In all figures an “a” above the low fat chow, no treatment column signifies that the values are significantly different (p<0.05) from the High Fat, High Sucrose diet, no treatment group. Second, all treatment groups on the High Fat, High Sucrose diet were compared to the no-treatment group on that diet by 1-way ANOVA, followed by a Dunnett's post-test. A “b” above a graph column signifies that the values are significantly different (p<0.05) versus the no-treatment group on that diet.

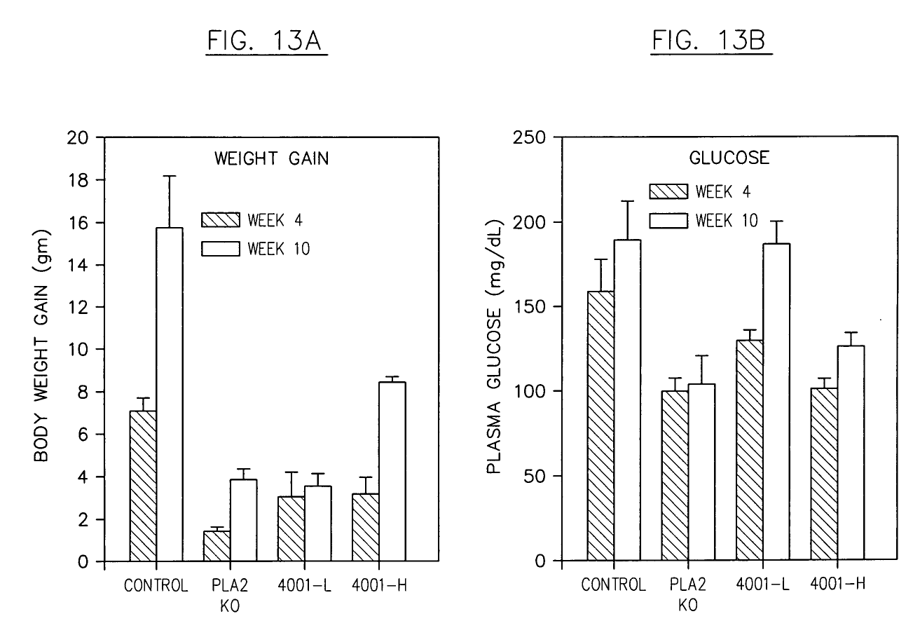

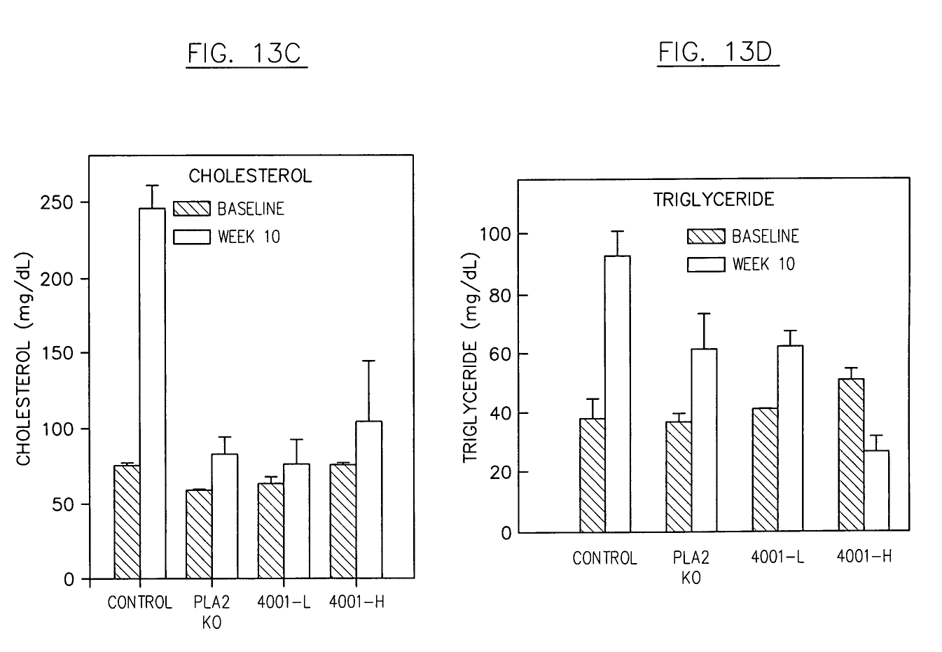

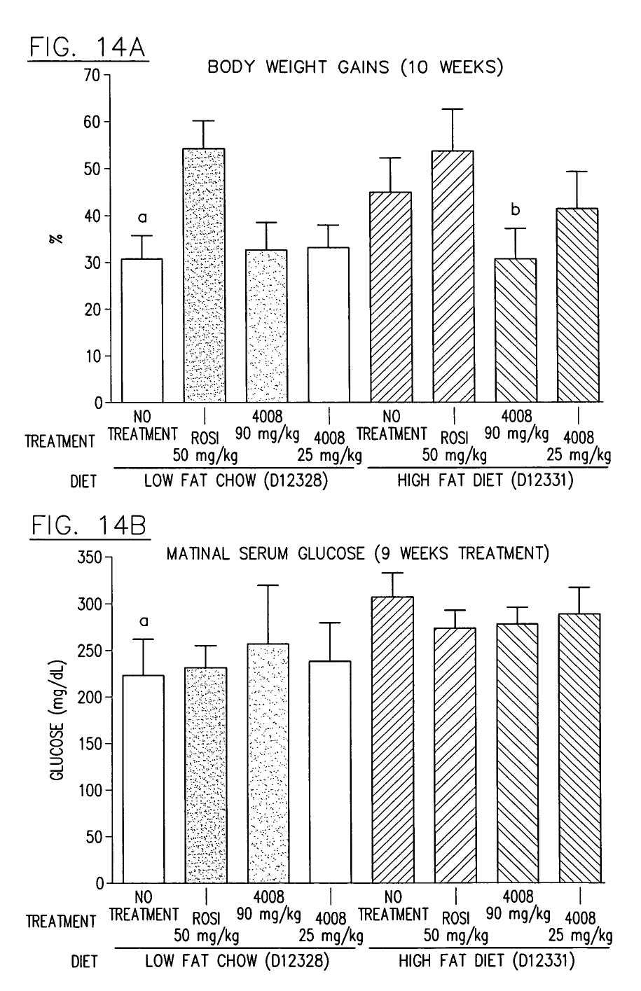

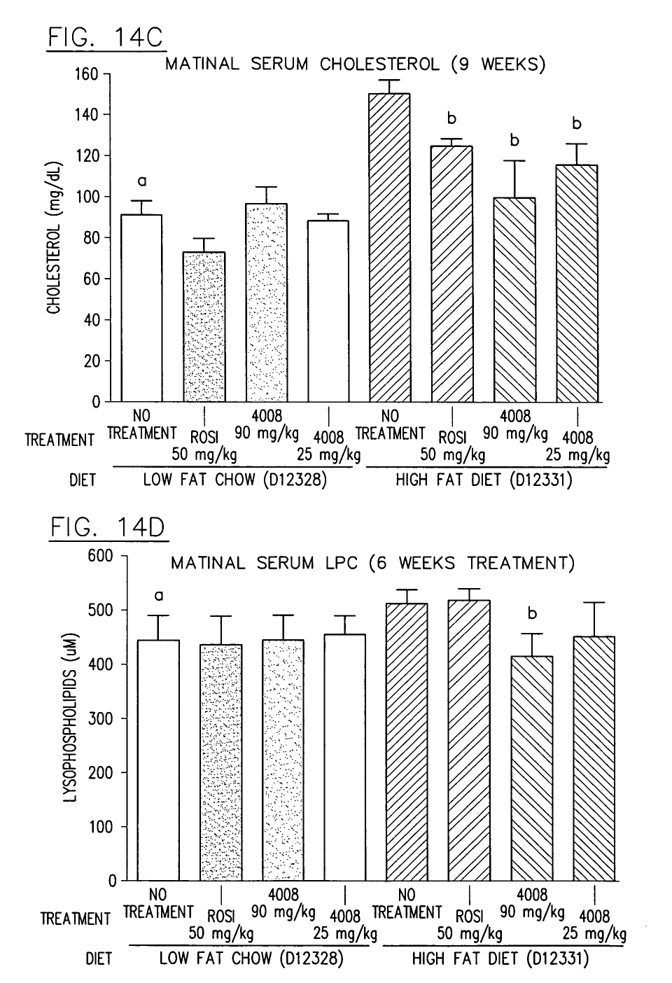

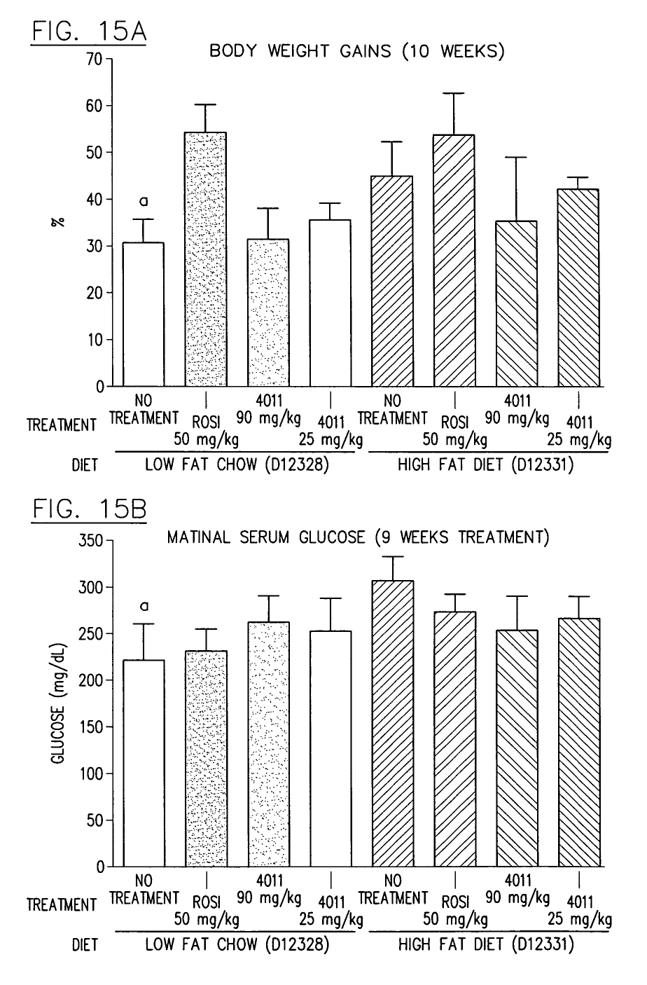

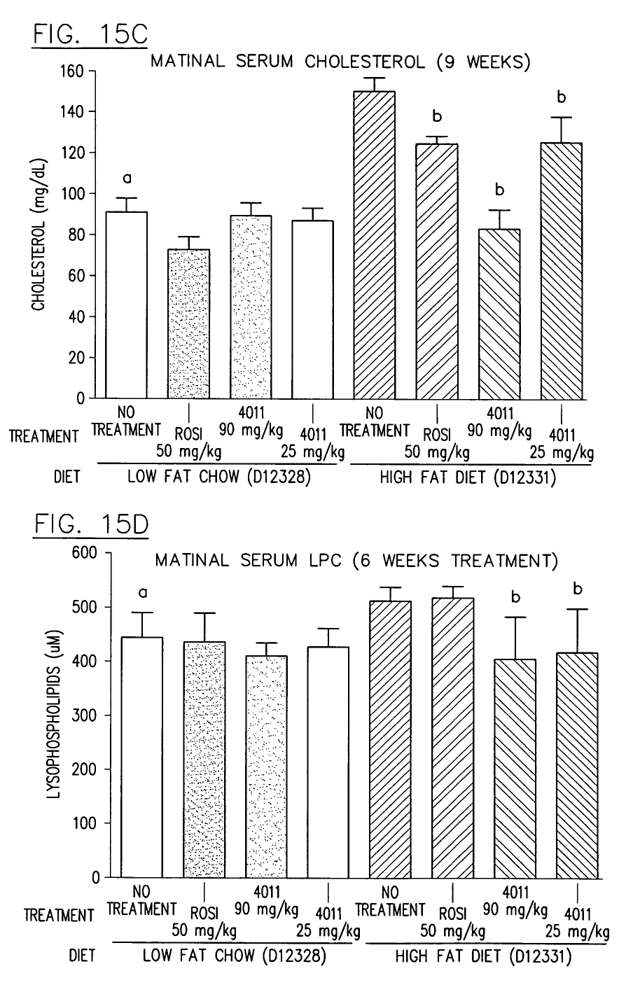

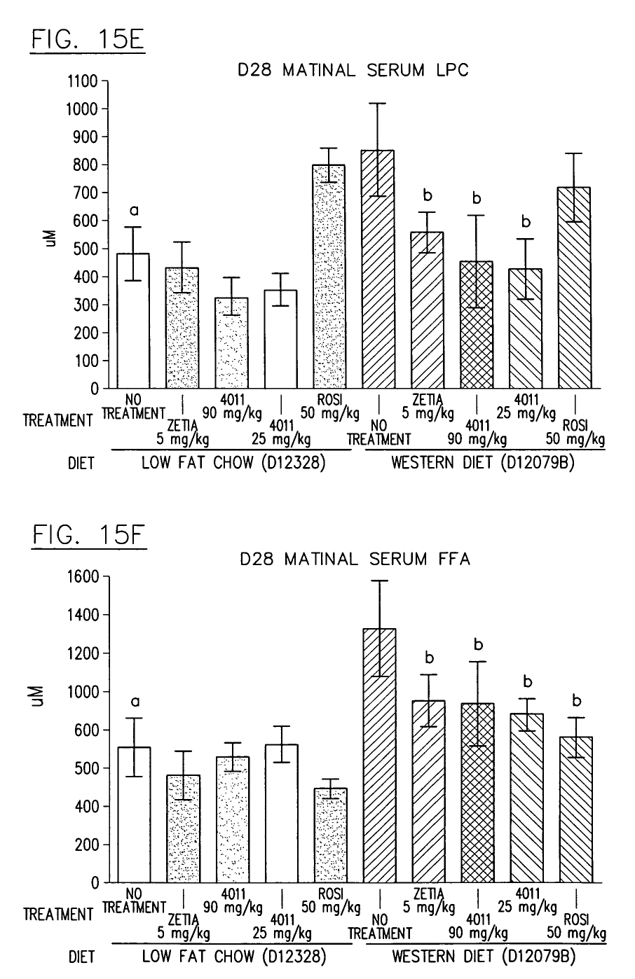

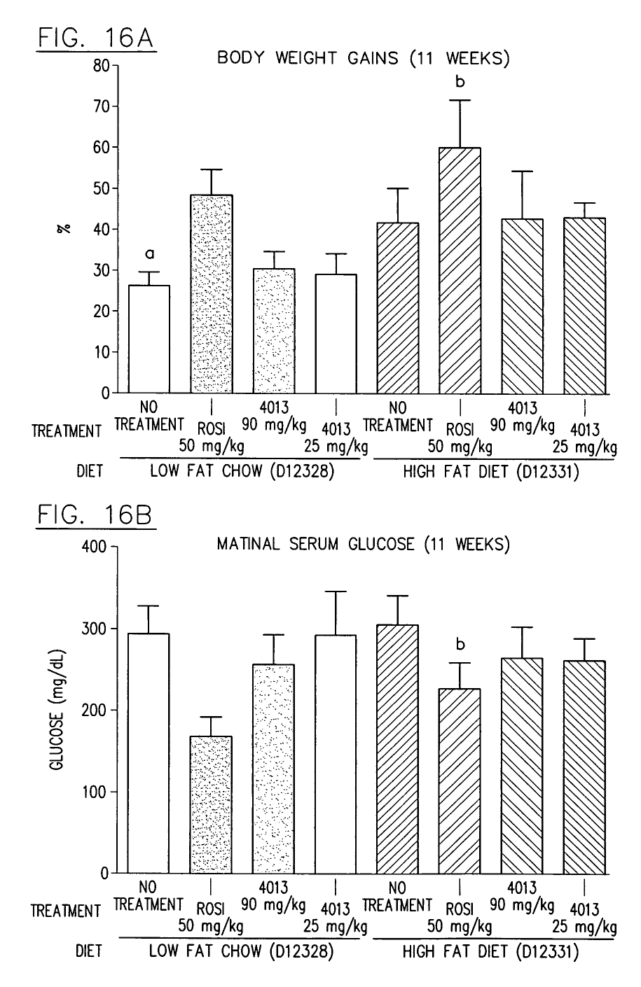

Results for Test Article ILY4008 (ILY-V-26) are shown in FIGS. 14A, 14B, 14C and 14D. Results for Test Article ILY4011 (ILY-V-30) are shown in FIGS. 15A, 15B, 15C and 15D. Results for Test Article ILY4013 (ILY-V-32) are shown in FIGS. 16A, 16B and 16C. Results for Test Article ILY4016 (ILY-IV-40) are shown in FIGS. 17A, 17B, and 17C.

No or little effect was observed when animals fed a low fat control diet were compared to animals fed a low fat control diet containing ILY4008, ILY4011, ILY4013 or ILY4016. This observation suggests that some embodiments provide efficacy under high-risk diet conditions yet have no observable effect under lower risk diet conditions.

### Example 20

### LDL RECEPTOR KNOCKOUT MICE

Mice lack an enzyme found in humans, cholesterol ester transfer protein (CETP), which is responsible for the transfer of cholesterol from high density lipoproteins (HDL) to the ApoB-containing lipoproteins such as very low density lipoproteins (VLDL) and low density lipoproteins (LDL). Consequently, LDL cholesterol levels in wild-type mice are very low compared to those seen in humans. The low density lipoprotein receptor (LDLR) is involved with clearing LDL and lipoprotein remnants containing apoE. If the LDLR is inactivated, LDL cholesterol levels rise to levels seen in humans. On a normal rodent diet, the LDL cholesterol levels in LDLR deficient mice are elevated compared to wild-type mice. If the LDLR deficient mice are fed a Western-type diet containing elevated levels of fats and cholesterol, then the total cholesterol and LDL cholesterol levels become highly elevated and can exceed 1000 mg/dL and 300 mg/dL, respectively. This model was used to investigate the effects of indole and indole-related Test Articles. Avandia (rosiglitazone) and Zetia (ezetimibe) were used as control test articles.

Male LDL receptor knockout mice (B6.129S7-Ldlrtm1Her) were obtained from Jackson Labs (Bar Harbor, Me.). Upon arrival, the animals were placed on Laboratory Rodent Diet 5001 (Purina Mills, Inc., St. Louis, Mo.). Diet and water was provided ad libitum throughout the course of the study. Animals were acclimated for at least seven days, and then randomized by body weight into fourteen groups of seven animals each. Each group of animals was placed on diets with and without Test Articles as described in Table 12. All diets other than Laboratory Rodent Diet 5001 were provided by Research Diets (New Brunswick, N.J.).

In these studies and the accompanying figures, Diet D12328 from Research Diets is referred to as the “Low Fat” or Control diet, while Diet D12079B from Research Diets is referred to as the “Western” diet. Groups 1-7 were fed diet D12328 that contained either no drug (Group 1) or varying amounts of Test Articles (Groups 2-7). Groups 8-14 were fed diet D12079 that contained either no drug (Group 8) or varying amounts of Test Articles (Groups 9-14). The Test Article content was calculated such that ad libitum consumption by the animals would deliver doses (in mg of Test Article per Kg animal weight per day) approximating those listed in Table 12.

TABLE 12
LDL Receptor Knockout Mice Assay Diets

| Group | Diet | Added Test Article |
|---|---|---|
| 1 | D12328 | No added Test Article |
| 2 | D12328 | 5 mg/kg/day ezetimibe |
| 3 | D12328 | 90 mg/kg/d ILY4008 or ILY4013 |
| 4 | D12328 | 25 mg/kg/d ILY4008 or ILY4013 |
| 5 | D12328 | 90 mg/kg/d ILY4011 or ILY4016 |
| 6 | D12328 | 25 mg/kg/d ILY4011 or ILY4016 |
| 7 | D12328 | 50 mg/kg/d Rosiglitazone |
| 8 | D12079B | No added Test Article |
| 9 | D12079B | 5 mg/kg/day ezetimibe |
| 10 | D12079B | 90 mg/kg/d ILY4008 or ILY4013 |
| 11 | D12079B | 25 mg/kg/d ILY4008 or ILY4013 |
| 12 | D12079B | 90 mg/kg/d ILY4011 or ILY4016 |
| 13 | D12079B | 25 mg/kg/d ILY4011 or ILY4016 |
| 14 | D12079B | 50 mg/kg/d Rosiglitazone |

Animals were maintained on the diets for eight weeks. Body weights were recorded weekly. Blood was drawn within 1-2 hrs of lights-on, without fasting. The serum was analyzed for total cholesterol, LDL cholesterol, HDL cholesterol, triglycerides (TG), free fatty acid (FFA) and lysophospholipid (LPC) content.

Statistical analyses were performed using GraphPad Prism 4.03. (GraphPad Software, Inc., San Diego, Calif.). Two sets of statistical analyses were performed. First, the Low Fat Chow, no treatment group was compared by student's two-tailed T-test against the Western Diet, no treatment group. In all figures an “a” above the low fat chow, no treatment column signifies that the values are significantly different (p<0.05) from the Western diet, no treatment group. Second, all treatment groups on the Western diet were compared to the no-treatment group on that diet by 1-way ANOVA, followed by a Dunnett's post-test. A “b” above a graph column signifies that the values are significantly different (p<0.05) versus the no-treatment group on that diet.

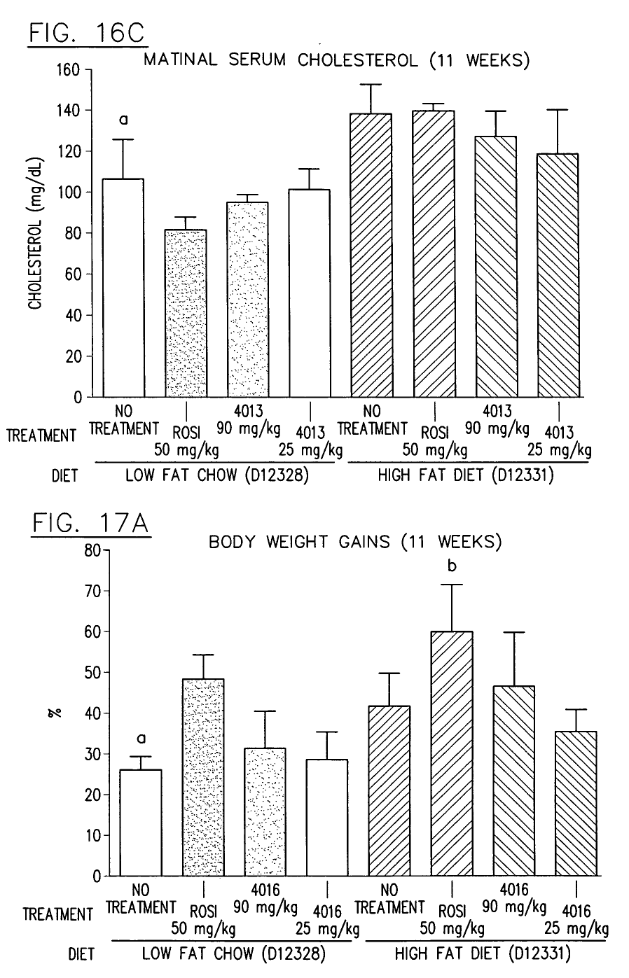

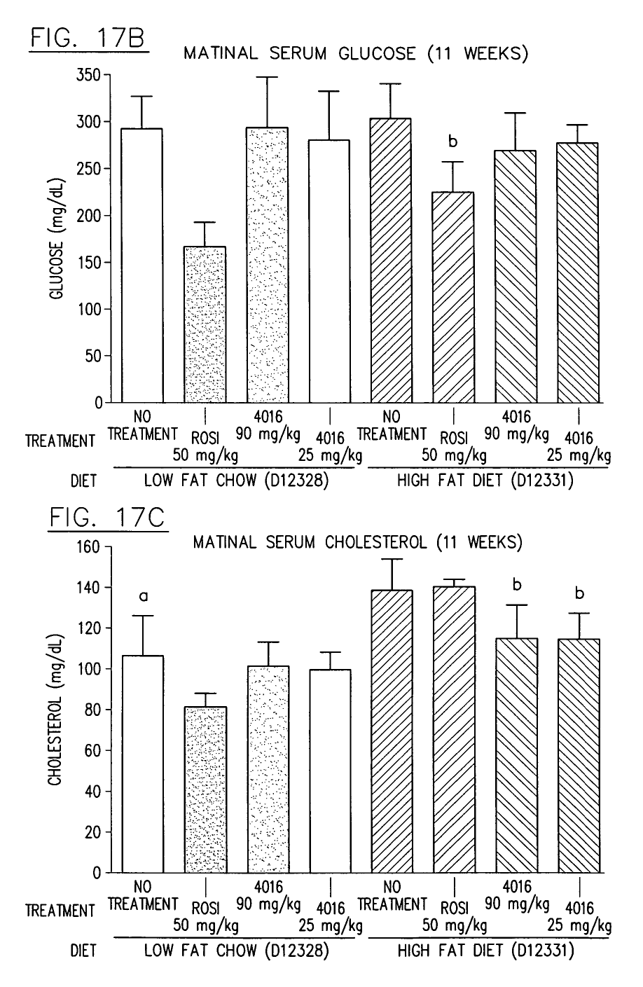

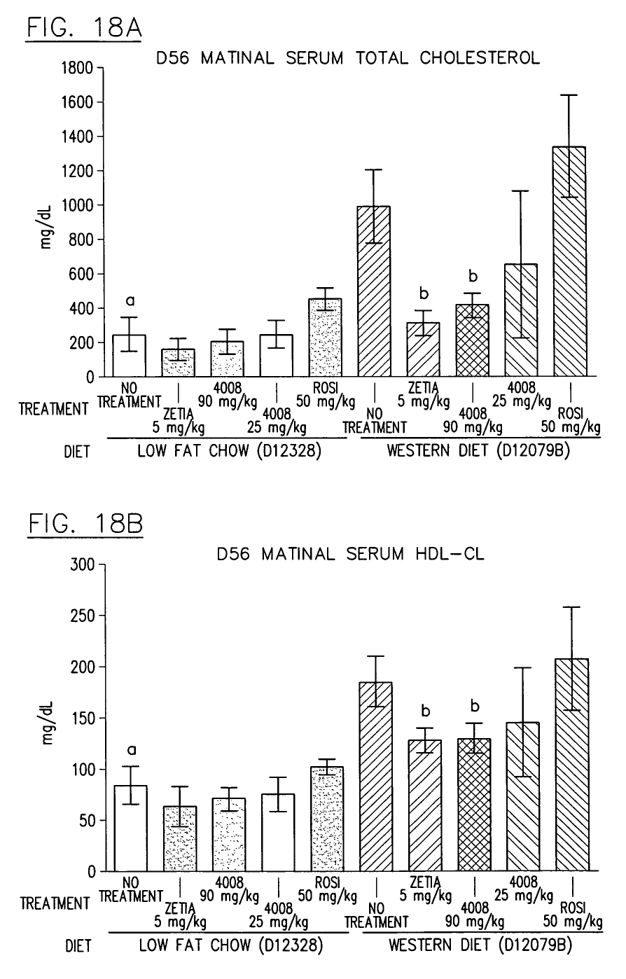

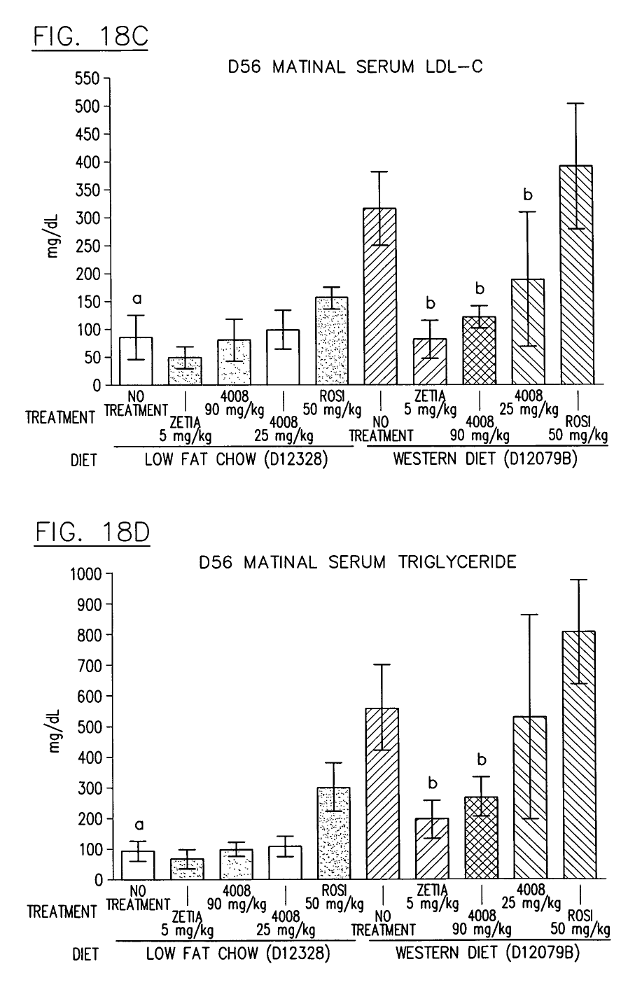

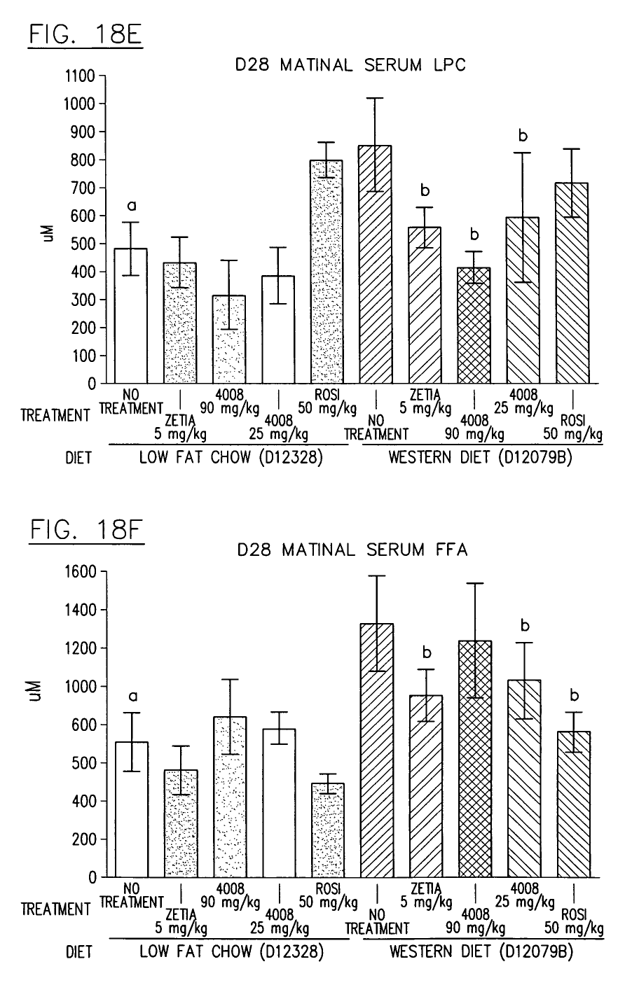

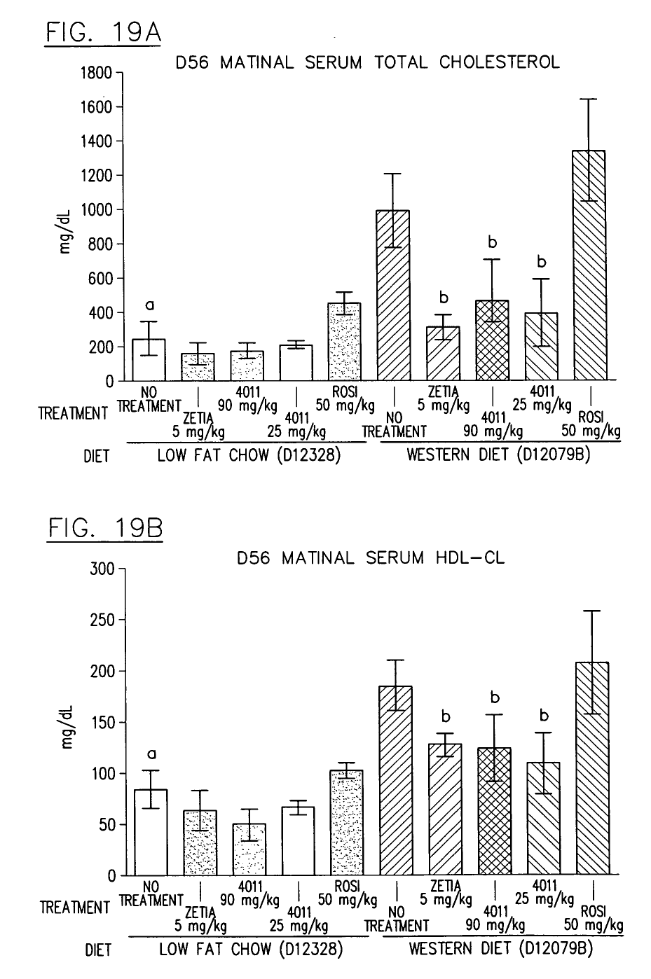

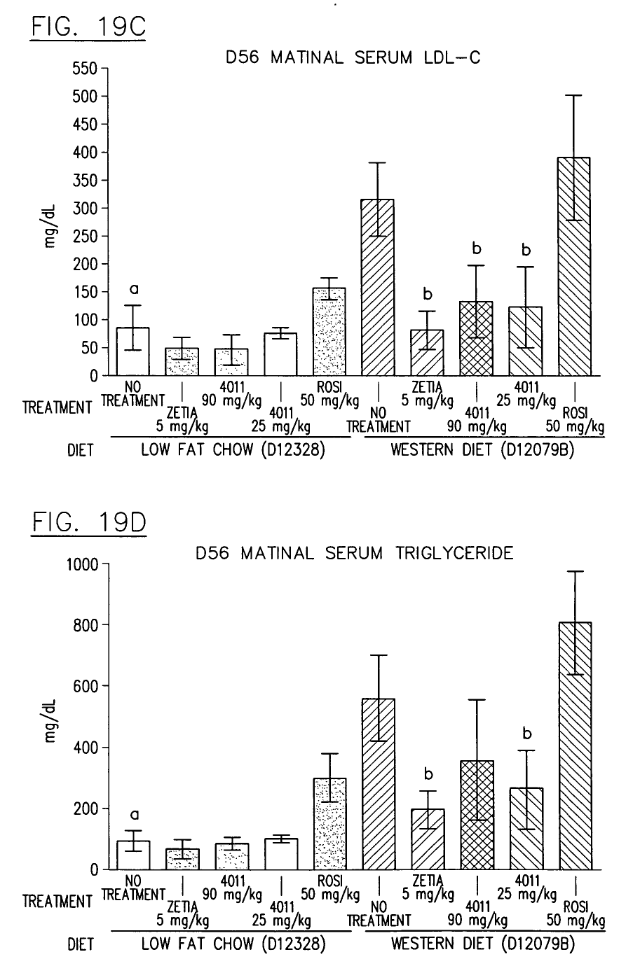

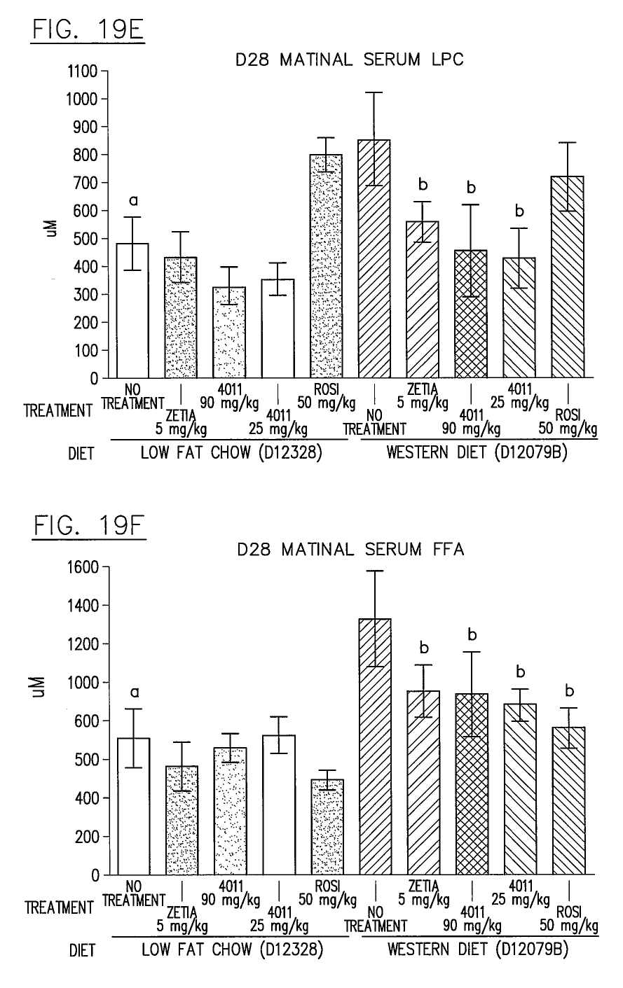

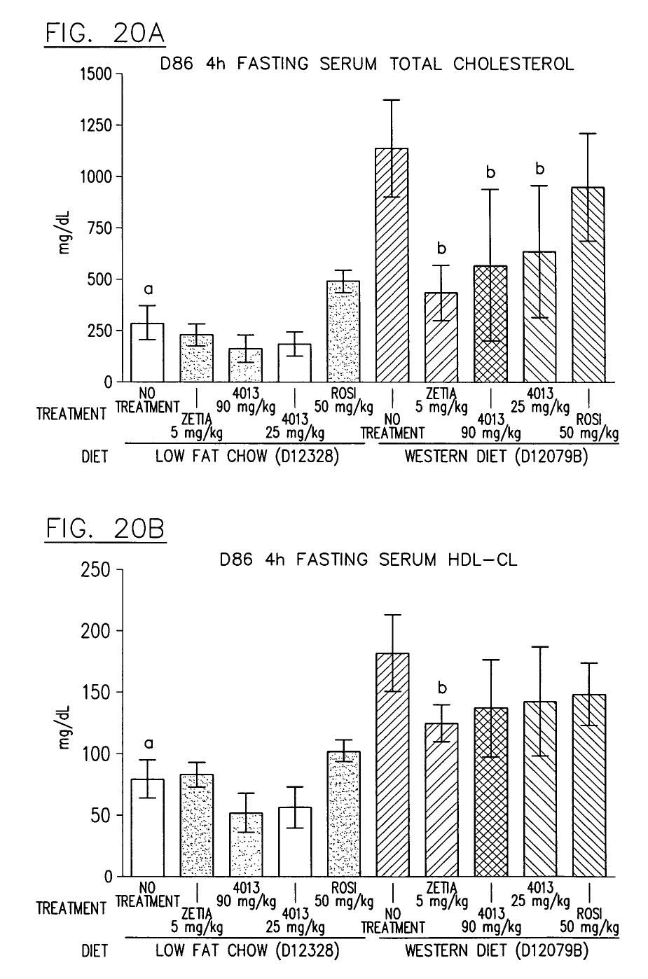

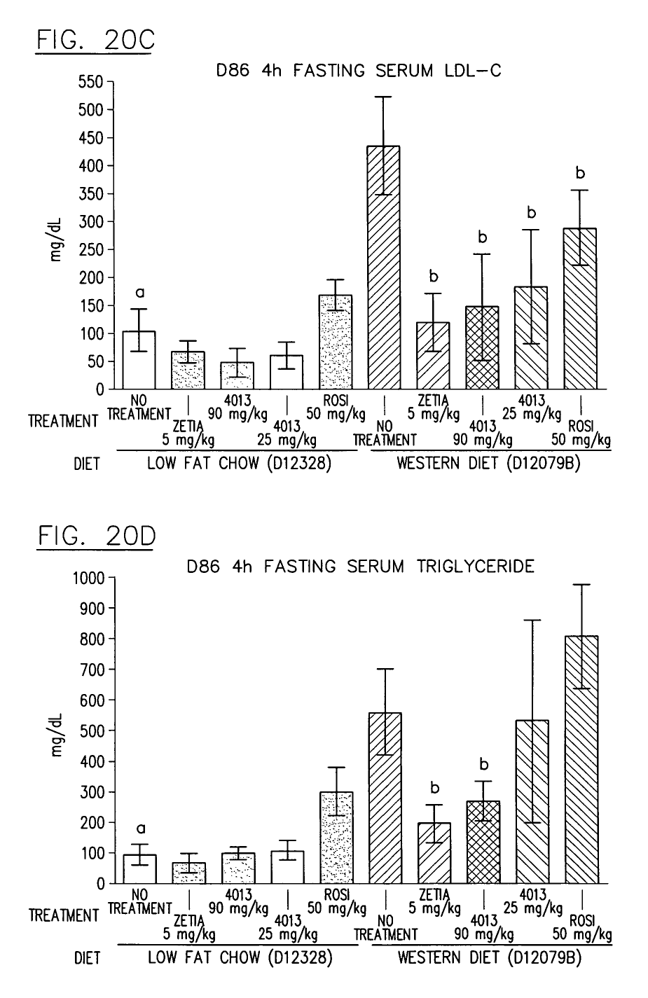

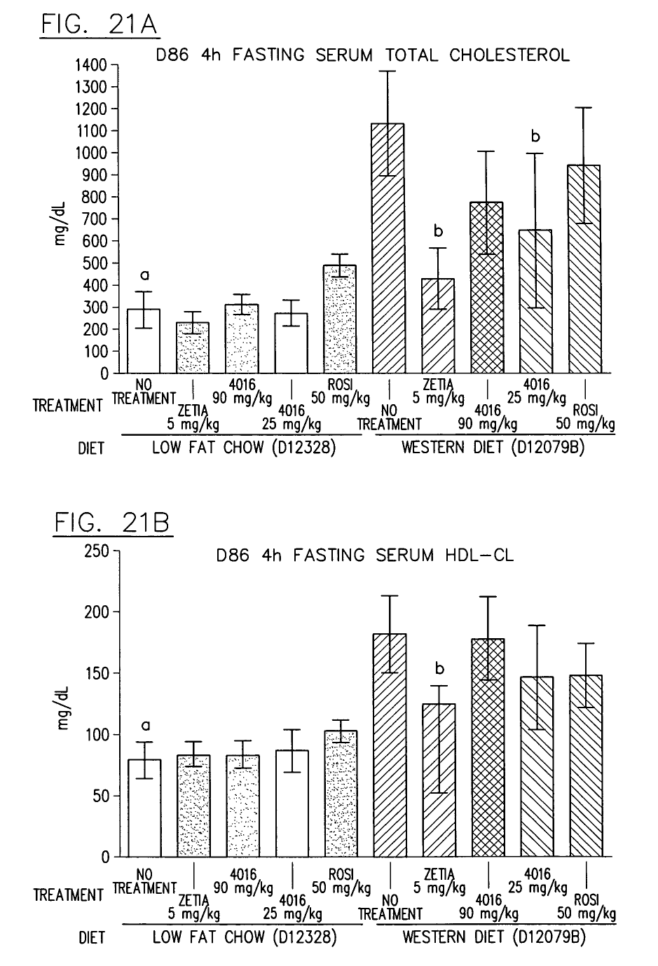

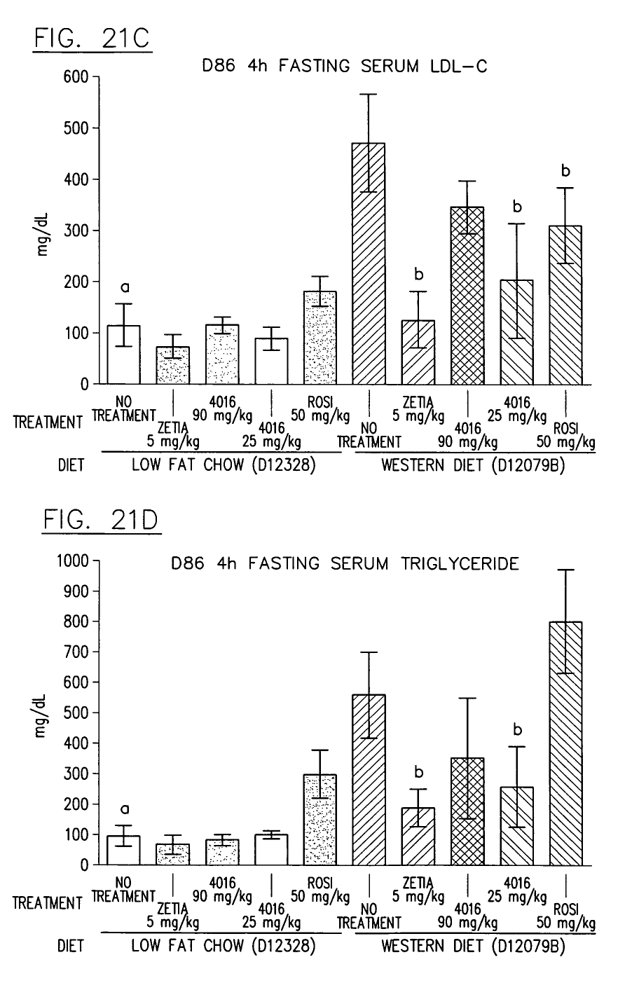

Results for Test Article ILY4008 (ILY-V-26) are shown in FIGS. 18A, 18B, 18C, 18D, 18E and 18F. Results for Test Article ILY4011 (ILY-V-30) are shown in FIGS. 19A, 19B, 19C, 19D, 19E and 19F. Results for Test Article ILY4013 (ILY-V-32) are shown in FIGS. 20A, 20B, 20C and 20D. Results for Test Article ILY4016 (ILY-IV-40) are shown in FIGS. 21A, 21B, 21C and 21D.

No or little effect was observed when animals fed a low fat control diet were compared to animals fed a low fat control diet containing ILY4008, ILY4011, ILY4013 or ILY4016. This observation suggests that some embodiments provide efficacy under high-risk diet conditions yet have no observable effect under lower risk diet conditions.

### Example 21

### NONcNZO10/LTJ MOUSE MODEL OF TYPE II DIABETES

The NONcNZO10/LtJ mouse strain (Jackson Labs, Bar Harbor Me.) is a recombinant congenic strain developed specifically to model human Type 2 diabetes. Although other mouse strains with specific defects in the leptin signaling pathway (for example BKS.Cg-m+/+Leprdb/J, B6.V-Lepob/J and KK.Cg-Ay/J are excellent models of monogenic obesity and useful for researching type 2 diabetes, they do not reflect the more common human obesity-induced diabetes (diabesity) syndromes. Common human diabesity syndromes are polygenic, not monogenic, and the clinical phenotypes of the monogenic models are extreme: massive obesity and hyperphagia, either extremely high or no leptin in circulation, and extreme hyperinsulinism. In contrast, NONcNZO10/LtJ has moderate behavioral and endocrine phenotypes, and males exhibit a maturity-onset transition from impaired glucose tolerance to a stable non-fasting hyperglycemia without hyperphagia or reproductive failure, and only moderately elevated insulin and leptin concentrations in plasma (Leiter, E H, et al. (2005) “Differential Endocrine Responses to Rosiglitazone Therapy in New Mouse Models of Type 2 Diabetes”, Endocrinology, Leiter, E H and Reifsnyder, P C (2004) “Differential levels of diabetogenic stress in two new mouse models of obesity and type 2 diabetes”, Diabetes 53 Suppl 1: S4-11). Also in contrast to the diet-induced obesity (DIO) model used in other studies, NONcNZO10/LtJ male mice show robust hyperglycemia and elevated insulin when fed diets that have only moderately increased amount of fat compared to standard laboratory rodent chow. This model was used to investigate the effects of indole and indole-related Test Articles. Avandia (rosiglitazone) was used as a control test article.

Male NONcNZO10/LtJ mice, five weeks of age, were obtained from Jackson Labs (Bar Harbor, Me.). Upon arrival, the animals were placed on Laboratory Rodent Diet 5K20 (Purina Mills, Inc., St. Louis, Mo.). Diet and water was provided ad libitum throughout the course of the study. Animals were acclimated for at least four weeks, and then weighed on study day (1). Animals with outlying weights were removed from the study. The remaining animals were randomized by weight into six groups of seven animals each. Each group of animals was placed on diets with and without test articles as described in Table 13. All diets were provided by Research Diets (New Brunswick, N.J.). Maltodextrin (5% by weight) was added at Research Diets to each diet to aid reformulation into pellets after the addition of test articles into the 5K20 diet.

The test article content was calculated such that ad libitum consumption by the animals would deliver doses (in mgs Test Article per Kg animal weight per day) approximating those listed in Table 13.

Animals were maintained on the diets for up to two months. Body weights were recorded weekly. Blood was drawn by retroorbital bleeding. For these blood draws, the animals were fasted overnight. The serum was analyzed for glucose, insulin, leptin, total cholesterol and triglyceride (TG) content.

TABLE 13
NONcNZO10/LtJ Mouse Model of Type II Diabetes Assay Diets

| Group | Diet | Added Test Article |
|---|---|---|
| 1 | 5K20 | No added Test Article |
| 2 | 5K20 | 50 mg/kg/d Rosiglitazone |
| 3 | 5K20 | 90 mg/kg/d ILY4008 or ILY4013 |
| 4 | 5K20 | 25 mg/kg/d ILY4008 or ILY4013 |
| 5 | 5K20 | 90 mg/kg/d ILY4011 or ILY4016 |
| 6 | 5K20 | 25 mg/kg/d ILY4011 or ILY4016 |

Statistical analyses were performed using GraphPad Prism 4.03. (GraphPad Software, Inc., San Diego, Calif.). In all figures an “a” above a graph column signifies that the values are significantly different (p<0.05) by 1-way ANOVA, followed by a Dunnett's post-test versus the group fed 5K20 with no test article added.

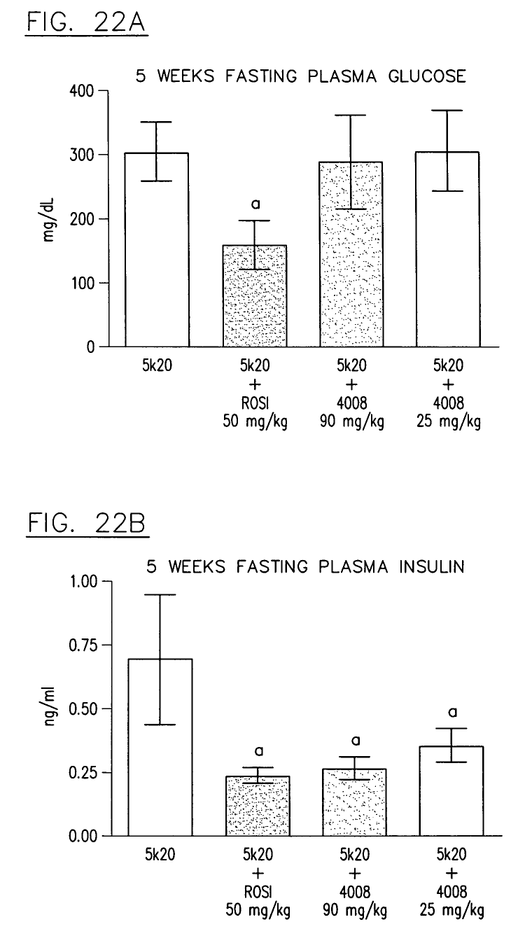

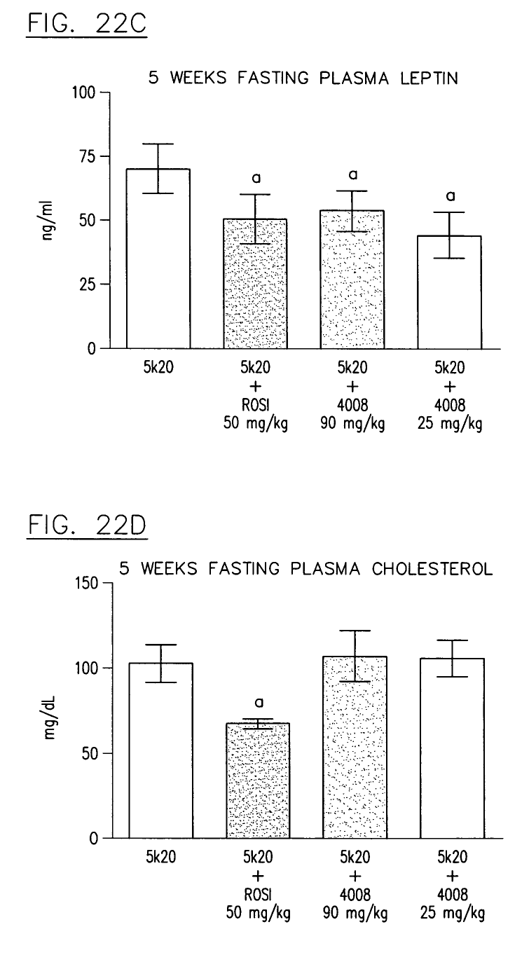

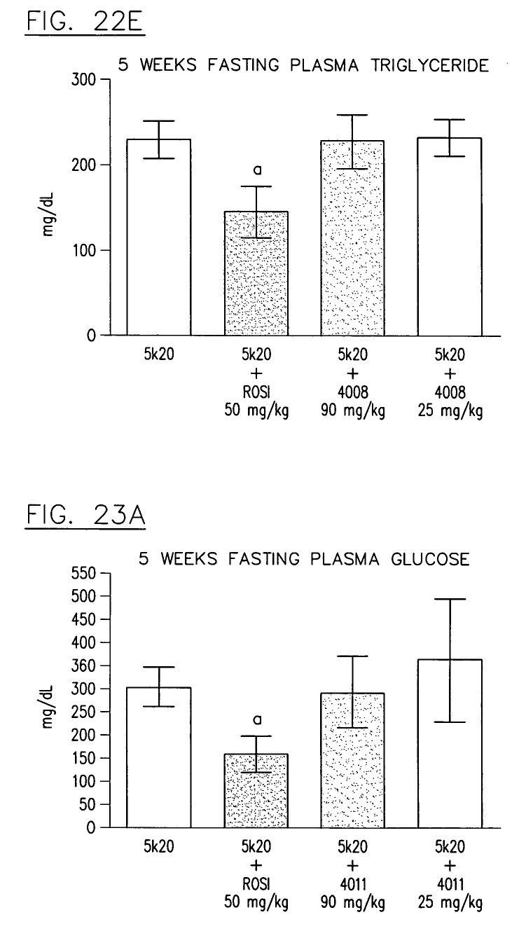

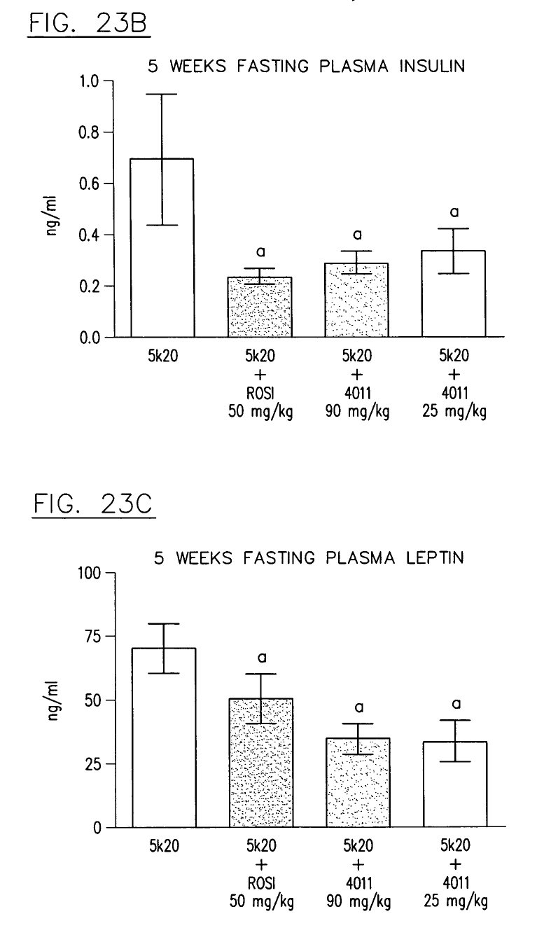

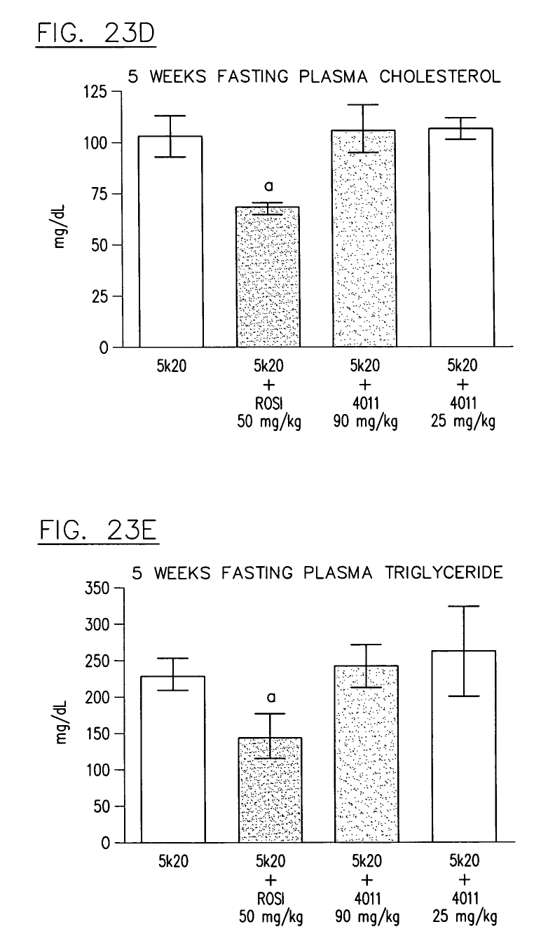

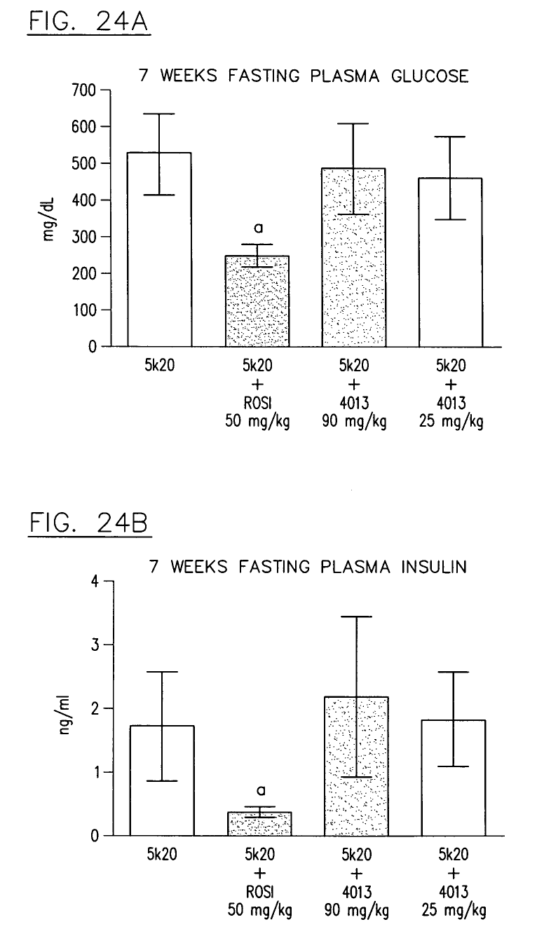

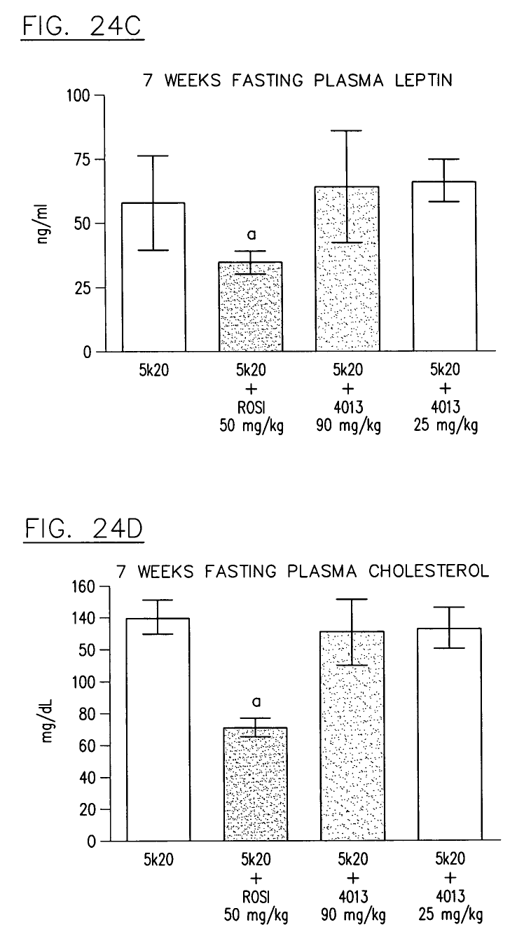

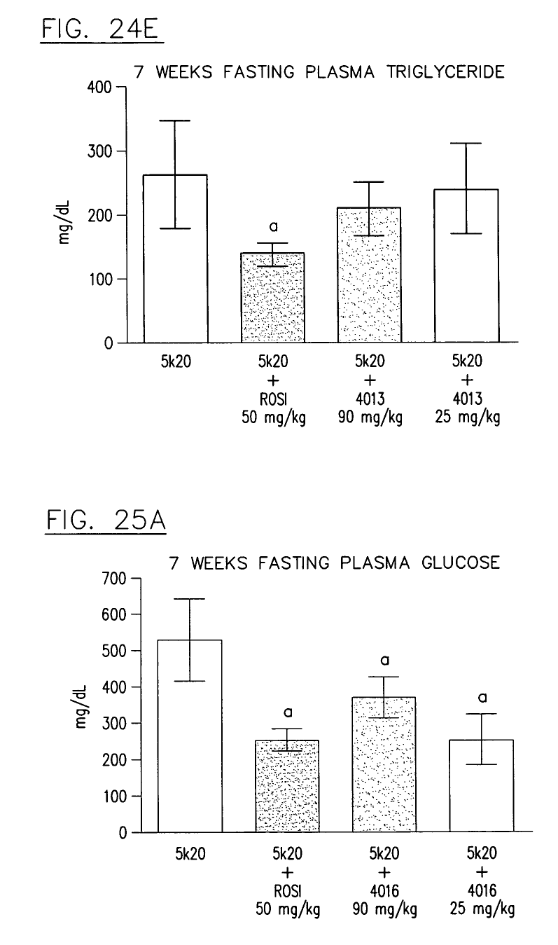

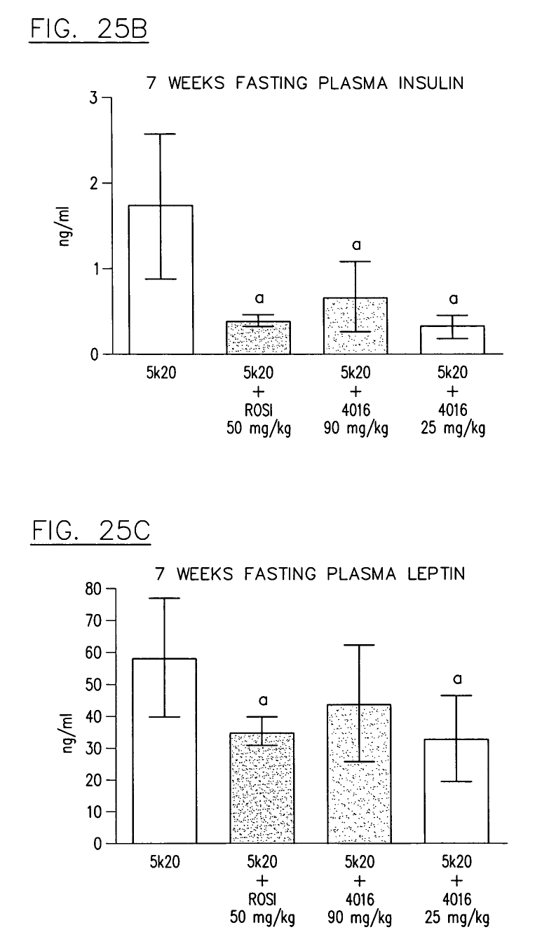

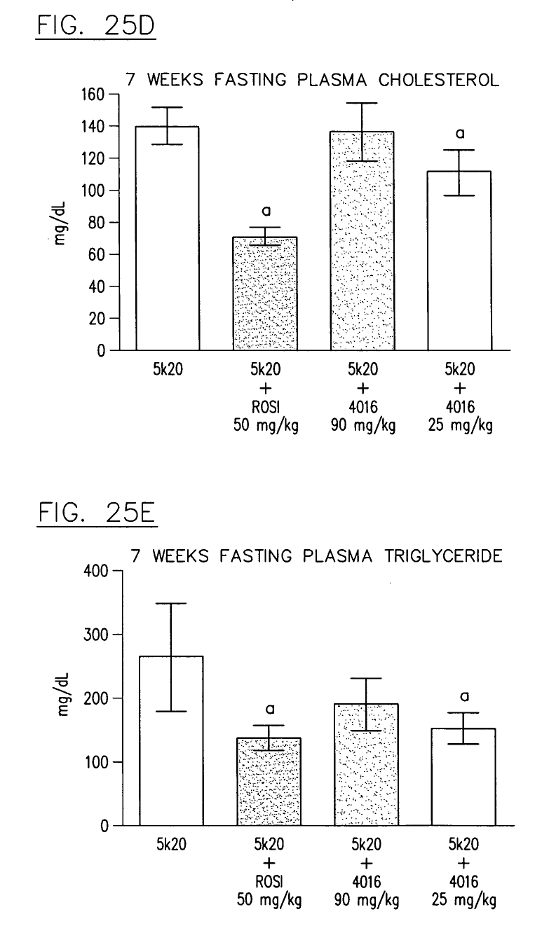

Results for Test Article ILY4008 (ILY-V-26) are shown in FIGS. 22A, 22B, 22C, 22D and 22E. Results for Test Article ILY4011 (ILY-V-30) are shown in FIGS. 23A, 23B, 23C, 23D and 23E. Results for Test Article ILY4013 (ILY-V-32) are shown in FIGS. 24A, 24B, 24C, 24D and 24E. Results for Test Article ILY4016 (ILY-IV-40) are shown in FIGS. 25A, 25B, 25C, 25D and 25E.

### Example 22

### HAMSTER DIET-INDUCED DYSLIPIDEMIA

Golden Syrian hamsters become hypercholesterolemic within one week of being fed a standard rodent diet that has been supplemented with 0.5% cholesterol (van Heek, M, et al. (2001) “Ezetimibe selectively inhibits intestinal cholesterol absorption in rodents in the presence and absence of exocrine pancreatic function”, Br J Pharmacol 134: 409-417). In contrast to wild-type mice, hamsters express cholesterol ester transfer protein (CETP) and have a lipid metabolic profile similar to that of humans. Consequently, hamsters are considered to be an excellent non-primate model of human lipid and cholesterol metabolism (Spady, D K and Dietschy, J M (1988) “Interaction of dietary cholesterol and triglycerides in the regulation of hepatic low density lipoprotein transport in the hamster”, J Clin Invest 81: 300-309, Spady, D K and Dietschy, J M (1989) “Interaction of aging and dietary fat in the regulation of low density lipoprotein transport in the hamster”, J Lipid Res 30: 559-569). This model was used to investigate the effects of indole and indole-related Test Articles. Zetia (ezetimibe) was used as a control test article. The Test Article content was calculated such that ad libitum consumption by the animals would deliver doses (in mg of Test Article per kg animal weight per day) approximating those listed in Table 14.

Golden Syrian hamsters were placed on Laboratory Rodent Diet 5001 (Purina Mills, Inc., St. Louis, Mo.) for a ten-day acclimation period. Diet and water was provided ad libitum throughout the course of the study. After acclimation, blood was drawn and serum cholesterol levels were measured. Animals with outlying cholesterol levels were removed from the study and the remaining animals were randomized by matinal serum cholesterol into eight groups of six animals each. Each group of animals was placed on diets with and without test articles as described in Table 14. All diets were provided by Research Diets (New Brunswick, N.J.). Blood draws via retro-orbital bleeding on lightly sedated hamsters were performed within two hours of lights on at baseline (pre-diet dosing, for randomization), and on study days 7, 14, and 21. The final blood draw, on day 28, was performed through terminal cardiocentesis after 24 hr food fasting. Results from the day 28 blood draw were thus not included in the 2-way ANOVA analysis. The serum was analyzed for total cholesterol, LDL-cholesterol, HDL-cholesterol and triglyceride (TG) content.

TABLE 14
Hamster Diet-Induced Dyslipidemia Assay Diets

| | | | | Dose |
|---|---|---|---|---|
| | Test | | | (mg/kg |
| Group | Article | Base Diet | Dose (mg/kg) | of diet) |
| 1 | none | Purina 5001 | ad lib. | N/A |
| 2 | none | Purina 5001 + | ad lib | N/A |
| | | 0.5% Cholesterol | | |
| 3 | ezetimibe | Purina 5001 + | ad lib (estimated 1 | 10 |
| | | 0.5% Cholesterol | mg ezetimibe/kg/d). | |
| 4 | ILY4008 | Purina 5001 + | ad lib (estimated 90 | 900 |
| | | 0.5% Cholesterol | mg ezetimibe/kg/d). | |
| 5 | ILY4011 | Purina 5001 + | ad lib (estimated 90 | 900 |
| | | 0.5% Cholesterol | mg ezetimibe/kg/d). | |
| 6 | ILY4013 | Purina 5001 + | ad lib (estimated 90 | 900 |
| | | 0.5% Cholesterol | mg ezetimibe/kg/d). | |
| 7 | ILY4016 | Purina 5001 + | ad lib (estimated 90 | 900 |
| | | 0.5% Cholesterol | mg ezetimibe/kg/d). | |
| 8 | ILY4017 | Purina 5001 + | ad lib (estimated 90 | 900 |
| | | 0.5% Cholesterol | mg ezetimibe/kg/d). | |

Statistical analyses were performed using GraphPad Prism 4.03. (GraphPad Software, Inc., San Diego, Calif.). In all figures “*” above a graph column signifies that the values are significantly different (p<0.05) versus group 2 (Purina 5001 supplemented with 0.5% cholesterol and no test article added) by 2-way ANOVA, followed by a Bonferroni's post-test. Day 28 (fasting) values were not included in the 2-way ANOVA analysis.

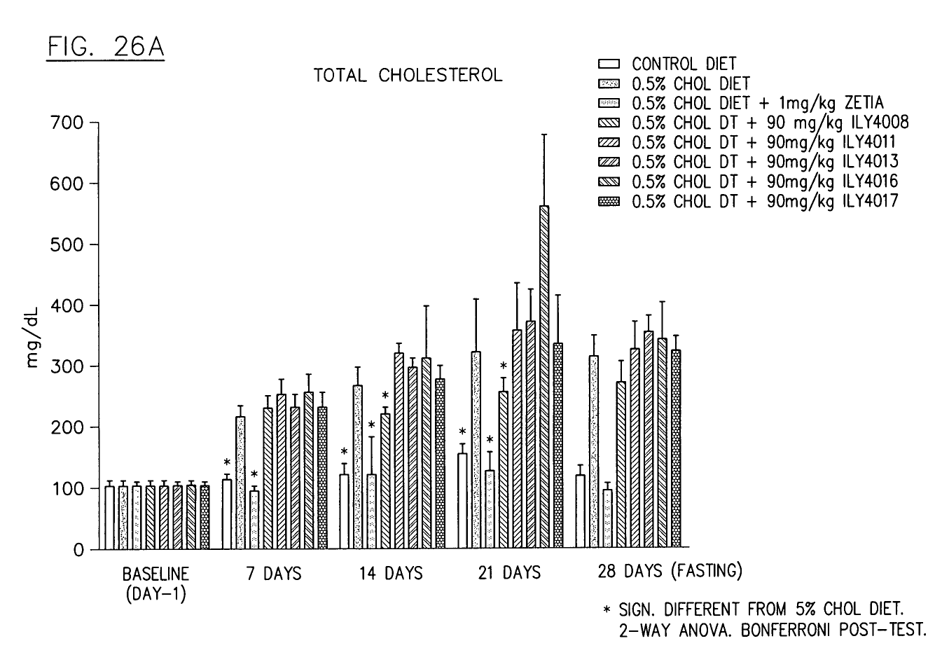

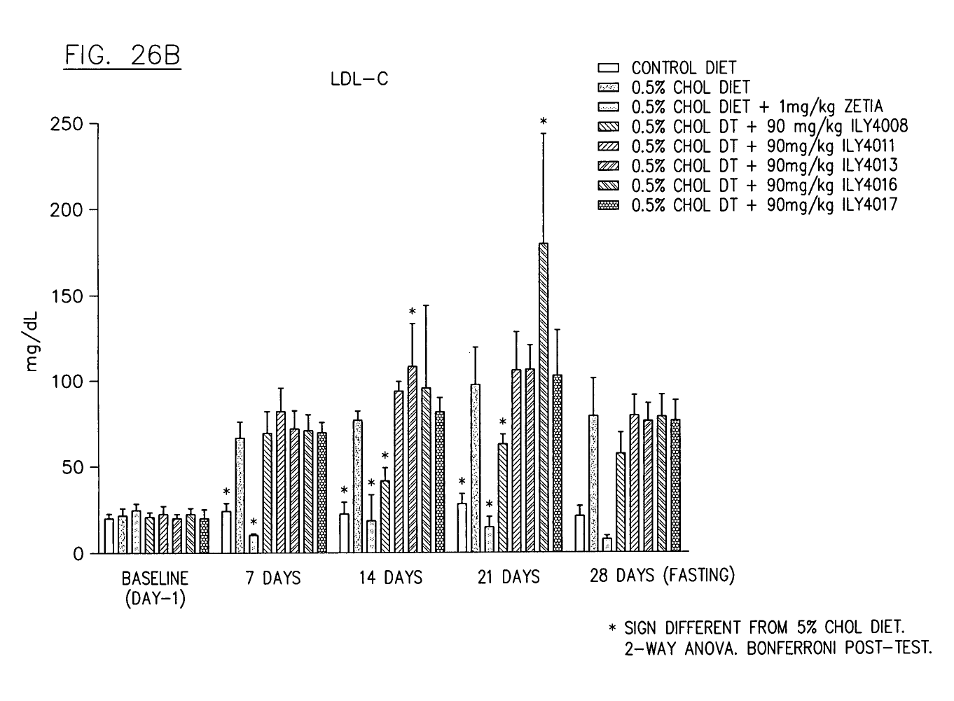

The results for Test Articles ILY4016 (ILY-IV-40), Test Article ILY4008 (ILY-V-26), Test Article ILY4013 (ILY-V-32), Test Article ILY4011 (ILY-V-30), and Test Article ILY4017 (ILY-V-37) are shown in FIGS. 26A and 26B.

### Example 23

### TOXICOLOGY

The purpose of this study was to evaluate the toxicity of indole and indole-related Test Articles when administered daily via oral gavage to mice for 5 consecutive days.

Assessment of toxicity was based on mortality; clinical signs, body weight, food consumption, clinical pathology, and macroscopic pathology data.

All animals survived to scheduled sacrifice. There were no treatment-related clinical observations. There were no remarkable changes in the body weight or food consumption data.

The clinical pathology data were generally unremarkable and similar among the groups. There were no differences between the vehicle control group and the treated groups that could be attributed to the administration of any of the test articles (ILY4008, ILY4011, ILY4013, ILY4016, and ILY4017).

There were no macroscopic findings at necropsy. There was no evidence of toxicity associated with any of the test articles at the dose levels use in this study.

The observation of no toxicity is consistent with embodiments having a characteristic property of low absorbtion or non-absorbtion.

All publications, patents, and patent applications mentioned in this specification are herein incorporated by reference to the same extent as if each individual publication, patent, or patent application was specifically and individually indicated to be incorporated by reference.

It can be appreciated to one of ordinary skill in the art that many changes and modifications can be made thereto without departing from the spirit or scope of the appended claims, and such changes and modifications are contemplated within the scope of the instant invention.

1-68. (canceled)

69. A method of treating a condition comprising:
orally administering to a subject an effective amount of a phospholipase inhibitor, the phospholipase inhibitor inhibiting activity of a secreted, calcium-dependent phospholipase-A₂ present in a gastrointestinal lumen, the phospholipase inhibitor comprising a substituted organic compound, or a salt thereof, the substituted organic compound comprising two or more independently selected phospholipase inhibiting moieties, Z, linked by independently selected linking moieties, L, to a multifunctional bridge moiety, as represented by formula (D-I) 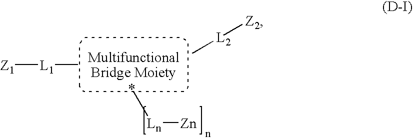
with n being an integer ranging from 0 to 10, and the multifunctional bridge moiety having at least (n+2) reactive sites to which the two or more phospholipase inhibiting moieties are bonded, and
localizing the inhibitor in a gastrointestinal lumen such that following administration, the phospholipase inhibitor remains in and passes through the gastrointestinal lumen.

70. The method of claim 69 wherein the multifunctional bridge moiety is other than an oligomer moiety or a polymer moiety.

71. The method of claim 69 wherein the multifunctional bridge moiety is selected from the group consisting of alkyl, phenyl, aryl, alkenyl, alkynyl, heterocyclic, amine, ether, sulfide, disulfide, hydrazine, and any of the foregoing substituted with oxygen, sulfur, sulfonyl, phosphonyl, hydroxyl, alkoxyl, amine, thiol, ether, carbonyl, carboxyl, ester, amide, alkyl, alkenyl, alkynyl, aryl, heterocyclic, and moieties comprising combinations thereof.

72. The method of claim 69 wherein the phospholipase inhibitor inhibits phospholipase-A₂ IB.

73. The method of claim 69 wherein essentially all of the phospholipase inhibitor is localized in the gastrointestinal lumen.

74. The method of claim 69 wherein the phospholipase inhibitor is localized in the gastrointestinal lumen such that upon administration to the subject, at least about 80% of the phospholipase inhibitor remains in the gastrointestinal lumen.

75. The method of claim 69 wherein the phospholipase inhibitor is localized in the gastrointestinal lumen such that upon administration to the subject, at least about 90% of the phospholipase inhibitor remains in the gastrointestinal lumen.

76. The method of claim 69 wherein the phospholipase inhibitor is stable while passing through at least the stomach, the duodenum and the small intestine of the gastrointestinal tract.

77. The method of claim 69 wherein the phospholipase inhibitor inhibits activity of a phosholipase-A₂, but essentially does not inhibit other gastrointestinal mucosal membrane-bound phospholipases.

78. The method of claim 69 wherein the condition is selected from the group consisting of an insulin-related condition, a weight-related condition, a cholesterol-related condition, and a dyslipidemia-related condition.

79. The method of claim 78 wherein an effective amount of the phospholipase inhibitor is used to inhibit at least about 30% of phospholipase-A₂ activity.

80. The method of claim 78 wherein an effective amount of the phospholipase inhibitor is used to inhibit at least about 50% of phospholipase-A₂ activity.

81. The method of claim 78 wherein an effective amount of the phospholipase inhibitor is used to inhibit at least about 70% of phospholipase-A₂ activity.

82. The method of claim 78 wherein the subject is a human.

83. The method of claim 69 wherein n is an integer ranging from 0 to 4.

84. The method of claim 69 wherein the phospholipase inhibitor comprises a dimerized indole moiety.

85. The method of claim 69 wherein the phospholipase inhibitor comprises a trimerized indole moiety.

86. A pharmaceutical composition effective to treat a condition in a subject, the pharmaceutical composition comprising a phospholipase inhibitor,
the phospholipase inhibitor comprising a substituted organic compound, or a salt thereof, the substituted organic compound comprising two or more independently selected phospholipase inhibiting moieties, Z, linked by independently selected linking moieties, L, to a multifunctional bridge moiety, as represented by formula (D-I) 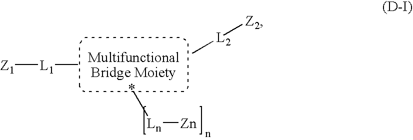
with n being an integer ranging from 0 to 10 and the multifunctional bridge moiety having at least (n+2) reactive sites to which the two or more phospholipase inhibiting moieties are bonded,
the phospholipase inhibitor inhibiting activity of a secreted, calcium-dependent phospholipase-A₂ present in a gastrointestinal lumen, and
the phospholipase inhibitor being localized in the gastrointestinal lumen such that following administration to the subject, the phospholipase inhibitor remains in and passes through the gastrointestinal lumen.

87. The composition of claim 86 wherein the multifunctional bridge moiety is other than an oligomer moiety or a polymer moiety.

88. The composition of claim 86 wherein the multifunctional bridge moiety is selected from the group consisting of alkyl, phenyl, aryl, alkenyl, alkynyl, heterocyclic, amine, ether, sulfide, disulfide, hydrazine, and any of the foregoing substituted with oxygen, sulfur, sulfonyl, phosphonyl, hydroxyl, alkoxyl, amine, thiol, ether, carbonyl, carboxyl, ester, amide, alkyl, alkenyl, alkynyl, aryl, heterocyclic, and moieties comprising combinations thereof.

89. The composition of claim 86 wherein the phospholipase inhibitor inhibits phospholipase-A₂ IB.

90. The composition of claim 86 wherein the phospholipase inhibitor reversibly inhibits phospholipase-A₂.

91. The composition of claim 86 wherein the phospholipase inhibitor irreversibly inhibits phospholipase-A₂.

92. The composition of claim 86 wherein the phospholipase inhibitor inhibits phospholipase A₂ and phospholipase B.

93. The composition of claim 86 wherein the phospholipase inhibitor essentially does not inhibit a lipase.

94. The composition of claim 86 wherein the phospholipase inhibitor essentially does not inhibit phospholipase B.

95. The composition of claim 86 wherein the phospholipase inhibitor inhibits activity of phospholipase A₂, but essentially does not inhibit other gastrointestinal phospholipases having activity for catabolizing a phospholipid.

96. The composition of claim 86 wherein the phospholipase inhibitor inhibits activity of phospholipase A₂, but essentially does not inhibit other gastrointestinal mucosal membrane-bound phospholipases.

97. The composition of claim 86 wherein the phospholipase inhibitor has a permeability coefficient lower than about −5.

98. The composition of claim 86 wherein the phospholipase inhibitor comprises at least one indole moiety.

99. The composition of claim 98 wherein the condition is selected from the group consisting of an insulin-related condition, a weight-related condition, a cholesterol-related condition, and a dyslipidemia-related condition.

100. The composition of claim 98 wherein n is an integer ranging from 0 to 4.

101. The composition of claim 98 wherein the phospholipase inhibitor comprises a dimerized indole moiety.

102. The composition of claim 98 wherein the phospholipase inhibitor comprises a trimerized indole moiety.

103. The composition of claim 98 wherein the subject is a human.
